(19) United States
(12) Patent Application Publication
(10) Pub. No.: US 2023/0364225 A1
(43) Pub. Date: Nov. 16, 2023

(54) ENHANCED IMMUNOGENIC DNA/RNA COMPOSITIONS AND METHODS

(71) Applicant: MBF Therapeutics, Inc., Ambler, PA (US)

(72) Inventors: Lorraine Keller, Pipersville, PA (US); Malla Padidam, Chalfont, PA (US)

(21) Appl. No.: 18/064,219

(22) Filed: Dec. 9, 2022

Related U.S. Application Data
(63) Continuation-in-part of application No. PCT/US2021/036897, filed on Jun. 11, 2021.
Application No. PCT/US2021/036897 is a continuation-in-part of application No. 16/917,899, filed on Jun. 30, 2020, now Pat. No. 11,130,787.
(60) Provisional application No. 63/038,117, filed on Jun. 11, 2020.

(30) Foreign Application Priority Data
Sep. 30, 2022 (CA) 3179319

(51) Int. Cl. A61K 39/39; A61K 39/215; A61K 39/245; A61K 39/00; A61K 39/145; A61K 39/12

(52) U.S. Cl. CPC A61K 39/39; A61K 39/215; A61K 39/245; A61K 39/0011; A61K 39/145; A61K 39/12; A61K 2039/53; A61K 2039/552; A61K 2039/55505; A61K 2039/55516; A61K 2039/522

(57) ABSTRACT

Provided are pharmaceutical/veterinary compositions including (1) an effective amount of nucleic acid molecule(s) comprising nucleotide sequences encoding (a) disease-causing agent-associated antigen(s) and (b) innate trained immunity immunomodulator(s), and (2) an effective amount of either (a) calcium phosphate nanoparticles associated with the molecules that detectably enhance cellular uptake of the molecules, immunogenicity of the composition, or both, or (b) a nonpathogenic bacterial vector that contains the molecules and detectably promotes delivery of the nucleic acid molecules. Immunomodulators can include signal transducing activator protein sequences, antigen(s) can include immune cell targeting sequences (e.g., alphaherpesvirus glycoprotein D sequences) or internal targeting sequences (such as polyubiquitin sequences), and expression products can include checkpoint inhibitors. Provided also are methods of using compositions to treat or prevent diseases in humans or livestock/companion animals, including viral diseases and cancers.

### CROSS-REFERENCE TO RELATED APPLICATIONS (PRIORITY)

This application claims priority to Canadian Application No. 3,179,319, filed Sep. 30, 2022. This application is a continuation-in-part of PCT/US2021/036897, filed Jun. 11, 2021, which claims priority to U.S. application Ser. No. 16/917,899, filed Jun. 30, 2020 (now issued as U.S. Pat. No. 11,130,787), which claims the benefit of U.S. Provisional Application No. 63/038,117, filed Jun. 11, 2020, each of which being hereby incorporated by reference.

### REFERENCE TO SEQUENCE LISTING SUBMITTED ELECTRONICALLY

This Application references content of an electronically submitted sequence listing submitted electronically as an XML Sequence Listing file with the file name “MBF_GDFUSIONUSTWO_SEQLST_FINAL”; having a file size of 1,243,886 bytes; and a date of creation of Aug. 5, 2023. This Sequence Listing is incorporated herein by reference in its entirety.

### BACKGROUND OF THE INVENTION

Despite over 100 years of research, enhancement of immune responses remains a significant challenge. Delivery of vaccines derived from pathogens is the oldest and still most common immunomodulation method.

Vaccines have been effective against several pathogens. However, many vaccination approaches are not sufficiently protective against re-challenge (i.e., do not lead to sufficient “immunological memory”). Only mall populations of memory immune cells that persist after initial infection or vaccination is a challenge. E.g., Panagioti et al. Front. Immunol., 16 Feb. 2018. Also, most vaccination methods rely entirely on the production of antibodies from B cells (a humoral immune response). However, many viruses enter host cells within minutes, becoming sequestered from contact with B cells/antibodies. (See Hernáez B, Guerra M, Salas M L, Andrés G. PLoS Pathog. 2016; 12 (4)) and Dou D et al. Front Immunol. 2018; 9:1581).

Possibly because of these and similar shortcomings, many diseases have been extensively studied for vaccine development but without established clinical success to date (e.g., dengue, Helicobacter pylori, and malaria). Further, several on-market vaccines have limited efficacy, especially in certain populations, against pathogens, such as influenza and varicella (see, e.g., Kim et al., Clin Exp Immunol. 2017 January; 187(1): 71-81). Immunologists often characterize vaccines that are only partially effective against subsequent challenges as “leaky vaccines.” (See, e.g., Edlefsen P T. Comput Math Methods Med. 2014). The “leaky” characterization reflects that while such vaccines can elicit an immune response, the response is neither sustaining or complete (sterilizing), and, thus, is not sufficient to block spread of the disease.

Breakthroughs in understanding “cellular” (T cell-mediated) immunity, innate immunity, and modulation of immune checkpoint systems, have provided new tools for improvement of vaccines. See, e.g., Lasaro et al., Human Vaccines, 5:1, 6-14 (2009). E.g., in 2010, US FDA approved the first dendritic cell (DC)-based immunotherapy (Provenge®) for advanced prostate cancers. However, only a few DC-based treatment strategies have since been tested for viral conditions in clinical trials and efficacy in trials targeting DCs has been, “at best, mixed, and often lacking, particularly with respect to reductions in viral load” as “immunological responses[in such trials] were either weak or transient and, more importantly, reduction in viral load has been observed in only a few of these studies.” Atanley E et al. Expert Rev Clin Immunol. 2014; 10(6):801-813. Other research shows DCs are often ineffective at antigen presentation in contexts (e.g., where immune system components are deficient). See Chen P, et al. Hum Vaccin Immunother. 2016; 12(3):612-622. There also have been significant recent breakthroughs in understanding that DCs, NKCs, and other immune cells, previously characterized as innate immune cells, exhibit or support immunological functions similar to adaptive immunity cells (e.g., B cells and T cells), and play a special and often critical role in generating effective immune responses. Such cells are often referred to as “innate trained immunity cells.” See, e.g., Eljaszewicz et al. J Allergy Clin Immunol. 2021 May; 147(5):1865-1877; Mantovani et al., 2020, N Engl J Med 2020; 383:1078-1080; Mincham et al. Front Immunol. 2020 Dec. 2; 11: 601494; Ldrias et al. Front Microbiol. 2020 Jan. 10; 10:2924; and Eljaszewicz et al. J Allergy Clin Immunol. 2021 May; 147(5):1865-1877.

Research also now indicates humoral responses may not be sufficient, e.g., finding T cell immunity is important to effectively treating several conditions. See, e.g., Pangioti et al, supra. However, the number of effector cells required for an effective T cell response are “exponentially larger” than the number of cells required for an effective B cell response. See, e.g., Pennock et al., Trends in Immunology. Vol. 37, ISS. 3, p170-180, March 2016. As such, while traditional vaccines can produce the required number of effectors for effective B cell immunity, they are not effective for T cell immunity. Id. However, understanding of T cell immunology is still unfolding. E.g., until recently, it was believed that induction of CD8+ cytotoxic T lymphocytes was sufficient to provide protective memory, but recent work indicates CD4+ memory T cells are important if not essential (see Zander et al. Immunity. 2019; 51(6):1028-1042.e4 & Ahrends et al. Nat Commun 10, 5531 (2019)).

An area of significant research is the field of recombinant “nucleic acid vaccines,” which are typically viruses or plasmids containing DNA/RNA sequences encoding antigen(s). Despite a huge number of patent disclosures, and several clinical trials, no DNA vaccines have been approved for human use, and only 2 have been approved for veterinary use (See, Hobernik and Bros, Int J Mol Sci. 2018 November; 19(11): 3605). Hobernik and Bros indicate that the impeded development of DNA vaccines may be attributable to the fact that while nucleic acid vaccines known to date have been able to evoke detectable levels of cellular and humoral responses, such responses have not been sufficient to elicit actual clinical benefits. Manickan et al, Crit Rev Immunol. 2017; 37 (2-6):483-498, reviews use of DNA vaccines since 1993, and similarly questions the efficacy of DNA vaccines (“It seems doubtful if DNA vaccines will replace currently effective vaccines . . . ”).

Efforts to address such failures have led some to develop several “DNA vaccine optimization strategies.” Approaches include optimization of promoters, optimization of antigens (e.g., codon optimization), inclusion of amino acid or nucleotide sequences that stimulate the immune system (e.g., A/T-rich sequences, CpG oligonucleotide sequences); and co-administration with various adjuvants (e.g., cytokines) or enhancers (e.g., transcription factors). Li and Petrovsky, review similar strategies including novel plasmid vectors, codon optimization to enhance antigen expression, new gene transfection systems or electroporation to increase delivery efficiency, use of protein or live virus vector boosting regimens to maximize immune stimulation, and formulation methods. Expert Rev Vaccines. 2016; 15(3):313-29. Other specific examples of such improvements include use of hybrid viral/eukaryotic promoters; use of expression systems optimized for antigen-presenting cell (“APC”) expression; use of codon optimization; use of linkers to separate antigens; use of nuclear localization signals to facilitate nuclear entry of the DNA (See, e.g., Dean et al., Gene Ther. 2005 June; 12(11): 881-890, and Bai et al., Biosci Rep. 2017 Dec. 22; 37 (6): BSR20160616); fusion protein constructs with a MHC Class II invariant chain sequence; co-administration of expression vectors encoding factors that enhance APC activation and/or T cell attraction/polarization; inclusion of intrinsic inhibitory elements (e.g., insertion of A/T-rich sequences), and inclusion of additional adjuvant (e.g., cytokine); or secondary sequences encoding such factors, which often are designed as a polycistronic construct by incorporation of polycistronic-enabling factors, such as an IRES (internal ribosome entry site) sequence and/or 2A self-cleaving peptide sequence (e.g., P2A, T2A, E2A, or F2A) (e.g., Pelletier et al, Nature. 1988 Jul. 28; 334(6180):320-5, Jang et al. J Virol. 1988 August; 62(8):2636-43, Ibrahimi et al., Hum Gene Ther. 2009 August; 20(8):845-60; and Kim et al., PLoS One. 2011; 6 (4): e18556; Epub 2011 Apr. 29). Understanding that antigens are processed in the proteasome of APCs, another proposed approach is the use of proteasome-targeting sequences (e.g., ubiquitin “tags”) to direct antigen/tag “fusion” proteins/peptides expressed from nucleic acid vaccines (e.g., US Patent Publication No. US20020058021). Nonetheless, no single approach appears to have overcome the shortcomings associated with 1^{st}generation DNA vaccines.

Understanding checkpoint regulation of the immune system also has led to the development of new immunotherapies (e.g., modulators of CTLA4, PD-1, and PD-L1), particularly in cancer treatment (see, e.g., Naidoo et al., Hematol Oncol Clin North Am. 2014 June; 28(3):585-600, Haanen and Robert, Prog Tumor Res. 2015; 42:55-66; Dine et al., Asia Pac J Oncol Nurs. 2017 April-June; 4(2): 127-135, and Kourie et al., Immunotherapy. 2017 June; 9(8):647-657). However, while checkpoint blockade may be universally effective against a broad spectrum of cancer types and is mostly unrestricted by the mutation status of certain genes, only a minority of patients achieve a complete response. E.g., Li et al., Curr Med Chem. 2019; 26(17):3009-3025. All checkpoint inhibitors used in human therapy to date are monoclonal antibodies. Also, most major checkpoint inhibitors are associated with relatively high levels of adverse events. El Osta et al., Crit Rev Oncol Hematol. 2017 November; 119:1-12.

Several patent publications describe/propose combinations of checkpoint inhibitors, antigens, and immunostimulators to improve immune responses. E.g., WO2017177907, describes recombinant proteins comprising an immune checkpoint molecule segment, an auxiliary T cell epitope segment, and an immunostimulatory molecule segment. U.S. Pat. No. 9,474,717 describes DNA constructs, delivered with calcium phosphate nanoparticles, comprising an immunomodulatory element, such as a checkpoint inhibitor (e.g., PDL1), sometimes combined with targeting or immunostimulatory elements. WO2018140890 similarly discloses nucleic acid constructs encoding a checkpoint inhibitor (e.g., PD-L1, PD-L2), targeting factors, immunomodulatory elements (e.g., cytokines), and antigens. WO2019071032 discloses nucleic acid constructs comprising checkpoint inhibitor sequences, immunomodulatory sequences, targeting factors, and multiple antigen-encoding sequences.

Herpes glycoprotein D (“gD”) is a well-characterized envelope glycoprotein and receptor-binding protein found on HSV-1 and HSV-2. HSV-1 gD interacts with both the Herpes Virus Entry Mediator (HVEM—aka, HVEA/HveA, TNFR14) and nectin-1 (aka, HVEC/HveC). Non-human-infecting virus homologs of gD also have been identified in several species (e.g., swine, cows, dogs, etc.). Although level of sequence identity between such homologs are relatively low (e.g., -23%), functional overlaps between family members exist, including competition for the same receptors (e.g., nectin-1). See, e.g., Connolly S A, Whitbeck J J, Rux A H, et al. Virology. 2001; 280(1):7-18. Even among alphaherpesviruses that infect the same species there are significant differences in terms of sequence identity, suggesting that even sequences with differences in composition may retain/exhibit somewhat similar functions.

Herpes gD proteins have been a target for vaccine development, including in proposed DNA vaccines. See, e.g., Hensel et al., Journal of Virology April 2017, 91 (9) e02257-16 (combining gD vaccines with CpG oligonucleotides), U.S. Pat. No. 9,795,658 (teaching DNA vaccines encoding gD proteins, such as PEST sequences or ubiquitin sequences), and US20020058021 (combining gD, gB, or gC-encoding sequences with rabbit intron sequences and a tPA signal sequence in a DNA vaccine).

Researchers from the Wistar Institute have invented nucleic acid constructs using gD/antigen fusion proteins as a vehicle for antigen delivery (see, U.S. Pat. No. 8,962,816 (“the '816 patent”). Expression of DNA constructs described in the '816 patent reportedly results in one or more antigens being contained in a fusion protein containing an antigenic sequence inserted into the C-terminus (most broadly between residues 230-300) of a full length, truncated, or modified (mutated) gD protein, which retains the ability to interact with HVEM and possibly exhibit other gD functions (See, e.g., col. 8, lines 59-67). Unlike vaccine approaches, however, the '816 patent stresses the importance of the gD protein to interact with the HVEM cell receptor and to disrupt the HVEM-BTLA pathway, and thus act as a checkpoint inhibitor. The '816 patent specifies that the antigenic sequence of such a construct is fused directly to an N-terminal gD amino acid and to a C-terminal gD amino acid (See, e.g., col. 8, lines 11-25) and the experimental data reported in the '816 patent indicates that the inventors perceived this configuration of the fusion protein to be important to the enhanced immunogenic properties of such proteins compared to corresponding antigen-only constructs.

Although the '816 patent refers to a variety of possible antigens that can be contained in such a gD fusion protein-expressing construct, the '816 specifically exemplifies the use of only two types of gD fusion proteins. The 1^{st} construct comprised an HIV gag antigen embedded within the gD sequence. The 2^{nd} construct comprised an antigenic sequence that comprised a combination of apparently directly fused HPV E5/E6/E7 oncoproteins or just the E7 oncoprotein. A 3rd construct, at least designed to express a gD fusion protein comprising Thrombospondin-related anonymous protein (TRAP) “from parasite Plasmodium falciparum and Myco bacterium tuberculosis epitope string (TB) without their start and stop codons are incorporated into the HSV-1 gD NarI site, which corresponds to amino acid 288 in the gD mature form,” was specifically described, but not tested.

The inventors of the '816 patent administered the two above-described specific constructs to cells and animals as both plasmid and adenoviral constructs. While both types of vectors were sometimes able to induce an immune response (including a memory immune response lasting at least one year), the adenoviral vector constructs outperformed the plasmid vectors in most cases; in some cases the plasmids were deemed non-immunogenic (see, e.g., col. 34, lines 19-23); and the researchers ultimately appear to have envisioned that adenoviral vectors would be a required part of a clinical trial program (albeit possibly aided by initial plasmid immunization, a course that does not appear to have been employed in later related work).

In experiments reported in the '816 patent, T cell responses were induced by the two tested constructs in a variety of models and that, at least in some cases, measurable B cell (humoral) responses could also be induced for at least some of these constructs (e.g., vectors expressing the gD-gag fusion protein (see, e.g., Example 9)). Some of the gD fusion protein constructs described in the '816 patent, however, did not result in a measurably enhanced immune response when tested (see, e.g., Example 10), indicating that the specific gD/antigen/vector combination may be determinative of clinical efficacy, though the amount of guidance the '816 patent provides on this point is limited. As such, while the '816 patent demonstrated that gD fusion proteins can sometimes aid in inducing a B cell or T cell immune response to an incorporated antigen, the successful application of such a strategy to any particular gD-fusion protein context will continue to require ingenuity. Furthermore, no effort was made to measure innate immune responses, however, or to include separate sequences for simultaneous induction of different arms of the immune system.

The named inventors of the '816 patent (Hildegund Ertl, Marcio O. Lasaro, and Luis Ferreira) have authored/co-authored several published research papers detailing experiments either using corresponding or similar gD fusion protein-expressing constructs (either as DNA vaccines or adenoviral vectors) containing multiple HIV or HPV antigens, usually involving 2-4 dosages of such constructs and corresponding measurements of T cell or both T cell and B cell responses in mice, typically reflecting improved immune responses, which the researchers consistently attribute to gD:HVEM binding and to placement of antigens within gD as described in the '816 patents. See, e.g., Lasaro et al., Microbes and Infection 7 (2005) 1541-1550; Lasaro et al., Nature Medicine. Vol. 14(2): 205 (2008); and Lasaro et al. (2009), supra).

An extension of this work, described in Santana et al., PLoS ONE 8(8): e71322 (2013), reviews the use of bicistronic plasmid vectors encoding 2 different gD fusion proteins with antigens from HPV, HIV, and Herpes Simplex Virus (HSV), with 20% of mice treated with one such vector remaining tumor free and 60% developing a therapeutic response against transplanted tumor cells. The article reports the multi-cistron approach led to a more efficient T cell response but admits there may have been differential expression from the 2-cistron construct of the different fusion proteins. The authors emphasized the importance of gD membrane localization to achieving such effects.

In 2014 Dr. Ertl and colleagues published a paper detailing various experiments involving the initial and booster administration of gD fusion protein-expressing adenoviral vector constructs comprising the Epstein-Barr virus nuclear antigen 1 (EBNA-1) in Rhesus Macaques. Although the researchers saw a marked increase in EBNA-1 specific CD8 and CD4 cells receiving the gD:EBNA-1 fusion protein-expressing construct, the differences in animals receiving the fusion protein treatment versus just the antigen were “too subtle to clearly demonstrate an effect of HSV gD,” despite the previously obtained results with different constructs in mice. While the researchers also did not detect a difference in T cell response between animals administered a HVEM-binding vs. non-HVEM-binding form of gD, also contradicting early work with gD fusion protein-expressing constructs, the authors nonetheless noted that “only animals that received the vaccine with [HVEM] binding gD developed increased CD8EM responses that were significantly larger” than responses from unrelated antigen-only administration.

In additional follow-on research conducted by Dr. Ertl, a nucleic acid sequence encoding a combination antigen named “Melapoly” (comprising unspecified spacer-separated sequences of CD4+ and CD8+ T cell epitopes of melanoma-associated Ags (MAAs) including tyrosinase-related protein (Trp)-1, Trp-2, gp100, and mutated BrafV600E linked to the universal TH cell epitope PADRE, and an endoplasmic reticulum (“ER”) targeting signal sequence) was inserted into a gD-encoding sequence in the manner of the '816 patent constructs, and the sequence was delivered to and expressed in animals in a mouse melanoma model via chimpanzee adenoviral vectors similar to those used in the '816 patent (Zhang Y and Ertl C J (2014). The Effect of Adjuvanting Cancer Vaccines with Herpes Simplex Virus Glycoprotein D on Melanoma-Driven C D8+ T Cell Exhaustion. J. Immunol. 193(4): 1836-46). This research found that the gD-Melapoly fusion protein was able to induce T cell immune responses, however the response was not always clearly stronger than that achieved with immunization of antigen alone. What the researchers noted, however, was that the results of the reported experiments indicated that such a gD-T cell antigen construct might be capable of inducing a more effective T cell response in animals under a condition of T cell exhaustion, which results from continued antigen stimulation of T cells in chronic viral diseases (e.g., lymphocytic choriomeningitis virus, HIV, and HCV infection) or related (“subsequent”) cancers. The authors also highlighted the focus of these products on stimulating only CD8+ T cells as an advantage of such GD fusion protein constructs (see p. 1845, right col., second full paragraph).

Experiments involving gD-Melapoly-expressing adenovirus nucleic acid constructs are also described in U.S. Pat. No. 9,744,424 (to Ertl and Zhang). The '424 patent further discloses the combined uses of adenoviral vectors of gd:Melapoly-encoding adenoviral vectors with fibroblast activation protein (“FAP”)-encoding adenoviral vectors, either concurrently or in a prime boost administration strategy. The '424 patent provides structural information relating to the Melapoly multi-antigen sequence, reflecting that it contains three CD4+ T cell epitopes and eight CD8+ T cell epitopes from four melanoma associated antigens (see Cols. 21-22) separated by unspecified “conventional linker sequences” (presumably alanine-alanine spacer sequences). While significantly enhanced CD8+ T cell responses were reported to be induced in mouse tumor model animals treated with the gD:Melapoly-expressing adenoviral vectors, the composition failed to cure mice completely, especially in mice with advanced tumors (see col. 24, lines 15-22). Results were improved when the two different adenoviral vectors were co-administered. The addition of the FAP-expressing vector appears to have resulted in detection of T cells to FAP epitopes, a significant increase in certain cytokine associated T cells, and a low antibody response at the highest level of vector administration.

Additional related work has been more recently developed by researchers from the Wistar Institute, including Dr. Ertl, in collaboration with the present applicant, MBF Therapeutics. One set of these experiments, described in Kurupati et al., Cancer Immunol Immunother. 2018 October; 67(10):1533-1544, evaluated the ability of adenoviral vector comprising a DNA sequence encoding one of two canine cancer antigens (tyrosine-related protein 1 (Trp-1) or canine tyrosine-related protein 2 (Trp-2)) in a gD fusion protein to generate T cell immune response in dogs. Kurupati et al. indicated that the selected adenoviral vector was used because of its own very high immunogenicity. A T cell response was observed for the Trp-1/gD adenoviral constructs in dogs (responses for the Trp-2/gD constructs were not measured). Otherwise, Kurupati et al did not extend the '816 patent's teachings.

In still another set of even more recent experiments supported and/or performed by the same entities (MBF and Wistar), an adenoviral vector comprising an a gD fusion protein (gD FP) encoding sequence (ES) (gDFPES) construct similar to those described in the '816 patent, but encoding a fusion protein called K9Melapoly that comprised a combination of five predicted immunogenic amino acid sequences (comprising a set of predicted T cell epitopes from both Trp-1 and Trp-2 canine melanoma tumor-specific antigens along with human cancer-related immunogenic proteins tyrosinase, gp100, and MAGE-A1, in a fusion protein along with gD domains, where the antigenic sequences were separated by alanine-alanine (“A-A”) spacers, apparently similar to the approach taken in Zhang and Ertl, supra). This work is described in WO2020072371. The work described in WO2020072371 concludes that the GD-K9Melapoly fusion protein-encoding adenoviral vector was able to induce CD8+ T cell responses. In some respects, the vaccine also exhibited CD4+ effects, but in other cases such effects were “marginal” or “low,” which the researchers attributed to the selection of epitopes that target MHC Class I molecules. Three dogs tested with the vaccine produced certain, robust CD8+ T cell responses, but almost a similar number of dogs (2) were considered low responders. CD4+ responses, as noted were low or, at best, comparable with respect to only CD95+CD4+ cells. The tested and in-detail described construct was not designed to induce humoral, innate/adaptive, or innate immune responses, and, accordingly, such responses were not measured in the study.

Despite the promising results reflected in the '816 patent and various related publications and patent documents, in the period since counterparts of the '816 patent were published, the development of gD fusion protein treatments for cancer or viral diseases has remained remarkably limited, with apparently no known human clinical trials of such products initiated yet, and few publications directed to research involving such constructs other than those cited in this disclosure.

Also, despite significant advances in immunology, T cell vaccine products generally have often failed to result in clinically meaningful responses. For example, Gilbert, Immunology. 2012 January; 135(1): 19-26, reports “Although many ways of inducing T cells by vaccination have been assessed, the majority result in low level, non-protective responses” and only one on-market vaccine as of 2012 was thought to work primarily through T cell immunity. Even using multiple-antigen viral vector vaccine constructs developed by leading pharmaceutical company Merck have failed to deliver results in phase II clinical studies. Id. Results with adenovirus, pox virus, and other viral vectors have appeared more promising than with DNA vaccines. Id. However, as acknowledged by even the '816 patent, viral vector-based immunization approaches can be associated with undesired immune responses, rendering such products unsuitable for therapeutic application. E.g., Gilbert, 2012, supra, reports that p53-expressing viral vectors led to immune responses against the viral vector, rather than p53, in most patients.

Further, despite the extensive amount of research reported in the Wistar Art, relatively few other researchers have studied or reported on biotechnology applications of gD fusion proteins or related constructs. US 20030236396, which names a group of inventors apparently associated with University of Lausanne, University of Pennsylvania, and leading healthcare company Becton Dickinson, describes generation of truncated HSV gD polypeptides (e.g., comprising amino acid residues 1-337 of HSV-1 gD), and noting the ability of such polypeptides to promote glycosylphosphatidylinositol (GPI) anchoring, secretion, and other properties. Most of the '396 application's disclosure focuses appears on the use of such gD sequences for the development of polypeptide expression products, which can be cleaved away from the gD sequence after expression. Although Examples 12 and 13 describe gD fusion protein constructs, these constructs appear to have been made to demonstrate the secretion-promoting abilities of such gD sequences, as Example 13 is focused on the cleavage of gD from the non-gD portion. Example 14 discloses prophetic use of constructs encoding such gD sequences in the context of restorative gene therapy, but no examples or disclosure are provided describing or suggesting the use of such constructs as vaccines or immunogenic agents. As such, the '396 application appears directed to a fundamentally different approach than the Wistar Art. Further, the '396 application was abandoned prior to any substantive examination, no related patent applications were filed, and no related publications appear to exist in the literature, suggesting that these institutions did not view such ideas to be promising enough to be developed further or that the actual results associated with the specific constructs did not work as intended.

Other reports of gD fusion proteins in the literature have been primarily, if not exclusively, focused on understanding the basic biology of gD/HSV, rather than in the development of compounds for use as vaccines or immunotherapeutic agents (Zhou and Roizman. 2007. PNAS USA. 104:4142-4146. 10.1073/pnas.0611565104; Zhou et al. 2002. Proc. Natl. Acad. Sci. U.S.A 99:15124-15129. 10.1073/pnas.232588699; & Menotti et al. 2008. J. Virol. 82:10153-10161. 10.1128/JVI.01133-08).

Complicating the predictability of gD related constructs further is fact that much of the research conducted with gD fusion proteins to date has been in mice, and a growing body of scientific research has demonstrated that success with one treatment in one species is often not able to be successfully extended to other species, and this is particularly true in the case of translating mice data to larger animals. See, e.g., Akhtar, Camb Q Healthc Ethics. 2015 October; 24(4): 407-419. Mak et al. report that successful translation from animal models to human data in cancer treatment, specifically, has been less than 8%, which the authors attribute in part to the prevalent use of mice models. Am J Transl Res. 2014; 6(2): 114-118. Hayden, 2014, Nature News (available at Nature.com/news/misleading-mouse-studies-waste-medical-resources-1.14938). The challenge of translating success in mice to other animals has already demonstrated to have occurred with gD fusion protein-expressing constructs in at least one case, as noted by Ertl et al. (2014), supra. As such, the efficacy of such constructs in other animals remains unclear, and this is especially true in the case of animals such as pigs and cows in which HVEM does not appear to be expressed by host cells. Also, the predictability of success in therapeutically modulating immune systems with new nucleic acid vaccine constructs even in constructs similar to those that have exhibited immunogenicity in mice studies still often remains relatively low.

In view of these facts, successful development of products that can improve on the performance of immunogenic constructs known in the art will clearly require the application of inventive ingenuity.

Principles of Construction & Associated Abbreviations

Any heading(s) here (e.g., “Principles of Construction . . . ”) are used for convenience and do not limit the scope of the invention. Uncontradicted, aspects described under any heading/section can combined or applied to any aspects described elsewhere herein.

Terms such as “understood,” “in the art,” “readers,” “known,” “ordinary meaning,” and “skilled persons” refer to one of ordinary skill in the art or the knowledge thereof. The term “uncontradicted” means not contradicted by this disclosure, logic, or plausibility based on knowledge of skilled persons.

Uncontradicted, any description of terms already known in the art is for exemplifying aspects only; and is not intended to limit the scope of any aspect. Uncontradicted, all technical and scientific terms used herein have the same meanings as commonly understood by one of ordinary skill in the art and implicitly comprise the broadest interpretation based on such usage as well as any narrower interpretation(s) based on specific descriptions provided here. In general, any methods and materials similar or equivalent to those described herein can be used in the practice or testing of embodiments of the invention, the methods, devices, and materials described herein.

The inclusion of “(s)” after an element indicates that ≥1 of such an element is present, performed, and the like. E.g., a composition comprising NAM(s) comprising NS(s) means a composition including one or more NAMs collectively comprising one or more NSs.

For conciseness, symbols are used where appropriate. E.g., “&” is used for “and” & “˜” for “about.” The indicator “+” can be used with a value to indicate “or more than” (e.g., “1+ NAM” means “≥1 NAMs”). Symbols such as <and ≥ are given their ordinary meaning (e.g., “≤” means “less than or equal to” & “≥” means “equal or greater than”). A slash “/” can indicate “or” (A/B means A or B) or synonymous names of an element.

Ranges of values are used to concisely refer to each value falling within the range within an order of magnitude of the endpoints of the range without having to explicitly write each value. E.g., a recited range of 1-2 implicitly discloses each of 1.0, 1.1, 1.2, . . . 1.9, and 2.0 and 10⁻²⁰ implicitly discloses each of 10, 11, 12, . . . 19, and 20). Uncontradicted, all ranges include the end points, regardless of how the range is described. E.g., “between 1-5” includes 1 and 5 in addition to 2, 3, and 4 (and all numbers between such numbers within an order of magnitude of such endpoints, e.g., 1.1 and 4.9).

Terms of approximation (e.g., “about” or “approximately”) are used to conveniently refer to a range of closely related values or where a precise value is difficult to define. Uncontradicted, all exact values provided herein are representative of corresponding approximate values and vice versa (e.g., disclosure of “about 10” is to be understood as also providing support for 10 exactly and vice versa). Ranges described with approximate numbers include the recited endpoints and other relevant values encompassed by each endpoint regardless of presentation (e.g., “about 10⁻²⁰” should be interpreted in the same manner as “about 10—about 20”). The scope value(s) modified by terms of approximation will depend on the context of the disclosure or understanding of those skilled in the art. In the absence of such guidance, terms such as “about” should be understood as meaning +/−10% of the indicated value(s).

Lists of elements are sometimes employed for conciseness. Unless indicated, each member of each list of aspects or features should be viewed as an independent aspect of the invention. Each such aspect can have and often will comprise nonobvious properties with respect to the other listed elements.

Uncontradicted the terms “a” and “an” and “the” and similar referents are to be construed to cover both the singular and the plural. Terms in the singular implicitly convey the plural and vice versa herein, unless clearly contradicted by context or plausibility (e.g., a passage referring to use of a “composition” implicitly discloses corresponding use of corresponding “compositions,” and vice versa).

Terms such as “here” & “herein” means “in this disclosure” unless otherwise indicated. The term “i.a.” (“ia” or “ia”) means “inter alia” or “among other things.” “Also known as” is abbreviated “aka.” The term “elsewhere” means “elsewhere herein.” “SFE” means “see, for example.”

Uncontradicted, the term “some” regarding elements means “two or more” and regarding a part of a whole means “at least 5%” (i.e., ≥5%). The abbreviation OSMGAOA means “one, some, most, generally all (i.e., at least 75%), or all,” each of which is an independent aspect.

The modifier “DOS” means detectable or significant/detectably or significantly. “Significant” means results that are statistically significant using an appropriate test in the given context (e.g., p≤0.05/0.01).

“AAW” means “associatively applied with.” A composition or method is AAW another method/step or composition/element when the application results in DOS enhanced clinical effects (CEs), physiological effects, and the like. AAW can comprise co-administration/application, sequential administration/application, or both, ≥1 times. “AW” means “associated with.”

Use of the term “or” herein is not meant to imply that alternatives are mutually exclusive unless clearly stated or clearly contradicted by context. Thus, uncontradicted, “or” means “and/or.” The occasional explicit use of “and/or” herein has no effect on this interpretation of “or.” The scope of “or” meaning “and/or” in a phrase such as “A, B, and/or C” implicitly supports each of the following embodiments: A, B, and C; A, B, or C; A or C; A or B; B or C; A and C; A and B; B and C; A (alone); B (alone); and C (alone). The term “also” means “also.” Terms such “and combinations,” or “or combinations” regarding elements means combinations of any or all thereof. Use of the abbreviation “etc.” (or “et cetera”) in association with a list of elements means any or all suitable combination of the elements or any equivalents of such recited elements for performing any function(s) or step(s) in the art.

Terms such as “including,” “containing,” and “having” should be interpreted openly herein, e.g., as meaning “including, but not limited to,” “including, without limitation,” or “comprising,” unless the description clearly states otherwise. Comprising means including any detectable amount of an element or including any detectable performance of a step. Description of an aspect “comprising” or “including” a step or any element should be interpreted as also including that element/step (with any other steps/elements or alone).

Uncontradicted, a description of aspects including element(s)/step(s) using terms such as “comprising” or “including” implicitly discloses corresponding aspects that (1) consist of the step(s)/element(s) (or “is” the element(s)/step(s)), (2) consist(s) essentially of the step(s)/element(s), (3) substantially consists of the step(s)/element(s), (4) generally consists of the step(s)/element(s) (or is “generally adapted” to, is “generally composed” of, “generally is,” “generally only” is/are, “generally are,” the element, and the like), (5) predominately comprises (“mostly” or “primarily” comprises) the step(s)/element(s), (6) materially comprises the step(s)/element(s), or (7) appreciably comprises the step(s)/element(s).

The phrases “consists of” (sometimes abbreviated “CO”) and “consists essentially of” are understood in the art. “Substantially consists of” (“SCO”) means ≥95% of the referenced collection, effect, etc., is made up of the referenced element and “substantially associated” means that at least 95% of a referenced item are associated with a second referenced item. “Substantially all” means at least 95% of the referenced items/steps meet(s) the indicated condition. Phrases/terms such as “generally consists of” (abbreviated “GCO”), “generally is,” “generally are,” “generally all,” “generally,” or “generally is composed of” means referenced element(s) makes up ≥75% of the related whole. Similarly, the phrase “generally associated” means ≥75% of an element is associated with a 2nd referenced item (e.g., ≥75% of 1 agent is associated with a 2nd agent). Phrases such as “generally most” and “generally all” mean ≥75% of the referenced items/steps meet indicated condition(s).

“Predominately comprises” (abbreviated “PC”) means that detectably greater than 50% of the referenced collection, effects, etc., are composed of or attributed to the referenced element(s)/component(s) and “predominately associated” is construed similarly. “Materially comprises” (“MC”) means ≥5% of the composition/component is made up of the subject element/component. The phrase “materially associated” is similarly construed. The phrase “in material part” means ≥5% of the referenced element(s)/steps meet condition(s). “Appreciably comprises” means at least 1% of the composition is composed of the referenced element/component.

Changes to tense or presentation of terms (e.g., using “comprises predominately” in place of “predominately comprises”) should not be interpreted as modifying the meaning of the related phrase unless indicated.

Except where explicitly indicated or clearly indicated by context, “improved” herein means “increased.” In aspects, “improved” means “reduced,” such as in respect of toxicity of a composition. Uncontradicted, terms such as “enhanced,” “improved,” and the like are used synonymously.

Known elements associated with a function can be described as “means for” performing a function in a composition/system or a “step for” performing a part of a method, and parts of this disclosure refer to “equivalents,” which means equivalents known in the art for achieving a referenced function or step. However, no element of this disclosure or claim should be interpreted as indicating a “means-plus-function” construction unless such intent is clearly indicated by use of the terms “means for” or “step for.” Terms such as “configured to” or “adapted to” do not indicate “means-plus-function” interpretation, but, rather, describe element(s)/step(s) configured to, designed to, selected to, or adapted to achieve a certain performance, characteristic, property, or the like using teachings herein and in the art.

Unless otherwise indicated, compositions specifying a percentage are by weight unless a different value would be understood to apply to the element. If a variable is not accompanied by a value, any previously provided value typically applies to it.

Uncontradicted, all methods provided here can be performed in any suitable order. Unless contradicted, elements of a composition can be assembled in any suitable manner by any suitable method. Uncontradicted, any elements, steps, components, or features of aspects and all variations thereof, etc., are within the scope of the invention.

Numerous examples of aspects are provided to illuminate the scope of the invention. Uncontradicted, any aspect can be combined with any other aspect. The breadth and scope of the invention should not be limited by any of the exemplary embodiments. No language here indicates any element is essential to the practice of the invention unless as much is explicitly stated.

All references (e.g., publications, patent applications, and patents), cited herein, are hereby incorporated by reference as if each reference were individually and specifically indicated to be incorporated by reference and set forth in its entirety herein. Disclosure of such documents relating to principles, methods, and compositions (PMCs) can be combined with the teachings provided herein to provide additional useful compositions and applications. However, citation/incorporation of patent documents is limited to the technical disclosure thereof and does not reflect any view regarding validity, patentability, etc., thereof. In the event of any conflict between this disclosure and the teachings of such documents, the content of this disclosure controls regarding aspects of the invention. Numerous references are cited here to concisely incorporate information and to aid skilled persons in putting aspects of it into practice. While efforts have been made to include the most relevant references for such purposes, readers will understand that not every aspect of every cited reference will be applicable to the practice of the invention.

### I. Additional Terms and Acronyms Used in this Disclosure

The following description of certain terms and acronyms is provided to assist readers in understanding the invention. This section not intended to limit the scope of any terms ordinarily understood in the art. As such, any term description is generally to be viewed as describing aspects or exemplifying scope of the term. Additional terms and abbreviations are provided in other parts of this disclosure, are known in the art (e.g., DNA), or both (e.g., CpG).

A “biomolecule” here typically means a composition comprising, PC, GCO, or CO a PPT or NAM. In aspects, a biomolecule can include other molecules made by cells/synthetic counterparts, e.g., lipids & carbohydrates.

Terms like “inducing” means DOS inducing, promoting, or enhancing an event/outcome, such as IR(s) (generally), particular IR(s) (e.g., ITIC IR(s)) or T cell IR(s)), or CE(s), e.g., reduction of symptom(s). “Enhancing” comprises increasing the magnitude, scope, duration, or other characteristic, typically of another event or composition. “Promoting” means increasing the likelihood of occurrence, frequency of occurrence, etc.

Terms such as “block,” “inhibit,” or “reduce” herein, e.g., with respect to IR(s), CE(s), checkpoint pathway(s) (CP(s)), or other physiological or cellular processes means reduces, attenuates, stops, makes less likely, or prevents as dependent on context—e.g., a biomolecule that “blocks” a checkpoint pathway reduces, inhibits, or stops such a checkpoint pathway from its typical operation in the given context. Both such effects (inducing and reducing) mean at a detectable level and implicitly comprise at a statistically significant (“significant”) level.

“Peptidic” means primarily or only composed of AARS(s).

“CCEPM” means complementary clinical event-promoting method. A CCEPM is a method that when AAW delivery of CCEPCs induces CEs. E.g., with respect to treating cancer, CCEPMs include surgery or radiation therapy.

“CCEPC” means complementary clinical event-promoting composition. CCEPCs are compositions that induce CEs when AAW complete expression product (EP) encoding sequence (ES) compositions (CEPESCs), including vaccines, therapeutics, and medicaments that treat symptoms.

Phases such as “applied in association with,” AAW, delivered in association,” or “administered in association” mean delivery or administration of 2+ methods, steps, compositions, or combinations in any suitable manner(s), including, unless clearly contradicted, co-administration and sequential administration, repeated administration or application, and the like. “Delivery” or “administration” implicitly means delivery/administration of an “effective amount” (“EA”) (an amount that is effective to produce the desired result, typically at a DOS level, often an amount that effectively induces IR(s) in a TR, in a population of TR(s), e.g., as determined through performance of 1-3 adequate and well-controlled clinical trial(s), or both (whether reference to EA(s) is made or not)). Terms like “deliver” refer to the transfer of a composition (e.g., a CEPESC) to a physiological site, tissue, cell, or to/into a TR. “Delivery” encompasses delivery to the intracellular portion of a cell or extracellular space(s). Delivery of a NAM into the intracellular portion of a cell is ORT as “transfection.” Delivery and administration are often used synonymously here. Description of elements of compositions herein, unless contradicted, also are implicitly to be understood as being present in EA(s).

Uncontradicted, PPTs described here with respect to a WT protein, class of proteins, etc., encompass both PPTs consisting of (CO) the AARS of the reference protein(s) and also FPs comprising the AARS, related AARSs (in terms of identity or similarity) and heterologous AARS(s). E.g., an “EAT-2 PPT” provides simultaneous support for PPTs that (1) have an AARS consisting of a WT EAT-2 sequence (which may be an entire WT EAT-2 PPT or an FF) or a FV or (2) including any such sequence and additional AARS(s).

Two sequences (nucleotide or AA (amino acid)), such as 2 sequences in a single recombinant molecule that have different origins, compositional characteristics, or both, can be described as “heterologous” (relative to each other). Conversely, sequences that share origin(s) or compositional characteristics are “homologous.” In WT PPTs and NAMs, origin is the sole determinant of homology herein. Origin in such respects refers both to genes and gene products. Homologs include PPTs recognized as homologous ITA based on functional, structural, and compositional characteristics (e.g., human EAT-2 and murine EAT-2; PRV gD and HSV-1 gD; canine influenza HA and human influenza HA; etc.). However, with respect to variants (FVs), compositional similarity to a WT biomolecule is the determinant of homology. E.g., a FP comprising a functional variant/variation (FV) of a human EAT-2 and a variant of an HSV-1 gD would be heterologous given that each AARS is related to PPTs from different species.

The modifier “EL” means “entire length” and can refer to wild-type (WT). E.g., an EL HSV-1 gD means AAs 1-394 of HSV-1 gD.

An immunomodulator (“IM”) is a composition/biomolecule that modulates IR(s) (e.g., that induces IR(s) or block/inhibit IR(s), typically in a non-Ag specific manner. An “antigen” (“Ag”) is a biomolecule or part thereof that induces antigenic response(s) in cells/subjects. Antigenic PPTs (Ags) are distinguishable from IMs or adjuvant(s). While Ags induce IR(s) to the content of the Ag, IMs and adjuvant(s) induce IR(s) generally. However, readers will recognize such terms can overlap in scope and that, in aspects, Ags can be PIMs & vice versa. In aspects Ag(s) are PIM(s) & in many aspects some, most, generally all, or all (SMGAOA) Ag(s) in CEPs are not IM(s).

The term “exogenous” can be used to indicate a referenced molecule or the referenced activity that is introduced into a non-native context. The term “endogenous” is usually used to refer to a referenced molecule or activity that is present or typically present in a context.

The term “construct” means a recombinant nucleotide sequence (“NS”) comprising ≥1 PPT coding sequence(s) and typically is used synonymously with expression product encoding sequence (“EPES”).

The abbreviation RVRHRSIOI means “related, very related, highly related, substantially identical, or identical” with respect to two NSs or AARSs. RVRHROSI is construed similarly except for excluding identical sequences. Functional variant (“FV”) sequences can be related, very related, highly related, or substantially identical (RVRHROSI) or, in the case of FV AARSs both RVRHROSI & SVSHSOCE to WTC(s). “SVSHSOSCE” means similar, very similar, highly similar, or substantially compositionally equivalent.” Identity/relatedness and similarity of sequence(s) is defined by optimally aligning sequence(s) using a tool such as BLAST, LALIGN, or CLUSTAL using appropriate settings. “Related” means at least 70% identical, very related (VR) means ≥80% identity; highly related (HR) means ≥90% identity or no more than 2 differences in composition in sequences with less than 10 components; substantially identical (SI) means ≥96% identical or no more than 1 difference in sequences with less than 1 component. Description of a “related” sequence also implicitly discloses corresponding aspects CB VR, HR, or SI with respect to the related sequence. Similar (S) sequences (AARS(s)) exhibit ≥75% AARS similarity; very similar (VS) AARSs are ≥85% similar; highly similar (HS) AARSs are ≥92% similar; and [substantially] compositionally equivalent (CE) AARSs are ≥98% similar. “MCRT” means “most closely related to,” which is defined in terms of relatedness unless specified. Uncontradicted, disclosure of any PPT implicitly includes suitable FFs & FVs thereof. E.g., a reference to “PPT X” means “PPT X, an FF, or FV of either thereof.”

FVs and FFs can exhibit suitable, comparable, or improved function(s) compared to reference(d) WTC sequence(s)/biomolecule(s) (PPT/NAM). “Comparable” functionality means functionality within +/−15%, 10%, or 5% of the WT function. “Suitable” functionality means exhibiting/retaining function(s) that are not significantly different from the referenced counterpart function. In aspects, “suitable” functionality means at least 50% of the function of the WT counterpart. “Improved” functionality (improvably functional) means significant improvements in function(s) or an improvement of ≥20% of the function of the counterpart. Disclosure of any function herein implicitly discloses all such levels of functionality.

Terms such as “treat” or “treatment” generally mean to DOS reduce, stop, inhibit, alleviate, or ameliorate, the underlying cause of physical symptoms of disease-causing agent associated diseases (“DCAADs”), and encompass, in aspects, “curing” a DCAAD (eliminating detectable indication(s) of the DCAAD in TR(s)). Terms like “prevent,” “prophylaxis,” “immunize,” and “vaccinate” mean to induce IR(s) that are DOS protective in TR(s) when subsequently challenged with an associated DCA and encompass reduction in the likelihood of acquiring, duration, extent, or severity of a DCAAD, each as an aspect, and complete prevention (lack of detectable disease). Described here are other aspects characterized as methods of treating or preventing DCAADs.

The descriptor “anti-” herein typically means blocking, reducing, reversing, or diminishing. E.g., an “anti-cancer composition” is a composition that has one or more of such effects on a cancer condition in TRs. In connection with Abs and ligands, the prefix “anti” typically describes an antibody (“Ab”)/ligand that binds (and typically specifically binds) a referenced target.

In aspects, molecules, such as polypeptides and nucleic acid molecules are described as, i.a., “isolated.” Isolation and isolation methods are known in the art with respect to nucleotide sequences (NSs) (e.g., by hybridization) and peptides (e.g., by affinity chromatography, ELISA, SDS-PAGE, and the like). The term “isolated” with respect to a NS usually refers to a polynucleotide that is not immediately contiguous with both of the coding sequences with which it is normally immediately contiguous in its original environment (on the 5′ end, on the 3′ end, or both). Isolated polynucleotides include recombinant polynucleotides maintained in heterologous host cells or purified (partially or substantially) polynucleotides in solution. Isolated RNA molecules include in vivo or in vitro RNA transcripts of the polynucleotides of the present invention. Synthetic biomolecules also are typically isolated.

### Table of Commonly Used Acronyms

The following table lists acronyms that are frequently used in this disclosure and provides a description of the meanings/scope thereof:

| Acronym | Term | Brief Description |
|---|---|---|
| AA/AAR | Amino acid | An amino acid (aka, an AA residue) |
| AAC | Associative(ly) | A composition associatively applied with (AAW) |
| | applied composition | CEPESC(s) in methods |
| AARS | Amino acid residue | A sequence of amino acid residues (a part of a |
| | sequence | polypeptide (PPT)) |
| Ab | Antibody | An antibody molecule |
| AE | Adverse event | Any event reportable as an adverse event under |
| | | regulatory guidelines |
| Ag | Antigen | A biomolecule that induces an antigenic response |
| | | with respect to DCA(s) |
| AgES | Antigen-encoding | A nucleotide sequence (NS) encoding antigen(s) |
| | sequence | (Ag(s)) |
| AgV | Antigen variant | A variant of a wild-type (WT) Ag |
| aHV, αHV, or | Alpha-herpesvirus | Terms used to refer to such viruses or biomolecules |
| a-HV, etc. | | of such viruses |
| BC | B-cell | An antibody-producing cell including a B cell |
| | | receptor (BCR) or classified as a BC |
| BCE | B-cell epitope | An epitope recognized by a B-cell (BC) |
| CaPNP | Calcium phosphate | Any type of Ca phosphate nanoparticle that |
| | nanoparticle | promotes transfection of NAVs |
| CC | Combination | A composition comprising CEPES NAM(s) and |
| | composition | one or more CCC(s) |
| CCC | Combination | A component of a CC that enhances immune |
| | composition | responses (IR(s)) of composition components or |
| | component | independent induces IR(s)/CE(s) |
| CE | Clinical effect | A clinically relevant effect in TR(s)—e.g., |
| | | reduction in frequency, symptoms, or severity; |
| | | delay of onset; increase in cure rate/cure, or other |
| | | favorable change(s) in clinical indicator(s) in TR(s) |
| | | (at an individual or population level) |
| CEP | Combined expression | All expression products (EP(s)) (i.e., peptides |
| | product | expressed from a CEPESC |
| CEPES | Combined EP ES(s) | NS(s) encoding a CEP |
| CEPESC | Complete EP ES | A composition including all referenced EPES(s) |
| | composition | and associated elements |
| CI | Checkpoint inhibitor | A molecule that acts as a checkpoint inhibitor in |
| | | TR(s). A “PCI” is a peptide CI |
| CM | Checkpoint modulator | A molecule that modulates a checkpoint pathway in |
| | | TR(s) |
| COE | Cell(s) of expression | Cells in which EP(s) in a CEPESC are expressed |
| CP | Checkpoint pathway | A term given its ordinary meaning |
| CPCR | Checkpoint pathway | A cell receptor that forms part of a checkpoint |
| | cell receptor | pathway |
| CPRL | CP receptor ligand | A ligand for a receptor in a CP |
| CPSTAP | CP signal transducing | An adaptor protein that modulates member(s) of a |
| | adaptor protein | checkpoint pathway |
| CRA | Clinically relevant | An AARS/PPT that exhibits DOS CE(s) in a |
| | antigen | context as proven through clinical testing in TR(s) |
| | | or a population of TR(s) |
| CS | Coding sequence | A PPT-encoding NS, expressible in TR(s) |
| CSAE | Cytokine syndrome | Cytokine-associated AE(s) including cytokine |
| | AE(s) | storm effects, cytokine syndrome AEs, & Vaccine- |
| | | Associated Enhanced Respiratory Disease |
| CSM | Co-stimulatory | A molecule that induces a stimulatory checkpoint, |
| | molecule | e.g., an ICRL of a co-stimulatory ICR (a concept |
| | | known ITA) |
| CTL | Cytotoxic T | A modifier associated with cytotoxic action(s) of |
| | lymphocyte/CD8 | CD8 T cells or an associated IR, such as a T cell |
| | | cytotoxic IR |
| DC | Dendritic cell | Cells recognized as DCs in the art |
| DCA | Disease-causing agent | A pathogen, cancer/cancer cell, or other disease- |
| | | causing agent treatable through induction of an |
| | | effective antigenic IR |
| DCAAD | DCA-associated | A disease/condition AW a DCA, injurious or |
| | disease | potentially injurious to TR(s) |
| DCR | DC receptor | A receptor expressed on dendritic cell(s) |
| DCUF | DC uptake facilitating | A modifier associated with AARSs that DOS |
| | | facilitate DC uptake of associated AARS(s) in FPs; |
| | | “UF” means “uptake facilitating,” e.g., ICUF means |
| | | “immune cell uptake facilitating” |
| DIV | De-immunization | A variation that reduces the immunogenicity or |
| | variation or variant | antigenicity of a PPT/AARS in a TS |
| EEI | Expression enhancing | An intron that enhances the expression of |
| | intron | associated EP(s)/CS(s) |
| EP | Expression product | A peptide or polypeptide (PPT) expressed from an |
| | | NS in a CEPESC |
| EPES | EP encoding sequence | A NS encoding EP(s); combinable with other terms |
| | (aka coding sequence | (e.g., PCIES means a PCI encoding sequence) |
| EPESNAM(s) | EPES nucleic acid | Nucleic acid molecule(s) (NAM(S) comprising |
| | molecules | expression product encoding sequence(s) (EPES(s)) |
| ES | Encoding sequence | A nucleotide sequence (NS) that encodes a |
| | | polypeptide (PPT) or AARS |
| ETS | Extracellular targeting | An AARS that targets a referenced target expressed |
| | sequence | externally on cell(s) |
| FF | Functional fragment | A fragment of a PPT or NS that exhibits a function |
| | | that is comparable, equivalent, or superior to |
| | | function(s) of WTC(s). |
| FL | Flexible linker | A linker amino acid sequence that is at least 33% |
| | | composed of glycine residues |
| FP | Fusion protein | A protein/PPT comprising two or more |
| | | heterologous AARSs, typically that each exhibit |
| | | distinct DOS functions |
| FV | Functional variant | A variant of a reference biomolecule/sequence that |
| | | exhibits ≥suitable function(s) vis-à-vis WTC(s) |
| gD | Glycoprotein D | A glycoprotein D protein (gDP) of an |
| | | alphaherpesvirus or a modifier used to refer to gD- |
| | | related PPTs/AARSs |
| gDAgFP | gD-antigen fusion | A FP comprising one or more gD sequences and |
| | protein | heterologous sequences |
| gDD/gDS | Glycoprotein D | An AARS of a gDP, or an FF or FV thereof |
| | domain/sequence | |
| gDP | gD polypeptide | a PPT comprising gDS(s) |
| gDSS | gD signal sequence | An AARS that is/comprises a WT gDSS, an FF of |
| | | such an AARS, or a FV of either |
| GSRAgV | Glycosylation site | A variant Ag in which potential or actual |
| | removal Ag variant | glycosylation sites, are removed |
| GSRV | Glycosylation site | FV(s) of AARS/PPT(s) where ≥1 known or |
| | removal variant | expected glycosylation sites are removed (also |
| | (variation) | describes such variation) |
| IC | Immune cell | Any cell of the immune system |
| ICITM | Immune cell internal | An ITM that significantly, primarily, generally, or |
| | target modulator | only exhibits effects in ICs |
| ICR | Immune cell receptor | A receptor that is abundantly expressed on ICs and |
| | | when modulated induces IRs. |
| ICRL | IC receptor ligand | A ligand for an immune cell receptor |
| ICSTAP | Immune cell STAP | A STAP that significantly, primarily, generally or |
| | | only exhibits effects in ICs |
| IM | Immunomodulator | A compound that DOS modulates IR(s) |
| IR | Immune response | A DOS immune response in TR(s) |
| IRV | Immunodominance | A variation in an Ag that reduces |
| | reduction variation | immunodominance |
| ISNS | Immunostimulatory | A NS that is inherently immunostimulatory, such as |
| | nucleotide sequence | an immunostimulatory CpG sequence |
| ITIC | Innate trained immune | A cell of the innate trained immune system, such as |
| | cell | NKCs and DCs |
| ITICITM | ITIC internal target | A PPT that modulates activity of an internal target |
| | modulator | in ITICs |
| ITICR and | ITIC receptor & | Receptors and ligands of ITICs, respectively |
| ITICRL | ITICR ligand | |
| ITICSTAPs | ITIC signal | STAPs that modulate ITIC ICRs, e.g., SAP and |
| | transducing adaptor | EAT-2 |
| | proteins | |
| ITIPIMs | Innate Trained | Peptidic immunomodulator(s) that predominantly, |
| | Immunity Peptidic | generally, substantially, or only induce significant |
| | Immunomodulators | IRs in ITICs |
| ITM/ITIM | internal target | A PPT that DOS modulates a target biomolecule |
| | modulator or IT IM | present in a cell's interior |
| ITS | intracellular targeting | An AARS that targets a referenced intracellular |
| | sequence | target (e.g., an organelle, a PPT in the nucleus or |
| | | other organelle, or an internal portion of a receptor) |
| MgD and | Modified gD/gDS, | A non-WT gDS not described in the Wistar Art |
| MgDS | respectively | |
| MHCIE | MHCI epitope | An epitope that is associated with (AW) MHCI |
| | | presentation |
| MHCIIE | MHCII epitope | An epitope AW MHCII Ag presentation |
| MSFL | Mid-sized flexible | A linker of ≥4 AARs in length and ≥33% composed |
| | linker | of Gly residues |
| MSL | Mid-sized linker | A linker of at least 4 AARs |
| NAM | Nucleic acid molecule | A nucleic acid molecule, typically nucleic acid |
| | | molecule(s) in CEPESCs |
| NASM | Non-antibiotic | A selection marker that is not an antibiotic |
| | selection marker | resistance marker |
| NAV | Nucleic acid vector | A nucleic acid vector |
| NFP | Non-fusion protein | A PPT comprised of an AARS of or that is RVRHR |
| | | or SI to a single WT PPT |
| NGD or NgD | “Non-gD” | A modifier describing PPTs/AARss heterologous to |
| | | gDS(s) (e.g., a NgDICRL is a non-gD ICRL) |
| NGDICRTS | Non-gD ICR targeting | An ICR TS that is heterologous to gD receptor |
| | sequence | binding domains (gDRBDs) |
| NHA | Non-human animal | TR(s) (hosts) other than human beings |
| NDISTR | Non-DCA | TR(s) (hosts/subjects) not in a state of checkpoint |
| | immunosuppressed | immunosuppression at the time of CEPESC |
| | target recipient | administration |
| NKC | NK cell | A natural killer cell |
| NLGSRV and | N-linked GSRV and | Variations/FVs in which N-linked glycosylation |
| NLGSRAgV | N-linked glycosylation | sites are removed through substitution or deletion |
| | site removal AgV | of AAs; NLGS means “N-linked glycosylation site” |
| NonCMIMP(s) | Non-Checkpoint- | PPT(s)/AARS(s) that are IM(s) and not a PCI, such |
| or NCMIMP(s) | Modulating | as a Paj/cytokine |
| | Immunomodulator | |
| | PPT(s) | |
| NS | Nucleotide sequence | Sequences of nucleotide bases, such as DNA or |
| | | RNA sequences |
| OCEP | Other composition | PPT(s) expressed from NS(s) in compositions other |
| | expression product | than CEPESCs |
| PAg/PCRA | Putative antigen/ | PPT/AARS predicted to be an Ag/CRA |
| | Putative CRA | |
| PAj | Peptidic adjuvant | An adjuvant that is a peptide or AARs |
| PE | Polyepitope | A polyepitope sequence or a modifier relating to |
| | | such sequences (e.g., a NS sequence encoding a |
| | | PE) |
| PFD | Profusion domain | A profusion domain of a gDP (WT/FV/FF) |
| PIM | Peptidic | A polypeptide (PPT) that is an immunomodulator |
| | immunomodulator | |
| PMC | Principles, methods, | Used to reference art that teaches principles, |
| | or compositions | methods, or compositions adaptable to or |
| | | combinable with aspects |
| polyUb | Polyubiquitin | A term given its ordinary meaning |
| PPT | Polypeptide | A peptide, polypeptide, or protein, including a |
| | | multimeric peptide or protein |
| SCM | Stimulatory CM | A molecule acts as a CM that stimulates immune |
| | | system activity in a TR |
| SCS | Self-cleavage site | A site acted on by a self-cleavage element (e.g., a |
| | | 2A PPT cleavage site) |
| SCUP | Strong constitutive | A promoter recognized as having all of these |
| | universal promoter | qualities in TRs, such as a CMV promoter or a |
| | | CAG promoter |
| STAPM | STAP modulator | A PPT that modulates a STAP or STAP pathway |
| | | (e.g., a PPT that blocks a STAP-ICR pathway); |
| | | e.g., an IC STAPM PPT that modulates a |
| | | STAP/STAP pathway. |
| TC | T cell | Self-explanatory/known in the art |
| TCE | T-cell epitope | An epitope recognized by a T-cell |
| TFA | Transfection- | An agent associated with a vector, such as a NAV, |
| | facilitating agent | that promotes uptake of the vector into cells, such |
| | | as CaPNPs |
| TH | T-helper/CD4 | A modifier to indicate T-helper/CD4 T cell or |
| | | associated IR—a CD4/TH immune response (e.g., |
| | | a MHCII response) |
| TH17TCE | Th17 T-cell epitope | A TCE that primarily, generally, or only induces |
| | | Th 17 cytokine responses |
| TH1TCE | Th1 T-cell epitope | A TCE that primarily, generally, or only induces |
| | | Th1 cytokine responses |
| TH2TCE | Th2 T-cell epitope | A TCE that primarily, generally, or only induces |
| | | Th2 cytokine responses |
| TMD | Transmembrane | An AARS that corresponds to or is a variant of a |
| | domain | WT transmembrane domain of a PPT, such as a |
| | | TMD of a WT gDP |
| TME | Tumor | The environment of cancer cells in and around a |
| | microenvironment | tumor including nearby non-transformed cells and |
| | | milieu |
| TR | Target recipient | Subject(s) intended to receive a referenced |
| | | CEPESC (e.g., a pig, dog, horse, or human) |
| TREGE | TReg epitope | An epitope that induces a TReg IR |
| TS | Targeting sequence | An AARS that binds a referenced PPT, e.g., an |
| | | ICR. E.g., a DCRTS is a DCR TS |
| WT | Wild-type | A modifier used to identify a naturally occurring |
| | | form of a PPT, cell, NS, biomolecule, organism, or |
| | | the like |
| WTC | Wild-type counterpart | A biomolecule/sequence that is a counterpart of an |
| | | FF or FV |

Common acronyms used below, such as longer acronyms which combine shorter acronyms in the table above, are additionally listed here:

| | |
|---|---|
| AbFP | Antibody (Ab) fusion protein |
| CDR | Complementarity determining region |
| CIMP | Checkpoint immunomodulator polypeptide |
| DCR | Dendritic cell receptor |
| DCRTS | Dendritic cell receptor targeting sequence |
| EPESNAM | Expression product encoding sequence NAM |
| ERTPS | Endoplasmic reticulum targeting/processing |
| | sequence |
| ESE | Exonic splicing enhancer |
| ETS | Extracellular targeting sequence |
| gDAgFP | Glycoprotein D antigen fusion protein |
| gDAgFPES | Glycoprotein D antigen fusion protein encoding |
| | sequence |
| gDFPES | Glycoprotein D fusion protein encoding sequence |
| gDP | Glycoprotein D protein |
| gDPES | Glycoprotein D polypeptide encoding sequence |
| gDRBD | Glycoprotein D receptor binding domain |
| gDSFP | Glycoprotein D sequence fusion protein |
| gDVS | Glycoprotein D variant sequence |
| HRSIOI | Highly related, substantially identical, or identical |
| ICTIM | Immune cell target immunomodulator |
| ICTS | Immune cell targeting sequence |
| IRES | Internal ribosome entry site |
| ITICICRTS | Innate trained immune cell immune cell receptor |
| | targeting sequence |
| ITICICRTSFP | Innate trained immune cell immune cell receptor |
| | targeting sequence fusion protein |
| MGAOA | Most, generally all, or all |
| MGASAOA | Most, generally all, substantially all, or all |
| NANCIPI | Non-checkpoint innate peptide immuno- |
| | modulator |
| NGDCI | Non-glycoprotein D checkpoint inhibitor |
| NGDICRTSFP | Non-glycoprotein D immune cell receptor |
| | targeting sequence fusion protein |
| NGDPCI | Non-glycoprotein D peptidic checkpoint inhibitor |
| NLGSRAgVS | N-linked glycosylation site removal Ag variants |
| NVNBO | Non-viral, non-bacterial pathogenic organism |
| NVNBM | Non-viral, non-bacterial pathogenic micro- |
| | organism |
| PCI | Peptidic checkpoint inhibitor |
| PCM | Peptidic checkpoint modulator |
| PEFP | Polyepitope fusion protein |
| PCGCOOCO | Predominantly comprises, generally consists |
| | of, or consists of |
| PCGCOSCO | Predominantly comprises, generally consists |
| | of, or substantially consists of |
| PCOGCOCO | Primarily consists of, generally consists of, or |
| | consists of |
| PCRAES | Putative clinically relevant antigen encoding |
| | sequence |
| PTPS | Proteosome targeting/processing sequences |
| RVRHR | Related, very related, or highly related |
| RVRHROSI | Related, very related, highly related, or |
| | substantially identical (used with respect to |
| | compare sequences) |
| RVRHRSIOI | Related, very related, highly related, |
| | substantially identical, or identical |
| S/MAR | Scaffold/matrix attachment region |
| SCS | Self-cleavage site |
| SMGAOA | Some, most, generally all, or all |
| SVSOHR | Similar, very similar, or highly related |
| SVSHSOCE | Similar, very similar, highly similar, or |
| | compositionally equivalent |
| RBD | Receptor binding domain (the modifier BD can |
| | refer to domains that bind named receptors— |
| | e.g., an HVEMBD |
| TAA | Tumor associated antigen |
| UbL or UBL | Ubiquitin-like |
| VRHROSI | Very related, highly related, or substantially |
| | identical |

### SUMMARY OF THE INVENTION

This invention provides new methods and compositions for inducing immune response(s) (IR(s)) in target recipient(s) (TR(s)).

These methods and compositions typically comprise/include delivering nucleic acid molecule(s) (NAM(s)) comprising constructs that include antigen (Ag) encoding sequences (ESs), and which constructs are associated with one or more additional elements that promote the uptake or processing of such nucleic acids, antigens, or both, thereby improving immune response(s).

Methods/compositions can be characterized in i.a., (a) the combined expression product(s) (CEP(s)) expressed from such combined expression product encoding sequences compositions (CEPESCs) comprising (a) novel Ag(s), Ag combinations, or antigen variant(s) (AgV(s)) (e.g., glycosylation site removal Ag variant (GSRAgV(s))), (b) Ag-associated TS(s) (e.g., intracellular targeting sequence (ITS(s)), e.g., proteosome targeting/processing sequence(s) (PTPS(s)), e.g., polyUb(s)), (c) peptidic immunomodulator (PIM(s)) (e.g., innate trained immune cell (ITIC) internal target modulator (ITM) (ITICITM(s))/innate trained immune cell signal transducing adaptor protein(s) (ITICSTAP(s)), (e.g., EAT-2 polypeptide(s) (PPT(s))) or other peptidic checkpoint modulator(s) (PCM(s)), e.g., trap proteins targeted to checkpoint pathway targets), (d) modified gD sequence(s) (MgDS(s)); or (e) combinations thereof; (2) nucleic acid molecule(s) (NAM(s)) comprising such combined expression product (EP) encoding sequences (ESs) (CEPESs) (a) comprising expression enhancing intron (EEI(s)); (b) being nucleic acid vectors (NAVs) (e.g., mRNA NAV(s), mixtures of NAV(s), NAV(s) comprising non-antibiotic selection marker(s) (NASM(s)), etc.); or (c) being associated with transfection facilitating agent(s) (TFA(s)), e.g., calcium phosphate nanoparticle(s) (CaPNP(s)) that enhance/induce immune response(s) (IR(s)); or (3) treated subjects (TR(s)) (a) being non-HVEM-expressing TR(s), (b) being TR(s) not undergoing active disease causing agent (DCA) immunosuppression through DCA-associated checkpoint modulation, (c) being at risk or undergoing a cytokine syndrome condition, (d) being treated with or at risk of treatment with a “leaky vaccine,” or (e) a combination. “Leaky vaccines” are described in US20200325182. EHV and PCV are exemplary leaky vaccine-associated disease-causing agents (DCAs).

In one exemplary aspect, compositions include, and methods comprise, delivery of sequences encoding glycoprotein D polypeptide (gDP(s)) and antigen(s) (Ag(s)), typically through the delivery of complete expression product (EP) encoding sequence (ES) composition(s) (CEPESC(s)).

Another facet is embodied in CEPESCs comprising NS(s), NAV(s), and TFA(s), e.g., as described elsewhere. Other facets relate to related compositions (e.g., expression product polypeptides (PPTs) and related compositions, cells comprising expression product (EP) encoding sequence(s) (ES(s)) (EPES(s)), kits, and packaged compositions, etc.), methods of production, related methods of use (e.g., use of CEPESCs comprising combination composition(s) CC(s), or associatively applied with (AAW) complementary clinical event promoting composition(s) (CCEPC(s)) or complementary clinical event promoting method(s) CCEPM(s)).

In aspects, gDP(s) in compositions/encoded by constructs are gD fusion proteins (gDFPs) comprising gD AARS(s) (aka, gD sequence(s) or (gDS(s)) sometimes comprising glycoprotein D (gD) domain(s) (gDD(s)) and heterologous AARS(s). In aspects, heterologous AARS(s) comprise Ag(s) (“Ag” here can be used to refer to non-fusion protein (NFP) antigens(s) (e.g., endogenous Ag(s)) as well as to Ag(s) incorporated into fusion proteins (FPs)). Such gDFPs can be referred to as glycoprotein D (gD) antigen (Ag) fusion proteins (FPs) (gDAgFPs). In aspects, gDAgFP(s) or other FP(s) comprise internal targeting sequence(s) (ITS(s)) that are associated with Ag(s) (e.g., exosome TS(s), endoplasmic reticulum (ER) TS(s), etc.). In aspects, ITS(s) are proteasome targeting/processing sequence(s) (PTPS(s)). In aspects, PTPS(s) comprise polyUb(s). In aspects, gDAgFP(s) comprise Ag(s) downstream of any gDS(s) in the gDAgFP. In gDP(s), such as gDAgFP(s), gDS(s) can comprise entire length (EL) wild-type (WT) gDP(s), a functional fragment (an FF), or a functional variant (an FV) thereof. In aspects, defined gDS(s) of gDP(s) exhibit ≥1 defined function(s), such as gD receptor (gDR) binding, checkpoint inhibition, inducing ER processing, ensuring desired placement of other gDDs, etc. In aspects, a gDS is combined with ≥1 heterologous gDS(s) to generate a chimeric gDS that exhibits such gD function(s) (e.g., a chimeric gDRBD (gD receptor binding domain) that can bind gD receptor(s)). In aspects, FFs/FVs of gDS(s) or other referenced PPTs/AARSs/NSs exhibit suitable, comparable, or improved function(s) with respect to wild-type counterpart(s) (WTC(s)).

In aspects, functional fragments (FFs) are used/expressed in place of full length (FL)/wild-type (WT) polypeptides/AARSs here. The term ‘fragment” here refers to the fact that the FF contains only a portion of a whole/parent PPT or NS; e.g., FFs typically are not made by fractionation. E.g., in aspects, CEP(s) include gDS FF(s)/gDVS(s), which typically exhibit one, some, most, or all (OSMOA) of the functions of WT counterpart(s) (WTC(s)). In aspects, a gDS FF or glycoprotein D (gD) variant sequence (gDVS) in an EP exhibits suitable, comparable, or improved function(s) with respect to a WT counterpart. In aspects, a gDS FF or gDVS exhibits less than all functions of a corresponding WT AARS. Functions of gDS(s) include (1) glycoprotein D signal sequence (gDSS) functions; (2) receptor binding (e.g., nectin-1, nectin-2, or HVEM) (in gDRBD(s)) and target cell-binding (e.g., DCs, T cells, epithelial cells, or fibroblasts); (3) promoting uptake of the associated gDP (e.g., in a gDFP, such as a gD Ag fusion protein (gDAgFP)); (4) enhancing ER processing of the gDP; (5) enhancing GPI anchoring (6) membrane association (in a gD transmembrane domain (TMD)); or (7) any other measurable function associated with gDD(s) or combinations of any such functions. As discussed elsewhere, gDP(s) in Eps can include non-functional WT gD AARSs or FVs thereof, but typically such AARS(s) do not characterize the gDP unless explicitly stated. gDP(s) also can comprise gDD(s) that are not discussed in detail herein but that exhibit function(s) (e.g., gDP(s) can be characterized by, i.a., inclusion of gDS(s) that DOS contribute or cause oligomerization/dimerization of gDP(s)).

In aspects, CEPs are characterized in, i.a., (1) comprising a combination of Ag(s), gDP(s), or both, and (2) PPT(s)/AARS(s) that are (a) immune cell internal target modulator(s) (ICITM(s)) (e.g., immune cell signal transducing adaptor protein(s) (ICSTAP(s))); (b) checkpoint pathway (CP) signal transducing adaptor proteins (STAPs) (CPSTAP(s)); (c) CP signal transducing adaptor protein modulator(s) (STAPM(s)); (d) ITICITM(s) (e.g., ITICSTAP(s), e.g., EAT-2 PPT(s) or EAT-2 PPT(s)+SAP PPT(s)); or (e) innate trained immunity peptidic immunomodulators (ITIPIM(s)). In aspects, expression products (EP(s)) can be characterized within ≥2 of such categories of PPT(s) (e.g., an EAT-2 PPT is an ICITM, a CPSTAP, and an ITICITM). In aspects, EP(s) include innate trained immunity immunomodulators (ITIMs, aka ITPIMs). Terms such as “innate trained immunity” are still evolving in the art. Regardless of the final standard in the art for such terms, an “innate trained immune cell” (ITIC) herein means any immune system cell(s) identified presently with innate trained immune responses, including DCs, NKCs, monocytes, and the like (PCMs are provided in references cited in the Background). An “innate trained immunity immunomodulator” here means any EP that DOS modulates IR(s) in ITIC(s). In aspects, ITIM(s) induce DOS immunological memory IR(s) in such cell(s), Ag-specific responses in such cells, or both. In other aspects, ITIM(s) induce more general IR(s) in such cells, which are not associated with detectable immunological memory or Ag-specific IR(s). Other non-immune cells (fibroblasts, endothelial cells, etc.) also exhibit innate trained immunity or similar effects but such cells are not considered innate trained immunity cells (see, e.g., Shao et al. Arterioscler Thromb Vasc Biol. 2020 June; 40(6): e138-e152), even though ITIM(s) may also induce IR(s) in such non-IC(s). Nonetheless, induction of IR(s) also in such cell(s) by delivery of EA(s) of CEPESC(s) is another aspect of the invention.

In aspects, Ag(s) in CEP(s) comprise feature(s) associated with target sequences (TS(s)) (e.g., non-glycoprotein D (gD) (NGD) TS(s)), or both. In aspects, one, some, most, generally all, or all (OSMGAOA) Ag(s) in CEPs comprise FF(s) or FV(s) of EL WT Ag(s), but DOS induce IR(s). In aspects, OSMGAOA Ag(s) in CEPs comprise AgV(s). In aspects, OSMGAOA AgV(s) in CEPs comprise (de-immunization variation or variant) (DIV(s)), e.g., glycosylation site removal variant(s) (GSRV(s)). In aspects, OSMGAOA AgV(s) comprise editope(s). In aspects, AgV(s) comprise removal of decoy epitope(s)/Ag(s), immunodominance reduction variation(s) (IRV(s)), or combinations. In aspects, Ag(s) in CEPs comprise both MHCI epitope(s) (MHCIE(s)) and MHCII epitope(s) (MHCIIE(s)), the combination resulting in DOS CD4 TC, CD8 TC, and B-cell (BC) IR(s) in TR(s). In aspects, OSMGAOA Ag(s) in CEPs are in PE(s). In aspects, one, some, most, generally all or all Ag(s) are associated by flexible linkers (FL(s)), mid-sized linker(s) (MSL(s)), or mid-sized flexible linker(s) (MSFL(s)). In aspects, OSMGAOA Ag(s) are associated with NGD TS(s). In aspects, such TS(s) are ITS(s). In aspects, such ITS(s) are PTPS(s), e.g., polyUb(s). In aspects, FP(s) in CEPs comprise self-cleavage site(s) (SCS(s)). In aspects, OSMGAOA Ag(s) are expressed in FPs, e.g., gDAgFP(s). In aspects, OSMGAOA Ag(s) in CEP(s) are expressed apart from gDP(s). In aspects, OSMGAOA Ag(s) are encoded by NAM(s) other than NAM(s) encoding a gDP or NAM(s) encoding ICITM(s), CPSTAP(s), ITICSTAP(s), or ITIPIM(s). In aspects Ag(s) comprise a mix of Th1/Th2/Th17 T cell epitope(s) (TCE(s)) and other features (e.g., DIV(s)) that DOS reduce cytokine syndrome adverse events (CSAE(s)) in TR(s). In aspects, Ag(s) comprise clinically relevant antigen(s) (CRA(s)) or putative CRA(s) (PCRA(s)).

In aspects, NAM(s) of CEPESC(s) comprise EEI(s) associated with OSMGAOA EPES(s). In aspects, NAM(s) comprise(s) strong constitutive universal promoter(s) (SCUP(s)) AW OSMGAOA EPES(s). In aspects, CEPESC(s) comprise 2+ NAM(s) comprising different NS(s). In aspects, NAM(s) are NAV(s). In aspects, NAV(s) are mRNA NAV(s) or DNA NAV(s). In aspects, DNA NAV(s) are plasmids. In aspects, NAV(s) are AW TFA(s). In aspects, TFA(s) are CaPNP(s) that DOS enhance IR(s) in TR(s).

In aspects, CEPESCs are employed in methods including new conditions of use (e.g., conditions of use not described in the Wistar Art). In aspects, CEPESC(s) are used to treat/prevent disease-causing agent associated disease(s) (DCAAD(s)) in TR(s) treated with or at risk of treatment with a leaky vaccine. In aspects, CEPESC(s) are used to treat/prevent DCAAD(s) in non-DCA immunosuppressed target recipient(s) (NDISTR(s)). In aspects, CEPESC(s) are used to treat/prevent DCADCAAD(s) in TR(s) that are non-HVEM-expressing TR(s) (e.g., pigs). In aspects, CEPs comprise PCV Ag(s) and CEPESC(s) are used to treat/prevent other non-human animals (NHAs), humans, or CT. In aspects, CEPs comprise PRRSV Ag(s) and CEPESC(s) are used to treat/prevent PRRSV in pigs, other NHAs, or humans. In aspects, CEPs comprise ASFV Ag(s) and CEPESC(s) are used to treat/prevent ASFV in pigs, other NHAs, or humans. In aspects, Ag(s) comprise coronavirus (COV) Ag(s) and methods comprise treating/preventing COV in TR(s). In aspects, Ag(s) comprise influenza Ag(s) and methods comprise treating/preventing influenza. In aspects, Ag(s) comprise an alpha-herpes virus (a-HV) Ag(s) and methods comprise treatment/prevention of a-HV diseases, e.g., EHV.

In aspects, Ag(s) comprise cancer Ag(s) (e.g., neoantigen(s), tumor associated antigen(s), oncogene(s), and the like). In aspects, methods comprise treatment of bladder cancer, lymphoma, or other cancer(s) in TR(s) including companion animals, humans, or both.

One aspect provides, i.a., CEPESC(s) including EPES(s) encoding an EP comprising gDAgFP(s) and ICITM(s), e.g., ITICITM(s), e.g., ITICSTAP(s). In aspects, EPs comprise ITICSTAP(s) that comprise EAT-2 PPT(s)/AARS(s) or a combination of EAT-2 and SAP PPT(s)/AARS(s). In aspects, an EAT-2 PPT/AARS is a EL WT EAT-2 PPT (e.g., human EAT-2), an FF of a WT EAT-2, or a FV. In aspects, Ag(s) of any EP(s) described here are associated with ITS(s), such as PTPS(s), such as polyUb(s), e.g., SEQ ID NO:1.

Another aspect provides CEPESCs comprising an effective amount (an “EA”) of expression product encoding sequence nucleic acid molecule(s) (EPESNAM(s)) including EPES(s) encoding EP(s) including gDAgFP(s) & AgV(s). In aspects, one, some, most, or all (OSMOA) of the AgV(s) are contained in gDAgFP(s). In aspects, OSMOA of the AgV(s) are expressed as separate PPTs from the gDAgFP(s). In aspects, OSMOA AgV(s) of the EP are GSRAgV(s) and in aspects OSMOA of the GSRAgV(s) are NLGSRAgV(s). In aspects, other parts of the EP also (i.e., also or alternatively) comprise GSRV(s) (e.g., in the gDS or other possibly included aspects of the EP, such as, e.g., an ITII).

In aspects, NAM(s) include(s) EEI(s) & NASM(s). In cases CEPESCs comprise NAM(s) including (a) glycoprotein D (gD) antigen (Ag) fusion protein (FP) encoding sequence (ES) (gDAgFPES(s)) & (b) EEI(s) that DOS enhance gDAgFP expression (and in cases DOS enhance gDAgFP-associated IR(s)). In cases EPESNAM(s) also comprise SCUP(s), NASM(s), or CT. In aspects, expression product encoding sequence (EPES) NAM(s) (EPESNAM(s)) are NAV(s), e.g., mRNA NAV(s) or plasmid DNA NAV(s).

An additional aspect comprises CEPESCs including EPES(s) encoding EP(s) that comprise gDAgFP(s) comprising MgDS(s). In aspects, MgDS(s) exhibit DOS reduced affinity for HVEM (with respect to HSV gDs); comprise DIV(s), GSRV(s), or both; exhibit enhanced affinity for a non-HVEM receptor; lack a functional gD TMD; lack a profusion domain (PFD); or comprise CT. In aspects, a gDAgFP comprises Ag(s) positioned downstream of any gDS in the gDAgFP. In aspects, gDAgFP(s) comprise Ag(s) positioned between gDSs in gDAgFP(s). In aspects, a MgDS is a chimeric gDS, is MCRT to a WT gDP of a virus endogenous to NHAs, e.g., pigs, dogs, horses, or cows. In aspects, such CEPs include immune cell (IC) target immunomodulator(s) (ICTIM(s)), CPSTAP(s), ITIPIM(s), or ITICITM(s) (e.g., ITICSTAP(s), e.g., EAT-2 PPT(s)).

Another aspect provides CEPESC(s) including effective amount(s) (EA(s)) of nucleic acid vector(s) (NAV(s)) comprising nucleotide sequence(s) (NS(S)) including gD antigen (Ag) fusion protein (FP) encoding sequence(s) (ES(s)) gDAgFPES(s) & (b) being associated with TFA(s), such as CaPNP(s), that DOS IR(s) in TR(s). In aspects, CEP(s) thereof comprise Ag-associated TS(s), e.g., ITS(s); MHCIE(s) and MHCIIE(s); AgV(s) such as GSRAgV(s); MgDS(s); or CT. In aspects, NAM(s) comprise EEI(s), NASM(s), or CT.

In aspects, the invention provides CEPESC(s) comprising ≥2 NAM(s). E.g., in aspects a composition includes (1) a first NAM comprising gDAgFPES and (2) a second NAM comprising (a) an immunostimulatory nucleotide sequence (ISNS); (b) ES(s) encoding a 2nd type of gDAgFP, (c) ES(s) encoding ICITM(s), ICSTAP(s), CPSTAP(s), ITIPIM(s), ITICITM(s), or ITICSTAP(s) (e.g., EAT-2 PPT(s)); (c) ES(s) encoding non-gD checkpoint inhibitor(s) (NGDCI(s)), non-gD immune cell receptor (ICR) targeting sequence (NGDICRTS) fusion protein(s) (FP(s)) (NGDICRTSFP(s)), nucleic acid non-checkpoint innate peptide immunomodulators (NANCIPI(s)) (e.g., cytokine(s)), antigen fusion protein(s) (AgFP(s)) (e.g., a FP comprising AgV(s), a polyepitope (PE) FP (PEFP), or AgFP that is both), Ag(s), or other peptidic immunomodulator(s) (PIM(s)); or combinations. In aspects, NAM(s) comprise expression-enhancing intron(s) (EEI(s)), are nucleic acid vector(s) (NAV(s)) (e.g., TFA-associated NAV(s)), or both. In aspects, EP PPTs comprise Ag-associated ITS(s) (e.g., PTPS(s)), DIV(s), GSRV(s), or combinations. In aspects, such CEP(s) comprise non-checkpoint immunomodulator polypeptide(s) CIMP(s), e.g., cytokine(s) (e.g., Th1 cytokine(s), e.g., IFNg or IL-2) or peptidic adjuvant(s) (PAj(s)) (e.g., heat shock protein(s)).

Another aspect is a CEPESC comprising an EA of NAM(s) encoding a gDFPAg or other gDP and ≥1 non-gD checkpoint inhibitor (NGDCI). In aspects, the NGDCI is a PD-1/PD-L1 checkpoint inhibitor (CI) or a CD112R CI. In aspects, NGDCI(s) comprise a multimeric extracellular targeting sequence (ETS) PPT lacking antibody sequences (e.g., a PD-L1 or CDR112R trap protein). In aspects, gDS(s) of the CEPESC also act as CIs in TR(s). In aspects, the CEP also comprises AgV(s), Ag-associated ITS(s), MgDS(s) (e.g., non-HVEM binding gDP(s)), ICITM(s), CPSTAP(s), ITIPIM(s), ITICITM(s), ITICSTAP(s) (e.g., EAT-2 PPT(s)) or combinations. In aspects OSMOA of the NAM(s) are NAV(s), e.g., TFA-associated NAV(s), e.g., CaPNP-associated plasmid(s).

In aspects, a CEPESC comprises an EA of NAM(s) comprising gDPES(s) and NGDICRTSFPES(s) (i.e., non-gD immune cell receptor (ICR) target sequence (TS) fusion protein (FP) encoding sequence(s)). In aspects, NGDICRTSFP(s) comprise ITIC ICR TS(s). In aspects, innate trained immune cell (ITIC) immune cell receptor (ICR) targeting sequence(s) (TS(s)) (ITICICRTS(s)) comprise DEC-205-TS(s). In aspects, an innate trained immune cell (ITIC) immune cell receptor (ICR) targeting sequence (TS) fusion protein (FP) (ITICICRTSFP) is a multimeric PPT and, in aspects, ITICICRTS(s) lack antibody (Ab) sequences (e.g., in cases ITICICRTSs are DC receptor (DCR) targeting sequences (TSs) (DCRTSs), e.g., DEC-205-binding keratin AARSs or variants). In aspects, the CEP comprises an ICITM, ICSTAP, CPSTAP, ITIPIM, ITICITM, or ITICSTAP, such as an EAT-2 PPT or AARS. In aspects, such a CEPESC is delivered to a TR that has pre-activated DCs (or the CEPESC comprises or is AAW activated DC(s)). In aspects, the NGDICRTSFP comprises Ag(s), such as AgV(s), and optionally ITS(s), such as PTPS(s), such as polyUB(s). A related aspect is a method of inducing IR(s) in a TR comprising delivering an EA of a CEPESC encoding a gDAgFP having any features described herein, and optionally (i) comprising ICITM(s), ICSTAP(s), CPSTAP(s), ITIPIM(s), ITICITM(s), or ITICSTAP(s) (e.g., EAT-2 PPT(s)) or (ii) being delivered AAW with delivering an EA of a NAM encoding ICITM(s), ICSTAP(s), CPSTAP(s), ITIPIM(s), ITICITM(s), or ITICSTAP(s) (e.g., EAT-2 PPT(s)), and in further AAW with delivering an effective amount (EA) of a composition comprising NAM(s) including non-gD immune cell receptor targeting sequence fusion protein encoding sequence(s) (NGDICRTSFPES(s)).

An aspect provides CEPESCs that are combination compositions (CCs) including ≥1 of any above described or otherwise provided CEPESCs and further comprising EA(s) of combination composition components (CCC(s)) that DOS enhances IR(s) associated with CEPESC(s) or induce IR(s) not induced by the CEPESC alone. In aspects, CCC(s) are vaccines. In aspects, CCC(s) are therapeutics. In aspects, CCC(s) are anti-pathogen or anti-cancer Abs. In aspects, CCC(s) are immune cells (IC(s)) (e.g., Ag-pulsed DCs) or other cells (e.g., bacterial vector(s)).

Aspects also include methods comprising delivery of EA(s) of any of the above-described or otherwise provided CEPESCs to induce IR(s) in TR(s). In aspects, method(s) are performed to induce prophylactic IR(s) in TR(s). In aspects, method(s) induce a therapeutic CE(s) in TR(s). In aspects, method(s) are repeated ≥2 times (using the same or different CEPESC(s)). In aspects, method(s) are AAW delivering EA(s) of CCC(s) or AAW CCEPM(s) or CCEPC(s).

Another aspect provides methods of inducing IR(s) in non-HVEM-expressing TR(s) comprising delivering an EA of CEPESC(s) to the non-HVEM TR one or more times. In aspects, the CEP comprises or predominantly comprises (PC), generally consists of, substantially consists of, or consists of (PCGCOSCOOCO) gDS(s) that exhibit DOS reduced HVEM binding in HVEM-expressing TRs. In aspects, the NS(s) encode a NASM. In aspects, the NAM(s) are TFA-associated NAV(s), such as CaPNP-associated DNA plasmids. In aspects, the CEP comprises non-gD (NGD) peptidic checkpoint inhibitor(s) (PCI(s)) (NGDPCI(s)). In aspects, the CEP comprises AgV(s), such as GSRAgV(s). In aspects, the TR is a pig and the CEP comprises Ags against PCV, PRRSV, or ASFV. In aspects, OSMOA of the Ags of the CEP are associated with ITS(s), such as polyUb(s). In aspects, the EPESNAMs comprise EEI(s). An aspect provides a method of inducing IR(s) against a leaky vaccine DCA, in TR(s) infected with a leaky vaccine DCA, or in TR(s) treated with a leaky vaccine effect vaccine comprising delivering to the TR(s) an EA of a CEPESC. In aspects, the leaky vaccine DCAAD is an influenza, EHV, or PCV.

Another aspect provides induction of IR(s) in TR(s) in NDISTR(s) comprising delivering EA(s) of a CEPESC to such TR(s) (e.g., a cancer patient). In aspects, the Ag(s) of a CEP PCGCOSCO or CO pathogen DCA-associated Ag(s). In aspects, such Ags comprise MHCIE(s) & MHCIIE(s), yet induce DOS BC, CD4 TC, & CD8 T cell (TC) IR(s). In aspects, method(s) result in DOS numbers of Ag-specific ICs in TR(s) not present before delivery of CEPESC(s) (e.g., TCs, natural killer cells (NKCs), or DCs)

A further aspect provides a method of inducing IR(s) comprising delivering EA(s) of CEPESC(s) ≥2 of the CEPESC(s) encoding different CEPs. In aspects, a 1st CEPESC comprises, e.g., a 1^{st} Ag, AgFP, or gDAgFPES & a 2nd CEPESC comprises an encoding sequence (ES) encoding (a) different gDAgFP(s); (b) NGDAgFP(s), e.g., an NGDICRTSAgFP; (c) Ag(s) (e.g., AgV(s), PE(s), or combinations); (d) ICITM(s), ICSTAP(s), CPSTAP(s), ITIPIM(s), ITICITM(s), or ITICSTAP(s) (e.g., EAT-2 PPT(s)); or (e) any combination.

### DETAILED DESCRIPTION OF THE INVENTION

The invention described here provides new methods for inducing IR(s) in TR(s) comprising delivering effective amounts (EA(s)) of compositions (complete expression product encoding sequence composition(s) (CEPESC(s)) that include antigen-encoding sequences (AgES(s)) in combination with additional factor(s) that enhance immune responses (IR(s)) resulting from the expression of such AgES(s) in subjects (target recipients (TR(s)).

In aspects, CEPESC(s) comprise gD protein (gDP) encoding sequence(s) (ES(s)) (i.e., gDPES(s)) and AgES(s). In aspects, gDPES(s) and AgES(s) are contained in 1, 2, or ≥2 nucleic acid molecule(s) (NAM(s)). In other aspects, CEPESC(s) are free of gDPES(s). In such aspects, CEPESC(s) can comprise, i.a., other immune cell targeting sequence(s), which can, in aspects, form part of non-gD fusion protein(s) (FP(s)) with other AARS(s).

Some aspects are characterized by one, some, most, or all (OSMOA) gDP(s) in a complete expression product (CEP) expressed from constructs being gD:Ag fusion protein(s) (gDAgFP(s)).

CEPESCs (sometimes referred to as compositions) also can comprise additional immunostimulatory feature(s), e.g., innate trained immunity peptidic immunomodulator(s) (ITIPIM(s)) or an internal target immunomodulator (ITI), e.g., immunomodulatory signal transducing activator protein (STAP) (e.g., an ITICSTAP, such as an EAT-2 PPT or an EAT-2 PPT and SAP PPT). Ag(s) of CEP(s) can be associated with ITS(s), such as PTPS(s), e.g., polyUb(s). Ag(s) also can be associated with de-immunization variant(s) (DIV(s)), such as glycosylation site removal variant(s) (GSRV(s)). CEPESCs also can comprise modified gD protein(s) (MgDP(s)). CEPESCs also can include non-gD PCIM(s) (NGDPCIM(s)) (e.g., PD-L1 or CD112R trap proteins). CEPESCs also can comprise non-viral vector NAVs with one or more unique properties (e.g., growth in a triclosan selection system). CEPESC(s) often comprise ≥2 NAVs comprising different NSs and often different ESs. NAVs of CEPESCs are often associated with TFAs, such as CaPNPs. EPES NAMs typically comprise EEI(s). The combination of some, most, generally all, or all (SMGAOA) of such elements provides CEPESCs advantageous properties lacking in compositions of the prior art, including gDFPES constructs of the Wistar Art. Methods of the invention provide new uses for such compositions that further enhance the applicability of such compositions in new ways, such as in the treatment of diseases in non-HVEM TR(s) or in non-immunosuppressed TR(s).

Typically, CEPESCs are designed for use in ≥1 alphaherpesvirus infectable vertebrate target recipients (TRs). TR(s) can be any suitable type of such subjects. In aspects, TR(s) is/are associated with unique features, such as not expressing HVEM, having been previously treated with a “leaky vaccine,” not having a checkpoint inhibition immunosuppressed state, or a combination thereof (CT).

In one aspect, the invention provides composition(s) wherein such composition(s) comprise only sequence(s) encoding naturally occurring amino acid sequences or wherein the antigen-encoding sequence(s) of such a composition mostly, generally only, substantially only, or only encode naturally occurring/native sequences (sequences of proteins expressed typically in typical pathogen(s) or DCA(s) (e.g., cancer(s) that are targeted by the composition). In aspects, composition(s) do not comprise one or more, e.g., two or more, three or more, or, e.g., collections of four or more, or any number of, non-native/non-natural T-cell epitope(s) (e.g., epitopes that are mutants or synthetic epitopes). In aspects, the invention provides composition(s) which do not comprise sequences resulting from, e.g., do not comprise sequences generated by a process or method comprising, epitope prediction. In aspects, the invention provides composition(s) comprising (e.g., mostly, generally, or only comprising) sequence(s) encoding antigen(s) that comprise, mostly comprise, substantially consist of, or consist only of partial or complete contiguous sequences of native protein(s) in a target or related pathogen/DCA. Such construct(s) can comprise non-natural amino acid sequence(s)/fragments outside of the epitope(s)/antigen sequence(s) expressed by the construct, but in association therewith in a fusion protein. E.g., in aspects, construct(s) encode one or more native antigen(s) that are linked by artificial/non-natural linker(s) (e.g., flexible linker(s)), as discussed elsewhere herein.

In aspects, compositions or methods herein are further characterized by lacking one or more elements of any of the references cited herein, including any of the elements disclosed in, e.g., US 2019/0351040 (Moderna).

A detailed discussion of the various components of CEPESCs, EPs/CEPs, and related methods of use, etc., follows, focusing on each component in turn. Uncontradicted, each type of element can be combined with any other suitable element(s) as exemplified by stated aspects of the invention.

### I. CEPESCS

CEPESCs can comprise NAM(s) and methods can comprise the administration of NAM(s) to TR(s). NAM(s) can comprise ≥1 discrete nucleotide sequences (NSs) including EPES(s) and possibly other NS(s), e.g., EEIs. Terms such as “nucleic acid molecule,” “nucleotide sequence,” “nucleic acid construct,” “nucleotide” or “polynucleotide” are related (and often can be used interchangeably). A “nucleic acid molecule” or “NAM” typically means a particular type (i.e., a particular copy) of a NAM, rather than a single molecule. NAM-containing compositions/CEPESCs typically comprise more than 100,000,000 and often more than 1,000,000,000 copies of any single referenced NAM. Typically, NAM(s) & other agents in compositions/methods are present/delivered/used in effective amounts (EA(s)). Nucleic acid molecules (NAM(s)) can comprise, primarily comprise, or consist essentially, generally consist, or substantially consist of ≥1, ≥2, ≥3, ≥4, or greater than 5 (e.g., 3-15, 4-12, 4-10, or 5-10) discrete NSs. “Discrete” with respect to NSs means having a distinctly identifiable structure and function that separates the sequence from other sequences of the NAM in which the NS is positioned (e.g., unique encoding sequences (ESs) or non-coding functions).

NSs typically are presented here in single strand form (regardless of whether the sequence is part of a single stranded or double stranded NAM), in the 5′ to 3′ direction, using traditional 1-letter nucleotide symbols. In passages, biomolecules, such as NAMs/NSs and other elements are labeled with labels such as “1^{st},” “2nd,” or “3rd,” etc. Unless indicated, the use of terms such as “1st NS” and “2^{nd} NS” is only for clarity and is not meant to indicate a specific order/arrangement of such sequences. In contexts, NSs described herein are described as “functional” nucleotide sequences (FNSs). Most NS elements are “functional,” meaning the NS exhibits DOS intended functions/properties in intended cells/target recipient(s) (TR(s)). Functions include expressibility, enhancement of expression, and induction/enhancement of IR(s), e.g., from immunostimulatory NS(s) (ISNS(s)).

In aspects, CEPESC(s) comprise only 1 NAM (e.g., a single type of plasmid or 1 type of mRNA). In other aspects, CEPESCs comprise ≥2 NAMs (e.g., 3, 4, 5 or more nucleic acid molecules, such as 2-20, 2-12, 2-10, 2-7, 2-6, 2-5, 2-4, or 2-3 NAMs). In aspects, NAM(s) comprise(s), primarily comprise(s), or generally, essentially, substantially, or only consist of RNA(s). In aspects, NAMs comprise, GCO, or CO DNA(s). In aspects, NAM(s) is/are single stranded. In aspects, NAM(s) is/are double stranded. Each NAM typically will be stable under typical expected conditions of use/storage (e.g., ≥6, ≥9, ≥12, ≥18, ≥24 months at RT (about 15-25/20° C., ambient (e.g., −50° C. to 50° C., −40° C. to 40° C., −35° C. to 35° C.), or accelerated stability temperature testing condition and humidity (per US FDA guidelines). CEPESCs typically are stable (per regulatory requirements) and non-toxic (do not induce DOS toxicity in TR(s)). In aspects, NAM(s) is/are relatively non-immunogenic, not resulting in significant unintended IR(s) in ≥1 TR(s) (e.g., as determined by clinical trial(s)). However, in other aspects NAM(s) in compositions are inherently immunogenic (e.g., NAM(s) comprising ISNSs). NAM(s)/CEPESCs are typically sterile. NAMs typically exhibit transient expression of ESs therein and do not DOS integrate into TR genome(s) (i.e., are not persistent gene therapy).

In aspects, one, some, most, generally all, or all NS(s) in a composition are constructs. The term “construct” refers to NS(s) comprising 2 smaller and discrete NSs, which typically are heterologous and exhibit different and measurable function(s) (e.g., a nucleotide sequence (NS) comprising a Cytomegalovirus (CMV) EEI, ≥2 Ag ESs, and intervening sequences encoding either a self-cleavage site (SCS) from another virus (e.g., a T2A SCS from asigna virus 2A), an artificial linker-encoding sequence, or both).

Uncontradicted, references to virus species generally, such as CMV, provide support for species of such viruses that infect humans and viruses recognized in the art under the same classification that infect NHA(s). Thus, e.g., the term CMV implicitly provides support for HCMV as well as other CMVs.

One type of construct that is typically combined with other NS(s)/construct(s) to form still larger constructs (e.g., vectors, described elsewhere) is an “expression cassette.” Many aspects relate to NAM(s) comprising at ≥1 expression cassette (e.g., ≥1 expression cassette comprising ≥1 gDPES). In aspects, CEPESCs & methods comprise NAMs comprising ≥2 expression cassettes (e.g., 2-4 or 2-3 expression cassettes). An “expression cassette” means a construct comprising (a) ≥1 coding sequence (CS) (sometimes called “a gene”) & (b) ≥1 regulatory sequences/elements. Typical expression cassettes comprise a promoter operably linked to ES(s).

A “coding sequence” (CS) (aka, an EPES) means a NS that is expressible in at least target host cells under suitable conditions. Expression products (both AARSs & nucleic acids) are sometimes referred to as “cistrons.” A CS can be a WT CS or can be an EPES having a synthetic CS but encoding a WT PPT, due to redundancy/degeneracy of the genetic code, splicing, etc. The term “open reading frame” (ORF) is also used to refer to CSs, but the term ORF typically is limited to a single expression sequence. In aspects, NS(s) comprise a “gene,” which can comprise 1, 2, 3, or more ORFs. The term “subsequence” is used sometimes to refer to a NS encoding part of an ORF, such as part of a fusion protein (FP). However, subsequences are typically just referred to as “sequences” herein. Readers will be able to determine based on context whether a sequence is a subsequence or an entire sequence.

NSs can have any suitable length. Most CSs will be at least ˜15 , ˜20 , ˜25, or ˜30 nucleotides (nt) (bases) in length. Some NSs are significantly larger. E.g., gDSES/gDPES can be, e.g., ˜100-2100, 200-1800, 300-1500 bases, 450-1250, or ˜600-1200 nt in length (per coding strand). In aspects, CEPESCs comprise NAMs that comprise at least about 2 kilobase pairs, ≥2.5 kbp, or ≥2.75 kbp, e.g., ˜2.5-4 kbp/kb, ˜2.5-5 kbp, or ˜3-5 kbp). The abbreviation “bp” means “base pair(s)” and “nt” means “nucleotides.” A kilobase pair (kb, or kbp) is 10³ bps & a megabase pair (Mb/Mbp) is 10⁶ bps.

In aspects, NAM(s) is/are in linear form (e.g., a mRNA or a linear expression element (LEE)) or a circular form (e.g., a DNA plasmid). NAMs can be double-stranded or single-stranded forms. A NS can be a DNA or RNA NAMs can be in single, double, triple, or quadruple stranded form (see, e.g., U.S. Ser. No. 10/407,695 & US20030100113). Nucleotide sequences (NSs) can have any suitable origin(s) and content(s). E.g., NSs can comprise prokaryotic sequences, eukaryotic mRNA, cDNA from eukaryotic mRNA, genomic DNA sequences from eukaryotic DNA, synthetic DNA sequences, and CT. E.g., NSs can comprise ≥1 sequences from e.g., a virus genome (e.g., a non-infectious viral genome), messenger RNA (mRNA), complementary DNA (cDNA), plasmid DNA (pDNA), or derivatives of pDNA. RNA or DNA NAMs can include, e.g., cDNA, genomic DNA, or synthetic DNA. NSs can include a gene or gene fragment and can comprise exons, introns, or both. NSs can be recombinant nucleotides, synthetic nucleotides, or comprise both. In aspects, NAMs are DNA molecules. DNA molecules comprising sequences encoding immunogenic proteins or that are otherwise immunogenic can be referred to as “DNA vaccines.” However, the use of the term “DNA vaccine” is not meant to necessarily limit the application of such a NAM to prophylactic applications. In aspects, NAMs in CEPESCs PC, GCO, or consist of (CO) DNA NAM(s).

In aspects, NSs/NAMs are composed of RNA, such as a messenger RNA (mRNA). RNA may be, in cases, in the form of a tRNA (transfer RNA), snRNA (small nuclear RNA), rRNA (ribosomal RNA), mRNA (messenger RNA), or micro-RNA (miRNA). “RNA vaccines” in aspects DOS induce both humoral & cellular immunity. In aspects, RNA vaccine constructs DOS outperform corresponding DNA-based vaccines in terms of inducing IRs. RNA can be produced in vitro, i.e., outside cells, using a DNA template containing a coding sequence, such as an AgES, or can be generated synthetically. In aspects, an RNA vaccine NAM of a CEPESC is a non-replicating mRNA. In aspects, CEPESCs comprise RNA vaccine NAM(s) that are self-replicating mRNA that comprises/requires a 2^{nd} RNA to ensure copying of the RNA vaccine NAM in vivo (e.g., an alphavirus-derived system, such as an alpha virus, picornavirus, or flavivirus system). In aspects, an RNA vaccine NAM is encompassed within a cell or other composition for delivery to a subject, such as a mRNA vaccine-loaded DC. In aspects, RNA NAM(s) comprise a capping modification, a polyA sequence (e.g., a polyA of about 100-200 nt, such as about 120-150 nt), a 5′-UTR (e.g., human heat shock protein 70 5′-UTR), one or more pseudouridine nucleosides, a phosphorothioate backbone, or a combination of any or all thereof, which DOS increase expression, half-life, etc. RNA vaccine technology is described in US20200325182 and references cited therein.

In aspects, CEPESCs comprising RNA sequence(s) encoding gDP(s) and Ag(s), such as gDAgFP(s) are provided. In aspects, RNA(s) are self-replicating RNA molecule(s). In aspects, RNA sequences comprise(s) a cap/capping modification (e.g., a 7-methyl-guanosine residue joined to the 5′-end via a 5′-5′ triphosphate; SFE Banerjee A K, Microbiol Rev. 1980 June; 44(2):175-205), a stability/expression promoting polyA sequence (e.g., a polyA of about 100-200 nt, e.g., ˜120-150 nt), or CT. In aspects, an RNA molecule (RNA) further comprises one or more of (a) a 5′-UTR (e.g., human heat shock protein 70 5′-UTR), (b) one or more pseudouridine nucleosides, (c) an at least partial phosphorothioate backbone, or (d) a combination of any or all thereof, which detectably increases expression, half-life, or both of the RNA. In aspects, RNA(s) are loaded into and delivered in a dendritic cell. In aspects, an RNA is delivered with a NAM encoding an ITII, such as an EAT-2 polypeptide-encoding DNA NAM. In aspects, an RNA molecule also comprises at least one PTPS-encoding sequence. In another aspect, NSs of an RNA molecule also encode(s) at least one epitope, at least one deglycosylation antigen variant, or both. In aspects, RNA molecule FNSs also encode at least one linker AARS positioned between at least two Ags, a cleavage site (e.g., a 2A sequence or an intein), or both. In aspects, an RNA comprises sequences encoding at least one MHC I antigen against a disease-causing agent (DCA) and at least one MHC II antigen against the same or a related DCA. In another aspect, an RNA also encodes B cell antigen(s). In aspects, RNA(s) lack(s) any sequences encoding a B cell antigen. In cases, RNA(s) comprise FNSs encoding an MHC I antigen and an MHC II antigen, either or both optionally comprising flanking sequences, without any B cell antigen to the disease-causing agent, wherein the expression of the RNA results in a detectable, typically enhanced, often significantly improved, and in cases both significant and clinically relevant cytotoxic T lymphocyte/CD8 (CTL) IR, a T-helper/CD4 (TH) IR, and a B cell IR. In aspects, RNA(s) include(s) NSs encoding cancer antigen(s). In aspects, RNA(s) comprise(s) NS(s) encoding pathogen antigen(s) (e.g., viral antigen(s)). The invention also provides methods of treating/preventing diseases comprising delivering to an alphaherpesvirus infectable vertebrate host (TR) an effective amount (EA) of any such composition (comprising such RNA(s)) or two or more of such compositions (e.g., in a prime/boost administration regime). In certain aspects, methods are performed in TR(s) that do not regularly express HVEM, e.g., swine. In cases, encoded antigens may be PCV antigens, PRRSV antigens, or ASFV antigens. In aspects, compositions and method comprising both RNA and DNA NAMs having characteristics as described in connection with NAMs discussed herein are provided (e.g., the invention provides a method in which a DNA vaccine construct is delivered followed by an RNA vaccine construct, or vice versa, or a combination of such constructs are delivered). In general, any of the various aspects and facets of the invention described herein can, where suitable, be performed with RNA constructs, as exemplified by the general use of “nucleic acid molecule” in most aspects and facets of this disclosure.

“Nucleotides” (aka, NTs/nts or bases) are monomeric units of NAMs. NTs comprise bases and [compound] backbones. NAMs can be artificial nucleic acids that contain other types of backbones from WT NSs, but still retain ordinary bases. E.g., NSs can comprise ≥1 artificial or modified nucleotides. However, in aspects most, generally all, or all (MGAOA) NSs comprise only conventional/natural sugars, bases, & linkages. In aspects, NSs include both conventional bases and substitutions (e.g., bases linked via a methoxy backbone or including ≥1 base analogs). In aspects, NAMs/NSs include nucleic acids containing analogs of natural NTs that have similar binding and other functional properties, and that typically are metabolized in a manner similar to naturally occurring NTs. Examples of modified nucleotides (e.g., 2′ methoxy substitutions (see WO 98/02582), inosine or other alternative bases (see, e.g., The Biochemistry of the Nucleic Acids 5-36, Adams et al., ed., 11^{th} ed., 1992), derivatives of purine or pyrimidine bases (see WO 93/13121), oxetane modified bases, xenonucleic acids (e.g., Pinheiro, V. B. et al. Science 336, 341-344 (2012)), or “abasic” molecules. Additional examples of modified nucleotides are listed in, e.g., the USPTO MPEP § 2422 (9th Revision—2018).

NSs can include conventional phosphodiester bonds or non-conventional bonds (e.g., an amide bond (e.g., WO 95/32305)), a methylphosphonate backbone, or another alternative backbone. In cases, NSs comprise backbones modified by replacement of ≥1 phosphodiester bonds with phosphorothioate bond(s). E.g., Neilsen P E, Curr Opin Struct Biol 9:353-57 & Raz N K et al, Biochem Biophys Res Commun. 297:1075-84. In aspects, phosphorothioate(s) in the backbone DOS improve cellular uptake, bioavailability, or both (PMCs in Eckstein F. Nucleic Acid Ther. 2014; 24(6):374-387). In cases NSs comprise chirally controlled oligonucleotides (SFE US 20200080083 & US 20020137921). In aspects, NS(s) comprise a modification that promotes DOS prolonged stability in one or more environments, e.g., NSs with chiral phosphorus linkages such as phosphorothioate or boranophosphate intermonomer linkages (SFE U.S. Ser. No. 10/457,945, U.S. Pat. No. 9,278,990 and WO 2018141908A1). In cases NSs comprise branched nucleotide sequence(s) (SFE Daring J, Hurek T. RNA. 2019; 25(1):105-120).

In aspects, NAMs/NSs include features that promote pre-expression processing, e.g., NAMs that that comprise introns, such as self-splicing introns (SFE U.S. Pat. No. 6,010,884). NSs can comprise sequences that result in splice modifications at the RNA level or at a DNA level by way of trans-splicing mechanisms prior to transcription (see, e.g., Chabot, Trends Genet (1996) 12(11):472-78; Cooper (1997) Am J Hum Genet 61(2):259-66; & Hertel et al. (1997) Curr Qpin Cell Biol 9(3):350-57).

Expression cassette constructs typically include 1+, 2+, or more regulatory elements. “Regulatory elements” include any non-coding sequence(s) that aid in expression of CS(s), the stability of NS(s)/NAM(s), or both. Regulatory elements (aka, regulatory sequences) include NSs that promote, enhance, or control expression/transcription of other NSs (CSs).

Typical regular elements include, e.g., a promoter, an enhancer, an initiation codon, a stop codon, and a polyadenylation (polyA) signal/sequence. Regulatory elements are typically operably linked to CS(s). Regulatory elements can include enhancer-promoter combinations, or other NSs that affect the expression or transcription of an associated, typically downstream NS (e.g., an expression-enhancing intron). Regulatory elements can be of various origins, both natural and synthetic, as exemplified throughout this disclosure. In aspects, DNA(s) comprise, PC, generally consist of (GCO), or only contain multi-cell-operable promoter(s) (aka ubiquitous promoters), able to effectuate promoter functions in several different cells/TRs, typically without significant loss in functioning. Examples of ubiquitous promoters include the CMV IE promoter, the EF-1α promoter, PGK promoters, UCB promoters, Ubiquitous Chromatin Opening Element (UCOE) promoters, and the GUSB (hGBp) promoter (SFE Husain et al. Gene Ther. 2009; 16(7):927-932).

In aspects, promoters of NSs PC, GCO, or CO one or more tissue/cell specific promoters, exhibit promoter activity only in certain cell types/tissues or exhibit significantly enhanced expression in certain cells/tissues. Examples of tissue specific promoters (e.g., the α-myosin heavy chain promoter) are described in US20200325182 and its cited references. In other aspects, construct(s) of are free of any tissue-specific promoters. In cases promoters are associated with enhancer elements (e.g., a CMV enhancer), or other promoter elements (to form a hybrid promoter), to DOS enhance expression. Relevant PMCs are described in, e.g., Ara Hacobian et al. Tissue Engineering Part B: Reviews. June 2018.226-239.

Promoters in constructs can be constitutive promoters, inducible promoters, or repressible promoters, etc. In aspects, promoters in constructs comprise, PC, GCO, or CO constitutive promoters (generally, unregulated promoters that generally continuously enhance transcription of associated coding sequences, under most or all physiological conditions of target cells). Constitutive promoters include the CMV IE promoter, EF1α promoter, PGK promoters, SV40 promoters, UBC promoters, human beta actin promoter, U6 promoters, the H1 promoter, chicken β-actin (CBA) promoter (and the CAG hybrid/derivative promoter), the R glucuronidase (GUSB) promoter, and ubiquitin C (UBC) promoters. Alternatively, promoter(s) comprise, PC, GCO, or CO inducible promoter(s), which are up- and or down-regulated in response to an appropriate signal/factor (aka an inducer). Inducible promoters adaptable to aspects include tetracycline response element (TRE) promoters or similar promoters in inducible expression system (see, e.g., Weber W et al. Methods Mol Biol. 2004; 267:451-466.), etc.

In aspects, promoters used in constructs include, primarily comprise, generally consist of, or consist of one or more viral promoters. In aspects, promoter(s) comprise, PC, generally consist of, or consist of one or more non-viral promoters. Non-viral promoters include the EF1α promoter and the human beta actin promoter, among others, as well as hybrid promoters (e.g., a CAG promoter) and synthetic promoters.

In aspects, promoter(s) in constructs are “strong promoters.” E.g., the CMV IE promoter (particularly when matched with the CMV IE enhancer), the EF-1α promoter, CAG promoter (when matched with the CMV IE enhancer), the SV40 early promoter, and TRE promoters are considered strong promoters, whereas the UBC (human ubiquitin C promoter) and GUSB, e.g., are not considered to be strong promoters. Methods & standards for the evaluation of strong and weak promoters are provided in, e.g., Powell S K et al. Discov Med. 2015; 19(102):49-57 and Qin J Y, et al. PLoS One. 2010; 5 (5): e10611. In aspects, ≥1 promoter(s) in constructs have a rate of inactivation that is the substantially similar or less than the CMV promoter. Promoters that have a lower rate of deactivation than CMV include the human elongation factor-1 alpha promoter (EF1α promoter/EF-1α promoter) and CAG promoter; whereas PGK promoters have a rate of inactivation that is similar to or greater than CMV promoters. In aspects, constructs comprise, PC, are generally associated with, or only contain promoters that are both strong promoters and promoters with low deactivation rate(s), such as a CAG promoter or an EF1α promoter.

In aspects, construct(s) comprise promoter(s) that is/are constitutive promoter(s), ubiquitous promoter(s), or constitutive and ubiquitous promoter(s). In aspects, promoter(s) is/are also strong promoter(s). In still another aspect, the promoter is also (i.e., also or alternatively) characterized as being a universal promoter. In aspects, OSMGAOA promoter(s) in constructs are “SCUPs” (strong, constitutive, & universal promoters).

In aspects, regulatory element(s), such as promoter(s), in constructs, result in DOS increase in expression of operably linked CS(s). In aspect(s), the regulatory element(s) result in “high level expression,” which means levels of expression at least 5-fold, 10-fold, at least 20-fold, at least 50-fold greater, at least 100-fold greater, at least 1000-fold (3-log) greater, or at least 10,000-fold (4-log) greater or more, within the first 1-, 2-, or 3-days following transformation than without the regulatory element(s), than typical levels of endogenous gene expression, or both. In another aspect, transformation with a construct of the invention results in “persistent” high level expression. Uncontradicted, the terms “transformation” and “transfection” are used the same here and are interchangeable with each other and “transduction,” wherever delivery of constructs through viral vectors is considered appropriate. “Persistent high levels of expression” means high level expression of the coding sequence(s) (EPES(s)/transgene(s)) (high meaning SCUP levels) persists for ≥1 week, typically ˜2 weeks or more, for example, ≥˜4 weeks, ≥˜7 weeks, e.g., 9 weeks or more, ≥˜12 weeks, ≥˜3 months, ≥˜6 mos, or ≥12 months. In aspects, expression level of transgene(s)/EPES(s) do/does not decrease more than 100-fold, e.g., no ≥50-fold, in some instances, not ≥10-fold in any such period following transformation from baseline levels observed within 1-, 2-, or 3-days post-transformation.

In aspects, promoters of constructs comprise, PC, GCO or CO promoters or promoter/enhancer combinations that have a size (or combined size) of less than 1.25 kbs (e.g., a UBC promoter), or less than 1 kbs (e.g., an SV40 early promoter, a PGK promoter). In aspects, promoters or promoter/enhancer combinations comprise, predominantly comprise (PC), generally consist of (GCO), or consist of (CO) (PCGCOOCO) promoters that have a size of ≥0.6 kbs, ≥0.8 kbs, ≥1 kbps, ≥˜1.1 kbs, or ≥1.5 kbs.

In aspects, promoter(s) comprise bi-directional promoter(s) (described in, e.g., U.S. Pat. No. 5,017,478) linked to ≥2 coding sequences (CSs).

NSs can also comprise other regulatory elements, e.g., ≥1 internal ribosome entry sites (IRESs), or RNA sequence enhancers (Kozak consensus sequence analogs), e.g., a tobacco mosaic virus omega prime sequence. Constructs can also comprise enhancer(s). In aspects, enhancer(s) DOS modulate expression specificity in terms of time, location, and expression level of operably linked CSs. Numerous highly effective enhancers are well known in the art (SFE Scharf D. et al. (1994) Results Probl Cell Differ 20:125-62: and Bittner et al. (1987) Methods in Enzvmol 153:516-544 for discussion). Suitable enhancers include, for example, RTE enhancers described in U.S. Pat. No. 6,225,082, and the human actin, human myosin, human hemoglobin, & human muscle creatine enhancers. The CMV enhancer, as noted above, is often paired with promoters, such as the CAG promoter. The WT HCMV enhancer is upstream of the HCMV promoter at −598 to −68 (˜600 bps). In aspects, enhancer(s), promoter(s), or a combination enhance expression of operably linked CDs 4-, 8-, 45-, 50-, and 90-fold. As is the case with the CMV promoter, the CMV enhancer is considered a “universal” enhancer, working in many cell types. Inclusion of such enhancers in constructs is an aspect. Both constructs comprising & lacking enhancer elements are aspects. In aspects, constructs comprise Ubiquitous Chromatin Opening Element (UCOE), which exhibit promoter/enhancer function(s). In aspects, UCOE sequences DOS prevent transgene silencing and promote consistent/high-level gene expression.

Regulatory element(s) incorporated into constructs, such as plasmids, can also include element(s) that act as transcription-termination element(s), a transcript-stabilizing element, a translation promoting element, or a nuclear export element, or a combination. E.g., DNA constructs typically include a polyA sequence that facilitates cleavage and polyadenylation of expressed transcripts. Suitable polyA sequences include the polyadenylation sequences of the bGH (Bovine Growth Hormone) gene, hGH gene, polyoma virus, TK (Thymidine Kinase), EBV (Epstein Barr Virus), rabbit beta globin, and the like. An exemplary polyA sequence usable in constructs is SEQ ID NO:10.

In aspects, constructs incorporate polyadenylation (polyA) sequence(s) that detectably or significantly increase nuclear export, expression, translation, expressed mRNA stability, or a combination of any or all thereof. In aspects, a polyA affords an at least about 125%, ≥˜150%, ≥˜ 200%, ≥˜250%, ≥˜275%, e.g., about 125-350%, ˜150-350%, or about 200-350% increase in expression as compared to a minimal synthetic polyA (SPA) signal in one or more target cells (SFE Levitt N et al. Genes Dev. 1989; 3(7):1019-1025 and Yew N S, et al. Hum Gene Ther. 1997; 8(5):575-584). Such polyadenylation (polyA) sequences include the SV40 (human Sarcoma Virus-40) polyA and the bGH polyA (bGHpA) sequence (see US20200325182 and cited references therein). In aspects, construct(s) comprise or only contain ≥1 polyA sequences that exhibit a strong polyA sequence effect (e.g., a polyA having enhancement effects similar to the SV40 late polyA or bGH polyA). A construct can have any suitable number of polyadenylation sites. Typically, each CS is associated with a single polyA site, but the expression of pre-mRNAs with multiple polyadenylation sites are possible (as in the case with VEGF-A mRNA). In aspects, constructs comprise polyA modulating cis-acting regulatory elements, e.g., polyA USE sequence(s) (see US20200325182 and cited references). In aspects, a polyA USE increases associated CS expression, by ≥125%, 150%, 175%, or ≥200%. In aspects, constructs comprise 1, 2, or ≥2 USEs & polyA(s) (e.g., SV40 2×USE, the HIV-1 USE, adenovirus (L3) USE, or the like).

In aspects, constructs incorporate a non-polyA post-transcriptional regulatory element sequence (e.g., a sequence that acts as an alternative transcript-stabilizing and/or nuclear export element), such as a woodchuck hepatitis virus post-transcriptional regulatory element (WPRE) or a constitutive transport element (CTE) of the simian retrovirus type 1 (SRV-1), or mouse RNA transport element (RTE), or a related hybrid element. In aspects, constructs incorporating such elements are associated with ≥1.5×, at least about 2×, ≥2.5×, ≥3×, ≥4×, or ≥5×increase in CS expression (e.g., ˜2-10×or ˜2.5-7.5×expression). See US20200325182 and cited references therein. Other regulatory sequences include transcription termination sequences, internal ribosome entry sites (IRES), splicing control sequences, and upstream activator sequences (UASs) (SFE U.S. Pat. No. 6,133,028 & U.S. Pat. No. 6,204,060). NAMs can include other specific initiation signals that aid in efficient translation of a coding sequence, such as a WT or FV Kozak consensus sequence (as described in U.S. Pat. No. 6,107,477 (e.g., GCCACCATG, GCCACCATG, or GCCGCCACCATGG (SEQ ID NO:458)). NAMs can comprise site-specific recombination sites, which can be used to modulate transcription of NSs (SFE U.S. Pat. Nos. 4,959,317, 5,801,030 & 6063627; EP 0987326; & WO 97/09439). NAMs can contain an origin of replication that confers the ability to replicate in the desired host cells, sites to enable incorporation of other NSs (e.g., a multiple cloning site (MCS), aka a polylinker (a short NS which contains many (e.g., 2-20, 3-15, 4-12, or 4-20) restriction sites, MGAOA being unique to the construct/vector). Constructs can also include nuclear targeting sequences (NTSs) that DOS enhance NAM delivery to the nucleus (e.g., part of the smooth muscle γ-actin promoter or the SV40-DTS sequence). In aspects, constructs can include any of the foregoing elements. In other aspects, constructs lack any one or more of such elements.

CS(s) of a construct and associated regulatory sequences will typically be operably linked (aka, operably associated). NSs are “operably linked” when they are associated in a manner as to DOS enhance/induce expression or carry out other intended function(s) (e.g., enhancement of mRNA transcript stability) when the CS(s) and regulatory sequence(s) are in proper location, orientation, etc., in the NS. E.g., a promoter is operably associated with CS(s) if the promoter DOS affects transcription of the CS(s). In cases, most, generally all, or all regulatory sequences in a NAM are operable in and the expressible sequences expressible in ≥2 or ≥3 species (e.g., in man). In aspects, sequences are expressible in is an even toed ungulate, e.g., a swine. NAMs can include NSs outside expression cassette(s), e.g., backbone elements.

In aspects, constructs comprise intron(s) (SFE Chapman et al., Nuc. Acids Res. (1991) 19:3979-3986). In cases, an element classified as another type of regulatory element also can comprise ≥1 introns. E.g., both EF1α and CAG promoters can comprise intron(s). In aspects, most/all other regulatory elements are free of introns. In aspects, introns can be associated with DOS improved mRNA processing, increased transgene expression, or both. In aspects, the constructs incorporate one or more introns that are associated with such other elements. In aspects, constructs incorporate one or more introns that are independent (separate) of any other regulatory elements. In aspects, constructs comprise expression enhancing intron(s) (EEI(s)), discussed below.

In aspects constructs comprise ≥1 EEI(s). An “expression-enhancing intron” (EEI) is an intron associated with DOS (e.g., several-fold) increase in mRNA processing, transgene expression, etc. In aspects, EEI(s) increase mRNA expression, levels of translatable mRNAs in the cytoplasm, or both. EEI(s) also can provide a DOS or several-fold increased rate of nuclear export to the cytosol, transcript stability, transcription initiation, transcription re-initiation, transcript elongation, transcript accuracy, polyadenylation, etc.

Phrases such as “several-fold increased,” “several-fold improved,” and “several-fold enhanced” here typically refer to a condition, event, effect, outcome, etc., in which the measured result (here, EEI-associated CS/EPES expression) is increased at least two fold (200%/2×), e.g., ≥3 fold, ≥four-fold, ≥five-fold, ≥six-fold, ≥seven-fold, ≥eight-fold, or at least ten-fold, at least 15-fold, ≥20-fold, or even ≥25-fold. In aspects, EEI(s) of a construct increases expression of the associated coding sequence(s) (e.g., on average) by ˜125%-˜750%, e.g., ˜150%˜600%, ˜175%˜600%, or ˜200%˜550%.

In aspects, constructs comprise EEI(s) that are part of a larger regulatory element, such as a promoter, enhancer, or combined promoter/enhancer. E.g., constructs comprising a CAG promoter, that includes a CMV IE enhancer sequence; the promoter, first exon, and first intron of the chicken beta-actin gene, and the splice acceptor of the rabbit beta-globin gene.

In aspects, constructs comprise EEI(s) that are independent of any other regulatory elements (e.g., are not part of a promoter or enhancer), at least as such elements are defined in respect of the construct (such an exclusion does not exclude placement of the intron in a 5′ or 3′ UTR). E.g., constructs provided herein can comprise a CMV Intron A sequence, such as SEQ ID NO:8 or SEQ ID NO:9 or a FV that is very related (VR), highly related (HR), or substantially identical (SI) (VRHROSI) to either thereof, which may be included in a construct with a CMV enhancer, e.g., SEQ ID NO: 7 or a similarly related variant thereof, a CMV promoter, e.g., SEQ ID NO:6, an FF, or a FV thereof, or both, neither of which comprises a CMV Intron A sequence.

In cases constructs include ≥1 “intronic cassettes.” Intronic cassettes are described in US20200325182 and references cited there in can include, e.g., gggccgggcctgggccgggtccgggccggg (SEQ ID NO:7)), a pyrimidine-rich “tract,” e.g., a tract of 5-20 or 8-15 pyrimidines, a consensus branch point sequence (BPS), etc. In cases construct(s) include ≥EEI(s) that also enhance expression even in the absence of DOS splicing. In aspects, construct(s) also comprise EEI(s) that detectably associate with U1 snRNA.

In aspects, constructs include at least a portion, such as at least a 5′ portion, of one or more introns that are first introns in their native (i.e., endogenous, naturally occurring) sequences. CMV Intron A is an example of such an intron. First introns or FFs thereof, such as 5′ fragments thereof, often can exhibit intron-mediated expression enhancement over other introns, at least with a typically DOS higher frequency of success.

In aspects, intronic cassette(s) is/are operably linked to the same promoter that mediates expression of CS(s). E.g., intronic cassette(s) may be located upstream, or 5′, of the CS, i.e., between the promoter and the initiation codon for the CS. Also, intronic cassette(s) may be located within a transgene, i.e., flanked by two exons of a CS. Also, an intronic cassette may be located downstream of the transgene (e.g., upstream of a polyA). An intronic cassette also can be separated from a transgene/CS by an IRES. In instances, an intronic cassette is under the transcriptional control of a promoter that is different than the promoter that mediates expression of the CS/transgene.

Examples of EEIs that can be incorporated/used in constructs include the EF-1α first intron, the MVM intron, and the human factor IX intron and variants thereof, and synthetic/artificial EEIs, such as a hybrid intron (e.g., an adenovirus SD intron/immunoglobulin heavy chain SA intron, the β-globin SD/immunoglobin heavy chain SA intron, βG1-IgG intron, & the SV40 late SD/SA intron. Examples are provided in US20200325182 and references cited therein.

In aspects, EEI(s) comprise CMV intron(s). In aspects, EEI(s) comprise HCMV Intron A, an FF, or a FV (e.g., a highly related, closely related, or substantially identical variant thereof) (a “CMV Intron A sequence”). E.g., a CMV Intron A sequence can comprise a deletion variant, wherein ˜10⁻⁶⁰⁰ nt of the WT Intron A sequence are removed/deleted (absent). In aspects, a CMV Intron A variant exhibits increased expression-enhancement as compared to EL WT CMV Intron A (see, e.g., Quilici L S et al. Biotechnol Lett. 2013; 35(1):21-27). In aspects, constructs comprising only a single intron are provided. In cases, only one CMV intron is included in a construct. In one example, the only intron sequence in the construct is a CMV Intron A sequence (or “Intron A sequence”). In aspects, at least a portion of the CMV IE first exon is also included in a construct. In aspects, the CMV IE first exon or portion thereof is the only CMV IE exon in the construct. In aspects, such a construct comprises a CMV promoter, CMV enhancer, or both, as well as an Intron A sequence. In aspects, constructs comprise a promoter that is at least partially heterologous to the CMV Intron A sequence, such as an RSV promoter or a CAG promoter. Examples of such constructs are described in U.S. Pat. No. 6,893,840 & US20120142056 and EEIs are also described in, e.g., Buchman et al., Molec. Cell. Biol. (1988) 8:4395-4405; Chapman et al., Nuc. Acids Res. (1991) 19:3979-3986; & Simari, R. D et al. Mol Med 4, 700-706 (1998). Constructs can comprise a CMV promoter, described elsewhere, and ≥1 introns heterologous to CMV. E.g., constructs can comprise NS(s) in which Intron A of CMV is substituted with a heterologous intron. Examples of such constructs are described in, e.g., US20090130126. Constructs comprising a CMV Intron A sequence may include CMV IE exons, such as all or part of exon 1 or exon 2. Examples of such constructs comprising a CMV promoter and heterologous introns are described in Nott A et al. RNA. 2003; 9(5):607-617.

In aspects, ≥1 intron/EEI is located 5′ to the associated coding sequence. In aspects, ≥1 intron/EEI is also located 3′ to the end of the associated coding sequence. In aspects, at least one intron/EEI is contained with a CS. In aspects, at least one, most, generally all, substantially all, or all of the introns included in the construct are located within 1-200 nt, such as 1-100 nt, 1-75 nt, 1-60 nt, 10⁻⁶⁰ nt, 20-60 nt, 30-60 nt, 25-55 nt, 35-55 nt, or 5-55 nt of the coding sequence (sequence end to sequence end).

In cases constructs comprise ≥2 introns/EEIs. In aspects, constructs also comprise ≥1 intron positioned internal to a coding sequence. For example, in one aspect the invention provides a construct comprising a sequence according to the formula intron1—gD-encoding sequence 1 (gD1) (coding for an N-terminal fragment of gD or a variant thereof)—coding sequences for antigenic sequence(s)—gD encoding sequence 2 (gD2, a C-terminal gD fragment or variant thereof)—intron 2—gD3 (corresponding to a second gD1 sequence, which may be the same or different from the first gD sequence)—antigenic sequence(s) coding region 2.

Terms such as promoter and enhancer are used differently (and sometimes interchangeably) in the art. E.g., a sequence can be called a promoter by some, while others may characterize the sequence as both a promoter & enhancer or a promoter & intron. E.g., SEQ ID NO:5 comprises a sequence described as a CMV promoter, but this sequence has significant overlap with SEQ ID NO:6 and SEQ ID NO:7, identified in the art as comprising the CMV promoter and enhancer sequences, respectively. Similarly, GenBank Accession No. M60321 defines CMV Intron A as corresponding to SEQ ID NO:8, whereas in the exemplary plasmids provided in the Example CMV Intron A is identified as associated with SEQ ID NO:9, which is identical to SEQ ID NO:8 for 825 nt (of 956 nt). Uncontradicted, all known functional forms of the referenced regulatory element herein are to be considered implicitly included by reference, as well as FVs thereof that are related, very related, highly related, or substantially identical (RVRHROSI) to any such known forms, to any recited element. E.g., references to CMV Intron A herein include SEQ ID NO:9, SEQ ID NO:8, and FVs thereof that act as EEIs and are RVRHROSI to either SEQ ID NO:8, SEQ ID NO:9, or both. Still other authors characterize CMV as comprising a “promoter/enhancer region” comprising binding sites for several different transcription factors, a complex enhancer, four exons, and three introns, intron A being the largest thereof. SFE Chapman et al., Nuc. Acids Res. (1991) 19:3979-3986 and Akrigg et al., Virus Res. (1985) 2:107-121, for a description of the corresponding region in hCMV strain AD169. Still others described HCMV IE1 “minimal promoters,” which may be, e.g., defined as a fragment of the HCMV IE1 promoter region capable of functioning as a promoter (e.g., a fragment of the HCMV IE1 gene comprising nucleotides−116 to +1 (the HCMV IE1 transcription start site)). Thus, references to a CMV promoter or enhancer, e.g., can be construed as alternatively but simultaneously and implicitly encompassing all such forms of these elements.

Nucleotide sequence(s) (NS(s)) can comprise other types of non-coding nucleotide sequences besides regulatory elements. E.g., where a nucleotide sequence is a vector, the NS can include “backbone” sequences. Non-coding sequences also can include, e.g., restriction enzyme site(s). One type of noncoding sequence that can be incorporated into NAM(s) of CEPESC(s) is a noncoding immunostimulatory nucleotide sequence (ISNS). Examples of ISNS include sequences that exhibit a CpG or UpA patterns. Typically, ISNS(s) induce DOS IR(s) in TR(s). In aspects, CEPESCs comprise CpG sequence(s) recognized by IC(s), e.g., innate IC(s) or ITIC(s), e.g., by binding an ICR or an innate trained immune cell receptor (ITICR). In aspects, a CEPESCs include NAM(s) comprising CpG NS(s) that binds a DC receptor (DCR). In aspects, the dendritic cell receptor (DCR) is DEC-205. In aspects, NAM(s) comprise class B CpG ODN. In aspects, CpG sequence(s) in NAM(s) DOS enhances uptake of the NAM by ITICs, e.g., DCs. In aspects, NAM(s) is/are optionally associated with a transfection-facilitating agent (TFA), such as a calcium phosphate (CaPNP) nanoparticle, which also can DOS enhance uptake of the nucleic acid by DCs.

CEPESCs also can include UpA-rich nucleotide sequences that can DOS induce IR(s), reduce NAM stability, or both. Such sequences are described in, e.g., Fros J J et al. Elife. 2017; 6: e29112. Additional types of immunostimulatory NSs that can be incorporated into CEPESCs are KNOWN. Examples include AU-rich sequences, CA- and CT-repeat sequences, (GTA)CATCC(CTA) motif sequences, U(C/G)U or U(U/A)N and (A/U)CG(A/U) motifs, U-rich RNA sequences, GAAAGACC motifs, and (C/T)(A/T)TTGT(T/C)ATG CAAAT motifs. PMCs are provided in US20200325182 and included references.

In aspects, NS(s) comprise other sequence(s) that DOS impact expression/EP(s) but are not ordinarily be considered regulatory elements. Examples of such sequences include splicing-related sequences, internal ribosome entry sites (IRESs—SFE Gritsenko A A et al. PLoS Comput Biol. 2017; 13 (9): e1005734, etc. One such sequence is a scaffold/matrix attachment region (S/MAR) sequence, which promote retention of vectors in daughter cells (as an episome) (e.g., after ≥10, 30, 50, 75, or ≥90 days or more post transfection, in ≥˜25%, ≥33%, ≥50%, ≥2× (+100%), ≥3×, ≥5×, ≥˜10×, or ≥˜20×cells). Other noncoding sequences that can be included in constructs include nuclear localization signals, ribosome binding sites, insulators, etc.

NAMs can comprise NSs that at least in part exhibit no function other than creating space between other NSs. Such “spacer” sequences can comprise elements that exhibit regulatory sequence functions, exhibit other functions, or that are nonfunctional (besides serving to separate NSs). Examples of such sequences include, e.g., 3′ and 5′ untranslated regions (UTRs). In aspects, nonfunctional spacer(s) are present that are associated with effects such as enhanced expression, nucleic acid stability, etc. NAMs can comprise any suitable number of noncoding regions/spacers, such as UTRs or more intergenic spacers (IGSs) located between CSs/expression cassettes. Each spacer can be, e.g., about 10 ˜1000 nt, e.g., ˜20 ˜800 nt, e.g., ˜30-600 nt, ˜40 ˜400 nt, or about 50 ˜250 nt, etc. Additional examples of noncoding/spacer NSs include nucleic acid vector backbones and also “stuffer sequences,” which are noncoding sequences used to achieve a certain size of vector for efficient packing, such as in adenoviral vectors. Stuffer sequences can comprise, e.g., 1-10 kb of noncoding sequences, e.g., 2-10, 2-7, or 2-5 kb.

NAM(s) of CEPESC(s) comprise CS(s)/EPES(s). A “coding sequence” (aka a “coding region,” CDS, CS, or EPES) means a portion of a NAM (a sequence) comprising codons translated, directly (in the case of mRNA), or indirectly (in the case of DNA), into AARs. Most CSs result in production of PPTs, though DNAs can in some cases also produce RNA end products. Typically, terms such as “express” or “expression” typically refer to the process to produce an RNA or PPT, including transcription and translation. Another related term that often is similarly used in the art, but that sometimes differs in scope, is “open reading frame” (abbreviated “ORF”). Uncontradicted, the term ORF is the same as a CS. No part of the disclosure is intended to limit the scope of any of these terms. Untranslated stop codons can be considered part of a CS, but flanking sequences, e.g., promoters, are not part of the CS.

As stated above, coding sequences are sequences that encode a polypeptide. Terms such as “protein,” “peptide,” and “polypeptide” (PPT) herein typically mean any peptide-linked chain of at least 2 AAs (typically at least five, ≥6, ≥7, or ≥8 AAs), joined by peptide bonds. Thus, as used herein, the terms refer to both short polymers (chains) of AARs, which are also commonly referred to in the art as peptides, oligopeptides, & oligomers, and longer AARSs, i.e., polypeptides or proteins. Proteins can include one chain or multiple associated chains. Thus, in a general sense “polypeptides” discussed herein can include, for example, homodimers, or heterodimers. A subunit of a polypeptide can be considered a “sequence,” “region,” or “domain,” of a PPT. In contexts, the term “domain” is used to identify distinct part of a PPT having one or more unique functional or structural properties, such as a defined tertiary structure. However, uncontradicted the term “domain” generally here is construed the same as “amino acid sequence” (aka, a “sequence” where context is clear that the sequence is an AARS). Specifically defined domains, however, (e.g., RBDs) can be defined/characterized by specific structure(s)/function(s).

PPT EPs may undergo processing in cells of expression (COEs). Uncontradicted, any PPT/AARS comprises all forms of the AARS/PPT with respect to possible posttranslational modifications (e.g., acetylation, phosphorylation, glycosylation, sulfatation, amidation, proteolysis, sumoylation, prenylation, etc.). In aspects, post-translational site(s) are “removed” in FVs. A single CS may result in 2, 3, 4 or more separate PPTs due to processing. “Removal” in such respects means deletion by/due to sequence changes.

In aspects, ≥2 CSs, such as ≥3 CSs, are present in a single NAM, e.g., on a single plasmid. In aspects, ≥2 CSs are in different constructs in a single CEPESC, e.g., on separate (different) plasmids. Both types of CEPESCs are aspects, as will be clear from other portions of this disclosure.

Amino acids that make up polypeptides and amino acid sequences described herein can be referred to herein by either their commonly known three letter symbols (e.g., “Gly” for glycine) or by the one-letter symbols recommended by the IUPAC-IUB Biochemical Nomenclature Commission (e.g., “A” for alanine). Expressed PPTs can comprise or CCCs can comprise peptides that include modified (e.g., derivatized) or other unconventional AARs (see WO2018039438 and US20200325182 for PMCs). E.g., in cases PPTs described herein may be components of useful provided compositions or used in methods. In such aspects, derivatized amino acids, such as those described above, may be incorporated into such PPTs. However, generally AARSs herein will typically consist of, consist essentially of, substantially consist of, or generally consist of residues expressible in COEs/natural amino acid residues.

Typically, reference to a PPT or AARS does not mean actual individual molecules or sequences, but, rather, such terms refer to types of molecules/sequences, typically present in ≥10³, ≥10⁶, or more copies.

In aspects, ≥1, at least some, primarily most, generally all, substantially all, or all NSs of a CEPESC (a) encode PPT(s) that are “naturally occurring,” (b) are “naturally occurring” polynucleotide sequences, or both. Terms like “naturally occurring” applied to a sequence or biomolecule refer to a sequence or a molecule having a sequence/composition that occurs naturally E.g., a NS encoding a naturally occurring gD sequence means a nucleotide sequence that expresses a gD sequence that occurs in natural herpesviruses. The term “naturally occurring” also can be applied to fragments (FFs) of biomolecules found in nature. Terms such as “wild-type” or endogenous are similarly used here and, accordingly, should be interpreted similarly. Such terms typically refer to the composition of the referenced sequence, molecule, or fragment, rather than the actual origin thereof. E.g., a wild-type DNA can be generated by DNA synthesis, rather than isolation from an organism.

In general, WT PPTs and NSs can be substituted by functional fragments, functional variants (aka, FVs or variants), and homologs. Accordingly, any referenced biomolecule or sequence herein should be interpreted as also implicitly disclosing corresponding aspects in which any suitable homolog, functional fragment (FF), or variant (FV) thereof are incorporated or used in place of the referenced biomolecule or sequence.

Suitability of FFs, variants/FVs, or homologs is typically determined by composition/structure as a referenced biomolecule or sequence, maintenance of some or all of the functions of the referenced biomolecule or sequence, at an appropriate level, and usually both.

A “variant” (“functional variant” or FV) means a PPT, NS, NAM, or AARS which is substantially similar in structure and at least similar in one or more respects in terms of biological activity to a referenced sequence/biomolecule. PPT/AARS variants include PPTs and AARSs, respectively, that differ from a native form of a polypeptide or amino acid sequence (WTC) because of one or more (conservative or unconservative) deletions, insertions, or substitutions of amino acid residues. Because deletions can define variation, in aspects a variant can be an FF, and vice versa. However, not all variants are functional fragments (FFs). Typically, the degree of “identity” between constituents of sequences (nucleotides or amino acid residues) in an optimal alignment is the indicator or an indicator of sufficient relatedness. FVs typically are related, very related, highly related, or substantially identical (RVRHROSI) to WTC(s).

Alternatively, the scope of suitable variant PPT/AARS can be defined based on “similarity” in the composition of AARSs. Specifically, variants will typically have a sequence that also is “similar,” if not very similar, highly similar, or compositionally equivalent (SVSHSOCE). Thus, polypeptides and amino acid sequences specifically described in connection with aspects (e.g., human EAT-2), will, uncontradicted, be understood to simultaneously implicitly disclose FVs defined by identity (being RVRHROSI)) or similarity (being SVSHSOCE). E.g., description of a construct encoding human EAT-2 also implicitly discloses constructs encoding FVs of EAT-2 that are RVRHROSI to human EAT-2 and variants that also have AARSs that are SVSHSOCE to human EAT-2, and which, in either case, exhibit 1+ EAT-2 function(s) at a suitable, similar, or improved level in TR(s). Thus, references to specific PPTs here, e.g., CMV intron A, human EAT-2, HSV-1 gD, HSV-2 gD, and the like, also implicitly encompass FFS of such referenced PPTs and FVs of either thereof.

Readers will also understand that any reference to a PPT, AARS, NAM, or NS herein similarly also implicitly provides corresponding disclosure for incorporation or use of an FF thereof. Functional fragments (sometimes aka “fragments”) will typically exhibit 100% local identity to a referenced sequence, but not complete identity due to missing portion(s) of the WTC. FFs are large enough to provide suitable, comparable, or, in some cases, even improved functioning with respect to the EL WT referenced biomolecule/sequence. In aspects, FFs comprise ≥33%, ≥50%, ≥65%, 75%, ≥80%, ≥85%, or ≥90% of a EL WT biomolecule. However, in aspects, as in the case of Ag(s), an FF can comprise only a small portion of an WTC EP (e.g., ˜5%, ˜7.5%, or ˜10%).

Reference to a PPT, AARS, NAM, or NS also implicitly provides support for aspects also including any suitable homologs of the referenced sequence/molecule. Suitable homologs, however, often do not exhibit relatedness or similarity to referenced biomolecules/sequences.

The degree of identity between 2+ NSs and 2+ AARSs is indicative of relatedness between such sequences. However, changes in the nucleotide sequence of a nucleotide sequence variant may not alter an encoded AARS due to genetic code degeneracy. As such, at least with CSs, relatedness of NSs may not be required to define s a suitable substitution regarding WT NSs.

FFs and FVs can be generated by any suitable methods, such as, e.g., mutation (e.g., site directed mutagenesis, random mutagenesis, etc.), synthesis, DNA shuffling, rational protein design, and the like. E.g., any suitable combination of deletion, insertion, & substitution modifications can be used to arrive at a FV, provided the variant retains at least one intended/required activity at a suitable, comparable, or improved level. AARS insertions can include amino and/or carboxyl-terminal fusions of from 1+ AAs to PPTs of essentially unrestricted length, as well as intrasequence insertions of 1, 2, or 2+ AARs. Intrasequence insertions (i.e., insertions “within” the sequence) can range generally from, e.g., about 1 to 15 residues, about 1-10 AAs, 1-7 AAs, 1-5 AAs, etc. (e.g., per every about 200, 150 residues, or 100 residues). PMCs are further provided in US20200325182.

Readers will understand principles applicable to generation of FVs without application of undue experimentation. E.g., introduced Cys and Met can be susceptible to rapid oxidation and multiple cysteines located in a peptide are susceptible to forming undesired structure-changing disulfide linkages. Similarly, aspartic acid can undergo hydrolysis and cause peptide cleavage under acidic conditions when paired with glycine, proline or serine. A series of glutamine, isoleucine, leucine, phenylalanine, threonine, tyrosine or valine can cause R sheets. Substitutions expected to produce significant changes in a PPTs properties include those where 1) a hydrophilic residue, e.g., seryl or threonyl, is substituted for (or by) a hydrophobic residue, e.g., leucyl, isoleucyl, phenylalanyl, valyl, or alanyl; 2) a Cys or Pro is substituted for (or by) any other residue; 3) an AA having an electropositive side chain, e.g., lysyl, arginyl, or histidyl, is substituted for (or by) an electronegative AA, e.g., glutamyl or aspartyl; or 4) an AA having a bulky side chain, e.g., phenylalanine, is substituted for (or by) an AA that does not have a side chain, e.g., Gly.

In aspects, constructs comprise NS variants with enhanced properties/function(s), e.g., in terms of stability, expression, and the like. Thus, for example, in aspects ≥1 CSs, regulatory elements, or both, are optimized in terms of, e.g., resulting in detectably or significantly enhanced expression, typically significantly or clinically improving the functionality of the sequence/NAM (in the context of the present invention promoting, inducing, or enhancing an immune response). In aspects, such enhancement is achieved through modifying a secretion signal (discussed further below), using codons for the most abundant transfer RNA (tRNAs) (codon optimization, discussed below), reducing impeding secondary structures on the resulting mRNA, introducing one or more expression-enhancing introns (a feature of the invention discussed in detail below), etc. In aspects, such enhancement is further achieved through, e.g., increasing the number of genes encoding a polypeptide in a construct or composition (to two or more copies); increasing the transcription of the gene (such as by placing the gene under the control of a constitutive promoter, strong promoter, universal promoter, or a promoter that exhibits a combination of some or all of such features); or increasing the translation of the NS/gene through various methods described here or in US20200325182.

In aspects, NS(s) also include other functionality-enhancing variations, e.g., variations that bias GC content to DOS increase mRNA stability or reduce secondary structures; minimize tandem repeat codons or base runs that may impair gene construction or expression; customize transcriptional and translational control regions; insert or remove protein trafficking sequences, remove/add post translation modification sites in encoded protein (e.g. glycosylation sites); add, remove or shuffle protein domains; insert or delete restriction sites; modify ribosome binding sites or mRNA degradation sites, to adjust translational rates or promote proper folding of EPs; or to reduce or eliminate problem secondary structures within the polynucleotide. In aspects, the constructs comprise one or more codon optimized sequences that exhibit detectable improvements in one or more of such features. In aspects, constructs comprise codon optimized sequence(s) for TR(s) (e.g., dogs, pigs, or humans), wherein the codon optimized sequence DOS increases expression; DOS reduce(s) silencing or blocking of expression; or both.

Any codon optimized sequence can be optimized to degree(s), for example a sequence can be “fully optimized” or “minimally optimized.” In aspects, constructs comprise NSs having a different degree of codon optimization, either within a single construct, between constructs, between compositions, or between compositions and administrations. Alternating the amount of codon optimized sequences contained in a construct or composition or used in a method can result in optimized long-term results.

In aspects, NS(s) are subject to full optimization. When using the “full optimization” method, the term “about” is used to account for fractional percentages of codon frequencies for a given AA. In aspects, NS(s) are minimally optimized. In a “minimal optimization” approach, coding regions are only partially optimized. For example, the invention includes a nucleic acid fragment of a codon-optimized coding region encoding a polypeptide in which at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95% or 100% of the codon positions have been codon-optimized for a given species. That is, they contain a codon that is preferentially used in the genes of a desired species, e.g., a vertebrate species, e.g., humans, in place of a codon that is normally used in the native nucleic acid sequence. E.g., some, most, generally all, or all codons that are rarely found in the genes of TR(s) are changed to codons more commonly utilized in the TR(s). In aspects, compositions and methods of the invention include delivery of a construct containing a sequence that is at least partially codon optimized for ≥2 species. E.g., a construct can be partially optimized for humans & dogs.

A sequence that is at least about 70% identical to one or more referenced sequences is “related” to such sequence(s). A sequence is “very related” to other sequence(s) if it is ≥80% identical to the referenced sequence(s) or contains no more than 2 AAR differences from a referenced sequence of ≤˜10 residues. A sequence is “highly related” to a sequence or sequence if it exhibits ≥90% sequence identity to such reference sequence or sequences or contains no more than 1 amino acid residue differences from a referenced sequence under 10 AARs. A sequence is “substantially identical” if it shares ≥96% identity to referenced sequence(s).

References to sequence(s) or related sequences also provides implicit support for AARSs that are SVSHSOCE, or both SVSHSOCE & RVRHROSI to a referenced “related” sequence (e.g., a WTC). The term “similar” when used in connection with sequence(s) also provides implicit support for sequences that are SVSHSOCE to referenced AARS(s).

In describing FVs regarding WT AARSs/NSs disclosure may refer to AAs/NTs “corresponding” to AAs/NTs in the referenced WT sequence(s). A “corresponding” AA/NT can be located in a different position than the referenced AA/NT, can (if suitable based on context, plausibility, etc.) have a different composition than the referenced AA/NT, or both. E.g., an AA corresponding to WT AARS AA 100 can occur at position 85, 95, 105, or 110 in a FV and can be a different AA, such as a conservative substitution.

In the art, terms such as “percent homology” and “sequence similarity” are sometimes also used to indicate “identity.” In most aspects of this disclosure, however, “similarity” with respect to amino acid residues means the inclusion of compositionally similar amino acid residues, as is discussed below, and “homology” as described above herein relates to two or more sequences or biomolecules identified in the art as being homologs of one another (rather than as a particular degree of similarity). Methods of assessing identity and similarity are known in the art (e.g., via the LALIGN program, available through, e.g., ExPASy (which focuses on local alignment of sequences); the UNIRPOT ALIGN tool, & the NCBI COBALT Constraint-based Multiple Alignment Tool). See US20200325182 for more PMCs.

FVs can comprise conservative or non-conservative AA substitutions, deletions, or additions, etc., with respect to referenced sequence(s) (e.g., a WT EL PPT). Typically, most, generally all, or substantially all substitutions of a FV are conservative substitutions. Typically, the number of deletions or “gaps” between AARs in FVs with respect to WTCs is limited (e.g., to ≤5%, ≤3%, or under 2% of total AARs/AAs). Substitutions with similar AARs (conservative substitutions) often can lead to comparably functional FVs. In cases where most of the differences between a variant polypeptide or sequence and a referenced polypeptide or amino acid sequence are defined by conservative amino acid substitutions, the variant can be considered to have a “similar” sequence or composition. Thus, for example, even though a variant may have a percent identity that would not indicate the sequence is “related” to a referenced sequence, it may be sufficiently similar in composition to a referenced sequence to exhibit similar, if not enhanced, function(s).

Like identity, “similarity” can be determined by amino acid comparison of two or more polypeptides or sequences in an optimal alignment. Examples of such grouping and related principles are described in, e.g., US20160151478. A “percent similarity” can be determined between 2+ compared PPTs/AARSs using such tools or manual calculation using principles KNOWN or described elsewhere. As with assessing sequence identity, approaches can lead to variation in the amount of identity, but, highly similar sequences will tend to have similar similarity scores, regardless of the approach taken to assess similarity. Because of such variations, use of the approximator “about” in connection with most measures of similarity values is appropriate.

A “similar” amino acid sequence typically exhibits ≥˜75% amino acid similarity to a reference sequence in an optimal alignment. A “very similar” sequence is composed of ≥85% similar amino acid residues to referenced sequence(s). A “highly similar” sequence is composed of ≥92% similar residues to one or more reference amino acid sequences. A [substantially] “compositionally equivalent” or (SCE/CE) is composed of ≥98% amino acid residues that are similar (conserved) with respect to a referenced AARS.

Alignment tools such as those described above for determining identity also often have the capability of determining similarity between sequences. For example, the LALIGN program will compare two sequences on the basis of both residue identity and similarity in an optimal alignment. The BLAST 2 sequences tool will produce alignments with identity and “positives” scores, which can be used in place of similarity scores generated by LALIGN or an average of the two can be used where analysis with either tool is suitable. Scoring matrixes typically are used in such analyses (see US202003251820. Such tools and methods are exemplary.

The disclosure of any referenced sequence or polypeptide also implicitly provides support for variants that are SVSHSOCE to the referenced sequence or polypeptide. E.g., a reference to human EAT-2 in an aspect would implicitly provide support for the use of human EAT-2 variants that comprise a similar sequence to EAT-2, which would include, for example, its murine homolog (which exhibits 88.6% similarity according to LALIGN and 81% positives according to BLAST (analyzed by BLAST 2 SEQUENCES)).

In some cases, such similar variants may also exhibit a high enough level of identity to a referenced polypeptide or sequence to be considered related in terms of sequence identity, as well as similar in terms of sequence similarity. Thus, for example, such polypeptide and sequences that are implicitly disclosed should be considered to include polypeptides and sequences that are at least related in identity to the referenced sequence or polypeptide and also SVSHSOCE to the referenced biomolecule or sequence. For example, full length HSV-2 gD may implicitly substitute full length HSV-1 gD as it exhibits about 82% sequence identity and about 93% similarity according to LALIGN analysis using the above-described settings and 78% identity and 84% positives according to BLAST using the same settings. Thus, even under the more stringent of these two tests these homologs would both be considered “related” in terms of identity and at least “similar” (highly similar according to LALIGN and very similar if taking an average of LALIGN and BLAST data).

Other statistical tools in the art also can be used to characterize similarity between two or more sequences. Examples of such tools are bit scores and E-values, which are, for example, reported as outputs of analysis by BLAST tools. An E-value ≥10e-100 usually also indicates the compared sequences are identical, substantially identical, or substantially compositionally equivalent. At 10e-50<E-value <10e-100, the sequences also can be considered highly similar. At 10e-10<E-value <10e-50, the sequences also can be considered to be very similar. At 10e-6<E-value <10e-10 the sequences also can be considered similar sequences. E.g., murine and human EAT-2 exhibit an E-value of 1 e-62 when compared by BLAST (using BLAST 2 Sequences), indicating that based on such criteria these polypeptides are “highly similar.” Typically, use of E-values may be limited to sequences of more than 40 amino acid residues, more than 50 amino acid residues, more than 60 amino acid residues, or more than 100 amino acid residues.

The bit score, which is another output of BLAST analysis tools and other bioinformatics sequence analysis software programs, gives an indication of how good an alignment is between two sequences. A bit score of 175 or higher (e.g., obtained using default BLAST settings in tool(s) mentioned above) also can indicate that two sequences are highly similar (e.g., an alignment of golden monkey and human EAT-2 sequences has a bit score of 248 with 99% identity), a bit score of 125-175 also can indicate two sequences are at least very similar (e.g., an alignment of murine EAT-2 and human EAT-2 is associated with a BLAST bit score of 173 at 65% identity and 81% positives), and a bit score of 75-125 can at least indicate that two sequences are similar.

Other conservative substitutions, e.g., involving substitutions of entire regions having similar hydrophobicity characteristics, are well known, and can be considered within the broader context of determining amino acid sequence similarity. Thus, changes that can be considered “conservative” can further be classified, in part, by considering the hydropathic index of amino acids, as understood in the art. Kyte et al., J. Mol. Biol. 157:105-132 (1982). Related principles for some polypeptides that may comprise surface-exposed and non-exposed, interior residues are known in the art (SFE Moelbert S, Emberly E, Tang C. Correlation between sequence hydrophobicity and surface-exposure pattern of database proteins. Protein Sci. 2004; 13(3):752-762. doi:10.1110/ps.03431704). Tools for such analysis are also available. E.g., the ProtScale tool allows users to analyze a sequence based on hydrophobicity, bulkiness, and polarity, etc.

In aspects, conservation in terms of hydropathic/hydrophilic properties also is substantially retained in a variant polypeptide or sequence as compared to a referenced (parent) peptide of sequence (e.g., the weight class, hydropathic score, or both of the sequences are at least about 50%, ≥60%, ≥70%, ≥75%, ≥80%, ≥85%, ≥90%, ≥95%, or more (e.g., about 65-99%) retained). Methods for assessing the conservation of the hydropathic character of residues/sequences are known in the art and incorporated in available software packages, such as the GRAVY program (available at gravy-calculator.de/) and the GREASE program (formerly available through the SDSC Biology Workbench) (see also, e.g., Kyte and Doolittle et al., J. Mol. Biol. 157:105-132(1982); etc.

Compositional similarity in NSs can also be assessed through NAM/NS hybridization techniques. E.g., by observation of selective hybridization under “moderately stringent” or “stringent” conditions. Nucleic acid hybridization methods are known (SFE Sambrook et al., 2001, Molecular Cloning, A Laboratory Manual, Cold Spring Harbor Press, N.Y. and Ausubel et al., 1989, Current Protocols in Molecular Biology, Wiley Interscience).

In aspects, a reference to a nucleotide sequence herein will be understood to implicitly also encompass/disclose any NS(s) that hybridize(s) to the defined sequence under stringent hybridization conditions (exemplified by ˜0.015M sodium chloride, 0.0015M sodium citrate at ˜65-68° C. or ˜0.015M sodium chloride, ˜0.0015M sodium citrate, & ˜50% formamide at ˜42° C.).

In aspects, as noted elsewhere, a PPT can be substituted with a homolog. Homologs are biomolecules or sequences that occur in natural organisms (e.g., vertebrates, viruses, bacteria, etc.). A homolog of a reference polypeptide can naturally occur in the same species or different species/type of organism than the species/type of the reference molecule/sequence. Unlike FVs, homologs having low levels of sequence identity can often suitably substitute for each other (as can FFs or FVs of such homologs). E.g., the gD homologs of alphaherpesviruses share only 22±33% amino acid identity according to Connolly et al. (2001) but share some functions. Thus, with respect to gDSs, homologs implicitly disclosed as alternative aspects can exhibit relatively low levels of identity, provided that such sequences either are art-recognized homologs or suitable FFs/FVs of such homologs. A homolog of a sequence that is a functional fragment of a biomolecule is a corresponding sequence from a homolog to a reference sequence, such as a corresponding domain, e.g., the transmembrane domain of a gD polypeptide, the nectin-1 binding domain of gD homologs, and the like. Examples of relevant homologs include gD homologs), HVEM homologs, and EAT-2 homologs, etc. A “homolog” in this disclosure does not necessarily have to exhibit classifical “homology,” in terms of evolutionary origin, particularly since proof of homology is extremely difficult, if not impossible, in most cases. A “homolog” herein can include, e.g., orthologs, paralogs, an allelic variant, and other “cognates” or counterparts of a referenced biomolecule/sequence recognized as a homolog or having homologous function and typically overall similar structure (if not AARS). In addition to arising from genetic relatedness, exemplified above, homologs can arise from alternate mRNA splicing events, from alternative proteolytic cleavage, genetic polymorphism, etc. Generally, a homolog will exhibit suitable, comparable, or improved level of ≥1 function(s). Thus, some are not homologs (e.g., where such paralogs lack any common function).

Similar structure and function of polypeptides is typically used to determine homology in the art. Homologs can, however, exhibit relatively low levels of identity, and still be classified in the art as being homologs. See US20200325182 for relevant PMCs.

Like FVs, homolog substitutes for referenced NSs are not limited to CSs, but can include regulatory sequences, e.g., promoters or EEIs. In aspects, constructs having features described herein are replaced with a homolog of a referenced sequence(s).

As noted, substitution with homologs of named PPTs is implicitly provided where suitable. E.g., in aspects HSV-1 gD sequences described herein can be replaced with a corresponding gD sequence from HSV-2 gD, a variant of an HSV-1 gD sequence, or a gD sequence from a type of alphavirus that typically infects other hosts, such as a chicken (Gallus HV gD), a horse (e.g., EHV-1 gD), a cow (e.g., BHV-1 gD or BHV-5 gD), or a pig (e.g., PRV gD). E.g., described HSV-1 gD constructs that comprise a soluble form of gD lacking a TMD can be replaced with a corresponding soluble portion of a mature HSV-1 gD homolog (e.g., a soluble PRV gD or EHV-1 gD, such as in an aspect where a construct encoding AAs 26-340 of HSV-1 gD (SEQ ID NO:82) is replaced with the homologous sequence consisting essentially of AAs 31-357 of Gallid Alphaherpesvirus-2 (GenBank Accession No. AAA64967.1)).

In aspects, one or more sequences encoded by constructs are homologs of corresponding sequences endogenous to the intended host/subject species or to a virus that infects the intended host/species. For example, in aspects, methods comprise use of constructs that encode one or more HSV-1 gD sequences which are administered to dogs, pigs, or cats, as opposed to the gD sequences of viruses that normally infect such species.

Although the term “chimera” is sometimes used in the art to refer to any fusion protein, in TD terms such as “chimera” or “chimeric sequence” are used to refer to molecules and sequences, respectively, that are fusion proteins that comprise 2 or more sequences MCRT different WT sequences that are homologs of one another. Such sequences/molecules also are sometimes referred to as “hybrids” or “hybrid sequences.” In aspects, biomolecules of compositions comprise chimeras. E.g., aspects include constructs encoding gD PPTs that comprise chimeric sequence(s), e.g., formed from a HSV-1 gD sequence and an HSV-2 gD sequence (e.g., the HVEM binding domain of the HSV-1 gD sequence is replaced with corresponding residues from HSV-2 gD or the HVEM-binding domain of HSV-1 gD or a substantially identical or highly related variant is fused with sequences corresponding to most, generally all, substantially all, or all of the portion of HSV-2 gD that are located downstream of the portion of HSV-2 gD that corresponds to the HVEM-binding domain of the fusion protein). An example of such a chimera is a gDP comprising AAs ˜26 to 55 of HSV-1 gD and AAs ˜56-393 of HSV-2 gD or residues ˜56-340 of HSV-2 gD. Fusion proteins comprising sequences from, substantially identical to, or at least highly related to any two or more (e.g., 3, 4, or 5) of the naturally occurring gD polypeptides discussed herein or other homologs thereof can be included in such chimeric fusion proteins. Thus, for example, gD chimeras can be formed from the fusion of gD sequences from chicken and turkey alphaherpesviruses; dog and cat alphaherpesviruses; human and horse alphaherpesviruses; human, dog, and cat alphaherpesviruses; and the like.

In aspects, variants of a referenced polypeptide or amino acid sequence can further be characterized on the basis of having a similar predicted/confirmed structure, in part or all of a biomolecule. E.g., a homolog can have a similar structure to a referenced counterpart domain/PPT. Any FV or homolog described herein implicitly includes FVs and homologs that are structurally similar. E.g., disclosure of an HSV-1 gD sequence will be understood to implicitly disclose use of variants of such sequence that are related or similar in sequence composition and also structurally similar in respect(s) to HSV-1 gD.

While variants will typically comprise a similar structure to a referenced sequence and homologs will likewise often share a similar structure, this may be in only part of a sequence or molecule.

Structural determinations of polypeptides and amino acid sequences can be made by any suitable technique, including by empirical analysis, computer modeling based on sequence information, or both. Experimental methods for assessing structural similarity of polypeptides that can be applied to determine structural similarity of variants include nuclear magnetic resonance (NMR) techniques (including two dimensional and three dimensional NMR methods), microscopy techniques such as cryo-electron microscopy, Raman spectroscopy small angle x-ray scattering techniques, circular dichroism methods; Fourier Transform Infrared (FTIR) spectroscopy; or X-ray crystallography (e.g., scattering or diffraction methods). In aspects, structural similarity of AARSs is determined by RMS deviation between corresponding superimposed portions of two determined structures for a variant and parent/referent. In aspects, the coordinates of at least 25, ≥30, ≥40, ≥50, ≥70, ≥80, ≥100, or ≥120 corresponding atoms, and in some cases ≥150, ≥200, or ≥250 corresponding atoms are used to calculate the RMS deviation between compared AARSs. In embodiments, an RMS deviation between structures suitable FVs or homologs to a WTC PPT/AARS is 3.5 Angstroms (A) or less, about 3 A or less, or about 2.5 A or less. In other aspects, the RMS deviation between two or more similar sequences that can define a subset of suitable variants implicitly provided by a disclosure of any referenced sequence or polypeptide herein is about 2 A or less, about 1.5 A or less, about 1 A or less, 0.75 A or less, 0.5 A or less, 0.3 A or less, 0.2 A or less, or 0.1 A or less. Aspects regarding performance of RMS are provided in US20200325182. In aspects, a PDB model is generated for each of the sequences to be compared and such a PDB model is used to generate a TM-score using the TM score tool available at zhanglab.ccmb.med.umich.edu/TM-score/. One group of variants within the implicit disclosure of any reference provided herein exhibits a TM score of at least 0.5, e.g., ≥0.6, ≥0.7, ≥0.8, or ≥0.9. In aspects, assessment of structural similarity is performed by methods comprising or CO computational sequence-based structure prediction. Several tools provide for analysis of sequences on such a basis. See US20200325182. In aspects, a variant will have an instability index score that indicates the variant is a stable sequence when analyzed using this tool (default settings). In aspects, FVs also will have an estimated eukaryotic half-life of at least 10, at least 20, or at least 30 hours. Methods for assessing similarity of peptides in terms of hydropathic properties, weight conservation, and similar considerations are further described in e.g., WO 03/048185, WO 03/070747, and WO 03/027246. Structural analysis also can be performed by comparison against one or more domain databases (e.g., Structural Classification of Proteins (SCOP), Cambridge University); the database of protein families (Pfam, Welcome Trust Sanger Institute); the Universal Protein Resource (UniProt, maintained by and available through the UniProt Consortium); the Integrated resource for protein families (InterPro; maintained by and available through EMBL-EBI); the Class Architecture Topology Homologous superfamily (CATH, maintained by and available through Institute of Structural and Molecular Biology at the University College London); and the families of structurally similar proteins (FSSP, maintained by and available through EBI). Any algorithm deemed suitable by one of skill in the art may be used to select the linker, including but not limited by those used by SCOP, CATH and FSSP. Useful examples of tools/algorithms used for such assessments include Pymol (Delano Scientific LLC), Insightll & Quanta (both from Accelrys), MIDAS (Univ. of California, San Francisco), SwissPDB viewer (Swiss Institute of Bioinformatics), TOPOFIT (Northeastern University), CBSU LOOPP (Cornell University), & SuperPose (Univ. of Alberta).

In aspects, variants are defined as structurally similar on the basis of exhibiting similar domains with a sufficiently strong score (e.g., an e-value) based on a domain analysis tool. Examples of available domain analysis tools include, e.g., Prosite (prosite.expasy.org/scanprosite), SMART (smart.embl-heidelberg.de), NCBI's Conserved Domain Database (Conserved Domain Architecture Retrieval Tool), INTERPRO (ebi.ac.uk/interpro), ProDom (prodom.prabi.fr/prodom/current/html/home.php), CATH v3.4 on the world wide web at cathdb.info), Superfamily (supfam.cs.bris.ac.uk/SUPERFAMILY), PIRSF (pir.georgetown.edu), and functional searched by PANTHER on the world wide web at .pantherdb.org. The ExPASy bioinformatics portal provides a listing of numerous tools for the evaluation of structure, including various ways to employ PROSITE analysis of sequences (e.g., the PPSearch tool).

Domain pattern databases that can be used to analyze one or more sequences are available in the art. Examples of such tools include PROSITE, Pfam, SMART, InterPro, PRINTS, and the like. Examples of such analyses are provided further below (e.g., with respect to gDS variants).

Predicted structural similarity in a test sequence, such as a variant amino acid sequence, in one aspect means exhibiting one or more of the same domains as a reference amino acid sequence analyzed by the same tool. Typically the determination of domains will be determined by (a) InterPro Scan (ebi.ac.uk/interpro/search/sequence/) or PFAM (pfam.xfam.org/) and also (b) NCBI's CDD search tool, or (c) by both positive hits with (a) and (b), wherein the association of both sequences and the one or more domains is associated with an E-value that indicates that the relationship is significant, thereby reflecting the likely presence of the domain. An E-value ≥10e-100 will typically indicate near certainty of the relationship; 10e-30<E-value <10e-100 indicates the relationship is highly likely; 10e-10<E-value <10e-30 indicates that the relationship is very likely; and at 10e-6<E-value <10e-10 the relationship can be considered likely. For example, HSV-1 gD is identified by Interpro Scan and PFAM submission as possessing a domain according to PFAM family PF01537 (“Herpesvirus glycoprotein D/GG/GX domain”) with an E-value of 8e-39 (highly likely) and CDD analysis similarly identified the likely presence of the domain with an E-Value of 4.34e-22 (very likely), whereas HSV-2 gD is associated with this domain with an Interpro Scan/PFAM E-score of 1.2e-37 (highly likely) and a CDD E-value of 5.88e-44 (highly likely). Analysis with these tools can also employ bit scores to assess relationship. A bit score of ≥175 also can indicate that the identified/expected domain maps to the domain or domain family with near certainty, a bit score of 125-175 also can indicate relationship between a program-identified domain and domain family is at least very likely (e.g., HSV-1 gD exhibits a bit score of 132.6 and HSV-2 gD exhibits a bit score of 128.8 to PFAM family PF01537 according to PFAM analysis), and a bit score of 75-125 indicates likely relatedness.

FVs typically retain at least a suitable (or comparable or improved) degree of one or more functional activities exhibited by the referenced sequence or biomolecule. E.g., regarding gD sequence function(s) include binding HVEM, Nectin-1, or another gD receptor (e.g., Nectin-2). With respect to Ag(s) or ITII(s) such a function might be the ability to stimulate an immune response in a host. In the case of an expression-enhancing intron the retained ability will be the ability to detectably enhance expression of the associated coding sequence(s). In the case of a promoter, the function will be the ability to enhance expression of an operatively linked coding sequence. A “suitable” level of activity in such contexts means at least about 33% of one or more referenced activities is exhibited by the variant (or “is retained” by the variant) if the function is associated with a measurable property. A “similar” or “equivalent” level of activity typically means ≥75% of the referenced activity (and typically ≥85%, ≥90%, or 95-100% of the referenced activity). An “improved” level of activity can be any detectable level of improvement of activity with respect to the function-in-question as compared to the reference sequence, such as a wild-type counterpart of a variant (e.g., about 105% of the typical activity of the referenced biomolecule or sequence). In aspects, FVs are provided that have a DOS level of improved activity (a statistically significant improvement). In aspects, variants are provided that have a clinically significant level of improvement (a statistically significant improvement in CEs associated with the variant as compared to the reference AARS/PPT).

Antigenic sequences, for example, are associated with the primary function of promoting, inducing, or enhancing an antigen response in a subject. ITII sequences exhibit the ability to immunomodulate the activity of one or more innate trained immune cells. Expression-enhancing intron sequences are associated with enhancement of associated coding sequences. PTPS sequences are associated with detectably enhanced levels of proteasomal processing. Some elements can be associated with two or more functions in some cases. For example, a gD sequence or polypeptide can be associated with binding to one, two, or more receptors; with checkpoint inhibition functions; with uptake of associated antigens by target cells; and the like. Such functions can also be defined as higher level functions such as the ability to treat a disease or condition or to induce a protective IR against a DCA. Various exemplary tests for such functions (e.g., induction of an immune response, treatment of disease, and the like) are described elsewhere in this description and additional examples of such tests will be known to those of skill in the art.

It also can be the case that one (O), some (S), most (M), generally all (GA) (OSMGA), but not all, of the functions of a referenced WTC are exhibited/retained in a variant, substitute homolog, chimera, or the like. For example, a gD homolog might exhibit nectin-1 binding, but not HVEM binding, despite the presence of both functions in the referenced sequence. Where specific functions are required for a class of biomolecules or sequences according to the explicit description provided herein, however, suitable variants also will exhibit such functions at a suitable similar, or improved level, or the level specified by the disclosure, if applicable.

In aspects, the duration of the retained/exhibited function also can be classified as either suitable, similar, or improved vis-à-vis WTC(s).

In aspects, variant sequences are further characterized in not resulting in a detectable, significant, or clinically significant increase in an aspect of toxicity, mutagenicity, genotoxicity, carcinogenicity, teratogenicity, deleterious cross-reactivity, or significant clinical adverse events in subjects of a species or other defined population when the construct encoding the variant sequence(s) is administered in an intended/effective amount under planned administration conditions (dosages, dosage amount, etc.). Assessment methods are provided in US20200325182. Such characteristics can also be applied to any EP(s)/compositions hereof.

Constructs can encode a number of AARSs or PPTs or both as well as gDP(s) and Ag(s). E.g., in aspects, constructs include NSs encoding a reporter sequence/polypeptide, a selection sequence/polypeptide, or both. In aspects, a reporter sequence/polypeptide, a selection sequence/polypeptide, or both, are expressed from the same NAM as another CS, e.g., a gDP. In aspects, a selection sequence/reporter sequence is expressed as part of a fusion protein with other sequences. In aspects, a selection sequence/polypeptide, reporter sequence/polypeptide, or a combination thereof, is expressed from a NAM that is different from the nucleic acid comprising the gD polypeptide-encoding sequence (e.g., the gD-antigen fusion protein-encoding sequence) or is different from any nucleic acid comprising any other encoding sequences, but included in the same composition that is used to transfect target cells.

A “reporter sequence/polypeptide” (aka a marker) means any suitable sequence or polypeptide that facilitates detection of the expression product. The use of such sequences/polypeptides are known. In general, a reporter gene can be any gene that is not present in or expressed by the recipient organism or tissue and that encodes a polypeptide whose expression is manifested by some easily detectable property, e.g., enzymatic activity, fluorescence, radioactive NTs/AARSs, otherwise tagged/labeled biomolecules, and the like. A “selection sequence/polypeptide” is a sequence that allows the growth of cells transfected with a construct comprising the sequence to selectively grow. A selection marker can be any coding sequence that allows for selective retention of cells comprising a nucleic acid of interest, e.g., a plasmid, during culturing and propagation in the host cells. Examples of selectable markers include those genes useful in antibiotic resistance systems, e.g. ampicillin, kanamycin, and neomycin and antibiotic resistance systems, e.g. amp, kana, neo systems. Selection markers can include genes to provide a genetic marker for selection and/or evaluation of the cells, such as to assess in vivo survival or localization; genes to improve safety, e.g., by making the cell susceptible to negative selection in vivo.

Constructs can include one or more nonantibiotic resistance markers/selectors. In cases, such constructs lack any antibiotic resistance markers/selectors. In practical terms, such features can be important in, e.g., constructs for administration to NHAs, particularly livestock subjects, where the use of antibiotics is strictly regulated, discouraged, or prohibited. Examples of such selection systems include toxin/antitoxin systems. In aspects, the constructs comprise NS(s) encoding a selection sequence that allows selection by growth in the presence of a non-antibiotic biocide, such as, e.g., triclosan (CAS Registry No.: 3380-34-5). Selection for host cells carrying plasmids can be based on, e.g.,/i.a., host tolerance to this biocide induced by overexpression of the bacterial gene fabI gene (encoding Enoyl-[acyl-carrier-protein] reductase [NADH]) incorporated into the plasmid. Goh S, Good L. Plasmid selection in E. coli using an endogenous essential gene marker. BMC Biotechnol. 2008; 8:61. Vibrio cholera FabV, a functional homologue of E. coli FabI, also can be used as a suitable marker for selection and maintenance of both high and medium-copy number plasmid vectors in E. coli in a triclosan selection system. SFE Ali S A et al. PLoS One. 2015; 10 (6): e0129547. CEPESCs can comprise NSs including CS(s) and comprising a triclosan selection marker gene, such as FabI or FabV.

In many aspects, CEPs comprise FPs, such as gDAgFPs. In aspects CEPs can comprise 1, 2, 3, or ≥4 gDPs/gDSs and 1, 2, 3, 4, 5, 6, 8, 10 or more Ag sequences. In aspects, SMGAOA of such gDS(s) and Ag(s) are contained in gDAgFP(s). Other FPs also are included in certain aspects, such as in compositions or methods in which a targeting domain is incorporated into a 2nd fusion protein-coding sequence, e.g., an innate trained immunity cell targeting sequence (ITICTS), e.g., a dendritic cell targeting sequence, e.g., a DEC-205 binding sequence, e.g., a DEC-205-binding trap protein.

A “fusion protein” generally refers to any polypeptide that, in at least one single chain, includes at least two distinct, typically heterologous AARSs (which are sometimes variously described herein and in the art as parts, portions, sections, ends, partners, or domains of the FP. Typically, the different portions/domains of an FP each have at least one distinct function. E.g., in the case of a gD-antigen FP, the gD portion(s) of the fusion protein impart receptor targeting functions and the antigen sequences induce specific DCA-associated IR(s). The term “domain” is used in connection with FPs to refer to such portions, but the term domain in such contexts is not meant to imply/require secondary, tertiary, or quaternary structure.

In certain embodiments, a fusion protein expressed or produced by a host cell (e.g., a DC or a T cell) locates to the cell surface, where the fusion protein is anchored to the cell membrane with a portion of the fusion protein located extracellularly (e.g., containing a binding domain) and a portion of the fusion protein located intracellularly (e.g., containing a signaling domain). A fusion protein can be engineered to include an EL of a PPT or only an FF of a PPT. The joining of the two or more genes expressing a FP may be made in any order, though in certain cases the placement of certain domains is preferably in a specific order, as will be indicated in the description of certain aspects.

In addition to the specific FP component sequences described herein, additional heterologous AARSs can be included in FPs without impairing the functions of the other domains (e.g., detection/purification tags/handles).

AARSs that form part of a FP can be linked directly or indirectly. An “indirect link” means ≥2 referenced AARSs are joined through an intervening AAR or AARS. Such an intervening residue or sequence can be called a “linker” or a “spacer” (or “tether”). Terms such as “linked” and “fused” are used interchangeably here to refer to the joining of any 2 sequences/parts.

In aspects, a linker DOS promotes functioning of part(s) of an FP. E.g., linker(s) may be sufficiently large, sufficiently flexible, or both to allow the linked reference sequences to function more effectively than compared to corresponding no linker or smaller/more rigid linker FPs. A linker typically primarily comprises or generally consists of uncharged amino acid residues. In aspects, linker(s) DOS reduce immunogenicity of an FP.

An FP can include any suitable number of linkers and other operative sequences. E.g., an FP can include 1-8 or more Ag sequences, optionally 1-5 gD sequences, and possibly other sequences, such as one or more PTPSs, endoplasmic reticulum (ER) targeting/processing sequences (ERTPSs), or a combination thereof, wherein the FP comprises 1-15 linkers.

A linker typically will primarily comprise, GCO, consist essentially of, or consist of a sequence of two or more neutral polar or nonpolar amino acids. Linkers can be any suitable length. A linker can be, for example, 2 to about 100 amino acids in length, such as between 2-, 3-, and 4- and about 20-, 35- or−50 AAs in length, e.g., 3, 5, 10, 15, 20, 25, 30, 35, 40, 45, or 50 AAs in length (e.g., 2-28, 2-22, 2-18, 2-14, 2-12, 2-10, 2-8, 3-12, 3-15, 3-18, 3-21, 3-9, 4-8, 4-6, 4-12, 4-16, 4-20, 5-20, 5-15, 5-20, or 5-25 AAs in length). An exemplary, non-limiting linker is an AARS comprising at least 2 residues, ≥3 residues, ≥4 residues or ≥5 AAs and is flexible, hydrophilic, and has little or no detectable secondary structure of its own. When multiple linkers are used to interconnect portion of the molecule, the linkers may be the same or different (e.g., the same or different length and/or amino acid sequence). In aspects, a linker can be “cleavable,” for example, by auto-cleavage, or enzymatic or chemical cleavage. In other aspects, OSMGAOA of the linkers of a fusion protein do not contain cleavage sites.

In aspects, FP(s) comprise linker(s) comprising one or more Gly residues. In aspects, FP(s) comprises 1, 2, 3 or more Gly-rich linkers (at least primarily comprises of Gly residues) or the linkers of FP(s) primarily comprise, generally consist of, or consist of Gly-rich linkers. In aspects, one, two, primarily all, generally all, or all of the linkers of a fusion protein comprise at least 33% Gly residues. In aspects, OSMGAOA linkers of an FP are flexible linkers. “Flexible” linkers are materially, primarily, generally, substantially, or entirely composed of glycine and/or serine residues (examples are provided in US20200325182 and included references). E.g., FPs can comprise linkers comprising one or more sets of (Gly-Ser)n residues, where n is a number between 1 and 10, such as 1-5. Exemplary linkers also can comprise ˜1-10 repeats of a sequence according to the formula GlyxSery, wherein x and y are each independently an integer from 1 to 5. In embodiments, linker(s) can comprise more glycine residues than serine residues, e.g., by comprising a (GxS)y sequence, wherein x and y are integers, wherein x=1, 2, 3 or 4, and wherein y=1, 2, 3, 4, 5, 6, or 7. In another exemplary aspect, an FP comprises a linker sequence comprising a (G-G-G-G-S)x, amino acid sequence where x is a positive integer. In another aspect FP(s) comprise linker(s) according to (GGGGS)n (SEQ ID NO:459), where n is 1, 2, 3, 4, 5, 6, 7, or 8. In aspects, 1, ≥2, primarily all, generally all, or all linkers of a FP encoded by a construct are at least 60% Gly in composition. In aspects, FP(s) comprise linkers comprising Ser residue(s), Thr residue(s), or both. In aspects, OSMGASAOA linkers of the FP(s) are mostly composed of, generally consist of, or consist of Ser/Thr linkers, in aspects such FPs exhibits DOS enhanced solubility via the linker(s). In aspects, FPs can be formed most, generally, or only from a mixture of Ala and Gly; Ser and Gly, or Ala, Gly, and Ser or from only Ala/Ser residues. E.g., a linker can comprise 11 Ala (e.g., 9-12 Ala residues) and about 5 Gly residues (4-6 Gly residues). Another example is a mixture of about 7 Ser (e.g., 5-8 Ser residues) and about 9 Gly residues (e.g., 8-10 Gly residues). In aspects, FP(s) comprise Ser/Thr linkers or Ser/Thr rich linkers, or both, which exhibit detectably enhanced, and in aspects DOS enhanced resistance to proteolysis.

In aspects, constructs of the invention encode fusion proteins comprising 1-10, 1-8, or 1-5 linkers, such as 2-24, 2-18, 2-16, 2-12, 2-10, 2-8, or 2-6, or 3-21, 3-15, 3-12, 3-9, or 3-6 linkers, which linkers comprise 2-32 residues, commonly 2-20 residues, often 3-21 residues, 4-20 residues, 4-16 residues, or 4-12 residues, in aspects ≥33% of which or ≥50% of which are Gly residues, and optionally ≥10%, ≥20%, or ≥33% of which are Ser, Thr, or a mixture thereof. Additional exemplary flexible linkers that can be in FPs include linkers comprising, GCO, or consisting of a sequence according to one of the following formulas: (GGGGS)n wherein n is 1-8, 1-6, 1-4, 1-3, 2-3, or 3; GGGS (SEQ ID NO: 460)), with n being 1-10, 1-6, or 1-3; SGGG (SEQ ID NO: 461))n with n being 1-10, 1-6, or 1-3; (Gly)₂₋₁₂, (e.g., Gly₄, Gly₃, Gly₆, and Gly₈). Specific examples of such linkers include SEQ ID NOs: 458-474 and SG, and linkers including CTs or repeats thereof (e.g., 2×, 3×, or 4×of any thereof). In aspects, FPs comprise linker(s) that comprises ≥1 Ala residues. In aspects, linkers are longer than 2 residues in length, such as Ala-Ala-Ala (A-A-A) or SEQ ID NO:475. In aspects, the fusion protein comprises a linker that comprises Ala residues and other AA types (see US20200325182).

In aspects, linkers in FPs PC, GCO, or CO linkers that are ≥3 AAs in length. In aspects, linkers of FPs are ≥4 AAs in length, e.g., ≥5 AAs in length. In aspects, the fusion protein comprises one or more linkers comprising Ala and at least one Glu (E) residue, at least one Tyr (Y) residue, or a combination thereof. Such linkers are typically considered rigid or at least relatively more rigid and associated with less rotation/free movement than the flexible linkers described above. Examples of such “rigid” linkers include linkers according to the formulas: (EAAAK (SEQ ID NO:476))₁₋₄, SEQ ID NO:477, and A(EAAAK)₁₋₄ALEA(EAAAK)₁₋₄A, e.g., SEQ ID NO:478. Linkers with similar compositions include SEQ ID NOs: 479 and 480. Other linkers can comprise Pro residue(s). Such linkers are also typically rigid or relatively rigid (e.g., as compared to a flexible linker). An example of such a linker is SEQ ID NO:481. In aspects, such a linker PC, GCO, or CO Gly & Pro AAs or Ala & Pro AAs. Longer Pro-containing linkers can form loop structures, which can be incorporated into FPs in aspects. An example of such a linker is SEQ ID NO:482, which forms a 281 degree turn loop. EPs associated with similar linkers that can form 90+, 120+, 150+, 180+, 210+, 240+, or 270+ degree turns are aspects.

Still other linkers comprise F, L, I, or V residues in combination with any of the residues discussed above. Examples of such linkers include G-F-L and G-F-L-G (SEQ ID NO:483) linkers, A-L-A-L (SEQ ID NO:484) linkers, V-A linkers, V—K linkers, V—K linkers, V-C inkers, and V—R linkers, and mixtures or repeats thereof (e.g., repeats of 2×, 3×, 4×, or 5× thereof or of a mixture of any thereof). Other examples of such linkers include SEQ ID NOs: 485-487 and GIG, and repeats/combinations thereof. In aspects, such linkers can further include an Arg residue (e.g., a RIG sequence linker). In aspects, linkers also may contain cysteine residues, for example, if dimerization of linkers is used to support or facilitate multimerization of a multimeric polypeptide (e.g., a Trap protein, examples of which are discussed further herein) into its properly folded configuration. Such linkers may also comprise a multimerization domain.

In certain embodiments, linker(s) comprise a glycosylation sequence. In certain embodiments, the linker comprises an amino acid sequence according to Asn-Xaa-Ser/Thr/Cys where Xaa is any amino acid or, in certain embodiments, wherein Xaa is any amino acid except Pro and Ser/Thr/Cys is serine, threonine or cysteine. In certain embodiments, the linker comprises the amino acid sequence Asn-Ala-Ser. In certain embodiments, the linker is a glycosylation sequence. In aspects, linker(s) comprise AARSs according to Asn-Xaa-Ser/Thr/Cys where Xaa is any AA or, in certain embodiments, wherein Xaa is any amino acid except Pro and Ser/Thr/Cys is serine, threonine or cysteine. In certain embodiments, the linker is the amino acid sequence Asn-Ala-Ser. However, in other aspects, most, generally all, or all linkers of FP(s) are free of any potential glycosylation sites. In aspects, constructs comprise a furin-sensitive linker (e.g., SEQ ID NO:488) or furin-resistant linker (e.g., SEQ ID NO:489). In aspects, linker(s) include linkers including glycosylation site(s) (e.g., SEQ ID NOs: 490-493). FPs can comprise one or more “linking means”, such as a “flexible linking means” or a “rigid linking means”, i.e., linkers provided herein and their equivalents.

In aspects, a gDAgFP or other FP EP will comprise ≥1 linkers that are at least 4 amino acids in length and that detectably enhances one or more antigen-specific immune responses to one or more Ags in the fusion protein or other expression product as compared to a corresponding fusion protein comprising only a 2-residue (two-mer) or 1-residue linker or lacking any linker.

In aspects, FPs comprise ≥1 cleavage sites that facilitate the separation of referenced sequences/domains. A cleavage site is a sequence that is specifically targeted and cleaved by an enzyme or a limited set of enzymes (a “cleavage partner”) (endogenous or introduced). FP(s) can comprise any suitable number of cleavage sites (aka, “cleavage signals”). For example, a fusion protein can comprise one, two, three, four, five, or more cleavage sites (e.g., 1-10, 1-8, 1-6, 1-3, or 1-2 sites). A cleavage site may be located at the N-terminus, the C-terminus, between the N- and the C-termini, e.g., about half-way between the N- and C-termini, between the N-terminus and the mid-point, between the halfway point and the C-terminus, or CT.

Cleavage sites of FPs may be any suitable type of cleavage sites. E.g., cleavage sites include proprotein convertase (or prohormone convertase), thrombin, and Factor Xa protein cleavage signals. Another example is a furin cleavage site. Examples of such cleavage sites include FIX-albumin (e.g., VSQTSKLTRIAETVFPDV, SEQ ID NO:494) (the down arrow indicating the point of cleavage); LAP-IFN-β; MazE-MazF (e.g., PLG↓LWA, EDVVCC↓SMSY, and GGIEGR↓GS) (SEQ ID NOS: 495-497), immunotoxin (e.g., TRHRQPR↓GWE, AGNRVRR↓SVG, RRRRRRR↓R↓R, and GFLG↓ (SEQ ID NOs: 498-501). An example of a factor Xa-sensitive cleavage site is SEQ ID NO:502. Examples of thrombin-sensitive cleavage sites include SEQ ID NOs:503 and 504.

In aspects, FPs encoded by constructs comprise ≥1 “self-cleavage” sites (“SCSs”), such as a 2A self-cleaving peptide site. SCSs can result in cleavage of an FP following expression without requiring the expression or co-administration of a cleavage partner. Although these sequences are known in the art as “self-cleavage” peptides, it is believed that they are likely cleaved through internal processes of cell(s). Regardless of how cleavage occurs with such sequences, a “self-cleavage” site herein means any cleavage site that is effective at specifically inducing the cleavage of the fusion protein at the site without the co-administration or co-expression of a cleavage partner/enzyme.

In one aspect the fusion protein comprises 2A cleavage site(s), such as 2, 3, 4, 5, or more cleavage sites. In aspects, one, some, most, or all of the 2A cleavage sites are selected from P2A, E2A, F2A and T2A cleavage sites (e.g., SEQ ID NOs:505-508, respectively). In aspects, OSMGAOA 2A sequences incorporated in FP(s) are a T2A or a P2A sequence. An exemplary T2A sequence is incorporated into some of the exemplary vector constructs described in detail in this description.

In aspects, a linker, such as a flexible linker, is added to the N terminus, C terminus, or both, of SMGAOA of the 2A sequences incorporated into FPs. E.g., in aspects, the sequence GSG is added to the N-terminus, C-terminus, or both of a 2A sequence, such as one of the sequences described in this paragraph. In aspects, a linker DOS enhances the rate of FP cleavage.

In aspects, self-cleavage site(s) also are associated with a 2^{nd} cleavage site that can be acted on by a cleavage partner. In one example, a FP comprises a 2A self-cleavage site in addition to a furin cleavage site. In such aspects, a method of the invention can comprise administering the cleavage partner or a composition can comprise a sequence encoding the fusion partner. In aspects, FP(s) comprise only one 2A site. In aspects, FP(s) comprise bicistronic, tricistronic, or quadcistronic 2A systems (see references included in US20200325182). Another type of self-cleavage sequence is an intein sequence, which provides polypeptide splicing-like functionality.

In aspects, EPs/consructs are multi-cistronic, at least in part, due to the incorporation of one or more IRES sites. In aspects, a construct comprises only one IRES element, such that the construct or vector is a bicistronic construct/vector. Examples of bicistronic vectors comprising IRES elements are described in, e.g., Santana V C, Diniz M O, Cariri F A, et al. Bicistronic DNA vaccines simultaneously encoding HIV, HSV and HPV antigens promote CD8+ T cell responses and protective immunity. PLoS One. 2013; 8 (8): e71322; and Harries M et al. J Gene Med. 2000; 2(4):243-249. However, in other aspects multiple IRES are contained in a construct to make or contribute to a multi-cistronic expression property of the construct. (See, e.g., Yeo J H M et al. Methods Mol Biol. 2018; 1827:335-349). In aspects, an IRES-comprising vector is a non-viral vector. In aspects, constructs incorporate two or more means for providing multi-gene/multi-cistronic expression, such as a splicing sequence and an IRES, an IRES and a 2A peptide, a bidirectional promoter and one or more 2A sequences, or multiple expression cassettes, etc.

EPES(s)/CS(s) of constructs can encode an immature protein that is subsequently processed in the cell/tissue where it is expressed. One typical element of an immature protein of functional significance a signal sequence (sometimes also called a “leader sequence” aka a secretory peptide/sequence or signal peptide). In aspects, a native leader sequence is expressed with an associated/homologous AAR sequence (e.g., where a gD leader sequence or highly homologous variant thereof is expressed along with other gD sequences). In other aspects, constructs of the invention include a heterologous leader sequence (with respect to the associated mature protein). E.g., an EP can be associated with a human tissue plasminogen activator (TPA) leader sequence or mouse β-glucuronidase leader sequence.

Constructs can also encode FP(s) including targeting domains/sequences (which also sometimes and in some contexts are referred to as “localization sequences”, “targeting domains”, and the like), distinct from signal sequence(s), but which also can direct the transport of the associated polypeptide, processing of the polypeptide, or both, in a manner unlike that of a signal sequence, such as by directing associated PPTs to certain organelles within a cell or to other cells in a TR/tissue.

Distinctions between typical signal sequences and other targeting sequences can include targeting of a specific organelle outside of the secretion organelles, targeting of particular receptors (and thus, cells), often having larger or smaller lengths than typical signal sequences (less than the 15-30 amino acid length associated with most signal peptides or more than about 30 amino acids in some cases), placement of the targeting sequence outside of the N-terminus of the polypeptide, lack of cleavage from the polypeptide, or a combination of any or all thereof. Thus, such distinctions may not be binding.

EP(s) can comprise targeting sequences (TS(s)) (e.g., PTPS sequences and gD sequences that bind to particular receptors). FPs expressed by constructs included in compositions of the invention can also, typically alternatively, target any other suitable target, whether intracellularly or extracellularly. FPs expressed by constructs in CEPESCs that comprise TS(s) other than gD RBD sequences can still include gD sequences (e.g., a gD profusion domain sequence, a gD signal sequence, or both) or such fusion proteins can lack any gD sequences, e.g., where such a fusion protein is encoded by a second NAM of a composition and expressed in combination with a gD-antigen fusion protein. Examples of non-gD extracellular molecules that can be bound by non-gD targeting sequences included in fusion proteins expressed by constructs of the invention include, e.g., include MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM⁻¹, Fcγ receptor, T cell receptor, or lectin, or any of the other various immune cell receptors described herein. In aspects, a composition comprising sequences encoding a gD-antigen fusion protein and a non-gD targeting sequence: antigen fusion protein (e.g., a DEC-205-binding: antigen fusion protein expressed by one NAM and a gD-antigen fusion protein expressed by a second NAM of a composition).

A targeting sequence can target an intracellular target, e.g., an organelle (e.g., the ER or the proteasome) or an extracellular target, e.g., a receptor on a cell that is different from COE (e.g., a dendritic cell receptor). Compositions can comprise NS(s) encoding or more of each type of targeting sequence or a combination of such targeting sequences in combination with other AARS(s) (e.g., in one aspect a fusion protein comprises (a) at least one extracellular targeting sequence, such as dendritic cell receptor-binding sequence, which can be a gD sequence or a heterologous dendritic cell receptor binding sequence, and (b) at least one intracellular targeting sequence, such as a PTPS or endoplasmic reticulum targeting/processing sequence (ERTPS)). A fusion protein can also comprise a signal sequence in addition to an intracellular targeting sequence, extracellular targeting sequence, or a combination thereof.

For example, a vector of the invention, e.g., a viral vector, can include a nucleotide sequence encoding a genetically engineered antigen receptor, such as a chimeric antigen receptor (CAR) (vector targeting is also discussed elsewhere herein). Other examples of localization sequences that can be encoded by constructs of the invention include, e.g., a nuclear localization sequence, an endoplasmic reticulum (“ER”) localization sequence, a peroxisome localization sequence, or a mitochondrial localization sequence (see, e.g., “Protein Targeting”, chapter 35 of Stryer, L., Biochemistry (4th ed.). W. H. Freeman, 1995). Examples of localization sequences include those targeting the nucleus (e.g., SEQ ID NO:509), mitochondrion (SEQ ID NO:510), endoplasmic reticulum (KDEL (SEQ ID NO:511)), peroxisome (SKF), prenylation or insertion into plasma membrane (CAAX (SEQ ID NO:512), CC, CXC, or CCXX (SEQ ID NO:513)), cytoplasmic side of plasma membrane (fusion to SNAP-25), or the Golgi apparatus (fusion to furin). Polypeptides expressed from constructs of the invention can include an intracellular targeting sequence (or “sorting signal”) that directs the polypeptide to an endosomal and/or lysosomal compartment(s), for example a compartment rich in MHC II to promote CD4+ and/or CD8+ T cell presentation and response, such as a lysosomal/endosomal-targeting sorting signal derived from lysosomal associated membrane protein 1 (e.g., LAMP-1 (see references cited in US20200325182)). Additional targeting sequences include ER targeting/processing sequences such as SEQ ID NO:511), lysosomal targeting sequences, such as SEQ ID NO:514, or other ER or Golgi retention sequences having a structure according to the formula KKXX (SEQ ID NO:515) or KXD/E (where X can be any AAR) or comprising the SEQ ID NO:516 (see Zhan J, et al, Cancer Immunol Immunother 46:55-60, 1998).

In aspects, TS(s) allow a FP to DOS bind target(s) and remain detectably associated with the target or processed by the target (e.g., taken into a cell) under relevant physiological conditions (e.g., at a pH of ˜7-8 and a temperature of −37° C., such as at a temperature of from ˜20° C. to ˜40° C. (for example, room temperature), and a pH of about 7.5, or other suitable combination of temperature, pH, and other conditions) for a significant period of time (e.g., ≥˜30 minutes, ≥˜45 minutes, ≥˜1 hour, ≥˜4 hours, ≥˜8 hours, or longer such as about 1-12 hours, about 1-24 hours, about 1-36 hours, about 1-48 hours, about 1-72 hours, etc.)). In aspects, the targeting sequence specifically binds a fusion protein to its target.

In aspects, TS(s) do not exhibit DOS binding of other sequences in the FP, other than the binding of between any multimerization domains of the fusion protein sequence, in which each expressed polypeptide chain of a fusion protein results in a multimeric fusion protein. In aspects, FP(s) forms or is part of a multimeric peptide. In other aspects, FP(s) lack(s) any multimerization domains. Like other constituent sequences, targeting sequence(s) also typically does not, due to its composition (rather than function) result in a DOS immune response or does not result in an initial immune response that is considered clinically significant in view of the therapeutic effect of the CEP. Also, as with other components of a fusion protein, the targeting sequence(s) of the fusion protein do not DOS interfere with the functioning of the other components of an FP/CEP (e.g., the promoting, induction, or enhancement of IR(s)).

In aspects, a fusion protein can be characterized as being multivalent, multi-specific, with respect to extracellular targets, intracellular targets, or both types of targets, and also can be monomeric (single chain) or multimeric (formed from the association of two or more polypeptide chains, typically two or more expression products of a composition of the invention, and, accordingly, having a quaternary structure). Valency refers to the number of binding sequences/regions/domains in a biomolecule and, accordingly, provides an indication of the maximum number of targets/molecules that can be bound by an FP or other molecule (e.g., an antibody). Specificity refers to the ability to specifically bind one or more targets (a bi-specific antibody, for example, exhibits specific binding to 2, typically heterologous, targets).

In aspects, a multimeric FP (e.g., a multivalent multimeric fusion protein) exhibits detectably or significantly increased binding for one or more targets than a corresponding monomeric polypeptide. In aspects, a multimeric FP or other EP exhibits DOS enhanced IR(s), such as more rapid antigen presentation, a more rapid B cell response, a more rapid T cell response, or a combination of any or all thereof.

In aspects, the immune cell targeting sequence can be characterized as specifically binding to one or more such immune cell receptors (specifically examples of such receptors are provided below). The term “specific binding” or “specifically binds to” or is “specific for” a particular polypeptide can be exhibited by the binding sequence detectably or significantly binding the referenced binding target (e.g., the immune cell receptor) preferentially as compared to any other polypeptides in the environment. In aspects, “specific binding,” means binding between one agent, e.g., an antibody or T cell receptor (“TCR”) and a second agent, e.g., a respective epitope, of ˜1×10⁵ M⁻¹ or stronger, or of ˜1×10⁶ M⁻¹, ˜1×10⁷ M⁻¹, or ˜1×10⁸ M⁻¹ or stronger (PMCs provided in, e.g., Caoili, S. E. (2012) Advances in Bioinformatics Vol. 2012). In aspects, a part of an FP that binds to a partner will bind that partner with essentially equivalent (+/−15%) affinity, avidity, speed, duration, etc. of WTC(s) (typically the closest related WT sequence/PPT). In other aspects, FP(s) bind to partner(s) with DOS enhanced binding affinity, avidity, speed, duration, or combination of any or all thereof, such as an affinity that is at least about 15% greater, ≥25% greater, ≥33% greater, ≥50% greater, or ≥100% greater (i.e., about 2× in value, such as ≥2.5×or ≥3×in value) the affinity, avidity, speed (Kₒₙ), duration (K_{off}), or combination of any or all thereof, of one or more corresponding wild-type proteins.

The principles described in this section relating to affinity and binding relationships between sequences can be applied to any other association described herein between sequences and binding partners (e.g., the association of two or more sequences that form a multimeric trap polypeptide expressed by a construct of the invention; the association between an epitope of the invention and an immune cell epitope recognition receptor; the association between a cytokine and a cytokine receptor; etc.).

In aspects, specific binding further means that the binding sequence does not bind a significant amount of any other polypeptide in the environment. In a particular aspect, specifically binding means that the binding sequence binds the indicated target or targets and does not detectably bind other polypeptides in the relevant environment.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X (e.g., a FP comprising one or more receptor binding sequences) for its partner Y (e.g., a receptor of an immune cell) can generally be represented by the dissociation constant (“Kd”). E.g., a Kd can be about 200 nM, 150 nM, 100 nM, 60 nM, 50 nM, 40 nM, 30 nM, 20 nM, 10 nM, 8 nM, 6 nM, 4 nM, 2 nM, 1 nM, etc. In aspects, TS(s) or receptor binding domain(s) (RBD(s)) of EP(s) exhibit such levels of affinity for target(s). Affinity can be measured by common methods known in the art. Methods are described in US20200325182.

In aspects, an “effectively binding” or “specifically binding” relationship also can be characterized based on the binding sequence exhibiting a Kd for the target of at least about 200 nM, ≥150 nM, ≥100 nM, ≥60 nM, ≥50 nM, ≥40 nM, ≥30 nM, ≥20 nM, ≥10 nM, ≥8 nM, alternatively ≥6 nM, ≥4 nM, ≥2 nM, or ≥1 nM, etc. In aspects, TS(s) or RBD(s) of EP(s) exhibit such levels of affinity for their target(s).

In aspects, an “effective binding” or “specifically binding” relationship can also mean that the referenced molecule binds its partner with a Ka (an equilibrium association constant) of greater than a threshold, such as equal to or greater than about 10⁵ L·mol⁻¹ (or “M”)⁻¹. Ka reflects the speed of association between two molecules and is the inverse of Kd. In aspects, a fusion protein will exhibit “high affinity” for a target, by exhibiting a Ka of ≥10⁷ M⁻¹, ≥108 M⁻¹, ≥10⁹ M⁻¹, at least 10¹⁰ M⁻¹, at least 10¹¹ M⁻¹, at least 10¹² M⁻¹, or at least 10¹³ M⁻¹. constant) for the target antigen.

In still another aspect, an “effective binding” or “specific binding” relationship also means that the K_{off} with respect to the fusion protein or other test product and the target is less than about 1×10⁻⁵ s⁻¹, such as ≤˜5×10⁻⁴ s⁻¹, such as ≤˜1×10⁴ s⁻¹, such as ≤˜5×10⁻³ s⁻¹ or ≤˜ 1×10⁻³ s⁻¹. In aspects, the fusion protein binds its target with a K_{off} of from about 1×10⁻⁴ s⁻¹ to about 1×10⁻² s⁻¹, such as about 1×10⁴ s⁻¹ to about 5 ×10⁻² s⁻¹ or about 1×10⁴ s⁻¹ to about 1 ×10⁻³ s⁻¹. In another aspect “effective binding” or “specific binding” can also comprise binding with a Kₒₙ of more than about 0.01 M⁻¹S⁻¹, ≥˜0.1 M⁻¹S⁻¹, ≥˜1 M⁻¹S⁻¹, or ≥˜5 M⁻¹S⁻¹. In aspects, TS(s) or RBD(s) of EP(s) exhibit such affinity characteristics for target(s).

In aspects, FP(s) comprise 1+TS(s) of an Ab against the target compound (partner/target) a variant of such an antibody sequence that is RVRHROSI to one or more antibody sequence(s) that bind to the target.

An antibody against a referenced target herein, generally means any suitable type of antibody molecule that specifically binds to one or more portions of the referenced target, under cellular and/or physiological conditions for an amount of time sufficient to allow detection of such binding by suitable means (e.g., ELISA, Western blot, or other similarly suitable protein binding technique described herein and/or known in the art) and, typically, to detectably or significantly promote, induce, or enhance a cellular or physiological effect associated with such binding. The term antibody thus includes any suitable type of immunoglobulin molecule, a fragment of an immunoglobulin (e.g., a fragment that retains a target-binding fragment of an antibody molecule) that specifically binds to a specific target. An immunoglobulin ion can be of any type/isoytpe (e.g., IgG, IgE, IgM, IgD, IgA, and IgY), class (e.g., IgG1, IgG2, IgG3, IgG4, IgA1, and IgA2) or subclass of immunoglobulin molecule. Target-binding functions of antibodies can be performed by any number of suitable “fragments” thereof. An antibody fragment can, for example, lack one or both constant domains of an antibody molecule. Examples of typically suitable antibody fragments include (i) a Fab fragment, a monovalent fragment consisting essentially of the VL, VH, CL and CH I domains; (ii) F(ab)₂ and F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., (1989) Nature 341:544-546), which consists essentially of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain antibodies or single chain Fv (scFv). In aspects, antibody molecules can be linear antibodies, which comprise a pair of tandem Fd segments that form a pair of antigen binding regions (such antibodies can be bispecific or monospecific) (SFE Zapata et al. Protein Eng. 8(10):1057-1062 (1995). Any of these antibody “fragment” or antibody derivative molecules also are encompassed within terms such as antibody fragment and should be considered implicitly referenced as a separate aspect of any part of this disclosure that makes reference to the use, expression, or presence of an antibody in a composition or method of the invention, unless contradicted (the term “fragment” is not meant to indicate any means of obtaining such an antibody molecule that lacks all of the components of a full length antibody). E.g., FPs comprising antibody targeting sequences can comprise antibody “fragments” including either a VL or VH domain or an effective combination of CDRs or even an effective epitope-binding CDR. In cases, inclusion of substantially all or all of an Ab CDR3, e.g., may be sufficient to impart effective target binding. SFE Zhong G S et al. Oncol Lett. 2013; 5(4):1183-1188 regarding minimized antibody fusion proteins.

FPs can comprise any suitable combination of Ab sequences that facilitate binding of referenced target(s). In aspects, a fusion protein of the invention comprising antibody target binding sequences will result in a single chain fusion protein. Single chain antibody fusion proteins and related techniques and principles applicable to generation of such fusion proteins are exemplified in, e.g., Zaneti A B et al. Front Immunol. 2019; 10:59; Ahmad Z A et al. Clin Dev Immunol. 2012; 2012:980250; De Vlaeminck Y et al. J Control Release. 2019; 299:107-120; US20020018783A1, US20190202931A1, and U.S. Pat. No. 5,990,296. In aspects, FP(s) comprising antibody targeting sequences will comprise a multimerization domain, typically an antibody multimerization domain, thereby forming a multimeric, often multivalent (e.g., bivalent, trivalent, or tetravalent), and sometimes multi-specific (e.g., bispecific) antibody fusion protein(s) (Ab FP(s)). Examples of multivalent/multimeric antibody fusion proteins and related principles are described in, e.g., WO2011036460A1, WO2019129053A1, and US20180216093A1.

Antibodies and other compositions (e.g., antigens, cytokines, adjuvants, vectors, linkers, and the like) described in the references cited in the preceding paragraph also can be components of combination therapy/immunization aspects and combination compositions.

In aspects, antibody fusion proteins can exhibit one or more DOS “antibody effector functions,” such as Clq binding; complement dependent cytotoxicity; Fc receptor binding; Ab-dependent cell-mediated cytotoxicity (ADCC); detectably inducing/enhancing phagocytosis, down regulation of cell surface receptors (e.g., B cell receptor (BCR)); cross-presentation of antigens by antigen presenting cells/dendritic cells; or a combination of any or all thereof. In aspects, an antibody targeting sequence fusion protein will exhibit two or more of such functions. In other aspects, a fusion protein comprising one or more antibody targeting sequence will exhibit no effector functions. In aspects, FPs comprise one or more targeting sequences that are non-antibody sequences, having no identity or substantial identity to any known epitope-binding antibody sequences for the target or, in aspects, for any target. In aspects, fusion proteins of the invention lack any antibody targeting sequences other than CDR and FR sequences. In aspects, a fusion protein of the invention lacks any antibody targeting sequences. In aspects, a fusion protein of the invention lacks any antibody sequence (e.g., comprises no sequence of more than about 50 amino acid residues, ≥˜35 amino acid residues, or even ≥˜25 amino acid residues exhibiting substantial identity to an antibody sequence).

A fusion protein expressed by a construct of the invention can comprise any suitable number of ligand/receptor sequences. In aspects, a fusion protein comprises only a single extracellular targeting domain (e.g., a gD receptor-binding domain that binds to a single gD receptor in a target species, such as Nectin-1). In other aspects, a fusion protein can comprise one or more sequences that bind to two or more extracellular targets, such as a single gD sequence that binds to multiple receptors (e.g., HVEM and Nectin-1) or two heterologous sequences that target different receptors (e.g., a DEC-205-binding keratin sequence and a HVEM-binding gD sequence). In any such case, such a fusion protein can also comprise one or more intracellular targeting domains (e.g., a PTPS, an ERTPS, or both).

Non-antibody targeting sequences can comprise any portion of a polypeptide that specifically binds to a target. Binding sites from a receptor-ligand pair are non-limiting examples of domains that, in a manner akin to an antibody-epitope interaction, may be used as targeting sequences, similar to how in aspects of the invention a receptor-binding gD domain may be incorporated to bind a gD polypeptide to a gD receptor (e.g., Nectin-1). Further exemplary non-antibody targeting sequences include antibody mimetics, such as polypeptide scaffolds that mimic the structure of an antibody. FPs can comprise any suitable ligand binding domain, any suitable receptor binding domain, or a combination thereof, which act/acts as a targeting sequence in the fusion protein. A receptor binding domain (RBD) is a portion of a ligand molecule that specifically binds to a site on a receptor.

In aspects, the targeting domain comprises a sequence that is a ligand for a receptor of a cell of the immune system. In aspects, the targeting domain is a ligand for a cell of the innate immune system (e.g., a macrophage), the innate trained immune system (e.g., an NK cell or dendritic cell), or the trained/adaptive immune system (e.g., a T cell). Targeting sequences can comprise at least a ligand binding fragment of, e.g., a T lymphocyte immunoreceptor, T cell inhibitory receptor (TCIR), T-cell co-inhibitory molecule, T-cell co-stimulatory molecule, B lymphocyte receptor, DC receptor, NK cell receptor, cytokine receptor, growth factor receptor, chemokine receptor, or tumor cell receptor. Targeting sequences also can comprise sequences that effectively bind to a component of a tumor cell, tumor microenvironment, tumor associated growth factor or receptor, tumor associated cytokine or receptor, tumor associated T lymphocyte, T cell co-stimulatory or inhibitory molecule, immune cell, pathogen, or pathogen-associated cell. Specific examples of ligand and receptor sequences that can be used as targeting sequences include, e.g., binding sequences for binding or binding sequences of cytotoxic T lymphocyte associated antigen-4 (CTLA-4, CD 152), Programmed Death-1 protein (PD-1), Programmed death ligand-1 (“PD-L1” or “PDL-1”), Programmed death ligand (PD-L2 or PDL-2), B7-H3 (CD276), T-cell immunoglobulin and mucin-domain containing-3 (TEVI-3), Carcinoembryonic antigen-related cell adhesion molecule 1 (CEACAM⁻¹), Carcinoembryonic Antigen (CEA), V domain Ig suppressor of T cell activation (VISTA), V-set and immunoglobulin domain containing 8 (VSIG8), B and T lymphocyte attenuator (BTLA), Herpesvirus Entry Mediator (HVEM), CD 160, T cell Ig and ITM domain (TIGIT), CD226, CD96, Lymphocyte activation gene-3 (LAG-3), transforming growth factor β (TGF-β), transforming growth factor β receptor (TGFpR), Receptor Activator of Nuclear Factor κ B (RANK), RANK ligand (RANKL), 4-IBB (CD137), Inducible T-Cell Costimulator (ICOS), OX-40 (CD134), glucocorticoid-induced TNFR-related protein (GITR), CD27, IL6R, IL23R, IL17R, IL-6, IL-23, IL-17,CD39, CD40, CD40L, CD47, CD73,CCR4, CCR5, CXCR4, IL12R, CD4, IL-2R, CD25, CD3, gp120, NKG2D, Epidermal growth factor receptor (EGFR, EGFR1, ErbB-1, HER1), ErbB-2 (HER2/neu), ErbB-3/HER3, ErbB-4/HER4, Epidermal growth factor (EGF), Transforming growth factor a (TGFa), Vascular endothelial growth factor (VEGF), Vascular endothelial growth factor receptor-1 (VEGFR-1), VEGFR-2 or VEGFR-3, transforming growth factor β receptor II (TGFpRII), programmed death 1 protein (PD-1), T cell immunoglobulin and mucin domain containing 3 (TEVI-3), B- and T-lymphocyte attenuator (BTLA), CD 160, CD226, T cell Ig and ITM domain (TIGIT), CD96, CD44, Colony stimulating factor 1 receptor (CSF1R), CCR4, Killer-cell immunoglobulin-like receptor (KIR), Vascular endothelial growth factor receptor (VEGFR), Receptor Activator of Nuclear Factor κ B (RANK), V-set and immunoglobulin domain containing 8 (VSIG8), LIGHT (TNFSF14), Leukocyte Associated Immunoglobulin-like Receptor 1 (LAIR1), or V domain Ig suppressor of T cell activation (VISTA).

In aspects, FPs comprise one or more multimerization domains and will be or will ultimately form a multimeric protein after expression. Multimeric fusion proteins will typically exhibit binding preference for other expression products of the constructs of a composition as compared to other endogenous proteins, except in aspects for targets bound by targeting sequences where the targeting sequence is operable in a single chain expression product. In aspects, the multimeric protein will be multivalent (comprising two or more binding domains), and possibly multi-specific, binding to two or more different targets, typically with different binding properties (e.g., affinities). In aspects, such a multimeric PPT binds a single type of target or single target. In aspects, such a multimeric PPT is multivalent, comprising at least two domains that contribute to the binding of the single type of target or single target.

In aspects, FP(s) are multimeric. A multimeric FP typically will comprise at least one suitable multimerization domain. A “multimerization domain” is a sequence/domain that preferentially interacts or associates with another polypeptide molecule or region, directly or indirectly, wherein the interaction of multimerization domains substantially contribute to or efficiently promote multimerization (i.e., the formation of a dimer, trimer, tetramer, or higher order multimers, which may be a homodimer, heterodimer, homotrimer, heterotrimer, homomultimer, heteromultimer, or the like). Multimeric PPT EPs may be multi-specific, if they bind to more than one target (e.g., DEC-205 and HVEM). Multimeric fusion proteins also can be multivalent, comprising two, three, four, or more binding domains for one, two, three or more extracellular targets, intracellular targets, or a combination thereof. A multimeric FP can comprise any suitable type of multimerization domain(s). Exemplary multimerization domains comprise ≥1 disulfide bonds, zinc finger motif(s), leucine zipper motif(s), helix-turn-helix motif(s), helix-loop-helix motif(s), etc. In aspects, a multimeric protein is formed by i.a., ≥1 cys-cys bond(s). Multimerization means the association of at least two multimerization units and more typically three, four, five or six such units via association of a homomultimerizing peptide. In aspects, multimers comprise at least two initially separate polypeptide chains that form multimers after initial expression. Single chain multimeric peptides also can form through intra-chain domain: domain interactions, such as in some single chain antibody sequence fusion proteins (described above). A multimerization domain can be, e.g., a dimerizing, a trimerizing, a tetramerizing or a pentamerizing multimerization domain. In aspects, multimers are connected without any intervening amino acids. In aspects, a multimer comprises a linker sequence, such as a linker sequence of about 1 to about 250, or ˜1 to ˜100, or ˜1 to ˜50, ˜1 to ˜25, ˜1 to ˜15, ˜1 to ˜10, or ˜1 to ˜5 amino acids. E.g., PPTs can comprise ≥2 PPT chains expressed from constructs. In some cases, two PPT chains in an EP are covalently linked to one another, e.g., via a disulfide bond. In other instances, two PPT chains of an EP are not covalently linked to one another.

In aspects, a multi-chain, multimeric fusion protein final product is achieved from the expression of an initial single chain fusion protein comprising two or more multimerization domains that lead to multimer formation and at least one cleavage site, such as a 2A cleavage site, which cleavage site can be optionally located in a linker, and which cleavage site ultimately results in the cleavage of the initially expressed polypeptide chain, at some of the fragments of which can assemble into multi-chain, multimeric fusion proteins. In aspects, FPs expressed from NAM(s) comprise a mixture of monomers and multimers.

As noted, FPs can include Ab multimerization domains. Multimerization through CH2 and CH3 domains of IgG derived proteins is described in, e.g., Soleimanpour et al, 2015, Appl Microbiol Biotechnol). Fab-fragment multimerization approaches are described in, e.g., Mayer et al, 2015, Int J Mol Sci). Several references describe the use of antibody Fc regions to form multimeric proteins. Any suitable one of these methods can be employed to generate multimeric fusion proteins of the invention.

In aspects, the multimerization (or “interaction”) domains/sequences of a multimeric fusion protein of the invention comprise, primarily comprise, generally consist of, or consist of non-antibody (non-immunoglobulin) domains. Thus, in aspects, the invention provides constructs that express multimeric fusion proteins that lack any antibody multimerization domains or that even lack any antibody sequences. Interaction (non-covalent multimerization) domains may correspond to or be derived from proteins that dimerize or multimerize, e.g., through non-covalent bonds and/or disulfide bonds, to form the quaternary structure of a protein. In aspects, the interaction domains of polypeptides expressed from constructs of the invention may be identical, so that homodimers are formed. In other aspects, the interaction domain monomers may be different, resulting in formation of heterodimers. Dimerization motifs from specialized proteins may be used. Exemplary, non-limiting proteins containing dimerization motifs include but are not limited to receptor tyrosine kinases, transcription factors such as leucine zipper motif proteins. coil-coil homodimerization motifs, fibronectin domains, etc.

Another class of multimeric proteins are “trap” proteins. A “trap” PPT/FP here refers to an EP that (1) binds and detectably or significantly modulates the biological activity of a single target molecule (e.g., through target-mediated cellular uptake of the fusion protein, downstream signaling, and the like), which target molecule typically is an extracellular molecule, such as a cell receptor (e.g., an immune cell receptor) and (2) comprises two or more identical or substantially identical (typically identical) polypeptide chains, each of which chain contains (a) a target binding sequence, (b) an optional but typically present flexible linker, and (c) a multimerization domain that is smaller than a full antibody multimerization domain, subject to less processing than a full antibody multimerization domain, or both, and which typically lacks any antibody multimerization sequences. For example, trap proteins lack Fc portions and do not exhibit effector functions. Compared to full-length Abs, trap proteins typically exhibit detectably better penetration of cells & microenvironments (cells & surrounding milieu material). As a trap protein is composed of identical monomers it is multivalent but monospecific.

In aspects, the trap protein comprises a non-antibody sequence multimerization domain. In aspects, the trap is a trimeric protein comprising a stable trimerization domain. An example of a suitable domain for formation of a trap protein is the trimerization domain from human CMP-1. Incorporation of a CMP-1 multimerization sequence results in a parallel, disulfide-linked, and rod-shaped trimeric structure with high stability following expression. The trap monomers can self-assemble into the final form of the multimeric trap protein in vivo, including in mammals. Other multimerization domains that exhibit similar stability and functioning can alternatively be used to form a trap protein, including variants of CMP-1 multimerization domains.

In aspects, the target binding domain of a trap protein can comprise antibody sequences, such as immunoglobulin VH domain, immunoglobulin VL domain, a VH and VL fusion protein, scFv, a PPT derived from a binding and/or framework region of an Ab. The target binding domain also can be a non-Ab target-binding domain, such as a single domain Ab mimic based on a non-immunoglobulin scaffold (such as an FN domain-based monobody, Z domain-based affibody, DARPINs), singly and in any combination thereof.

Sequences encoding a variety of multimeric trap proteins are described in this disclosure for incorporation into compositions of the invention. One example of a trap expressed by constructs of the invention is a DEC-205 binding trap, which can be a trimeric trap comprising, e.g., a CMP-1 trimerization domain, such as a murine or human CMP-1 trimerization domain, and anti-DEC-205 antibody receptor epitope binding sequences or DEC-205 ligand sequences, such as keratin sequences discussed elsewhere herein. In aspects, a trap comprising a CI AARS, such as a PD-L1 or PD-1 sequence is expressed from the nucleic acid sequences of a composition.

Trap proteins exhibit detectably or significantly greater binding for targets than their constituent monomers, enhanced stability as compared to constituent monomers, or both. In aspects, the trap exhibits at least about 20×, ≥50×, ≥100×, ≥200×, or ≥500×affinity for the target than its monomeric components. The trap construct does not significantly or detectably enhance the immunogenicity of the fusion protein or impair the functionality of the sequences contained in each chain. Each chain of such a trap can comprise, e.g., one or more antigenic sequences that are taken up by a target cell upon target receptor binding, as well as other components (e.g., one or more intracellular targeting sequences such as one or more PTPSs, ERTPSs, or a combination thereof). Exemplary trap proteins are described in Song W et al. Nat Commun. 2018; 9(1):2237.

Trap proteins that modulate targets can be classified as inhibitory traps or stimulatory traps. Inhibitory traps for macromolecule targets include traps that can be protein molecules that specifically bind and further inhibit or block the biological functions of a target of interest, such as an interleukin, TNF-alpha (and TNF-alpha receptor), TGF-beta (and TGF-beta receptor), CSF-1 (and CSF-1 receptor), CXCR proteins and ligands, CCR proteins and ligands, ACKR3 and its ligands (CCL11, CCL12), ACKR6 and its ligand (CCL18), CTLA-4, PD-1, PD-L1, PD-L2, B7-H3 (CD276), B7-H4 (VTCN1), HVEM (CD270 or TNFRSF14), BTLA (CD272), TIM-3, GALS, TIGIT, A2aR, LAG-3, KIRs and MHC class I or II. Traps can also be stimulatory, including those that agonistically act on immune checkpoint targets such as CD28, ICOS (CD278), 4-1BB (CD137 or TNFRSF9), OX40 (CD134 or TNFRSF4), GITR (CD357 or TNFRSF18), CD27 (TNFRSF7), and CD40 (TNFRSF5) or that mimic the agonistic effects of the ligands of the above receptors, including but not limited to B7.1 (CD80), B7.2 (CD86), B7-H5 (VISTA or Gi24), ICOSL (B7H2 or CD275), 4-1BBL (CD137L), OX40L (CD252), GITRL, CD27L (CD70), and CD40L (CD154). Targets for agonistic traps also include some toll-like receptors, including TLR4, TLR7, TLR8, & TLR9. Such trap proteins are described in US20190381184.

In aspects, the invention provides fusion proteins comprising a trap protein in which one or more antigens, such as two or more antigens, such as three or more antigens, are inserted within a multimerization domain, such as a trimerization domain (and constructs encoding such fusion proteins). In aspects, such antigens are bound to each other, the parts of the associated multimerization, or both, by linker sequences, such as those described elsewhere herein. In aspects, some, most, generally all, or all of such antigen sequences are associated with one or more intracellular targeting sequences, such as one or more PTPSs. Such FPs can target a DCR, such as DEC-205, and comprise a multimerization domain, e.g., a CMP-1 multimerization domain.

In cases, FP(s) can comprise targeting sequence(s) other than signal peptides. In aspects, FP(s) comprise intracellular targeting sequence(s). In aspects, a fusion protein comprises one or more phagosome targeting sequences. In one such aspect, the inclusion of the phagosome targeting sequence results in enhanced antigen-specific immune responses to one or more antigens included in the fusion protein or co-expressed with the phagosome-targeting sequence fusion protein. Phagosome targeting sequences have been described in the art (SFE Pereira M P et al. ACS Infect Dis. 2015; 1(12):586-592) and include cathepsin fragments, y-Secretase-targeting sequences (or sequences targeting related peptides CD44 or LRP), Rab39-binding sequences, and the like.

An intracellular targeting/localization and processing sequence also can be a sequence that enables an associated fusion protein to avoid, disrupt, or overcome processing by one or more organelles. For example, a fusion protein of the invention can comprise one or more lysosomal or phagosomal avoidance/disruption sequences. Such sequences can be obtained from adenovirus penton proteins, influenza virus HA-2, and the like (SFE US20020155609A1; Bal et al., Eur J Biochem 267:6074-81 (2000); and Wagner, et al., Proc. Natl. Acad. Sci. USA, 89:7934-7938, 1992).

In aspects, a fusion protein also comprises a sequence targeting an exosome. Examples of exosome targeting sequences, e.g., the N terminus of lysosomal associated membrane protein 2b (Lamp2b) (SFE Hung M E et al. J Biol Chem. 2015; 290(13):8166-8172 and Tian Y et al. Biomaterials. 2014; 35(7):2383-2390) and the C1C2 domain from MFG-E8.

In aspects, a fusion protein expressed by a construct of the invention also comprises an intracellular TS, extracellular TS, or both, which DOS promotes processing of the fusion protein by early endosomes (e.g., by incorporation of CD40-specific monoclonal antibody sequences). In aspects, a composition of the invention also comprises an intracellular TS, extracellular TS, or both, which, i.a., detectably or significantly targets late endosomes, early lysosomes, or both (e.g., a DEC205-specific sequence). In aspects, a composition of the invention comprises sequences encoding both types of such fusion proteins. In aspects, such different fusion proteins are encoded by sequences on different NAMs. In aspects, a method is provided in which such different NAMs are administered in association with each other (e.g., in a sequential administration method). In aspects, one of such types of fusion proteins comprises one or more gD sequences, one or more antigens, or both, or both of such types of fusion proteins comprise one or more gD sequences, antigenic sequences, or both.

In aspects, constructs encode FPs including proteasome targeting/processing sequence(s) (“PTPS(s)”). A PTPS can target any suitable type of proteasome or two or more types of proteasomes (e.g., a classical proteasome, an immunoproteasome, or a thymo-proteasome) (SFE Rock K L. Trends Immunol. 2016; 37(11):724-737 and Sijts E J et al. Cell Mol Life Sci. 2011; 68(9):1491-1502) and any suitable portion/assembly/aspect of a proteasome or proteasomal system (e.g., a 26S proteasome, 20S proteasome/chamber, a 30S proteasome, or hybrid proteasome, or further subunits such as a base subcomplex, a 19S core, or an RP complex) (SFE Tanaka K. Proc Jpn Acad Ser B Phys Biol Sci. 2009; 85(1):12-36, with respect to the biology and composition of proteasomes). In aspects, FPs comprise PTPS(s) that target an immunoproteasome (optionally in combination with a standard proteasome targeted by the same PTPS or another PTPS contained in the fusion protein). In an exemplary aspect, at least one PTPS of a fusion protein binds to one or more subunits of an immunoproteasome, such as β1i, β2i, or β5i. In aspects, at least one PTPS of a fusion protein also detectably or significantly binds to a thymo-proteasome or a subunit thereof (e.g., 1i, β2i, or 05t).

In aspects, a FP or other PPT comprises ≥1 proteasome targeting chaperon sequence, e.g., a calreticulin sequence (see, e.g., U.S. Pat. No. 9,085,638 and US20190177733). Calreticulin sequences in aspects promote DC uptake of PPTs/FPs, MHC I molecule association of antigens, induces immune cell cytokine production, promotes TH cell activity, or provide an “eat me” signal to DCs and other innate trained immunity/innate immunity cells. SFE Cheng W F, et al. J Clin Invest 2001; 108:669-678 and Gardai et al. Cell 123: 321-334, 2005). In aspects, such a construct further comprises ≥1 other types of PTPSs, e.g., 1+ ubiquitin sequences.

In aspects, a PTPS is a proteolytic signal or motif that targets an associated polypeptide for processing in the proteasome. Accordingly, fusion proteins of the invention can comprise one or more of such PTPSs or one or more of such PTPSs in combination with other PTPSs, such as a ubiquitin sequence. One such motif, the PEST sequence, is found extensively in short-lived proteins (Rogers et al Science. 234(4774):364-8 1986). A PEST sequence is a peptide sequence that is rich in proline (P), glutamic acid (E), serine (S), and threonine (T). This sequence is associated with proteins that have a short intracellular half-life. For example, Mouse Ornithine DeCarboxylase (MODC) is one of the shortest half-lived proteins in mammals due to the inclusion of PEST sequences in its carboxy terminus (Loetscher et al. J Biol. Chem. 1991 Jun. 15; 266(17):11213-20; Ghoda et al Mol Cell Biol. 1992 ay; 12(5):2178-85). Proteomics). Predicted PEST sequences have been identified in well-known antigens including E1A, c-myc, c-Fos, etc., (see, e.g., Rogers S et al. Science. 1986; 234(4774):364-368 and WO2004066935) and, e.g., SEQ ID NO:534. Additional PEST sequences and related PMCs are described in Doody K M et al. Cell Res. 2014; 24(9):1027-1028; Belizario J E et al. Curr Protein Pept Sci. 2008; 9(3):210⁻²²⁰; and Joshi S N et al. MAbs. 2012; 4(6):686-693. PEST motifs also can be considered “degrons,” discussed elsewhere.

In aspects, FPs can comprise ≥1 PTPS(s) that comprise sequences from or that are highly similar to one or more “ubiquitin like proteins.” An example of such a protein is FAT10. See, e.g., Mark Steffen et al. Molecular and Cellular Biology April 2005, 25 (9) 3483-3491.

In aspects, inclusion of one or more PTPSs in EPs detectably or significantly enhances proteasomal processing of the associated FP. In aspects, the inclusion of 1+ PTPSs in FPs also DOS enhances one or more aspects of immune response associated with the administration and expression of the construct, such as one or more MHC I or MHC II responses.

In aspects, PTPSs of FP(s) comprise, PC, GCO, or consist of ≥1 ubiquitin sequences. FPs can comprise any suitable number of ubiquitin sequences. In aspects, FP(s) comprise only a single ubiquitin sequence. In aspects, an FP comprises only one PTPS. In one aspect a fusion protein comprises only one PTPS and that one PTPS is a ubiquitin (Ub) sequence. In aspects, PTPS(s) comprise polyubiquitin (polyUb) sequence(s).

In aspects, the PTPS comprises a Ub sequence that comprises both a recognition element and a Ub attachment site. In aspects, EPs include FPs comprising four or more Ubs, such as five or more Ubs, six or more Ubs, or seven or more Ubs. In aspects, however, the invention alternatively provides fusion proteins comprising less than four full Ubs, but which still exhibit detectable or significant proteasomal targeting and other polyUb functionalities. Thus, in aspects, the invention provides constructs encoding FPs comprising three or more Ubs, and in another aspect the invention provides fusion proteins comprising less than 3 or 4 Ubs, such as 2-3 Ubs. In aspects, CEPESCs encode FPs comprising ≥1 at least partial Ubs that still impart polyUb functionality when combined with other Ubs. E.g., FPs can comprise 1, 2, or 3 partial Ubs in combination with 2, 3, 4, or 5 Ubs, such as 1 partial Ub in combination with 3 complete, generally complete, or essentially complete Ubs. In aspects, the inclusion of a polyUb sequence in FP(s) results in a DOS increase in MHC I-mediated activity with respect to one or more antigens in or AW the FP.

In aspects, one, most, generally all or all of the Ub sequences of a fusion protein are positioned at or near the N-terminus of the fusion protein (e.g., in the first 10%, first 20%, or first 25% of the amino acid sequence). In aspects, one or more Ub sequences are positioned in the middle of the fusion protein. In aspects, a fusion protein is provided that lacks any Ub sequences in the C-terminal ⅓^{rd}, \vec{1/4}^{th}, or ⅕^{th} of the fusion protein, or lacks any Ub sequence at the very C-terminus of the fusion protein. However, in aspects a fusion protein will comprise one or more Ub sequences in such a C-terminal portion of the fusion protein. In aspects, such C-terminal Ub sequences are metabolically stable and not cleaved from the fusion protein. In aspects, such metabolically stable Ub sequences are still able to detectably if not significantly induce proteasomal degradation of some, most, generally all, or all of the amino acid sequence(s) associated with the Ub sequence.

In aspects, the PTPS, such as Ub(s) exhibit the ability to DOS enhance CD8+ responses against antigen(s) associated with the Ub in a fusion protein, such as a Ub: gD-antigen fusion protein, Ub: gD-antigen: gD fusion protein, or a Ub: gD-antigen: Ub: antigen: gD-antigen fusion protein. In aspects, such a fusion protein comprises one or more subdominant epitopes or a composition comprises a separate nucleotide sequence encoding a Ub: subdominant epitope fusion protein that is expressed independently but in association with expression of a gD-antigen fusion protein and the presence of the Ub detectably or significantly enhances one or more immune responses to the subdominant epitope, such as a CD8+ response.

In aspects, Ub sequence(s) is/are cleaved from the fusion protein. In aspects, the Ub sequence acts as a cleavage site, creating two polypeptides after expression and cleavage, the Ub-associated polypeptide and a second polypeptide. In this respect, some polypeptides comprising Ub sequences can act as multi-cistronic, e.g., bicistronic constructs, which are discussed in more detail above. Such applications are exemplified in US20150266945.

In one aspect the Ub contained in a fusion protein encoded by a construct of the invention is or comprises a K48 chain Ub. In one aspect such constructs and fusion proteins are associated with detectably or significantly enhanced levels of proteasomal targeting of the fusion protein, fusion protein degradation, MHC antigen presentation, or a combination of any or all thereof. In aspects, the fusion protein also comprises or consists of one or more K63-linked Ubs. In such aspects, the fusion protein or construct is detectably or significantly associated with detectably or significantly enhanced DNA repair, ribosomal biogenesis, degradation of the fusion protein, or a combination thereof. In aspects, the fusion protein comprises both a K63-linked Ub and a K48-linked Ub and the fusion protein is associated with a detectable enhancement in fusion protein degradation as compared to a fusion protein comprising either one type of Ub on its own.

In aspects, one or more Ub sequences contains several, e.g., ˜7, lysine residues that can serve as substrates for ubiquitination, enabling the generation of seven different inter-ubiquitin linkage types. In aspects, the Ub comprises an amino-terminal methionine (M1) that can act as a Ub acceptor site. In aspects, however, the Ub or polyUb of the fusion protein lacks an N-terminal Met residue. It will be understood that the discussion of the characteristics of Ub sequences herein can be applied to polyUb fusion proteins and, as such, the disclosure of any aspect with reference to a Ub sequence of any type or characteristic will be understood as providing implicit support for polyUb sequences comprising one or more Ubs of such type or having such characteristics. PolyUbs encoded by a fusion protein, formed by further ubiquitination of expressed fusion protein Ub sequences, or both, can exhibit any suitable PolyUb form. In aspects, the fusion protein encodes a linear polyUb form or a branched polyUb; forms a linear polyUb or branched polyUb, or both. Various forms of polyUb are discussed in, e.g., Rieser E et al. Trends Biochem Sci. 2013; 38(2):94-102.

In aspects, FPs comprising ≥1 Ubs that are modified to enhance stability of the Ub sequence and are associated with reduced cleavage of the Ub sequence. One example of such a Ub sequence is Ub-G76A, featuring a Gly76 to Ala76 mutation of the last residue of a typical Ub sequence. In aspects, G76 is substituted with another non-polar amino acid residue, such as I, L, or V.

In aspects, the fusion protein comprises only PTPS or ERTPS targeting sequences. In aspects, the fusion protein lacks any lysosomal targeting sequences (e.g., SEQ ID NO:514). In other aspects, the fusion protein will comprise one or more lysosomal targeting sequences in addition to one or more PTPSs, and optionally also one or more ERTPSs.

Ub sequence(s) incorporated in a fusion protein can be of any suitable source or composition. In aspects, Ub sequence(s) are from the species that is the primary target for the CEPESC. Thus, for example, the Ub sequence(s) can be, e.g., pig, cow, horse, dog, cat, or human Ubs, functional fragments thereof, polyUbs that are a mixture thereof, and can comprise functional variants thereof which are, e.g., highly related or substantially identical to one or more naturally occurring Ub sequences or functional fragments thereof. In aspects, the Ub sequences are from a naturally occurring Ub, a functional fragment thereof, or both, as in the case of a polyUb comprising complete and partial naturally occurring Ubs.

A ubiquitin sequence can correspond to any type of naturally occurring Ub sequence or a functional fragment or variant thereof. In many organisms, ubiquitin is encoded by 4 different genes. For example, the UBA52 and RPS27A genes code for a single copy of ubiquitin fused to the ribosomal proteins L40 and S27a, respectively, whereas the UBB and UBC genes code for polyubiquitin precursors (polyUb and polyUC) with exact head to tail repeats, the number of repeats differ between species and strains.

In aspects, the fusion protein comprises at least one Ub that also is a Ub or Uc precursor sequence, such as a polyubiquitin B sequence (which comprises three direct repeats of a typical ubiquitin coding sequence with no spacer sequence). An example of such a ubiquitin B polyUb is SEQ ID NO:1, GenBank Accession No. AAA31133.1.

A Ub or polyUb can be placed in any suitable orientation in the fusion protein. In aspects, a Ub, such as a polyUb, is placed downstream of one gD sequence and upstream of a second gD sequence, such as a first gD sequence that is an N-terminal gD sequence fragment or variant and a second gD sequence that is a C-terminal gD fragment or variant or two partial or complete gD sequences that overlap in terms of structure/composition. In one such case, the Ub sequence can be placed indirectly or directly adjacent to one or more antigenic sequences that are also positioned between the gD sequences (e.g., a sequence according to the structure gD1-antigen-Ub-gD2). In another example, a fusion protein can comprise two Ub sequences, such as polyUb sequences, positioned between gD sequences with an intervening antigenic sequence (e.g., according to the structure gD1-antigen-Ub1-antigen-Ub2-gD2). In yet another aspect, the invention provides gD-antigen fusion proteins according to the formula Ub1-antigen(x)—Ub2-gD (antigen(x) signifying that the region can contain two or more antigens, such as 2-10, 3-12, 4-12, 3-9, 4-8, or 2-6 antigens). Another type of fusion protein comprises a sequence according to the formula gD1-Ub-antigen(x)1-Ub2-antigen(x)2-gD2. Still another fusion protein type comprises a sequence according to the formula Ub-antigen(x)-Ub2-antigen(x)2-gD (e.g., where the C-terminal gD primarily consists of or generally consists of a gD profusion domain). Still another example of a fusion protein of the invention is a trap protein, such as PDL1 trap (discussed further below) in combination with Ub, antigenic, and gD sequences, such as a sequence according to the formula PDL1trap-Ub1-antigen(x)-Ub2-gD (other trap proteins, such as a DEC-205 trap, could be used in place of the PDL1trap in such a fusion protein). In any of these fusion proteins, one, two, three, four, or more spacer sequences can be introduced between the Ub sequences and other sequences, between the antigenic sequences, or in any other suitable position.

In aspects, Ub sequences of FP(s) are modified so as to remove one, some, most, generally all, or all of any potential glycosylation sites in the Ub sequence, e.g., by introducing N-to-D substitution(s) in the Ub sequence, as described elsewhere with respect to certain antigenic sequence variants. Ubiquitin-fused genetic vaccines that enhance both antigen degradation and immune presentation adaptable to aspects are described in, e.g., M. A. Barry et al, Nature, 377 (1995), pp. 632-635; T. W. Tobery, et al. J. Exp. Med, 185 (1997), pp. 909-920; F. Rodriguez et al. J. Virol, 71 (1997), pp. 8497-8503; G. Delogu et al. Infect. Immun, 68 (2000), US 20090221682; US20040170643; U.S. Pat. No. 9,079,966; Dutton J L, et al. PLoS One. 2013; 8 (10): e76407; and Chen J H, et al. Hepat Mon. 2011; 11(8):620-628.

In aspects, FPs comprise one or more ubiquitin-like (UbL or UBL) sequences, which either correspond to naturally occurring ubiquitin-like proteins, comprise functional fragments of such proteins, or comprise a functional variant of such a protein or fragment, such as a variant that is very related or substantially identical to such a wild-type UbL protein (UBLP). In this disclosure a UBLP means any member of the UBL family of proteins that is not a ubiquitin. Collectively, ubiquitin and ubiquitin-like proteins are referred to as “ubiquitons.” In aspects, FPs comprise ≥1 ubiquiton(s). In aspects, FPs comprise UBL sequence(s) capable of conjugation (sometimes known as Type I UBLs). Examples of UBLs that exhibit such properties include SUMO, NEDD8, ATG8, ATG12, URM1, UFM1, FAT10, and ISG15 UBLs. In aspects, the fusion protein comprises a UBL that does not exhibit covalent conjugation (a Type II UBL). Another example of a UBLP is a Rad23 protein (e.g., human Rad23b) (Goh A M et al. BMC Biochem. 2008; 9:4. Published 2008 Jan. 30, and Liang R Y et al. J Mol Biol. 2014; 426(24):4049-4060. Still another example of a UBL is NEDD8 and its counterpart NUB1. Sequences according to the formula A(X4)L(X10)L(X3)L are conserved in these NEDD8-binding sites among human and other mammals and can be incorporated into FPs to promote proteasomal processing/targeting. SFE Tanaka T et al. J Biol Chem. 2003; 278(35):32905-32913.

In aspects, the fusion protein comprises a UBL sequence that can also be characterized as a functional sequence of a ubiquitin-like modifier (ULM) UBLP or a functional variant thereof. In aspects, FP(s) comprise a functional ubiquitin-like domain protein (UDP) or a functional variant thereof.

In aspects, FP(s) also can comprise degron(s), which, in aspects, DOS improve targeting of the associated FP to the proteasome, processing of the fusion protein by the proteasome, or both. In aspects, FP(s) also comprise one or more UBL sequences or ubiquitin sequences. In cases, degron(s) DOS enhance degradation of an FP or part of an FP (e.g., a cleaved part). In the case of proteasomal degradation, a specific degron might initiate polyubiquitylation by an E3 ligase or might target the substrate directly to the proteasome or both. In aspects, degrons are ˜6 to 10 AARs. In aspects, some, most, or all degron(s) are located within relatively flexible regions of an FP (e.g., located within 20 AARs of a linker), or a cleaved part, enabling interaction with other proteins (e.g., ubiquitin, E3 ubiquitin ligase, etc.). In aspects, FPs comprise degron(s) that comprise one or more Lys or Arg residues that are structurally exposed. In aspects, FP(s) comprise one or more degrons that are “Ubiquitin-dependent.” In aspects, FP(s) also comprises one or more degrons that can be characterized as “Ubiquitin-independent.” In the first case intracellular protein degradation is mediated largely by the ubiquitin (Ub)-proteasome system (UPS) and in the second case by autophagy-lysosome pathways, with molecular chaperones being a part of both systems. In aspects, FP(s) comprise an N-degron, a C-degron, or a combination thereof. In aspects, FP(s) comprise inherent degron(s) (e.g., the destruction box of cyclins). In aspects, FP(s) comprise acquired degrons (e.g., phosphorylated AARSs that act as degrons). In aspects, FPs comprise one or more D box degrons (e.g., sequences according to the formula R[KR][AP]Lx[DE][ILV][TS]N) (e.g., SEQ ID NOs: 535-567), KEN box degrons (e.g., that contain a GxEN motif, such as SEQ ID NOs:568-589), ABBA degrons (comprising a structure according to the formula [ILMVP][FHY]x[DE], [FILV]x[ILMVP][FHY]x[DE], or [KR]xx[ILV][FHY]x[DE], e.g., SEQ ID NOs:590-594, or a combination thereof. Other types and examples of degrons suitable for inclusion in FPs include, e.g., LLRL tail degrons, IR tail degrons, etc., such as SEQ ID NOs:595-597 and the Cop1-binding Trib peptide (WO2019118893).

FPs can also comprise other sequences that target FPs for ubiquitination. Identification of ubiquitin sites that can be adapted to FP(s) is is discussed in, e.g., Nguyen, V et al. BMC Bioinformatics 16, S1 (2015). In aspects, FPs comprise, or compositions comprise NAMs encoding, an E3 ligase ligand, which can promote or enhance polyubiquitination or a proteolysis-targeting chimeras (PROTACs) (SFE Qi J, Zhang G. Future Med Chem. 2019; 11(7):723-741, for a discussion of PROTACs). Other Ub-targeting sequences that may be adapted to FPs/compositions are described in Adams, Trends Mol Med. 2002; 8(4 Suppl): S49-54). In aspects FPs comprise an E3 ligase e.g., CHIP (carboxyl terminus of Hsc70-interacting protein), ≥1 cytosolic chaperons (e.g., Hsp70/Hsp90), etc.

In aspects, FPs comprise degradon(s) (WO2017079723 and US20050152888). A degradon is a sequence comprising a target binding domain and a proteasome-binding domain (e.g., a Ub or ULP domain). Optionally, degradon(s) can comprise a linker sequence positioned between the degradon target binding domain and proteasome binding domain.

In aspects, degrons or related elements are contained in FPs, optionally including other elements (e.g., Ags, gDS(s), etc.). Examples of fusion protein sequences comprising both degrons and Ubs/UBLP sequences are described in, e.g., Inobe T, et al. Biochem Biophys Res Commun. 2018; 501(4):948-954 and US 20180327462. PEST degron FPs comprising Ub AARSs are described in US20190322714.

In aspects, FPs/EPs comprise AARS(s)/PPT(s) that are heterologous to the referenced sequence(s) and target the endoplasmic reticulum (ER), promote processing of the polypeptide in the ER, or both. Such a heterologous sequence can be referred to as an ER-targeting/processing sequence or “ERTPS.” Such FP(s) can be, e.g., gDAgFPs or other FPs. In aspects, ERTPS(s) DOS increase targeting of the ER, MHCI presentation, or IR(s). ERTP(s) are described in, e.g., U.S. Pat. No. 5,846,540; EP2167123; Xu et al. Virology. 2005; 334(2):255-263; Sher et al, Am J Cancer Res. 2019; 9(9):2028-2036; Martinez-Puente et al. Cell Stress Chaperones. 2019; 24(1):149-158; and Wang et al. Eur J Immunol. 2004; 34(12):3582-3594.

In aspects, FP(s) comprise ERTPS(s) selected from, e.g., SEQ ID NOs: 511 and 598-642, SEQ ID NO:644, and SEQ ID NO:645. In aspects, FP(s) also include a KDEL sequence. In aspects, FP(s) also include tPA sequence(s), calreticulin sequence(s), chaperone Grp170 sequence(s), or an IgKappa-chain leader sequence, with ERTPS properties.

FP(s) also can include ER-associated protein degradation (ERAD)-associated factors. In other aspects, FPs lack any ERAD-associated factor-encoding sequences. In aspects, such FP(s) exhibit DOS degradation by the 26S proteasome. In aspects, such FP(s) also comprise polyUb sequences or a composition can comprise NS(s) encoding one or more ERAD factors separately from any PTPS-containing FPs (e.g., a ubiquitinated FP). Exemplary ERAD factors include BiP, an ER-luminal heat shock protein of 70 kDa (Hsp70) and ER-associated lectins (e.g., calnexin and calreticulin), Herp, Hrdl, and Derlin-1, and ubiquitin ligases such as gp78, TRC8, and Doa10/TEB4. Herp also is a UBLP, reflecting, possible overlapping functionality of factors (degrons also can promote ERAD). gD sequences also can promote ER targeting/processing. However, while there can be overlap between ERAD factors and PTPS-related factors or elements, such elements, e.g., ERTPSs and PTPSs typically are distinct in constructs. In aspects, the inclusion of one or more ERTPSs detectably or significantly enhances ER processing. In aspects, ERTPSs DOS enhance one or more aspects of IR(s), such as MHC I IR(s) or MHC II IR(s).

In aspects, a composition is provided that comprises one or more NAMs encoding at least two separate/distinguishable fusion proteins, each of the at least two fusion proteins comprising one or more antigens, and the first of the at least two fusion proteins comprising one or more PTPSs (and optionally lacking any ERTPS) and the second comprising an ERTPS (and optionally lacking any PTPSs). In aspects, the ≥2 fusion proteins are encoded by distinct sequences contained in different NAMs of a composition or a method is employed in which such different NAMS are associatively administered, such as through co-administration or sequential administration. In aspects, CEPs, such as FP(s) comprise ETS(s). An ETS is an AARS that DOS binds to a target presented outside of cells of expression (COEs) (e.g., on another cell or otherwise in the milieu/microenvironment). An RBD is a typical type of ETS, but other types of ETSs also can be present. EPs can comprise any suitable type of ETS and any suitable number of ETSs. Typically, a single PPT will comprise a single ETS. However, in aspects, a single PPT can be bi-specific, comprising 2 ETSs. Single PPTs can also comprise additional ETSs (e.g., a WT gDP typical targets 3 different receptors). Multimeric PPTs often are multi-specific. E.g., Ab PPTs are often bispecific and other multimeric PPTs can be specific for 3, 4, or ≥5 ETSs.

In aspects, constructs encode FP(s) comprising sequence(s) that bind to receptor(s) on immune system cell(s) (“immune cell targeting sequences” or “ICTSs”). Effectiveness of binding of an ICTS to an immune cell receptor (ICR) means DOS level of binding or binding associated with DOS IC receptor binding-associated cellular/physiological event(s), such as uptake of the FP, inducement of a receptor binding-mediated effect(s), etc.

In aspects, an ICTS binds ≥2 receptors (e.g., gDSs can bind ≥2 IC receptors (including HVEM & Nectin-1 regarding HSV-1 gD and Nectin-1 and Nectin-2 regarding HSV-2 gD). In aspects, an ICTS also can bind homolog(s) of receptor(s) (e.g., as PRV gD is expected to bind both human Nectin-1 as well as its porcine homolog). In aspects, ICTS(s) of a FP preferentially bind or specifically bind target(s) (as described elsewhere).

In aspects, an EP comprises ≥1 ETS. In aspects, an EP comprises only 1 ETS. In aspects an ETS is positioned at a terminus of the EP or near such an AARS terminus (e.g., within 20%, 15%, or 10% of the terminus based on number of AAs). In aspects, ETSs are positioned on or near both termini of an EP. In aspects, 1 ETS is positioned at or near an EP terminus. In aspects, the ETS is an RBD (e.g., a WT RBD, an FF, or a FV).

In aspects, the target immune cell(s) (IC(s)) that comprise target receptor(s) bound by TS(s)/ICTS(s) of FP(s) are APC(s). An “antigen-presenting cell” or “APC” typically can process and display foreign antigens in association with major histocompatibility complex (MHC) molecule(s). An APC targeted by an extracellular TS can be any suitable APC. Examples of such cells include dendritic cells (DCs), macrophages, Langerhans cells, and B cells. Other cells that can act as APCs include, e.g., γδ-T cells, NK cells and fibrocytes (non-conventional APC(s)). In aspects, a fusion protein is expressed from a construct of the invention that comprises a TS for a non-conventional APC. In one example, the invention provides a fusion protein that comprises a TS that binds to one or more targets expressed on γδ-T cells, such as a porcine γδ-T cell, e.g., in a fusion protein comprising one or more ASFV antigens.

In aspects, a target cell can be characterized as an adaptive IC, i.e., a B cell or a T cell). In aspects, the target cell also can be characterized as a cell of the innate immune system, such as a macrophage or eosinophil. In still another aspect, the target cell is an ITIC, e.g., a DC or an NKC. A target cell may meet two or more of the categories that define various facets of such aspects, as in the case of DCs.

In aspects, FP(s) comprise ICTS(s) that target a receptor on dendritic cell(s) (DC(s)). In aspects, FP(s) comprise gDS(s) or other ITCS(s) that effectively bind(s) a dendritic cell receptor (DCR). In aspects, constructs of the invention encode at least one dendritic cell receptor binding sequence/ligand (a DCR targeting sequence or “DCRT”) that is heterologous to naturally occurring gD proteins (and that typically exhibits ≤˜40% identity, ≤˜30% identity, or 9-20% identity to one or more wild-type gD proteins/sequences, such as all naturally occurring gD proteins). In aspects, FP(s) comprise a combination of DCR-binding gD sequences (gDS(s)) and one or more DCR-binding heterologous sequences. In aspects, the only DCR-binding sequences of FP(s) are gD sequence(s). In aspects, the only DCR-binding sequences of a fusion protein are non-gD sequence(s) (in some such aspects the fusion protein comprises non-DCR-binding gD sequences and other polypeptides encoded by constructs of the invention comprising non-gD DCR-binding sequences lack any gD sequences).

In aspects, FP(s) comprise DCRTS(s) that is/are a sequence of an antibody that effectively binds to a DCR or an FF/FV that also at least suitably binds the DCR. In aspects, FP(s) also comprise(s) a DCRT that comprises a sequence from a naturally occurring non-Ab ligand for a DCRT or a sequence that is at least related thereto and that effectively binds the DCR. In aspects, FP(s) lack any anti-DCR antibody sequences. In aspects, FP(s) lack any antibody sequences or any sequences that are highly related or substantially identical to Ab sequence(s). “Lacking” a sequence in this and other respects means that a sequence identical to such a sequence is not present, but any statement in this disclosure will be understood to implicitly also provide support for/disclose lacking sequences that are at least highly related to the referenced missing sequence. In aspects, a DCRTS FP comprise(s) synthetic non-antibody DCR-binding sequence(s). In aspects, the DCRTS is a multimeric protein, a multivalent protein, or both. One example of a multimeric synthetic DCRTS protein that can be adapted for use in contexts of this invention are the short (12-mer and 6-mer), Lys core trimeric CLEC10A-binding proteins described in Eggink L L et al. J Immunother Cancer. 2018; 6(1):28, adaptable to FP(s). In aspects, FP(s) lack any sequences of more than 25 AAs in 1, 2, or 3+ contiguous sequences that collectively exhibit more than about 80% identity, ≥90% identity, ≥95% identity, or ≥97% (e.g., 98% or 99%) identity to target-binding sequences of any Ab against receptors discussed herein.

DCRTs in FPs can target any suitable DCR(s). In aspects, targeted DCRs include Langerin/CD207 (SFE Juliana Idoyaga et al. PNAS. February 2009, 106 (5) 1524-1529; and Idoyaga J et al. J Immunol. 2008; 180(6):3647-3650); mannose receptor (MR); Toll-like receptors (TLRs); C-lectin receptors (CLRs) and other pattern recognition receptors (PRRs) (SFE Lundberg K et al. Immunology. 2014; 142(2):279-288; van Kooyk Y. Biochem Soc Trans. 2008; 36 (Pt 6):1478-1481; and van Dinther D et al. J Leukoc Biol. 2017; 102(4):1017-1034), such as DC-SIGN (DC-specific ICAM-3-grabbing nonintegrin) (SFE Jarvis C M et al. PNAS 2019; 116(30):14862-14867; Engering A et al. JImmunol. 2002; 168(5):2118-2126; Zhou T et al. Cell Mol Immunol. 2006; 3(4):279-283; Noll A J et al. Biochem J. 2016; 473(10):1343-1353; Feng D et al. Clin Exp Immunol. 2018; 191(1):107-115; Cai M et al. J Transl Med. 2013; 11:103; and US20080019998), C-type lectin 12a (Clec12a), C-type lectin immunoreceptor (CIRE), DC-associated C-type lectin 1 (Dectin 1); Heat Shock Protein receptors; CD11c, and other DC-associated integrin receptors; FC receptors (e.g., FC gamma receptor (SFE Guilliams M et al. Nat Rev Immunol. 2014 May; 14(5):349 and 14(2):94-108)); the CD40 receptor (SFE Ma D Y et al. Semin Immunol. 2009; 21(5):265-272; CLEC9A (SFE Zhang J G, et al, Immunity. 2012; 36(4):646-657); chemokine receptors e.g., CCR7, CCR4, CCR6, and CXCR5 (SFE Oppenheim J J et al. Arthritis Res. 2002; 4 Suppl 3(Suppl 3):S183-S188); CLEC10A; MRC1, Dectin-2 (CLEC6A); Mincle; MMR; DNGR-1; DC-ASPGR; LOX-1; BST-2; DCIR (CLEC4A, Clec4A2); f4/80-like receptor (FIRE); DCIR2 (CLEC4A4); scavenger receptor (SR); DCAR1; and dendritic and epithelial cell receptor of 205 kDa (“DEC205” or “DEC-205”) (another type of CLR), etc. (Nchinda G et al. J Clin Invest. 2008; 118(4):1427-1436; Sehgal K et al. Immunol Lett. 2014; 162(1 Pt A):59-67; Hossain M K, Wall K A. Cancers (Basel). 2019; 11(3):418; and Hoober J K et al. Front Immunol. 2019; 10:2880). In aspects, the DCRTS(s) of FP(s) comprise FF(s) of WT DCR ligand(s). Naturally occurring peptide ligands of DCRs include Flt3L, F-actin, myosin II, exposed actin filament proteins, defensins, and chemokines such as SLC/CCL21, ELC/CCL19, and BLC/CXCL13. In aspects, FP(s) also comprise DCRTs that are antigens known to bind DCRs, such as HCV core protein, HIV envelope protein, and the H. pylori Lewis Antigen. In aspects, FP(s) comprise, MC, or only comprise DCRTS(s) that bind(s) stimulatory DCR(s). In aspects, such FP(s) comprises no gD sequences. In aspects, such FP(s) comprises a gD-antigen portion, such as an antigen-gD profusion domain fusion protein. In either case, such a FP(s) also can comprise a gD signal sequence.

In aspects, FP(s) comprise DCRTS(s) that bind(s) DCR(s) on myeloid DCs (MDCs) or DCs that are characterized generally as conventional DCs. In cases, MDC(s) targeted by a DCRTS can comprise BDCA1+ MDCs, BDCA3+ MDCs, or both. In aspects, FP(s) comprise DCRT(s) that also binds to a DCR on plasmacytoid dendritic cells (PDCs) (e.g., BDCA-2, DCIR, TLR-7, or TLR-9). In aspects, FP(s) comprise DCRTS bind(s) to a DCR on CD8α−/CD1c+ DCs (e.g., DEC-205, DCIR-2, or TLR-7). In aspects, DCTRS(s) also bind(s) DCR(s) on CD8α+/CD141-DCs (e.g., CLEC9A, DNGR-1, TLR-3, DEC-205, and DCAR1). In aspects, FP(s) comprise DCRTS(s) that binds both such types of cells (e.g., a DCTRS that binds DEC-205). In aspects, DCTRS(s) in FP(s) also bind(s) to a DCR expressed in skin DCs (e.g., DC-SIGN or Langerin). In aspects, DCRTS(s) also binds a receptor on DC precursor cells. In aspects, DCRTS(s) binds to a receptor on mature DCs. In aspects, DCRTS(s) binds a receptor expressed in mature DCs. See generally Hossain M K et al. Cancers 2019; 11(3):418 and Macri C et al. Semin Cell Dev Biol. 2018; 84:11-21. In aspects, DCRTS(s) bind any combination of these or other DCR(s).

In aspects, FP(s) comprise a variable region of an anti-DCR antibody, an effective single-chain variable fragment (ScFv), or an effective combination of anti-DCR CDR regions. Examples of ScFv molecules that target DCRs are described in, e.g., WO 2019/082208.

In aspects, FPs comprising DCRTSs are multimeric. In aspects, the multimeric DCRTS fusion protein comprises an antibody multimerization domain. In aspects, the DCRTS fusion protein is a trap protein formed of a multimerization domain and two or more receptor-binding ligand domains.

In aspects, DCRTS(s) DOS enhance internalization of Ags by MHC class I/II molecules, IR(s), or both. In aspects, DCRTS(s) result(s) in enhanced Th1 response, enhanced CD8+ response, or both (against DCA(s)). In aspects, the presence of DCRTS(s) results in a DOS enhancement of both CD4+ and CD8+ T-cell responses. In aspects, DCRTS(s) also DOS promote(s) cross-presentation (i.e., presentation of exogenous antigen by MHC class I). In aspects, DCRTS FPs induce cross-presentation of AgFPs or co-expressed Ags. In aspects FP(s) comprise a DCRTS(s) that targets Langerin on Langerhans cells, a TLR, a DC-associated heat shock protein (e.g., by including an HSP-binding lectin-like oxidized LDL receptor 1 sequence), DC-SIGN, or DEC-205. In aspects, a FP comprising a DCRTS is expressed in the context of cells that are activated by an initial exposure to an ITICSTAP, such as EAT-2. An aspect provides a method of inducing IR(s) comprising activating DCs by, e.g., expression of an ITII-encoding nucleotide sequence or administration of an ITII, such as an EAT-2, followed delivery of DCRTS FP(s).

In aspects, FP(s) comprise DCRT(s) that bind(s) to a DEC-205 protein (e.g., human DEC-205, porcine-DEC-205, chicken DEC-205, or homolog(s) thereof) (SFE Staines K et al. PLoS One. 2013; 8(1): e51799).

In aspects, a DEC-205-binding FP is co-administered with a construct comprising ES(s) encoding one or more DC stimulating factors, such as an ITICSTAP, e.g., an EAT-2 PPT. In aspects, a DEC-205-binding FP is administered following initial DC stimulation, such as DC stimulation with an EAT-2 polypeptide or other ITIC IM. In aspects, a DEC-205-binding FP is administered with agent(s) that detectably enhance the acidity of the DC microenvironment, detectably improve DEC-205-uptake of the fusion protein or DEC-205-related biological activity or exhibits a combination thereof.

In aspects, DEC-205-binding sequence(s) or other DCRTS(s) in EP(s) results in DOS enhanced DC binding or DC uptake of the fusion protein (e.g., in one aspect the presence of the DCRTS, such as a DEC-205 TS, results in an increase in DC uptake of the associated fusion protein of at least about 20%, ≥50%, ≥100% (2×), or ≥200% (3×)). In aspects, the delivery of an effective amount of the DCRTS fusion protein results in a detectable or significant increase in the amount of interferon gamma-secreting T cells in the subject receiving the DCRTS FP. In aspects, a DEC-205-targeting sequence keratin sequence(s) (e.g., a sequence of or VR, HR, or SI to the sequence of a C-Terminal Gly-Rich Region of keratin 1 and keratin 10). In aspects, a keratin/kerain-like DEC-205 TS comprise(s) glycine- and serine-rich domains in a X(Y)ₙ format, where X represents an aromatic or a long-chain aliphatic residue and Y is Gly or Ser (in aspects the X(Y)ₙ pattern in such domains is quasi-repetitive, forms Gly loops, or both). Keratin sequences of FP(s) can be exposed, e.g., by design of the fusion protein (e.g., by connection to a flexible linker). Relevant PMCs are described in Cao L et al PNAS 2016; 113(47):13438-13443.

FP(s) also can comprise keratin PPT(s)/AARS(s) that exhibit other functions than DEC-205 binding. E.g., FP(s) can comprise kertain-4, keratin-6, keratin-8, keratin-13, keratin-17, kertain-18, keratin-19, etc., or sequences of other PPTs known to induce immune cell responses, e.g., IC cytopathic activities, related to pathogenic infection, cancer, or both. Keratin AARS(s) can act as, e.g., “eat me” signals to IC(s), e.g., innate immunity cells or innate trained immunity cells. In aspects, expression of such keratins can add structural and functional support to cells to resist physical degradation/attack by disease-causing agents, such as pathogenic microbes. Related compositions and principles are described in, e.g., Geisler F, et al. Cells. 2016; 5(3):29.

In aspects, FP(s) comprise anti-DEC-205 antibobdy DEC-205 TS(s). Anti-DEC-205 antibodies (Abs) include, e.g., the Ab component of CDX1401 (Celldex Therapeutics, Hampton, NJ, USA) (which also comprises NY-ESO-1 and TLR agonists resiquimod (TLR 7/8 agonist) and Hiltonol (polyICLC, TLR3 agonist) (Sehgal K et al. Immunol Lett. 2014; 162(1 Pt A):59-67). In aspects, FP(s) comprise anti-DEC205 single-chain Fv fragment(s). Additional anti-DEC-205 Abs or PMCs are known (see, e.g., Hua Y et al. Int Immunopharmacol. 2017; 46:62-69; Niezold T et al. Immunology. 2015; 145(4):519-533; Birkholz K et al. Blood. 2010; 116(13):2277-2285); & US20130101593.

In aspects, delivering an EA of DEC-205-binding FP(s) causes a DOS increase in relevant MHC II products, antigen presentation, or both. In aspects, antigen presentation associated with DEC-205-binding FP(s) is increased by at least about 25%, ≥˜33%, ≥˜50%, ˜100% (2×), ≥˜200% (3×), ≥˜5×, ˜-10×, ≥˜20×, ≥˜50×, or even at least about 100×. In aspects, the administration of the DEC-205-TS FP results in DOS enhanced IL-2 expression (e.g., from CD4+ T cells (e.g., expression levels of at least about 0.2, 0.3, 0.4, 0.5, 1, or 1.2 ng/mL)). In aspects, administration of EC-205-TS FP(s) also results in DOS enhancement of TNFα expression in subject(s) or cells (e.g., expression levels of ≥˜0.1 or ≥˜0.2 ng/mL). In cases, administration of DEC-205-TS FP(s) also results in DOS enhancement of interferon gamma (IFNg) expression in subject(s) or immune cells (e.g., expression levels of at least about 0.3, 0.4, 0.5, or 0.7 ng/mL). In aspects, administration of DEC-205-TS FP(s) also results in at least about 2×, ≥˜3×, or 4×, 5×, 10×, or more expression of IL-2, TNFα, interferon gamma, or a combination of some or all thereof in the subject or cells. In aspects, delivering DEC-205-TS FP(s) results in DOS reduction of the IL-10/IL-2 ratio in a subject or cells. In aspects, delivery of DEC-205-binding FP(s) does not significantly alter the phenotype or functionality of DCs in the subject/milieu. Methods related to such aspects are described in, e.g., Birkholz K et al. Blood. 2010; 116(13):2277-2285. In aspects, delivery of DEC-205-binding FP(s) also does not DOS induce tolerance to any associated Ags expressed with or contained in FP(s).

Any aspect described here with respect to a subject, animal, vertebrate, cell, or cells, implicitly simultaneously provides support for related method, observance, etc., in related population(s) (e.g., in a statistically significant proportion of the referenced population of subject(s) in an adequately powered and properly conducted clinical trial).

In aspects, a DEC-205-binding FP is a multimeric protein, a multivalent PPT, or both. In aspects, DEC-205-binding FP(s) comprise extracellular target TS(s) (e.g., a gD receptor-binding gD sequence). In aspects, DEC-205-binding FP(s) is/are monospecific. In aspects, DEC-205-binding FP(s) is/are multi-specific (e.g., binding both Nectin-1 and DEC-205). In aspects, DEC-205-binding FP(s)/PPT(s) is/are DEC-205-trap PPT(s). In aspects, DEC-205-binding trap protein(s) comprise ≥1, 2, 3, 4, 5, 8 or more antigenic AARS(s), wherein the antigen sequences are optionally bound to each other, the DEC-205 TS, or both, by linker(s), and the Ags are also associated with internal TS(s), e.g., PTPS(s), ERTPS(s), or both. In aspects, DEC-205 trap PPT(s) is/are delivered with an ITII that DOS activates DC(s), e.g., an EAT-2 PPT, delivered before or expressed with DEC-205-binding trap PPT EPES(s).

In aspects, EP(s) comprise cell penetrating peptide sequence(s) (CPPS(s)). In aspects, CPPSs in EP(s) DOS facilitate cellular intake and uptake of EP(s) associated with the CPPS. In aspects, a FP(s) comprises a CPPS associated via a covalent bond. In aspects, an EP also comprises a CPP/CPPS bound to another EP via a non-covalent interaction. In aspects, CPPS(s) is/are a polycationic CPPS, amphipathic CPPS, hydrophobic/apolar, etc. Exemplary CPPS(s) include transactivating transcriptional activator (TAT) of an HIV (e.g., HIV-1) (e.g., RKKRRQRRRR (SEQ ID NO:729)), antennapedia, transportan, polyarginine, penetratin or any of the other CPPSs described in CYTOPLASMIC VESICLES-ADVANCES IN RESEARCH AND APPLICATION: 2013 Edition (Q Ashton Acton, General Ed.) (ISBN: 978-1-481-69922-8) (e.g., pgs. 68-69). In aspects, a CCPS-associated EP(s) have/has a size of ˜30 kDa˜150 KDa (e.g., about 30-120 kDa, ˜30-100 kDa, or ˜30-80 kDa). Additional CPPSs and aspects thereof that can be adapted to such aspects are described in, e.g., Ziegler A et al. Biochemistry. 2005; 44(1):138-148; Milletti F (August 2012). Drug Discovery Today. 17 (15-16): 850-60; & Stewart K M et al. Organic & Biomolecular Chemistry. 6 (13): 2242-55. In aspects, an EP comprises ≥1 CPPSs. In aspects, an EP comprises only 1 CPPS. In aspects, a CPPS is placed at a terminus or in the first/last 20% of an AARS of a FP. In aspects, a CPP is placed on the opposite end of a FP from an RBD. In aspects, the CPPS is indirectly bound to the rest of the FP, e.g., through a FL, MSL, or MSFL.

Constructs can comprise ≥1 antigen-encoding sequences (AgESs), expressed as antigens (Ags) and inducing IR(s) in TR(s) and cells.

In aspects, NAMs may be targeted to/taken up by APCs, such that AgESs are expressed in APCs and directly presented to lymphocytes. In aspects, NAM(s) is/are targeted to DOS promote uptake by APCs. E.g., association with some transfection-promoting agents, such as calcium phosphate nanoparticle(s) (CaPNP(s)), can DOS promote uptake of NAM(s) by APCs. Construct also can be contained in a vector that comprises APC targeting element(s) (e.g., an adenoviral vector comprising DCR-TS(s)).

In aspects, constructs comprising AgESs also are taken up by other cells of the subject/milieu (e.g., fibroblasts, epithelial cells, carcinoma cells, neurons, other immune cells (e.g., T cells or B cells), etc.). An EA of an expressed FP typically ss thereafter released from COEs and taken up by APCs in the subject or microenvironment/milieu. In aspects, FP(s) can comprise one or more APC targeting sequences, such as a DCRTS.

In aspects, Ag EP(s) are bound to major histocompatibility complex (MHC) class I and II molecules and brought to the surface of APC(s) associated with such MHCI and MHCII molecule(s). Ag(s) can be presented to cluster of differentiation 8 transmembrane glycoprotein (CD8+ T cells) or CD4+ cells. In aspects, Ag EP(s) DOS induce, i.a., immature CD8+ T cells to develop into mature antigen specific CD8+ T cells (aka, cytolytic T cells or killer T cells).

CEPESCs can encode any suitable number of Ag(s), each being any suitable type of Ag. The terms “antigen” and “antigenic” are known in the art and, accordingly, is not limited to any specific definition herein, other than expressed Ag(s) are peptides/protein(s). Ag(s) comprise epitope(s) (either linear, conformational, or both) associated with humoral (B-cell) and/or cellular (T-cell) antigen-specific IR(s). Ag(s) can comprise one epitope or multiple epitopes (e.g., FP(s) can comprise one antigen or ≥2 Ags). Generally, the term epitope also not intended to be limited herein. Epitopes bound by antibodies or B cells are referred to as “B cell epitopes” and the epitopes bound by T cells are referred to as “T cell epitopes.” Epitopes for NK cells also have recently been identified (SFE Sim et al. Human NK cell receptor KIR2DS4 detects a conserved bacterial epitope presented by HLA-C. Proc Natl Acad Sci USA. 2019; 116(26):12964-12973). In aspects, NSs encode at least one NK cell antigen, optionally in addition to T cell antigen(s) (e.g., at least one MHC-I antigen and at least one MHC-II antigen), B cell antigen(s), etc.

Antigens can be associated with any type of disease-causing agent (DCA), e.g., a tumor or a pathogen. Examples of Ags include Ag(s) that induce IR(s) against (and typically are from or related/similar to sequence(s) of) AChR (fetal acetylcholine receptor), ADGRE2, AFP (alpha fetoprotein), BAFF-R, BCMA, CAIX (carbonic anhydrase IX), CCR1, CCR4, CEA (carcinoembryonic antigen), EGP-2 (epithelial glycoprotein-2), EGP-40 (epithelial glycoprotein-40), EGFR(HER1), EGFR-VIII, EpCAM (epithelial cell adhesion molecule), EphA2, ERBB2 (HER2, human epidermal growth factor receptor 2), ERBB3, ERBB4, FBP (folate-binding protein), Flt3 receptor, folate receptor-a, GD2 (ganglioside G2), GD3 (ganglioside G3), GPC3 (glypican-3), GPI00, hTERT (human telomerase reverse transcriptase), kappa-light chain, KDR (kinase insert domain receptor), LeY (Lewis Y), L1CAM (LI cell adhesion molecule), LILRB2 (leukocyte immunoglobulin like receptor B2), MARTI, MAGE-A1 (melanoma associated antigen A1), MAGE-A3, MSLN (mesothelin), MUC16 (mucin 16), MUCI (mucin I), KG2D ligands, NY-ESO-1 (cancer-testis antigen), PRI (proteinase 3), TRBCI, TRBC2, TFM-3, TACI, tyrosinase, survivin, hTERT, oncofetal antigen (h5T4), p53, PSCA (prostate stem cell antigen), PSMA (prostate-specific membrane antigen), hRORl, TAG-72 (tumor-associated glycoprotein 72), WT-1 (Wilms tumor protein), and Ags of hepatitis B, hepatitis C, CMV (cytomegalovirus), EBV (Epstein-Barr virus), and HPV (human papilloma virus). In aspects, OSMGASAOA Ag(s) in EP(s) exhibit specific binding affinity for Ag-binding immune cell (IC) PPTs, e.g., TCRs, BCRs, MHC PPTs, etc.

In aspects, constructs encode EP(s) (being or including, e.g., FP(s) that comprise(s) non-conformational/linear epitope(s). In aspects, SMGASAOA epitope(s) in a FP or in CEP are linear. In aspects, methods or compositions also include conformational epitope(s).

The terms MHC Class I and MHC Class II herein are meant to apply to such molecules from any species and functional variants thereof, rather than indicating such sequences of any species. In this respect, MHC Class I molecules/antigens encompass HLA-A molecules/antigens and MHC Class II molecules/antigens encompass HLA-B molecules/antigens.

In aspects, Ag(s) comprise flanking sequences in addition to a core epitope sequence(s) (e.g., core sequences being 3-24, typically 7-10 amino acids long). Flanking sequences are typically identical to or highly related to the sequences of the protein from which core epitope sequence(s) is/are also derived. Flanking sequence(s) can be at least about 5, ≥10, ≥15, ≥20, ≥25, ≥30, or ≥40 amino acids in length upstream or downstream of the core epitope sequence(s). In aspects, Ag flanking sequence(s) DOS enhance MHC response(s) to the antigen.

Typically, Ag(s) is/are derived from a disease-causing agent (DCA), such as a pathogenic organism, virus, or a cancer cell. In aspects, an antigen also can be a variant of a DCA antigen (e.g., an editope, mimotope, etc.). Uncontradicted, aspects describing regarding one type of Ag/epitope implicitly provide support any other type of epitope/Ag described here. PMCs relevant to mimitopes are provided in, e.g., Zhao L et al. Expert Rev Vaccines. 2008; 7(10):1547-1555; Kozbor D. Immunol Res. 2010; 46(1-3):23-31; Chen L. Curr Opin Immunol. 1999; 11(2):219-222; and US20080019992 (with respect to cancer antigen mimotopes) and Zhong Y et al. Virol J. 2011; 8:542; Magliani W et al. Curr Med Chem. 2004; 11(13):1793-1800; U.S. Pat. No. 7,892,557, US20120148594, US20190070248, & WO2007022557A1 (regarding pathogen-associated mimotopes). See also Li D et al. Int J Biol Sci. 2018; 14(4):461-470; Partidos CD. Curr Opin Mol Ther. 2000; 2(1):74-79; U.S. Pat. No. 7,166,694; EP0387276B1, and Kieber-Emmons T. Immunol Res. 1998; 17(1-2):95-108. In aspects, EP(s) comprise mimitope(s) of non-protein Ag(s) (e.g., lipid(s), carbohydrate(s), or a mixture) or of a modified protein (e.g., glycoprotein or lipoprotein).

Antigenic PPTs/AARSs are typically immunogenic (where conformational changes are required to present an epitope an antigenic sequence or polypeptide might situationally not be immunogenic). Immunogenic PPTs & AARSs thus include antigenic PPTs & AARSs, but also can include polypeptides and sequences that primarily upregulate immune system activity through other functions. Examples of such immunogenic PPTs and sequences include cytokines, such as interleukins (ILs) (e.g., IL-2), and innate adaptive immunomodulators, such as EAT-2 PPTs. Immunogens include non-peptidic molecules, such as ISNSs, beta glucans, and lipopolysaccharides.

An “immunological response” or “immune response” (“IR”) means any detectable response, of the immune system or constituent(s)/component(s) thereof. In aspects, EP(s) cause DOS humoral IR(s), cellular IR(s) (e.g., IR(s) comprising IR(s) of cytolytic T-cell(s) (CTL(s)), helper T cell(s), or both), an innate immune response (mediated by cells of the innate immune system, e.g., macrophages), an innate trained/adaptive immune response (mediated by innate trained immune cells, e.g., natural killer (NK) cells (or NKCs) and dendritic cells (DCs), or both), etc. In aspects, EP(s) cause a DOS increase in cytokine(s), chemokine(s), and similar expression products of IC(s). In aspects, EP(s) cause clinically relevant or effective (significantly therapeutic or protective) IR(s) in 2 or 3 such “domains” of the immune system (e.g., promoting T-cell/cellular immunity IR, a B-cell/humoral IR, and an innate adaptive immunity IR).

In aspects, EP(s) (e.g., FP(s)) comprise T cell antigen(s) (Ag(s) comprising T cell epitope(s)). In aspects, EP(s) cause DOS expansion in population, increase in activity, or both, of one or more types of T cells that recognize (are specific for) for Ag(s) in EP(s). In aspects, Ag(s) in EP(s) are recognized by TH1 cells, TC1 cells, or both. In aspects, EP(s) are recognized by immature T cells, differentiated/mature T cells, or both. Typically, EP(s) promote/induce/enhance a DOS response in mature/differentiated T cell(s), lead to a DOS increase in the number of T cell(s) in a host/culture, or both. In aspects, such T cells comprise CD8+ T cells, CD4+ T cells, regulatory T cells, or combinations. In aspects, such T cells comprise memory T cells (e.g., resident memory T (Trm) cells, such as, e.g., CD45RO, C—C chemokine receptor type 7 (CCR7), L-selectin (CD62L), or CD44 T cells), effector memory T cells (e.g., TEM cells and TEMRA cells, or CD45RO+ T cells), tissue resident memory T cells (TRM T cells) (e.g., ue07, aka CD103+ cells), stem memory TSCM cells, virtual memory T cells, etc. In aspects, EP(s) induce DOS IR(s) in natural killer T cells (NKT cells), mucosal associated invariant (MAI) T cells, or Gamma delta T cells. In aspects, T cell responses are limited to only one or some T cell type(s). In aspects, CEPESC(s)/EP(s) lead to a DOS increase in immunological memory to EP Ag(s) (e.g., by, i.a., a DOS increase(s) in antigen-specific memory cells). In aspects, CEPESC(s)/EP(s) having combinations of ≥2, ≥3, 4, 5, or 6 of various element(s) disclosed herein (e.g., a CEPESC comprising CaPNP-associated NAM(s) comprising NS(s) including gDAgFP-ES, polyUB, EEI, and EAT-2 PPT-ES) result in increased immunological memory compared to similar CEPESC(s)/EP(s) lacking one or more of the element(s).

In aspects, T cell antigens of FP(s)/CEPs comprise, PC, GCO, or consist only of ≥1 TH (CD4+)/MHC II antigens, one or more CD8+/CTL/MHC I antigens, or a combination of ≥1 of each such type of T cell antigen. T cell antigens in FPs/CEPs can be associated with DOS promotion/enhancement any suitable type of T cell Ag-associated IR(s). E.g., DOS increases in T cell production of interferon-γ (IFNγ), transforming growth factor-0, IL-4, IL-5, IL-13, IL-17A, IL-17F, IL-21, IL-22, etc. In aspects, EP(s) induce(s) DOS IR(s) in a regulatory T cell, a follicular T cell, or both. In aspects, delivery of an effective amount of CEPESC(s) results in DOS higher number of epitope specific CD8+ T cells in a TR (subject), cell, or population.

In aspects, a fusion protein of the invention will also comprise one or more antigens that lead to effective non-classical MHC I Ag presentation (PMCs are provided in e.g., Liu Y et al. J Clin Invest. 2011; 121(1):249-264 and Lee D J et al. J Exp Med. 1998; 187(3):433-438). In other aspects, CEPESCs lack any non-classical MHC interacting EP ES(s).

In aspects, EP(s) comprise MHC I epitope(s). In aspects, OSMGAOA MHC I epitopes in FP(s) comprise a continuous (linear) AARS of about 8 to about 11 amino acids in length. In aspects, the MHC I antigen detectably or significantly increases CD8+ T cell activity, such as CTL cytotoxicity. In aspects, one, some, most, generally all, or all of the MHC I epitopes will bind one or more MHC I molecules with sufficient affinity to induce MHC I-associated IR(s) (e.g., with affinities similar to those described in Sette A et al. J Immunol. 1994 Dec. 15; 153(12):5586-92 and Berzofsky J A et al. Nat Rev Immunol. 2001 December; 1(3):209-19)).

In aspects, CEPESCs comprise NS(s) encoding ≥1 gDAgFP or ≥1 Ags and ≥1 gDP(s) optimized for expression in a HVEM-expressing host, and (a) the host is not CI immunosuppressed when the method is performed (e.g., the method is performed to provide an immunization effect in the host), (b) no CTL for SMGAOA of the Ags exist in the TR prior to administration, or (c) both, wherein delivery of an EA of the CEPESC results in an effective MHC I response.

FPs/CEPs can comprise confirmed MHC I antigen(s), predicted MHC I antigen(s), or both (optionally in combination with confirmed MHC II antigen(s), predicted MHC II antigen(s), or both). In aspects, predicted MHC I or MHC II Ag(s) comprise a portion of a DCA-associated PPT, e.g., an AARS comprising aromatic amino acid residue(s), Lys or Met residue(s), or a combination. E.g., in aspects 4-6 residues of a predicted antigen comprise aromatic, Met, or Lys residues. In aspects, predicted MHC I Ag(s) include a structure according to the formula Xaa(a)-Xaa4-Xaa5-Xaa6-Xaa(b), where Xaa(a) includes 1-3 of any AAs, Xaa(b) comprises 2⁻⁵ of any AAs, and at least one of Xaa4, Xaa5, and Xaa6 (if not 2 or 3 thereof) are Trp, Phe, Met, or Lys. In aspects, FPs/CEPs include MHC I epitope(s) that are ≥11 AAs in length (non-canonical MHC I epitopes) (SFE Josephs™ et al. Biol Chem. 2017; 398(9):1027-1036).

In aspects, EP(s)/FP(s) comprise(s) at least one long predicted/known T cell antigen (a sequence of over 30, over 40, or over 50 amino acid residues), at least one short known/predicted antigen, or both. In aspects, inclusion of long antigen(s) results in DOS faster T cell response, enhanced dendritic cell processing of the fusion protein, or both. In aspects, FPs/CEPs comprise multiple MHC I antigens (e.g., at least 2, 3, 4, 5, 7, 8, 10, or more MHC I antigens), compositions comprise NS(s) comprising multiple MHC I AgES(s), or both, typically such multiple MHC I Ag(s) are sufficiently different from each other induce different IR(s). In aspects, such Ags exhibit ≤ about 60%, ≤˜45%, ≤˜33%, or ≤˜20% identity to one another. In such an aspect, the inclusion of such multiple MHC I antigens DOS results in a larger proportion of a population exhibiting IR(s) to one, some, most, generally all, or all of such MHC I antigen sequences.

In aspects, FPs/CEPs comprise promiscuous MHC I antigen(s) (which detectably bind ≥2 types of MHC I molecules). In aspects, EP(s)/FP(s) comprise Ag(s) comprising MHC I antigen “supermotifs” that bind a number of common MHC I molecules with high/specific affinity. (SFE Sydney J. et al. Immunol Today. 1996; 17(6):261-6; WO1997033602; and Tang, et al. Immunol Invest. 2003; 32(1-2):31-41; and Doytchinova et al. Methods. 2004; 34(4):444-453). In aspects, FP(s)/EP(s) comprise promiscuous (or “universal” or “broad-range”) epitope(s), which bind a substantial fraction of a particular class of MHC molecules in a species/population (see, e.g., U.S. Pat. Nos. 6,143,935; 6,143,517; 6,689,363, 8,168,201; US20040258660; US20020076416; Waheed Y et al. Asian Pac J Trop Med. 2017; 10(8):760-764.; Subramanian N et al. Asian Pac J Cancer Prev. 2013; 14(7):4167-4175; Shehzadi A et al. Virol J. 2011; 8:55; & Dar H et al. Asian Pac J Trop Med. 2016; 9(9):844-850).

In aspects, FPs/CEPs comprise MHC II T cell epitope(s). In cases, FP(s)/CEP(s) comprise MHC II Ag(s) and MHC I Ag(s). In aspects, either type of such CEP/FP, etc., DOS increase IC proliferation/activity, production of cytokine(s) (as described elsewhere—e.g., of IFNg), IR(s), etc. In aspects, CEP(s)/FP(s) comprise predicted/putative MHCII epitope(s) (e.g., a DCA-associated surface exposed Ag or predicted surface Ag, in aspects comprising a structure associated with MHCII epitopes such as a coiled secondary structure, aromatic residue(s), Lys/Met residue(s), lack of glycosylation site(s), etc.), known MHCII epitope(s), or both. In aspects, EP(s)/FP(s) comprise MHC II antigen(s) or putative MHC II Ag(s) that also comprise an AARS or AA(s) associated with neutralizing Ab epitope(s).

In aspects, CEPs/FPs comprise multiple MHC II Ag(s), multiple putative MHC II Ag(s), or both. In aspects, FPs/CEPs also (i.e., also/alternatively) comprise multiple MHC I Ag(s), multiple putative MHC I Ag(s), or both. In aspects, OSMGASAOA of MHCI/MHCII Ag(s) are associated with (a) a linker sequence, e.g., a medium linker (of at least four residues), a flexible linker, a linker comprising a glycosylation site, a cleavable linker, a linker comprising a cleavage site (e.g., a 2A site), etc., (b) an intracellular targeting sequence (e.g., a PTPS, an ERTPS, or an exosome-targeting sequence, etc.), or (c) both. In aspects, inclusion of ≥2, ≥3, ≥4, ≥5 Ag(s), etc., in EP(s) DOS increase(s) the range of IR(s), quality of IR(s), or both.

In aspects, a CEP/fusion protein comprises promiscuous/universal MHC II epitope(s), MHC II supermotif(s), or both.

In aspects, a CEP/FP comprises MHC I promiscuous epitope(s)/supermotif(s), MHC II supermotif(s)/promiscuous epitopes or supermotifs, or both (in aspects some, most, or all thereof are FFs or FVs). PMCs are provided in Kashyap M et al. Infect Genet Evol. 2017; 53:107-115; Grabowska A K et al. Int J Cancer. 2015; 136(1):212-224; Rosa D S et al. Arch Immunol Ther Exp (Warsz). 2010; 58(2):121-130; Malcherek G et al. J Exp Med. 1995; 181(2):527-536; Sinigaglia F, et al. J Exp Med. 1995; 181(2):449-451; and Morse H C. Immunol Today. 1996; 17(1):47-48. In aspects, OSMGASAOA Ag AARS(s) in EP(s), FP(s), or CEP(s) are ˜15-60 , ˜15-45, about 15-30 , ˜20-50, about 20-45, about 20-40, or about 20-30 AARSs in length. In aspects, EP(s)/FP(s) comprising MHCI or MHCII epitopes also induce humoral IR(s). In aspects, CEPs/FPs lack any B-cell epitopes (BCEs) but still result(s) in a DOS enhancement of humoral response(s).

In aspects, EP(s)/FP(s) comprise Ags including overlapping epitope(s), e.g., overlapping MHC I and MHC II epitopes. PMCs are provided in Lohia N et al. Viral Immunol. 2014; 27(5):225-234; Bristol J A et al. Cell Immunol. 2000; 205(2):73-83; and Fayolle C, et al. J Immunol. 1991; 147(12):4069-4073).

In aspects, FPs/CEPS comprise MHC II antigen(s) that are cross-presented as MHC I antigens. PMCs are provided in, e.g., Tan A C et al. Immunol Cell Biol. 2013 January; 91(1):96-104 and Dickgreber N, et al. J Immunol. 2009; 182(3):1260-1269. In aspects, Ag(s) are variants (FV(s)) modified to promote cross-presentation (e.g., by incorporation of a phosphatidylserine-binding domain, described in US20190218260).

In aspects, one, some, most, generally all, or all (OSMGAOA) CD4 TCEs in EP(s)/FP(s)/CEPs are Th1 (T helper cell type 1) TCEs. In aspects, OSMGAOA CD4 TCEs in EP(s)/FP(s) are Th2 (T helper cell type 2) TCEs. In aspects, OSMGAOA CD4 TCEs in CEPs are Th17 TCEs. In aspects, CEPs comprise ≥1, ≥2, ≥3, ≥4, or ≥5 Th1 TCEs, Th2 TCEs, Th17 TCEs, or a mix thereof. In aspects, OSMGAOA of such TCEs induce IRs against the same type of DCA or same DCA. In aspects, ≥1 TCE(s) are contained in gDAgFP(s). In aspects, ≥2 Th1 TCEs, ≥2 Th2 TCEs, or ≥2 of both is/are contained in gDAgFP(s). In aspects, CEPs comprise gDAgFP(s) comprising polypepitope(s) (PE(s)) comprising ≥1 Th1, ≥1 Th2, or ≥1 Th17 TCEs. In aspects, gDAgFP(s) in CEPs comprise ≥2 of Th1, Th2, or Th17 TCEs (e.g., at least 2 of such types or at least 1 Ag of each of these types). In aspects, PEs, such as these PEs, comprise MSL(s), FL(s), or MSFL(s), or self-cleavage site(s). In aspects, CEPs include Th17 TCE(s) & SMGAOA of the TCEs are anti-cancer TCEs or anti-fungal TCEs. In aspects, OSMGAOA CD4 TCEs/TCEs are Th2 TCEs and SMGAOA of such TCEs induce IRs against extracellular parasites, such as helminth(s).

In aspects, CEPs comprise ≥1 or ≥2 Th2 TCEs and IRs comprise DOS enhanced production of IL-4, IL-5, IL-6, IL-10, or IL-13, or combinations. In aspects, CEPs comprise ≥1 or ≥2 Th1 TCEs and IRs comprise DOS enhanced IFNg expression, IL-2 expression, or both. In aspects, the delivery of a particular TH epitope type DOS enhances the level of memory T cells of that TH type in TRs (e.g., Th1 TCE CEPs DOS enhance related Th1 memory cells).

In cases step(s) or element(s) are applied/added to enhance Th1, Th2, or Th17 IRs (e.g., optimizing Ag doses/Ag affinity, such as causing high/low Ag expression to promote Th2 responses, use of low affinity TCEs to promote Th2 IRs, etc. PMCs related to promoting polarization towards TH response(s) are known (SFE Kaiko G E et al. Immunology. 2008; 123(3):326-338; Rogers P R et al. J ImunnnoL 2000; 164(6):2955-2963; Brandt K et al. Scand J Immunol. 2002:56(6):572-579; and Rogers P R et al. J Immunol. 1999:163(3):1205-1213). In cases cytokine(s), trafficking signal(s), etc., also are expressed/provided, to promote particular types of Th responses (cytokines are discussed elsewhere, MHC trafficking signals are exemplified in WO2019071032). In cases, EP(s) including Th1 TCEs, Th2 TCEs, or both include or are AAW toll like receptor (TLR) modulators

In methods, two or more different CEPESCs are administered to TRs, in one of which the CD4 TCEs or TCEs primarily comprise, generally consist of, substantially consist of, or consist of (PCGCOSCO or CO) Th1 TCEs against a DCA or group of related DCAs, and at least one other of which the CD4 TCEs or TCEs PCGCOSCO or CO Th2 TCEs against a DCA or group of related DCAs. In aspects, delivery of the second CEPESC(s) in such a method is an optional step, depending on CEs in the TR(s) receiving the first CEPESC(s). In aspects, such methods are used to treat cancer.

In aspects, FPs/CEPs also comprise BCE(s). In aspects, FP(s)/CEPs comprise ≥2 BCEs. In aspects, BCEs comprise linear B cell epitope(s). In aspects, BCEs of CEPs PCGCOSCO or CO linear BCEs. In aspects, CEP(s) also comprise ≥1 or ≥2 non-linear/conformational (discontinuous) B cell epitopes.

In aspects, CEPs comprise no known B cell epitopes. In aspects, EPs or CEPs lack BCEs but nonetheless DOS induce BC IR(s) in TR(s).

In aspects, CEPESCs comprise 1+ putative B cell epitope-containing sequences. In aspects, such a putative B cell antigenic sequence is from a surface exposed sequence, primarily comprises hydrophobic amino acids, or both. In another aspect such a putative B cell epitope also reflects a predicted B cell epitope identified by BCE prediction methods.

In aspects, Ag AARS(s) comprise flanking sequence(s) upstream or downstream of known/predicted epitope(s) or a core Ag sequence (having characteristics described elsewhere, e.g., about 20-45, 20-35, 7-20, 8-20, 8-15, or !8-12 AARs). Each flanking sequence can comprise, e.g., ˜10⁻³⁰ amino acid residues, e.g., 5-25 flanking residues. In aspects, the flanking sequences are identical or at least related (e.g., VR or SI) to native sequence(s) flanking the antigen in its WT context. Thus, for example, a fusion protein of the invention can comprise ≥2, 3, 4, 5, 6, 7, 8, 9, 10, 11, 12, 13, 14, 15, 16, 17, 18, 19, or 20 flanking residues, such as 2-30 flanking residues, 4-28 flanking residues, 4-24 flanking residues, or 5-20 flanking residues, which optionally are identical or substantially identical to corresponding flanking residues of the epitope or epitope-related sequence in a naturally occurring protein. In aspects, the flanking residues are both upstream and downstream of the epitope. In aspects, the presence of the flanking residues associated with an MHC II epitope DOS MHC affinity, TCR recognition, IR(s), etc. In aspects, inclusion of flanking sequences of an MHC I epitope DOS enhance proteasomal processing, ER processing, T cell recognition, IR(s), etc. PMCs are provided in, e.g., Luc Teyton. J Clin Invest. 2007; 117(11):3164-3166.

As noted, in aspects EPs comprise ≥2 Ag sequences, e.g., ≥3, ≥4, ≥5, ≥7, ≥8, or ≥10 separate and distinct Ag sequences (e.g., one or more gD: Ag fusion proteins can comprise 2-20, 2-16, 2-12, 2-10, 2-8, or 2⁻⁵ antigenic AARS(s), each comprising epitope(s), e.g., ≥2 epitopes). EPs comprising ≥2 epitopes are referred to as “polyepitope” FPs or polypepitope(s) (PE(s)). In aspects, one, some, most, generally all, or all (OSMGAOA) of ≥2 antigenic sequences of FP(s) are from different proteins of a DCA, different types of a DCA, or both. In aspects, a polyepitope comprises 2 Ags that are non-heterologous in terms of species, PPT, or both. In aspects, a polyepitope FP comprises 2 Ags against 2 DCAs. In aspects, such Ags may be associated with different DCAs associated with a single disease (e.g., a cancer and a cancer-promoting virus, such as HPV). In aspects, the polyepitope vaccine comprises one or more linkers (e.g., one or more mid-sized, flexible, or mid-sized and flexible linkers, e.g., GPGPG (SEQ ID NO:730), ITS(s) (e.g., one or more PTPSs), or both, associated with OSMGAOA Ags in polyepitope(s). In aspects, the polyepitope sequence comprises both MHC I and MHC II antigens. In aspects, polyepitope(s) DOS causes/enhances CTL, Th, or B cell IR(s). In aspects, PE(s) include AgV(s) (“antigenic variants”), e.g., GSRV(s). PE PMC(s) are described in, e.g., Zhang Cell Mol Immunol. 2018; 15(2):182-184; Thomson et al. J Immunol. 1996; 157(2):822-826; Smith Curr Opin Mol Ther. 1999; 1(1):10⁻¹⁵; Suhrbier Immunol Cell Biol. 1997; 75(4):402-408; Livingston et al. J Immunol. 2002; 168(11):5499-5506; Antonets D V et al. BMC Res Notes. 2013; 6:407, and Patronov A et al. Open Biol. 2013; 3(1):120139. Methods of epitope identification are described in US20200325182.

In aspects, CEPs/EPs, comprise immunodominant epitope(s), subdominant epitope(s), or both (see US 20200325182 and Le Gall S et al. J Clin Invest. 2007; 117(11):3563-3575). See also US20200325182. In aspects, FP(s) comprise flanking sequences, positioning of the Ag in the FP (e.g., positioning at or near the N or C terminus of an FP, such as within 1-15% of the terminal residues), inclusion of cleavage site(s) near Ag(s), variations in Ag sequences, or both, to DOS increase or decrease immunodominance of epitope(s). In aspects, FP(s)/EP(s) comprise subdominant epitope(s). IN aspects, subdominant epitope(s) result in DOS reduced Ag immune tolerance, prolonged IR(s), increases the proportion of subjects that exhibit a clinically relevant response to CEP, or a combination. In aspects, EP(s) comprise a mix of both dominant and subdominant epitopes, in aspects wherein immunodominance is not significant enough to silence all, generally all, most, some, or at least one of the subdominant epitope(s) in the EP(s). PMC(s) are provided in Dominguez M R, et al. PLoS One. 2011; 6(7): e22011; Filskov J et al. J Virol. 2017; 91(14): e00130-17; Ascough, S et al. Frontiers in Microbiology, 6(JAN); and Ling Chen et al. PNAS March 2018, 115 (12) 3126-3131). Examples of subdominant and dominant epitopes are provided in US20200325182.

In aspects, EP(s) comprise cryptic epitope(s) (aka, neo-epitope(s)/masked epitope(s)), unnatural immunity epitopes, or both. PMCs related to cryptic epitopes are provided in, e.g., David et al., JBC, 2001, 6370-6377; Matsuura et al., International Immunology, 2000, 1183-1192; Rasheed et al., Life Sciences 79 (2000), 2320-2328). Unnatural immunity is described in Nabel G J, Fauci A S. Nat Med. 2010; 16(12):1389-1391.

In aspects, EP(s) comprise variant(s) of naturally occurring antigen(s)/antigenic sequence(s) (“antigenic variant(s)” or “AgV(s)”). In aspects, AgV(s) comprise editope(s). An “editope” is an AgV that exhibits enhanced IR(s) in context(s) when expressed in TR(s)/cell(s).

In aspects, EP(s) comprise TCE(s) comprising variations (e.g., substitution(s) that DOS modify MHC binding affinity, e.g., in aspects in an otherwise subdominant, cryptic, or poorly immunogenic epitope. In aspects, such an editope comprises one or more substitutions in anchor residues (PMCs provided in Hobohm U et al. Eur J Immunol. 1993; 23(6):1271-1276; Chujoh Y et al. Tissue Antigens. 1998; 52(6):501-509; Huang J et al. BMC Immunol. 2012; 13:50; Dobano C, et al. Mol Immunol. 2007; 44(9):2235-2248; Vierboom M P et al. J Immunother. 1998; 21(6):399-408; and Gerner W et al. J Virol. 2009; 83(9):4039-4050). Enhanced MHC affinity epitopes adaptable to aspects are described in, e.g., Tine J A et al. Vaccine. 2005; 23(8):1085-1091; Hofmann S, et al. Cancer Immunol Immunother. 2015; 64(11):1357-1367; and U.S. Pat. No. 7,605,227. In aspects, epitope(s) enhancement is with respect to binding a particular type of MHC, such as class II DR4. In aspects, a dominant epitope is substituted with a less immunogenic editope in EP(s), particularly in a multi-Ag method or multi-Ag expression product, such as a multi-Ag gdAgFP, resulting in detectably broader/improved IR(s). Reduced immunogenicity editopes have been demonstrated by, e.g., Ruckwardt T J et al. J Immunol. 2010; 185(8):4673-4680. Other principles relative to modification of editopes impacting the dominance hierarchy are described in, e.g., Sadegh-Nasseri S et al. Mol Immunol. 2019; 113:115-119. Methods of screening/generating editopes is described in US20200325182.

In aspects, editope(s) in EP(s) are formed by modifying immunodominant non-protective epitope(s) (IDNPE(s)). Changes to IDNPE(s) can induce a new hierarchy of immune responses at either or both the B and T cell levels (SFE Garrity et al., J. Immunol. (1997) 159(1):279-89) against subdominant or previously silent epitopes. Such methods are often referred to as “immune refocusing” methods.

In aspects, Ag(s) or other EP(s) comprise variations that DOS reduce the immunogenicity. Numerous strategies for deimmunization of can be used. In aspects, OSMOA AARSs in EPs comprise deimmunized variant(s)/variation(s) (DIV(s)). In aspects, OSMOA undesirable epitopes in WTC AARS(s) of EP(s) are subject to substitutions, deletions, combinations, etc., resulting in functional DIVs. In aspects, decoy antigen(s), overlapping epitope(s), immunodominance, Th2/Th17 epitope(s), undesirable BCE(s), etc., are removed. In aspects, DIV(s) in EP(s) DOS reduce anti-biotherapeutic immune response(s) (aBIR(s)) associated with WTC AARS(s) or that arise from fusion of non-homologous AARS(s). In aspects, DIVs are generated through “humanization” (relevant PMCs are provided in, e.g., Jones et al., Nature 321:522-525 (1986); Riechmann et al., Nature 332:323-327 (1988); Verhoeyen, et al., Science, 239:1534-1536 (1988); and Chen J, et al. Int J Biol Macromol. 2016 January; 82:522-9).

In aspects, DIV(s) comprise substitutions or deletions that eliminate undesired TCE(s). In aspects, TCE DIS(s) exhibit DOS reduced binding to MHC(s). Methods for deimmunizing AARS(s) are provided in US20200325182. One such method is the use of the IEDB deimmunization tool. Applying such analysis to, e.g., BHV-1 gD, identifies an immunogenic sequence at AAs 155-169 of iBHV-1 gD (SEQ ID NO:646) and possible variations thereof, e.g., FVs at AA M⁻¹⁷³, e.g., DDELGLPMAAPARLV (SEQ ID NO:647), DDELGLGMAAPARLV (SEQ ID NO:648), and DDELGLDMAAPARLV (SEQ ID NO:649) that are predicted to be less immunogenic (points of variation underlined). Applying the same method to murine EAT-2 (mEAT-2), identifies the sequence at AAs 91-105 (QGLVVHLSNPIMRNN (SEQ ID NO:650)) as immunogenic and recommends variations at the 2^{nd} Val, 2^{nd} Leu, or Met of the AARS (e.g., QGLVDHLSNPIMRNN (SEQ ID NO:651), QGLVVHLSNPDMRNN (SEQ ID NO:652), or QGLVVHDSNPIMRNN (SEQ ID NO:653) to reduce IR(s).

Another type of DIV is a de-immunodominance DIV (DEDIV). DEDIV(s) comprise modifications that reduce the immunodominant effects of a BCE or TCE. In aspects, DEDIVs DOS reduce immunodominance blocking other subdominant epitope(s) (e.g., cryptic TCE(s)). In aspects, immunodominance is enhanced or reduced in EP(s) through the use or non-use of ITS(s). E.g., an Ag can be directed to an exosome over an immunoproteasome to reduce immunodominance, or an Ag can be directed to an immunoproteasome to enhance immunodominance. Such methods are exemplified in, e.g., Dzutsev A H et al. Int Immunol. 2007; 19(4):497-507. In aspects, enhanced MHC affinity of TCEs results in lower immunogenicity of PPTs (Id). In aspects, immunodominance or immunogenicity is blocked in a DIV, i.a., by introduction of modifications (e.g., cys-cys bridges) that block access to an epitope (exemplified in Rouvinski A et al. Nat Commun. 2017; 8:15411. Published 2017 May 23). In aspects, epitope immunodominance or immunogenicity is modified by elimination of a domain (e.g., the HA head of influenza), unmasking subdominant epitopes (e.g., through removal of glycosylation sites, cys-cys bridges, and the like), or enhancing immunogenicity of a subdominant epitope (e.g., through hyperglycosylating a BCE, enhancing MHC binding in a TCE, or combining part of an epitope with other AARS(s), etc. Such approaches are reviewed in, e.g., Mathew N R, Front Immunol. 2020; 10:2997. In aspects, an immunodominant epitope is substituted with a mimetic with reduced immunodominance. See, de Taeye S W et al. J Biol Chem. 2018; 293(5):1688-1701. In aspects, increasing epitope diversity in EP(s) reduce(s) immunodominance (SFE Woodruff M C et al. Cell Rep. 2018; 25(2):321-327.e3).

In aspects, DIV(s) at a PPT level comprise adding regulatory T cell (Treg) epitope(s) (TREGE(s)) to EPs, which reduce EP IR(s). In aspects, no TREGEs are present. In aspects, TREGE(s) in EP(s) are removed or the effect of TREGEs (e.g., MHC binding) is reduced. In method aspects, TR(s) can be subjected to methods that induce partial tolerance to PPTs to reduce immunogenicity or to reduce the immunodominant effects of potentially immunodominant epitopes. Such approaches are exemplified by, e.g., Silva M, et al. Cell Rep. 2017; 21(13):3672-3680. In aspects, CEPs comprise IL-10 PPTs (isolated or FPs). In aspects, IL-10 PPTs in CEPs DOS reduce immunodominance effects of OSMOA potentially immunodominant epitope(s) in CEPs (such IL-10 effects are covered in St Leger A J, J Immunol. 2013; 191(5):2258-2265). In aspects, flanking sequences of epitopes are modified to modulate immunodominance. Such an approach is discussed in, e.g., Mo A X, et al. J Immunol. 2000; 164(8):4003-4010.

Undesirable BCEs can be removed by similar methods. Conformational BCEs can be removed by substitution of residues or non-inclusion of some parts of the conformational BCE. Examples of this approach are Schmohl J U et al. Toxins (Basel). 2015 Oct. 10; 7(10):4067-82 and Cantor J R et al. Methods Enzymol. 2012; 502( ):291-319. In other aspects, the number of BCEs are reduced by GSRVs.

In aspects, EP(s) include AgV(s) that comprise variation(s) resulting in removal of glycosylation site(s) (glycosylation site removal variant(s) or GSRV(s)). Ag(s) with GSRV(s) can be called GSRAgV(s). In aspects, OSMGAOA known/potential N-linked glycosylation-associated sequences of Ag(s) are modified (e.g., substituted) to remove the potential/known N-linked glycosylation sites. For example, any or all of the N—X—S sequences, N—X-T sequences, or both, in Ag(s), can be modified by a substitution of the N residue. In aspects, SMGAOA N residues of any substituted sites are substituted with a D residue or an E residue. In aspects, a linker alternatively or also is placed in one or more of such sequences as a substitute for the N residue. Such a linker can be a short linker, such as di-peptide (e.g., Ala-Ala) or tripeptide linker or a mid-sized or longer linker (e.g., a GGGS, GSGS, or GGGG linker). In aspects, at least part(s) of such sites are deleted. In aspects, such modifications result in DOS reduced humoral response to the GSRV EP(s). In aspects, such a reduced humoral response leads to a detectably or significantly longer/sustained IR(s) or clinical improvement(s).

In aspects, AgV(s) include deletions of residue(s) from a WT epitope or Ag sequence, e.g., resulting in removal of decoy antigen(s) (immunodominant epitopes with no known or desired function). In aspects, EP(s) comprise anti-decoy epitope Ab AARS(s). In aspects, EP(s) comprise variations that eliminate a decoy epitope.

Porcine circovirus (PCV) is an example of a virus that expresses a decoy antibody epitope on the surface of the viral particle. Vaccines associated with decoy antigens also are “leaky.” SFE Jin J et al. Biochem Biophys Res Commun. 2018; 496(3):846-851; Yu C et al. Vaccine. 2016; 34(50):6358-6366; and Trible B R et al. Virus Res. 2012; 164(1-2):68-77. In aspects, the decoy PCV Ag is removed, e.g., replaced in the sequence with a different PCV Ag, which may be associated with one or more linkers and connected to other upstream, downstream, or surrounding PCV Ag residues. Another virus comprising at least one decoy epitope is Porcine reproductive and respiratory syndrome virus (PRRSV) (SFE U.S. Pat. No. 9,441,015; Thaa et al. PLoS One. 2013; 8(6): e65548. Published 2013 Jun. 6; and Mateu E et al. Vet J. 2008; 177(3):345-351). In aspects, an EP comprises a PRRSV GP5 Ag sequence that comprises residues normally associated with the GP5 decoy epitope, but wherein the decoy epitope has been removed/replaced.

In aspects, EP(s) (e.g., FP(s)) comprise known antigens, clinically relevant antigens (CRAs), or both. A “clinically relevant antigen” (or “CRA”) is an antigen identified in subjects of a particular species when experiencing a DCA (e.g., in biological samples of DCA-associated material, such as PBMC). Methods of generating CRA(s) and identifying CRA(s) are in US20200325182.

In aspects, EP(s) comprise peptidic checkpoint modulators (PCMs). CCCs and associatively applied compositions (AACs) also can comprise PCMs, PCM-encoding NSs, & other checkpoint modulators (other CMs). PCMs can modulate any such checkpoint pathway cell receptor (CPCR) or any such class of CPCRs. CCCs and AACs can be/comprise any PCM or corresponding PCMESNS that is described in this disclosure (and PCMs can comprise any PCM that corresponds to an immunomodulator (IM) CCC/AAC described herein).

A checkpoint modulator (CM) that DOS modulates an immune checkpoint. CM(s) can modulate checkpoint(s) that are stimulatory, inhibitory, situationally either stimulatory or inhibitory, co-stimulatory, or a suitable combination thereof. In aspects, PCMs/CMs are “immunoactivators” (or immunostimulators) (e.g., agents that, i.a., induce proliferation of IC(s) (e.g., TCs), enhanced IC distribution/motility, enhanced cytokine secretion, etc.). In aspects, CMs (e.g., PCMs), i.a., suppress inhibitory checkpoint pathway(s). Exemplary CMs include PD1 and CTLA-4, lymphocyte activation gene-3 (LAG-3), B and T lymphocyte attenuator (BTLA), programmed death-1 homolog (PD-1H), T-cell immunoglobulin and immunoreceptor tyrosine-based inhibitory motif domain (TIM-3)/carcinoembryonic antigen cell adhesion molecule 1 (CEACAMI), VISTA, and the poliovirus receptor (PVR)-like receptors. SFE Torphy R J et al. Int J Mol Sci. 2017; 18(12):2642, and Zahavi D J et al. Int J Mol Sci. 2019; 20(1):158. CEPs can comprise ≥2, ≥3, ≥4, or ≥5 PCMs of any suitable type of CM. In aspects, ≥2 PCMs are expressed as separate PPTs. In aspects, a FP comprises ≥2 PCM AARSs. In aspects, OSMGAOA PCM EPs are non-fusion protein PPTs (“NFPs”). In aspects, CEPs comprise a mix of PCM FPs and NFPs.

In aspects, OSMGAOA PCM(s) in CEPs are checkpoint pathway (CP) receptor ligands (RLs) (CPRLs), immune cell receptors (ICRs), or both. In aspects, OSMGAOA PCM(s) are CPCRs or CPRLs; ICRs or ICR ligands (ICRLs); or a combination. In cases, PCMs comprise forms that can be classified as CPCRs/CPRLs (e.g., VCAM⁻¹ can either be a cell membrane bound PPT or a soluble ligand for its related receptor). Typically, PCM(s) can also be characterized as being TS(s). E.g., OSMGAOA PCM(s) that are ICRL(s) are also ICTS(s) with ICR RBDs. In aspects, OSMGAOA CEP PCM(s) or CCC/AAC IMs further can be classified as agonists, antagonists, partial agonists, partial antagonists or combinations thereof. In aspects, OSMGAOA PCM(s) in a CEP comprise Abs or Ab FP. In aspects PCMs, such as PCM Ab PPTs can be multimeric, multivalent, multi-specific, or a combination of some or all thereof. Examples of immunostimulatory Ab(s) include anti-CD40L Abs, anti-OX40 Abs, anti-CD2 Abs, anti-CD28 Abs, anti-CD137 Abs, anti-CTLA4 Abs, anti-PD-1 Abs, anti-PD-L1/PD-L2 Ab, or anti-ICOS Abs. In aspects, OSMGAOA PCMs in a CEP are non-Ab PPTs. In aspects, OSMGAOA PCMs in CEPs are non-Ab EPs that are multimeric, multivalent, multi-specific, or exhibit a combination thereof. In aspects, PCM(s) are monospecific, but multivalent (e.g., PCMs can be trap protein(s), such as a trap protein specific for a checkpoint ICR).

In aspects, a PCM is a co-stimulatory molecule (CSM) ICR or CSM ICRL (e.g., B7, ICAM⁻¹, LFA-3/CD58, 4-1BBL, CD59, CD40, CD70, VCAM⁻¹, or OX-40L, or a ligand thereof). In aspects, a PIM also can comprise a PPT or AARS of a T cell costimulatory molecule, such as CD28, CD40, or ICOS or a ligand or other immunomodulator thereof. In aspects, a PIM binds a co-stimulatory CD80 receptor, a CD86 receptor, or a CD46 receptor.

In aspects, a PCM is an inhibitor of IDO1, ID02, TD02, or A2aR. In aspects, the PIM is or is a ligand or adaptor for PD-L2 (B7-DC, CD273), TIM4, 2B4, B7-H2, B7-H3, B7-H4, B7-H6, CD2, CD27, CD28, CD30, CD30L, CD40, CD40L, CD48, CD58, CD70, CD80, CD86, CD96, CD112, CD113, CD137, CD137L, CD155, CD160, CD226, CD276, CRTAM, DR3, GAL9, GITR, GITRL, HAVCR2, HVEM, IDOl, ID02, ICOSL, ILT3, ILT4, LAIR1, LIGHT, LTBR, MARCO (macrophage receptor with collagenous structure), PS (phosphatidylserine), OX40L, SLAM, TD02, TL1A, VISTA, VTCN1, or any combinations thereof.

In aspects, OSMOA PCMs are immunoreceptor tyrosine-based inhibition motif (ITIM) receptor modulator(s). In aspects, a PCM blocks binding/activity of an ITIM receptor. Examples of ITIM receptors include NKG2A/CD94, PD-1, LIRs, SIRPu, TIGIT, and KIRs. In aspects, OSMOA PICCMs are non-ITIM CM receptor modulators. Examples of non-ITIM CM receptors include CTLA-4, LAG-3, TIM-3, and CD200R. In aspects, CEPs comprise both ITIM ICR and non-ITM ICR modulator(s). ITIM receptors/modulators can be stimulatory/inhibitory.

In aspects, OSMOA PCMs are immunoreceptor tyrosine-based activation motif (ITAM) receptor modulators. In aspects, a PCM binds and activates an ITAM receptor. Examples of ITAM receptors include CD16, NKp30, NKp46, NKG2D, and DNAM⁻¹. Typically, ITAM receptors are stimulatory. In some cases, ITAM receptors are inhibitory.

In aspects, PCMs exhibit DOS effects in both ICs & non-IC cells.

In aspects, OSMGAOA PCMs in CEPs exhibit significant IRs primarily, generally only, substantially only, or only in NKCs. Examples of PCMs that exhibit significant IRs in NKCs include modulators of activating NKC CPCRs e.g., CD94-NKG2C/E/H heterodimeric receptors; NKG2D; activating KIRs; natural cytotoxicity receptors such as NKp30, NKp44, and NKp46; & the nectin/nectin-like binding receptors DNAM⁻¹/CD226 and CRTAM and NKC receptors (NKCRs) that inhibit NKC activation including inhibitory KIRs; CD94-NKG2A; & the nectin/nectin-like binding receptors TIGIT and CD96. Other NKCRs that regulate NKC activation include SLAM family receptors including 2B4/CD244, CRACC/SLAMF7, and NTB-A/SLAMF6, as well as Fc gamma RIIIA/CD16a, CD27, CD100/Semaphorin 4D, and CD160. The sialic acid-binding Siglecs (Siglec-3,-7, & -9), ILT2/LILRB1, KLRG1, LAIR-1, CD161/NKR-PiA, and CEACAM⁻¹ are also NK cell inhibitory receptors. PVR is another NKC-associated CPCR (Xu et al. Cancer Immunol Immunother. 2017; 66(10):1367-1375. Inhibitory T-cell receptors also present in NKCs include CTLA-4, PD-1, T cell immunoglobulin- and mucin-domain-containing molecule 3 (TIM-3), lymphocyte activation gene 3 (LAG-3), & TC immunoreceptor with Ig and ITIM domains (TIGIT). Relevant principles, etc. (PMCs) are in Beldi-Ferchiou et al. Int J Mol Sci. 2017; 18(10):2129 and Sun H, Front Immunol. 2019; 10:2354.

In aspects, OSMGAOA PCM(s) DOS modulate CTLs or NKCs (or generally or only modulate CTLs or NKCs). Examples of such ICRs include PD-1, LAG-3, TIGIT, and TIM-3 receptors. PCMs that modulate CPCR(s) common to NKCs and T-cells include TIGIT, NKG2A, & inhibitor KIRs or NKCs. In aspects, PCMs DOS modulate activity in T-cells and NKT cells. Examples of such PCMs include modulators of PD-1, LAG-3, and TIM-3. In aspects, PCMs(s) are in ≥2, ≥3, ≥4, or all of DCs, NKCs, INKT cell, γδ T cells, or B-1 B cells.

In aspects a PCM binds to a receptor expressed in epithelial cells, fibroblast cells, or both types of cells and induces IRs therein. Although not classified as ICs, such cells can be involved in IRs. E.g., epithelial cells can contribute to IRs and given their abundance can play a significant role in CEs. An example of such PCMs include HVEM-binding gDPs. Another checkpoint pathway involving many non-IC cells is the fibroblast activation protein-alpha (FAP) checkpoint pathway. SFE Chen L et al. Biochem Biophys Res Commun. 2017; 487(1):8-14. In aspects, PCMs comprise FAP PPTs or modulators of a FAP pathway. The use of FAP checkpoint modulators with gDAgFPs is exemplified in some of the Wistar Art and such approaches can be adapted to use in any of the improved/novel CEPESCs described herein. In aspects, PCMs do not comprise FAP PPTs or FAP modulators. In aspects, PCMs primarily, generally only, substantially only, or only exhibit significant IR(s) in ICs.

Examples of modulators that exhibit primarily ITIC IRs, particularly primarily NKC and DC IRs, include SLAM receptor modulators (e.g., SLAM CPRLs such as CRACC modulators, e.g., CRACC inhibitors, and SLAM STAPs, such as EAT-2 PPTs), modulators of KIRs, modulators of NKG2Ds, and modulators of STING PPTs.

In aspects, OSMGAOA of the PCMs in CEPs exhibit IRs, are expressed endogenously, or both, primarily, generally only, substantially only, or only in (1) DCs, (2) in DCs and T-cells, (3) in DCs and NKCs, or (4) in DCs, NKCs, and T-cells. Examples of such PCMs include modulators of PD-1/PD-L1 PPTs, ILT2 modulators, and TIM-3 modulators. In aspects, OSMGAOA PCMs are PPTs corresponding to DC PPTs expressed only (or significantly more expressed) upon DC stimulation (e.g., DC-TLR stimulation), such as PD-L1 PPTs or ILT2 PPTs, or modulators thereof.

In aspects, a PCM is a PPT that has an expression induced by APC (e.g., DC) activation/maturation or is an FF or FV thereof. In aspects, a PCM is a CPCR or CPRL primarily expressed on APCs, such as DCs, e.g., GITRL. In aspects, a PCM is a soluble isoform of a WT PPT having other isoforms that are expressed as CPCRs. An example of such a PCM is a soluble canine CD80 isoform, a soluble human CD80 isoform, or a canine CD86 soluble isoform (or FFs or FVs thereof).

In aspects, CEPs include ≥2 PCMs that are heterologous. In aspects, PCMs include ≥2 PCMs that modulate pathways of different CPCR families (discussed below). Examples of such aspects include combination of PD-1 pathway blocking PCMs with CD20-blocking PCMs, inhibitory KIR-blocking PCMs, TIGIT blocking PCMs, and the like. Additional examples of such combinations are mentioned below.

In aspects, PCMs DOS result in reduced T-cell anergy, reduced IC tolerance, enhanced IRs (including proliferation of ICs, activity of ICs, or both), enhanced CEs, and combinations of any thereof.

In aspects, a PCM exhibits DOS IRs in DCs. In aspects, OSMGAOA PCMs of a CEP primarily, generally, substantially only, or only exhibit significant IRs in DCs. In aspects, OSMGAOA PCMs primarily, generally, substantially only, or only exhibit significant IRs in DCs and other ITICs (in aspects only DCs and NKCs). In aspects, PCM(s) of CEPs exhibit DOS IRs in ≥2 of DCs, T-cells, and NKCs (e.g., all 3 cell types). In aspects, PCM(s) of CEPs exhibit DOS greater IRs in DCs than in other cells. In aspects, PCM(s) exhibit DOS more IRs in innate trained immunity cells (ITICs) than other cells or only exhibit DOS IRs in ITICs (such PCMs can be referred to as “ITIIMsi” (innate trained immunity immunomodulators or ITIPIMs) herein). In aspects, PCM(s) exhibit DOS IRs in ICs, comprising DCs than other cells. Examples of PCMs that exhibit significant IRs in DCs include modulators of DEC-205, Langerin, Clec9A, DC-SIGN216, and MR217, and other DC ICRs described herein. In aspects, modulators of any such ICR(s) can be PCMs. PMCs relevant to PCMs and other CIs are described in, e.g., De Sousa Linhares A et al. 2018; 9:1909; Khan M et al. Front Immunol. 2020; 11:167; and Sun H et al. Front Immunol. 2019; 10:2354.

In aspects, OSMGAOA EPs in a CEP are internal target modulators or more specifically internal target immunomodulators (ITMs/ITIMs). Most ITMs are ITIMs. Each term provides implicit support for aspects incorporating the other term herein. In aspects, ITM(s) modulate a checkpoint pathway, e.g., by directly interacting with a CPCR or another factor downstream of a checkpoint pathway signaling cascade or upstream of a checkpoint cascade (e.g., a transcription factor). However, in aspects ITM(s) can include PPT(s) that are not associated directly or otherwise with checkpoint modulation.

In aspects, ITIMs/ITIPIMs in CEPs include modulators of phosphoinositide-3 kinase (PI3K)/Akt/mTor (“PAM”) pathway/cascade system. In aspects, ITIMs include PAM pathway inhibitors. Examples of such PAM pathway inhibitors include YVPGP (SEQ ID NO:731), P6-55 (Guo W et al. Cancer Lett. 2017; 405:1-9), and the peptides described in WO2016103176. In aspects, such an inhibitor is a PTEN PPT (SFE Dillon L M, Miller T W. Therapeutic targeting of cancers with loss of PTEN function. Curr Drug Targets. 2014; 15(1):65-79). Additional examples of ITIMs are agents that block IκBu, NF-κB PPTs, HLA-B-associated transcript 3 (BAT3) PPTs, NOX2 inhibitors, and nuclear proteins such as mixed-lineage leukemia protein-5 (MLL5) PPTs (e.g., splice variants thereof that induce IR(s)) or PPTs that block proliferating cell nuclear antigen (PCNA).

In aspects, CEPs comprise ITIM(s) (aka, ITIPM(s)) that modulate a transcription factor involved in IRs/CEs against CDAs, a protein that modulates a transcription factor, an internal portion or internally present oncoprotein, or a combination thereof. Examples of such include modulators of the human murine double minute 2 (MDM2) oncoprotein/p53 pathway or a MDMX/p53 pathway (see, e.g., Midgley C A et al. Oncogene. 2000; 19(19):2312-2323 and Phan J et al. J Biol Chem. 2010; 285(3):2174-2183). Additional transcription factors (“TFs”) involved in IRs include E2A, Pax5, EBF, PU.1, Ikaros, GATA3, Th-POK, Tbet, Bcl6, NF-κB, STATs, and IRFs. EPs can include modulators of such TFs that induce IRs. E74-like ETS transcription factors (e.g., ELF-1 and ELF-4) (SFE Seifert et al. PLoS Pathog. 2019; 15(11): e1007634). EPs can, e.g., comprise ELF PPTs, FFs, or FVs. In aspects, EPs include one or more ITIMPPTs of the interferon-regulatory factor (IRF) proteins (IRF) family of TFs or FFs or FVs thereof (described in, e.g., Yanai H et al. Oncoimmunology. 2012; 1(8):1376-1386). Additional TFs involved in IRs include PPTs of the nuclear factor of activated T-cells (NFAT) family, Activator Protein-1 (AP-1) (inhibitors of AP-1 are EPs in one aspect), and NF-dB. In aspects, an ITIM is a PPT involved in ubiquitinylating PPTs, such as a ubiquitin ligase. An example of such an ITIM is tripartite motif containing-21 (TRIM21).

In other aspects, ITIM(s) comprise MAPK pathway PPTs or modulators thereof, e.g., p38 MAPK PPT(s). In aspects, ITI(s) comprise ERK1/2 pathway PPT(s) or modulator(s) thereof (e.g., inhibitors of such a pathway CB enhanced Th1 IR(s)/reduced Th2 responses). In aspects, OSMGAOA ITIMs in CEPs are non-Ab PPTs. In aspects, OSMGAOA ITIMs are Abs or Ab FPs. In aspects, OSMGAOA Ab CEPs are intrabodies (Abs targeting intracellular targets in cell(s) of expression (COE)).

In aspects, OSMGAOA PCMs are innate trained immunity cell (ITIC) internal target modulator(s) (“ITICITMs”). ITICITMs modulate activity of targets primarily, generally only, substantially only, or only expressed in innate trained immunity cells (ITICs); modulate IRs primarily, generally only, substantially only, or only in ITICS; or both. In aspects, ITICITM(s) do not include ITS(s), e.g., PTPS(s), ERTPS(s), TF(s), etc. In aspects, ITICITMs DOS bind PPTs or portions of PPT(s) present in the internal portions of ITICs, rather than extracellular targets (e.g., internal portions of CPCRs, cytosolic PPTs, or organelle associated PPTs). ITICITMs typically also are PCMs. In aspects, ITICITMs in CEPs DOS induce IRs in COE, in other cells, or both.

In aspects, ITICITM(s) comprise STING (simulator of interferon genes) modulator(s) (e.g., STING agonists), STING PPTs, or both. STING modulators, related techniques, and applicable principles are described in, e.g., US20180085432 and WO202002874. In aspects, a STING modulator(s) DOS exhibit IRs in T-cells, NKCs, monocytes, or combinations. In aspects, STING modulator(s) DOS primarily exhibits IRs in such cells. In aspects, STING modulator(s) also exhibit DOS IR(s) in lung, ovary, heart, smooth muscle, retina, bone marrow, or vagina cells. In aspects, a STING modulator EP comprises a mitochondrial ITS (SFE Diekert K et al. Proc Natl Acad Sci USA. 1999; 96(21):11752-11757; Del et al. Mol Ther. 2003; 7(6):720-730; and Yogev 0 et al. Biochim Biophys Acta. 2011; 1808(3):1012-1020), a lysosome TS, e.g., of lamp-1, LIMPII, or MHCII invariant chain (SFE Starodubova E S, et al. Acta Naturae. 2014; 6(1):61-68; Behnke J et al. FEBS Lett. 2011; 585(19):2951-2957; and Schrader-Fischer G, et al. 1997; 68(4):1571-1580) an ERTPS, or CT. In aspects, a STING modulator EP is a cGAS PPT (e.g., Uniprot Q8N884, I3LM39, or E1BGN7, a homolog, a chimera, an FF, or FV of any such PPT); a TMEM203 PPT (or FF or FV) (see Yang et al. PNAS August 2019, 116 (33) 16479-16488). In aspects, CEPs comprise PPTs that block PPTs that block STING activity (e.g., an anti-DENV or anti-Zika virus NS2B3 PPT Ab).

In aspects, ITMs are factors in or modulators of factors in a checkpoint cascade, such as signaling mediators. E.g., CEPs can comprise PLCyl, PLCy2, and PI3K PPTs, which in aspects are signaling mediators of EAT-2/CRACC checkpoint pathway(s), e.g., in NKCs.

In aspects, OSMGAOA peptidic checkpoint modulator(s) (PCM(s)) in CEPs are co-stimulatory molecule(s) CSM(s). In aspects, OSMGAOA PCSMs are immune cell receptor ligand(s) (ICRL(s)) of co-stimulatory immune cell receptor(s) (ICR(s)). Examples of peptidic CSMs include 4-1BBL PPTs (e.g., a WT PPT, an FF, or a FV of either), GITRL PPTs, CD80 (B7.1) PPTs, CD28L PPTs, CD137L PPTs, ICOS ligand PPTs, CD86 (B7.2) PPTs, CD70 PPTs, H7-H7 PPTs, CD30 PPTs, CD40 PPTs, and HVEM PPTs. Examples of PCSMs also include activating modulators of GITR (CD357) (e.g., a GITRL PPT), CD28 (e.g., PD-L1 PPTs or B7-1 PPTs), or ICOS. In aspects, a PCM modulates a T-cell CSM CPCR. Examples of such PCMs include modulators of OX40 (CD134) (e.g., OX40L/CD252 PPTs), 4-1BB (CD137 (e.g., CD137L PPTs)), CD28, CD27, ICOS, and CD122. In aspects, a PCM modulates a B cell CSM CPCR (e.g., a CD40 modulator, a completement receptor modulator, a CR2 modulator (e.g., a CD19 or CD81 PPT), or a combination thereof.

In aspects, OSMGAOA PCMs in CEPs are peptidic CIs (PCIs). In aspects, PCM(s) exhibit DOS checkpoint inhibition effects in T-cells, NKCs, DCs, or combinations. In aspects, PCM(s) primarily, generally, or at least substantially only exhibit DOS checkpoint inhibition IRs in T-cells and NKCs. Examples of checkpoint inhibitors include CTLA-4 (CD152), PD-1/PD-L1 (CD274) or PD-L2 (CD273) checkpoint inhibitors. Other examples of checkpoint inhibitors include PCMs that inhibit a LAG-3 (CD223) or KIR (CD158) checkpoint, or that inhibit a TIM-3, TIGIT, Galectin, Poliovirus receptor (CD155), or 4-1BB (CD137) checkpoint pathway, or combinations. See, e.g., Chen L, Flies D B. Molecular mechanisms of T cell co-stimulation and co-inhibition [published correction appears in Nat Rev Immunol. 2013 July; 13(7):542]. Nat Rev Immunol. 2013; 13(4):227-242.

In aspects, gDP(s) in CEPs can act as checkpoint inhibitors, DOS inhibiting the HVEM/BTLA checkpoint pathway. In aspects, CEPs comprise both gDP CI(s) and NGDCI(s). In aspects, CEPs only comprise gDP CI(s). In aspects, the only type of PCMs in CEPs are gDPs. In aspects, CEPs comprise gDP CI(s) and PCSM(s) (e.g., 4-1BB pathway CSMs or GITR pathway CSMs). In aspects, gDPs of a CEP do not act as CIs in TRs (e.g., gDPs do not block HVEM in non-HVEM-expressing TRs, such as pigs). In aspects, the only EPs with CI function are non-gD Cis (NGDCIs) (e.g., LAG-3s, TIM-3s, or KIRs).

In aspects, OSMGAOA PCMs in CEPs are signal transducing adaptor protein(s) (STAP(s)). STAPs (a.k.a. “adaptor proteins” sometimes presented as “adapter” proteins) are PPTs that modulate signal transduction pathways, such as ITIM-mediated pathways (discussed above), ITAM pathways, or other pathways. STAPs are further described in US20200325182. In aspects, OSMGAOA of the PCMs of a CEP are STAP(s), STAP modulators, or both. In aspects, STAP(s)/STAP modulator(s) DOS modify checkpoint pathway(s) (i.e., CPSTAPs or CPSTAPMs). In aspects, PCMs can be classified as both (1) immune cell STAPs (ICSTAP(s)) due to primary expression in ICs and (2) CPSTAP(s). In aspects, a PCM is a modulator of an ICSTAP, CPSTAP, or both. In aspects, a CPSTAP activates IRs in ICs. In aspects, an ICSTAPM/CPSTAPM blocks/inhibits an inhibitory STAP or DOS modifies IR(s).

STAPs include adaptor protein complex 3 (AP-3), DAP10, DAP12, slp-76 family adaptor(s) (e.g., Slp-76), Nck, Itk, AP2 clathrin adaptor, the Crk adaptor, and the EAT-2 activator. Innate immune receptor-associated adaptors include MyD88 or TRIF. TNFa CPCRs are modulated by TRAF adaptors, which can be included in EPES(s). In aspects, OSMGAOA of STAP(s) in CEPs are transmembrane adaptor proteins (TRAPs), such as LAT, NTAL (non-T-cell activation linker), PAG (protein associated with glycosphingolipid-enriched microdomains) and LIME (LCK-interacting membrane protein), TRIM (TCR-interacting molecule), SIT (SH2-domain-containing protein tyrosine phosphatase (SHP2)-interacting transmembrane adaptor protein) and LAX (linker for activation of X cells, where X denotes an as yet unidentified cell). In aspects, a PCM comprises an inhibitor of an inhibitor TRAP. In aspects, a PCM comprises a stimulatory TRAP PPT or a modulator of a stimulatory TRAP PPT that DOS induces IRs. In aspects, a TRAP PPT is of or is related to a lipid raft-associated TRAP PPT or a TRAP modulator acts on such a TRAP PPT. In aspects, a TRAP PPT is a non-lipid raft associated TRAP or a modulator of such a TRAP. See, e.g., Hořejší, V et al. Nat Rev Immunol 4, 603-616 (2004). Additional STAPs include the adhesion and degranulation-promoting adapter protein (ADAP), CG-NAP/kinase, the Grb2-related adaptor downstream of Shc (Gads), Shc, zeta (ξ)-chain associated protein of 70 kDal (Zap-70) and the linker for activation of T cells (LAT).

In aspects, a STAP EP modulates IRs in multiple IC types, interacts with multiple PPTs involved in IRs, or both. The adaptor SLP-76, for example, modulates both NK cell activating receptor functions and NKT cell functions, such as selection, differentiation, and activation and SLP-76 also modulates Vav family proteins in NK activating signal transduction. Similarly, SH2 domain-containing inositol 5′-phosphatase (SHIP) which both regulates the P13K pathway and CD4 signal pathways is modulated by adaptors Dok-1/2 (Waterman P M et al. Immunol Lett. 2012; 143(1):122-130). Like SLP-76 adaptors, Crk STAPs (Crk, CrkII, Crk I, v-Crk, or CrkL-SFE Birge, R. B et al. Cell Commun Signal 7, 13 (2009) modulate numerous PPTs. ICSTAPs FcR-γ & DAP12 modulate activity of a number of ICRs, in some cases inhibiting activity (e.g., in NKp44) and in other activating activity (e.g., Dectin-2 & TREM-2) (SFE Hamerman J A et al. Immunol Rev. 2009; 232(1):42-58). In aspects, CEPs comprise TRAF adaptor STAP(s). In aspects, STAP(s) comprise PPT(s) that block c-Cb1, Cb1-b, or both.

In aspects, CEPs comprise STAPs that modulate ICRs of the signaling lymphocyte activation molecule (SLAM) family. In aspects, OSMGAOA of PCPMs in CEPs are adaptor(s) of a SLAM receptor. In aspects, OSMGAOA SLAM receptor adaptors (SLAMRAs) contain SH-2 motif(s), contain cytoplasmic ITIMs, are tyrosine phosphorylated PPTs, or exhibit a combination thereof. In aspects, PCMs are STAP(s) that modulate a SLAM receptor that is regularly expressed in DCs (e.g., SLAM1, SLAM2, SLAMF5, or SLAMF7). In aspects, CEP PCMs are STAP(s) that DOS modulate SLAMF5/CD86, SLAMF7/CRACC, or both. In aspects, such STAPs primarily induce IRs. WT SLAM-modulating STAPs include SAP (SLAM-associated protein-also named SH2D1A), Ewing's sarcoma-associated transcript-2 (EAT-2; also named SH2D1B1) & EAT-2-related transducer (ERT; also named SH2D1B2) (“SAP family adaptors” or SAPFAs). In aspects, PCMs comprise WT SAPFAs or SAPFA FPs comprising WT SAPFA AARSs. In aspects, PCMs comprise FFs of SAPFAs in NFPs or FPs. In aspects, PCMs comprise FVs of SAPFAs in NFPs or FPs. In aspects, OSMGAOA SAPFAs in CEPs are in FPs. In aspects, OSMGAOA SAPFAs in CEPs are contained in gDPFPs (gdFPs). In aspects, OSMGAOA SAPFAs in CEPs are EAT-2 PPTs (e.g., EL WT EAT-2s, FFs thereof, or FVs). In aspects, EAT-2 PPTs are hEAT-2 PPTs (EL WT hEAT-2, FFs thereof, or FVs thereof).

In aspects, PCMs are described by the checkpoint pathway cell receptor(s) (CPCR(s)) in a PCMs relevant checkpoint pathway. In aspects, PCMs are ligands of CPCRs. In aspects, PCMs are CPCRs (e.g., CPCRs that bind other CPCRs, CPCRs that are self-ligands, such as self-ligand SLAM CPCRs, or soluble forms of CPCRs, either WT soluble forms or soluble FFs/FVs of non-soluble CPCRs). In aspects, PCMs modulate B7/CD28 family/superfamily CPCR pathway(s). In aspects, PCMs modulate immunoglobulin CPCR pathway(s). In aspects, PCMs modulate TNF superfamily CPCR pathways. In aspects, CEPs comprise PCMs that modulate checkpoint pathways involving CPCRs of two of these families/superfamilies (“families”). In aspects, CEPs comprise PCMs that modulate checkpoint pathways involving CPCRs of all three families. In aspects, CEPs comprise two or more PCMs that modulate two or more CPCRs in at least one of these families (e.g., two or more PCMs that modulate TNF superfamily CPCRs or 2+ PCMs that modulate B7 family CPCR pathways and 2+ PCMs that modulate immunoglobulin superfamily CPCR pathways).

In aspects, PCMs include modulators of a B7 CPCR pathway (e.g., CD28 and CTLA-4 and their ligands B7.1 (CD80) and B7.2 (CD86); ICOS and ICOS ligand (ICOSL); and the coinhibitory receptor Programmed Death Receptor 1 (PD-1) and its ligands PD-L1 and PD-L2; the CD28 homolog member B and T lymphocyte attenuator (BTLA); and 2 B7 homologs B7-H3 and B7-H4 (B7×, B7S1)). Additional members of this PPT family include ILDR2 (Hecht I et al. 2018; 200(6):2025-2037). In aspects, B7 CPCR pathway modulating PCM(s) modulates a costimulating pathway (e.g., the CD86/CD28 pathway), an inhibitory pathway (e.g., CD80/CTLA4), or both. In aspects, PCMs block activity of inhibitory B7 CPCR pathways (e.g., B7-H3). Other PPTs in the B7 family CPCR that can be or can be modulated by PCMs include, e.g., B7-H6, B7-H7/HHLA2, PDCD6, TMIGD2/CD28H, VISTA/B7-H5/PD-1H, Nkp30, TMIGD2, HHLA2, CD276, B7-DC, B7-H5, and CD272. In aspects, B7/CD28-related PCM(s) in CEPs DOS induce T cell activation, T cell proliferation, IC cytokine production, promotion of TC survival, B cell activation, TH cell differentiation, IC cytotoxicity (e.g., NKC-mediated cytotoxicity, CTL-mediated cytotoxicity, or both), or combinations of any/all thereof.

In aspects, PCMs comprise a member or modulator of a PD-1 checkpoint pathway. In aspects, PCMs comprise a PD-L1-binding PPT, a PD-L2 binding PPT, or both. In aspects, PCMs comprise inhibitors of a PD-1 pathway. In aspects, PCMs comprise PD-L1 Abs or Ab FPs. In aspects, PCMs comprise non-Ab PD-L1 binding PPTs. In aspects, PCMs comprise multimeric non-Ab PD-L1 trap proteins. In aspects, a PCM comprises an FF of a PD-L1, such as a PD-L1 extracellular binding domain (or a FV thereof). In aspects, PCMs comprise a PD-1 PPT or another PD-1 modulator, such as a PD-1 extracellular binding domain (or a FV thereof). In aspects, subjects/TRs are pigs and CEPs comprise a PD-1 or PD-L1 PPT or modulator that is expressed in pigs, such as an extracellular domain of a PD-1 or PD-L1 PPT.

In aspects, a PCM is a member or modulator of a CTLA-4 checkpoint pathway. In aspects, PCM(s) block CTLA-4 to CD80 (B7-1) and/or CD86 (B7-2). In aspects, the CTLA-4 AARS or PPT comprises at least a portion of an anti-CTLA-4 Ab, such as ipilimumab or ticilimumab. Associated IR(s) are described in, e.g., US20200325182. In aspects, CEPs comprise PCM(s) that modulate a V-domain Ig suppressor of T cell activation (VISTA) pathway (sometimes aka B7-H5, though HHLA2 also has been described as B7-H5). In aspects, PCM(s) block VISTA pathway(s). In aspects, such PCM(s) comprise anti-VISTA Abs or Ab FPs. Anti-VISTA Abs are known in the art (e.g., NCT02671955) (see also Deng J et al. J Immunother Cancer. 2016; 4:86). In aspects, PCMs modulate Butyrophilin family CPCR(s) (sometimes aka, B7 family CPCRs), e.g., BTN1A1/Butyrophilin, BTN2A1, BTN2A2/Butyrophilin 2A2, BTN3A1/2, BTN3A2, BTN3A3, BTNL2/Butyrophilin-like 2, BTNL3, BTNL4, BTNL6, BTNL8, BTNL9, BTNL10, and CD277/BTN3A1. In aspects, PCM(s) block the action of inhibitory Butyrophilin pathway(s) (e.g., a BTN1A1, BTN2A2, or BTNL2). In aspects, PCM(s) activate stimulating Butyrophilin pathway(s). In aspects, PCMs comprise a BTN3A PPT. The biology of such PCMs is discussed in, e.g., Malinowska M et al. Cent Eur J Immunol. 2017; 42(4):399-403. doi:10.5114/ceji.2017.72806. In aspects, PCMs comprise ILT/CD85-ILIR (Inhibitory leukocyte immunoglobulin-like receptor A/B) CPCR modulator(s) (also described as or overlapping with Leukocyte immunoglobin-like receptor (LIR) CPCR pathways). In aspects, such PCMs DOS induce T-cell IRs. Examples of such CPCRs and associated ligands include LILRA1/CD85i, LILRA2 (ILT1)/CD85h, LILRA3 (ILT6)/CD85e, LILRA5/CD85f, LILRA6/CD85b, LILRA4/CD85g/ILT7, LILRB1/CD85j/ILT2, LILRB2/CD85d/ILT4, LILRB3/CD85a/ILT5, and LILRB4/CD85k/ILT3. Modulators of such CPCRs include Angiopoietin-like (ANGPTL) proteins (e.g., human Angiopoietin-1, Angiopoietin-2, and Angiopoietin-4).

In aspects, CEPs comprise PCM(s) that are of, are related to, or are modulators of immunoglobulin CPCR superfamily pathway(s). Examples of such CPCRs/modulators include CEACAMI (self-ligand), CD96/CD155, etc. In aspects, such PCMs comprise ligands of an activating immunoglobulin CPCR superfamily receptor, e.g., a Nectin-2/CD112 PPT or a CD48/BCM1 PPT. In aspects, PCMs do not comprise any Nectin family PPTs that are bound by gDPs in CEPs. E.g., in an aspect, CEPs comprise a Nectin-2/CD112R PPT PCM and no gDPs that exhibit significant binding to Nectin-2. In aspects, PCMs do not comprise Nectin family PPTs. In aspects, PCMs do not comprise PCMs that modulate both activating & inhibiting CPCR pathways.

In aspects, OSMGAOA PCM(s) in a CEP modulate a LAIR Family CPCR checkpoint pathway. Examples of such CPCRs include LAIR1, LAIR2, CD96, CD155/PVR, CRTAM, DNAM⁻¹/CD226, PVRIG, and TIGIT. Nectin and Nectin-like Ligand/Receptor Co-Signaling Molecules, Nectin-2/CD112, and Nectin-3 also have been associated with the LAIR family and checkpoint pathways (e.g., Nectin-2/PVR/CD112R, is a ligand for TIGIT, a well-established CI). In aspects, PCM(s) modulate the CD112/CD112R (PVR/Nectin-2) CPCR pathway. In aspects, PCM(s) block CD112R-CD112 interactions. In aspects, such PCM(s) are Abs or Ab FPs. In aspects, such PCMs are non-Ab FPs. Non-Ab FP CD112R CIs can comprise soluble CD112/Nectin-2/PVRL2 PPTs. In aspects, non-Ab FP CD112 PCMs are CD112R trap PPTs. In aspects, PCM modulators of the CD112R/CD112 pathway DOS enhance IC IFN-γ- or IL-17 production. In aspects, CEPs comprising such PCMs exhibit DOS recruitment/concentration or IRs mediated by DCs, monocytes, or both. In aspects, PCMs further comprise AARS(s) that block CD112R, TIGIT, or both. In aspects, CEPs comprise PCMs that block both CD112R and TIGIT. Aspects of such CIs that can be adapted to these aspects are discussed in, e.g., Zhu Y, Paniccia A, Schulick A C, et al. J Exp Med. 2016; 213(2):167-176 and US20200325182. A CD112R epitope AVLHPERGIRQWAPARQ (SEQ ID NO:732) can be bound by CD112R-modulating PCMs, see, e.g., US20170240613A1.

In aspects, CEPs comprise PCMs that modulate a TIGIT (T cell immunoreceptor with immunoglobulin and ITIM domains) pathway or a CD96 pathway. In aspects, PCM(s) block TIGIT pathway(s). In aspects, PCM(s) activate CD96 pathway(s). In aspects, PCMs DOS inhibit CD155 binding to TIGIT. In aspects, such PCMs can DOS exhibit enhanced CD155 (PVR) binding to activating CPCR CD226. In aspects, PCMs block CD155/PVR interactions. In aspects, PCMs block all three of PVR, TIGIT, and CD96 from interactions. In aspects, PCMs that block TIGIT pathway(s) exhibit DOS IRs in NKCs, T-cells (e.g., CTLs), or both. In aspects, such PCMS DOS reduce T-cell exhaustion. In aspects, such PCMs DOS induce enhanced cytokine production (e.g., IFNg or TNF-alpha). In aspects, such CEPs comprise anti-cancer Ags. In aspects, PCM(s) comprise CD96 PPTs or activators of a CD96 pathway. In aspects, such PCMs DOS enhance T cell IRs (e.g., T cell differentiation or cytokine levels). In aspects, PCM(s) comprise CD226 activating PCMs. In aspects, CEPs comprise modulators of a TIM CPCR pathway (e.g., TIM⁻¹/KIM⁻¹/HAVCR, TIM-3, Galectin-9, HMGB1, CEACAM⁻¹, TIM-2, H-ferretin, Semaphorin 4A, and TIM-4. In aspects, CEPs comprise PCM(s) that modulate the TIM-3 3 (T-cell immunoglobulin and mucin domain 3) pathway. In aspects, PCM(s) comprise TIM-3 ligands. In aspects, TIM-3 ligand(s) comprise TIM-3 Abs or Ab FPs (such Abs are known—e.g., Sym023, Cobolimab, LY3321367, BGB-A425, and MBG453). In aspects, TIM-3 ligand(s) comprise TIM-binding traps. In aspects, TIM-3 ligands comprise Galectin-9, HMGB1, or CEACAM⁻¹ PPTs.

In aspects, PCM(s) comprise modulator(s) of a SIRP CPCR family receptor pathway. Examples of such CPCRs include signal regulatory protein-α (SIRPα or SIRPα-a.k.a. CD172a). Other SIRP CPCRs include SIRP beta1, SIRP beta2, and SIRP gamma. In aspects, PCM(s) inhibit a SIRP CPCR, e.g., binding to the CPCR, its ligand (CD47, another CPCR), or both. Such PCM(s) can be anti-SIRP or anti-CD-47 Abs, Ab FFs, or Ab FPs. (e.g., Magrolimab (Hu5F9-G4) is a known anti-CD47 Ab). Such PCMs can be SIRP-binding trap PPTs. In aspects, such PCMs DOS enhance NKC activity. Such CPCRs are described in, e.g., Li C W et al. Front Med. 2018; 12(4):473-480.

In aspects, CEPs comprise modulator(s) of KIR (killer cell immunoglobulin-like receptors) Family CPCR(s). KIR CPCRs include inhibitory KIR2DL1, KIR2DL2, & KIR2DL3 CPCRs. Other KIRs are activating CPCRs. In aspects, PCM(s) are modulator(s) of inhibitor KIR(s). In aspects, PCM(s) are modulator(s) of activating KIR(s). In aspects, PCMs comprise anti-KIR Abs, Ab fragments, or Ab FPs, anti-KIR-ligand Abs, Ab fragments, or Ab FPs, or both. Examples of such Abs are described in, e.g., US20120208237, and include Lirilumab (1-7F9, IPH2101)).

In aspects, CEPs comprise PCM(s) that modulate a natural cytotoxicity receptor (NCR) pathway. NCRs are classified as NKp46, NKp44, or NKp30 CPCRs, but isoforms of such CPCR(s) can exist with different properties. E.g., NKp30 isoforms NKp30a and NKp30b evoke NKC activation, the NKp30c isoform was shown to elicit secretion of the immunosuppressive cytokine, IL-10 from NKCs. In aspects, NCRs comprise both activating and inhibiting CPCRs. In aspects, NCR(s) comprise NKp44-1 AARS(s). In aspects, PCM(s) block inhibitor NCR(s), ligand(s) of such NCRs, or both. Ligands of inhibitory NCRs are known and include B7-H6 PPTs, Nidogen-1 PPTs, and Galectin-3 PPTs. In aspects, PCM(s) comprise Abs or Ab FPs against such ligand, inhibitory NCRs, or both. In aspects, PCM(s) comprise ligands of activating NCRs, such as PDGF-DD PPTs or agonistic Abs against such CPCRs. DCA-associated PPTs, such as influenza HA/HN PPTs modulate NCRs and can be used as PCMs, Ags, or both. Aspects of such ligands and modulation of NCRs is known and such knowledge can be applied to aspects (SFE Barrow A D et al. Front Immunol. 2019; 10:909. and Kruse P H et al. Immunol Cell Biol. 2014; 92(3):221-229). In aspects, PCMs include an ITICSTAP that promotes activation of an activating NCR, e.g., a DAP12 PPT. Intracellular ligands modified by NCRs include HLA-B-associated transcript 3 (BAT3), proliferating cell nuclear antigen (PCNA), internal Ag(s) such as HIV gp41, mixed-lineage leukemia protein-5 (MLL5), and NKp44L.

In aspects, CEPs comprise PCM(s) that modulate a pathway of a signaling lymphocyte activation molecule (SLAM) CPCR family. Examples of SLAM CPCRs include SLAM/SLAMF1, CD48/SLAMF2, CD58/LFA-3, CD229/SLAMF3, 2B4/CD244/SLAMF4, CD84/SLAMF5, NTB-A/SLAMF6, CRACC/SLAMF7, BLAME/SLAMF8, CD2F-10/SLAMF9, and CD2. Additional SLAMF members & related PPTs are discussed elsewhere and are described in, e.g., O'Connell P et al. Vaccines (Basel). 2019; 7(4):184. Most SLAM ICRs (SLAMF1, 3, 5, 6, & 7) are self-ligands (SLAMF2 and SLAMF4 are ligands for each other). In aspects, OSMGAOA of PCM(s) in CEPs are adaptor(s) of a SLAM receptor. In aspects, OSMGAOA SLAM receptor adaptors (SLAMRAs) contain SH-2 motif(s), contain cytoplasmic ITIMs, are tyrosine phosphorylated PPTs, or exhibit combinations. In aspects, such CEPs induce DOS IRs in NKCs, CD8+ T cells, NKT cells, γδ T cells, monocytes, basophils, eosinophils, DCs, thymocytes, basophils, macrophages, B cells, or mast cells, or combinations.

In aspects, PCM(s) comprise a SLAM4 pathway PCM and one or more anti-viral Ags. In aspects, PCM(s) are SLAMF5 modulators. In aspects, such PCM(s) DOS induce enhanced IL-10, IL-23, or IL-12 expression. In aspects comprising SLAM4/SLAM5 pathway PCMs, IRs can comprise DOS reduction of T cell exhaustion. In aspects, CEPs comprising viral Ag(s) also comprise PCMs that block the SLAMF1 pathway, SLAMF3 pathway, or both. In aspects, any of such CEPs DOS enhance IFNg expression, DC activity (e.g., IL-12 or IL-8 secretion), B cell activity, NKC cytotoxicity, T cell activity, etc. In aspects, CEPs comprise PCMs that DOS modulate ≥2 SLAM CPCR pathways (e.g., SLAMF7 and SLAMF5).

In aspects, PCM(s) include STAP(s) that modulate a SLAM receptor that is regularly expressed in DCs (SLAMF1, SLAMF2, SLAMF3, SLAMF5, SLAMF6, SLAMF7, & SLAMF9). In aspects, PCMs are STPAP(s) that modulate SLAM1, SLAM2, SLAMF5, SLAMF7, SLAMF8, or SLAMF9, which are associated with higher levels in DCs. In aspects, PCM(s) are STAP(s) that modulate SLAM(s) in NKC(s) (e.g., SLAMF4) or both NKCs & DCs (SLAMF2, SLAMF3, SLAMF5, SLAMF7, or SLAMF8). SLAM-associated STAP PCMs are discussed elsewhere here and, e.g., Veillette A. Immunol Rev. 2006; 214:22-34; Cruz-Munoz, M., et al. Nat Immunol 10, 297-305 (2009); and Tassi I et al. J Immunol Dec. 15, 2005, 175 (12) 7996-8002. In aspects, PCM(s) comprise both SLAM CPCR-modulating STAP(s) (e.g., SAP PPTs or EAT-2 PPTs) and SLAM CPCR extracellular ligand(s) (e.g., CRACC PPTs). In aspects, OSMGAOA SLAM pathway modulator(s) in CEPs are SLAM CPCR STAP(s), e.g., EAT-2 PPTs. In aspects, CEPs comprise PCMs that block SLAMF7 inhibition of ICs, such as TCs (e.g., PPTs that block SLAMF7 inhibitory pathway modulators that are specific to T-cells and not expressed in significant amounts in DCs). In aspects, PCM(s) comprise modulators of SLAM STAP(s), such as PPTs that bind and block the functioning of EAT-2 PPTs, SAP PPTs, or both.

In aspects, SLAM pathway PCM(s) comprise anti-SLAM or anti-SLAM ligand Abs or Ab FPs. In aspects, Ab FPs comprise Fc domains. An example of such an Ab FP is a CRACC-Fc FP described in references cited herein. Anti-SLAM Abs that induce IRs are known and include, e.g., Elotuzumab (SFE Pazina T et al. Oncoimmunology. 2017; 6(9): e1339853. Published 2017 Jun. 16; Aldhamen Y A et al. Vaccine. 2016; 34(27):3109-3118; and Malaer J D et al Am J Cancer Res. 2017; 7(8):1637-1641). In aspects, SLAM pathway PCM(s) are non-Ab PPTs. In aspects, SL AM pathway PCM(s) are multimeric trap proteins (e.g., a SLAM7 trap, a SLAM4 trap, or a SLAM5 trap).

In aspects, PCMs comprise LAG-3 CMs. Lymphocyte activation gene 3 (LAG-3; also known as CD223) modulates regulatory T cells (TRegs) and suppresses CD8 TCs. In aspects, PCMs block one or both LAG-3 effects. Such a PCM can bind and block LAG-3 or a LAG-3 ligand (e.g., Galectin-3, fibrinogen-like protein 1 (FGL1), or both). In aspects, PCMs also can modulate PPTs in a pathway that leads to DOS enhanced expression of an inhibitory checkpoint PPT, such as LAG-3 (this approach can be applied to any of the inhibitory checkpoint PPTs described here). In aspects, EP(s) comprise both a LAG-3 and a PD-1 or PDL-1 CI AARS or PPT. LAG-3 Abs that can be incorporated in EP(s) in whole/part are described in WO2010019570; WO2008132601; and WO2004078928. Eftilagimod alpha (IMP321), is an example of a LAG3 Ab FP adaptable to aspects. In aspects, EP(s) induce NKC cytokine production, sustain NKC activation, or both. In aspects, blocking the LAG-3 checkpoint pathway with PCM(s) DOS induces T-cell IRs. In aspects, CEPs comprise both LAG-3 blocking PCM(s) and PD-1 pathway blocking PCM(s). In aspects, such CEPs result in DOS reduction of T cell exhaustion. In aspects, CEPs comprise LAG-3 blocking PCM(s) and TIM-3 blocking PCM(s), B7-H1 blocking PCM(s), or both. In aspects, LAG-3 pathway blocking PCMs comprise PCMs that bind/block ≥1 or ≥2 LAG-3 ligands, bind and block LAG-3, or both.

In aspects, CEPs comprise PCM(s) that modulate a CD200R CPCR pathway. In aspects, PCM(s) block a CD200R pathway. In aspects, such PCMs comprise anti-CD200R Abs or Ab FPs, anti-CD200 (OX2) Abs or Ab FPs, or both. Anti-CD200 Abs are known in the art (SFE Mahadevan, D et al. j. immunotherapy cancer 7, 227 (2019)). In aspects, CEPs comprising such PCM(s) induce IRs comprising DOS enhanced IL-2 expression, enhanced IFNg expression or both. In aspects, delivery of such CEPs induces DOS IRs in T-cells, NK cells, B cells, or combinations. In aspects, CEPs comprise PCM(s) that are CD200AR modulators. In aspects, such PCM(s) activate a CD200AR CPCR. In aspects, such a PCM is a ligand of CD200AR, such as a CD200AR-L. See, e.g., Olin M R et al. Cancers (Basel). 2019; 11(2):137 and Xiong Z et al. Clin Cancer Res. 2020; 26(1):232-241). In aspects, such a PCM is a multimeric trap.

In aspects, PCM(s) modulate Sialic acid-binding Immunoglobulin-like lectins (Siglec) pathway(s). Such CPCRs include Siglec-2, Siglec-3, Siglec-5, Siglec-6, SIglec-7, Siglec-8, Siglec-9, Siglec-10, and Siglec-11. In aspects, PCM(s) block Siglec-7 and Siglec-9 CPCR pathway(s). In such aspects, CEPs induce DOS NKC IR(s). In aspects PCM(s) block Siglec-2 pathway(s) or Siglec-3 pathway(s). In aspects such PCM(s) comprise anti-Siglec Abs/Ab FPs. Such Ab PPTs are known (e.g., Inotuzumab ozogamicin & Gemtuzumab ozogamicin).

In aspects, PCM(s) comprise modulators of TNF Superfamily CPCR pathway(s). In aspects, PCM(s) modulate death-domain-containing CPCR pathway(s). In aspects, PCM(s) modulate TNF family decoy receptor CPCR pathway(s). In aspects, PCM(s) modulate TNF receptor-associated factor (TRAF) binding receptor pathway(s). In aspects, PCM(s) comprise TRAF STAP(s) or PPTs that modulate TRAF signaling.

In aspects, PCM(s) comprise PPTs of or that modulate 4-1BB/TNFRSF9/CD137, 4-1BB Ligand/TNFSF9, BAFF/BLyS/TNFSF13B, BAFFR/TNFRSF13C, CD27/TNFRSF7, CD27 Ligand/TNFSF7, CD30/TNFRSF8, CD30 Ligand/TNFSF8, OX-40/TNFRSF4, OX40 Ligand/TNFSF4, glucocorticoid-induced TNFR-related protein (GITR, TNFRSF18), GITR Ligand/TNFSF18, CD27, CD40/TNFRSF5, CD40 Ligand/TNFSF5, DR3/TNFRSF25, TLIA/TNFSF15, HVEM/TNFRSF14, LIGHT/TNFSF14, Lymphotoxin-alpha/TNF-beta, RELT/TNFRSF19L, TACI/TNFRSF13B, TL1A/TNFSF15, or TNF RII/TNFRSF1B. BTLA/CD272 is sometimes associated with the TNF CDCR Superfamily and sometimes associated with the B7/CD28 family. In aspects, PCM(s) comprise GITRL PPTs or GITR activators and such PCM(s) DOS induce T cell effector activity or T cell proliferation, promote T cell survival, or suppress TReg activity. In aspects, PCMs comprise a CD70 PPT or an activator of CD27. In aspects, PCMs DOS induce NKC survival or activation, induce T cell memory, or enhance B cell IR(s). In aspects, PCM(s) include CD154 PPTs or activators of CD40 CPCR(s), and such PCMs induce IR(s) in B cells. In aspects, PCM(s) include 4-1BBL PPTs or 4-1BB activators and such PCM(s) DOS induce IRs in NKCs, T-cells, or both (e.g., activation, survival, or effector functions). PMCs are provided in Claus C et al. Sci Transl Med. 2019; 11(496): eaav5989; Li Y et al. Cell Rep. 2018; 25(4):909-920.e4; and Vinay D S, Kwon B S. Immunotherapy of cancer with 4-1BB. Mol Cancer Ther. 2012; 11(5):1062-1070).

In aspects, PCM(s) in CEPs modulate an OX40 pathway. In aspects, PCM(s) are OX40 agonists/activators. In aspects, such PCM(s) induce IR(s) comprising CD4 and CD8 T-cell priming, proliferation, and function; reduction of TReg inhibition of CD8 T cells; neutrophil activation or survival; or combinations. In aspects, such PCMs comprise agonist Ab/Ab FPs. Agonist OX40 Abs are known in the art (OX86, see Jin H et al. JCI Insight. 2019; 4(21): e129736). In aspects, such PCMs comprise OX40L PPTs (e.g., WT OX40L PPTs, FFs, or FVs). In aspects, such PPTs are OX40 trap PPTs. In aspects, such PCMs DOS induce enhanced immunological memory.

In aspects, PCM(s) modulate HVEM/BTLA/Light pathway(s). In aspects, as discussed elsewhere, gDPs bind HVEM. In such aspects, PCM(s) can or cannot also include HVEM/BTLA/LIGHT modulating PCMs. In aspects, such PCM(s) are not included in CEPs. In aspects, CEPs do not comprise HVEM-binding gDPs, but comprise HVEM/BTLA/Light pathway modulating PCM(s). Such PCMs are known in the art (SFE WO2010006071A1). Anti-HVEM Abs are described in, e.g., Heo S K et al. J Leukoc Biol. 2006; 79(2):330-338. HVEM fragment peptides that block HVEM pathway(s) that can be adapted for use as PCMs are described in Spodzieja M et al. PLoS One. 2017; 12(6): e0179201.

In aspects, PCM(s) modulate C Lectin Family CPCR pathway(s). In aspects, PCM(s) modulate dectin-1 cluster CPCR(s) (e.g., dectin-1, CLEC-1, CLEC-2, LOX-1, CLEC12b, CLEC9a, or combinations). In aspects, PCM(s) modulate dectin-2 cluster CPCR(s) (e.g., dectin-2, blood DC antigen 2 (BDCA-2), DC immunoactivating receptor (DCAR), DC immunoreceptor (DCIR), CTL superfamily 8 (CLECSF8), or MINCLE (CLEC4E)).

In aspects, PCM(s) include modulators of an NKG2A/CD94 pathway. In aspects, PCM(s) inhibit NKG2A/or a NKG2A-related pathway. In aspects, such PCM(s) comprise Ab(s)/Ab FPs (e.g., IPH2201-Monalizumab).

Examples of other CPCRs/pathways that can be modulated by PCM(s) and that are not readily classified with the above-described groups include CD7, CD160, CD300a/LMIR1, CD300d, CD3001h, DPPIV/CD26, EphB6, Integrin alpha 4 beta 1, Integrin alpha 4 beta 7/LPAM⁻¹, TSLPR, & inhibitory CPCR adenosine A2a receptor (and its ligands TDO or IDO1).

In aspects, PIMs comprise peptidic IMs that are not typically classified as CMs. Examples of such non-CM IM PPTs (NCMIMPs), include cytokines & alarmins. In aspects, CEPs, AACs, or CCCs comprise cytokine(s). E.g., in aspects CEP(s) comprise chemokine(s), interferon(s) (IFN(s)), interleukin(s) (ILs, e.g., IL-2), lymphokine(s), tumor necrosis factor(s) (TNFs), or CT. Typically, hormones and most growth factors are not considered cytokines. CEPs can comprise peptidic hormones, growth factors, or both that act as IMs and AACs/CCCs can comprise both peptidic and non-peptidic IM hormones or growth factors or hormones or growth factors. Characteristics of suitable cytokines are provided in US20200325182. Cytokine EP(s) or CCCs, etc., can comprise interleukins (e.g., IL-2, IL-7, and IL-12) and chemokines (e.g., CCL3, CCL26, and CXCL7), lymphokines (e.g., GM-CSF and IFN-γ (IFNg)), cellular immune response cytokines (type 1—e.g., TNFα, IFN-γ, etc.), and type 2 (e.g., TGF-β, IL-4, IL-10, IL-13, etc.). Cytokines can be level 1 (early) cytokines, level 2 (intermediate), or level 3 (late), cytokines. Cytokines include type 1 cytokines (IL-12, IL-23, IL-6 and IL-10), type 2 cytokines (TSLP, IL-25, IL-33 and IL-1α, type 2 cytokines (e.g., IFN-γ, IL-17, IL-22 for type 1 immune response, and IL-4, IL-5, IL-9, IL-13 and AREG).

In aspects, CEPs comprise ≥1 , ≥2, or ≥3 cytokine PPTs. In aspects, EP comprises 2+ cytokine AARSs or PPTs. In aspects, OSMGAOA of any cytokine AARSs in a CEP are contained in one or more gDAgFPs. In aspects, EP comprises only one cytokine PPT/AARS. In aspects, CEPs lack any cytokine PPTs/AARSs. Exemplary cytokine PPTs and AARSs that can be included in CEPs include IFNγ, IL-2, IL-4, IL-6, IL-7, IL-10, IL-12, IL14, IL-13, IL-15, IL17, IL-18, IL20, IL21, IL-23, IL-24, IL-27, IL-28a, IL-28b, IL-29, KGF, TNF, TGF, GMCSF, MCSF, OSM, IFNα (e.g., INFα2b), IFNβ, GM-CSF, CD40L, Flt3 ligand, stem cell factor, ancestim, and TNFα(SFE Dranoff, Nat Rev Cancer. 2004 January; 4(1):11-22 and Szlosarek, Novartis Found Symp. 2004; 256:227-37; discussion 237-40, 259-69). Suitable chemokine AARSs and PPTs in EPs can include Glu-Leu-Arg (ELR)-negative chemokines such as IP-10, MCP-3, MIG, and SDF-1 alpha from the human CXC and C—C(or CC) chemokine families, such as MIPalpha, MIPbeta, MCP-1, IL-8, GROalpha, and IP-10. CEPs also can comprise AARSs or PPTs that are chemokines, such as RANTES, MIP-1α, Flt-3L (U.S. Pat. Nos. 5,554,512; 5,843,423) and the like. In aspects, cytokine(s) in CEPs or ACCs/CCCs DOS induce NKC functions (such cytokines, e.g., IL2, IL12, IFNg, and IL21, are described in, e.g., Wu Y et al. Front Immunol. 2017; 8:930.

In aspects, CEPs comprise alarmin PPT(s), stressorin PPT(s), or both; modulators of alarmin/stressorin pathway(s) (e.g., of a receptor thereof), or combinations. In general, alarmins/stressorins are similar to cytokines and some cytokines are classified as alarmins (e.g., IL-1α, IL-33, and IL-16). Other peptidic alarmins include high-mobility group box 1 protein (HMGB1), and the Ca2+-binding S100 proteins. Aspects of alarmins are described in US20200325182. Alarmins include heat-shock proteins (HSPs) (described elsewhere), adenosine triphosphate (ATP), nucleosomes, and mitochondrial components. Stressorins and alarmins are further described in US20200325182. In aspects, PIMs include a modulator of an alarmin receptor/PPT. Examples of such PPTs include DEC-205, TLR5, and HSP70.

In aspects, CEPs include defensin(s). In aspects, CEPs comprise defensin(s) with direct anti-DCA activity, IC signaling, or both. Defensin PIMs can comprise defensin PPTs (WT, FF, or FV), β defensins (WT, FF, or FV), or both. In aspects, CEPs comprise R defensin PPTs/AARs and such CEPs DOS induce DC migration. In aspects, ≥1 defensin AARs are expressed as FPs in CEPs (e.g., gDAgFP(s) in CEPs can comprise defensin AARS(s)). Defensins are further described in, e.g., US20200325182.

Another type of PIM that can be included in CEPs are PPTs/AARSs that can be characterized as adjuvants. Expressible adjuvants are described in US20200325182. Examples of PIMs that can be considered CEP adjuvants include TLRs, RIG-1, and NOD-like receptors and modulators thereof. Other examples of PIM adjuvants that can be incorporated in CEPs include muramyl dipeptide, aminoalkyl glucosaminide 4-phosphates, such as RC529, and bacterial toxoids or toxin fragments. Other PIMs that can be in CEPs as AARSs or PPTs include cathelicidin and eosinophil-derived neurotoxin.

Heat shock proteins (HSPs) can be classified as PIMAs or with other types of PIMs (e.g., alarmin-associated PPTs). In cases HSP AAARs are components of Ag FPs (e.g., CAg FPs). In cases PIMs include HSP gp96/Ag(s) FP. An exemplary composition is HSP-protein complex 96 (HSPPC-96; vitespen). In aspects, a PIM comprises HSP65 PPTs/AARSs. In aspects, such AARSs are in Ag FPs. In cases such FPs comprise anti-pathogen or anti-cancer Ag(s), such as a HPV16 E7 Ag. PIMs can include chaperone proteins, such as gp96 (HSP90B1), which chaperones peptides generated by intracellular proteasome degradation into class I MHC; and (ii) can function as a danger-associated molecular pattern & activate DCs by binding to TLR-2 & TLR-4. Vabulas R M et al. J Biol Chem 2002; 277:20847-53.

In aspects, PIMs comprise an Fc receptor PPTs/AARSs, which can both be characterized as a component of an Ab FP or a PIM. In aspects, FPs comprising an Fc receptor AARS DOS induce ADCC in TRs.

In aspects, PIMs comprise PRRs or PRR modulators, e.g., Toll-like receptors (TLRs), nucleotide-binding oligomerization domain (NOD)-like receptors (NLRs) and RIG-I-like receptors (RLRs) and peptidic modulators thereof. In aspects, PIMs comprise flagellin PPTs/AARs.

PIMs can also comprise MHC molecules, fusion proteins comprising MHC molecules/sequences, or molecules that bind to MHC molecules. MHC II binding peptides are described in WO2012027365, WO2011031298, US20120070493, and US20110110965.

In aspects, PIMs can be PPTs that modulate complement activity, such as blocking a completement inhibitor, such as CD46 or CD55 or an agonist for a completement receptor, such as CD21, a ficolin, or a related receptor modulator such as a modulator of CD19 or CD22. In aspects, a PIM of a CEP comprises a pathogen-associated molecular pattern (PAMP)-related IM, such as a PAMP mimetic that is a PIM (e.g., a mimotope), a PRR modulator (e.g., a PRR agonist), or both. In aspects, a PIM comprises an AARS or PPT that is an opsonin or other macrophage function modulator such as CRP, SAA, or SAP, a collectin (e.g., surfactant protein-A), or a mannose-binding lectin (MBL) or Lipopolysaccharide-binding protein (LBP). In aspects, a PIM comprises a scavenger receptor related IM, such as an IM of CD36 or CD68. In aspects, a PIM of an EP is a RAGE-associated IM (e.g., a HMGB-1 AARS/PPT or other DAMP), which also can be identified as an ITIM.

Adjuvant AARSs/PPTs also can comprise chaperone(s), e.g., cytoplasmic chaperone PPT(s)/AARS(s), ER chaperone PPTs/AARSs, viral intercellular spreading protein(s), cytoplasmic translocation polypeptide domain of a pathogenic toxin, centrosome targeting AARS(s), lysosome-associated membrane protein type 1 or associated PPTs, or FFs or FVS thereof. PIM adjuvant(s) can comprise calreticulin (CRT), N-CRT, P-CRT, C-CRT, γ-tubulin, Sig/LAMP-1, VP22 or a functional homolog, FF, or FV of any thereof. As indicated here, any of the PIM adjuvants described herein can be incorporated into FPs with other aspects of CEPs or can be expressed as NFP EPs.

In aspects, CEPs comprise modulators of ICRs (ICRMs), ICRLs (ICRLMs), or both that are not characterized as CPCRs, but that induce DOS IRs. Examples of such ICRs include, e.g., Toll-like receptor (TLR) (e.g., TLR1, TLR2, TLR4, TLR5 or TLR6 or TLR3, TLR7, TLR8 and TLR9) and examples of such ICRLMs include peptidic modulators thereof. An example of such ICRLMs are LPS mimotopes that interact with TLR-4 and induce IR(s) (SFE Shanmugam A et al. PLoS One. 2012; 7(2): e30839). CEPs can comprise modulators or PPTs of leucine-rich repeat-containing receptors (NLRs), NOD-Like receptors, RIG-I-like receptors (RLRs), or AIM-2 like receptors.

In aspects, CEPs comprise immunosuppressive PIMs. In aspects, CEPs do not comprise any immunosuppressive PIMs. Immunosuppressors comprise compositions that reduce, inhibit, delay, diminish, or otherwise suppress (“suppress”) IR(s). Typically, in aspects where CEPs comprise immunosuppressive PIM(s), the immunosuppressor(s) suppresses only certain IRs and does not DOS inhibit OSMOA IRs, does not DOS inhibit OSMOA CE(s)/IR(s). E.g., an immunosuppressor may inhibit IRs associated with cytokine overproduction (e.g., TH2 cytokine overproduction), so as to avoid a cytokine storm (cytokine release syndrome), while not inhibiting TH1 IR(s).

In aspects, CEPs comprise activating PI3K(s), such as the p110δ isoform (p110δ -PI3K), Class I PI3K. In aspects, CEPs comprise mammalian target of rapamycin (mTOR) PPTs. In aspects, CEPs comprise AKT PPTs. In aspects, CEP(s) comprise inhibitors of PTEN, SHIP, or both. In aspects, CEP(s) comprise VAV PPTs, e.g., Vav1, Vav2, or Vav 3 PPTs. In aspects, CEP(s) comprise PPTs that block/inhibits Bruton's tyrosine kinase (BTK); growth arrest specific 6 (Gas6); receptor-interacting serine/threonine kinase 1 (RIPK1); an inhibitory DAG kinase (DGK) such as DGKξ; or Janus kinase 2/signal transducer and activator of transcription 3 (Jak2/Stat3). In aspects, CEP(s) comprise TLR agonists (in aspects, such CEP(s) also comprise PD-1 PCI(s), e.g., anti-PD-L1 trap proteins or Abs). In aspects, CEPs comprise DAMPs, such as, e.g., calreticulin, HSPs (e.g., HSP70 or HSP90 PPTs), secreted amphoterin (HMGB 1), or ATP PPTs. In aspects, CEP(s) comprise hsc70, hsp110, grp170, or gp96 PPTs. In aspects, such PPTs are FPs. E.g., in aspects, a calreticulin (CRT) AARS acts as a TS in FPs in CEPs. In aspects, CEPs comprise innate IC IMs, e.g., anti-CSF1 PPTs, which can DOS promote development of M1 macrophages (e.g., anti-cancer macrophages). mAbs targeting CSF1R are known (FPA008, Five Prime Therapeutics; emactuzumab, Hoffmann-La Roche).

In aspects, CEP(s) comprise EP(s) that can be classified as innate trained immunity immunomodulator(s) (ITIIM(s)), either solely or in addition to other classes of EP(s) described elsewhere (e.g., PCMs, ITICSTAPs, etc.). ITIIMs are immunomodulator(s) that exhibit significant induction of IR(s) in or through ITICs. In aspects, ITIIMs are limited to those EPs that predominantly, generally, substantially or only exhibit significant IR(s) in or through ITICs. Several EPs described herein are ITIIs (e.g., EAT-2 PPTs and SAP PPTs). In aspects, ITIIM(s) are FP(s) including TS(s) that direct(s) FP(s) to ITICs. In aspects, TS(s) binds DCs, other ICs, and non-ICs (in aspects, such an FP is a N1-binding gDFP). In aspects, the TS is more specific for DCs than other cells, other ICs, or both (e.g., the FP comprises a DEC-205-binding domain). In aspects, PIM portions of such FP(s) comprise PPTs that block ITIC (e.g., DC) associated IL-10 expression/secretion, block DC-associated IL-IOR activity, block TGF-R binding/activity in ITICs/DCs, block IDO activity in DCs, block CCL22, block CCL17, block Bax apoptosis induction, block Bak apoptosis induction, comprise IL-12 PPTs, comprise MHCII PPTs, etc. In aspects, CEP(s) comprise chemokine(s) or related PPTs. Examples of such PPTs include macrophage inflammatory protein-ia (MIP-1a) and MIP-1b. In aspects, CEPs comprise IC-associated integrin PPTs or ligands, e.g., integrin-a4b1 PPTs or VCAM⁻¹ PPTs. Other ITIIMs include TLR modulators, e.g., TLR mimotopes.

### a. Exemplary Expression Products

To better illustrate aspects of the invention, specific EPs will be described in this section, including exemplary gDDs, ITIIs, CIs, Ags, etc.

### i. Pathogen Ags and Epitopes

In aspects, the EP capable of eliciting an immune response against a pathogenic disease-causing agent (DCA). An EP can comprise any suitable number and type of pathogen Ags. In aspects, some, most, generally all, or all of the Ags of an EP are directed to one type of pathogen. In aspects, an EP comprises at least two pathogen Ags, such as at least one MHC I and at least one MHC II Ags against the same type of pathogen. In aspects, OSMGAOA Ags of an EP are contained in FP(s), e.g., gDAgFP(s).

Exemplary Ags that can be encoded by AgESs in EPs include influenza Ags, such as nucleoprotein P, matrixprotein (M), and hemagglutinin (HA) Ags; Plasmodium Ags, such as thrombospondin-related anonymous protein, ring-infected erythrocyte surface antigen (RESA), merozoite surface protein 1 (MSP1), merozoite surface protein 2 (MSP2), merozoite surface protein 3 (MSP3), and glutamate-rich antigen (GLURP); and human papilloma virus (HPV) antigens, e.g., HPV-16 antigens, e.g., E5 protein, E6 protein, & E7 protein; and HIV antigens, such as gag, pol, nef, tet, and env.

In aspects, the DCA is one of the following pathogens: leishmania, Entamoeba histolytica (which causes amebiasis), trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Vaccines, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (e.g., Hantaviruses, Rift Valley Fever), Flaviruses (e.g., Dengue), Filoviruses (e.g., Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, California encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, and Trichomonas vaginalis. DCAs from which pathogen Ags can be obtained for inclusion in EPs include, without limitation, influenza virus, HIV, cytomegalovirus, dengue virus, yellow fever virus, tick-borne encephalitis virus, hepatitis virus, japanese encephalitis virus, human papillomavirus, coxsackievirus, herpes simplex virus, rubella virus, mumps virus, measles virus, rabies virus, polio virus, rotavirus, respiratory syncytial virus, Ebola virus, Chikungunya virus, Mycobacterium tuberculosis, Staphylococcus aureus, Staphylococcus epidermidis, E. coli, Clostridium difficile, Bordetella pertussis, Clostridium tetani, Haemophilus influenzae type b, Chlamydia pneumoniae, Chlamydia trachomatis, Porphyromonas gingivalis, Pseudomonas aeruginosa, Mycobacterium diphtheriae, Shigella, Neisseria meningitidis, Streptococcus pneumoniae and Plasmodium falciparum.

In aspects, CEPESCs comprise AgES(s) encoding Ag(s) of, and method comprise inducing IR(s) in swine against, a Porcine Epidemic Diarrhea Virus (PEDV) (e.g., by expressing Ag(s) contained in/encoded by Genbank NC_003436.1 or FVs thereof, such as GRSV(s) thereof). In aspects, CEPESC(s) comprise AgES(s) encoding Ag(s) against a porcine coronavirus, such as Swine Acute Diarrheal Syndrome coronavirus (SADS CoV) and methods comprise inducing IR(s) against porcine coronavirus(es), e.g., SADS CoV (e.g., by expressing Ag(s) contained in the AARS in/encoded by Genbank Accession MG605091 or FVs thereof, such as GRSV(s) thereof). In aspects, CEPESC(s) comprise AgES(s) encoding Ag(s) against Transmissible Gastroenteritis virus (TGEV) and methods comprise inducing IR(s) against TGEV (e.g., by expressing Ag(s) contained in the AARS in/encoded by Genbank Accession NC_038861.1 or FVs thereof, such as GRSV(s) thereof). In aspects, CEPESC(s) comprise AgES(s) encoding Ag(s) against Porcine deltavirus and methods comprise inducing IR(s) against Porcine Deltavirus (e.g., by expressing Ag(s) contained in the AARS in/encoded by Genbank Accession KJ481931.1 or FVs thereof, such as GRSV(s) thereof). In aspects, Ag(s) comprise, and methods include, inducing IR(s) against rotavirus, e.g., porcine rotavirus. Exemplary PMCs are provided in, e.g., Vlasova et al. Viruses 2017, 9, 48. In aspects, Ag(s) comprise Ag(s) of/related to porcine sapoviruses and methods comprise inducing IR(s) against the same. PMCs regarding Porcine Sapoviruses are found in Nagai et al. Diagnosis Virus Research 286 (2020) 198025.

Disorders that can be targeted by compositions and methods include Legionnaires' disease (Legionella), gastric ulcer (Helicobacter), cholera (Vibrio), E. coli infections, staphylococcal infections, salmonella infections or streptococcal infections, tetanus (Clostridium tetani), protozoan infectious diseases (malaria, sleeping sickness, leishmaniasis, toxoplasmosis, i.e. infections caused by plasmodium, trypanosomes, leishmania and toxoplasma), diphtheria, leprosy, typhoids, pertussis, rabies, tetanus, tuberculosis, typhoid, varicella, diarrheal infections such as Amoebiasis, Clostridium difficile-associated diarrhea (CDAD), Cryptosporidiosis, Giardiasis, Cyclosporiasis and Rotaviral gastroenteritis, encephalitis such as Japanese encephalitis, Wester equine encephalitis and Tick-borne encephalitis (TBE), fungal skin diseases such as candidiasis, onychomycosis, Tinea captis/scal ringworm, Tinea corporis/body ringworm, Tinea cruris/jock itch, sporotrichosis and Tinea pedis/Athlete's foot, Meningitis such as Haemophilus influenza type b (Hib), Meningitis, viral, meningococcal infections and pneumococcal infection, neglected tropical diseases such as Argentine haemorrhagic fever, Leishmaniasis, Nematode/roundworm infections, Ross river virus infection and West Nile virus (WNV) disease, Non-HIV STDs such as Trichomoniasis, Human papillomavirus (HPV) infections, sexually transmitted chlamydial diseases, Chancroid and Syphilis, Non-septic bacterial infections such as cellulitis, lyme disease, tetanus, tuberculosis, MRSA infection, pseudomonas, staphylococcal infections, Boutonneuse fever, Leptospirosis, Rheumatic fever, Botulism, Rickettsial disease and Mastoiditis, parasitic infections such as Cysticercosis, Echinococcosis, Trematode/Fluke infections, Trichinellosis, Babesiosis, Hypodermyiasis, Diphyllobothriasis and Trypanosomiasisplague, Anthrax Nipah virus disease, Hanta virus, Smallpox, Glanders (Burkholderia mallei), Melioidosis (Burkholderia pseudomallei), Psittacosis (Chlamydia psittaci), Q fever (Coxiella bumetii), Tularemia (Fancisella tularensis), rubella, mumps and polio. Coccidioidomycosis and swine (H1N1) influenza, sepsis such as bacteraemia, sepsis/septic shock, sepsis in premature infants, urinary tract infection such as vaginal infections (bacterial), vaginal infections (fungal) and gonococcal infection, foodborn illnesses such as brucellosis (Brucella species), Clostridium perfringens (Epsilon toxin), E. Coli O157:H7 (Escherichia coli), Salmonellosis (Salmonella species), Shingellosis (Shingella), Vibriosis and Listeriosis, rabies, pneumonia, pneumonic plague, acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, Cytomegalic inclusion disease, Kaposi sarcoma, multicentric Castleman disease, primary effusion lymphoma, AIDS, influenza, Reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, Poliomyelitis, Rabies, bronchiolitis, pneumonia, influenza-like syndrome, severe bronchiolitis with pneumonia, German measles, congenital rubella, Varicella, and herpes zoster. As such, EPs can comprise 1+ Ags against any of the DCAs that cause such disorders.

In aspects, a targeted DCA is a virus and EP(s) comprise(s) one or more viral Ags. Examples of viral Ags that can be included in EPs include herpes virus Ag proteins, such as gH, gL gM gB gC gK gE or gD proteins or Ag fragments or herpesvirus Immediate Early (IE) proteins such as HSV-1 or HSV-2 ICP27, ICP 47, IC P 4, or ICP36 from HSV1 or HSV2; cytomegalovirus proteins (such as CMV gB or derivatives thereof); Epstein Barr virus proteins (such as EBV gp350 or derivatives thereof); Varicella Zoster Virus proteins (such as gpI, II, III and IE63), or from a hepatitis virus such as hepatitis B virus (for example Hepatitis B Surface antigen (HBSAg) or Hepatitis core antigen or pol), hepatitis C virus antigen and hepatitis E virus antigen, or Ags of other pathogenic viruses, such as paramyxoviruses: Respiratory Syncytial virus (such as F and G proteins or derivatives thereof), or antigens from parainfluenza virus, measles virus, mumps virus, human papilloma viruses (for example HPVG, 11, 16, 18, eg L1, L2, E1, E2, E3, E4, E5, E6, or E7), flaviviruses (e.g. Yellow Fever Virus, Dengue Virus, Tick-borne encephalitis virus, Japanese Encephalitis Virus) or Influenza virus Ags (such as HA, NP, NA, or M proteins, or CT), or HIV Ags (such as tat, nef, gp120 or gp160, gp40, p24, gag, env, vif, vpr, vpu, or rev). Viral Ag(s) can comprise ≥1 Ags of a virus selected from Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. Viral Ag(s) can be from papilloma viruses e.g., human papillomoa virus (HPV), human immunodeficiency virus (HIV), polio virus, hepatitis B virus, hepatitis C virus, smallpox virus (Variola major & minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), HSV-1, herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, chikungunya virus, lassa virus, arenavirus, lymphocytic choriomeningitis virus (LCMV), or cancer causing virus. Additional viruses that can be targeted by compositions are described in US20200325182. DCAs can be associated with any suitable type of TR (e.g., dogs, pigs, or humans). In aspects, methods DOS reduce risk of species-to-species transmission of viruses by therapeutic or preventive administration of CEPESCs. For example, in one aspect the invention provides a method of reducing the likelihood or frequency of non-human species to human species transmission of virus comprising administration of CEPESCs to non-human TR(s), such as companion animals (e.g., dogs or cats), livestock, or both.

An EP can comprise a hepatitis antigen. A hepatitis antigen of an EP can be an antigen or immunogen from hepatitis A virus (HAV), hepatitis B virus (HBV), hepatitis C virus (HCV), hepatitis D virus (HDV), and/or hepatitis E virus (HEV). In some embodiments, the hepatitis antigen can be a heterologous nucleic acid molecule(s), such as a plasmid(s), which encodes one or more of the antigens from HAV, HBV, HCV, HDV, and HEV. An HAV Ag can comprise a capsid protein, a HAV non-structural protein, a fragment thereof, a variant thereof, or a combination thereof. An HCV Ag can comprise a nucleocapsid protein (i.e., core protein), an HCV envelope protein (e.g., E1 and E2), an HCV non-structural protein (e.g., NS1, NS2, NS3, NS4a, NS4b, NS5a, and NS5b), a fragment thereof, a variant thereof, or a combination thereof. An HVD antigen can be a HDV delta antigen, fragment thereof, or variant thereof. A HEV Ag can be a HEV capsid protein, fragment thereof, or variant thereof. An HBV Ag can be an HBV core protein, an HBV surface protein, an HBV DNA polymerase, an HBV protein encoded by gene X, fragment thereof, variant thereof, or CT. An HBV Ag can be from any HBV genotype (e.g., genotype(s) A-H).

An HPV antigen can be any suitable HPV Ag. HPV Ags can be from, e.g., HPV types 16, 18, 31, 33, 35, 45, 52, and 58 which cause cervical cancer, rectal cancer, and/or other cancers. The HPV antigen can be from HPV types 6 and 11, which cause genital warts, and are known to be causes of head and neck cancer. HPV antigens can be the HPV E6 or E7 domains from each HPV type. For example, for HPV type 16 (HPV16), the HPV16 antigen can include the HPV16 E6 antigen, the HPV16 E7 antigen, fragments, variants, or combinations thereof. Similarly, the HPV antigen can be HPV 6 E6 and/or E7, HPV 11 E6 and/or E7, HPV 18 E6 and/or E7, HPV 31 E6 and/or E7, HPV 33 E6 and/or E7, HPV 52 E6 and/or E7, or HPV 58 E6 and/or E7, fragments, variants, or combinations thereof. In one embodiment an HPV antigen such as an E6 or E7 antigen disclosed herein is selected from an HPV 6 strain, and HPV 11 strain, HPV 16 strain, an HPV-18 strain, an HPV-31 strain, an HPV-35 strain, an HPV-39 strain, an HPV-45 strain, an HPV-51 strain an HPV-52 strain, an HPV-58 strain or an HPV-59 strain. In another embodiment, the HPV antigen is selected from a high-risk HPV strain. In another embodiment, the HPV strain is a mucosal HPV type. In another embodiment, HPV antigens can be selected from all HPV strains, including non-oncogenic HPVs such as type 6, 11, etc. that cause warts and dysplasias. In another embodiment, the HPV antigen is an HPV-31, HPV-35, HPV-45, HPV-51, HPV-52, or HPV-58 Ag. In aspects, an HPV E6 antigen is utilized instead of or in addition to an E7 antigen.

An RSV antigen can be a human RSV fusion protein (also referred to herein as “RSV F”, “RSV F protein” and “F protein”), or fragment or variant thereof. Examples of RSV Ag(s) are described in US20200325182.

Viruses include DNA or RNA animal virus. As used herein, RNA viruses include, but are not limited to, virus families such as picornaviridae (e.g., polioviruses), reoviridae (e.g., rotaviruses), togaviridae (e.g., encephalitis viruses, yellow fever virus, rubella virus), orthomyxoviridae (e.g., influenza viruses), paramyxoviridae (e.g., respiratory syncytial virus (RSV), measles virus (MV), mumps virus (MuV), parainfluenza virus (PIV)), rhabdoviridae (e.g., rabies virus (RV)), coronaviridae, bunyaviridae, flaviviridae (e.g., hepatitis C virus (HCV)), filoviridae, arenaviridae, bunyaviridae, and retroviridae (e.g., human T-cell lymphotropic viruses (HTLV), human immunodeficiency viruses (HIV)). DNA viruses include, but are not limited to, virus families such as papovaviridae (e.g., papilloma viruses), adenoviridae (e.g., adenovirus), herpesviridae (e.g., herpes simplex viruses, e.g., HSV-1, HSV-2; varicella zoster virus (VZV); Epstein-Barr virus (EBV); cytomegalovirus (CMV); human herpesviruses, e.g., HHV-6 and HHV-7; Kaposi's sarcoma-associated herpesvirus (KSHV) and the like), and poxviridae (e.g., variola viruses). See, e.g., Knipe et al., Field's Virology, 4th ed., Lippincott Williams & Wilkins, 2001.

In aspects, a viral DCA is an enveloped virus. Examples of enveloped viruses from which one or more Ags for inclusion in EPs can be obtained include Human Immunodeficiency Virus 1 (HIV-1), Human Immunodeficiency Virus 2 (HIV-2), Herpes 1 virus (HSV-1), Herpes 2 virus (HSV-2), Influenza Virus, Respiratory Syncytial Virus (RSV), Cytomegalovirus (CMV), Zika Virus (ZKV), Dengue Virus, West Nile Virus, Lassa Virus, Ebola Virus, Marburg virus, Porcine Reproductive And Respiratory Syndrome Virus, Poxvirus, Bovine Viral Diarrhea Norovirus, SARS Coronavirus, Chikunguya Virus, Hepatitis C Virus, Hepatitis B Virus, African swine fever virus, Eastern Equine Encephalitis Virus, Cowpox virus, Western Equine Encephalitis Virus, Nipah Virus, Human parainfluenza viruses, Japanese Encephalitis Virus, Tick Borne Encephalitis Virus, Newcastle Virus, Border Disease Virus (BDV) of sheep, Classical Swine Fever Virus (CSFV), etc. See further US20200325182.

In aspects, a viral Ag is a fully or partially glycosylated surface glycoprotein, or fragment thereof, or a sequence of a typically glycosylated viral glycoprotein that is at least partially or fully deglycosylated. Examples of such Ags include influenza virus neuraminidase, influenza virus hemagglutinin, influenza virus M2 protein, human respiratory syncytial virus (RSV)-viral proteins, RSV F glycoprotein, RSV G glycoprotein, herpes simplex virus (HSV) viral proteins, herpes simplex virus glycoproteins gB, gC, gD, and gE. Additional examples are provided in US20200325182. In aspects, an EP comprises a Lymphocytic Choriomeningitis Virus (LCMV) Antigen. An LCMV Ag can be an antigen from, e.g., LCMV Armstrong.

To further illustrate aspects wherein EPs comprise one or more viral Ags, specific exemplary aspects are provided in the following sections.

### 1) PCV

In aspects, constructs comprise AgES(s) encoding Ag(s) of/against porcine circovirus (PCV). EP(s) can comprise any suitable number of PCV Ag(s) against any suitable type of PCV (e.g., PCV-1, PCV-2, or PCV-3, any one or more subtypes thereof, or combinations of any thereof).

In aspects, OSMGAOA PCV Ag(s) of CEPs are associated with PTPS(s), such as one or more polyUb sequences. In aspects, NS(s) comprising PCV AgESs are mostly, generally, or only contained in NAV(s). In aspects, NAV(s) are plasmids. In aspects, NSs comprise EEI(s) associated with the PCV AgES, gDP(s), or both. In aspects, OSMGAOA PCV Ag(s) are delivered in gDAgFP(s). In aspects, the gD portion of OSMGAOA gDAgFP(s) of such CEPESCs exhibit preferred/selective affinity for gD receptor(s) (gDR(s)) over HVEM (e.g., nectin-1, nectin-2, or homologs). In aspects, OSMGAOA gDP(s) in such CEPs exhibit significantly reduced HVEM binding regarding to HSV-1 gD, do not exhibit significant binding of HVEM, or both. In aspects, OSMGAOA of gDAgFP(s) of such CEPs lack a functioning gD TMD. In aspects, gDAgFP(s) or other EP(s) comprise EP(s) comprising 2+, 3+, or ≥4 PCV Ag(s), wherein the PE optionally includes linker(s) (e.g., MSL(s), FL(s), or MSFL(s)), cleavage sites (e.g., SCS(s), such as 2A sites), or CT. In aspects, EP(s) include TS(s), e.g., ITS(s), such as an ERTPS, an exosome TS, PTPS(s), or CT. In aspects, OSMGAOA PCV Ag AgES(s) are contained in one or more viral vectors. In aspects, the viral vector is a vector comprising primarily or generally comprising non-pathogenic PCV1 genes but containing anti-PCV2 Ags, anti-PCV3 Ags, or CT. In aspects, PCV Ag(s) are chimeras/hybrids comprising Ags of 2+ PCVs, composed of AARSs of 2 PCVs, or CT (examples of hybrid PCV Ags are exemplified in, e.g., Opriessnig T et al, 2014, CEH). In aspects, PCV Ag(s) comprise hybrid Ag(s) (e.g., PCV-2 Ag(s) and PCV-3 Ag(s)). Other hybrid Ags can be generated by, e.g., combining effective portions of bat circovirus genomes known to be related to PCV with PCV2 or PCV3 AgESs.

In aspects, OSMGAOA PCV Ag(s) of a CEP are AgV(s). In aspects, AgV(s) comprise editope(s). In aspects, PCV AgV(s) of CEPs ACA based on comprising GSRV(s). In aspects, GSRV(s) in PCV Ag(s), and optionally also in other EP(s), result in the CEP being AW a DOS reduction in humoral antigenicity; enhanced efficacy in promoting, inducing, or enhancing one or more aspects of IR(s); and reduced likelihood of CSAE(s), e.g., cytokine storm syndrome, Vaccine-Associated Enhanced Respiratory Disease (“VAERD”), or combination of any or all thereof (CT/combination).

In aspects, NSs of such CEPESCs are contained in plasmids that are at least primarily or generally associated with TFA(s), such as a CaPNP(s). In methods, the delivery of such CEPESCs to TR(s) comprises, PC, GCO, or CO mucosal administration of CEPESC(s) to TR(s) (e.g., pigs, humans, or both).

In aspects, CEPs comprise PCV1 Ag(s). In aspects, CEPs comprise PCV2 Ag(s) & PCV3 Ag(s); PCV2 Ag(s) & PCV1 Ag(s); PCV1 Ag(s) & PCV3 Ag(s); or a combination of PCV1, PCV2, and PCV3 Ags. In aspects, such Ag(s) comprise AgV(s), e.g., GSRAgV(s), e.g., a PCV1 ORF1 GSRAgV, a PCV1 ORF4 GSRAgV, a PCV1 ORF2 GSRAgV, or CT.

In aspects, PCV Ag CEPs comprise non-gD PCM(s), such as PCI(s). In aspects, a PCV Ag EP will comprise a PD-L1 antagonist CI AARS or PPT. In aspects, a PCV Ag CEP comprises ICSTAPs, e.g., EAT-2 or SAP PPTs.

In aspects, administration of a PCV AgES CEPESC to TR(s) is repeated two or more times. In aspects, administration of a PCV AgES CEPESC is combined with the administration of an anti-PCV vaccine in a prophylactic method. In aspects, the administration of a PCV AgES CESESC is combined with one or more anti-viral therapies or therapies for the symptoms of PCV.

Administration of the PCV AgES CEPESCs induces DOS IR(s) against PCV(s). In aspects, such IR(s) comprise a significant anti-PCV T cell IR, a significant anti-PCV Ag B cell IR, or both. In aspects, a PCV Ag CEP will comprise MHCIE(s), MHCIIE(s), BCE(s), or a combination of 2 or 3 thereof.

In aspects, delivery of PCV AgES CEPESC(s) induces IR(s) against at least two subtypes of PCV (e.g., a combination of 2 or 3 of PCV2a, PCV2b, and PCV2d), two or more types of PCV (e.g., PCV2 and PCV3), or both. In aspects, the PCV Ag EPESC will elicit effective anti-PCV B and T cell immunity and durable memory responses that will recognize and destroy field-relevant PCV strains upon primary infection; clear PCV infected cells from chronically infected pigs harboring endogenous PCV (reservoir) when the virus emerges from latency; or both, in DOS levels. In aspects, the administration of an effective amount of a PCV CEPESC one or more times to TR(s) (e.g., a pig or population of pigs) results in DOS improvement in aspect(s) of the prevention, treatment, or both of one or more PCV-associated conditions, such as postweaning multisystemic wasting syndrome (PMWS), PCV2-systemic disease (PCV2-SD), or other porcine circovirus diseases (PCVDs) or an improvement in one or more clinical symptoms or other aspects thereof. For example, administration of a PCV Ag EPESC can result in a detectable or significant improvement in lymphoid depletion, lymphoid inflammation, positive IHC for PCV2 antigen of lymphoid tissue, viremia, nasal shedding, pyrexia, reduced average daily weight gain, lung inflammation, positive IHC for PCV2 antigen of lung tissue, or mortality. In aspects, an anti-PCV AgES CEPESC will exhibit DOS PCV Ag-specific IR(s) at about 7 days post treatment (pt) or post challenge inoculation (pi); also at ˜14 days pt or pi; also at ˜28 days pt or pi; also at ˜45 days pt or pi; also at ˜60 days pt or pi; also at ˜90 days pt or pi; also ˜6 months pt or pi; also ˜12 months pt or pi; also ˜18 months pt or pi; also ˜2 years pt or pi; or also ˜3 years pt or pi. In aspects, an anti-PCV EP protected or treated pig will exhibit a reduced PCV qPCR measurement after any such period after one, two, or more PCV challenges. In aspects, an anti-PCV EP will result in a detectably or significantly reduced amount of total PCV-associated lymphoid lesions in infected or challenged pigs. In aspects, an anti-PCV EP will result in a detectably or significantly reduced number of lesions that are considered severe lesions according to standard classification practice. In aspects, an anti-PCV Ag EP will result in one or more improvements in PCV health-related indicators in a CDCD pig model as compared to similar untreated pigs under similar conditions, Circovac®-treated pigs under similar conditions or both. In aspects, an anti-PCV EP treated or protected pig will exhibit a reduction in PCV as determined by sera testing, fecal/rectal swabbing, or both, in any such period, with analysis performed by viral counting methods, ELISA, and the like. Measurement of indicators of PCV infection are described in, e.g., Palya V. Virol J. 2018; 15(1):185. In aspects, delivery of an effective amount (EA) of anti-PCV EP as a protective method, therapeutic method or both, one or more times, results in a DOS reduction in PCV-associated disease (PCVAD) in a population of swine or a detectable or significant reduction of one or more aspects/symptoms of PCVAD such as a reduction of wasting in PCV-2 associated pigs. In aspects, an anti-PCV EP also results in cross-protective immune responses for two, three, or more types of PCV.

In aspects, treatment or prophylaxis of PCV infection by administering an effective amount of PCV AgES CEPESC(s) results in DOS improvement in comparison to animals of a non-treated control group of the same species/class in a vaccine efficacy parameter selected from the group consisting of a reduction in loss of weight or reduced weight gain, a shorter duration of viremia, an earlier end to viremia, a lower virus load, in delay of onset of viremia, in a reduced viral persistence, in a reduction of the overall viral load and/or a reduction of viral excretion, or combinations of any or all thereof. In aspects, administration of the PCV Ag EPESC also results in detectably or significantly reducing lymphadenopalhy, lymphoid depletion and/or multinucleated/giant histiocytes in animals infected with PCV or subsequently challenged with PCV. In aspects, administration of the PCV Ag EPESC results in detectably or significantly reducing (1) interstitial pneumonia with interlobular edema, (2) cutaneous pallor or icterus, (3) mottled atrophic livers, (4) gastric ulcers, (5) nephritis (6) reproductive disorders, e.g. abortion, stillbirths, mummies, etc., (7) Pia like lesions, normally known to be associated with Lawsonia intracellularis infections (Ileitis), (8) lymphadenopathy, (9) lymphoid depletion, (10) multinucleated/giant histiocytes, (11) Porcine Dermatitis and Nephropathy Syndrome (PDNS), (12) PCVAD associated mortality, (13) PCVAD associated weight loss, (14) reduced growth variability (15), reduced frequency of ‘runts,’ (16) reduced co-infections with Porcine Reproductive and Respiratory Disease Complex (PRRSV), or (17) a combination of any or all thereof in a PCV infected or subsequently challenged pig (or both). In aspects, administration of a PCV AgES CEPESC results in DOS improvement in one or more economic/growth parameters in PCV infected or PCV challenged pigs such as time to slaughter, carcass weight, improvement in average weight gain, lean meat ratio, or a combination of any or all thereof. In aspects, delivery of the PCV Ag EPESC reduces the overall circovirus load including a later onset, a shorter duration, an earlier end of viremia, and a reduced viral load and its immunosuppressive impact in young animals, in particular in those having anti-PCV2 antibodies at the day of vaccination, thereby resulting in a higher level of general disease resistance and a reduced incidence of PCV2 associated diseases (e.g., PRRSV, swine influenza virus (SIV), and Mycoplasma hyopneumoniae, or a combination of any or all thereof) and symptoms. In aspects, administration of a PCV Ag EPESC also results in a detectable or significant reduction in wasting, severe wasting, enlarged lymph nodes, respiratory illness, jaundice, or combination thereof.

In aspects, PCV AgES CEPESC(s) are administered in an effective amount to a piglet (an un-weaned pig or a pig that is about 2 months in age or less). In aspects, an effective amount of a PCV Ag EPESC is administered to a population of pigs that still exhibits a significant amount of material anti-PCV2 antibodies. In aspects, a PCV Ag EPESC is administered to a population of swine that has an average age of ≤˜12 weeks in age, ≤˜10 weeks in age, ≤˜8 weeks in age, ≤˜7 weeks in age, less than about 6 weeks in age, ≤˜5 weeks in age, or ≤˜4 weeks in age (e.g., 1-10 weeks, 2-10 weeks, 3-9 weeks, 2-8 weeks, 1-8 weeks, 2-6 weeks, 1-7 weeks, 1-6 weeks, 1-4 weeks, or 2-4 weeks in age). In aspects, the PCV Ag EPESC is delivered to pigs of between about 1-30 days of age, such as about 2-30, about 3-30, about 1-21, about 3-21, about 2-24, or about 2-22 days of age. In aspects, all pigs of a population treated with a PCV Ag EPESC are within such an indicated range or below such an indicated maximum age (e.g., 6 weeks, 4 weeks, or 3 weeks of age).

In aspects, an at least as effective immune response, detectably or significant better immune response, or detectably or significantly improved clinical outcome in any of the above-described aspects is achieved from the delivery of an effective amount of a PCV Ag EPESC to pigs with a lower incidence of adverse events (e.g., a lower incidence of cytokine storm events), a lower number of required or required average administrations of medicament, or both, than with a current on-market anti-PCV vaccine, anti-PCV immunogen therapy, or any such therapy or vaccine currently in the art.

In aspects, compositions comprising EPs expressing one or more PCV CRAs are provided. Putative PCV Ag constructs can be developed from such materials identified from methods such as ELI, etc. (and which can be optionally combined with known PCV Ags), leading to preparation of test CEPESCs comprising corresponding AgESs, typically comprising ES(s) for gDP(s) (e.g., gDAgFP(s) comprising putative PCV Ag(s)). Such CEPESCs can then be tested in clinical studies to identify CRAs. CRAs meeting pre-determined standards can be analyzed and sequenced to identify PCV epitopes and develop AgV(s), such as editope(s)/GSRAgV(s).

In aspects, the PCV Ag EPESC comprises, PC, or only comprises NAM(s) that are NAV(s). In aspects, NAV(s) are associated with TFA(s) that DOS enhance IR(s) in TR(s). In aspects, such TFA(s) comprise CaPNP(s).

The PCV treatment and prophylaxis/vaccination methods also represent 1 facet of the aspects directed to treatment of leaky vaccine-associated disorders. E.g., although PCV2 vaccines can reduce clinical signs of PCVAD, such on-market vaccines are “leaky” and do not provide sterilizing immunity. As such, pigs vaccinated with currently available vaccines can still become infected with PCV2 and spread the virus. Moreover, such leaky vaccines at least typically fail to confer protection from repeated PCV exposure, coinfection, and mutations that foster the spread of virus within herds and increase the PCV reservoir in a population. Use of PCV Ag EPESCs can surprisingly overcome SMGAOA aspects of such leaky vaccine associated challenges, such as detectably, generally completely, substantially completely, or completely eliminating transmission of PCV from chronically infected animals; prevent infection in a significant fraction of a herd, independent of the number of relevant PCV exposures over the lifetimes of individuals; or both.

In aspects, CEPs comprise PCV2 Ag(s). In aspects, CEPs comprise PCV2a Ag(s), PCV2b Ag(s), PCV2c Ag(s), PCV2d Ag(s), PCV2d-mPVCC2b Ag(s), or PCV2e Ag(s). Classification of PCV is still developing and may change in the future (SFE Franzo G, Virol J. 2015; 12:131), but the efficacy of CEPs against PCVs is expected to apply regardless of such changes in classification of PCV species/strains. The biology of many PCV strains is known and adaptable to aspects (SFE Chen F et al. J Virol. 2012; 86(22):12457-12458, with respect to a strain of PCV2d; Cheung A K. Virology. 2003; 305(1):168-180, describing, i.a., PCV2 strain 688, which has a 236 AAR ORF2 (see also UniProt Access No. 056129); and Hamel, A. L. et al. J. Virol. 72 (6), 5262-5267 (1998) (providing the complete genome of a PCV2 virus) (see also Trible B R, Virus Res. 2012; 164(1-2):68-77 and Karuppannan A K. Viruses. 2017; 9(5):99. Published 2017 May 6). Numerous additional PCV strains have been sequenced and characterized in GenBank (SFE Access Nos. JX535296, JX519293, MG833033, and JQ653449 (with respect to PCV2d isolates, for example). In aspects, CEPs comprise Ag(s) of/against a PCV “mutant” strain, such as the US isolated mPCV2 (Opriessnig T et al. J Gen Virol. 2014; 95(Pt 11):2495-2503) or the enhanced virulence PCV2b-234-K variant (K addition in ORF2) strain (SFE Guo L et al. PLoS One. 2012; 7(7): e41463).

In aspects, CEPs comprise one of a PCV-2 ORF1, ORF2, ORF3, or ORF4 or a fragment of any thereof (e.g., a fragment comprising S, M, or GA thereof). In aspects, CEPs comprise PCV2d Ag(s) or PCV2b Ag(s). In aspects, CEPs comprise PCV Ags against 2+ PCV2 subtypes. In aspects, CEPs comprise a PCV2 ORF3 EP (e.g., EL ORF3, GA of ORF3, most of ORF3, or some of ORF3). In aspects, CEPs comprise a PCV Rep, Rep′, or Cap protein, etc. In aspects, PCV Ag CEPs comprise PCV2 Ag(s) from at least two PCV2 ORFs, such as any 3 or 4 PCV2 ORFs. In aspects, PCV2 Ag CEPs include PCV2 ORF2 Ag(s) and non-ORF2 PCV Ag(s), e.g., ORF1 Ag(s), ORF3 Ag(s), ORF4 Ag(s), or CT, such as an ORF3 Ag and ORF1 Ag or ORF4 Ag. In aspects, PCV2 Ag CEPs comprise ORF2 Ag(s) & ORF3 Ag(s). In aspects, CEPs include PCV2a Ag(s) & PCV2b Ag(s). In aspects, a PCV2 Ag CEP include PCV2 BCE(s), e.g., BCE(s) AW anti-PCV2 neutralizing Abs. In aspects, any such PCV-2 Abs preferably bind endogenous PCV over EPs or do not significantly bind EPs or do not DOS impair MGAOA of the IR(s) induced by EP(s). BCEs of PCV are known; examples of such BCEs include PCV2 capsid AAs 65-87, 113-139, 169-183, and 193-207.

PCV2 Ags and related compositions and methods that can be combined with aspects of this disclosure are known in the art. For example, WO03/049703 describes production of a live chimeric vaccine is described, comprising a PCV-I backbone in which an immunogenic gene of a pathogenic PCV2 strains replaces a gene of the PCV-I backbone. WO99/18214 describes several PCV2 strains. An ORF-2 based subunit vaccine has been reported in WO2006/072065 and in WO2007/028823. A PCV2b ORF2 protein is disclosed in WO2019025519. Additional PCV2 antigens are provided in WO2006/072065 (e.g., SEQ ID NOs: 11, 10, 9, or 5 therein). Nucleotide sequences for such Ags also are KNOWN (e.g., SEQ ID NOs: 3 and 4 therein). Additional PCV2 Ags, sequences, and methods are described in, e.g., US20180236057; US20150056248; US20170232094; US20160206727; WO2008076915; U.S. Ser. No. 10/131,696 and U.S. Pat. No. 9,932,372. PCV2 T cell epitopes are described in, e.g., Stevenson L S et al. Viral Immunol. 2007; 20(3):389-398 and Wyatt, C., 2009, Mapping T cell epitopes in PCV2 capsid protein, National Pork Board Report, available at pork.org/research/mapping-t-cell-epitopes-in-pcv2-capsid-protein/. PCV Ags, epitopes, and related compositions are disclosed in, e.g., WO2017/187277; UniProt Access Number F5A4Z4; CN103536912; Mahé D et al. J Gen Virol. 2000; 81(Pt 7):1815-1824; and Bandrick M et al. Vet Immunol Immunopathol. 2020; 223:110034. PCV Ag EPs can comprise any PCV Ag(s) or epitope(s) described in these references or that are otherwise KNOWN. Methods for treating PCV infections and preventing PCV infections can employ any suitable methods, compositions, or standards therein.

In aspects, CEPs comprise PCV2 AgV(s), e.g., PCV2 editope(s). An example of such an AgV is a variant comprising a modification at position 143, 145, or both, typically position 143, resulting in deletion of a potential NLGS. In aspects, PCV2 AgV(s) exhibit enhanced antigenicity as compared to WTC(s). An example of such an editope is the chimeric PCV2 ORF2 protein in the chimeric virus PCV2-3cl, generated through application of gene shuffling methods (see WO2017187277), or a related antigenic virus developed by application of such gene shuffling techniques (e.g., a PCV2 capsid polypeptide selected from the PCV capsid polypeptides designated as 3cl.14 (SEQ ID NO: 8 of WO2017187277), 3dl. 13 (SEQ ID NO: 4 of WO20 17187277), 3cl.4_2 (SEQ ID NO: 2 of WO2017187277), and 3 cl. 12_2 (SEQ ID NO: 6 WO2017187277).

In aspects, CEPs comprising PCV2 Ag(s) that, i.a., lack of any PCV2 decoy epitopes or are modified to remove the same (through substitution or deletion). PCV2 decoy epitopes are known and described elsewhere. In aspects, CEPs comprise Ags from/related to PCV2 ORF9. In aspects, CEPs comprise an AARs RVRHRSIOI to SEQ ID NO:654.

In aspects, an EP include(s) ≥1 Ags from or that are at least related to a PCV2 ORF1 sequence. In aspects, an EP includes a PCV2 Ag including a sequence RVRHRSIOI to SEQ ID NO:654. In aspects, CEPs comprise a GSRAgV(s) of an ORF1 sequence, e.g., 1 or ≥1 of SEQ ID NOs:655-657. In aspects, CEPs comprise Ag(s) of related to a PCV2 ORF3 sequence. In aspects, CEPs include Ag(s) RVRHRSIOI to SEQ ID NO:658.

In aspects, CEPs comprise Ag(s) from or related to a PCV2 ORF4 PPT. In aspects, CEPs comprise PCV2 Ag(s) RVRHRSIOI to SEQ ID NO:659. In aspects, CEPs comprise GSRVs of ORF4 PPTs (e.g., SEQ ID NO:660) or FFs/FVs. In aspects, CEPs comprise Ag(s) from or related to a PCV2 ORF8 PPT. In aspects, CEPs comprise PCV2 Ag(s) are RVRHRSIOI to SEQ ID NO:661. In aspects, CEPs comprise Ag(s) from or related to a PCV2 ORF11 PPT. In aspects, CEPs comprise PCV2 Ag(s) RVRHRSIOI to SEQ ID NO:662. In aspects, CEPs comprise Ag(s) from or related to a PCV2 ORF5 PPT. In aspects, CEPs comprise a PCV2 Ag RVRHRSIOI to SEQ ID NO:663. In aspects, CEPs comprise Ag(s) from or related to PCV2 ORF10 PPTs. In aspects, comprise PCV2 Ag(s) RVRHRSIOI to SEQ ID NO:664.

In aspects, CEPs comprise Ag(s) from or related to PCV2 ORF7 PPTs. In aspects, CEPs comprise PCV2 Ag(s) RVRHRSIOI to SEQ ID NO:665.

In aspects, CEPs comprise Ag(s) from or related to a PCV2 ORF2 PPT. In aspects, CEPs comprise PCV2 Ag(s) RVRHRSIOI to SEQ ID NO:24. In aspects, CEPs comprise PCV2 ORF2-related GSRV Ag(s) (e.g., SEQ ID NO:25 or SEQ ID NO:666). In aspects, CEPs include PCV2 ORF2 FVs per the formula MTYPRRRX₈RRRRHRPRSHLGX₂₁ILRRRPWLVHPRHRYRWRRKNGIFNX₄₇RLSRX₅₂X₅₃X ₅₄YTX₅₇X₅₈X₅₉X₆₀X₆₁VX₆₃TPSWAVDMMRFX₇₅X₇₆X₇₇X₇₈FVPPGGGX₈₆NX₈₈X₈₉X₉₀IPFEYY RIRKVKVEFX₁₀₆X₁₀₇X₁₀₈SPITQGDRGVGSTAVILX₁₂₆DNFVTKATALTYDPYVX₁₄₃YSSRH TIPQPFSYHSRYFTPKPVLDSTIDYFQPNNKRNLWLRLQTX₁₉₀X₁₉₁NVDHVGLGX₂₀₀ AFX_{20 3}NSX₂₀₆X₂₀₇X₂₀₈QX₂₁₀YNX₂₁₃RX₂₁₅TMYVQFREFNLKDPPLX₂₃₂X₂₃₃X₂₃₄X₂₃₅ (SEQ ID NO:668), wherein X8 is Y or F, typically Y; X21 is Q, L, or H, typically Q; X47 is T, A, or S, typically T/A; X52 is T or S; X53 is F or I, typically F; X54 is G or V; X57 is V or I, typically V; X58 is K or N; X59 is A or R; X60 is T or S; X61 is T or Q; X63 is R, S, or T; X75 is D, N, or K, typically D/N; X76 is I or F, typically I; X77 is D or N; X78 is D or Q; X86 is T or S; X88 is K or P; X89 is I, R, or L; X90 is S or T; X106 is W of R; X107 is P or A; X108 is C or R; X126 is T or S, usually T; X143 is D or N, usually D; X190 is S, A, or T, typically S or T; X191 is R, G, or K, typically R or G; X200 is T or H; X203 is E or Q; X206 is I, T, or K, typically T; X207 is Y or N; X208 is D or A; X210 is D, E, or A, typically D or A; X213 is I or V; X215 is I or V, typically V; X232 is K or N, typically K; X234 is P, K, or is absent, typically P or K; & X235 is K or is absent.

In aspects, PCV2 AgES CEPs encode PCV2 Ag mimotope(s). Examples of PCV2 Ag mimotopes and related PMCs are in, e.g., Hung L C et al. BMC Immunol. 2017; 18(1):25 and WO2006/072065.

In aspects, PCV2 Ag EPs can also comprise one or more hybrid Ags comprising one or more non-PCV2 Ags. Such methods can be relevant given the prevalence of PCV2 co-infections, such as SIV & PRRSV. As such, in aspects, PCV Ag EPs additional comprise Ag(s) of/against any such potential PCV co-infection agents. Examples of hybrid PCV2 Ags, e.g., are described in, e.g., Piñeyro P E et al. Virus Res. 2015; 210:154-164 and Li X et al. Appl Microbiol Biotechnol. 2018; 102(24):10541-10550.

In aspects, a PCV2 Ag EP includes polyepitope FP(s). In aspects, one, some, generally all, or all of such polypeptide sequences will be contained in gDAgFPs. In aspects, at least one polyepitope sequence will comprise one or more linkers (e.g., mid-sized or flexible linkers), one or more cleavage sites, one or more PTPSs (e.g., one or more polyUb sequences) or other ITSs, or a combination of any or all thereof. In aspects, at least one Ag of a polyepitope sequence will comprise an Ag variant, such as a deglycosylation variant. In aspects, PE(s) will comprise Ag(s) from at least two, ≥3, ≥4, ≥5, ≥6, or at least seven PCV2 ORFs (e.g., 2-8 PCV2 ORFs, 2-7 PCV2 ORFs, 2⁻⁵ PCV2 ORFs, or 2-3 PCV2 ORFs). In aspects, a polyepitope sequence also will comprise at least two, at least three, at least four, at least five, at least six, at least seven or more discrete Ag sequences (e.g., 2-12, 2-10, 2-8, 2-7, 3-15, 3-12, 3-9, or 3-7 discrete Ag sequences), most, generally all, or all of which are PCV2 Ag sequences (including variants). E.g., a construct according to the formula gD1-optional linker-ORF9Ag-linker-ORF4Ag-linker-ORF8Ag-linker-ORF11Ag-linker-ORF10Ag-linker-ORF6Ag-linker-ORF7Ag-optional linker-gD2.

Several vaccines against PCV2 are commercially available. Porcilis® PCV (available from MSD Animal Health, Boxmeer, The Netherlands) is a vaccine for protection of pigs against porcine circo virus type 2, for use in pigs from three weeks and older. When given as a two-shot (two dose) vaccine, the duration of immunity (DOI) is 22 weeks. CircoFlex® (available from Boehringer Ingelheim, Ingelheim) is a vaccine for protection of pigs against porcine circo virus type 2, for use in pigs from two weeks and older. It is registered as a one-shot (one dose) vaccine only. Circovac® (available from Merial, Lyon, France) is a vaccine for protection of pigs against porcine circo virus type 2, for use in pigs three weeks and older. Suvaxyn® PCV (available from Zoetis, Capelle a/d IJssel, The Netherlands) is a vaccine for protection of pigs against PCV2, for use in pigs 3 weeks and older. Other PCV2 vaccines are described in WO2007/028823, WO 2007/094893, and WO2008/076915. All on-market recombinant vaccines target the immunogenic capsid protein of the virus encoded by ORF2, while the conventional, inactivated vaccines are whole virus preparations (Beach and Meng, 2012). Such vaccines can be combined with a PCV AgES CEPESC or can be administered in AW PCV AgES CEPESC(s).

In aspects, an effective amount of a PCV2 Ag EPESC will exhibit a detectably or significantly improved immune response in one or more aspects as compared to a recommended dose (e.g., 0.5 ml in piglets or 2 ml in mature pigs) of Circovac®, an FDA approved PCV treatment or over any of the other above-described approved treatment/vaccines administered according to their recommended application regimens. In aspects, EPs exhibit a better immune response in one or more respects against more types of PCV2 viruses. In aspects, EPs also exhibit an immune response that is detectably better or significantly better than Circovac® against PCV2A viruses. In aspects, the improved immune response comprises a significantly better memory immune response than Circovac® or any other 1+, 2+, or all of the other above-described commercial on-market vaccines. In aspects, the improved immune response comprises a significantly better T cell response, a significantly better innate trained immune response, or both, as compared to any one, some, or all of such on-market vaccines. In aspects, an EP will result in significantly reduced shedding, significantly reduced PCV-associated viremia, or both, as compared to any one, some, or all of such vaccines, and in aspects administration of an PCV2 Ag EPESC will result in significantly improved shedding reduction, significantly reduced PCV-associated viremia, or both, as compared to Circovac® or any other OSMOA of the on-market PCV2 vaccines. In aspects, an EP will exhibit any of the above-described improved responses as compared to Circovac® or any other one, some, or all of the on-market PCV2 vaccines/treatments in PCV2d pigs. Clinical experience with Circovac® in PCV2a and PCV2d pigs adaptable to aspects is known (See, e.g., Opriessnig T et al. Vaccine. 2017; 35(2):248-254). In aspects, a PCV Ag EP will comprise a PPT comprising an AARS that is at least about 70%, 80%, 85%, 90%, 95%, 97%, or 99% identical to an at least 8 AA AARS, an at least 12 AA AARS, an at least 15 AA AARS, ≥20 AA AARS, ≥50 AA AARS, or ≥100 AA AARS in, e.g., SEQ ID NO:24 and that induces significant IR(s) against PCV(s) when expressed in TR(s). In aspects, an EP comprises SEQ ID NO:25. In aspects, an EP comprises SEQ ID NO:24.

In aspects, an EP will also comprise one or more PCV3 Ags. PCV3 has recently been identified as a virus that is distinct from and distantly related to other PCVs and causes porcine dermatitis and nephropathy syndrome (PDNS) and reproductive failure in non-PCV2-infected pigs. See Palinski R et al. J Virol. 2016; 91(1): e01879-16. The genomes of PCV3 strains are described in, e.g., Fan S et al. Genome Announc. 2017; 5(15): e00100-17, and Tochetto et al. Transbound Emerg Dis. 2018 February; 65(1):5-9. The genomic sequence of PCV3/CN/Hubei-618/2016 has been deposited at GenBank under the accession number KY354039. Additional strains are recorded under GBANs KX458235, KY996344, and KY96345 (Fux R et al. Virol J. 2018; 15(1):25). Additional aspects of PCV3 biology are described in, e.g., Li G, et al. Adv Sci (Weinh). 2018; 5(9):1800275. Classification of PCVs is still a challenging and evolving field. See, e.g., Fux R et al. Virol J. 2018; 15(1):25. Accordingly, current limits of classification/nomenclature should not limit the scope of PCV-related aspects. In aspects, a PCV3 Ag comprises PCV3a Ag(s), one or more PCV3b Ags, or a combination of at least one of each such type of PCV3 Ag.

In aspects, a PCV3 Ag EP include(s) a PCV3 ORF1 sequence, a PCV3 ORF2 sequence, a fragment of either or both, or a variant of any or all thereof. ORF1 encodes for Rep and Rep′ proteins involved in replication initiation. ORF2 encodes the Cap structural protein. PCV3 Ag EPs can comprise any combination of Rep, Rep′, and Cap sequences. In aspects, a PCV3 Ag EP comprises a Cap protein having a sequence RVRHRSIOI to SEQ ID NO:728, an FF, or a FV of either thereof. In aspects, a PCV3 Ag EP comprises a Rep Ag having a sequence RVRHRSIOI to SEQ ID NO:727, an antigenic fragment thereof, or a variant of either thereof. In aspects, a PCV3 Ag is one of the PCV3 Ags described in US20180305410, e.g., SEQ ID No. 2, 4, 6, or 8 thereof, etc. In aspects, a PCV3 Ag EP comprises SEQ ID NO:6 of the '410 application.

In aspects, a PCV3 Ag also comprises a truncated Rep protein or other PCV Ag homolog, such those that occur in PCV3-CN2018LN-4 (MH277118). See, e.g., Ha Z et al. BMC Vet Res. 2018; 14(1):321. Compared to a highly diverse PCV3 strain (GD2016-1, KY421347), five Vietnamese PCV3 strains contained 39-point nucleotide mutations in the Cap-encoding sequence and 9 of those were non-synonymous. Nguyen V G, Chung H C, Huynh T M L, et al. (2018) Molecular Characterization of Novel Porcine Circovirus 3 (PCV3) in Pig Populations in the North of Vietnam. Arch Gene Genome Res 1(1):24-32. Any variations can be used to design consensus sequence PCV3 variant Ags.

In aspects, one or more PCV3 Ags are deglycosylation site variants. Examples of such variants of a Rep protein, for example, include sequences RVRHROSI to one or more of SEQ ID NOs:724-726.

In aspects, one or more PCV3 Ags are from PCV3/CN/Hubei-618/2016, PCV3/USA/MO2015 (Genbank Accession No. (GBAN) KX778720.1), PCV3/USA/SD2016 (GBAN KX966193.1), PCV3/USA/MN2016 (GBAN KX898030.1), PCV3/USA/29160 (GBAN NC031753.1), PCV3/USA/2164 (GBAN KX458235.1), and PCV3/USA/29160 (GBAN KT869077.1).

In aspects, a PCV3 Ag will also promote, induce, or enhance a detectable or significant response against one or more types of PCV2. In aspects, an EP can comprise one or more PCV2 Ags that detectably or significantly induce an immune response against PCV3. In aspects, one, some, most, or all of the PCV3 Ags in an EP will not induce a significant response against PCV2 or vice versa. A PCV3 Ag EP can comprise any suitable PCV3 Ag(s) from any suitable PCV3 & of any suitable PCV3 PPT, FF, or related PPT.

In aspects, a PCV3 Ag EPESC detectably or significantly induces an immune response against one or more strains of PCV3, such as one or more T cell responses, B cell responses or both. In aspects, a PCV3 Ag EP comprises at least one MHC I epitope, at least one MHC II epitope, or both types of epitopes. In aspects, a PCV3 Ag EP also comprises at least one B cell epitope. In aspects, an effective amount of a PCV3 Ag EPESC detectably or significantly reduces the amount of PCV3 nucleic acid measured in PCV3 infected or challenged pigs (e.g., by qPCR). In aspects, an EA of a PCV3 Ag EPESC DOS reduces the amount of PCV Ag detected in PCV3 infected or challenged pigs, such as by immunohistochemistry (IHC) analysis, ELISA, and the like.

In aspects, an effective amount of a PCV3 Ag EPESC detectably or significantly reduces PCV3-associated porcine dermatitis and nephropathy syndrome (PDNS), reproductive failure, or both. In aspects, an effective amount of a PCV3 Ag EPESC detectably or significantly reduces the number of abortive fetuses in PCV3 infected pigs. In aspects, an effective amount of a PCV3 Ag EPESC detectably or significantly reduces histologic lesions in PCV3 infected or challenged pigs. In aspects, an effective amount of a PCV3 Ag EPESC detectably or significantly reduces viremia, viral shedding, lesions or gross lesions, incidence of swelling (moderate/severe swelling) or discoloration of lymphoid tissues, multisystemic inflammation, myocarditis, or a combination of any or all thereof in PCV3 infected or challenged pigs. In aspects, an effective amount of a PCV3 Ag EPESC detectably or significantly reduces porcine respiratory disease complex (PRDC) or one or more symptoms thereof such as coughing, dyspnea, fever, anorexia, gastro-intestinal disorders (e.g., diarrhea), or tremors in PCV3 challenged or infected pigs. Aspects of some of such phenomena in PCV3 infected pigs are described in, e.g., Klaumann F et al. Front Vet Sci. 2018; 5:315.

In aspects, a PCV3 Ag EPESC is delivered one or more times to piglets as a preventive treatment/vaccine, such as any of the ages described above with respect to PCV2 methods. In aspects, a PCV3 Ag EPESC is delivered as a therapeutic to PCV3 infected pigs. In aspects, a PCV3 Ag EPESC is combined with one or more other PCV3 vaccines or PCV3 treatments, in combined dosage forms or associated treatment methods.

In aspects, a PCV3 Ag EP comprises one or more putative PCV3 CRAs. In aspects, a PCV3 Ag EP comprises one or more CRAs that were established as CRAs by practicing CRA-identification methods provided herein. In aspects, a PCV3 Ag EP comprises one or more heterologous Ags associated with typical PCV3 co-infections. In aspects, a PCV3 Ag EP comprises one or more porcine parvovirus Ags, such as one or more PPV6 Ags, PPV7 Ags, or PPV6 and PPV7 Ags.

2) PRRSV-Porcine Reproductive and Respiratory Syndrome Virus

In aspects, CEPs comprise Ag(s) of, related to, or that induce IR(s) against PRRSV (PRRSV Ag(s)). A PRRSV AgES CEPESC can comprise any suitable number of PRRSV AgES(s) encoding PRRSV Ag(s) from any suitable type of PRRSV and PRRSV PPT(s). In aspects, a PRSSV AgES CEPESC encodes ≥1(1+) or ≥2(2+) PE(s) comprising 2+ PRRSV Ag(s). In aspects, SMGAOA Ag(s) in such PE(s) are bound by linkers (e.g., FLs, MSLs, or MSFLs), associated with/positioned near or adjacent to cleavage sites (e.g., SCS/2A sites), or both. In aspects, OSMGAOA Ag(s) of PRRSV Ag PE(s) are associated with (AW) one or more ITS(s), e.g., PTPS(s), e.g., polyUb sequence(s).

PRRSV Ag CEPs can comprise Ag(s) of or related to any suitable type of PRRSV, any suitable strain of PRRSV, & any suitable PPT. In aspects, a PRRSV Ag EP includes ≥1 type 1 PRRSV Ags, ≥1 type 2 PRRSV Ags, or both. In aspects, an EP comprises one or more Ags of a type 1 PRRSV strain, such as PRRSV-1 Lelystad (GenBank accession #M96262). In aspects, an EP comprises one or more Ags of a type 2 PRRSV strain, such as type 2 PRRSV strain VR-2332 (GenBank accession #AY150564). New PRRSV strains continue to be identified and Ags from such new strains can also be used in PRRSV Ag EPs. See, e.g., Chen N et al. Transbound Emerg Dis. 2019; 66(1):28-34.

In aspects, a PRRSV Ag EP comprises an AARS comprising some, most or all of a PRRSV ORF1a (e.g., comprising some, most, generally all or all of one or more of nsp1α, nsp1β, or nsp2) or an at least related variant sequence. In aspects, a PRRSV Ag EP also comprises an AARS comprising some, most or all of a PRRSV ORF1a′-TF (e.g., comprising some, most, generally all, or all of one or both of nsp2TF or nsp2N) or an at least related variant thereof. In aspects, a PRRSV Ag EP also comprises an AARS comprising some, most or all of a PRRSV ORF1a (e.g., some, most, generally all, or all (SMGAOA) of one or more of nsp3, nsp4, nsp5, nps6, nsp8, nsp7u, and nsp7p). In aspects, a PRRSV Ag EP also comprises an AARS comprising some, most or all of a PRRSV ORF1b (e.g., SMGAOA of one or more of nsp9, nsp1β, nsp11, and nsp12). In aspects, a PRRSV EP also comprises an AARS comprising some, most or all of a PRRSV ORF2a/GP2a sequence. In aspects, a PRRSV Ag EP also comprises an AARS comprising some, most or all of a PRRSV ORF2b/protein E sequence. In aspects, PRRSV Ag(s) comprise SMGAOA of a PRRSV ORF4/GP4 PPT. In aspects, PRRSV Ag EPs comprise SMGAOA of a PRRSV ORF5/GP5 PPT. In aspects, a PRRSV Ag EP also comprises an AARS comprising SMGAOA of a PRRSV ORF5a PPT. In aspects, a PRRSV Ag EP comprises SMGAOA of a PRRSV ORF6/protein M PPT.

In aspects, a PRRSV Ag EP comprises a PRRSV ORF3/GP3 PPT Ag, a PRRSV ORF7/protein N PPT Ag, or a combination thereof, optionally in combination with any of the above-described PRRSV Ag(s). In aspects, a PRRSV Ag EP comprises PRRSV ORF3 Ag(s). In aspects, a PRRSV Ag EP comprises one or more PE(s) (optionally comprising MSLs, FLs, MSFLS, or SCSs or comprising one or more Ag-associated ITSs, e.g., PTPS(s), e.g., polyUb(s). A PRRSV AgES CEPESC can comprise a non-gD PCI, an ICSTAP, or both. In aspects, NAMs of a PRRSV AgES CEPESC are plasmids associated with CaPNPs.

In an exemplary aspect, a PRRSV Ag EP comprises at least one AARS that is at least about 80% or ≥90% identical, such as ≥95%, 97%, or 99% identical to an at least 8 amino acid sequence, at least 12 AARS, at least 15 AARS, or ≥20 AARS contained in SEQ ID NO:11 that induces DOS IR(s) against PRRSV when expressed in TR(s). In aspects, a PRRSV Ag EP comprises ≥50 AAs, ≥100 AAs, or most, generally all, substantially all, or all (MGASAOA) of SEQ ID NO:11.

In aspects, a PRRSV Ag EP comprises a variant Ag AARS. In aspects, a PRRSV AgV EP comprises GSRV(s). In aspects, a PRRSV Ag EP comprises a GSRV of a PRRSV GP2a-b, GP3, GP4, GP5, GP5a, M, or N protein, or CT. In aspects, a PRRSV Ag EP comprises SEQ ID NO:13 or a fragment comprising at least 25% most generally all, of or substantially all thereof. In aspects, a PRRSV Ag EP EP comprises SEQ ID NO:12 or a fragment comprising at least about 10 AAs, ≥20 AAs, ≥25 AAs, ≥50 AAs, most, generally all, or substantially all thereof. In aspects, a PRRSV also comprises a variant in which a decoy epitope (e.g., a decoy epitope in the M protein or GP5) is removed via AA substitution or deletion (See, e.g., U.S. Pat. No. 9,441,015 and aspects described elsewhere).

In aspects, a PRRSV Ag EP comprises one or more PRRSV T cell epitopes (e.g., SEQ ID NO:668); see also CN103242427A). In aspects, a PRRSV Ag EP comprises MHCIE(s), MHCIIE(s), or CT. In aspects, a PRRSV Ag EP also comprises one or more BCE(s) (e.g., SEQ ID NO:669); see also Chen Z et al. J Gen Virol. 2010; 91(Pt 4):1047-1057; An T Q et al. Virus Genes. 2005; 31(1):81-87; de Lima M et al. Virology. 2006; 353(2):410⁻⁴²¹; Oleksiewicz M B et al. J Virol. 2001; 75(7):3277-3290; CN109554375A; and Shi X et al. Int J Biol Macromol. 2019; 139:1288-1294). PRRSV epitopes have been identified and others have been predicted. Any suitable one or more of such PRRSV epitopes can be contained in PRRSV Ag EPs. Additional PRRSV epitopes (including mimotopes), AgES constructs, strains, and methods are KNOWN and adaptable to aspects. SFE Pan X et al Front Immunol. 2020; 10:2995. doi:10.3389/fimmu.2019.02995; WO2007062851; EP1882696; EP2457583; US20120040335; US20110293655; CN102488895; CN103421817; CN105671064; Chen C et al. Vaccine. 2013; 31(14):1838-1847. doi:10.1016/j.vaccine.2013.01.049; U.S. Pat. No. 8,182,984; U.S. Ser. No. 10/300,123; & Oleksiewicz M B et al. J Gen Virol. 2002; 83(Pt 6):1407-1418; R. Parida. Cell-Mediated Immunity in Porcine Reproductive and Respiratory Syndrome Virus Syndrome Virus. Available at digitalcommons.unl.edu; & U.S. Pat. No. 7,465,455.

In aspects, PRRSV Ag CEPs comprises non-PRRSV Ag(s) from/related to DCA(s) that commonly co-infect swine with PRRSV. In aspects, SMGAOA of non-PRRSV Ag(s) in such a CEP are PCV Ag(s). Combined PRRSV/PCV constructs and Ags KNOWN can be adapted to use in such CEPESCs. SFE CN103059142. In aspects, PRRSV AgES CEPESC(s) are delivered to PRRSV/PCV co-infected TR(s) (e.g., pigs) & the method DOS improves one or more virus infection symptoms or conditions in such pigs. Such conditions are described in, e.g., Jung K et al. J Gen Virol. 2009; 90(Pt 11):2713-2723. Additional examples of co-infections DCAs include P. multocida, S.suis, H. parasuis, porcine respiratory coronavirus (PRCV), Mycoplasma hyopneumoniae (M.hyo), and Actinobacillus pleuropneumonia (APP). Methods can comprise administration of two or more NAMs comprising, respectively, PRRSV Ag EPs and one or more such co-infectious DCA Ag EPs or associated administration of such EPESCs (alone or in combination with other vaccines or treatments). The invention also provides CEPESCs comprising one or more Ags against such agents, independently of any PRRSV Ags.

In aspects, a PRRSV Ag EP, PRRSV Ag EPESC, or both, induces one or more DOS IRs against one or more types of PRRSV. In aspects, the IR comprises an enhanced expression of one or more cytokines, such as IFNγ, IL-8, IL-10, TGF-β, or a combination of any or all thereof. In aspects, an IR comprises an increase in proliferation, development, activity, or a COAOAT in one or more other ICs, such as B cells, NK cells, γδT cells early, and αβT cells. In aspects, the IR comprises a T cell immune response. Detection of T cell responses in PRRSV cases are exemplified in, e.g., Bautista E M, et al. Viral Immunol. 1997; 10(2):83-94. In aspects, an IR is an increase in IC frequency, e.g., T cell frequency, of at least 2×, at least 2.5×, or at least 3× over baseline in PRRSV infected or challenged pigs by 2-10 weeks post vaccination. In aspects, the IR comprises a DOS increase in NKC activity, such as NKC cytotoxicity. In aspects, the IR comprises a reduction in TReg suppression of anti-PRRSV responses. In aspects, the IR comprises a detectable or significantly enhanced memory immune response, an upregulation of anti-PRRSV macrophage activity, or both. In aspects, an IR leads to one or more reductions in PRRS frequency, severity, duration, or symptoms or clinical indicators, such as a detectable or significant reduction in shedding, viremia, or both. In aspects, an IR comprises a detectable or significantly lower rate of PRRSV co-infection. Additional relevant principles, etc. (PMCs) are provided in Lunney J K. Annu Rev Anim Biosci. 2016; 4:129-154. In aspects, the delivery of EA(s) of PRRSV CEPESC(s) results in an at least as effective or DOS more effective IR(s) in one or more aspects, CE(s) in 1+ aspect(s) (individually or in a population), or both, with respect to on-market or otherwise described PRRSV vaccines, such as, e.g., FOSTERA® PRRS (Zoetis, USA) or OSMGAOA of the vaccines described in Taeyeon Kim et al. Clinical and Vaccine Immunology May 2015, 22 (6) 631-640 or Oh T, et al. Canadian Journal of Veterinary Research 2019 January; 83(1):57-67. In aspects, a PRRVS EPESC achieves such similar or improved IRs with less administrations than required to achieve such effects using known vaccines.

### 3) Influenza

In aspects, CEPs comprise influenza virus (“IV”) Ag(s). CEPs can comprise any suitable number of IV Ags AW any suitable kind of IV(s) and IV PPT(s). A CEP can comprise IV Ag(s) AW IVs that typically infect 1+ or 2+ species, such as swine influenza virus (SIV), canine influenza virus (CIV), avian influenza virus, or human influenza virus (“HSIV” -the abbreviation HSIV is used for human influenza viruses to avoid confusion with HIV). In aspects, a CEP comprises IV Ags AW IVs that AW with infections of 2+ species, such as dogs & humans; cats & humans; birds & humans; or CT (e.g., FIV Ags, CIV Ags, and HSIV Ags). This principle can also be applied to other viral conditions AW species-to-species transmission in other aspects (e.g., PCV or COV). One aspect provides inhibition/reduction of species-to-species pathogen DCA transmissions, e.g., preventing from NHAs to humans or wild to domestic NHAs.

IV Ag(s) can be from or related to any suitable type or subtype of IV. For example, HSIV Ags can be from or related to type A, type B, or type C HSIVs and can comprise Ags of any subtypes thereof (e.g., H1N1, H1N2, H3N2, or H7N2 HSIV Ags, or H10N8 Ags). In aspects, a CEP comprises multiple IV Ags from a species, multiple IV Ags from multiple species, or both. For example, a CEP can comprise H1N1, H1N2, H3N2, and H7N2 Ags; type A and type B HSIV Ags; or a combination thereof, or further subtypes thereof (e.g., pH1N1). An IV type A or type B Ag can be primarily associated with IVs that infect birds, pigs, horses, cats, dogs, humans, or non-human primates. In aspects an IV Ag CEP comprises an avian IV Ag, such as an H5N1 or H3N2 avian IV Ag. An IV Ag can be from any one of the IVs mentioned in the extensive list of IV strains and serotypes provided in WO2016205347.

In aspects, IV Ag(s) comprise a hemagglutinin (HA or H) AARS(s), such as an H1, H2, H3, H4, H5, H6, H7, H8, H9, H10, H11, H12, H13, H14, H15, H16, H17, or H18 AARS (as always, such AARSs also comprise FFs and FVs, e.g., editope variants, GSRVs, and the like). In aspects, the CEP comprises Ag(s) of or related to a HA head domain (HA1), and optionally lack or comprising a HA cytoplasmic domain, HA transmembrane domain, or a combination. In aspects, CEP(s) comprise Ag(s) from a H7N9 IV, a H10N8 IV, or CT. In aspects, CEPs comprise flu year Ag(s). A “flu year antigen” is an antigen selected from a strain of HSIV used as a component of a yearly flu vaccine (e.g., strain A/Port Chalmers/1/1973(H3N2)-like virus, represents a strain component of the Northern Hemisphere vaccine from 1974-1975).

In aspects, an IV Ag CEP comprises gDAgFP(s). In aspects an IV Ag CEP includes an ICITM(s)/ICSTAP(s), e.g., EAT-2 PPT(s). In aspects, IV Ag CEPs comprise NonCMIP(s), e.g., cytokine(s). In aspects, IV Ag CEPs comprise a NGDCI (e.g., a PD-L1 or CDR112R CI). In aspects, IV Ag CEPs comprise a gDS that is a CI. In aspects, an IV Ag CEP comprises MgDS(s), e.g., in gDAgFP(s). In aspects, EP(s) of IV Ag CEPs is/are encoded on different NAMs. In aspects, an IV Ag CEP comprises ≥2, e.g., 2-20, 3-30, 4-24, 5-25, 5-15, 2-12, 3-12, 4-12, or 5-10 IV Ags. In aspects, an IV Ag CEP comprises PE(s) PCGCOSCOOCO IV Ag(s). In aspects, OSMGAOA of the IV Ags of a CEP are associated with ITS(s), e.g., PTPS(s), e.g., polyUb(s). In aspects, PE(s) comprise(s) linkers (MSLs, FLs, or MSFLs), SCSs (e.g., 2a sites), or CT. In aspects, OSMOA of the Ags of an IV Ag CEP are contained in gDAgFPs.

In aspects, IV Ags of a CEP comprise MHCIE(s), MHCIIE(s), or both, in aspects resulting in DOS CTL IR(s), TH IR(s), and BC IR(s). In aspects, CEP(s) comprise FVs in which BCE(s) are removed or lacks any known IV BCEs. In aspects, the CEP comprises IV BCE(s). In aspects, the CEP comprises DIV AgV(s). In aspects, the CEP comprises a mixture of immunodominant/IRV AVg(s) and subdominant Ag(s). In aspects, a CEP comprises cryptic IV Ag(s).

In aspects, an IV Ag CEP comprises TCE(s) against an internal IV protein, external IV protein, or both. In aspects, an IV Ag CEP comprises MHCIE(s) and MHCIIE(s) against an external IV protein, an internal IV protein, or both. In aspects, a CEP comprises a combination of two, three, or all four of such types of Ags. In aspects, a CEP comprises five or more of such Ags (e.g., 6, 8, 10, 12, 14, 16, or 20 such Ags) in two, three, four of such groupings. Examples of internal and external IV T cell epitopes are exemplified in, e.g., Savic M et al. Immunology. 2016; 147(2):165-177.

In aspects, an IV Ag CEP comprises unnatural immunity Ag(s) (See, e.g., Scorza et al. Vaccine. 2016; 34(26):2926-2933 and Nabel, supra). In aspects, IV Ag(s) comprise IV serotype/strain cross-reactive epitopes. Examples of such epitopes are described in, e.g., Koutsakos M et al. Nat Immunol. 2019; 20(5):613-625. In aspects, a cross-protective IV Ag EP comprises HA stalk Ag(s) or AgV(s).

In aspects, IV Ags comprise IV Ags from IV strains that are predominately from different regions, such as different continents (e.g., Asia, N. America, or Europe) or different hemispheres (Northern and Southern or Eastern or Western). In aspects, IV Ags comprise one or more Ags in the Influenza Sequence and Epitope Database (ISED) (See, e.g., Yang I S, et al. Nucleic Acids Res. 2009; 37. D423-D430.

In aspects, OSMGAOA of any HA IV Ag(s) of a CEP are at least partially glycosylated. In aspects, IV HA AgV(s) comprising GSRV(s) are provided, wherein OSMOA of the N-linked glycosylation sites are removed. In aspects, an HA IV Ag is from a H1, H3, or H5 IV. NLG sites of HA PPTs are exemplified in, e.g., Kim J et al. Yonsei Med J. 2012; 53(5):886-893. In aspects, IV Ag(s) comprise a glycosylated or partially glycosylated IV M2 PPT/AARS. In aspects, IV Ag(S) comprise M2 AgV(s) comprising GSRV(s). NLG sites of M2 PPTs are exemplified in e.g., Holsinger L J et al. J Virol. 1994; 68(3):1551-1563.

In aspects, an IV CEP comprises HA stalk domain (“SD”); matrix protein 1 (M1), matrix protein 2 (M2), or M2e (M2 epitope) Ags; or combinations. In aspects, an IV CEP also comprises IV nucleoprotein (NP) Ag(s) or AgV(s) (e.g., NP Ag(s) and M1 Ag(s); HA SD Ag(s) and M2 Ag(s); or HA SD, M1, M2, and NP Ag(s)). In aspects, a CEP will further comprise a neuraminidase (N) (N1 or N2) Ag. However, in other aspects, a CEP lacks any N Ag(s). In aspects, 2+ such Ag(s), 2+ portions of 1+ of such Ag(s), or both are from different types of IVs. In aspects, one type of such IV Ag is a chimera of HA1 and HA2 sequences, such as a HA1 SD and a HA2 HA head domain Ag (or vice versa). In an aspect, the CEP comprises a combination of 2, 3, or 4+HA Ag(s) comprising HA SD Ag(s), NP Ag(s) (e.g., AgV(s)), M1 Ag(s), and M2 Ag(s), comprising MHCIE(s) & MHCIIE(s), and optionally comprising ITS(s), e.g., polyUb(s). In aspects, IV Ag(s) comprise HA stalk domain AARS(s) Ag(s)/AgV(s); HA head domain AARS(s) Ag(s)/AgV(s), neuraminidase (N) stalk domain Ag(s)/AgV(s), or N head domain Ag(s)/AgV(s). An exemplary HA stalk domain Ag is described in Mallajosyula et al. PNAS (2014) E2514-E2523. IV Ags can PC, GCO, SCO, consist essentially of, or CO such domains, such as being “headless” sequences, soluble HA Ag sequences, and the like. In aspects, IV Ag CEPs comprise anti-IV Ab AARS(s) directed to IV Ag(s). Examples of such Abs are disclosed in US20190062407 & WO202004154. In aspects, such anti-IV Ab sequences form part of a NGDFP. In aspects, NGDFP(s) include IV Ag(s), and optionally ITS(s) (e.g., polyUb(s)).

In aspects, CEPs comprise AgV(s), such as editopes, GSRAgV(s), DIV AgV(s), synthetic AgV(s) (see, e.g, AU2002027676), chimeric AgV(s) (e.g., AgV(s) comprising IV Ag AARS(s) of strains of risk of cross-species transmission), and AgV(s) developed from consensus sequences of IV Ags. In an aspect, a HA loop-forming sequence, such as KRRSNKS (SEQ ID NO:733), is substituted with a GSRV or other variant (e.g., a string of two to eight Gly residues). In aspects, a HA AgV includes a modification that disrupts a site B helix, formed by, e.g., SEQ ID NOs:670 & 671. In aspects, such modifications introduce a glycosylation site, by, e.g., using NAS to replace QIS; substituting NIT for SLY; substituting NST for KYK; or substituting NTS for YKY at 159. CD4 IV Ags include SEQ ID NOs:672-676 (or FFs/FVs). Known CD8 IV epitopes include SEQ ID NOs:677-680 (& FFs/FVs). Examples of known H1N1 epitopes include SEQ ID NOs:681-683 (& FFs/FVs). Additional IV epitopes include SEQ ID NOs:684-691 & FFs/FVs thereof. In aspects, IV CEPs lack epitopes that exhibit a prevalence of more than about 40%, 50%, 60%, 70%, or 80%, but exhibit serotype coverage of less than about 15%, <20%, or ≤˜25% (such principles can be adapted to other viral Ag CEPESCs). In aspects, CEPs comprise CD8 IV epitope(s) identified with the top 25%, 33%, or 50% of IFNg, IL-2, or TNFalpha CD4TC counts, CD8 TC counts, or both in TR(s), e.g., as reported in Savic et al, supra.

In aspects, a CIV Ag CEP comprises ICSTAP(s)/ICITM(s) such as an EAT-2 PPT, SAP PPT, or CT. In aspects, an IV AgES CEP comprises gDS CI(s). In aspects, EP comprise NGDCI(s), e.g., a PD-1/PD-L1 CI or CD112R CI. In aspects, IV Ag(s) are associated with ITS(s), e.g., PTPS(s), e.g., polyUb(s). In aspects, OSMOA of IV Ag(s) are contained in gDAgFP(s) in CEPs. In aspects, CEPES(s) are contained in 2+ NAMs comprising different EPES(s), 1+ of the EPs comprising gDAgFP(s) and a 2^{nd} NAM EP comprising a 2nd gDP, ICSTAP(s)/ICITM(s), NCMIP(s), Ag(s), NGDICRTSAgFPs, or CT.

In aspects, CIV AgES EPECS(s) are contained in viral vectors, such as an Ad vector, MMLV vector, etc. In aspects, such CIV AgES EPEC(s) can be delivered in a non-pathogenic viral vector derived from the DCA or a related virus (i.e., an IV vector). In aspects, CIV AgES CEPES(s) are contained in nucleic acid vectors. In aspects, the CEPNAMs comprise EEI(s). In aspects, the vectors are mRNA vectors. In aspects, the vectors are plasmid DNA vectors associated with a TFA, such as a CaPNP. In aspects, CaPNP(s) of a CaPNP-associated NAV CEPESC exhibit DOS higher hemagglutination inhibition, virus neutralization, anti-IV IgG antibody titers, or CT, than without the CaPNP(s). In aspects, such CEPESCs are administered by mucosal administration, e.g., in NHA TR(s) (e.g., pulmonary, intranasal, intravaginal, or CTs). Such aspects apply to any CaPNP-plasmid aspects of this disclosure and are not limited to IV AgES CEPESCs. In aspects, delivering an effective amount (EA) of a CEPESC comprising IV AgES(s) induces IR(s) against one or more IV(s). In aspects delivering an EA of a CEPESC comprising IV AgES(s) induces DOS CE(s) relating to IV infection, such as DOS reduction in viral shedding, DOS reduction in viremia, or DOS reduction in IV-related nucleic acids in the host. In aspects, an EA of CEPESC comprising IV AgES(s) results in a DOS reduction in IV infection when initially administered more than 48 hours after infection. In aspects, administration of an EA of a CEPESC comprising IV AgES(s) results in a DOS reduction in one or more IV infection-associated symptoms, such as dry cough, fever, chills, myalgias progressing to respiratory failure, and secondary bacterial infections (e.g., MRSA). In aspects, a single administration of an EA of a CEPESC comprising IV AgES(s) provides protection in a significant proportion of a population for an entire flu season or multiple seasons. In aspects, an IV AgES CEPESC induces a DOS improved immunological memory in one or more aspects as compared to one or more IV vaccines in the prior art, such as OSMOA of the on-market IV vaccines (e.g., Fluarix, Fluarix Quadrivalent, FluLaval, Fluzone, or Fluzone Quadrivalent (for HSIV); Nobivac® for CIV; or Ingelvac Provenza™ for SIV. In aspects, the method results in a significant IR against IV in about 14 days or less, ≤˜10 days, or ≤˜7 days. In aspects, inducing anti-IFV IRs by delivering IV AgES CEPESCs comprises administering a booster dose after e.g., 2-4 weeks; 2, 3, or 6 months; or 1, 2, 3, 5, or 10 years (e.g., 2-52 weeks, 1-36 months, or 1⁻⁵ years). In aspects, an IV AgES CEPESC is administered in a CC comprising an anti-IV vaccine or therapeutic. In aspects, an IV AgES CEPESC is administered in association with an anti-IV vaccine/therapy. In aspects, such methods DOS B cell & T cell IRs. IV Ags, epitopes, and other PMCs are provided in U.S. Ser. No. 10/022,435, U.S. Pat. No. 8,470,771, U.S. Ser. No. 10/543,268, U.S. Ser. No. 10/596,250, U.S. Ser. No. 10/584,148, U.S. Ser. No. 10/286,061, US2016020796, US20190275137, US20190321460, US20190134185, US20150086560, US20190201519, WO2016205347, WO2016178811, Gutidrrez A H et al. Influenza Other Respir Viruses. 2017; 11(6):531-542; & Rimmelzwaan G F et al. Vaccine. 2009; 27(45):6363-6365.

In aspects, an IV AgES CEPESC comprises IV Ag(s) of an IV(s) primarily infects NHAs. In aspects, SMGAOA IV Ag(s) of a CEP are Ags of NHA IVs. In aspects, OSMOA of the IV Ags of an EP are CIV Ags. In aspects, a CEP comprises H3N2 CIV Ag(s), H3N8 CIV Ag(s), or CT. In aspects, CEPs comprise MHCIE(s) & MCHIIE(s) against H3N2 or H3N8 or both, or either or both in combination with Ags of/related to PPTs of H1N1, H5N1, H3N1, or CT. In aspects, delivery of an EA of such a CEPESC DOS reduces indicators of CIV in TR(s), such as viral shedding; symptoms of CIV, such as cough, runny nose, fever, lethargy, eye discharge and reduced appetite; or both. In aspects, such a product is delivered to canines with no capability to mount DOS IR(s) to one or more strains of CIV associated with OSMOA of the CIV Ag(s) in the EP. In aspects, CEPESCs express IV Ag(s) AW commercial poultry, dogs, or both. In aspects, CEPESCs include AgES(s) encoding H7N2 IV Ag(s). In aspects, CEPESCs expressing IV Ag(s) exhibit DOS IR(s) on challenge with IV(s) after at least ˜6 months, ≥12 months, ≥18 months, ≥˜2 years, ≥˜3 years, or at least about 5 years. In aspects, treated TRs comprise a DOS amount of Ag-specific T memory cells associated with Ag(s) in the EP. In aspects, a CIV AgES CEP comprises a gDS CI. In aspects, an EP also comprises a NGDCI, such as a PD-1 CI, PD-L1 CI, or a CD112R CI.

In aspects, CIV Ags are associated with ITS(s), such as PTPS(s), e.g., polyUb(s). In aspects, OSMGAOA of such CIV Ags in gDAgFP(s) in the EP. In aspects, CIV Ag CEPs comprise ICSTAP(s)/ITICITM(s) e.g., EAT-2 PPT(s). In aspects, an IV AgES CEP comprises a gDS CI. In aspects, an EP also comprises a NGDCI, such as a PD-1 CI, PD-L1 CI, or a CD112R CI.

In aspects, CIV AgES NAM(s) are NAV(s). In aspects, NAV(s) are mRNA vectors. In aspects, NAV(s) are plasmid DNA vectors associated with a TFA, such as a CaPNP. In aspects, CEPESC NAM(s) comprise EEI(s).

In aspects, CIV AgES CEPES(s) are contained in 2+ NAM(s) comprising different EPESs, where EP(s) comprise gDAgFP(s) and other EP(s) comprise a different gDP, ICSTAP(s), NCI CIV Ag(s), NGDICRTSAgFPs, etc.

In aspects, a CIV Ag EP will comprise CIV HA SD Ag(s), NP Ag(s), M1 Ag(s), M2 Ag(s), or a combination of two, three, or all four thereof (e.g., HA SD/NP/M1/M2 or NP/M1/M2). In aspects, such Ag(s) will comprise MHCI and MHCII Ags. In aspects, OSMOA of such CIV Ag(s) are in gDAgFP(s) of the CEP. In aspects, OSMOA of such Ag(s) are associated with ITS(s), such as polyUb(s). In aspects, the CEP comprises an ITII, such as an EAT-2 PPT. In aspects, the EPs comprise one or more 2A sites, MSLs, FLs, or combinations. In aspects, an EA of any such CEPESC exhibits DOS IR(s) or CE(s) as compared to administration of Nobivac® Canine Flu Bivalent Vaccine. In aspects, delivery of an EA of any such CEPESC results in a DOS increase in IFNgamma in TRs.

### 4) Pseudorabies Virus

In aspects, CEPs comprise pseudorabies virus (PRV) Ag(s). PRV is an alphaherpesvirus and is aka as suid herpesvirus 1 (SuHVI) and Aujeszky's disease virus (ADV) (in reference to a disorder caused by PRV). PRV is interspecies transmissible/zoontic, being indicated infections of other animals such as cows and horses, and recently shown to infect humans. See, e.g., Yang X et al. Int J Infect Dis. 2019; 87:92-99.

PRV AgES CEPESCs can comprise PRV Ag AgES(s) encoding any suitable types of PRV Ag(s) from any suitable type of PRV & PRV PPTs. Numerous PRV strains are KNOWN. See, e.g., Zhai X et al. Virol Sin. 2019; 34(6):601-609; Verpoest S et al. 2014; 172(1-2):72-77; Tong W et al. Vet Microbiol. 2015; 181(3-4):236-240; Tang Y D et al. Sci Rep. 2017; 7(1):7783; Yu X et al. Emerg Infect Dis. 2014; 20(1):102-104; Wu R et al. J Vet Sci. 2013; 14(3):363-365; and Sun Y et al. PeerJ. 2018; 6: e5785. CEPs can comprise PRV Ag(s) from or related to any Ag(s) of such other PRV strain(s).

PRV Ag(s) in CEP(s) can include PRV Ag(s) of or that are related to any suitable PRV PPTs. In aspects, PRV Ag(s) in CEP(s) comprise PRV gB Ag(s), PRV gC Ag(s), PRV gD Ag(s), PRV gE Ag(s) or combinations. In aspects, CEP(s) do not comprise any PRV gD Ag(s). In aspects, PRV gDS(s) in CEP(s) are used to induce gDS functions, such as porcine nectin-1 receptor (e.g., in a PRV gDS or PRV gD-related gDS gDAgFP(s) in the CEP). In aspects, such PRV gDS(s) or PRV-related gDS(s) are the only gDS(s) in the CEP. PRV Ag(s) can include other suitable PRV PPT(s) or AARS(s). Such PPT(s)/AARS(s) and additional relevant aspects of PRV biology that can be adapted or applied to methods/compositions of related aspects of the invention are described in, e.g., Klupp B G et al. J Virol. 2004 February; 78(4):2166 and J Virol. 2004; 78(1):424-440; Ye C et al. Virol J. 2018; 15(1):195. Published 2018 Dec. 29; Pomeranz et al. Microbiol Mol Biol Rev. 2005; 69(3):462-500; Chinsakchai S et al. 1994; 43(1-3):107-116; Stegeman A, et al. Vet Q. 1997; 19(3):117-122; and Pomeranz L E et al. Microbiol Mol Biol Rev. 2005; 69(3):462-500. In aspects, PRV Ag CEP(s) comprise TCE(s). In aspects, PRV Ag CEP(s) comprise MHCIE(s) & MHCIIE(s). In aspects, PRV gC TCE(s) are VRHRSIOI to 1 or both of SEQ ID NOs:692 & 693.

In aspects, PRV-related EP(s) include putative CRA(s) (PCRA(s)).

In aspects, PRV Ag CEP(s) comprise PRV BCE(s). Examples of PRV BCE(s), BC Ag(s), and PRV Ab(s) are KNOWN and adaptable to aspects. See, e.g., Zhang P et al. Vet Microbiol. 2019; 234:83-91; Zaripov M M et al. J Gen Virol 1999 August; 80(Pt 8):2285 and J Gen Virol. 1999; 80 (Pt 3):537-541; Jacobs L et al. J Gen Virol. 1990; 71 (Pt 4):881-887; Jacobs L et al. Clin Diagn Lab Immunol. 1994; 1(5):500-505; Li X et al. PLOS Pathogens 13(12): e1006777; Morenkov O S et al. Virus Res. 1997; 51(1):65-79; Coe N E et al. Arch Virol. 1990; 110(1-2):137-142; and Xu J J et al. Biochem Biophys Res Commun. 2019; 519(2):330-336. In aspects, Abs or Ab AARS(s) from such Ab(s) are also within the CEP. E.g., such Ab AARS(s) can be incorporated into FPs comprising one or more PRV Ag(s) in a CEP or in or expressed by a composition administered in association with PRV Ag CEPESC(s). Additional PRV Ags and related PMCs adaptable to aspects comprising PRV AgES CEPESC(s) are described in, e.g., U.S. Pat. No. 5,449,765; CN107163108; and CN104628865. In aspects, PRV Ag CEPs comprise PRV AgV(s). In aspects, AgV(s) comprise GSRV(s) or DIVs. In aspects, PRV AgV(s) include editope(s) (e.g., improved MHC affinity AgV(s)). In aspects, PRV Ag(s) are associated with ITS(s). In aspects, ITS(s) in the CEP comprise PTPS(s). In aspects, PTPS(s) comprise polyUb(s).

In aspects, a PRV CEP comprises ICSTAP(s)/ITICITM(s). In aspects, ICSTAP(s) comprise EAT-2 PPT(s). In methods, ICITSTAP(s) or ES(s) are AAW delivery of PRV AgES CEPESC(s), e.g., prior to initial CEPESC dosing. In aspects, PRV CEP comprises NGDCI(s). In aspects, NGDCI(s) include PD-L1, PD-1, or CD112R CI(s). In aspects, NGDCI(s) are non-Ab multimeric PPT, such as a trap PPT. In methods, NGDCI(s) are AAW or expressed from NAM(s) delivered in association with PRV AgES CEPESC(s).

In aspects, PRV CEPs comprise NCMIMP(s), such as cytokine(s), e.g., IFNg. In aspects here and with respect to other methods herein SMGAOA NCMIMP(s) are not ICSTAP(s) or ITICITM(s). In other aspects NCMIMP(s) ACA ICSTAP(s)/ITICIMP(s). In methods, NCMIMP(s) are administered or related ES(s) delivered in AW the delivery of an EA of PRV AgES CEPESC(s).

In aspects, CEPs comprise PIM(s) that DOS (a) upregulates an NKG2D ligand (e.g., MULT-1, H60, or RAE-1F); (b) reduces NKG2D inhibition; (c) enhances NKG2D activity; or (d) upregulates NKG2D expression. Aspects can include other modulator(s) of NKG2D activity, NKG2D activity inhibitor(s). In aspects, OSMGAOA of PRV Ag(s) of a CEP are contained in gDAgFP(s). In aspects, OSMGAOA of PRV Ag(s) of a CEP are contained in other FP(s), such as PE FP(s), Ag:ITS FP(s), FP(s) comprising NGDICRTS(s), or combinations of any or all thereof (“CT”).

In aspects, PRV Ag CEsP comprise 2+ gDPs. In aspects, PRV Ag CEPs comprise gDAgFP(s). In aspects, PRV Ag CEP(s) comprise MgDS(s).

In aspects, a PRV Ag CEP comprises gDAgFP(s) comprising gDS(s) that exhibit a higher level of relatedness, similarity, or both to a PRV gD than a human alphaherpesvirus gD. In aspects, gDAgFP(s) comprise a gDS that is RVRHRSOI, SVSHSOCE, or both to ≥1 of SEQ ID NOs:694-696. In aspects, gDS(s) comprise gDS(s) that bind porcine nectin-1 with suitable, comparable, or improved affinity as compared to WT PRV gD, HSV-1 gD, HSV-2 gD, or CT. pNectin-1 binding is described in, e.g., Li A et al. PLoS Pathog. 2017; 13(5): e1006314 and Zago A et al. PNAS 2004; 101(50):17498-17503. In aspects, PRV Ag EPES(s) comprise EEI(s), e.g., CMV Intron A.

In aspects, PRV Ag EPES(s) are contained in NAV(s). In aspects, EPES(s) are contained in viral vector(s), e.g., viral vectors with high transfection rates in 2+ types of TR(s) (e.g., Ad5 vectors for humans and pigs). In aspects, PRV Ag EPES(s) are delivered in a PRV-derived vector (such vectors are described in, e.g., Tan F et al. Methods Mol Biol. 2017; 1581:79-96). In aspects, OSMOA of the vectors are NAV(s). In aspects, NAV(s) comprise mRNA vector(s). In aspects, OSMOA of the vectors are TFA-associated vectors, such as CaPNP-associated plasmids.

In aspects, PRV AgES CEPESC(s) are combined in CC(s) or administered in association with anti-PRV therapeutics, anti-PRV vaccines, or both. Examples of PRV vaccines are described in, e.g., Freuling C M et al. Vaccines against pseudorabies virus (PrV). Vet Microbiol. 2017; 206:3-9. Examples of CCCs used in viral infections, such as PRV infections, include delivery of EA(s) of immunoglobulin, glucocorticoids, antiviral agents, and symptomatic supportive treatments.

In aspects, a PRV AgES CEPESC induces IR(s) in TR(s). In aspects, such IR(s) comprise DOS modulation of NKC activity, such as DOS enhanced MHCII expression, DOS increases in NKC co-stimulatory molecule expression (e.g., CD80/86 expression), or both; DOS enhanced NKC cytotoxicity; or DOS enhanced NKC cytokine production. In aspects, IR(s) comprise DOS proliferation of PRV Ag-specific T cells, such as CD4, CD8, or both CD4 & CD8 T cells. In aspects, IR(s) comprise DOS proliferation of PRV Ag-specific memory T cells. In aspects, IR(s) comprise DOS killing of PRV-infected cells, a reduction in PRV-associated nucleic acids in TR cells/serum, or both. In aspects, IR(s) comprise reduced PRV shedding, reduced PRV Ag in serum/TRs, or both

In aspects, a PRV AgES CEPESC induces DOS anti-PRV CE(s). In aspects, CE(s) include DOS reduction in PRV-associated pregnancy failures (abortions, stillbirths, etc.) or reduced pregnancy/fertility (live offspring) rates. In aspects, CE(s) comprise DOS reduction in fever, coughing, sneezing, anorexia, excess salivation, seizures, constipation, depression, ataxia, circling, pneumonia, and respiratory failure/issues. In aspects, CE(s) comprise reduction in lesions (e.g., gross of focal hepatic, pulmonary, and splenic necrosis and necrotic tonsillitis or reduction of lesions/microscopic lesion in the nervous system). In aspects, CE(s) include reduced meningoencephalitis, ganglioneuritis, and perivascular cuffing by mononuclear cells. In aspects, CE(s) comprise increased piglet survival rates. In aspects, CEPESC(s) are delivered to pigs under 2 months, 6 weeks, or 1 month in age.

### 5) ASFV (African Swine Fever Virus)

In aspects, CEP(s) comprise Ag(s) of or related to African Swine Fever Virus (ASFV). An ASFV Ag CEP can comprise any suitable number of any suitable type of ASFV Ag(s) (e.g., 1, 2, 3, 5, 7, 8, 10, 12, 15, or more ASFV Ag(s)). ASFV Ag(s) can be from any type of ASFV, such as any of ASFV serotypes 1-8, any of the 24 currently known ASFV genotypes, or comprising AARSs encoded by any of the 39 ASFV strains in GenBank, or combinations (See, e.g., Malogolovkin A et al. Emerg Infect Dis. 2015; 21(2):312-315 and Gaudreault N N et al. Vaccines (Basel). 2019; 7(2):56. Published 2019 Jun. 25). ASFV Ag(s) can include Ag(s) from any of the currently known 68 ASFV structural proteins, or approximately 80 nonstructural proteins. In aspects, a CEP comprises ASFV Ag(s) that are cross-protective against two or more types of ASFV, such as two or more ASFV strains within a serotype. ASFV Ag(s) also can be from or be related to any Ag of any of the ASFV strains maintained at the National Research Institute for Veterinary Virology and Microbiology (VNIIVViM) in Pokrov, Russia, many of which are publicly available (See, e.g., Malogolovkin A et al. Emerg Infect Dis. 2015; 21(2):312-315). In aspects, ASFV Ags of a CEP are encoded by ASFV early genes, intermediate genes, late genes, or two or all thereof. Additional ASFV strains, proteins, etc., adaptable to aspects are described in e.g., Alejo A. et al. J Virol 92: e01293-18; Almazon F. et al. 1992. J Virol. November; 66(11):6655-67; de Villers E. et al., 2010. Virology 400 128-136; Gonzales A. et al. 1990 J. Virol 64(5):2073-2081; Yanez R. et al. 1995. Virology. 1995 Apr. 1; 208(1):249-78; Dixon L K et al. Virus Res. 2013; 173(1):3-14; and Farlow J et al. Virol J. 2018; 15(1):190.

In aspects, ASFV Ag(s) comprise TCE(s). In aspects, ASFV Ag(s) comprise MHCIE(s) and MHCIIE(s). In aspects, ASFV Ag(s) comprise BCE(s). In aspects, an ASFV Ag CEP lacks any known ASFV BCE(s). In aspects, ASFV MHCIE(s) and MHCIIE(s) in a CEP DOS BC IR(s). In aspects, ASFV Ag(s) are associated with ITS(s), such as PTPS(s), e.g., polyUb(s). In aspects, ASFV Ag CEP(s) comprise PEs in which OSMOA of the PE Ags are ASFV Ags.

In aspects, ASFV Ag(s) comprise Ag(s) from the proline-rich cytoplasmic domain of CD2v; from regions I and II located within the carboxyl-terminal regions of the C-type lectin protein; or both. Examples of such epitopes include SFLNLTKLYHHHSHY (SEQ ID NO:734); KYNLNRKKSHYTDLL (SEQ ID NO:735), NRKKSHYTDLLFICS (SEQ ID NO:736), SPPPKPCPPPKPCPP (SEQ ID NO:737), and KPCPPPKPCPPPKPC (SEQ ID NO:738) (see, Burmakina et al. J Gen Virol. 2019; 100(2):259-265).

BCE(s) and anti-ASFV Abs that can be applied or adapted to aspects include A151R, B438L, and K205R-A104R (Lokhandwala S et al. PLoS One. 2017; 12(5): e0177007). In aspects, an ASFV Ag CEP comprises anti-ASFV Abs or Ab AARSs, e.g., in NGD FPs comprising anti-ASFV Ab AARSs and ASFV Ags.

In aspects, ASFV Ag(s) are from p49, p72, p30, pp62, p56, p54, j18L, CD2v, p22, p12, or combinations. In aspects, OSMOA ASFV Ag(s) of a CEP are from envelope proteins. In aspects, ASFV Ag(s) comprise Ag(s) of 9GL, p72, ASFV C-type lectin, ASFV helicase, ASFV DNA Pol, KP362L, ASFV DNA ligase, ASFV RNA Pol, A104R, EP364R, F317L, or combinations. In aspects, ASFV Ag(s) comprise p30 AARS(s). In aspects, AASF Ag(s) comprise p54 and p30 Ag(s). In aspects, ASFV Ag(s) in a CEP are limited to 3 types of ASFV Ag(s) or less, e.g., ≤2 types of ASFV Ag(s). In aspects, a CEP lacks ASFV Ag(s) p72, p22, or both. In aspects, ASFV Ag(s) in a CEP comprise A104R, A151R, B119L, B602L, CD2v, K205R, P49 P12, P32, P54, P72, P30/P32, P220, or combinations. In aspects, ASFV Ag(s) of a CEP comprise Ag(s) of CP204L, PK205R, PB602L, CP530R, E183L, PB646L, and combinations. In aspects, an ASFV Ag CEP comprises PE(s) including OSMOA ASFV Ags, such as P32-CD2V-I329L-G6L. Other combinations include CP204L+CP530R, PK205R+E183L, PB602L+PB646L, CP204L+PK205R+PB602L, pp220 (P150-P37-P14-P34), and CP204L+PK205R+CP530R. In aspects, ASFV Ag(s) comprise P11 or P11.5 Ags (e.g., PEERCTYKFNSYTKKMEL (SEQ ID NO:739) and DQEEKKALQNKETKNLGIP (SEQ ID NO:740), respectively). Additional ASFV Ag(s) can comprise Ag(s) from pp62/p62, EP153R, NP1450L, NP419L, MGF405-4R, and MGF360-11L. In aspects, ASFV Ag(s) comprise A151R, CP312R, E146L, E184L, MGF110 type protein Ags, and combinations. Additional ASFV Ags, epitopes, and the like that can be incorporated or adapted for use in CEPESCs are described in, e.g., US20080131449; CN110218732; CN110269932; CN110093356; CN109836478; WO202006040; RU2534343; CN110618279; Netherton C L et al. Front Immunol. 2019; 10:1318; Gaudreault N N, et al. Vaccines (Basel). 2019; 7(2):56.; and Argilaguet J M et al. PLoS One. 2012; 7(9): e40942.

In aspects, an ASFV Ag CEP comprises a sequence that is RVRHRSIOI to SEQ ID NO:697. In aspects, an ASFV Ag CEP comprises PCRA(s) or CRA(s). Application of ELI to ASFV AARS(s) that can be adapted to testing, identification, or confirmation of CRAs in this and other aspects of the invention are described in, e.g., Jenson J. et al. PMID: 10986387 and Lacasta A. et al. 2014. Journal of Virology 88(22)13322-13332.

In aspects, OSMGAOA ASFV Ag(s) are AW ITS(s). In aspects OSMOA ITS(s) are PTPS(s), e.g., polyUb(s). In aspects, ASFV Ag(s) are in PE(s) comprising MSL(s), FL(s), MSFL(s), or SCS(s). In aspects, OSMOA ITS(s) are non-PTPS ITS(s), e.g., ERTPS(s) or exosome ITS(s).

In aspects, an ASFV Ag CEP comprises ASFV AgV(s). In an aspect, ASFV AgV(s) comprises consensus sequence Ags (e.g., developed from consensus of ASFV CRA(s), such as immediate, early, or late ASFV CRA(s)). In aspects, ASFV AgV(s) comprise GSRAgV(s). In aspects, an ASFV Ag CEP comprises a sequence related, very related, highly related, substantially identical, or identical (RVRHRSIOI) to ≥1 of SEQ ID NOs:26-28.

In aspects, ASFV Ag(s) comprise EP402R Ag(s), E248R Ag(s), or both, or FV(s) of one or both thereof (e.g., a GSRAgV). Such PPTs are described in, e.g., Karger A, et al. Viruses. 2019; 11(9):864. doi:10.3390/v11090864 & Alonso, C. et al. 2018, J. General Virology, 99: 613-614 (& Uniprot Refs. Q65200, Q6JHU1, & Q89501).

In aspects, CEP(s) comprise EP240R FV(s) comprising GSRV(s) in the potential NLG sites starting at residue 12, residue 55, residue 75, residue 113, residue 143, or combinations, e.g., the leading N residue being replaced by a D residue. In aspects, CEP(s) comprise EP420R FV(s) comprising GSRV(s) in potential NLG sites starting at residue 25, residue 37, residue 52, residue 55, residue 72, residue 77, residue 81, residue 89, residue 95, residue 108, residue 125, residue 137, residue 148, residue 155, residue 151, residue 363, or combinations, e.g., the leading N residue being replaced by a D residue.

In aspects, an ASFV Ag comprises E248R Ag(s) and 402R Ag(s) (which for clarity's sake can be AgV(s) of either/both thereof). In aspects, the EP402R and E248R Ag(s) are separated by linkers (e.g., MSLs, FLs, or MSFLs), SCS(s) (e.g., 2A site(s)), or both. In aspects, OSMOA of the EP402R/E248R Ags are associated with ITS(s), e.g., PTPS(s), e.g., Ub(s) e.g., SEQ ID NO:1. In aspects, ASFV Ag CEP(s) comprise ITII(s). In aspects, ITII(s) comprise EAT-2 AARS(s). In aspects, AASF Ag CEP(s) comprise NGDCI(s), such as PD-L1 CI(s) or CD112R CI(s). In aspects, OSMOA NGDCI(s) in a CEP lack Ag sequences, are multimeric or both, as in the case of trap proteins. In aspects, ASFV Ag CEP(s) comprise NANCIPIs, such as IFNg.

In aspects, ASFV AgES EPESNAM(s) comprise EEI(s), ISNS(s), or both. In aspects, a CEPESC comprises two or more NAMs comprising different EPESs, such as ESs encoding different gDP(s), a gDAgFP and a NGDICRTSFP, a gDAgFP and an ITII, and the like. In aspects, NAM(s) are NAV(s), such as mRNAs or plasmid DNA(s). In aspects, NAV(s) are associated with TFA(s), such as CaPNP(s). In aspects, EPES(s) comprise SCUP(s). In aspects, ASFV Ag gDAgFP(s) include gDS(s) RHRVRSII to gDS(s) of a human HSV or PRV. In aspects, gDS(s) comprise MgDS(s). In aspects, CEPs comprise gDSS(s) RVRHRSIOI to a WT gDSS of an HSV or PRV. In aspects, OSMOA ASFV Ags in gDAgFP(s) are downstream of any gDS(s) therein. In aspects, gDP(s) of a CEP lack a gDTMD. gDP(s) can include/lack gD PFD(s). In aspects, EPES(s) comprising ASFV AgES(s) comprise NASM(s). In aspects, NASM(s) comprise a FabI gene sequence/triclosan NASM.

In aspects, an ASFV AgES CEPESC comprises another anti-ASFV vaccine or therapeutic. In aspects, an ASFV AgES CEPESC is administered in association with another anti-ASFV vaccine or therapeutic, such as an attenuated ASFV. Examples of such CCC(s) are described in, e.g., Lacasta A. et al. 2015. Vet Res (2015) 46:135; U.S. Ser. No. 10/507,237; US20150165018A1; and Pdrez-Ndnez D et al. Vet Immunol Immunopathol. 2019; 208:34-43.

In aspects, delivery of an EA of an ASFV AgES CEPESC induces anti-ASFV IR(s) in TR(s). In aspects, CEPESC(s) are administered two or more times to TR(s). In aspects, administered CEPESC(s) are identical. In aspects, they are different. In aspects, other compositions are administered in association with CEPESC(s), such as NAMs comprising EPES(s) encoding PIM(s), CI(s), ITII(s), Ag(s), NANCIPI(s), and the like. In aspects, IR(s) comprise DOS increasing the number or activity of one or more IC(s), such as Ag-specific T-cells, BCs, or both; increasing the number or activity of Ag-specific CD4 or CD8 cells; increasing the number or activity of NKCs; increasing the number or activity of TCR-γδ T cells; or a combination thereof. In aspects, IR(s) lead to CE(s), such as DOS reduction in ASFV morbidity or mortality. In aspects, CE(s)/IR(s) comprise DOS reduction in viral shedding, viremia, or CT. In aspects, CE(s) comprise reduced transmission of ASFV, improved memory response(s) to ASFV(s), or both (e.g., after 6, 9, 12, 18, 24, or 36 months). In aspects, CE(s) comprise reduction in one or more ASFV symptoms; such as fever; decreased appetite; weakness; diarrhea; vomiting; coughing; skin reddening, blotching or other discoloration (hyperaemia and cyanosis); and respiratory ailments. In aspects, CE(s) include DOS improved economic indicators for swine (see elsewhere here). In aspects, CE(s) include DOS improved live birth rates, improved rate of survival to adulthood in pigs, or both. Aspects can include reduction in ASFV nucleic acids (e.g., by PCR), antibody titers (e.g., by ELISA measurements), etc. in infected TR(s) or upon challenge. ASFV AgES(s) can be delivered as prophylactics (e.g., in sows, piglets, or both) or therapeutics.

### 6) Equine Herpesvirus

In aspects, CEPESCs comprising equine herpesvirus (EHV) AgES(s) and gDP(s) are provided. Such CEPs can comprise any suitable number of EHV Ag(s) from any suitable type of EHV and EHV PPT(s). In aspects, EHV Ag(s) comprise EHV-1 Ag(s), EHV-4 Ag(s), or both. In aspects, CEP(s) comprise EHV-1 or EHV-4 Ag(s) that are EHV-1 and EHV-4 cross-protective (inducing DOS IR(s) against both types of EHV(s)). EHV Ag(s) can be of or related to any type/subtype or strain of EHV, e.g., an EHV strain exemplified in U.S. Pat. No. 7,323,178. In aspects, OSMOA of the EHV Ag(s) in a CEP are from genes that have homologs in other alphaherpes viruses (e.g., PRV, HSV, GHV, or BHV). In aspects, OSMOA EHV Ag(s) are from or related to the 5 EHV EPs that lack homologs in other alphaherpes viruses (AHVs). In aspects, OSMOA EHV Ag(s) are structural PPTs (e.g., core proteins, nucleocapsid proteins, virion tegument particle proteins, or CT). In aspects, OSMOA of the EHV Ag(s) are EHV envelope proteins. In aspects, OSMOA of the EHV Ag(s) of a CEP are EHV glycoproteins (e.g., gB (gp14), gC (gp13) gD (gp18), gD, gE, gH, gI, gK, gL, gM, gp21/22a, gp10, or combinations). Such PPTs are KNOWN (See, e.g., U.S. Pat. No. 6,193,983). In aspects, CEPs lack any Ag(s) RVRHROSI to gDS(s) of gDP(s), such as gDAgFP(s) of the CEP. In aspects, EHV Ag(s) comprise gH or gC polypeptides (See, e.g., U.S. Pat. No. 6,083,511). In aspects, OSMOA of the EHV Ag(s) are of or are related to EHV gB, gC, or gD Ag(s). In aspects, EHV Ag(s) of CEPs comprise an EHV alpha-TIF Ag (ORF12 Ag). EHV PPTs are KNOWN (See, e.g., Paillot et al. Open Veterinary Science Journal, 2008, 2, 68-91).

In aspects, OSMOA EHV Ag(s) comprise TCE(s), such as MHCIEs or MHCIIEs. In aspects, EHV Ag CEPs comprise both MHCIEs and MHCIIEs. Examples of EHV T-cell Ags are known in the art and such Ag(s) can be adapted to use in EHV AgES(s) of constructs. See, e.g., Soboll G et al. J Gen Virol. 2003; 84(Pt 10):2625-2634 and Kydd, J. H et al. “The immediate early protein of equine herpesvirus-1 (EHV-1) as a target for cytotoxic T lymphocytes in the Thoroughbred horse” at core.ac.uk.

In aspects, EHV Ag(s) comprise EHV immediate early gene ICP4 (ICP4) epitope(s). In aspects, EHV Ag(s) comprise α-TIF (ETIF; VP16-E) epitope(s) (See, e.g., von Einem et al. Journal of Virology 80 (6) 2609-2620). In aspects, EHV Ag(s) comprise ICP4 and α-TIF TCEs. In aspects, EHV Ag(s) comprise TCE(s) of/related to epitopes of α-TIF, ICP0, ICP22, ICP2, etc.

In aspects, EHV Ag(s) of CEPs comprise FV(s) in which 1 BCE(s) in WTC(s) are removed. In aspects, EHV Ag(s) lack any known EHV BCEs.

In aspects, EHV Ag(s) of CEPs comprise one or more EHV BCEs. EHV BC Ags and antibodies are known in the art. See, e.g., Maeda et al. J. Clinical Microbiology March 2004, 42 (3)1095-1098 and WO1994016093. EHV BC Ags/BCEs can be adapted for incorporation in EHV AgES(s) or anti-EHV Abs and Ab AARS(s) can be included in Eps (e.g., NGDFPs).

In aspects, EHV Ag(s) comprise AgV(s). In aspects, OSMOA of EHV Ag(s) are GSRAgV(s). E.g., a GSRV variant of the EHV-1α-TIF (UniProt P28938) can comprise GSRV(s) at the possible NLG sites starting at residue 49, residue 452, or both, e.g., by replacing N AAs at such positions with D residues. In aspects, EHV Ag CEPs comprise CRA(s), PCRA(s), or both. In aspects, EHV Ag CEP comprises Ag-associated ITS(s). In aspects, OSMOA of the ITS(s) are PTPS(s). In aspects, PTPS(s) are polyUb(s). In aspects, EHV Ag CEP(s) comprise ICSTAP(s) or ITICITM(s). In aspects, ITII(s) comprise EAT-2 PPT(s) or EAT-2 AARS(s). In aspects, ITIIES(S) are in a NAM separate from a gDAgFPESNAM.

In aspects, OSMOA of EHV Ag(s) are in PE(s). In aspects EHV Ag PE(s) comprise MSL(s), FL(s), MSFL(s), cleavage sites, or combinations.

In aspects, EHV CEP(s) comprise CI(s). In aspects, gDS(s) of the CEP are CI(s). In aspects, CEPs comprise NGDCI(s). CEPs can include both.

In aspects, EHV Ag CEP(s) comprise NCMIMP(s), e.g., cytokine(s). In aspects, OSMOA of the EHV Ag(s) are contained in gDAgFP(s). In aspects, gDP(s) comprise MgDS(s). In aspects, gDP(s) comprise gDSS(s). In aspects, MgDS(s) comprise gDS(s) with enhanced nectin-1 protein affinity, reduced HVEM binding, or both. In aspects, EHV Ag(s) do not comprise EHV gD Ag(s).

In aspects, gDP(s) comprise gDS(s) that are more RVRHRSIOI to a WT EHV gDS than an HSV gD. In aspects, gDP(s) comprise WT EHV gDS(s), e.g., a sequence RVRHRSIOI to SEQ ID NO:698. In aspects, a WT EHV gD comprises a TMD, intravirion domain, or both, e.g., SEQ ID NO:699. In aspects, either type of gDP or other gDP comprises a sequence that is RVRHRSIOI to an EHV gDSS, such as SEQ ID NO:700.

In aspects, EPES NAM(s) comprise EEI(s), SCUP(s), or both. In aspects, OSMOA EPES(s) are contained in NAV(s), such as mRNA(s) or plasmids. In aspects, EPES(s) are in plasmid(s) associated with TFA(s), such as CaPNP(s). In aspects, an EHV AgES CEPES(s) are contained in two or more NAM(s). In aspects, one type of NAM comprises a gDAgFPES and a second type of NAM in the CEPESC comprises NS(s) encoding different gDAgFP(s), Ag(s), PE(s), ITII(s), NGDCI(s), NANCIPI(s), NGDICRTSAGFP(s) and the like.

In aspects, EHV AgES CEPESC comprise CCC(s), such as EHV vaccines, EHV therapeutics, other immunogenic agents, and the like.

In aspects, delivering EA of an EHV AgES CEPESC to a TR induces DOS IR(s). In aspects, IR(s) comprise DOS proliferation of IC(s), activation of IC(s), or both. In aspects, activated or proliferated ICs comprise T-cells, BCs, NKC(s), or CT. In aspects IR(s) comprise DOS cytokine expression from IC(s).

In aspects, delivering EA of an EHV induces DOS CE(s) in TR(s), such as reduction of EHV shedding, reduction of viremia, reduction of abortion, increase in rate of live births, increase in rate of horses reaching adulthood, reduction in respiratory tract disease (e.g., rhinopharyngitis or tracheobronchitis), a reduction in frequency of EHV-associated ocular diseases (e.g., reduction in EHV associated uveitis or chorioretinal lesions); reduction in neurological lesions (e.g., microlesions); reduction in non-suppurative vasculitis; reduction in EHV spread through a population; reduction in frequency or severity of myeloencephalopathy; or combinations thereof. Aspects of EHV conditions are described in, e.g., Allen G P et al. Equid herpesvirus-1 (EHV-1) and ˜4 (EHV-4) infections. In: Coetzer, J A W and Tustin, RC (Eds.), Infectious Diseases of Livestock. 2nd Ed. Oxford Press: Cape Town; 2004. pp. 829-859 and Oladunni F S et al. Front Microbiol. 2019; 10:2668. In aspects, delivering an EA of an EHV AgES CEPESC induces DOS enhanced memory IR(s) against EHV, such as DOS memory T cell response(s).

### 7) Coronavirus

In aspects, CEPESCs comprising coronavirus (CoV or COV) AgES(s) are provided. CoV Ag CEPs can comprise any suitable number of COV Ag(s) from any suitable type of COV and any suitable type of COV antigens. In aspects, OSMOA of the COV Ag(s) are beta-coronavirus Ag(s), alpha-coronavirus Ag(s), or combinations. In aspects, OSMOA CoV Ag(s) in a CEP are beta-coronavirus Ag(s). In aspects, OSMOA CEP Cov Ag(s) are Embecovirus Ag(s), Hibecovirus Ag(s), Merbecovirus Ag(s), Nobecovirus Ag(s), or Sarbecovirus Ag(s). In aspects, OSMOA CoV Ag(s) are from alpha-COV(s). In aspects, OSMOA COV Ag(s) of CEPs are from a COV that has been identified of posing a substantial risk of zoonosis. In aspects, OSMOA CEP COV Ag(s) are from a strain of COV that (currently) primarily infects NAH(s). In aspects, OSMOA CEP COV Ag(s) are from a bat COV (e.g., a genus Betacoronavirus, subgenus Sarbecovirus CoV, e.g., bat-SL-CoVZC45 or bat-SL-CoVZXC21). In aspects, OSMOA COV Ag(s) are Ag(s) from or related to COV(s) that primarily infect 2 or more species.

In aspects, OSMOA COV Ag(s) in CEPs are from or related to a COV known to infect humans (e.g. HCoV 229E, NL63, OC43, or HKU1). In aspects, OSMOA CEP Cov Ag(s) are HCoV alpha-CoV(s) (e.g., 229E or NL63 COV(s)). In aspects, OSMOA CEP Cov Ag(s) are HCov beta-CoV(s) (e.g., OC43 or HKU1).

In aspects, OSMOA CEP COV Ag(s) are of or related to non-SARS human CoVs. In aspects, OSMOA CEP COV Ag(s) are from “common cold” COV strains (e.g., OC43, or 229E).

In aspects, OSMOA CEP COV Ag(s) are from a SARS-associated CoV (SACOV), a MERS-associated CoV (MACOV) (aka, hCoV-EMC), etc. In aspects, OSMOA CEP Cov Ag(s) are separately from either SACOV(s) or MERCOV(s). In cases OSMOA CoV Ag(s) are Ag(s) from or related to SARS-CoV (identified in 2002, Betacoronavirus, subgenus Sarbecovirus)(COV1), or SARS-CoV-2 (identified 2019 in Wuhan, China) (COV2).

In aspects, OSMOA CEP COV Ag(s) comprise Ag(s) of or related to a COV spike glycoprotein (S), nucleocapsid (N), envelope (E), or membrane glycoprotein (M). In aspects, COV Ag(s), e.g., SACOV Ag(s), comprise ORF3/ORF3a Ag(s), nsp6 Ag(s), or both, in aspects in combinations with N Ag(s), S Ag(s), or M Ag(s). Examples include combinations of S and M Ag(s), S and nsp6 Ag(s), S and ORF3a Ag(s), S and N Ag(s), or S-M-N Ag(s), S-nsp6-ORF3a Ag(S), and other combinations. In aspects, OSMOA of S Ag(s) are from either the S1 portion of S or the S1 portion of S. Certain R-Cov Ag(s) can include Ag(s) against HE (hemagglutinin-esterase (HE)). In aspects, OSMOA Cov Ag(s) are ORF1ab Ag(s). In aspects, OSMOA of Cov Ag(s) are from a defined receptor binding domain (RBD). In aspects, OSMOA of Cov Ag(s) are structural proteins. In aspects, OSMOA of Cov Ag(s) are non-structural proteins. In aspects, COV Ag(s) include both structural/nonstructural Ag(s).

In aspects, a COV Ag comprises most, generally all, or at least substantially all of WT COV PPT(s) (or identified COV PPT domains, such as S1 or S2) or a RVRHROSI FV. A CEP can, e.g., comprise a complete or at least generally complete COV S protein or S domain, such as a SARS COV2 S sequence (e.g., SEQ ID NO: 14 or a HR or SI FV); a complete or at least generally complete SARS COV2 N sequence (e.g., SEQ ID NO: 15 or a HR or SI FV); a complete or at least generally complete COV M protein, such as a SARS COV2 M sequence (e.g., SEQ ID NO: 16 or a HR or SI FV); or combinations.

In aspects, a COV Ag CEP comprises multiple BCEs, multiple TCEs (e.g., multiple MHCIEs and multiple MHCIIEs), or combinations. In aspects, OSMOA of such multiple Ag(s) are in PE(s). In aspects, PE(s) comprise MSL(s), FL(s), MSFL(s), cleavage sites, or combinations. In aspects, SMOA of the COV Ag(s) in a CEP induce Ag-specific IR(s) in a significant amount of, most of, or at least generally all of TR(s). In aspects, immunodominant epitopes, such as immunodominant TCE(S) are modified, excluded, expressed in lower amounts, etc., to ensure SMOA Ag(s) induce IR(s). Such principles apply to any aspect of the invention, not just COV-related CEPESCs or related methods.

Coronavirus proteins are known in the art. For example, a genome of a SARS-CoV-2 is described in, e.g., Lu R et al. Lancet. 2020; 395(10224):565-574. An example of a source of candidate sequences and virus that can be used in development/testing of constructs (e.g., for the selection of PCRA(s) to be included in a CEP) is provided at NCBI Reference Sequence: NC_045512.2. Additional aspects of SARS-Cov2 biology that can be applied to aspects of the invention re described in Fehr A R et al Methods Mol Biol. 2015; 1282:1-23 and Wu A et al. Cell Host Microbe. 2020; 27(3):325-328.

CoV Ag(s) can comprise TCE(s), BCE(s), or both. In aspects, COV Ag(s) comprise MHCIE(s) & MHCIIE(s). In aspects, CoV Ag(s) comprise MHCIE(s), MHCIIE(s), & BCE(s). In aspects, COV BCE(s) induce TR neutralizing Ab production in a significant number of TR(s). In aspects, presence of COV BCE(s) DOS enhances T-cell IR(s) in TR(s) in Cov infected or challenged TR(s).

In aspects, COV TCE(s) in a CEP PCGCOSCOCO TH 1 T-cell epitopes (TH1TCEs). In aspects of developing Cov CRA(s), PCRA(s) are tested for Th1 cytokine responses to select TH1TCE(s). In methods of inducing CoV IR(s) in TR(s) comprising COV AgES CEPESC(s), OSMOA of the TR(s) are tested for Th2 cytokine levels before or during the method and levels of Th2 cytokines above a standard result in (a) administering a different CEPESC (e.g., a CEPESC that induces a lower Th2 response, a greater Th1 response, or both), (b) changing the dosage frequency, amount, etc., of any further CEPESC administrations, (c) temporarily or permanently stopping any CEPESC method, (d) applying any suitable method for reducing or counteracting the Th2 cytokine level, or (e) combinations. Such composition and method aspects can be applied to any type of CEPESC or method aspects (e.g., a CEPESC primarily comprising cancer-related AgES(s) or other pathogen AgES(s)), but such aspects can be very relevant in certain COV infections, e.g., where Th2 cytokine levels have been associated with DOS increased levels of COV-associated fatalities/risks).

In aspects, OSMOA CoV Ag(s) in a CEP induce IR(s) that are cross-reactive against multiple types of CoV(s) (e.g., common cold HCOVs and SACOVs; MERSCOV(s) and SARSCOV(s), bat COV(s) and human COV(s), or SARSCOV1(s) and SARSCOV2(s)). Several predictive cross-reactive epitopes are known, and such knowledge can be adapted to CEPESCs of the invention. Given this fact, CRA methods also can comprise a step of identifying cross-reactive Ag(s) (an aspect that can also apply to other DCA-associated Ag methods—e.g., identifying Ag(s) cross reactive against multiple AHVs).

Specific CoV epitopes have also been experimentally determined or predicted. In aspects, OSMOA of the CoV epitopes are from proven epitopes. In aspects, OSMOA are from predicted epitopes, such as any of the specifically predicted epitopes described in references cited here. For example, Li C K. J Immunol. 2008; 181(8):5490-5500 describes fifty-five T cell epitopes from 128 SARS-CoV1 patients, which can be incorporated herein. Examples include CoV S Ags RVRHRSIOI to SEQ ID NO:701 & ORF3 COV Ag(s) RVRHRSIOI to SEQ ID NO:702. Another SARS Cov epitope expressible in CEPs is SEQ ID NO:703.

SARS-CoV1 (Sars-Cov) Ag(s) known to be functional epitopes that are in identical PPT(s) in SARS-Cov2 include SEQ ID NOs:704-706. These and numerous similar expected cross-reactive epitopes are described in Ahmed S F et al. Viruses. 2020; 12(3):254. Such Ag(s) can be incorporated as PCRA(s) in CEP(s) or used as SARS-CoV2 PCRA(s).

Additional expected/confirmed SARS-Cov2 TCEs that can be incorporated individually or in combination in CEPs include S protein AARs S405-469, S480-499, and S510-521. In aspects, CEPs also include S370-395 and S435-479, which may comprise both TCEs and BCEs. These and more expected epitopes are described in Zhang et al., 2020, Mapping the Immunodominance Landscape of SARS-CoV-2 Spike Protein for the Design of Vaccines against COVID-19, a copy of which is available at biorxiv.org.

Additional predicted SARS-CoV2 epitopes include ITLCFTLKR, which is characterized in Joshi A et al. Inform Med Unlocked. 2020; 19:100338, the numerous predicted epitopes reported in Kiyotani, K., Toyoshima, Y., Nemoto, K. et al. Bioinformatic prediction of potential T cell epitopes for SARS-Cov-2. J Hum Genet (2020); K. M. Kaderi Kibria, e tal. NatureResearch. 2019. DOI: 10.21203/rs.3.rs-21853/vl; Sahoo B et al. International Journal of Applied Biology and Pharmaceutical Technology 11 (2020): 37-45; Bhattacharya, M et al. J Med Virol. 2020; 92: 618-631; and Marek Prachar et al. bioRxiv 2020.03.20.000794 (including AARS(s) common to Cov-MERS/SARS-Cov2 and SARS-CoV1 and SARS-CoV2). Examples of predicted epitopes reported in these references usable as PCRA(s) include SEQ ID NOs:707-715 (and FFs/FVs).

In aspects, OSMOA of COV Ag(s) in a CEP are AgV(s). In aspects, OSMOA AgV(s) comprise GSRV(s) (e.g., CB substitutions or deletions in one, several, or all of the N—X—S sequences or N—X-T AARS motifs present in WTC Ag(s)). In aspects, introducing OOM GSRV(s) is a step in generating PCRA(s). In aspects, OSMOA of COV AgV(s) are editopes. In aspects, OSMOA of COV Ag(s) or a subset thereof, such as BC Ag(s) comprise glycosylation site addition variations. For example, the BCE for SARS-CoV1/CoV2 cross-reactive Ab CR3022 in SARS-Cov2 can be modified to enhance glycosylation sites (e.g., in a manner corresponding to the BCE epitope for CR3022 in SARS-Cov1, adding an NLG site at N protein residue 370), thereby reducing Kd of the Abs for the site to less than 100 nM, ≤50 nM, ≤20 nM, or less than 10 nM. Such an approach is described in Yuan et al. Science 08 May 2020: 630-633. In aspects, consensus sequence PCRA(s) can be developed from corresponding sequences of various COV(s), from identifying differences in highly related sequences of COV(s) that may be associated with different levels of immunogenicity (as exemplified by the CR3022 epitope example), or both. References cited in this section also employ such approaches to propose COV TCEs and BCEs.

In an aspect, COV AgV(s) comprise FV(s) having a sequence that is the same as a full length or generally full length COV PPT sequences.

In an aspect, COV AgV(s) include S PPT AgV(s) according to the formula of SEQ ID No:20), where each X is N/D and least one of X₁-X₂₂ is a D. In aspects, at least 2, ≥3, ≥4, or ≥5 Xs are D AAs. In aspects, most of X₁-X₂₂ are D residues. In aspects, generally all of X₁-X₂₂ (e.g., 90%) are D AAs. In aspects, COV Ag(s) comprise a Cov N PPT AgV(s) according to the formula of SEQ ID NO:21, wherein each of X1-X6 is N/D & ≥1 of X1-X₆ is a D. In aspects, at least 2, ≥3, ≥4, at least 5, or all of X1-X6 are D residues.

In aspects, COV Ag(s) comprise a COV M protein AgV comprising at least the N-terminal 10%, 15%, 25%, or 33% of SEQ ID NO:22.

In aspects, COV Ag CEP(s) comprise ITS(s), e.g., PTPS(s), e.g., polyUb(s). In aspects, ITS(s) comprise non-PTPS ITS(s), e.g., ERTPS(s). In aspects, a COV AgES CEPESC comprises ITIIES(s). In aspects, a CEP comprises ITII(s) comprising EAT-2 PPT(s) or AARS(s) (e.g., hEAT-2, mEAT-2, an FF of either, or a FV). In aspects, ITIIES(s) are on NAM(s) separate from OSMOA of the COV AgES(s) of the CEPESs.

In aspects, a COV Ag CEP comprises CI(s). In aspects, a COV Ag CEP lacks CI(s). In aspects, gDS(s) in the CEP exhibit CI functions in TR(s). In aspects, gD(s) in the CEP do not exhibit CI functions in TR(s). In aspects, the CEP comprises NGDCI(s). In aspects, NGDCI(s) in CEP comprise PD-L1(s), PD-1(s), or CD112R CI(s). In aspects, CEP(s) comprise TOM CI(s). In aspects, CEP(s) comprise gDS CI(s) and NGDCI(s). In aspects, a NGDCI is a multimeric non-Ab sequence CI, such as a trap PPT.

In aspects, a COV Ag CEP comprises NCMIMP(s). In aspects, NCMIMP(s) comprise Th1 cytokine(s). In aspects, Th1 cytokines comprise, PC, or consist of IFNg, IL-2, or both, or FFs/FVs thereof.

In aspects, OSMGAOA COV Ag(s) are contained in gDAgFP(s). In aspects, OSMOA COV Ag(s) are positioned downstream of any gDS(s) in gDAgFP(s). In aspects, some COV Ag(s) are positioned internal to gDSs and some COV Ag(s) are positioned downstream of any gDSs of gDAgFP(s).

In aspects, OSMOA of the gDP(s) of a CEP comprise MgDS(s). In aspects, gDAgFP(S) exhibit DOS reduced HVEM binding, exhibit enhanced nectin-1 binding, or both. In aspects, gDP(s) of a CEP comprise gDSS(s).

In aspects, EPESs are contained in 2+ NAM(s). In aspects, 1 NAM comprises gDAgFPES and 1 NAM comprises NS encoding a different gDAgFP(s), COV Ag(s); ICSTAP(s)/ICITM(s); NGDCI(s); NCMIMP(s); or CT.

In aspects, OSMOA of any NAMs of a CEPESC are NAV(s). In aspects, NAV(s) are mRNA NAV(s). In aspects, NAV(s) are plasmids. In aspects, plasmids are associated with TFA(s), such as CaPNP(s).

In aspects, CEPESCs comprise CCC(s), such as anti-COV vaccines, anti-COV therapeutics, or both. In aspects, CEPESC(s) are AAW such agent(s).

In aspects, the delivery of an EA of CEPESC(s) to TR(s) results in DOS anti-COV IR(s). In aspects, IR(s) comprise reduction in viral shedding, reduction in the amount of virus, reduction in the amount of COV nucleic acids, reduction in COV antibodies, or combinations, in TR(s). In aspects, IR(s) comprise DOS increases in population or activity of NKCs, DCs, T-cells (CD4 or CD8), BCs, or combinations. In aspects, IR(s) comprise one or more T-cell IR(s). Aspects of anti-COV T-cell IR(s) are exemplified in, e.g., Li C K, Wu H, Yan H, et al. T cell responses to whole SARS coronavirus in humans. J Immunol. 2008; 181(8):5490-5500. In aspects, IR(s) comprise DOS in immunological memory for COV(s). In aspects, IR(s) comprise DOS increase in memory T-cells. In aspects, IR(s) comprise DOS increases in COV-associated cytokine production. In aspects, increased cytokine production primarily consists of (PCO), generally consists of (GCO), or consists of (CO) PCOGCOCO Th1 cytokine production.

In aspects, anti-COV IR(s) lead to anti-COV CE(s). In aspects, CE(s) comprise DOS reduction in COV-associated fever, cough, dyspnea, diarrhea, or combinations. In aspects, CE(s) comprise DOS reduced frequency, duration, or severity of COV-associated disease or hospitalization, recurrence, or transmission of COV. In aspects, CE(S) comprise reduced fatality rates in a population of TR(s). In aspects, TR(s) comprise(s) elderly human patients (e.g., at least 65, at least 70, at least 75, or at least 80 years of age), patients with comorbidities (e.g., respiratory illness, obesity, diabetes, hyperlipidemia, coronary artery disease, renal disease, dementia, COPD, cancer, atrial fibrillation, heart disease, heat failure, or combinations. In aspects, TR(s) is/are not fully developed (i.e., children or young NAHs).

In aspects, delivery of COV AgES CEPESCs are repeated TOM times. In aspects, delivery of COV AgES CEPESC(s) is performed in non-infected TR(s) as a prophylactic/vaccination. In aspects, delivery of an effective amount (EA) of COV AgES CEPESC(s) is performed in infected patients as a therapeutic.

### a. Bacterial Ags & Epitopes

In aspects, Ag(s) in a CEP comprise bacterial-associated Ag(s). Examples of such aspects include EPs comprising Ag(s) against Neisseria spp, including N. gonorrhea and N. meningitidis (e.g., transferrin-binding proteins, lactoferrin binding proteins, PilC, or adhesins); S. pyogenes (for example M proteins or fragments thereof), S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin, filamenteous hemagglutinin, adenylate cyclase, or fimbriae), B. parapertussis, B. bronchiseptica; Mycobacterium spp., including M. tuberculosis (for example ESAT6, Antigen 85A, —B or —C, MPT 44, MPT59, MPT45, HSP10, HSP65, HSP70, HSP 75, HSP90, PPD 19 kDa [Rv3763], or PPD 38 kDa [Rv0934]), M. bovis, M. leprae, M. avium, M. paratuberculosis, M. smegmatis; Legionella spp, including L. pneumophila; Escherichia spp, including enterotoxic E. coli (e.g., colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins, and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, or vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin), C. botulinum (for example botulinum toxin), C. difficile (for example clostridium toxins A or B); Bacillus spp., including B. anthracis (for example botulinum toxin); Corynebacterium spp., including C. diphtheriae (for example diphtheria toxin); Borrelia spp., including B. burgdorferi (for example OspA, OspC, DbpA, or DbpB), B. garinii (for example OspA, OspC, DbpA, or DbpB), B. afzelii (for example OspA, OspC, DbpA, or DbpB), B. andersonii (for example OspA, OspC, DbpA, or DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP or heparin-binding proteins), C. pneumoniae (for example MOMP or heparin-binding proteins), C. psittaci; Leptospira spp., including L. interrogans; Treponema spp., including T. pallidum (e.g., rare outer membrane proteins), T. denticola, T. hyodysenteriae; or derived from parasites such as Plasmodium spp., including P. falciparum; Toxoplasma spp., including T. gondii (for example SAG2, SAG3, or Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans. Antigens against M. tuberculosis include Rv2557, Rv2558, RPFs: Rv0837c, Rv1884c, Rv2389c, Rv2450, Rv1009, aceA (Rv0467), PstSl, (Rv0932), SodA (Rv3846), Rv2031c 16kDa1, Tb Ra12, Tb H9, Tb Ra35, Tb38-1, Erd 14, DPV, MTI, MSL, mTTC2 and hTCC1 (WO 99/51748) and fusions thereof, e.g., Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748). Ags against Chlamydia include for example the High Molecular Weight Protein (HWMP) (WO 99/17741), ORF3 (EP 366 412), and putative membrane proteins (Pmps), as well as others described in WO 99/28475. Ags against Streptococcus spp, including S. pneumoniae, include PsaA, PspA, streptolysin, and choline-binding proteins, and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other preferred bacterial vaccines comprise antigens derived from Haemophilus spp., including H. influenzae type B (for example PRP and conjugates thereof), non-typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof. Ag(s) can be Ag(s) of or related to bacteria of any suitable phyla, such as Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

Bacterial Ag(s) can be against Gram positive bacteria, Gram negative bacteria, acid-fast bacteria, or combinations. Gram positive bacteria include Actinomedurae, Actinomyces israelii, Bacillus anthracis, Bacillus cereus, Clostridium botulinum, Clostridium difficile, Clostridium perfringens, Clostridium tetani, Corynebacterium, Enterococcus faecalis, Listeria monocytogenes, Nocardia, Propionibacterium acnes, Staphylococcus aureus, Staphylococcus epiderm, Streptococcus mutans, Streptococcus pneumoniae and the like. Gram negative bacteria include Afipia felis, Bacteroides, Bartonella bacilliformis, Bortadella pertussis, Borrelia burgdorferi, Borrelia recurrentis, Brucella, Calymmatobacterium granulomatis, Campylobacter, Escherichia coli, Francisella tularensis, Gardnerella vaginalis, Haemophilius aegyptius, Haemophilius ducreyi, Haemophilius influenziae, Heliobacter pylori, Legionella pneumophila, Leptospira interrogans, Neisseria meningitidia, Porphyromonas gingivalis, Providencia sturti, Pseudomonas aeruginosa, Salmonella enteridis, Salmonella typhi, Serratia marcescens, Shigella boydii, Streptobacillus moniliformis, Streptococcus pyogenes, Treponema pallidum, Vibrio cholerae, Yersinia enterocolitica, Yersinia pestis and the like. As used herein, acid-fast bacteria include, but are not limited to, Myobacterium avium, Myobacterium leprae, and Myobacterium tuberculosis. Other bacteria include Bartonella henseiae, Chlamydia psittaci, Chlamydia trachomatis, Coxiella burnetii, Mycoplasma pneumoniae, Rickettsia akari, Rickettsia prowazekii, Rickettsia rickettsii, Rickettsia tsutsugamushi, Rickettsia typhi, Ureaplasma urealyticum, Diplococcus pneumoniae, Ehrlichia chafensis, Enterococcus faecium, Meningococci and the like.

Ag(s) can be associated with a baerobic bacterium or an anerobic bacterium and Ag(s) can be associated with autotrophic bacterium or a heterotrophic bacterium. Ag(s) can be associated with a mesophile, a neutrophile, an extremophile, an acidophile, an alkaliphile, a thermophile, a psychrophile, a halophile, or an osmophile.

In aspects, Ag(s) of CEPs induce IR(s) against bacterial pathogens. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis. Bacterial pathogens may also include bacteria that cause resistant bacterial infections, for example, clindamycin-resistant Clostridium difficile, fluoroquinolon-resistant Clostridium difficile, methicillin-resistant Staphylococcus aureus (MRSA), multidrug-resistant Enterococcus faecalis, multidrug-resistant Enterococcus faecium, multidrug-resistance Pseudomonas aeruginosa, multidrug-resistant Acinetobacter baumannii, and vancomycin-resistant Staphylococcus aureus (VRSA). In aspects, Ag(s) of a CEP induce IR(s) against infectious bacteria, such as Mycobacterium tuberculosis, clindamycin-resistant Clostridium difficile, fluoroquinolon-resistant Clostridium difficile, methicillin-resistant Staphylococcus aureus (MRSA), multidrug-resistant Enterococcus faecalis, multidrug-resistant Enterococcus faecium, multidrug-resistance Pseudomonas aeruginosa, multidrug-resistant Acinetobacter baumannii, & vancomycin-resistant S. aureus (VRSA).

In aspects, bacterial Ag(s) in CEPs comprise Ag(s) of or related to PPT(s) of S. pyogenes, Neisseria gonorrhoeae, Acinetobacter baumannii, Klebsiella pneumoniae, Pseudomonas aeruginosa, Burkholderia cepacia, or CT. Bacterial Ag(s) can be Ag(s) of or related to Salmonella, Escherichia, Pseudomonas, Bacillus, Vibrio, Campylobacter, Heliobacter, Erwinia, Borrelia, Pelobacter, Clostridium, Serratia, Xanothomonas, Yersinia, Burkholdia, Listeria, Shigella, Pasteurella, Enterobacter, Corynebacterium, or Streptococcus. Bacterial Ag(s) can be Ag(s) of or related to an anthrax bacterium, an antibiotic-resistant bacterium, a disease-causing bacterium, a food poisoning bacterium, an infectious bacterium, Staphylococcus bacterium, Streptococcus bacterium, or tetanus bacterium. Bacterial Ag(S) can be Ag(s) against mycobacteria, Clostridium tetani, Yersinia pestis, Bacillus anthraces, methicillin-resistant S. aureus (MRSA), Clostridium difficile, or M. tuberculosis. In aspects, Ag(s) in CEP(s) comprise Mycobacterium tuberculosis antigen(s) (e.g., Ag(s) from the Ag85 family of TB Ags, for example, Ag85A and Ag85B; the Esx family of TB antigens, for example, EsxA, EsxB, EsxC, EsxD, EsxE, EsxF, EsxH, EsxO, EsxQ, EsxR, EsxS, EsxT, EsxU, EsxV, & EsxW; or CT).

In aspects, CEPs comprise Ag(s) against bacteria that primarily infect NAH(s) (e.g., companion animals, such as dogs, horses, or cats; livestock animals, such as pigs, cows, and sheep; or both). In aspects, CEPs comprise bacterial Ag(s) from bacteria that primarily infect humans. In aspects, CEPs comprise bacterial Ag(s) from bacteria that infect both humans and NHA(s) or that are considered at risk for zoonosis.

In aspects, CEPs comprise bacterial Ag(s) that are T-cell Ag(s), B-cell Ag(s), or both. In aspects, CEPs comprise anti-bacterial MHCIE(S), MHCIIE(s), or both. CEP(s) can comprise bacterial PPT PCRA(s), CRA(s), known bacterial Ag(S)/epitope(s), or combinations. Numerous epitopes for bacterial diseases are known in the art, including AgV(s). For example, known and variant M. tuberculosis TCEs that can be adapted for or incorporated into CEP(s) are described in Coscolla M et al. Cell Host Microbe. 2015; 18(5):538-548. S. aureus TCE(s) that can be used in CEPs are described in, e.g., Yang S et al. Microb Pathog. 2020; 144:104167; Ooi J D et al. Nat Commun. 2019; 10(1):3392; and Yu S et al. Microb Pathog. 2015; 89:108-113. Epitopes and identification of TCEs in Pseudomonas aeruginosa are exemplified in, e.g., Parmely M J et al. J Exp Med. 1984; 160(5):1338-1349 and Worgall S, Krause A, Rivara M, et al. Protection against P. aeruginosa with an adenovirus vector containing an OprF epitope in the capsid. J Clin Invest. 2005; 115(5):1281-1289 and related epitope prediction methods are described in Elhag et al. bioRxiv 720730; Clostridium difficile Ags are described in, e.g., Broecker F et al. Nat Commun. 2016; 7:11224 & Seregin S S, et al. Vaccine. 2012; 30(8):1492-1501.

In aspects, OSMOA of bacterial Ag(s) in CEPs are AgV(s) (e.g., editope(s)). In aspects, OSMOA AgV(s) comprise GSRV(s), other DIV(s), or CT. In aspects, CEP(s) comprise bacterial PPT PCRA(s), CRA(s), or both.

In aspects, CEP(s) comprise multiple bacterial Ag(s). In aspects, multiple bacterial Ag(s) are from the same species, type, or strain of bacteria. In aspects, multiple Ag(s) are from different species, types, or strains of bacteria. In aspects, OSMOA of the bacterial Ag(s) are in PE(s). In aspects, PE(s) comprise MSL(s), FL(s), MSFL(s), cleavage sites (e.g., 2A sites), or combinations, in association with OSMOA of the Ag(s) in PE(s). In aspects, OSMOA bacterial Ag(s) in a CEP are in gDAgFP(s).

In aspects, OSMOA bacterial Ag(s) in CEPs are AW ITS(s), such as PTPS(s). In aspects, OSMOA bacterial Ag(s) are associated with polyUb(s). In aspects, a bacterial Ag CEP comprises ITII(s). In aspects, ITII(s) comprise EAT-2 PPT(s) or EAT-2 AARS(S) (e.g., EAT-2 FPs). In aspects, ITII(s) comprise hEAT-2, mEAT-2, FFs of either thereof, or FV of any thereof. In aspects, a bacterial Ag CEP comprises CI(s). In aspects, gDS(s) act as CI(s) in TR(s). In aspects, a CEP comprises NGDCI(s). In aspects, a CEP comprises NGDCI(s) and gDS(s) that are CI(s) in TR(s). In aspects, NGDCI(s) comprise PD-L1 or CD112R CI(s). In aspects, CI(s) comprise multimeric non-Ab AAR Cis, such as trap proteins.

In aspects, a bacterial Ag CEP comprises NANCIPI(s), e.g., cytokine(s). In aspects, most or all NANCIPI(s) are Th1 cytokines.

In aspects, a bacterial Ag CEP comprises a gDAgFP comprising MgDS(s). In aspects, bacterial Ag CEP(s) comprise multiple gDP(s). In aspects, bacterial Ag CEPs comprise multiple bacterial Ag gDAgFPs. In aspects, OSMOA gDP(s) in such a CEP comprise gDSS(s). In aspects, bacterial Ag CEPs comprise NGDICRTSFPs comprising bacterial Ag(s). In aspects, bacterial AgES CEPESCs include EEI(s), ISNS(s), or CT.

In aspects, a bacterial AgES CEPESC comprises multiple NAMs. In aspects, a first NAM encodes a gDAgFP and a second NAM encodes a different gDP, bacterial Ag(s) (e.g., PE(s)), NGDICRTSFP(s), ITII(s), CI(s) (e.g., NGDCI(s)), NANCIPI(s), or combinations.

An example of a multiple-NAM composition is a composition comprising ≥2 NAMs, wherein each of the ≥2 NAMs comprise ≥1 NS not contained in at least one other of the ≥2 NAMs, at least one of the NAM(s) comprising an NS that encodes different EP(s) from at least one other NAM in the composition, at least one of the at least two nucleic acid molecules comprising a nucleotide sequence encoding one or more Ag(s) against a DCA, and at least one of the at least two nucleic acid molecules does not encode any antigens that induce immune responses against the disease-causing agent.

In aspects, OSMOA of the NAM(s) of a bacterial AgES CEPESC are NAV(s), such as mRNA(s) or plasmid(s). In aspects, plasmid(s) are associated with TFA(s), such as CaPNP(s).

In aspects, bacterial AgES CEPESC(s) are combined with CCC(s), such as antibiotics, NAM(s) encoding anti-bacterial compositions, anti-bacterial peptides, etc. In aspects, methods of delivering such CEPESC(s) comprise administering one or more anti-bacterial agents (vaccines, antibiotics or other anti-bacterial therapeutics, etc.) to TR(s).

In aspects, delivery of EA(s) of bacterial AgES CEPESC(s) induce anti-bacterial IR(s) in TR(s). In aspects, IR(s) include reduction in bacterial Abs, bacterial NS(s) (e.g., as determined by qPCR), or both. In aspects, IR(s) comprise increase in the number or activity of IC(s), such as anti-bacterial T-cells, BCs, NKCs, APCs, and the like. In aspects, IR(s) lead to CE(s) such as the treatment of disorders, diseases, or conditions associated with bacterial infection including DOS reduction in abscesses, actinomycosis, acute prostatitis, Aeromonas hydrophila, annual ryegrass toxicity, anthrax, bacillary peliosis, bacteremia, bacterial gastroenteritis, bacterial meningitis, bacterial pneumonia, bacterial vaginosis, bacterium-related cutaneous conditions, bartonellosis, BCG-oma, botryomycosis, botulism, Brazilian purpuric fever, Brodie abscess, brucellosis, Buruli ulcer, campylobacteriosis, caries, Carrion's disease, cat scratch disease, cellulitis, chlamydia infection, cholera, chronic bacterial prostatitis, chronic recurrent multifocal osteomyelitis, clostridial necrotizing enteritis, combined periodontic-endodontic lesions, contagious bovine pleuropneumonia, diphtheria, diphtheritic stomatitis, ehrlichiosis, erysipelas, piglottitis, erysipelas, Fitz-Hugh-Curtis syndrome, flea-borne spotted fever, foot rot (infectious pododermatitis), Garre's sclerosing osteomyelitis, Gonorrhea, Granuloma inguinale, human granulocytic anaplasmosis, human monocytotropic ehrlichiosis, hundred days' cough, impetigo, late congenital syphilitic oculopathy, legionellosis, Lemierre's syndrome, leprosy (Hansen's Disease), leptospirosis, listeriosis, Lyme disease, lymphadenitis, melioidosis, meningococcal disease, meningococcal septicaemia, methicillin-resistant Staphylococcus aureus (MRSA) infection, Mycobacterium avium-intracellulare (MAI), mycoplasma pneumonia, necrotizing fasciitis, nocardiosis, noma (cancrum oris or gangrenous stomatitis), omphalitis, orbital cellulitis, osteomyelitis, overwhelming post-splenectomy infection (OPSI), ovine brucellosis, pasteurellosis, periorbital cellulitis, pertussis (whooping cough), plague, pneumococcal pneumonia, Pott disease, proctitis, pseudomonas infection, psittacosis, pyaemia, pyomyositis, Q fever, relapsing fever (typhinia), rheumatic fever, Rocky Mountain spotted fever (RMSF), rickettsiosis, salmonellosis, scarlet fever, sepsis, serratia infection, shigellosis, southern tick-associated rash illness, staphylococcal scalded skin syndrome, streptococcal pharyngitis, swimming pool granuloma, swine brucellosis, syphilis, syphilitic aortitis, tetanus, toxic shock syndrome (TSS), trachoma, trench fever, tropical ulcer, tuberculosis, tularemia, typhoid fever, typhus, urogenital tuberculosis, urinary tract infections, vancomycin-resistant Staphylococcus aureus infection, Waterhouse-Friderichsen syndrome, pseudotuberculosis (Yersinia) disease, yersiniosis, and combinations. In aspects, delivery of bacterial AgES CEPESCs results in a DOS enhanced IR, CE, or both (e.g., improved memory IR(s)) over existing anti-bacterial vaccines, such as peptide anti-bacterial vaccines.

### b. NVNBO Ags & Epitopes

In aspects, an EP comprises Ag(s) against a non-viral, non-bacterial pathogenic organism (NVNBO), which in aspects are microorganisms (NVNBMs). In aspects, NVNBOs comprise parasitic DCA(s) (parasite(s)). A parasite against which such Ag(s) can be directed can be, e.g., a protozoon, helminth, or ectoparasite. A helminth can be a flatworm (e.g., flukes and tapeworms), a thorny-headed worm, or a round worm (e.g., pinworms). Ectoparasites include lice, fleas, ticks, and mites. Pathogenic protozoans and helminths infections against which Ag(s) can be directed include: amebiasis; malaria; leishmaniasis; trypanosomiasis; toxoplasmosis; Pneumocystis carinii; babesiosis; giardiasis; trichinosis; filariasis; schistosomiasis; nematodes; trematodes or flukes; and cestode (tapeworm) infections. In aspects, parasite Ag(s) are associated with OOM of Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis. In aspects, parasite Ag(s) are associated with one or more of Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus-lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti. In aspects, Ag(s) comprise parasite antigen(s) of or related to PPT(s) expressed in Babesia, Entomoeba, Leishmania, Plasmodium, Trypanosoma, Toxoplasma, Giarda, flat worms/round worms.

In aspects, CEP(s) comprise Ag(s) of/related to Ag(s) of protozoa, e.g., Entamoeba histolytica, Giardia lambila, Trichomonas vaginalis, Trypanosoma brucei, T. cruzi, L. donovani, Balantidium coli, Toxoplasma gondii, Plasmodium spp., & Babesia microti. In aspects, CEP(s) include Ag(s) of/related to parasites, e.g., Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, P. falciparum, Schistosoma, Strongyloides stercoralis, mite, tapeworm, Toxoplasma gondii, Trypanosoma, whipworm, & Wuchereria bancrofti.

In aspects, CEP(s) comprise Ag(s) against a species of the genus Leishmania. In aspects, Leishmania Ag(s) comprise AARS(s) of or related to PPT(s) of an L. donovani complex Leishmania (e.g., an L. donovani or L. infantum (L. chagasi) PPT); an L. mexicana complex Leishmania (e.g., a L. mexicana, L. amazonensis, or L. venezuelensis PPT); L. tropica; L. major; L. aethiopica; or a member of the subgenus Viannia (e.g., a PPT of L. braziliensis, L. guyanensis, L. panamensis, or L. peruviana). In aspects, Leishmania Ag(s) are cross-protective against TOM Leishmania species, types, or strains.

In aspects, Leishmania Ag(s) comprise AARS(s) of or related to antigenic Leishmania PPTs such as gp63, H1, NH36, LACK, Leishmania PSA, Leishmania H2B, LmIRAB, P0, KMP-11, A2, HSP, HSC, LmSTI-1, CPa, CPb. HASP, LPG, CPc, elongation factor-2 (elF-2), enolase, aldolase, triose phosphate isomerase (TPI), protein disulfide isomerase (PDI), or p45. In aspects, Leishmania Ag(s) comprise AARS(s) from or that are related to AARS(s) of Leishmania LACK, NH36, KMP-11, gP63, LmIRAB, H2B, CPa, or Cpb PPTs or combinations. Such PPTs are described in, e.g., Sundar S, et al. Expert Rev Vaccines. 2014; 13(4):489-505. Known and predicted Ags and epitopes against Leishmania species are known in the art and described in E Silva R F et al. Front Immunol. 2020; 10:3145; Zhang J et al. PLoS One. 2020; 15(3): e0230381; Palatnik-de-Sousa C B. Front Immunol. 2019; 10:813; Hamrouni S et al. PLoS Negl Trop Dis. 2020; 14(3): e0008093; jaya Kumar et al., Front. Immunol., 2017; Das et al., Science Translational Medicine, 2014: 234RA56; Kashyap M et al. Infect Genet Evol. 2017; 53:107-115; Alves-Silva M V et al. Front Immunol. 2017; 8:100. In aspects, OSMOA of the Leishmania epitope(s) are THIEs. Examples of such epitopes are known in the art (See, e.g., Joshi S et al. Front Immunol. 2019; 10:288). Such Ag(s)/epitopes can be adapted for AgES(s) or can be modified to form AgV(s), PE(s), and the like, or associated with ITS(s), such as polyUb(s), or both. In aspects, CEPESCs comprise PCRA(s) or CRA(s) related to NVNBOs, such as Leishmania. Additional PMCs are provided in Bordbar A et al. Infect Genet Evol. 2020; 80:104189.

In aspects, delivery of EA(s) of Leishmania AgES CEPESCs result in anti-Leishmania IR(s). In aspects, such IR(s) include DOS increases in the production of IL-12, IFN-γ, or both. In aspects, IR(s) comprise an increase in the number or activity of IC(s), such as Leishmania Ag-specific T-cells, B cells, or both. In aspects, IR(s) comprise increased anti-Leishmania innate immune cell or innate trained immune cell activity (e.g., enhanced macrophage activity). In aspects, IR(s) include enhanced anti-Leishmania memory immune responses, such as an increase in the number of memory T cells specific for Leishmania Ag(s) (e.g., DOS increases in Leishmania Ag-specific CD4+ and CD8+ central memory T cells). In aspects, IR(s) lead to DOS CE(s) such as reduction in lesions, reduction in visceral disease, reduction in Leishmania-related fatalities, and reduction of Leishmania-related symptoms, such as weight loss, enlarged spleen or liver, prolonged fever, and the like.

In aspects, CEPs comprise Ag(s) against malaria-associated DCA Ags, e.g., Plasmodia falciparum PPTs, such as RTS, S, and TRAP (See, e.g., WO9310152). Other plasmodia Ags that can be in CEPs include P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, or Pfs230 PPTs/AARSs and their analogues in Plasmodium spp. The Plasmodium falciparum Ag can include the circumsporozoite (CS) antigen. In aspects, Ag(s) can include P. falciparum PPTs CS, LSA1, TRAP, CelTOS (Ag2), and Amal. In aspects, Ag(s) comprise CSP PPT(s) or FF/FV thereof. Such PPTs are described in U.S. Pat. No. 8,470,560. In aspects, such Ag(s) (or other anti-pathogen Ag(s)) are cross-reactive against 2+ pathogen strain/species.

In cases delivery of an EA of CEPESC(s) DOS induces IR(s), CE(s), or both against NVNBO(s) in TR(s). In aspects, such methods treat or treat the symptoms of diseases such as amoebiasis, giardiasis, trichomoniasis, African Sleeping Sickness, American Sleeping Sickness, leishmaniasis, balantidiasis, toxoplasmosis, malaria, acanthamoeba keratitis, & babesiosis. In aspects, such methods treat diseases or conditions associated with parasitic infections, e.g., acanthamoeba keratitis, amoebiasis, ascariasis, babesiosis, balantidiasis, baylisascariasis, chagas disease, clonorchiasis, cochliomyia, cryptosporidiosis, diphyllobothriasis, dracunculiasis, echinococcosis, elephantiasis, enterobiasis, fascioliasis, fasciolopsiasis, filariasis, giardiasis, gnathostomiasis, hymenolepiasis, isosporiasis, katayama fever, leishmaniasis, lyme disease, malaria, metagonimiasis, myiasis, onchocerciasis, pediculosis, scabies, schistosomiasis, sleeping sickness, strongyloidiasis, taeniasis, toxocariasis, toxoplasmosis, trichinosis, & trichuriasis.

In aspects, a CEP comprises fungi-associated Ag(s) (fungal Ag(s)). In aspects, a CEP comprises Ag(s) associated with or related to PPTs of a fungus of an Aspergillus species, Blastomyces dermatitides, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium. In aspects, CEP(s) comprise Ag(s) that are of or related to PPT(s)/AARS(s) expressed in Aspergillus, Coccidoides, Cryptococcus, Coccidioides immitis, Candida Nocardia, Candida albican, Pneumocystis, and Chlamydia. In aspects, Ag(s) comprise AARS(s) of or related to PPT(s) expressed in Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera). In aspects, CEPs comprise Ag(s) associated with dermatophytic fungi or keratinophilic fungi. In aspects, TCE(s) in such CEPs CPCGCOSCOCO TH17Es. In aspects, CEPESCs comprising fungal Ag(s) induce anti-fungal IRs, such as DOS increase in the number/activity of anti-fungal-specific T-cells or BCs or DOS proliferation or activity enhancement in ITICs, such as DCs, NKCs, or CT. In aspects, such IR(s) result in CE(s), such as DOS reduction in fungal spores, or DOS reduction in the cause or conditions of associated fungal infections, such as aspergilloses, blastomycosis, candidasis, coccidioidomycosis, cryptococcosis, histoplasmosis, mycetomas, paracoccidioidomycosis, and tinea pedis. Anti-fungal CEPESCs can comprise any components of CEPESCs described herein, such as ITII(s), CI(s) (including NGDCI(s)), NANCIPI(s) and the like. ES(s) can comprise EEI(s) and can be in TFA-associated NAVs.

### ii. Cancer Ags & Epitopes

In a further aspect, CEPESCs comprising cancer AgES(s) are provided. Numerous aspects of cancer are described here and the methods-related portions of this Detailed Description, infra. In general, any of the aspects, principles, and methods described in either section or in any other related section of this disclosure can be applied to the practice of the invention (e.g., incorporated in CEPESC methods or compositions).

Cancer AgES CEPESCs can comprise any suitable number of cancer AgES(s) from any suitable type of cancer(s). Cancer and cancer classification is described further in US20200325182.

In aspects, cancer Ag(s) (or “cAg(s)”) in CEPs are from or related to C1-LUAD-enriched cancer cell PPTs; C2-Squamous-like cancer cell PPTs; C3-BRCA/Luminal cancer cell PPTs; C4-BRCA/Basal cancer cell PPTs; C5-KIRC cancer cell PPTs; C6-UCEC cancer cell PPTs; C7-COAD/READ cancer cell PPTs; C8-BLCA cancer cell PPTs; C9-OV cancer cell PPTs; C10-GBM cancer cell PPTs; or C13-AML cancer PPTs; or combinations. Relevant molecular characterization of such cells is described in, e.g., Hoadley K A et al. Cell. 2014; 158(4):929-944.

In aspects, cAg(s) in CEPs are from cancer cells immunologically classified as type C1 cancer cells (e.g., occurring frequently in colorectal cancer, lung squamous cell carcinomas, breast carcinoma, head and neck carcinoma and chromosomally unstable gastrointestinal cancer); type C2 (IFN-γ dominant) cancer cells (associated frequently with gastric, ovarian (OV), HNSC, and cervical tumors (CESC)); type C3 (Inflammatory) cancer cells, associated frequently with kidney, prostate, and pancreatic cancers, and papillary thyroid carcinomas; type C4 (Lymphocyte Depleted) cancer cells (associated with adrenocortical carcinoma (ACC), pheochromocytoma and paraganglioma (PCPG), hepatocellular carcinoma (LIHC), and gliomas) type C5 (Immunologically Quiet) cancer cells frequently associated with lower grade gliomas (LGGs); type C6 (TGF-β Dominant) cancer cells; and CT. Such immunological classification is described in Thorsson V et al. 2019 20; 51(2):411-412 & Immunity. 2018; 48(4):812-830.e14.

In aspects, cAg(s) are associated (are from/related to and induce IR(s) against) carcinoma cells/cancers (e.g., adenocarcinoma, basal cell carcinoma, squamous cell carcinoma, & transitional cell carcinoma). In aspects, cAg(s) are associated with sarcoma cells/cancers. In aspects, cAg(s) are associated with leukemia (e.g., lymphoblastic leukemia). In cases, cAg(s) are AW a myelodysplastic syndrome cancer (e.g., acute myeloid leukemia (AML)). In cases CAg(s) are AW choriocarcinoma, chronic lymphocytic leukemia, chronic myeloid leukemia, Hodgkin lymphoma, myeloma, melanoma, mesothelioma, non-Hodgkin lymphoma (NHL), soft tissue sarcoma or other sarcoma (e.g., an angiosarcoma or chondrosarcoma), glioblastoma, neuroblastoma, glioma, blastoma, gestational trophoblastic tumour, blastic plasmacytoid dendritic cell neoplasm, acral lentiginous melanoma, actinic keratoses, acute lymphocytic leukemia, adenoid cystic carcinoma, adenomas, anorectum cancer, astrocytic tumor, bartholin gland carcinoma, bronchial gland carcinoma, carcinoid, cholangiocarcinoma, chondosarcoma, choriod plexus papilloma/carcinoma, chronic lymphocytic leukemia, clear cell carcinoma, cystadenoma, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gastric antrum cancer, gastric fundus cancer, gastrinoma, glioblastoma, glucagonoma, hemangiblastomas, hemangioendothelioma, hemangiomas, hepatic adenoma, hepatic adenomatosis, hepatobiliary cancer, hepatocellular carcinoma, insulinoma, intaepithelial neoplasia, interepithelial squamous cell neoplasia, intrahepatic bile duct cancer, jejunum cancer, Kaposi's sarcoma, leiomyosarcoma, lentigo malignant melanomas, lymphoma, male genital cancer, malignant melanoma, malignant mesothelial tumors, medulloblastoma, medulloepithelioma, meningeal cancer, mesothelial cancer, metastatic carcinoma, mucoepidermoid carcinoma, multiple myeloma, neuroepithelial adenocarcinoma nodular melanoma, non-epithelial skin cancer, non-Hodgkin's lymphoma, oat cell carcinoma, oligodendroglial cancer, osteosarcoma, papillary serous adenocarcinoma, plasmacytoma, pseudosarcoma, pulmonary blastoma, renal cell carcinoma, retinoblastoma, rhabdomyosarcoma, sarcoma, small cell carcinoma, serous carcinoma, somatostatin-secreting tumor, submesothelial cancer, superficial spreading melanoma, T cell leukemia, verrucous carcinoma, VIPoma, uterine cervix cancer, uterine corpus cancer, uveal melanoma, Wilms tumor, or hairy cell leukemia.

In aspects, cAg(s) are associated with cells of anal cancer (including anal canal cancer), bile duct cancer, bilary tract cancer, bladder cancer, bone cancer, bone marrow cancer, bowel cancer, brain tumor, breast cancer, cervical cancer, CNS cancer, colon cancer, connective tissue cancer, colorectal cancer, colon cancer, endometrial cancer, eye cancer, digestive system cancer, duodenum cancer, endocrine system cancer, gallbladder cancer, gastric cancer, glial cancer, head and neck cancer, heart cancer, ileum cancer, joint cancer, kidney cancer, large intestine cancer, laryngeal cancer, liver cancer, lung cancer (e.g., small-cell lung cancer or non-small cell lung cancer), mouth or oropharyngeal cancer, muscle cancer, nasal and sinus cancers, nasopharyngeal cancer, nasal tract cancer, esophageal cancer, ovarian cancer, pancreatic cancer, parotid cancer, penile cancer, pituitary cancer, prostate cancer, pharynx cancer, renal cancer, rectal cancer, respiratory system cancer, salivary gland cancer, skin cancer (e.g., non-melanoma skin cancer), sinus cancer, small intestine cancer, stomach cancer, testicular cancer, tongue cancer, thyroid cancer, uterine cancer, urethral cancer, ureter cancer, vaginal cancer, vulval cancer, cancer of the peritoneum, hepatocellular cancer, gastric or stomach cancer (including gastrointestinal cancer), cervical cancer, ovarian cancer, liver cancer, bladder cancer, hepatoma, breast cancer, colon cancer, colorectal cancer, endometrial or uterine carcinoma, salivary gland carcinoma, kidney cancer (e.g., renal cell carcinoma), liver cancer, prostate cancer, vulval cancer, thyroid cancer, hepatic carcinoma, anal carcinoma, penile carcinoma, melanoma, and various types of head and neck cancer.

cAg(s) can be AW non-Hodgkin's lymphoma, e.g., B-cell lymphoma(s) or T-cell lymphoma(s), e.g., diffuse large B-cell lymphoma, mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, CNS lymphoma, precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral TC lymphoma).

In aspects, cAg(s) are associated with proteins (PPTs) expressed in/on cancer cells comprising cancer cell marker(s), e.g., CD123: CD2, CD19, CD20, CD30, CD38, CD40, CD52, CD70, EGFR/ERBB1, IGF1R, HER3/ERBB3, HER4/ERBB4, MUC1, TROP2, cMET, SLAMF7, PSCA, MICA, MICB, TRAILR1, TRAILR2, MAGE-A3, B7.1, B7.2, CTLA4, or PD1.

In aspects, the FP lacks the K9Melapoly PE. In aspects, the FP lacks any canine melanoma Ags, any canine TAA(s), or any melanoma Ags.

In aspects, cAg(s) are associated with solid tumor cancer cells. In aspects, cAg(s) are associated with non-solid-tumor cancer cells (e.g., a blood cancer, non-tumor-forming breast cancer, or liquid tumor cancer).

In aspects, cAgs comprise TCE(s). In aspects, TCE(s) comprise MHCIE(S), MHCIIE(s), or both. In aspects, TCE(s) comprise both MCHIE(s) and MHCIIE(s). In aspects, TCE(s) comprise ≥3 TCE(s). In aspects, OSMOA of the TCE AARS(s) in the CEP are at least 15 AAs, at least 18 AAs, or at least 20 AAs in length. In aspects, OSMOA shorter TCE(s) are associated with upstream or downstream flanking sequences. In aspects, SMOA TCE(s) are TH1 TE(s). In aspects, one or some (OOS) TCE(s) are T17 TE(s). In aspects, OSMOA cAg(s) comprise BCE(s). In aspects, CEPs lack DCA-associated BCE(s).

cAg(s) can comprise any suitable number of cancer Ag(s) classified into any one or more (OOM) suitable cancer Ag classification(s). In aspects, cAg(s) comprise AARS(s) of or related to PPT(s) over expressed in cancer cells, expressed in association with cancer-causing DCA(s), or both. In aspects, OSMOA cAg(s) in a CEP comprise tumor-associated antigen(s) (TAA(s)). In aspects, OSMOA cAg(s) comprise cancer testis Ag(s)-CTA(s)/cancer germline Ag(s)-CGA(s)) (e.g., MAGE-A1, NY-ESO-1, and SSX-2 Ag(s)). In aspects, OSMOA cAg(s) in a CEP comprise viral cancer-associated Ag(s) (VCAA(s)). In aspects, OSMOA cAg(s) in a CEP comprise tumor-specific Ag(s) (TSA(s) or Ag(s) classified as neoantigen(s) (NA(s)) (or Ag(s) classifiable as either). In aspects, OSMOA cAg(s) in a CEP are oncoprotein Ag(s). In aspects, cAg(s) comprise a mixture of two or more of such Ag types (e.g., 3 or more such Ag types). In aspects, cAg(s) comprise OOM TAA(s) and OOM TSA(s)/NA(s). In aspects, OSMOA TSA(s)/NA(s) in a CEP are shared (public) NA(s) (SNA(s)).

In aspects, OSMOA cAg(s) of a CEP are related to differentiation antigens (e.g., Gp100, Melan-A/Mart-1, and Tyrosinase). In aspects, OSMOA cAg(s) in a CEP are oncofetal Ag(s) (e.g., carcinoembryonic antigen (CEA)). In aspects, OSMOA cAg(s) are antiapoptotic proteins (e.g., livin and survivin), hTERT, or a tumor suppressor protein (e.g., p53). In aspects, OSMOA cAg(s) of a CEP are mimotopes of tumor-associated carbohydrate antigens (TACAs). In aspects, cAg(s) comprise CTs of these or other cAg(s) described elsewhere.

In aspects, OSMOA cAg(s) of a CEP are xenogenetic Ag(s), comprising sequences CPCGCOSCO or CO an AARS of or associated with homolog of OOM of the CAg(s) described here in a TR (e.g., a CAg comprising an AARS of a CAg from a NHA cancer that is a human homolog of a human cancer CEA, MAGE, or Tyrosinase). In aspects, OSMOA CAg(s) are hybrid/chimeric xenogenetic Ag FPs comprising both TR-endogenous CAg-related AARS(s) and xenogenetic (other species) AARS(s) from related homolog(s). In aspects, such a CAg(s) exhibits DOS IR(s) (e.g., MHCII Ag presentation) than the corresponding human or non-FP counterpart cAg (in the same context). The use of xenogenetic Ag(s) is not limited to cancer, however, and can be applied to any CEP described here (e.g., viral AgES CEPESCs).

In aspects, a CEP comprises synthetic epitope(s) in combination with CAg(s). In one exemplary aspect, the synthetic epitope is the pan DR epitope (PADRE). This aspect can be applied to other types of CEPESCs described in this disclosure (e.g., viral Ag CEP(s)).

In aspects, CAg(s) comprise a mix of immunodominant & unconventional epitopes. In aspects, such a mix is delivered by associated but separate administration of CEPESCs or CEPESC(s) and other AgES NAM compositions (e.g., a 2nd composition comprising a NGDICRTS-Ag FP, such as a DEC-205-binding domain/Ag FP). In aspects, most, generally all, substantially all, or all of the CAg(s) in a CEP exhibit a DOS in a significant number of TR(s). In aspects, such CEP(s) comprise a collection of CAg(s) selected for control of immunodominance, comprise CAgV(s) modified for reduced immunodominance, or both. In aspects, CAg(s) comprise unnatural immunity epitope(s).

In aspects, CAg CEPs comprise PE(s). In aspects, PE(s) comprise FL(s), MSL(s), MSFL(s), SCSs, or CT. In aspects, gDFPAg(s) comprise PE(s).

In aspects, OSMOA CAg(s) of a CEP are associated with ITS(s), e.g., PTPS(s), e.g., polyUb(s). In aspects, CAg(s) are AW non-PTPS ITS(s).

In aspects, OSMOA CAg(s) comprise antigen recognition/presentation facilitating sequences (ARPFS(s)). In aspects ARPFS(s) comprise HSP(s), MHC(s), or both. In aspects, CAg CEPs comprise OOM ES(s) coding for anti-cancer PPT(s) or AARS(s), such as anti-angiogenic factor(s), e.g., anti-VEGF PPT(s). In cases CAg CEP(s) comprise anti-cancer Ab PPTs (e.g., Ab FPs or other Ab PPTs). In cases CEPs lack Ab AARS(s).

CAg(s) are KNOWN. Relevant teachings concerning such antigens and other compositions, methods, techniques, and principles relevant to and can be adapted to combine/incorporate in these aspects of the invention are described in, e.g., Luo W et al. Cancer Cell Int. 2020; 20:66. Published 2020 Mar. 4; Curran M A et al. Annu Rev Med. 2019; 70:409-424; Maeng H M et al. F1000Res. 2019; 8: F1000 Faculty Rev-654. Published 2019 May 13; Pender A et al. Cancers (Base1). 2018; 11(1):1. Published 2018 Dec. 20; Lee S H et al. Hum Vaccin Immunother. 2015; 11(8):1889-1900; Buonaguro L, et al., Clinical and Vaccine Immunology Jan. 2011, 18 (1) 23-34; Lopes, A et al. J Exp Clin Cancer Res 38, 146 (2019); New Strategies to Improve Therapeutic Vaccines (Seyyed Shamsadin Athari Ed.) (2018) (e.g., the chapter by Yu, C. et al.); Pan R Y et al. J Immunol Res. 2018; 2018:4325874; Terbuch A et al. Vaccines (Base1). 2018; 6(3):52; Yamamoto, T. N., et al. Nat Med 25, 1488-1499 (2019); and Hollingsworth R E et al. NPJ Vaccines. 2019; 4:7.

Examples of CAg(s) adaptable to CEP(s) include Her2/Neu CAgs; abnormally expressed PPT CAgs (e.g., MAGE-A3, BAGE, AFP, XAGE-1B, mesothelin, PRAME, or Muc-1); melanoma-associated CAgs (e.g., Mart-1, GP-100, tyrosinase-related protein 1, tyrosinase-related protein 2, etc.); prostate cancer-associated CAgs (e.g., PSA, PAP, and PSMA); & B cell leukemia/lymphoma-associated CAgs (Ig gamma or Ig kappa). CAg(s) can fall within both these & other classifications (e.g., tyrosinase is a melanoma-associated CAg and survivin and NY-ESO are abnormally expressed PPTs).

In aspects, OSMOA CAg(s) of a CEP are viral cancer-associated antigen(s) (VCAA(s)). VCAAs typically arise from oncogenic viral PPTs, and, accordingly, also can be classified as oncoproteins (other oncoproteins are discussed below). Readers will recognize that there also can be overlap between viral pathogen Ag(s) and VCAA(s) (i.e., in one aspect a CEP comprises OOM Ag(s) that can be classified both as anti-viral Ag(s) and VCAA(s), as in the case of HPV Ag(s)). In aspects, a VCAAgES CEPESC induces DOS IR(s) against an oncogenic virus. In aspects, delivery of an EA of a VCAAgES CEPESC induces DOS reduction in viral neoplasia(s).

Well-studied VCAAs include the HPV E6/E7 PPTs. In aspects, CEP(s) comprise one or both type of Ag(s). In aspects, OSMOA CAg(s) in a CEP are HPV16 VCAA(s), HPV18 VCAA(s), or a combination (E6, E7, or other Ag(s)). In aspects, HPV AgES CEPESCs can be characterized in that (a) HPV Ag(s) in the CEP are associated with PTPS(s); gDSS, mGDS(s), AgV(s) (e.g., GSRV Ag(s), such as a GSRV EP7, e.g. SEQ ID NO:716 or a FF/FV); (b) the CEP comprises or is delivered in association with ITII(s) (e.g., hEAT-2 or a FF or FV); (c) are expressed from NAV(s) (e.g., mRNA(s) or CaPNP-associated plasmid(s)); are expressed from multiple NAM(s); (d) comprise HPV Ag PE(s) comprising MSL(s), FL(s), or MSFL(s), self-cleavage site(s), or combinations; or (e) comprise a combination of two or more of any aspect of (a)-(d). In aspects, HPV AgES CEPESC(s) comprise OOM aspects of at least three of (a)-(d). In aspects, HPV AgES CEPESCC(s) comprise OOM aspects of all four of (a)-(d). In aspects, an HPV Ag CEP comprises capsid protein L2 Ag(s). In aspects, OOM of such HPV AgES CEPESCs exhibit DOS improved IR(s) as compared to gDAgFP(s) comprising HPV Ag(s) described in the Wistar Art. In aspects, the improved IR comprises enhanced memory immunity effect(s). In aspects, delivering an EA of OOM of such HPV AgES CEPESC(s) results in DOS reduction in HPV associated cervical carcinomas; HPV-positive head and neck cancers; or both. In aspects, TR(s) treated with such HPV AgES CEPESC(s) have not been exposed to HPV. In aspects, TR(s) have been diagnosed with a premalignant HPV-associated disease such as cervical intraepithelial neoplasia (CIN) or vulvar intraepithelial neoplasia (VIN) and the delivery of an EA of the CEPESC DOS reduces progression of such conditions to cancer. In aspects, delivery of EA(s) HPV AgES CEPESC(s) induces DOS reductions in HPV-associated cervical intraepithelial neoplasia (CIN), cervical cancer, or head and neck cancer in HPV challenged TR(s) or HPV-infected TR(s).

In aspects, CEPs comprise Merkel cell carcinoma virus (MCCV) (Merkel Cell Polyoma virus) Ag(s). In an aspect, CAg(s) comprise MCCV large T Ag(s), small T Ag(s), or combinations. In aspects, EA(s) of such CEPESC(s) DOS induces CE(s) against or treats/prevents Merkel cell carcinoma(s) in TR(s). In aspects, CEPs comprise human T lymphotropic virus-1 (HTLV-1) Ag(s). In aspects, a CEP comprises HTLV-1 tax PPT Ag(s). In aspects, delivery of EA(s) of related CEPESC(s) induce CE(s) against or treats or prevents HTLV-1-associated neoplastic transformation, spastic paresis induction, or both.

In aspects, a CEP comprises EBV Ag(s). In aspects, a CEP comprises EBV EBNA-1, EBV LMP2, or a combination thereof. In aspects, delivery of an effective amount (EA) of such CEPESC(s) DOS treats or prevents EBV-associated nasopharyngeal carcinoma, Burkitt's lymphoma, or both. In aspects, a CEP comprises CVM Ag(s). In aspects, a CEP comprises polyomavirus Ag(s) (e.g., middle T (mT) oncoprotein Ag(s)). In aspects, CEPs comprise human herpesvirus 8 (HHV-8) (KSV) Ag(s). In aspects, delivery of EA(s) of an HHV-8 AgES CEPESC(s) prevents or treats Kaposi's sarcoma (KS) or an HHV-8-associated lymphoproliferative disorder (e.g., primary effusion lymphoma & multicentric Castleman's disease).

In aspects, an anti-cancer CEPESC comprises HBV Ag(s), HCV Ag(s), or both. In aspects, TR(s) receiving such CEPESCs are pre-cancerous or have not been exposed to the indicated DCA and the delivery of an EA of CEPESC(s) DOS reduces the inflammatory tissue environment in virus-infected TR(s) or TR(s) subsequently challenged with such viruses.

In aspects, CEPs comprise tumor associated antigen(s) (TAA(s)), other cancer antigen(s) (CAg(s)), or both. In aspects, OSMOA CAg(s) of a CEP are differentiation TAA(s) (e.g., PSA, mammaglobin-A, or tyrosinase). In aspects, OSMOA CAg(s) of a CEP are overexpression Cag(s) (e.g., HER2 and TERT). In aspects, OSMOA CAg(s) of a cEP are CGA(s) (e.g., MAGE or BAGE). In aspects, OSMOA CAg(s) of a CEP are oncofetal CAg(s) (e.g., CEA or TPBG). In aspects, CAg(s) of a CEP comprise 2, 3, or 4 of such Ag types.

Several TAAs are known and can be incorporated into CEPs (including examples provided above). Well-studied TAAs include CEA, MAGE Ags (e.g., MAGE-A Ags), and MUC Ags. In aspects, such Ag(s) are incorporated in CEP(s) or FFs or FVs thereof are incorporated into CEPs.

In aspects, a CEP comprises MAGE CAg(s) (e.g., MAGE-1, MAGE-3, MAGE-4, MAGE-12, or other MAGE Ag(s) (see e.g., WO99/40188).

In aspects, a CEP comprises prostate cancer CAg(s). Examples of such CAg(s) include prostate specific antigen (PSA), PAP, PSCA (PNAS 95(4) 1735-1740 1998), PSMA; Prostase (Prostase Ag(s) are described in, e.g., P. Nelson et al. Proc. Natl. Acad. Sci. USA (1999) 96, 3114-3119; Ferguson, et al. (Proc. Natl. Acad. Sci. USA 1999, 96, 3114-3119; WO 98/12302; WO 98/20117; and WO 00/04149); P501S (SEQ ID NO:113 of WO98/37814); PS108 (WO 98/50567); STEAP (PNAS 96 14523 14528 7-12 1999); and Ag(s) described in WO98/37418 and WO/004149. Additional prostate CAg(s) and related PMCs adaptable to aspects are described in Zahm C D et al. Pharmacol Ther. 2017; 174:27-42; Michael A, et al. Expert Rev Vaccines. 2013; 12(3):253-262; & Laccetti Curr Opin Urol. 2017; 27(6):566-571.

In other aspects, a CEP comprises AARS(s) of or related to Plu-1 Ag(s) (J Biol. Chem 274 (22) 15633-15645, 1999), HASH-1 Ag(s), HasH-2 Ag(s), Cripto Ag(s) (Salomon et al Bioessays 199, 21 61-70, U.S. Pat. No. 5,654,140) Criptin Ag(s) (e.g., U.S. Pat. No. 5,981,215), tyrosinase, and survivin. In aspects, CEP(s) comprise CAg(s) of or related to Muc-1, Muc-2, EPCAM, her 2/Neu, Wilms tumor-1 (WT-1/WT1), mammaglobin (U.S. Pat. No. 5,668,267) or those disclosed in WO/00 52165, WO99/33869, WO99/19479, WO 98/45328. Additional CAg(s) of which sequences or related sequences can be incorporated in CEPs include MART, trp, gp100, MUM⁻¹-B (melanoma ubiquitous mutated gene product), HER-2, Ras, PSA BCR-ABL, CASP, CDK, p53, TWI, PAP, telomerase, EGFR, LMP-1, PSMA, PSA, PSCA, tyrosinase, TRP, gp100, SSX-2, CD19, or CD20. ERG, Androgen receptor (AR) antigen, PAK6 antigen, a Prostate Stem Cell Antigen (PSCA), Stratum Corneum Chymotryptic Enzyme (SCCE) antigen, human telomerase reverse transcriptase (hTERT) antigen, a Proteinase 3 antigen, a Tyrosinase Related Protein 2 (TRP2) antigen, a High Molecular Weight Melanoma Associated Antigen (HMW-MAA), a synovial sarcoma antigen, a X (SSX)-2 antigen, an interleukin-13 Receptor alpha (IL13-R alpha) antigen, a Carbonic anhydrase IX (CAIX) antigen, a p97 melanoma antigen, a KLH antigen, a HSP-70 antigen, a beta-HCG antigen, or a Testisin antigen. In aspects, a CEP comprises PRAME, BAGE, Lage (also known as NY Eos 1) SAGE and HAGE (WO 99/53061) or GAGE (Robbins and Kawakami, 1996, Current Opinions in Immunology 8, pps 628-636; Van den Eynde et al., International Journal of Clinical & Laboratory Research (1997); Correale et al. (1997), Journal of the National Cancer Institute 89, p 293. These and other antigens that can be incorporated or adapted for use in CEP(s) are described in US20190290686, US20190032064, and US20180094071.

In aspects, CEP(s) comprise NY-ESO-1 Ag(s) & delivering EA(s) of CEPESC(s) induce DOS IR(s) regarding bladder cancer(s) or treats/prevents BC. In aspects, CEPs comprise HER2 Ag(s) & delivering EA(s) of such CEPESC(s) induce DOS IR(s) against breast cancer(s) or treats/prevents it. In aspects, a CEP comprises CEA Ag(s) and delivering EA(s) of the related CEPESC(s) induce DOS IR(s) against colorectal cancer(s) or treats it. In aspects, a CEP comprises WT1 Ag(s) and delivering EA(s) of the related CEPESC(s) induce DOS IR(s) against leukemia, or treats or prevents it. In aspects, a CEP comprises MART-1, gp100, or tyrosinase Ag(s), or combinations, and delivering EA(s) of the related CEPESC(s) induce DOS IR(s) against colorectal cancer(s) or treats or prevents such cancers.

In aspects, a CEP comprises up-regulated lung cancer (URLC10) epitope peptide Ag and an EA of the related CEPESC induces DOS IR(s) against non-small cell lung cancer(s). In aspects, a CEP comprises surviving Ag(s) and an EA of CEPESC induces anti-ovarian cancer IR(s) or treats or prevents ovarian cancer. In aspects, CEPs comprise MUC1 CAg(s) & delivering EA(s) of such CEPESC(s) induces DOS IR(s) against pancreatic cancer(s). In aspects, a CEP comprises a MUC2 CA(g) and delivering an EA of the related CEPESC induces DOS IR(s) against prostate cancer or treats or prevents the disease. In aspects such a CEP further comprises additional prostate cancer Cag(s), e.g., PSA Ag(s).

In aspects, CEP(s) with Ag(s) that PCGCOSCO or CO TAA Ag(s) are associated with 1 CI or 2+ CIs (e.g., a NGDCI, such as PD-L1 CI, CD112R CI, CRACC CI, or FAP CI, and a gDS CI), or three CI(s). In aspects, a primarily TAA CEP comprises ITII(s) (e.g., hEAT-2 or an FF or FV). In aspects, a primarily TAA CEP comprises 1 or 2 CIs and ITII(s). In aspects, OSMOA TAA Ag(s) of a CEP are AgV(s). In aspects, OSMOA TAA AgV(s) are FPs comprising 1+ AARSs that enhance recognition of TAA(s) (e.g., a HSP, viral Ag, bacterial Ag, or synthetic epitope). In aspects, TAA AgV(s) include AARS(s) of TAA homolog(s).

In aspects, CAg(s) of CEPs comprise mutational Ag(s) (a.k.a., mutanome-derived antigens). Mutational Ag(s) are mutated “self-antigens,” typically arising from non-synonymous mutations.

In aspects, OSMOA of the mutational CAg(s) in a CEP are classifiable as tumor specific antigens (TSAs). Typically, non-synonymous mutations generate CAg(s) that are more “specific” in being associated with certain cells, populations, and carcinogenesis processes. Importantly, TSAs are not expressed on normal cells and thus typically do not induce autoimmunity and typically are subject of less tolerance than TAAs (TAAs in contrast often are PPTs that are overexpressed in cancer cells, but also expressed in low levels in normal cells). TSAs are described in, e.g., Hollingsworth R E, et al. NPJ Vaccines. 2019; 4:7. In aspects, TSA(s) are related to PPTs involved in cancer development (e.g., oncogenes and tumor suppressor genes, such as Ras and Bcr-Abl (such CAg(s) are further discussed below). In aspects, an anti-cancer CEP which PCGCOSCO of CO TSA(s) exhibits DOS reduced autoimmunity effects, detectably reduced tolerance, or both than a comparable product PCGCOSCO TAA(s). The “specificity” of TSA(s) is a relative term, however, as such mutations can occur in several tumors/cancers and sometimes even in normal cells. In aspects, TSAs of a CEP occur in less than about 65%, ≤˜50%, or less than about 45% of a population.

In aspects, OSMOA of the CAg(s) in a CEP are classifiable as neoantigens. Neoantigens are a class of TSAs that similarly arise from mutations which are more common in cancer cells. Neoantigens are highly individual-specific and usually do not involve known oncogenes. Given their individual and highly specific nature neoantigens can often be DOS more effective in inducing anti-cancer IR(s) than TAAs. In an aspect, a neoantigen is present in less than about 2%, ≤˜1.5%, ≤˜1% of a population, or less than 0.5% of a population; are recognized by ≤˜1%, ≤˜0.5%, or ≤˜0.25% of the T cells in TR(s); or both (before treatment).

An intermediate class of TSAs adaptable to CEP(s) are “shared neoantigens” (SNAs) (sometimes also called “public neoantigens”) (See, e.g., Zhao W. et al. Pharmacogenomics. Published 19 May 2020). In an aspect, OSMOA of the CAg(s) in a CEP are classifiable as shared neoantigens. In aspects, shared neoantigens are present in between about 1-30%, 1.5-30%, 2-30%, 1-25%, 1.5-25%, 2-25%, 1-20%, 1.5-20%, or about 2-20% of a population; are recognized by ≤5%, ≤2.5%, or less than −1% of T-cells in TR(s) or both (before treatment).

Examples of known SNA epitopes include KQMNDARHG (SEQ ID NO:741) associated with breast cancer cells, LSKITEQEK (SEQ ID NO:742), and STRDPLSKI (SEQ ID NO:743). These and other SNA(s) are described in Wood, M. A et al. BMC Cancer 18, 414 (2018). Other examples of neoantigens/SNA(s) include BRAF V600E (Liu Q et al. Cancer Immunol. Immunother. 2018; 67:299-310) and KRAS G12D (Chaft J. E. Clin. Lung Cancer. 2014; 15:405-410). Additional neoantigens are the frameshift peptide (FSP) neoantigens available through Creative Biolabs (Shirley, NY, USA-creative-biolabs.com/vaccine). Muti-neoantigen PEs have been described by D′Alise, A. M et al. Nat Commun 10, 2688 (2019). Shared neoantigens are also described in Christopher A. J Exp Med 2 Jan. 2018; 215 (1): 5-7. Numerous additional tumor-specific neoepitopes are defined in WO2016187508 (see Tables 1-9). Any of these TSA(s) can be incorporated into CEP(s) individually, in combination, or in combination with other CAg(s).

In aspects, OSMOA TSA(s) of a CEP (e.g., SNA(s) or neoantigen(s) of a CEP) are associated with a “driver gene” PPT(s) (i.e., a PPT necessary for tumor survival). Examples of such genes associated with neoantigens/SNA(s) include PIK3CA, FAT4, BRCA2, GNAQ, LRP1B, and PREX2 (See, e.g., Zhou J et al. Biomed Res Int. 2019; 2019:8103142. In aspects, OSMOA of the CAg(s) of a CEP comprise such driver gene-associated neoantigens/SNA(s).

In aspects, OSMOA neoantigen(s)/SNA(s) of a CEP are already present in TR(s). In aspects, OSMOA neoantigen(s)/SNA(s) of a CEP are not present in TR(s). In aspects, a CEP comprises a mix of neoantigen(s)/SNA(s) that are present in a TR prior to delivery of the CEPESC and neoantigen(s)/SNA(s) not present in the individual prior to CEPESC delivery.

In aspects, CEPs comprise a mix of TAA(s) & TSA(s). In aspects, a CEP comprises ≥3 CAg(s) including, i.a., 1+ TAA(s) and TSA(s).

In aspects, CEP(s) comprise TSA(s) associated with mutations in a RAS protein (Kras), p53, or both. In aspects, such CEP(s) comprise microbial antigen(s), viral antigen(s), synthetic antigen(s), or combinations. In aspects, such CEP(s) include neoantigen(s)/SNA(s).

In aspects, CEP(s) comprise PCRA(s) or CRA(s) that are TSA(s), such as SNA(s). In aspects, the PCRA(s) is from a well characterized cancer, such as a cancer of hematological origin, such as a B cell lymphoma. In aspects, such a TSA comprises a mutated immunoglobulin idiotype (Ig Id) of a B cell receptor (BCR). Such an approach to identifying PCRA TSA(s) is described in Biernacki M et al. Frontiers in Immunology. 11:121 (2020).

In aspects, OSMOA of the TSA(s) of a CEP are from a carcinogen-induced cancer. In aspects, a TSA-ES CEPESC is used to treat such a cancer.

In aspects, a TSA is an AARS of or associated with PPT(s) expressed in melanoma, lung cancer and bladder cancer cells. In aspects, a TSA is an AARS of or associated with PPT(s) expressed in a tumor associated with DNA mismatch repair defects. In aspects, AARS(s) of or associated with PPT(s) of any such cell type are incorporated in CEP(s) as PCRA(s). In aspects, a CEP comprising any such PCRA(s) or CAg(s) comprises one, two, or more CI(s) (e.g., gDS CI(s); NGDCI(s), such as a PD-L1 CI, CTLA-4 CI, CRACC CI, FAP CI, or CD112R CI, e.g., an Ab, Ab FP, or multimeric trap; or both) and ICSTAP(s)/ICITM(s) (e.g., EAT-2(s), SAP(s), or combinations thereof (CT)).

Putative TSA PCRA(s) can be identified through sequencing screening (e.g., DNA or RNA sequencing) focused on finding genomic aberrations that are effectively expressed (e.g., comparing cancer and normal cells using high throughput sequencing, e.g., using whole exon screening); using in silico methods to predict which of the mutations will be presented to T-cells based on proteasome processing and binding affinity to MHC molecules (preferably of the relevant species/population/TR); and testing the identified PCRA(s) in appropriate constructs. Relevant methods for identifying PCRA that are putative TSA(S) and compositions/methods applicable/relevant to the aspects described here are described in Peng, M., et al. Mol Cancer 18, 128 (2019); Jiang, T., Shi, T., Zhang, H. et al. J Hematol Oncol 12, 93 (2019); and Schumacher T, et al. Annual Review of Immunology (2019) Vol. 37:173-200. TSA/neoantigen databases comprising potential PCRA(s) are described in Wu J, et al. Genomics Proteomics Bioinformatics. 2018; 16(4):276-282 and Xiaoxiu Tan et al. Database, Volume 2020, 2020.

In aspects, CEP(s) comprise oncoprotein(s). Oncoproteins are intended, without limitation, to refer to PPTs that are capable of inducing cell transformation. Oncogenes encoding oncoproteins arise via point mutations, gene amplifications & gene translocations in a normal gene (known as proto-oncogene) and result in altered gene expression or protein activity levels.

CEP(s) can comprise any suitable number of oncoprotein(s) of any suitable type. In aspects, OSMOA oncoprotein(s) of CEPs are apoptosis regulators. In aspects, OSMOA oncoprotein(s) of CEPs are cell cycle control proteins. In aspects, OSMOA oncoprotein(s) of a CEP are cell signaling proteins. In aspects, OSMOA oncoprotein(s) are DNA repair proteins. In aspects, OSMOA oncoprotein(s) are growth factors/mitogens (e.g., c-SIS). In aspects, OSMOA oncoprotein(s) are growth factor receptors. In aspects, OSMOA oncoprotein(s) are transcription factors (e.g., myc).

In cases OSMOA oncoprotein(s) are receptor tyrosine kinases (e.g., EGFR, PDGFR, VEGFR, etc.). In cases OSMOA oncoprotein(s) are cytoplasmic tyrosine kinases (e.g., Src-, Syk-ZAP-70-, or BTK-family tyrosine kinases). In cases, OSMOA oncoprotein(s) are GTPases (e.g., Ras).

In cases, OSMOA oncoprotein(s) of a CEP are associated with point mutation oncogenes (e.g., P53, RAS, CDC27, or P16). In cases OSMOA oncoprotein(s) of a CEP are associated with translocation oncogenes (e.g., BCR-ABL, TEL-AML-1, or PAX-3-FKHR). In cases, OSMOA oncoprotein(s) in a CEP are AW overexpressed oncogenes (e.g., P53 or Her-2).

In exemplary aspects, CEP(s) comprise AARS(s) AW PDGF, ERB-B, ERB-B2, K-RAS, N-RAS, C-MYC, N-MYC, L-MYC, BCL-2, BCL-1, MDM2, BCL-2, tetraspanin oncoprotein CD151, or H-Ras oncoproteins, or CT.

Oncoproteins also include, but are not limited to, viral proteins from RNA and/or DNA tumor viruses such as hepatitis B viruses, SV40 viruses, polyomaviruses, adenoviruses, herpes viruses, retroviruses and the like. Such viral CAg(s) are discussed above, but their inclusion here reflects that there is overlap in these categories, as in other categories of CAg(s).

In aspects, CEP(s) comprise Ag(s) against proto-oncogene EP(s). Proto-oncogenes include RAS, WNT, MYC, ERK, and TRK. In aspects, the proto-oncogene(s) are associated with a significant risk of cancer development. In an aspect, a proto-oncogene Ag is associated with MYC, which poses a risk of development of Burkitt's lymphoma.

In aspects, CEP(s) comprise cancer-related CRA(s), PCRA(s), or both. Aspects relating to cancer-associated PCRA(s) and CRA(s) are discussed above as well as here. A number of techniques known useful in the identification/selection of PCRA(s) are known in the art. An example of the application of experimental methods to the identification of a PCRA is described in Huang Y H et al. PLoS One. 2013; 8(6): e64365. Exemplary bioinformatics screening approaches are described in, e.g., Nezafat N et al. J Theor Biol. 2014; 349:121-134; Han K C, et al. Sci Rep. 2020; 10(1):5885; and Bachinsky M M et al. Cancer Immun. 2005; 5:6. Additional examples of CRA/PCRA methods in cancer contexts are exemplified below.

In aspects, OSMOA of the CAg(s) of a CEP are AgV(s) (CAgV(s)). CEPs can comprise any suitable type of AgV(s). In aspects, OSMOA of CAgV(s) are editopes. In aspects, editope(s) comprise AgV(s) with enhanced MHC binding, enhanced MHC:Ag:TCR binding, or both. In aspects, such AgV(s) comprise affinity-enhancing anchor residue modifications. In aspects, AgV(s) comprise mimotope(s). In aspects, AgV(s) comprise heteroclitic AARS(s). In aspects, AgV(s) comprise DIV(s), such as GSRV(s).

In aspects, CEPESC(s) are combined with CCC(s). Numerous additional types of combination compositions for cancer are described elsewhere here. Examples of CCC(s) include chemotherapeutics (e.g., chemotherapeutics that deplete immunosuppressive Tregs, MSDCs, or both, such as thalidomide derivatives), indoleamine 2,3-dioxygenase (IDO) inhibitors, STING agonists, agonists of selected TNF receptor family members (e.g., targeting CD40, 4-1BB/CD137, OX-40/CD134, or CD27), tumor suppressor proteins (e.g., APC, DPC4, NF-1, NF-2, MTS1, RB, and p53), inhibitors of undesirable cytokines (e.g., inhibitors of IL-20, TGF-β, IL-6, or combinations), agents that lower the tumor apoptosis threshold (e.g., cisplatin), and γC cytokines such as IL-7, IL-15, and IL-21 or IL-2. Peptidic agents in this list also can be expressed as “additional sequences” in the CEP. Additional agents include lipid and carbohydrate TAA(s) and immunoadjuvants (e.g., Sialyl Lewis, poly ICLC, or oxidized mannose (mannan)), CA19-9-targeted vaccines or CA19-9 Abs, or Anti-TGFβ. Method can comprise associated administration of such agents. Combinations also can comprise chimeric antigen receptor-(CAR-) T cell immunotherapy (e.g., anti-CD19 anti-CD20, anti-CD30, or anti-CD123 CAR-T), cellular therapies (e.g., ex vivo modified CTLs, DCs, and the like) (See, e.g., Todryk S, Jozwik A, de Havilland J, Hester J. Emerging Cellular Therapies: T Cells and Beyond. Cells. 2019; 8(3):284), toll-like receptor (TLR) agonists (e.g., flagellin, LPS, MPL (3-O-desacyl-4′-monophosphoryl lipid A), or CpG ODN), Prostaglandin E2, Arginase inhibitors, A2AR antagonists, metformin, adenosine receptor or ectonucleotidase inhibitors, DNA methyl transferase (DNMT) inhibitors, histone deacetylase (HDAC) inhibitors, HDAC inhibitors, DNMT-inhibitors, TAA-derivatives (e.g., keyhole limpet hemocyanin (KLH) carrier TAAs), allogeneic tumor cell vaccines, and autologous tumor vaccines. Additional compounds and principles are described in, e.g., Rossi J F et al. Cancer Commun (Lond). 2019; 39(1):34; Guo C, et al. Adv Cancer Res. 2013; 119:421-475; and Vermaelen K. et al. Front Immunol. 2019; 10:8.

In aspects, any above-described CAgES CEPESCs can be delivered in EA(s), 1+ times, to induce anti-cancer IR(s). In aspects, CAgES CEPESCs are delivered to induce protective/prophylactic IR(s). In aspects, CAgES CEPESCs are used therapeutically. In aspects, CEPESCs are delivered to TR(s) with a malignant cancer. In aspects, CEPESCs are delivered to TR(s) diagnosed with early-stage cancer (a cancer that is not invasive or metastatic or is classified as a Stage 0, I, or II cancer). In aspects, TR(s) is/are diagnosed with a precancerous condition (e.g., having growth that typically precedes or develops into a cancer). In aspects, TR(s) has/have a non-metastatic condition (e.g., a cancer that is benign or that remains at the primary site and has not penetrated into the lymphatic or blood vessel system or to tissues other than the primary site). In aspects, IR(s) comprise DOS reduction in tissue invasion.

In aspects, delivery of EA(s) of CAgES CEPESC(s) induces anti-cancer CE(s). In aspects, delivery of EA(s) of CAgES CEPESC(s) treats or prevents cancer(s). Cancers treatable by methods include any mentioned here or referenced in US20200325182. In aspects, CE(s) comprise reducing the size of an established tumor or lesion in the subject. In aspects, tumor(s) are reduced in size by about 50% to about 100%, about 60% to about 100%, about 70% to about 100%, or about 80% to about 100%. In aspects, CE(s) comprise DOS enhancement of tumor regression in TR(s). In aspects, CE(s) comprise increasing tumor regression by about 40% to about 100%, about 60%-100%, about 70%−˜100%, or ˜80% to ˜100%. In aspects, CE(s) comprise a DOS increase in the rate of survival of TR(s) with or that subsequently develop cancer(s). In aspects, CE(s) comprise a DOS increase in longevity. In aspects, CE(s) comprise a DOS enhancement in quality of life.

In aspects, CEPESC(s) are delivered by injection. In aspects, CEPESC(s) are delivered by mucosal administration. In aspects, CEPESC(s) are delivered through electroporation. In aspects, CEPESC(s) are delivered through biolistic (e.g., “gene gun”) methods.

In aspects, CAgES CEPESC(s) are administered 2+ times in anti-cancer methods. In aspects, different CEPESC(s) are administered to TR(s). In aspects, the same CEPESC is administered 2+ times in methods. In aspects, methods comprise administering CEPESC(s) and other agents (e.g., in co-administration or sequential administration). In an aspect, a CI or a CI-encoding NAM is administered several days (e.g., about 1 week) after an initial immunization with a CAgES CEPESC. In aspects, the second CI administration is a CAgES CEPESC comprising CI(s) (e.g., CI gDS(s), NGDCI(s), or both).

In aspects, IR(s) include a DOS reduction in aspect(s) of cancer progression (even(s) in the transformation of non-neoplastic cell(s) to cancerous, neoplastic cell(s), the migration of such neoplastic cells, the formation of tumors, or combinations, including cell crisis, immortalization and/or normal apoptotic failure, proliferation of immortalized and/or pre-neoplastic cells, transformation (i.e., changes which allow the immortalized cell to exhibit anchorage-independent, serum-independent and/or growth-factor independent, or contact inhibition-independent growth, or that are associated with cancer-indicative shape changes, aneuploidy, and focus formation), proliferation of transformed cells, development of metastatic potential, migration and metastasis (e.g., the disassociation of the cell from a location and relocation to another site), new colony formation, tumor formation, tumor growth, neotumorogenesis (formation of new tumors at a location distinguishable and not in contact with the source of the transformed cell(s)), and combinations. Aspects also include initiation, promotion, and progression, such as tumor initiation, tumor promotion, malignant conversion, and tumor progression (See, e.g., CANCER MEDICINE, 5th Edition (2000) B. C. Decker Inc., Hamilton, Ontario, Canada (Blast et al. eds.)). Tumor initiation aspects include induction of mitogenesis, compensatory cell proliferation, preneoplasia or hyperplasia, immortalization, and immunosuppression). In aspects, delivery of EA(s) of CAgES CEPESC(s) DOS reduces such event(s) in cell(s) in TR(s).

In aspects, IR(s) comprise increasing the number of CAg-specific IC(s). In aspects, such IC(s) comprise T cells. In aspects, IR(s) include DOS increases in IC(s) in the tumor microenvironment (TME). In aspects, IR(s) include DOS increases in CD4 cells, CD8 cells, or both, in the TME that are specific to CAg(s) in CEP(s). In aspects, IR(s) include DOS increases in anti-cancer cytotoxic IC activity in TR(s). In aspects, cytotoxic activity includes anti-cancer NKC activity, T-cell activity, or both.

In aspects, anti-cancer IR(s) lead to anti-cancer CE(s) in TR(s). In aspects, CE(s) comprise increase in average survival period, increase in TR(s) exhibiting DOS survival benefits, DOS reduction in disease symptom(s), DOS elimination of detectable disease cells, DOS induction of period(s) of disease-free or progression-free survival, objective durable cancer regression(s), or combinations. In aspects, CE(s) include increase in survival in TR(s) by at least about 3 months, ≥4 months, ≥6 months, ≥1 year, ≥18 months, ≥2 years, ≥2.5 years, ≥3 years, or ≥5 years, or longer.

In aspects, CAgES CEPESC(s) DOS outperform corresponding Ag peptide vaccines, corresponding viral vector vaccines comprising corresponding CAgES(s), or DNA vaccines comprising standard corresponding CAgES(s) with respect to IR(s) or CE(s). In aspect, CAgES CEPESC(s) DOS outperform current standard of care cancer treatments in TR(s) in terms of IR(s) or CE(s). In aspects, CAgES CEPESC(s) DOS outperform any of the corresponding CAgES compositions described in the Wistar Art, even when the same antigens are used (e.g., the gD-MELAPOLY or gD-E6/E7 constructs described therein).

In aspects, TR(s) are humans. In aspects, TR(s) are NHA(s). In aspects, TR(s) are companion animal(s). In aspects, NHA(s) are dogs.

In aspects, TR(s) are dogs of breeds at high risk for development of a cancer associated with CAg(s) in a CEP (See, e.g., Davis B W et al. ILAR J. 2014; 55(1):59-68)). E.g., EA(s) of compositions expressing EP(s) can include including CAg(s) that induce IR(s) to histiocytic sarcoma, can be delivered to flat-coated retrievers or Bernese Mountain dogs. In aspects, CE(s) comprise DOS reduction of cancer in joints, viscera, spleen, liver, lungs, muscle, etc.

In aspects, a CEPESC exhibits DOS CE(s) in both dogs and humans for cancer(s). In aspects, a CEPESC is evidenced through clinical studies to exhibit DOS CE(s) against cancer(s) in dog(s) and thereafter tested or used as a corresponding anti-cancer treatment in human patients.

In aspects, a CAgES CEPESC comprises CAg(s) associated with cancer(s) that are considered to exhibit similar/shared characteristics in two or more species. In aspects, such species comprise dogs and humans. In aspects, OSMOA CAg(s) are of or associated with cancer(s) in which some, most, or at least generally all similar molecular, physiological, or other characteristics, such as histopathology, tumor heterogeneity, gene expression patterns, immunology, metastasis, invasion, response to treatment, prognosis, or combinations are deemed similar in art or are similar by objective measurements (e.g., share a significant similarity as compared to comparisons with other cancer(s)) in two or more species, such as dogs and humans. Such conditions in dogs and humans include bladder cancer and lymphoma, which are described in detail to exemplify aspects.

In aspects, CAgES CEPESC(s) are delivered to humans, dogs, or other TR(s) at risk for or diagnosed with bladder cancer (BCCR).

In aspects, CAg(s) in anti-bladder cancer CEP(s) comprise AARS(s) of or related to Her2, NY-ESO-1, MAGE 1, MAGE 2, MAGE 3, MAGE4, LAGE-1, CT Antigen, MAGE C2, EGFR Receptor, Mucin1, Sialyl TN, or combinations. In aspects, OSMOA CAg(s) in anti-BCCR CEP(s) MUC-1, CT Ag, NY-ESO-1, or HER2 Ag(s), or combinations. In aspects, CAg(s) in anti-BCCR CEP(s) comprise PE(s) comprising 2, 3, 4, 5, or more CAg(s). In aspects, PE(s) comprise(s) 2, 3, or the specific TAA(s) cited in this paragraph. In aspects, anti-BCCR CEP(s) comprise oncoproteins relating to EGF-EGFR, ERBB2, UPK3A, ERBB2, FOXA1, ZEB1, ZEB2, CDH1, VIM, S100A1, S100A9, EGFR, or PPARG oncogenes, or combinations. In aspects, OSMOA CAg(s) in anti-BCCR CEP(s) are SNA(s). In aspects, CAg(s) comprise SNA(s) & TAA(s).

In aspects, most or all CAg(s) in CEP(s) are contained in gDFPAg(s). In aspects, OSMOA CAg(s) in PE(s) are associated with MSL(s), FL(s), MSFL(s), or self-cleavage site(s). In aspects, OSMOA CAg(s) are associated with PTPS(s). In aspects, OSMOA of such CAg(s) are AgV(s), e.g., GSRV AgV(s), Ag FPs (e.g., HSP—Ag FP(s)), editopes, or combinations.

In aspects, anti-BCCR CEP(s) comprise ITII(s) (e.g., EAT-2 PPT(s) or AARS(s), such as hEAT-2, mEAT-2, FFs of either, or FVs of any).

In aspects, anti-BCCR CEP(s) comprise CI(s). In aspects, anti-BCCR CEP(s) comprise at least 2 CI(s). In aspects, anti-BCCR CEP(s) comprise gDS CI(s), NGDCI(s), or both. In aspects, CEP(s) comprise or are administered in association with NGDCI(s). In aspects, NGDCI(s) include PD-1 CI(s), PD-L1 (a.k.a., PDL-1) CI(s), CTLA-4 CI(s), LAG3 CI(s), TIM CI(s), CRACC CI(s) (e.g., a CRACC-fc PPT), BLTA-4 CI(s), VISTA CI(s), or combinations. In aspects, anti-BCCR CEP(s) comprise Fibroblast Activation Protein (FAP) PPTs that DOS kill cancer-associated fibroblasts, reduces cancer-associated immunosuppression, or both (FAP can be administered in any CEP of any other aspect as well) (See, e.g., O'Connell, et al. Cancer Immunol Res Feb. 1, 2019 (7) A096).

In aspects, anti-cancer CEP(s), such as anti-BCCR CEP(s) or anti-lymphoma CEP(s) are delivered to TR(s) having a T cell inflamed tumor microenvironment (TME). In aspects, delivery of a CEP to a T cell inflamed TME DOS enhances activity or population of tumor-infiltrating lymphocytes (TIL(s)). In aspects, anti-cancer CEP(s) are delivered to TR(S) having a non-inflamed TME. The TME is understood in the art and generally refers to the matrix of cells surrounding and infiltrating tumors. In detected cancer, TMEs typically are immunosuppressive and both protect tumors from immune attack and nourish growth/progression of neoplastic cells and related biological signals and processes. In aspects, IR(s) induced by CEPESC(s) comprise DOS increases in IFNg production, DOS CD8-mediated cytotoxicity (e.g., in the TME), increased CD4 numbers/activity, or combinations. In aspects, IR(s) comprise detectable destruction of TME(s). In aspects, CE(s) comprise DOS tumor eradication, tumor growth cessation, tumor size reduction, delayed cancer progression, etc.

In aspects, a BCCR or another cancer comprises transitional cell carcinoma (TCC) cell(s). In aspects, CEPESCs are used to induce IR(s) in TCC cell(s) or in TR(s) diagnosed with TCC cell-associated cancer(s). TCCs are a major characteristic of BCCR(s) and can be used as an alternative characterization for cancers that typically include BCCR(s). In aspects, a BCCR TR is diagnosed as having C2-Squamous-like, C1-LUAD-enriched, or both C2 and C1 immunological cancer(s) groups.

In aspects, delivery of an anti-cancer CEPESC results in reduced activity or numbers of myeloid-derived-suppressor cells (MSDCs). In aspects, a CEP comprises a factor that reduces or blocks the activity of MSDCs (e.g., an ISNS, such as a CpG sequence) or is administered in association with such a factor (e.g., metformin). In aspects, an anti-cancer CEP comprises PPTs that downregulate/block TReg activity, such as anti-CTLA-4 PPTs, OX40 PPTs, and cytokines, such as IL-2. In aspects, CEP(s) comprise β-Catenin-blocking PPTs (e.g., PPTS comprising AARS(s) of β-Catenin blocking peptides (See, e.g., Hsieh, T et al. Sci Rep 6, 19156 (2016) or anti-β-Catenin Ab PPTs) or AAW β-Catenin blocking agent(s).

In aspects, IR(s) comprise DOS recruitment, expansion, activity, or combinations thereof of DCs, T-cells, BCs, NKCs, or combinations to tumors/TMEs. In aspects, such enhancements are in tissues/organs associated with improved cancer CE(s). E.g., in aspects CE(s) comprise DOS increases in cytotoxic T cells in peritumoral stroma.

In aspects, anti-BCCR CEPESC(s) are delivered AAW CCC(s), CCEPM(s), or both; or methods comprise combinations thereof. In aspects, CCEMP(s) comprise surgical excision of tumor(s), radiation therapy, or both. In aspects, CCC(s) comprise anti-cancer NSAID(s), such as piroxicam; chemotherapeutic agents (e.g., mitoxantrone, vinblastine, or both); or combinations. In aspects, CEPESCs combined with anti-cancer CCC(s), CCEPM(s), or both, DOS enhance anti-cancer IR(s).

In aspects, anti-BCRR CEPESC(s) DOS induce anti-BCCR IR(s). In aspects, EA(s) of anti-BCRR CEPESCs treat or prevent BCCR(s). In aspects, anti-cancer CEPESCs delivered to TR(s) diagnosed with cancer prior to treatment exhibit DOS enhanced periods between recurrence of cancer(s). In aspects, delivering anti-BCRR CEPESC(s) provide a remission rate of more than 35%, such as at least about 40%, ≥45%, ≥50% or more in TR(s). In aspects, delivering EA(s) of anti-BCRR CEPESC(s) result in disease stabilization in ≥50%, 60%, 65%, or 75% of TR(s). In aspects, average survival time in BCCR TR(s) receiving such methods are at least 1 year, 1.2 years, 1.5 years, 2 years, 2.5 years, or 3 years. In aspects, such methods DOS reduce cancer spread. In aspects, such methods DOS improve symptoms of BCRR(s), such as BCRR-associated urinary obstruction.

In aspects, anti-BCCR CEPESC(s) are delivered to humans, NHA(s), or both. In aspects, a NHA is a companion animal, such as a dog. In aspects, dog(s) comprise a terrier breed dog, collie bred dog, or beagle breed dog. In aspects, dog(s) comprise Scottish terriers, West Highland White Terriers, Eskimo Dogs, Shetland Sheepdogs, Keeshonds, Samoyeds, or Beagles. In aspects, CEP(s) comprise BCCR-associated PCRA(s). In aspects, PCRA(s) are screened to develop CRA(s) in dog(s) & such CRA(s) are delivered as PCRA(s) in CEP(s) in humans until CRA(s) are identified & a CEPESC that induces DOS protective/therapeutic CE(s) regarding BCRR in humans achieved.

In aspects, BCCR related CAg(s) comprise a MPHOSPH1-278 peptide (e.g., SEQ ID NO:717), a DEPDC1-294 peptide (e.g., SEQ ID NO:718) or CT. Such CAg(s) are described in Obara W et al. Jpn J Clin Oncol. 2012; 42(7):591-600. In aspects, BCCR CAg(s) comprise Tumor-Associated Glycoprotein 72 (See, e.g., Nagaya et al. Oncotarget. 2018; 9(27):19026-19038. In aspects, CCCs or AACs comprise BCG (Bacillus Calmette-Guerin) (See, e.g., Obara et al. Cancer Immunol Immunother. 2018; 67:1371-80). In aspects, CCCs/AACs comprise Mycobacterium bovis BCG or Mycobacterium brumae BCG-like compositions (See, e.g., Noguera-Ortega E et al. Sci Rep. 2018; 8(1):15102). In aspects, CAg(s) used in BCCR directed CEPESC(s) comprise 1+, 2+, or 3+ of NY-ESO-1, LAGE-1, MAGE-A1, MAGE-A3, MAGE-A4, MAGE-A10, CT7, CT10, and GAGE. In aspects, CAg(s) comprise MAGE-A3+Ag(s) (e.g., an Ag RVRHRSIOI to SEQ ID NO:455), NY-ESO-1 (e.g., a CAg RVRHRSIOI to SEQ ID NO:456), or CT, optionally in combination with 1 or both of MPHOSPH1 (M phase phosphoprotein-1) and DEPDC1 (DEP domain containing-1 protein) (see also U.S. Pat. No. 9,545,437). In aspects, CAg(s) comprise a HER2/neu CAg (e.g., a CAg RVRHRSIOI to SEQ ID NO:457). In aspects, such CEP(s) comprise anti-cancer NCMIMP(s), such as GM-CSF PPTs. In aspects, CAg(s) in such CEPESCs comprise BRAF CAg(s) (See, e.g., Cintolo J A et al. Melanoma Res. 2016; 26(1):1-11 & Liu Q et al. Cancer Immunol Immunother. 2018; 67(2):299-310). Examples of BRAF CAg(s) that can be included in CEP(s) include sequences RVRHRSIOI to OSMOA of SEQ ID NOs:451-454. BCCR-related aspects of the invention are further exemplified below.

In other aspects, CAgES CEPESC(s) are delivered to dogs, humans, or both, at risk for, or diagnosed with, lymphoma(s). In aspects, lymphoma(s) include(s) diffuse large B-cell lymphomas (DLBCL), marginal zone lymphomas (MZL), etc. In aspects, lymphoma comprises peripheral T-cell lymphoma-not otherwise specified (PTCL-NOS), T-zone lymphoma (TZL), or both. In aspects, lymphoma(s) comprise(s) B-cell malignancies, T-cell malignancies, etc.

In aspects a lymphoma to be treated/prevented is associated with a viral infection (e.g., HCV, HTLV, KSHV, HIV, or EBV infection). In aspects, an anti-lymphoma CAgES CEPESC comprises VACA(s) or other anti-viral Ag(s) in combination with other CAg(s) (e.g., TSA(s), TAA(s), or both). In aspects, lymphomas are virus-free lymphoma(s) are lymphomas not associated with a particular virus (e.g., EBV-free lymphoma(s)).

In aspects, CAg(s) in CEP(s) delivered to treat or prevent lymphoma(s) comprise AARS(s) of or related to lymphoma-associated Id PPT(s) (Ig-Id protein), survivin, MAGE-A4, Synovial sarcoma X (SSX2), PRAME, NY-ESO-1, TCL1 (Weng J, et al. Blood. 2012; 120(8):1613-1623), PASD1 (e.g., Cooper, C et al. Leukemia 20, 2172-2174 (2006), cTAGE (e.g., Usener D et al. J Invest Dermatol. 2003; 121(1):198-206), etc. In aspects, CAg(s) in CEP(s) against lymphoma(s) comprise lymphoma-associated oncogene AARS(s), such as AARS(s) of or AW CD95, TP53, PTEN, CD79B/A, IxBU, CARD11, API2-MALT1, EZH2, Jak2, or REL. In aspects, an anti-lymphoma CEP comprises TERT, MAGE1, NY-ESO1, or SSX-1 Ag(s), etc. In aspects, anti-lymphoma CEPs comprise 2, 3, 4, or more CAg(s).

In aspects, CEP(s) delivered for treating lymphoma(s) comprise or are administered in association with CI(s) (e.g., PD-L1 CI(s), PD-1 CI(s), CTLA-4 CI(s), FAP CI(s), CRACC CI(s) (e.g., CRACCFc), CDR112 CI(s), Lag-3 CI(s), etc.). In cases, CEP(s) for lymphoma(s) comprise or are delivered in association with NANCIPI(s), e.g., GMCSF or other cytokines. In aspects, CEP(s) comprise or are delivered AW with a bruton tyrosine kinase inhibitor, PI3K inhibitor or both. In aspects, CEP(s) comprise or are associated with anti-lymphoma Ab PPT(s), e.g., anti-CD20 Ab PPT(s). In aspects, anti-lymphoma CAgES CEPESC(s) comprise CCC(s) or are delivered in association with possible CCC(s) or CIIM(s), e.g., delivering a chemotherapeutic (e.g., CHOP (cyclophosphamide, doxorubicin, vincristine and prednisone), PCI-32765, KPT-335, or GS-9219), bone marrow transplant, applying radiation therapy, a thrombospondin-I PPT or mimetic, a NEMO-binding domain PPT that inhibits NFxB, a BTK inhibitor, or delivering any other anti-cancer CCC(s) (PMCs provided in, e.g., Zappasodi et al Front Immunol. 2015; 6:448 and Richards et al. Immunol Rev. 2015; 263(1):173-191). In cases, 6-month, 9-month, 12-month, 18-month, 24-month, 30-month, 3-year, 4-year, or 5-year survival in TR(s) after application of method(s) is ≥20%, 25%, 30%, 40%, or ≥˜50%. In aspects, method(s) achieve complete DOS remission for ≥50%, ≥65%, or ≥75% of TR(s) lasting a median ≥10 months, ≥12 mos., 18 mos., or ≥24 months. In aspects, TR(s) is/are diagnosed with significant risk of developing lymphoma based on indicator(s) (e.g., paraneoplastic hypercalcemia).

In aspects, the TR receiving anti-lymphoma CEPESC administration is a NHA, e.g., a companion animal, e.g., a dog. In aspects, dog(s) primarily comprising, generally comprised of, or comprised of (PCGCOOCO) breed(s) at high risk of lymphoma development, e.g., an Old English sheepdog, boxer, pointer, golden retriever, Rottweiler, St Bernard, Scottish terrier, bulldog, Irish wolfhound, Siberian husky, shih tzu, Airedale terrier, Cavalier King Charles spaniel, Yorkshire terrier, cocker spaniel, or basset hound. In aspects, such TR(s) receive PCRA(s) related to lymphoma. In aspects, CRA(s) identified in such animals are tested as PCRA(s) in human trials to develop CEP(s) comprising CRA(s) for treatment/prevention of lymphoma in humans.

### 2. gD AARS(s) and PPT(s)

CEP(s) comprise gD PPT(s) (gDP(s)). gDP(s) can include any suitable gD AARS(s). In aspects, CEPs comprise gDP(s) that are “gD proteins,” lacking any heterologous sequence(s). In aspects, CEPs include gDP(s) that are gDS fusion proteins (or gDFP(s)), such as gDAgFP(s). In aspects, CEP(s) comprise 2+ types of gDP(s) (e.g., 2+ gDAgFP(s)). In aspects, methods include delivery of 2+ types of gDPES CEPESCs 2+ times. In aspects, gDP(s) are multimeric PPTs, comprising 2+ associated gDP chains. In aspects gDP(s) are monomeric. In aspects, CEPs initially comprise immature forms of gDPs that are subsequently processed in one or more ways to form more mature PPTs. E.g., a CEP comprising 2+ gDP portions divided by SCSs can be cleaved forming two separate and more mature gDP portions. In aspects, gDP(s) can comprise a gDSS that is cleaved to form a more mature gDP. In aspects, gDPs can be post-translationally modified by, e.g., glycosylation in COEs. gDPs can include elements that result in 2+ of such modifications.

Typically, each discrete gDS in a gDP is ≥˜10 AARs or ≥˜15 AARs in length (e.g., ˜15-425 AAs). In aspects, OSMOA gDS(s) in a gDP are at least 20 AAs in length (e.g., about 20-400, 30-400, 20-380, 30-390, 25-375, 40-380, 40-360, 20-360, 25-350, 30-350, 50-350, or 50-400 AAs). In aspects, OSMOA gDS(s) of gDP(s) are at least about 15% smaller than WT gDP(s), such as 25% smaller, 33% smaller, 40% smaller, or 50% smaller. In cases, OSMOA gD(s) of gDP(s) are less than 200 AAs in length (e.g., 20-180 AAs, 20-120 AAs, or 20-100 AAs), less than 120 AAs in length, or less than 60 AAs in length (e.g., 15-45 AAs, 20-50 AAs, 15-30 AAs, 20-30 AAs, or 15-40 AAs). gDP(s) can comprise any suitable number of gDS(s). In aspects, gDP(s) comprise only 1 gDS. In aspects, gDP(s) comprise 2+ gDS(s). In cases, gDP(s) comprise 3, 4, or more discrete gDS(s). In aspects, gDP(s) comprise copies of gDS(s). For example, in an aspect, a gDP can be considered a fusion protein comprising 2 immature gDP(s) (e.g., two fused gDAgFP(s)) and a linker comprising a self-cleavage site, where such 2 fused gDP(s) can comprise 2+ identical gDS(s), different gDS(s), or combinations. Discrete gDS(s) can be organized in any suitable manner in gDP(s). Discrete gDS(s) can be directly linked (e.g., a gDSS and a receptor-binding gDS) or indirectly linked (e.g., separated by linker(s)). In gDSFPs, such as gDAgFPs, gDS(s) can be separated by Ag(s) or other FP components.

In gDSFPs, e.g., gDAgFPs, gDS can be CB on, ia, organization of gD and non-gD (heterologous-to-gD) AARSs. In general, gDSFPs can comprise any suitable number of non-gDS(s) and gDS(s) in any suitable relationship.

In aspects, a gDSS is positioned at the N-terminus of an immature gDP, such as a gDSFP. In aspects comprising gD receptor binding domain(s) (gDRBD(s)), gDRBD(s) are positioned at or near the N-terminus of the mature gDSFP (e.g., in the first 50%, 25%, 25%, or 7% of the AARS).

In aspects, non-GD sequences (NGDS(s), e.g., Ag(s)) are positioned directly or indirectly between two gDSs (in indirect positioning NGDS(s) can be bound to gDS(s) through intervening sequences, such as MSL(s) or FL(s) or self-cleavage site(s)). Such positioning is exemplified by the constructs in the Wistar Art subjected to actual in vivo experiments.

In aspects, NGDS(s) are positioned upstream, downstream, or both of any gDS(s) in a gDSFP. In aspects, OSMOA NGDS(s) of a gDSFP are positioned downstream of any gDS(s) in the PPT. In aspects, NGDS(s) are positioned both between gDS(s) and downstream of any gDS(s). In aspects, NGDS(s) are only located downstream of gDS(s). E.g., in gDAgFPs such PPTs can have a structure such as first gD sequence (gD1), first Ag sequence (Ag1), second gD sequence (gD2), and second Ag sequence (Ag2) (gD1-Ag1-gD2-Ag2). In aspects, a gDAgFP has a gD1-Ag1 structure, gD1-gD2-Ag1 structure, etc. In aspects, gDSFPs incorporate a gDSS, multiple Ag(s), ITS(s) (e.g., PTPS(s)), linkers (Ls), self-cleavage sites (SCSs), and the like, providing gDSFP structures such as gDSS-gD1-PTPS1-Ag1-PTPS2-L-SCS-L-PTPS3-Ag2-PTPS4-L-SCS-L-PTPS5-Ag3-PTPS6-L-SCS-L-PTPS7-Ag4-PTPS8-L-SC-LgD2-PTPS9-Ag9-PTPS10-L-SC-L-PTPS11-Ag10-PTPS12 or similar structures in which one or more Ls, SCSs, PTPSs, or Ags are removed or added to the structure.

In aspects gDAgFP(s) compre PE(s) & ITS(s). ITS(s) can be associated with PE sequence(s), each Ag of the PE sequence, or both. E.g., a gDAgFP can have the structure gD1-PTPS1-Ag1-L-Ag2-L-Ag3-L-Ag4-L-PTPS2-gD2, the structure gD1-PTPS1-Ag1-L-Ag2-L-Ag3-L-Ag4-L-PTPS2, or the structure gDSS-gD1-Ag1-Ag2-Ag3-Ag4-PTPS. In aspects, NGDS(s) are positioned upstream of gDS(s) (e.g., in aspects in which a gDSFP comprises a gD profusion domain (gDPFD)). Such gDSFPs can have a structure such as gDSS-NGDICRTS-Ag1-L-Ag2-L-Ag3-L-Ag4-L-Ag5-L-gDPFD or NGDSS-NGDICRTS-Ag1-Ag2-Ag3-gDPFD. In aspects, CEP(s) comprise 2+ gDSFP(s) having different arrangements of gDS(s) and NGDS(s).

In aspects, OSMGAOA gDS(s) in gDP(s) of CEPs are either of or RVRHROSI, SVSHSOCE, or both to gDS(s) of an uHV that infects a species that is the same as a species associated with OSMGAOA of the Ag(s) in the CEP. E.g., in one aspect CEPs comprise gDP(s) that comprise WT gDS(s) of HSV-1, HSV-2, or both, or FV(s) of such sequences and Ag(s) of a human cancer or Ag(s) of a pathogen DCA that infects humans. In aspects, OSMGAOA gDP(s) in CEPs are of or are related, very related, highly related, or substantially identical (RVRHROSI)/similar, very similar, highly similar, or compositionally equivalent (SVSHSOCE) to a gD that infects a first species that is different from the species that SMGAOA Ag(s) are from/related. E.g., in aspects, a CEP comprises a gDAgFP comprising HSV gDS(s) and mostly, generally, or only porcine or canine Ags or Ags of a DCA (e.g., a virus) that infects pigs or dogs.

In aspects, gDS(S) are identical to at least part of an AARS of a WT gD PPT. In aspects, a gDS comprises sequence(s) identical to functional domain(s) of WT gD(s). In aspects, gDS(s) comprise FFs of gDS(s). In aspects, gDS(s) comprise FVs of either a FL WT gD or a FF. In aspects, gDPs comprise gDS(s) that are a mixture of such different types of sequences.

In aspects, OSMGAOA of gDS(s) of gDP(s) in CEPs are AARSs in a WT HSV gD. In aspects, OSMGAOA gDS(s) of gDP(s) are AARSs that occur in a WT HSV-1 gD or HSV-2 gD (which can include any homologs/natural variants) (see, e.g., Uniprot Q69091 and P03172). In aspects, OSMGAOA gDS(s) of gDP(s) in CEPs are WT AARSs of non-HSV gDs. Examples of such non-HSV gDs include PRV/Suid alphaherpesvirus 1 gD (GenBank: AA062939.1); Gallid alphaherpesvirus-2/MDV-1 gD (Uniprot Q6764); EHV-1 gD (Uniprot Q6DLD9); EHV-4 gD (Uniprot AOAOYOA4Z5); Canid alphaherpesvirus 1 gD (Uniprot 041524); BHV-1 gD (Uniprot POCK29); BHV-5 gD (Uniptor Q65535); Simian herpes B virus gD (Uniprot A0A1X9WGB3); Meleagrid alphaherpesvirus 1 (MeHV-1) gD (Uniprot Q9DPP3); feline herpesvirus 1 (FeHV-1) (Feline viral rhinotracheitis virus) gD (Uniprot Q89634); and Gallid alphaherpesvirus 1 (Infectious laryngotracheitis virus) gD (GenBank Q67644). In aspects, OSMGAOA of gDS(s) in gDP(s) of a CEP are FL WT gDS(s). In aspects, OSMGAOA of gDS(s) in gDP(s) of a CEP are FFs of FL WT gDS(s).

In aspects, OSMGAOA gDS(s) in gDP(s) include WT gDS(s) of 1, 2, or more WT gDs. WT gDs herein can include any naturally occurring variations in WT gDs (e.g., G→S at AA50, 30A→V, 281L→I, 353A→V, 367 R→H, 369R→H, 371 R→Q of HSV-1 gD (see Uniprot Entry Q69091 and cf. to SEQ ID NO:29). Variant gDS(s) can include natural variations or non-naturally occurring variations. In aspects, OSMGAOA gDS(s) of a gDP are from 1 species of aHV, such as PRV, HSV-1, HSV-2, BHV-1, BHV-5, EHV-1, or EHV-4. Suitability of gDP(s) can be determined on, i.a., intended function. For example, with respect to HSV-1 gD proteins and polypeptides that can bind HVEM, substitution with a WT HSV-2 gD or N-terminal portion thereof typically would not be suitable, since such gDS(s) do not DOS bind HVEM.

In aspects, gDP(s) comprise chimeric gDS(s) comprising portions of gDS(s) of 2+gD homologs, such as HSV-1 gD and HSV-2 gD, PRV gD and HSV-1 gD, or Canid alphaherpesvirus 1 (CaHV-1) gD and feline herpesvirus-1 (FHV-1) gD. Suitable combinations of sch gDS(s) can function without exhibiting related or similar sequence composition (although such chimeric gDS will typically exhibit structural similarity to one or both counterpart gDS(s) (structural similarity is described elsewhere). In one example of such an aspects, a construct encodes an N-terminal portion of HSV-2 that replaces SMGAOA known/expected HVEM-binding domain of HSV-1 (e.g., residues 26-57 or 26-58 of HSV-1) combined with a C-terminal portion comprising, e.g., one of SEQ ID NOs: 56, 57, 58, 71, 72, 73, or 74. A chimeric gDS can comprise, e.g., a nectin-1 RBD that is a chimera of a HSV-1/HSV-2 gD RBD & a PRV RBD.

In aspects, gDP(s) in CEPs comprise 1+ variants of WT gDS(s) (gDV(s)). gDVS(s) are RVRHROSI, SVSHSSOCE, or both to 1+WT gDS. In aspects, a gDVS is RVRHROSI to an HSV gD (e.g., HSV-1 gD). In aspects, a gDVS is RVRHROSI to HSV-2. In aspects, gdVS(s) are RVRHROSI, SVSHSOCE, or both to at least one WT non-HSV gD homolog. In aspects, a gDVS is RVRHROSI to PRV gD. In aspects, a gDP comprises 2+ gDVS that are respectively RVRHR or SI to sequences of 2+WT gDPs.

In aspects, gDP(s) of CEPs DOS bind to Nectin-1, a Nectin-1 homolog, or both. In aspects, gDP(s) comprise RBDs of a WT gDP that bind Nectin-1/Nectin-1 homolog, a chimera of 2+ such gDPs, an FF of such WT gDD(s), or a FV of such gDS(s)/gDD(s). FFs of such gDS(s) typically are RVRHROSI, SVSHSOCE, or both, to Nectin-1-binding or homolog-binding WT gDS(s). In aspects, variant gDS(s) that serve as Nectin-1/homolog RBD(s) in gDP(s) exhibit DOS higher affinity for Nectin-1, a Nectin-1 homolog, or both, than the counterpart WT gDS. Examples of such gDS(s) are in PRV, HSV-2, and BHV-1, although BHV-1 gD Nectin-1 RBDs exhibit low affinity and FVs of BHV-1 gD Nectin-1 RBDs exhibit enhanced affinity (Connolly 2001, supra).

While gDV(s) can exhibit identity, similarity, or both to any suitable 1+WT gDS(s) (e.g., those described elsewhere), characterization of many aspects of gDS(s) and gDP(s) in this disclosure will primarily be exemplified in reference to HSV-1 gD. This practice is used herein for exemplification and conciseness only and any such disclosure will be understood to provide support, simultaneously and implicitly, for corresponding gDS(s)/gDP(s) of or that are related to HSV-1 gD homologs, such as those described elsewhere. In some parts of this disclosure explicit reference will be made to gD homologs to reinforce that the invention is not limited to HSV-1 gD-related gDSs.

gDP domains can be defined by function (e.g., RBDs), structure (e.g., immunoglobulin folds), or both (e.g., gDSSs). Functional & structural domains can overlap, as can different functional domains. Identifiable domains in HSV-1 gD, e.g., include: (1) in immature gDPs (igDPs) a gDSS (e.g., AAs 1-25); (2) an ectodomain, which typically is made up of (a) an N-terminal flexible region/domain (AAs ˜26-58), which can comprise all or parts of RBD(s) (discussed below); (b) a core domain (AAs ˜59-280) comprising an Ig-like V fold subdomain (AAs ˜80-210) and N-terminal and C-terminal flanking regions (AAs ˜59-79 and ˜211-280), which also can comprise part/all of RBD(s); (c) a flexible C-terminus domain (FCTD) (AAs ˜281-340), most of which is made up by a functionally-defined profusion domain (AAs ˜285-335), comprising 2 subdomains (PFD1, PFD2-AAs 285-310 and ˜310-335, respectively), and can comprise a N-terminal flexible region (NTFR) “shielding domain” (NTFRSD) when the gDP is in an unbound (receptor-free) state (AAs ˜313-331); and (d) a functional Glycosylphosphotidylinositol (GPI) anchoring domain (AAs 234-337) comprising a secretion promoting subdomain (234-294), overlapping parts of both the core domain & FCTD; (3) a transmembrane domain (TMD) (AAs ˜340-361); and (4) an intravirion/cytoplasmic domain (AAs ˜362-394) comprising an Arg/Lys rich anchor subdomain (AAs ˜365-381).

The use of the modifier˜in connection with the preceding description of WT gDDs arises from variations in gDPs & various characterizations of these domains in the art (though there is general agreement about the core of most of such gDDs). Precisely defining gD RBDs is similarly challenging. E.g., in HSV-1 gD RBDs for HVEM & Nectin-1 are known to partially overlap, but there also is ample evidence that FFs of gDs that do not bind HVEM can effectively, comparably, and in some cases improvingly bind Nectin-1 (See, e.g., Connolly S A, et al. J Virol. 2003; 77(14):8127-8140). As such, RBDs herein can be defined by WT AARSs and FVs thereof that are expected to be sufficient for binding to a referenced receptor, while additional residues (AAs/AARs) can in cases enhance such binding.

Applying this principle to defining the RBDs of HSV-1 gD (gDRBDs), a HVEM-binding domain (HVEMBD) herein means ˜AAs 28-48 (e.g., AAs 26-48). The Nectin-1 RBD (N1BD) of HSV-1 gD is less well defined, but herein means either residues 59-285 or a part thereof (e.g., a sequence defined by N-terminal residue ˜AA 59 and ending at about AA 235, 240, 245, 250, 260, 265, 268, 280, or 285. An overlapping domain that contributes to HVEM binding, N1 binding, or both, but which typically is not sufficient for binding either gDR independently is located in positions corresponding to HSV-1 gDs ˜AAs 49-58. The overlapping domain gDS can be AW a HVEMBD or N1BD (e.g., a N1BD can comprise AAs 48-285, 48-268, 48-265, 48-260, or 48-250. Functions of an overlapping domain comprise enhancing RBD(s) or to maintaining positioning of RBD(s) similar to positioning of WTC gDD(s) in WT gDP(s).

Regarding and exemplifying adapting HSV-1 gDS(s) to other gDD(s), in aspects gDP(s) comprise a PRV N1BD or a FV thereof corresponding to GASAOA (or being RVRHROSI/SVSHSOCE) to PRV AAs 19-350 or a fragment thereof such as AAs 20-340, 20-330, 20-300, 20-280, 20-260, or 20-250. In aspects, gDP(s) comprise a FV of a PRV N1BD in which AAs corresponding to MGASAOA of F29, W40, T137, Y201, Y216, M219-R220, P224 (or P224-Y226), V324, and Y237 of PRV gD are maintained.

gDS(s) that characterize gDP(s) exhibit 1+ defined function(s), such as receptor binding, checkpoint inhibition, ER processing promotion, placement of other gDDs, etc. However, gDS(s) of or related to a WT gDS can exhibit such properties or can exhibit such properties when combined with 1+ heterologous gDS(s) (e.g., a chimeric gDRBD that can bind gD receptor(s)), such that while only portion(s) of such a chimeric or chimeric-like gDS is RVRHRSIOI to any WT gDS it is a suitable gDS as it exhibits 1+ measurable gDS function(s). Functionality of a gDS FF or variant sequence (gDVS) can differ from a WT counterpart. In aspects, a gDS FF or gDVS exhibits suitable, comparable, or improved function(s). In aspects, a gDS FF/gDVS exhibits OSMOA of the functions of its WT counterpart. In aspects, a gDS FF or gDSVS exhibits <all functions of a corresponding WT AARS. Functions of gDS(s) include (1) gDSS functions; (2) receptor binding (e.g., nectin-1, nectin-2, or HVEM) (in gDRBD(s)) and target cell-binding (e.g., DCs, T cells, epithelial cells, or fibroblasts); (3) promoting uptake of the associated gDP (e.g., in a gDFP, such as a gDAgFP); (4) enhancing ER processing of the gDP; (5) enhancing GPI anchoring (6) membrane association (in a gD TMD); or (7) any other measurable function associated with gDD(s) or combinations of any such functions. As described elsewhere, gDP(s) can include non-functional WT gD AARSs or FVs thereof, but such AARS(s) do not characterize the gDP unless explicitly stated. gDP(s) also can comprise gDD(s) that are not discussed in detail herein but that exhibit function(s) (e.g., gDP(s) can be characterized on the inclusion of gDS(s) that DOS contribute or cause oligomerization/dimerization of gDP(s)). Typical functional and structural gDD(s) of gDPs are briefly discussed in turn below.

In aspects, gDPs comprise gD signal sequence(s) (gDSS(s)). In aspects, CEPs, gDPs, or both comprise 2+ gDSSs, such as in immature gDPs comprising two gDP portion(s) separated by SCS(s), or where a CEP comprises two different types of gDPs, such as 2+ gDPAgFPs. In aspects, CEPs or gDP(s) in CEPs comprise only 1 gDSS. In aspects, gDP(s) in CEPs do not comprise any gDSS. In aspects where no gDSS is in a CEP, gDP(s) can comprise heterologous signal sequence(s), such as those described elsewhere or are known in the art. gDPs can comprise any suitable type of gDSS. In aspects, gDPs comprise FLWT gDSS(s). In aspects, gDPs comprise a gDSS that is an FF of WT gDSS(s) or a chimeric gDSS. In aspects, gDPs comprise a gDSS that is a FV of either, such as GSRV gDSS(s). gDSS(s) are typically positioned in the N-terminus of an immature gDP and are typically directly attached to an adjacent sequence. In aspects, the adjacent sequence is a gDRBD sequence or an ETS. In aspects, gDPs comprising a gDSS comprise 1, 2, 3, 4, or more additional gDDs. In aspects, gDPs comprising a gDSS comprise no other gDDs. Examples of WT gDSs include MDV-gD AAs ˜1-34 (e.g., GenBank Q9E6L6.1/Uniprot Q6764); AAs˜1-17 of suid alphaherpesvirus 1 (e.g., GenBank: AA062939.1); AAs ˜1-19 of EHV-1 gD (e.g., Uniprot Q6DLD9); or AAs ˜1-18 of BHV-1 gD (e.g., Uniprot POCK29). In aspects, inclusion of the gDSS DOS enhances distribution of the associated PPT(s) in TR(s), level of IR(s), or both.

In aspects, OSMGAOA gDP(s) in CEPs comprise domain(s) capable of forming a hairpin structure in TR(s)/culture. In aspects, such a hairpin forming domain (HFD) is RVRHRSIOI, SVSHSOCE, or both to the WT HFD of HSV-1 gD (AAs ˜26-47). In aspects, in which a gDP comprises sequence(s) of or RVRHRSIOI/SVSHSOCE to a PFD or relevant portion thereof (a “displaced subdomain”), at least part of the HFD DOS is AW such a PFD AARS, at least when the gDP is not bound to gDR(s) in the case of gDPs comprising overlapping RBD(s) (e.g., a HVEM RBD). Determination of hairpin formation can be made by structural studies as described elsewhere (e.g., Connoly et al., 2003). In aspects, OSMGAOA gDP(s) in CEP(s) lack a functional HFD or lack any sequence that is RVRHRSIOI, SVSHSOCE, or both to the HFD of a WT gD. HFDs may be lacking/insufficient to serve as RBDs in other WT gDs. E.g., in PRV N-terminal loop of gD is only about half the size of HSV-1 gD, which has been attributed to the inability of PRV to bind HVEM. See, e.g., Li A et al. PLoS Pathog. 2017; 13(5): e1006314. In aspects, HFDs are positioned within about 85-125%, 90-110%, or 95-105% of the size of the HFD of HSV-1 gD. In aspects, a HFD is capable of binding HVEM suitably, comparably, or improvingly regarding to HSV-1 gD. In aspects, a HFD is not capable of DOS binding HVEM.

In aspects, OSMGAOA gDP(s) in a CEP comprise gD receptor binding domain(s) (gDRBD(s)). gDP(s) can comprise any suitable number of any suitable type(s) of gDRBD(s) that bind any suitable gD receptor (gDR). A gDRBD herein is characterized as the minimum AARS that is sufficient to exhibit at least suitable/adequate, and typically at least comparable binding to a gD receptor (gDR) as a WT gD. In aspects, gDP(s) can comprise a single AARS that comprises multiple gDRBDs. For example, a gDP can comprise AAs ˜26-280 of HSV-1 gD which comprise most/all of all three RBDs in HSV-1 gD or an FF/FV thereof that exhibits multiple receptor binding function(s).

In aspects, gDP(s) in CEPs bind ≥2 gDRs. In aspects, gDP(s) bind only a single gDR (e.g., only HVEM, only nectin-1 (N1), or only nectin-2 (N2)). In aspects, the only gD receptor gDP(s) bind is N1, N2, or HVEM. In aspects, gDP(s) bind N2 and N1, but not HVEM. In aspects, gDP(s) bind N2, but not N1 and not HVEM. In aspects, gDP(s) bind N1, but not HVEM or N2. In aspects, gDP(s) at least adequately or comparably bind 3OSHS. In aspects, gDP(s) do not bind 3OSHS. In aspects, gDP(s) bind 1+WT gDR(s) other than HVEM, a nectin, or 3OSHS. In aspects, gdP(s) bind MHC gDR(s).

gDP(s) can comprise gDRBD(s) of or that are RVRHRSIOI/SVSHSOCE to WT gD RBD(s) of any suitable α-HV gD or FFs thereof, and gDP(s) can comprise functional chimeric RBD(s). Most a-HV comprise gDs (a notable exception is human alphaherpesvirus 3 (HHV-3), usually referred to as the varicella-zoster virus (VZV)). Most gDs bind either HVEM or a HVEM/homolog, a Nectin (e.g., a Nectin-1 (e.g., HSV-1, HSV-2, PRV, SuHV-1, BHV-5, and BHV-1), Nectin-2 (PRV, HSV-2, and mutant HSV-1), or nectin-like-5/CD155 (e.g., PRV and BHV-1), or 3-O-sulphated heparan sulphate receptor (“3OSHS”) (e.g., HSV-1, but not BHV-5) (See, e.g., Krummenacher C et al in Madame Curie Bioscience Database. Austin (TX): Landes Bioscience; 2000-2013 and Levings, R., graduate thesis, Iowa State Univ., 2012). The HSV-1 gDRBDs for Nectin-1, HVEM, and 3-O—S HS are described elsewhere, as are select examples of RBDs for such receptor(s) in other uHV gDs. In aspects, gDS(s) comprise other gD RBD(s). E.g., an EHV-1 gDRBD, EHV-4 gDRBD, or RVRHROSI FV gDRBD can comprise an MHC I RBD. See Azab W et al. J Virol. 2012; 86(4):2031-2044 and Spear P G, et al. Virology. 2006; 344(1):17-24. In aspects, OSMGAOA gDP(s) in CEPs lack any gDRBD. In aspects, OSMGAOA gDP(s) in CEPs comprise ≥1 gDRBDs, ≥2 gDRBDs, or ≥3 gDRBDs. In aspects, OSMGAOA of the gDRBDs comprise WT gDS(s). In aspects, OSMGAOA of gDS(s), such as gDRBD(s), in EP(s) are RVRHROSI to gDS(s)/gDRBD(s) of the same uHV. In aspects, ≥2 gDRBDs in a CEP are from WT gDS(s) or are RVRHROSI to gDRBDs of two different species of uHV (e.g., PRV and HSV-1).

In aspects, OSMGAOA gDP(s) in CEPs bind HVEM with a suitable, comparable, or improved affinity as compared to a corresponding WT HVEM-binding gDP (e.g., HSV-1 gD) and, accordingly, in aspects OSMGAOA of gDP(s) in a CEP comprise ≥1 HVEM-binding gDRBD(s). In aspects, OSMGAOA of such gDP(s) exhibit CI activity in HVEM-expressing TRs. In aspects, a HVEM-binding RBD is identical to a WT gDRBD. In aspects, a HVEM-binding RBD is a FV that is RVRHROSI to a WT HVEM-gDRBD. E.g., in one aspect a HVEM-gDRBD is a FV of an HSV-1 HVEM-gDRBD that comprises a variation at AA 22. In aspects, a CEP comprises gDP(s) comprising HSV-1 gD HVEM-gDRBD FVs in which G47, Y48, P42, K51, Q52, and G59 are maintained, and optionally further in which OSMGAOA of AAs 36, 37, 40, 53, and 54 is maintained. In aspects, GAOA of AAs 32-40, 49-57, or both of HSV-1 are maintained. In aspects, HSV-1 gD AA 27 39, or 47 are also/alternatively maintained, with such residues DOS contributing to HVEM binding. In aspects, CEPs comprise a FV HVEMBD in which OSMGAOA of the AAs in positions 43, 45, 46, and 49, and OSMGAOA of the residues in overlapping domain AAs 50, 55, 56, 57, or 58 (L, K, S, P, G, E, L, or T, respectively) are substituted with a suitable substituting AA (e.g., an Ala). In aspects, AAs 101, 99, 64, 61, and 60 in the Nectin-1 RBD also are maintained and DOS enhance HVEM binding of such a gDP. In aspects, a FV HVEMBD comprises a sequence according to the formula of SEQ ID NO: 719. In aspects, no more than 75% (6/8), no more than 50% (4/8), or nor more than 3, 2, or 1 of such X AAs varies from corresponding AA(s) in HSV-1 gD.

In aspects, a gDP comprising a HVEM binding domain (HVEMBD) lacks SMGAOA of any gD PFD (e.g., most, generally all, substantially all, or all of any residues corresponding to residues 315-324, 310-324, 315-394, or AAs 310-394 of HSV-1 gD). In aspects, such gDP(s) exhibit significantly enhanced affinity for HVEM than WT HSV-1 gD (e.g., at least 2×, 5×, 10×, 20×, 50×, 75×, or 100×greater affinity). In aspects, a gDP comprises a gDS in which MGAOA of such a portion of the PFD is maintained but in which one or more substitutions are introduced that DOS enhance HVEM affinity (e.g., substituting W319 with a different residue, e.g., Ala). In aspects, such gDP(s) exhibit significantly improved KO_{N} values as compared to WT HSV-1 gD with respect to HVEM binding. In aspects, any such gD may maintain portion of a PFD or adjacent sequence comprising, e.g., AAs 259-300 of HSV-1 gD or a RVRHROSI sequence. In aspects, such gDP(s) exhibit a significantly lower K_{off} than gDP(s) lacking such a sequence. In aspects, a HVEMBD gDP that comprises a core domain comprises substitution(s) at AAs corresponding to AAs 165, 240, 247, 248, or 256 of HSV-1 gD, in aspects at least 2 or 3 thereof, the substitutions resulting in DOS enhanced HVEM binding as compared to WT HSV-1 gD.

In aspects gDP(s) comprising a HVEMBD & a PFD do not include any insertions of ≥12, ≥10, ≥8, ≥7, more than 6, more than 5, or more than 3 residues between the HVEMBD and PFD. In aspects, gDP(s) comprising a HVEMBD & a PFD comprise no insertions that DOS reduce HVEM binding.

In aspects, a gDP exhibits a KD of about 0.5-6.5 (e.g., ˜2-6)×10⁻⁶ M for HVEM, a Kₒₙ of about 3-150 (e.g., ˜50-150)×10³ M with respect to HVEM, a K_{off} of 1-6 (e.g., ˜3-6)×10⁻² M with respect to HVEM, or a combination thereof. Methods for assessing HVEM binding are described in, e.g., Rux A H et al. Journal of Virology. 1998 September; 72(9):7091-7098; Lazear E et al. Virology. 2014; 448:185-195; etc. In gDP(s) lacking a functional HVEMBD, such gDP(s) can exhibit DOS lower affinity characteristics than any of these affinities.

In aspects, gDP(s) of CEPs comprising a HVEMBD(s) exhibit DOS gD CI effects in HVEM-expressing TRs (“HVEM TRs”). In aspects, gDP(s) bind HVEM with at least comparable affinity as BTLA or a BTLA homolog binds HVEM. In aspects, gDP(s) bind HVEM with DOS greater affinity than exhibited by BTLA PPTs in cells or TRs (or population of similar cells or TRs, e.g., as determined by a statistically significant population in an in vivo or in vitro study). In aspects, such gDP(s) in CEPs DOS induce enhanced antigen specific IR(s).

In aspects, CEPs comprise LIGHT PPT(s) that DOS enhance(s) IR(s). In aspects, methods comprise the use of such LIGHT PPT(s) or other stimulators of a HVEM-mediated checkpoint pathway as A. In aspects, CCs comprise such LIGHT PPT(s) or HVEM pathway activator(s) as CCCs.

In aspects, OSMGAOA of gDP(s) in a CEM comprising HVEMBD(s) comprise less than an all of the gDD(s) in any WT gD(s) related to the HVEMBD(s) (e.g., in aspects gDP(s) comprise that comprise an HSV-1 HVEMBD or a FV thereof and lack one or more other domains corresponding to the other domains of HSV-1 gD, such as a nectin-1 RBD, a PFD, a TMD, or an intravirion domain. In aspects, CEPs comprise gDP(s) that consist or consist essentially of HVEM RBD(s) (WT or FV) or in which the only gDD(s) are HVEMRBD(s).

In cases, OSMOA gDP(s) in CEPs lack any HVEMBD that binds HVEM in a comparable/better manner (e.g., a reduction of 50%, 75%, 90% or more) (e.g., an HSV-1 gD lacking AAs 32-40 or suitable substitutes therefore may exhibit a ˜90% reduction in HVEM binding). In cases, OSMGAOA gDP(s) in CEPs lack any gDD that exhibits significant binding of HVEM, HVEM-mediated checkpoint inhibition in TR(s), or both. In cases, gDP(s) in CEPs do not exhibit HVEM-mediated checkpoint inhibition, significant HVEM binding, or both. In cases OSMGAOA gDP(s) in CEPs lack any gDS(s) including AARS(s) that is related/similar to any WT gD HVEMBD.

In aspects, gDP(s) in CEPs comprise Nectin-1 binding domain(s) (N1BD(s)). In aspects, gDP(s) comprise ELWT gD N1BD(s). In aspects, gDP(s) comprise FFs of such gDS(s). In aspects, gDP(s) comprise chimeric N1BD(s) (e.g., a PRV & HSV-1 N1BD). In aspects, gDP(s) comprise FV(s) of a WT N1BD AARS that is RVRHROSI to a WT gD N1BD (e.g., HSV-1 gD, BHV-1 gD, or PRV gD). In aspects, a FV N1BD is RVRHROSI to the WT HSV-1 gD N1BD. In aspects, a FV N1BD retains AAs corresponding to Tyr63, Asp240, Arg247, and Phe248 of HSV-1 gD or AAs that correspond in function, position, or both.

In aspects, gDP(s) exhibit an affinity for Nectin-1 that is comparable or superior to the affinity between CD96 and Nectin-1 in TR(s). In aspects, gDP(s) exhibit affinity for Nectin-1 DOS ≤˜the affinity of CD155.

In aspects, gDP(s) comprise a sequence that is RVRHRSIOI, SVSHSOCE, or both to SEQ ID NO:56 (AAs 58-255 of HSV-1 gD). In aspects, gDP(s) comprise a gDS that is RVRHRSIOI, SVSHSOCE, or both to SEQ ID NO:57 (residues 58-269). In aspects, gDP(s) comprise a gDS that is RVRHRSIOI, SVSHSOCE, or both to SEQ ID NO: 72 (residues 58-290), SEQ ID NO:71 (AAs 58-302); SEQ ID NO:58 (residues 58-313); or SEQ ID NO:73 (AAs 58-340). In aspects, gDP(s) comprise a gDS that is RVRHRSIOI, SVSHSOCE, or both to SEQ ID NO:53 (residues 55-255 of HSV-1 gD); SEQ ID NO:54 (residues 55-269); SEQ ID NO:55 (residues 55-313); or SEQ ID NO:69 (AAs 55-340). In aspects, gDP(s) include a gDS that is RVRHRSIOI, SVSHSOCE, or both to SEQ ID NO:50 (residues 48-255 of HSV-1 GD); SEQ ID NO:51 (residues 48-269); SEQ ID NO:64 (residues 48-302); SEQ ID NO:52 (residues 48-313); or SEQ ID NO:64 (residues 48-340). In aspects, gDP(s) comprise homologous N1BD sequence(s) from a non-HSV-1 gD.

In aspects, gD(s) comprise both N1BD(s) and HVEMBD(s). In one such aspect, gDP(s) comprise a gDS that is RVRHRSIOI, SVSHSOCE, or both to SEQ ID NO:40 (residues 26-255); SEQ ID NO:35 (residues 26-269); SEQ ID NO:45 (residues 25-313); or SEQ ID NO:82 (residues 26-340). In aspects, gDS(s) comprise a HVEM1BD, N1BD, or both, which are RVRHRSIOI/SVSHSOCE to homologous sequences from a non-HSV-1 WT gD.

In aspects, a first mature gDS (a gD1) of a mature gDP, PCGCOSCOOCO a HVEMBD, a N1BD, or combined HVEMBD&N1BD gDS, and the gDP comprises one or more gDS(s) downstream of such gD1 sequence (e.g., a gD2 sequence). E.g., in aspects, a gDP comprises an above-described HSV-1 gD or HSV-1 gD related HVEMBD, N1BD, or combined HVEMBD-N1BD gDS as a gD1 sequence and a second gDS (a gDS-2 sequence) that is RVRHRSIOI/SVSHSOCE to a portion of a WT gD, such as HSV-1 gD, that begins at a position corresponding to the position following the C-terminus of the gDS in gD-1 or a position that is 2, 3, 4, 5, 6, 7, 8, 9, 10, 12, 15, 16, 18, 20, 22, or 25 residues downstream of such residue, and that ends at a position corresponding to HSV-1 gD residue 394, residue 375, residue 360, or residue 350, residue 340, residue 335, residue 315, residue 310, residue 308, residue 300, residue 290, or residue 285 of HSV-1 gD. In aspects, such a gDP is a gDFP comprising one or more intervening AARSs positioned between the gD1 and gD2 portions of the gDP. In aspects, the inclusion of such intervening sequences does not prevent the gDD(s) of the gDP from exhibiting suitable, comparable, or improved functionality as compared to the WT counterpart gDD(s). In aspects, such a gDFP is a gDAgFP comprising one or more Ag sequences positioned between gD1 and gD2, such as at least 2, 3, 4, 5, 6, 8, or 10 Ag(s), which are optionally AW PTPS(s), SCS(s), FL(s)/MSL(s), or CT.

In aspects, CEPs comprise gDAgFP(s) comprising a gD1 as described above (e.g., SEQ ID NO:57 (residues 58-269 of HSV-1 gD); SEQ ID NO: 72 (residues 58-290), SEQ ID NO:71 (residues 58-302); SEQ ID NO:58 (residues 58-313); or SEQ ID NO:73 (residues 58-340), or a FF or FV of any thereof) and that further comprise a gD transmembrane domain (e.g., residues 341-361 of HSV-1 gD or a homologous sequence or a FF or FV of either) or cytosolic/ectodomain (e.g., residues 361-394 of HSV-1 gD or a homologous sequence or a FF or FV of either) (e.g., SEQ ID NO:66 (corresponding to residues 48-394), SEQ ID NO:83 (corresponding to mature HSV-1 gD, residues 26-394), or SEQ ID NO:70 (corresponding to residues 55-394 of HSV-1 gD). In aspects, Ag(s) can be directly or indirectly bound to the gD sequence upstream of the gD sequence(s), downstream of the gD sequence(s), or both. In aspects, OSMGAOA Ag(s) in the gDP are downstream of any gDS in the gDP.

In aspects, gDP(s) comprise an intervening heterologous AARS between a HVEMBD and a N1 BD. In aspects, such an insertion occurs at a position corresponding to residue 47 of HSV-1 gD.

In aspects, any insertions of heterologous AARSs in a N1BD do not eliminate suitable or comparable Nectin-1 binding. In aspects, any such insertions do not DOS reduce N1 binding. In aspects, no heterologous AARS of more than 15 AAs, no more than 10 AAs, no more than 5 AAs is inserted between any gDS that is RVRHRSIOI to AAs 59-240 of HSV-1 gD in a gDP.

In aspects, a gDP binds Nectin-1 with at least 1.5×, at least 2×, at least 2.5×, at least 3×, at least 4×, or at least 5×the affinity of WT HSV-1 gD (e.g., at least 7, 8, or 9×the affinity). In aspects, such a gDP comprises a Nectin-1 binding domain that is more related to the N1BD of PRV gD than HSV-1 gD. Aspects of determining affinities and features of PRV, BHV-1, and HSV gDs are described in, e.g., Connolly 2001, supra. Nectin-1 binding characteristics are described in Li A et al. PLoS Pathog. 2017; 13(5): e1006314.

Other N-terminal first sequence (gD1) and C-terminal (gD2) second sequence combinations that can be for internal antigen sequence gD fusion protein constructs that can be used in compositions and methods are provided in the Wistar Art (See, e.g., Col. 8 of the '816 patent). In aspects, the gDP comprises gD1 and gD2 sequences that is not described in the Wistar Art (e.g., by inclusion of different residues, smaller gDs that are FFs, or FVs not contemplated in the Wistar Art). In aspects, gDP(s) lack one or more residues or sequences associated with HVEM binding, lack a gD intravirion/cytosolic domain, lack a TMD, comprise Ag(s) that are positioned downstream of any gDS in the FP, initially comprise a gDSS, or comprise combinations thereof.

In aspects, gDP(s) comprising a N1BD lack a gD TMD & cytosolic domain. In aspects, such gDP(s) exhibit DOS higher affinity for Nectin-1 than gDP(s) with such gDDs. In aspects, gDP(s) comprising a N1BD, HVEMBD, or both, lack any gDS corresponding to residues 310-394 of HSV-1 gD. In aspects, such gDP(s) exhibit DOS enhanced binding for one or both gD receptors. In aspects, such gDP(s) exhibit at least 10×, at least 20×, at least 30×, or at least 50×affinity for Nectin-1 than WT HSV-1 gD.

In aspects, a gDP comprises a heterologous ETS upstream of a N1BD, in essence taking the place of the HVEMBD in HSV-1 gD. E.g., a DEC-205-binding domain can be located in such a position.

In aspects, the N1BD comprises up to about residue 250 of HSV-1 gD or a corresponding residue of a homolog or FV. In aspects, residues 240, 247, and 248 of HSV-1 gD are maintained as part of the N1BD. In aspects, such gDP(s) exhibit DOS enhanced binding of Nectin-1 due to the presence of such residues. In aspects comprising a HVEMBD or 3OSHSBD, such residues are deleted, or such a section is deleted/not present (e.g., residues corresponding to AAs 240-250 of HSV-1 gD are deleted,) and the associated gDP exhibits DOS enhanced binding of HVEM, 3OSHS, or both.

In aspects, gDP(s) comprise a FV of an HSV-1 gD N1BD. In aspects, SMGAOA of the AAs corresponding to R61, V62, Y63, H64, Q157, R159, and V239, of HSV-1 gD are retained (in this disclosure WT gD AAs are counted from the first residue of the gDSS). In aspects, SMGAOA of AAs S241, 1242, G243, M244, L245, N252, T255, V256, and Y259 also are retained. In aspects, all of V2229-V248 are retained. In aspects, one, some, or all (OSOA) of P48, L50, and Q52 are also retained. In aspects, Q101, N102, and M110 are retained. In aspects in which a gDP comprises HVEMBD and N1BD sequences at least related to HSV-1 gD counterparts both L50 and Q52 are retained & both residues contribute to both Nectin-1 and HVEM binding. In aspects, gDPs comprise an HSV-1 N1BD FV that comprises substitutions at residues corresponding to OSMOA of T91, N107, S109, or F154. In aspects, gDPs comprise N1BD FVs that has substitutions at positions corresponding to HSV-1 AAs 59, 240, 247, or 248. In aspects, AAs corresponding to OSMOA of HSV-1 gD AAs 107, 109, 154, 240, 247, or 248 are retained.

Exemplifying adapting such principles to other aHV gDS(s) and FFs, in aspects gDP(s) comprise an HSV-2 gD N1BD (HSV-2 gD AAs 48-258). In aspects, gDP(s) comprise a FV of an HSV-2 N1BD that is RVRHROSI/SVSHSOCE to HSV-2 N1BD & that retains AAs corresponding to MGAOA of HSV-2 gD AAs P48, L50, D51, Q52, R61, V62, Y63, H64, Q157, R159, P223, V229, D240, S241, 1242, G243, M244, L245, P246, R247, F248, N252, V256, & Y259.

In aspects, a gDP comprises an N1BD that comprises sequences that are VRHRSIOI to WT Nectin-1 BD patch 1 and patch 2 sequences or that are VSHSOCE and, optionally, within about 5 angstroms of the patch 1 and patch 2 nectin-1 binding domains of HSV-1 gD or HSV-gD when structurally aligned (e.g., HSV-2 gD patch 1 residues comprising P23, L25 to Q27, F223, N227, V231, and Y234 and HSV-2 gD patch 2 residues comprising R36 to H39, Q132, R134, P198, and V214 to R222).

In aspects, gDPs comprise a N1BD sequence according to FORMULA 2 in US20200325182 (SEQ ID NO:745). In aspects, a gDP comprises a sequence according to FORMULA 3 of US20200325182 (SEQ ID NO: 746). In aspects a gDP further comprises a sequence downstream of position X122 thereof according to the formula Xa-C₅—X(1)-X(2)-Xb-X(3)-C₆, wherein Xa is any 6 to 8 residues; X(1) is Y or F; X(2) is A or S; Xb is any 7 to 11 residues; and X(3) is A, L, or W (FORMULA 4; SEQ ID NO:747). In aspects, one or more of C and C₅, C₂ and C₆, and C₃ and C₄ in gDP(s) of such formula(s) form cysteine-cysteine double bonds. In aspects, all of C1 and C₅, C₂ and C₆, and C₃ and C₄ form cys-cys double bonds. In aspects, gDPs comprise a sequence according to any of the above-described formulas where the gD sequence further comprises a sequence downstream of the Formula 4 sequence according to FORMULA 5 of US20200325182 (SEQ ID NO:748). In aspects, the Formula 5 sequence has a sequence according to FORMULA 6 of US20200325182 (SEQ ID NO:749). In aspects, the Formula 6 sequence further comprises a sequence according to FORMULA 7 of US20200325182 (SEQ ID NO: 750). In aspects, a gD sequence further comprises a sequence downstream of the Formula 4 FORMULA 8 of US20200325182 (SEQ ID NO:751). In aspects, the gDD sequence comprises a sequence upstream of the Formula 1, Formula 2, or Formula 3 sequence according to FORMULA 9 of US20200325182 (SEQ ID NO: 752). In aspects, the Formula 9 sequence comprises a sequence according to FORMULA 10 of US20200325182 (SEQ ID NO: 753). In aspects, the Formula 9 sequence comprises a sequence according to FORMULA 11 of US20200325182 (SEQ ID NO: 754). In aspects, a gDP comprises a sequence according to any of Formulas 1-3 further includes sequence upstream of the Formula 1-3 sequence according to FORMULA 12 of US20200325182 (SEQ ID NO:755).

Features of this section overlap with the description of other sections below, particularly the core domain and N-terminal and C-terminal extensions thereof. In aspects, the N-terminal extension region of a core domain is materially, mostly, generally, or at least substantially composed of flexible AAs. Additional aspects relating to N1BDs, variants, and related methods and principles adaptable to such aspects are described in, e.g., Manoj S et al. PNAS 2004; 101(34):12414-12421; Martinez W M et al. J Virol. 2002; 76(14):7255-7262; and Alves Dummer L et al. Vet Res. 2014; 45(1):111.

HVEMBD gDPs, N1BD gDPs can comprise “deletions” (truncations/omissions), substitutions, or both, of gDSs/AAs downstream of the N1BD. In cases, such omissions/deletions or substitutions DOS enhance Nectin-1 binding to the gDP. In aspects, substitutions are made in AAs corresponding to HSV-1 gD AA 150, 151, 176, or 302. In aspects, gDP(s), such as N1BD, HVEMBD, or HVEMBD+N1BD gDP(s) lack AAs corresponding to HSV-1 gD AAs 305-325, 310-325, 305-335, 310-335, 305-340, 310-340, 305-361, 310-361, 305-394, or 310-394 or corresponding AAs from a homolog related to gDS(s) of the gDP, e.g., a N1BD. In aspects, gDP(s) lack W319, e.g., through substitution or deletion. In cases, such gDP(s) exhibit DOS better affinity for receptor(s). In cases, gDP(s) comprise a gDS corresponding to HSV-1 AAs 260-305, 280-305, or 285-305, but lack an AARS corresponding to AAs 310-335, 310-360, or 310-394.

In aspects, CEPs comprise gDP(s) comprising an N1BD that DOS competes with a TR Nectin-1 for binding to WT Nectin-1 (and DOS prevents Nectin-1:Nectin 1 dimer formation in some cases). In aspects, gDP(s) exhibit DOS better affinity for Nectin-1 than WT Necting-1. In aspects, gDP(s) exhibit at least 2×, 3×, 4×, 5×, or ≥10×, e.g., at least 20×, at least 30×, at least 50×, or at least 100×or higher affinity for Nectin-1 than Nectin-1.

In aspects, gDP(s) exhibit a Ka for Nectin-1 of at least 10 nM, at least 15 nM, at least 20 nM, or at least 25 nM. In aspects, gDPs exhibit a Ka for Nectin-1 of at least 50 nM, e.g., at least 100 nM, at least 120 nM (e.g., 110 nM-360 nM), at least 150 nM, at least 175 nM, at least 190 nM, at least 200 nM (e.g., at least ˜250 nM, at least 275 nM, ≥300 nM, ≥˜330 nM, at least 350 nM, at least 380 nM, or at least 400 nM), such as 15-450 nM, 15-300 nM, 15-350 nM, 30-390 nM, 50-350 nM, 100-350 nM, or 20-400 nM).

In aspects, gDP(s) exhibit a Nectin-1 Kₒₙ of at least 1.0 ×10⁵ M⁻¹s⁻¹, 1.25×10⁵ M⁻¹s⁻¹, 1.5×10⁵ M-'s⁻¹, 2×10⁵ M⁻¹s⁻¹, 2.5×10⁵ M⁻¹s⁻¹, 3×10⁵ M⁻¹s⁻¹, 4×10⁵ M⁻¹s⁻¹, 5×10⁵ M⁻¹s⁻¹, or even at least 7 ×10⁵ M⁻¹s⁻¹) (e.g., 1-10, 1.2-8.8, 1.5-7.5, 2-8, or 2-10×10⁵ M⁻¹s⁻¹-1.25×10⁵ M⁻¹s⁻¹). In aspects, gDP(s) exhibit a Nectin-1 K_{off} of at least 0.5, 0.7, 1, 1.1, 1.25, 1.5, or 2 (e.g., 0.5-3, 0.5-2.5, 0.7-3.5, 0.7-2.8, 1-4, 1-3, or 1-25)×10⁻² s⁻¹, 1.17×10⁻² s⁻¹. In aspects, gDPs exhibit at least 1.5×, at least 2×, at least 2.5×, or at least 3×enhanced K_{d}, K_{off}, or Kₒₙ values as compared to WT gD(s), such as HSV-1 gD, HSV-2 gD, or PRV gD. Methods for assessing such values and relevant modifications of PRV gD that can be adapted to such aspects are provided in Li A et al. PLoS Pathog. 2017; 13(5): e1006314. Other modifications to gDS(s), methods for evaluating such modifications, and related compositions/methods adaptable to aspects are described in references cited in US20200325182.

In aspects, gDP(s) comprise N1BD(s), HVEMBD(s), or CT, which exhibit suitable, comparable, or improved affinity characteristics (K_{d}, K_{off}, or Kₒₙ values) for NIs, HVEMs, or combination thereof (“CT”), of ≥2 species (e.g., swine N1 and human N1) as compared some, most, or all (SMOA) of the gDs of a-HVs that typically infect some, most, most, or all (SMOA) of such TRs. E.g., in aspects, gDPs of CEPs exhibit better affinity for human N1, swine N1, or both, as compared to HSV-1 gD, HSV-2 gD, PRV gD, or a combination thereof. In aspects, gDP(s) exhibit affinity characteristics that are suitable, comparable, or improved with respect to at least two or more types of NIs of different TR species as compared to the gDs of a-HVs that typically infect such TRs. E.g., gDP(s) can exhibit at least suitable or at least comparable binding to swine N1 & human N1 with respect to HSV-1 gD & PRV gD, respectively.

In aspects, gDP(s) comprise a Nectin-2 (N2) RBD (N2RBD). In aspects, gDP(s) comprise a WT N2RBD. In aspects, gDP(s) comprise a variant of a non-N2-binding gD (e.g., a variant of HSV-1 gD) that is modified to be able to at least adequately bind N2. E.g., in aspects, gDP(s) comprise a gDS comprising generally all or most of the C-terminal portion of the HSV-1 gD HVEMBD (but not necessarily enough to comparably or adequately bind HVEM), the overlapping domain, and the N1BD but that include a substitution at position L50, L53, Q52, or combinations in the overlapping domain (e.g., Q52R, Q52P, or Q52A; L50P; or L53A). In aspects, gDP(s) including N2BD(s) include an AARS RVRHRSIOI/SVSHSOCE to AAs 32-57 of HSV-1 gD/HSV-2 gD.

In aspects, such gDP(s) adequately, comparably, or improvingly bind both N2 and N1, but not HVEM (e.g., a Q52 variant); N2 and HVEM; N1, N2, and HVEM (e.g., a L53 variant); or only N2. In aspects, such gDP(s) adequately, comparably, or improvingly bind 30SH. In aspects, gDP(s) that bind N2 do not bind 3OSHS. In aspects, gDP(s) comprising a N2BD also suitably or at least comparably bind 3OSHS. As described elsewhere, in aspects variants gDD(s), such as N1BD, N2BD, HVEMBD, or combinations that comprise gDSV(s) exhibit structural similarity to corresponding/counterpart gDD(s). E.g., a substitution at L50 comprises a geometrically constrained AA, such as a Pro, versus a more flexible AA, such as a Gly or Ala, which may be required to maintain sufficient structure of an N2BD to permit at least suitable N2 binding. In aspects, gDP(s) comprise an N2BD that binds to N2 PPTs of two or more species (e.g., mice and humans). Aspects of N2BDs, including variants, that can be adapted to such aspects are described in Connoly et al., 2003, supra and Landsburg D J et al. J Virol. 2003; 77(14):8127-8140.

In aspects, OSMGAOA gDP(s) of CEPs lack any N2BD that DOS (1) enhance immune system NKC downregulation/evasion (e.g., decrease NKC degranulation, lysis, or both of DCA-cells), (2) impair DNAM⁻¹ binding, or (3) degrade endogenous N2. Effects of gDP(s) on such functions in NKCs are described in, e.g., Grauwet K et al., PNAS 2014, 111 (45) 16118-16123; DOI: 10.1073/pnas.1409485111. In aspects, OSMGAOA gDP(s) lack WT N2BD(s). In aspects, OSMGAOA gDP(s) lack any N2BD(s).

In aspects, gDP(s) comprise RBD(s) that DOS bind to gDR(s) besides N1, N2, and HVEM. In aspects, such gDR(s) comprise 3OSHS, an MHC gDR, or a nectin family gDR other than N1 or N2. In aspects, gDP(s) bind two or more of such alternative gDR(s). In aspects, such gDP(s) also bind one, two, or all of N1, N2, and HVEM. In aspects, such alternative gDR-binding gDP(s) bind less than all, only one, or none of N1, N2, and HVEM.

In aspects, gDP(s) bind 3OSHS. In aspects, gdP(s) bind 3OSHS suitably, comparably, or better than HSV-1 gD. In aspects, gDP(s) bind both 3OSHS and N1. In aspects, gDP(s) bind N1, but not 3OSHS. In aspects, gDP(s) bind 3OSHS and comprise a gDRBD for the gDR that is RVRHRSIOI/SVSHSOCE to HSV-1 gD AAs 26-57. In aspects, gDP(s) that bind 3OSHS comprise a gDS that is RVRHRSIOI/SVSHSOCE to HSV-1 gD AAs 26-285, 26-340, or 26-265. In aspects, such a gDS retains AAs corresponding to at least half MGAOA of K26, R60, R61, R155, K147, and K215 of HSV-1 gD. In aspects, gDP(s) bind 3OSHS with DOS greater affinity characteristics than HSV-1 gD. In aspects, such gDP(s) comprise substitutions at two or three of AAs corresponding to AAs D240, R247, and F248 of HSV-1 gD. Aspects of gD interactions with 3OSHS adaptable to aspects are described in Yoon M et al. J Virol. 2003; 77(17):9221-9231. In aspects, a gDP exhibits reduced binding of 3OSHS and comprises substitutions or deletions at 1+ AAs corresponding to HSV-1 gD AAs L50, L53, Q52, or combinations, which result in such reduced 3OSHS binding. In aspects, gDP(s) exhibit a Ka for 3OSHS of less than about 1×10⁻⁶ M, such as ≤˜1×10⁻⁷ M or ≤˜1×10⁻⁸ M. In aspects, gDP(s) exhibit a Ka for 3OSHS of about 0.5-9.5×10⁻⁶ M. In aspects, gDP(s) exhibit a Ka for 3OSHS that is ≥1×10⁻⁵ M, such as at least 5×10⁻⁵ M or at least 1×10⁻⁴ M.

In aspects, gDP(s) comprise a gDD that corresponds to MGAOA of a WT gD core domain. In aspects, a core domain exhibits RBD functionality, e.g., is a N1BD or N2BD. In aspects, gDP(s) comprise a core domain (gDCD) that comprise a portion that DOS enhances the functioning of RBD(s). In still other aspects, gDCD(s) primary function comprises or is to maintain the spatial relationship of other domains (e.g., an HVEMBD and a partial or total PFD).

Typically, gDCDs comprise an immunoglobulin (Ig)-like V-fold structure (e.g., in a sequence corresponding to HSV-1 gD AAs ˜80-210) (an “IgV domain” or IgVD). Typically, gDCDs also comprise an N-terminal flanking domain (CDNTFD) (corresponding to HSV-1 gD AAs ˜59-79 or a FV, FF, or homolog thereof); C-terminal flanking domain (CDCTFD) (corresponding to HSV-1 gD AAs ˜211-280 or a FV, FF, or homolog thereof); or both such flanking domains. Such structures are common to a-HV gDs. E.g., PRV gD exhibits such a structure, as does HSV-1 gD and HSV-2 gD.

In aspects, gDCD(s), such as variants of a gDCD, have structural elements or a local or global structure that is similar to WT gDCD(s). In aspects the gDCD comprises MGASAOA of the six cysteine residues that form three cysteine bonds in many WT gDs, including HSV gDs (e.g., cys residues corresponding to HSV-1 gD AAs and disulfide bonds cys91: cys214, cys131: cys227, and cys143: cys152). In aspects, the position of each such corresponding cys residue in a gDD differs by less than about 20 residues, 15 residues, 10 residues, 8 residues, or 5 residues (e.g., 0-3 residues, 0⁻⁵ AAs, 0-7 residues, 0-10 residues, 0-12 AAs, 0-16 AAs, or 0-25 residues) regarding the corresponding position of such cys residues in a related WT gD when aligned.

In aspects, the IgVD comprises a nine-stranded central 0-barrel structure. In aspects, the IgVD comprises a kinked, e.g., middle-kinked C″ strand, in cases leading to 2 strand halves connected with a distorted loop. In aspects, the gDCD contains two α-helices (α1 and α1′), located between strands C″ and D or between the BC and the C″D strands of the gDCD. In aspects, a CDCTFD comprises or does not comprise an additional α2′ helix.

In aspects, a gDD variant sequence comprising a gDCD and optionally related N-terminal or C-terminal extensions will exhibit or be predicted to comprise an IgV domain through empirical structural analysis (e.g., crystallography methods), computer-aided sequence analysis, or both. In aspects, gDV(s) differ from WT gDS(s) when structurally aligned by r.m.s. deviations of between ˜0.1 and 0.9 (e.g., 0.3-0.9), 0.1 and 0.75, or 0.1-0.5 Å in SMGASAOA of the compared gDD(s). PMCs are provided in US20200325182.

In aspects, gDP(s) comprise a gDCD-like sequence that when analyzed by computational sequence structure analysis, such as by PFAM/Interpro Scan analysis, is identified as being associated with an immunoglobulin-like domain superfamily sequence (e.g., InterPro entry IPR036179) with an E-value that indicates the structure is likely present, very likely present, highly likely present, or almost certainly present in the variant. In aspects, gDP(s) comprise a variant gDCD that is identified as exhibiting a Herpesvirus glycoprotein D/GG/GX domain family using a tool trained to identify the presence of this domain in a sequence, such as the NCBI Conserved Domain Database (“CDD”), Interpro Scan/PFAM, Motif Scan, or the EMBL Xfam sequence search tool. In aspects, the variant will exhibit an E-value, bit score, or both, which indicates that the presence of such a domain in the variant is likely, very likely, highly likely, or almost certain. E.g., an E-value ≥10e-100 or in aspects an E value between 10e-30 and 10e-100, 10e-10 and 10e-30, or 10e-6 and 10e-10. A portion of HSV-1 gD (residues 82-206), for example, exhibits an E-value of 1.72 e-45 with respect to pfam01537 and HSV-2 exhibits an E-value of 8.0e-39 with respect to its relationship to pfam01537.

In aspects, a variant gDCD comprises less than 5, less than 4, less than 3, or 1-2 insertions in the IgVD or overall gDCD. In aspects, a variant gDCD comprises no insertions regarding a WT IgVD or a WT gDCD. In aspects, SMGASAOA of any insertion(s) in a variant gDCD in the IgVd are 20 AAs or less, 15 AAs or less, 12 AAs or less, 10 AAs or less, 7 AAs or less, or 5AAs or less in size (e.g., 1-4 AAs or 1-3 AAs). In aspects, gDP(s) lack any strand(s) that diminish binding to OSMOA gDR(s). E.g., in aspects gDP(s) include gDCDs RVRHROSI to BHV-1 gD, but which lack the nectin-1-binding diminishing G-strand/α2-helix interloop of BHV-1 gD. In aspects, such a gDP comprises a substitution of mature BHV-1 gD R188 (e.g., with Gly). In aspects, such gDP(s) exhibit at least 2×, ≥3×, or at least 4×affinity for N1. See, e.g., Yue D, et al. Science Advances. 2020: Vol. 6, no. 20.

In aspects, as described elsewhere, a CDCTFD or a gDS corresponding to a gDS in a WT gD positioned downstream of the CDCTFD comprises 1+ insertion(s). In aspects a CDCTFD or downstream gDS comprises a single insertion. In aspects, the length of such insertion(s) total 15-350 AAs, 20-300 AAs, 25-250 AAs, 30-180 AAs, 30-150 AAs, 40-240 AAs, 40-200 AAs, 50-250 AAs, or 50-200 AAs. In aspects, MGASAOA of such insertion(s) comprise Ag(s). In aspects, such Ag(s) are associated with PTPS(s), FL(s), MSL(s), SCS(s), or combinations. In aspects, AARSs inserted in the IgVD or near the IgVD (e.g., within 25 AAs, 15AAs, or 10 AAs of either or both ends) are mostly, generally, or substantially only composed of rigid residues. In aspects, insertion of residues in the C-terminal portion of a CDCTFD or downstream thereof (e.g., in a PFD or downstream of a PFD) are mostly, generally, or substantially composed of flexible residues.

In aspects gDP(s) comprise a gD profusion domain (PFD) (e.g., a domain that corresponds to a PFD of a WT a-HV, such as PRV or an HSV). In HSV-1 gD, the WT PFD is located in/about AAs 286-330. See, e.g., Cocchi F et al. PNAS. 2004; 101(19):7445-7450. In aspects, gDP(s) comprise a flexible hinge domain, which corresponds to the AARS upstream of the PFD and the N-terminal portion of the PFD in HSV-1 gD (AAs 281-292). In aspects, a portion of the PFD associates an overlapping domain, if present in the gDP, in the gDR-unbound state (similar to how AAs 293-331 of HSV-1 gD turn and partially run anti-parallel (e.g., at about AAs 311/315-331) to HSV-1 gD AAs 48-57/58). In aspects, such gDP(s) comprise a functioning PFD. In aspects, gDP(s) lack a functioning PFD. In aspects, the PFD in a gDP is proline-rich, comprising at least 5%, at least 10%, at least 15%, or at least 20% Pro residue content. In aspects, residues corresponding to most or all of HSV-1 gD P291, P292, and W319 are maintained in a functioning PFD. In aspects, AAs corresponding to MGASAOA of HSV-1 gD AAs Phe248, Asn252, Thr255, Val256, Tyr259, Ile315, Asn318, and His320 are maintained. In aspects, gDP(s) lack any sequence corresponding to the the C-terminal portion of a PFD. E.g., in aspects, no sequence corresponding to AAs 300-340, e.g., 300-335, 302-335, 302-340, 305-335, or 305-340 are contained in the sequence. In aspects in which gDP(s) lack TMD(s) and cytosolic domains such gDP(s) lack any gDS(s) that corresponding to any such downstream gDS(s).

In aspects, gDP(s) comprise an AA corresponding to W319 of HSV-1 gD that is in a similar position in the gDP with respect to a residue corresponding to F154 of an N1BD, such residues DOS promoting N1 binding. In aspects, gDPs comprise a sequence according to the formula P-X₁-X₂—W—X_{μ}—P—S—X₃-X₄—X_{Ω}-X₅-X₆—P—X₇-X₈—X₉—P-A-T-P (SEQ ID NO:720), wherein X₁ is any residue, in aspects P; X₂ is N or G; X_{μ} is 0-2 of any residues, in aspects H—I; X₃ is I or L; X₄ is Q or E; X_{Ω} is any 2 residues; X₈ is A or T; X₆ is any residue, in aspects T; X₇ is any residue, in aspects Y or P; X₈ is any residue, in aspects H or P; and X₉ is any residue, in aspects P or according to the formula P-Xα-A-P-Xp-X₂—P—X₃-X₄-W—X₅-X₆—P—X₇-XA-P (SEQ ID NO:721), wherein Xu is any ˜4 AAs, Xp is any 2-3 AAs, X₂ is I or V; X₃ is any residue, in aspects P; X₄ is N or G; X₈ is any residue, in aspects H or P; X₆ is any AA, in aspects I or Q; X₇ is S or A; and XA is ˜9 AAs comprising 1-4 P AAs. In aspects such a gD comprises GAOA of SEQ ID NO:722. Additional PMCs relating to PFD content/modification are provided in Li A et al. PLoS Pathog. 2017; 13(5): e1006314.

In aspects, gDP(s) lack a PFD. In aspects, such gDP(s) exhibit enhanced gDR binding for gDR(s) (e.g., N1, HVEM, or both). In aspects, such gDP(s) exhibit at least 5×, ≥10×, ≥15×, ≥20×, ≥35×, at least 50×, or at least 80×gDR affinity than corresponding gDP(s) comprising a PFD.

In aspects, gDP(s) comprise a PFD but lack any RBD(s). In aspects, gDP(s) comprise a PFD but lack any IgVD or any gDCD. In aspects, an immature gDP comprises a gDSS and a PFD. In aspects, such a gDP comprises 1, 2, or 2+ ETSs, e.g., a DEC-205-binding ETS.

Portions of WT gDCDs can exhibit additional functional characteristics and gDS(s) corresponding to such functional domains can be included in gDCDs or can be separately incorporated into gDP(s). In aspects, gDP(s) comprise a functional glycosylphosphotidylinositol (GPI) anchoring domain (GPIAD). In aspects, gDP(s) that comprise a GPIAD DOS associate with cell surface membranes, even when such gDP(s) lack TMD(s). In aspects, such gDP(s) are DOS more processed by ERs in COEs, e.g., in GPI-deficient cells. In aspects, such a gDS corresponds to residues 234-337 of HSV-1 gD (described in Beghdadi-Rais et al., J. Cell Sci. 105:831-40. 1993) (e.g., a gDP can comprise a variant that is RVRHROSI/SVSHSOCE to such sequence). In aspects, gDP(s) comprise a sequence corresponding to HSV-1 gD AAs 254-294 (in aspects all of 234-254), which can act as a secretion promoting domain (SPD) in suitable gDP(s) that DOS enhances secretion of the gDP from COE (e.g., gDP(s) can comprise a gDS that is RVRHROSI/SVSHSOCE to such a sequence). In aspects, glycosylation site(s) are removed from such a gDS resulting in further DOS enhanced secretion. Additional aspects relating to such gDS(s) that can be adapted to aspects are described in US20030236396.

In aspects gDP(s) comprise transmembrane domain(s) (TMD(s)), cytoplasmic domain(s) (a.k.a. an intravirion/topological domain), or both. In aspects, gDP(s) lack any TMD, topological/cytoplasmic domain, or both. E.g., in gDP(s) that are FFs or FVs of HSV-1 gD, such gDP(s) lack any gDS corresponding to HSV-1 gD AAs 331-394, 335-394, 340-394, or 344-394. In MDV gD FFs or FVs such a gDP lacks any gDS corresponding to MDV/SuAHV-1 gD AAs 358-378 or any AAR downstream of AA 358. In PRV gD FFs or FVs, such a gDP lacks any gDS corresponding to PRV gD AAs 358-378 or any residue downstream of AA 358. gDP(s) lacking a TMD can be described as “soluble” gD(s). PMCs are in Fusco D et al. PNAS. 2005; 102(26):9323-9328.

### 3. EAT-2 PPTs

In aspects, CEPs comprise Ewing's sarcoma-associated transcript 2 (“EAT-2”) PPTs. In aspects, EAT-2 PPTs are FPs. In aspects, EAT-2 PPTs are NFPs. In aspects, EAT-2 PPTS comprise ELWT EAT-2 AARSs. In aspects, EAT-2 PPTS comprise an FF of a EL WT PPT. In aspects, EAT-2 PPTs comprise a chimeric EAT-2 PPT. In aspects, EAT-2 PPTs comprise FVs of EAT-2 PPTs. CEPs can comprise any suitable number of EAT-2 PPTs of any suitable type. In aspects, EAT-2 PPTs of CEPs DOS link SLAM family ICR(s) to phospholipase Cy, calcium fluxes, Erk kinase, or combinations. In aspects, an EAT-2-containing CEP accelerates NKC-mediated IR(s), B cell mediated IR(s), NKT cell mediated IR(s), DC-mediated IR(s), T cell-mediate IR(s), macrophage IR(s), etc.

In aspects, EAT-2 PPT CEPs DOS induce polarization and exocytosis of cytotoxic granules from IC(s), such as NKCs, CD8 T-cells, or both. In aspects, EAT-2 PPT CEPs DOS induce DC maturation, monocyte phagocytosis of DCA-associated cells, or both. In aspects, EAT-2 PPT CEPs DOS reduce CRACC/SLAMF7 IR inhibition. In aspects, EAT-2 PPTs in CEPs DOS enhance one or more aspects of immunological memory, such as enhanced memory T cell population or enhanced memory T cell IR(s). In aspects, EAT-2 PPT CEPs DOS break IC tolerance characteristic(s). In aspects, EAT-2 PPT CEPs DOS modulate Th1-biasing proinflammatory cytokine and chemokine responses in TRs.

In aspects EAT-2 PPT-related IR(s) comprise DOS increases in IC expression of IL-1α, IL-2, G-CSF, IL-5, IL-12p70, GM-CSF, IL-10, TNFα, IL-12p40, IL-6, IL-9, RANTES, MCP-1, MIP-1α, MIP-10, IFN-γ, or combinations. In aspects, EAT-2 CEPs enhance IL-1β production 2×, 3×, 4×, or more; enhance IL-6 production 3×, 4×, 5×, or more; enhance TNFα2×, 3×, 5×or more; enhance G-CSF production 3×, 4×, 5×, or more; enhance IL-17 production 33%+, 50%+, or 75%+; enhance IL-10 production 1×, 1.5×, 2×, or more; etc. Aspects of WT functions of EAT-2 that can be exhibited in EP(s) are described in, references cited in US20200325182.

In aspects, an EAT-2 PPT is a human EAT-2 (SEQ ID NO:17), a murine EAT-2 PPT, or an FF or FV of either thereof. Additional EAT-2 encoding sequences and AARSs are known (see, GenBank Access Nos./NCBI protein database entries NM_012009.4, NM_012009, 148747582, NM 012009.5, 54792745, NM_053282.4 and Uniprot Entries 014796 and 035324). In aspects, an EAT-2 PPT is a canine EAT-2 or an FF or FV. In aspects, an EAT-2 PPT is a chicken EAT-2 PPT or an FF or FV thereof (See, e.g., GenBank NM_001278073.1). In aspects, an EAT-2 PPT is a bovine EAT-2 PPT or an FF or FV thereof (see GenBank NM_001193162.1).

In aspects, an EAT-2 PPT, such as an EAT-2 variant, which comprises an AARS comprising generally all, substantially all, or all of a sequence according to the formula M-D-L-P—Y—Y—H-G-Xα-L-T-K-X₁-X₂—C-E-X₃-L-L-L-K-Xa-G-V-D-G-N—F-L-X₄—R-D-S-E-S-X5-P-G-X6-L-C-L-C—V-S-F—K-X7-X8-V—Y-X9-Y—R—I-F-R-E-K—H-G-Y—Y—R—I-Q-T-Xα-Xu-Xα-X10-P-X11-Xα-X12-F—P-X13-L-X14-E-L-X15-S—K-X16-Xα-K—P-X17-Q-G-X18-V—V-H-L-Xα-X19-P-I-Xα-R-X20-X21-Xα-Xu-Xα-R-Xα-R-G-X22-X23-L-E-L-X24-X25-X26-Xα-N-X27-X28-Xα-X29-Y—V-D-V-L-P (SEQ ID NO: 723), wherein Xu represents any AA, X1 is Q or R; X2 is D or E; X3 is T or A; X4 is L or I; X5 is I or V; X6 is V or A; X7 is N or K; X8 is I or L; X9 is T or S; X10 is S or T; X11 is K or R; X12 is V or I; X13 is S or N; X14 is K or Q; X15 is I or V; X16 is F or Y; X17 is N or G; X18 is M or L; X19 is K or N; X20 is T or N; X21 is S or N; X22 is L or M; X23 is K or E; X24 is E or N; X25 is T or V; X26 is F or Y; X27 is S or T; X28 is N or D; and X29 is D or E and which suitably, comparably, or improvingly exhibits any one or more EAT-2 function(s), such as the functions of EAT-2 PPTs described above.

In cases, CEPs comprising EAT-2 PPT(s) also comprise a SLAMF CPCR PPT, e.g., a SLAMF5 CPCR PPT or a SLAMF7 CPCR PPT. In methods, a SLAMF CPCR is expressed in AAW delivery of CEPESC(s) including EAT-2-ES(s). In aspects, CEPs comprise both EAT-2 PPT(s) and SAP PPT(s). In aspects, CEPs comprise EAT-2 PPT(s), SAP PPT(s), and SLAMF6 CPCR PPT(s), which DOS enhance NKC activation. In cases, CEPs comprise activating peptidic ligand(s) for a SLAMF receptor in addition to EAT-2 PPTs. In aspects, EAT-2 AARS(s) are included in a FP, e.g., a gDFP. In cases, EAT-2 AARS(s) are included in gDAgFP(s). In cases, EAT-2 AARS(s) are expressed from different NAM(s) than the NAM(s) encoding any gDP(s) in a CEPESC.

### b. Vectors

Constructs/NS(s) can be combined with other NS(s) that, i.a., (1) allow for stable replication of the combined construct, (2) aids in transmission of the construct to a host, (3) aids in the expression of a coding sequence or an expression cassette, or (4) a combination thereof. Such larger NS constructs that facilitate reproduction, delivery, or application of EPES NS(s) are often referred to as “vectors.” Vector(s) can comprise non-coding spacer sequences (forming a backbone), and of other nucleotide sequence(s) (e.g., an origin of replication, a sequence encoding a selectable marker, site(s) for NS/construct insertion, cloning site(s), etc.). Skilled practitioners reading this disclosure will understand that is overlap between what can be classified as a “vector” and terms such as construct, expression cassette, etc., particularly in the case of non-viral DNA vectors (sometimes referred to as “DNA vectors” or “DNA vaccines”), such as plasmid vectors (sometimes “plasmids”).

Examples of suitable delivery/expression vectors include, e.g., a bacterial delivery vector, a DNA vaccine delivery vector, an RNA vaccine delivery vector, a virus delivery vector, a virus-like particle, or a composition that comprises such a vector in combination with other delivery-enhancing materials, such as a liposomal delivery vector, a transformed cell (e.g., a eukaryotic cell such as a producer cell or a DC or an attenuated bacteria) comprising a number of nucleic acid vectors, or a nucleic acid-loaded nanoparticle. A vector backbone can include sequences that sustain expression activity, such as transcription factor binding sites. In aspects, vector(s) comprise minimally sized vectors (e.g., “minicircle” vector(s)) (See, e.g., US20040214329 & US20110244566). In aspects, vector(s) comprise a mini-intronic plasmid (MIP) (see references cited in US20200325182). Further aspects relating to MIP vectors are provided in US20200325182. Vector(s) can comprise self-replicating vector(s) (see, e.g., Langle-Rouault F et al. J Virol. 1998; 72(7):6181-6185). In aspects, a vector is a self-partitioning vector (see, e.g., Piechaczek et al. Nucleic Acids Res. 1999; 27(2):426-428). In aspects, vectors do not integrate with the host genome. In aspects, compositions and methods comprise use of non-viral nucleic acid vector(s). Nucleic acid vectors (NAVs) (a) do not include ≥˜50%, and in aspects ≥˜75%, or ≥˜85% of any viral genome, (b) do not result in the expression of complete viral particles capable of invading host cells, or (c) both (a) and (b). NAVs can be RNA constructs, such as RNA vaccines described above, or DNA vectors, such as DNA plasmids or linear expression elements. Numerous plasmid backbones are available in the art, including pVax and pcDNA3 plasmids (Thermo Fischer, Scientific, Waltham, MA, USA). Linear vector constructs are described in references cited in US20200325182. NAVs are further reviewed in, e.g., Rodriguez EG. Nonviral DNA vectors for immunization and therapy: design and methods for their obtention. J Mol Med (Berl). 2004; 82(8):500-509 and G6mez and Onate, “Plasmid DNA Vaccines” DOI: 10.5772/intechopen.76754. An exemplary plasmid vector used in aspects is provided in the Examples, below (plasmid constructs pMBF116-CMVp-Ub-CP204L-T2A-GFP-TcnR & plasmid pMBF117-CAGp-Ub-CP204L-T2A-GFP-TcnR). DNA constructs useful in the present compositions can be “naked” DNA vectors (as described in, e.g., Restifo et al. Gene Therapy 2000; 7:89-92). Naked NAVs are compositions of nucleic acids that are free of any association with viral capsid proteins in stable composition or when delivered in a method. In aspects, nucleic acid vectors used in compositions or methods of the invention are not associated with any polypeptides, in particular not with polypeptides of viral origin, when in stable composition or administered to a subject (however, such constructs may encode one or more viral polypeptides as antigens). NAVs also can be associated with various TFAs. Examples of known NAVs suitable for aspects include minicircle, minivector, miniknot, MIDGE vectors (Schakowski et al. (2001) Mol Ther 3:793-800), MiLV, Ministring, and Mini-intronic plasmid vectors. Examples of vectors also are described in Hardee C L, et al. Genes (Base1). 2017; 8(2):65 & Williams JA. Vaccines (Base1). 2013; 1(3):225-249.

A vector that is either composed of or encodes a viable virus capable of infecting host cells is referred to herein as a “viral vector.” In aspects, compositions comprise, or methods comprise use of, viral vector(s). In an exemplary embodiment, a viral vector is selected from the group consisting of adenovirus vectors, adeno-associated virus (AAV) vectors (e.g., AAV type 5 and type 2), alphavirus vectors (e.g., Venezuelan equine encephalitis virus (VEE), sindbis virus (SIN), semliki forest virus (SFV), and VEE-SIN chimeras), flaviviral vectors, herpes virus vectors (e.g. vectors derived from cytomegaloviruses, like rhesus cytomegalovirus (RhCMV), arena virus vectors (e.g. lymphocytic choriomeningitis virus (LCMV) vectors), measles virus vectors, papillomaviral vectors, and poxvirus vectors. In aspects, viral vector(s) are extrachromosomal vectors. In aspects, vector(s) are replication-deficient vector(s). Specific examples and extensive disclosure of vectors is provided in US20200325182 and references cited therein. In aspects, vector(s) comprise adenoviral (Ad) vectors, e.g., derived from a human adenovirus vector, a simian adenovirus, etc., or comprising a group B Ad vector, a group C Advector, a group E adenovirus vector, an adenovirus 6 vector, a PanAd3 vector, an adenovirus C₃ vector, a ChAdY25 vector, an AdC68 vector, or an Ad5 vector.

In aspect(s), NAM(s) is/are incorporated in or delivered by a cellular delivery system, e.g., an attenuated “bacterial vector,” e.g., an attenuated Salmonella typhimurium, Salmonella typhi, Shigella, Bacillus, Lactobacillus, Bacille Calmette-Guerin (BCG), E. coli, Vibrio cholerae, Campylobacter, Listeria, etc. Other cellular systems (e.g., DCs) are described elsewhere. Bacterial vectors typically are attenuated/non-pathogenic vectors. See US20200325182. In cases, a bacterial vector DOS promotes delivery of NAM(s) and increases expression levels in context(s), e.g., in delivery of CEPESCs in, i.a.,/e.g., gastrointestinal (GI) contexts. Examples of such vectors are provided in, e.g., Song Y. et al. J Vet Med Sci. 2020 Dec. 5; 82(11):1693-1699 and Zhi. Vaccines 2021, 9, 22 (both regarding attenuated Salmonella vectors) and Kong. (2012). PNAS November 20; 109(47):19414-9 (regarding Bacillus vectors).

In aspects, vector(s) generally are non-infectious, non-replicating, nonintegrating, non-pathogenic (e.g., having MGASAOA ES(s) of pathogenic PPTs deleted/modified), or a combination thereof. Aspects of such vectors are described in US20200325182 and references cited therein.

In aspects, vectors comprise NS(s) of at least about 2.25 kb, such as ≥2.5 kb, ≥3 kb, or ≥4 kb (e.g., about 2⁻⁵ kb, ˜2.1-4.9 kb, ˜2.2-4.8 kb, ˜2.25-4.75 kb, ˜2-4 kb, ˜2-3 kb, about 2.1-3.1 kb, ˜2.3-3.3 kb, ˜2.3-3.8 kb, or about 2.3-about 2.5 kb). In other aspects, vector(s) comprise ≤1.5 kb, less than about 1.25 kb, ≤˜1 kb, ≤˜ 0.85 kb, ≤˜0.75 kb, ≤˜0.5 kb, ≤˜0.33 kb, or ≤˜0.25 kb outside of the expression cassette(s) in the vector.

In aspects, NAM(s) or vector(s) express multiple separate EP(s). E.g., vector(s) can comprise ≥2 NS(s) expressed in a host cell as separate transcripts (e.g., ≥2 expression cassettes), generate ≥2 cistrons (e.g., ≥1 expression cassettes comprising coding sequences that are processed to form two or more discrete polypeptides when the coding sequences are expressed). Vectors comprising ≥2 separately expressed EPES(s) are aka “multi-gene vectors.” A vector comprising two expression cassettes may be described as a “bicistronic” vector (and a vector comprising 3 separately expressed EPES(s) can be called a “tricistronic” vector). Multicistronic expression can be achieved through IRES(s) furin cleavage sites, viral 2A peptide sequences, etc., discussed elsewhere (see also, e.g., Shaimardanova et al., Pharmaceutics 2019, 11(11), 580 and US20200325182). In aspects, a vector also can comprise multiple expression cassettes, multiple promoters, or promoters with certain capabilities (such as bi-directional promoters) that drive expression of different transcription units. PMCs are provided in Volker Thiel et al., Journal of Virology August 2003, 77 (18) 9790-9798; Zhu Y et al. Mol Ther. 2001; 4(4):375-382; and Kriz A et al. Nat Commun. 2010; 1:120. In aspects, the vector or construct also obtains multiple expression products through incorporation of a bidirectional promoter. See, e.g., Javan et al. Life Sci. 2018; 202:140-151; Léjard et al. Plasmid. 2014; 74:1-8; Polson et al. Plasmid. 2011; 66(3):169-179. Also still, splicing signals can be incorporated into a construct to generate several mature mRNAs from a single pre-mRNA expressed from an expression cassette. An example of such a splicing strategy for obtaining a multi-cistronic vector is described in, e.g., Zhu Y et al. Mol Ther. 2001; 4(4):375-382.

In aspects, vector(s) in CEPESCs are targeted vectors. Targeted vector(s) comprise feature(s), that direct the vector to particular target(s), e.g., cell receptor(s), in TRs. In aspects, targeted vector(s) target ICR(s), such as ITIC receptor(s) (e.g., a DC receptor). In aspects, targeted vector(s) are viral vectors. Viral vectors, such as Vaccinia Virus (VV), Modified virus Ankara (MVA), certain lentiviral vectors, retroviral vectors, and Ad vectors exhibit targeting of DCs and other ICs. Viral vectors also can be modified to include heterologous PPTs that specifically bind targets, such as Ab AARSs against ICRs. In aspects, targeted vectors are non-viral vectors, such as NAVs. Targeting compound-conjugated NAVs such as NAVs conjugated to PPTs/moieties targeting ICs, e.g., DCs, also are known in the art (See, e.g., Anderson K et al. Bioconjug Chem. 2010; 21(8):1479-1485). Vectors also can be targeted to internal targets. E.g., plasmid vectors can be conjugated to nuclear localization signals (NLSs) (e.g., Arg/Lys rich AARSs recognized by karyopherins such as importin a resulting in facilitated transport across the nuclear envelope) (See, e.g., Hardee C L, et al. Genes (Base1). 2017; 8(2):65).

c. Compositions Comprising NAMs/Vectors and Components Thereof

Compositions/CEPESC(s) can comprise 1+, 2+, 3+, or more NAMs (e.g., NAVs or other vectors) that collectively comprise the CESs of the composition (i.e., contain all of the EPES(s)/CS(s), encoding all of the EPs of the composition). In aspects, OSMGAOA of the NAMs, NAM molecules, or both are associated with one or more delivery agent(s) (DA(s)). In aspects, CEPESC(s) comprise one or more excipient(s). In aspects, CEPESCs comprise both. CEPESCs are suitable for pharmaceutical use, veterinary use, etc. Such compositions can be described as “pharmaceutical” or “veterinary” or suitable. E.g., compositions can comprise 2+ vectors, (e.g., 2, 2-3, or 2⁻⁵ vectors); 2+ EPESNAM(s); or both. In aspects, each EPESNAM is contained in a separate vector in a composition. Thus, for example, compositions of the invention can comprise, e.g., 1⁻⁵ vectors, 1-4 vectors, or 1-3 vectors. One such exemplary composition/method comprises one vector that comprises a gD-antigen fusion protein-encoding sequence and a second vector that comprises a non-antigen immunomodulator, such as an innate trained immunity immunomodulator (ITII), such as an EAT-2 polypeptide, or a cytokine. In another exemplary aspect, which can be reflected in a composition of the invention or method of the invention, at least a first vector, such as a plasmid vector, comprising a gD-antigen fusion protein-encoding sequence is combined with or used with a second plasmid vector comprising a different antigen-encoding sequence, which optionally may be associated with a targeting sequence/domain, a different innate cell activator sequence or polypeptide, or both.

In aspects, compositions/CEPESCs comprise delivery agent(s) associated with NAM(s). Combinations of NAM(s) and delivery agent(s) can be referred to as “delivery systems.” A delivery agent (“DA”) is any composition that DOS enhances the uptake of the NAM(s) resulting in expression of the ES(s) they contain. DAs also are referred to herein as transfection-facilitating agents or TFAs. Some aspects of DAs and other components of CEPESCs, such as PPT components of viral vectors, can overlap. Skilled readers will understand that such categorization(s) of CEPESC components are presented in a non-limiting manner as a matter of convenience in describing aspects.

NAM(s) can be bound to, coated on, or contained in particle DAs (PDAs). In aspects, NDA(s) are coated with additional DA(s), e.g., cationic composition(s), targeting compositions (e.g., ligands for ICR(s)), or both. In aspects, NAMs are in viral vectors comprising viral nanoparticles or viral-derived nanoparticles. CEPESCs also can comprise virus-like particle PDA(s).

As with NAMs, terms such as “particles” and “nanoparticles” typically refer to types of particles/nanoparticles and not specific particle molecules, which can, e.g., number in the millions in CEPESCs.

In aspects, TFA(s) are targeted to tissue(s), organ(s), or cell type(s). As also described elsewhere, targeting method include incorporation of Ab AARS(s), ligand AARS(s), or other compounds, e.g., aptamers to ICR(s) or CpG(s) that are bound by DEC-205, etc., which are either bound directly to NAM(s) or are bound or otherwise associated with PDA(s) (e.g., a liposome PDA, a polymeric DA, or protein component(s) of a viral vector). Such and other PDA(s) can comprise, e.g., DNA binding proteins or polypeptides, liposomes, extracellular vesicles, exosomes, or other positively charged macromolecules used alone or in combination) that binds NAM(s) of the CEPESC. Extracellular vesicles are membranous vesicles released by a variety of cells into the extracellular microenvironment and include ectosomes or microvesicles (ii), exosomes and (iii), apoptotic bodies. Such materials are described in references included in US20200325182. Transfection facilitating agent(s) (TFA(s)) also can include surface active agents/surfactants, such as immune-stimulating complexes (ISCOMS), LPS analog(s) including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known TFA(s). Polyanion and polycation TFAs are known, including poly-L-glutamate (LGS). In aspects, TFA(s) comprise poly-L-glutamate, lecithin liposomes or other liposomes known in the art (See, e.g., WO9324640), or calcium ions. In aspects, NAM(s) are CB DA/PDA full encapsulation, partial encapsulation, non-encapsulation (conjugation), or a suitable combination thereof. Numerous specific DAs, such as polyethylene glycol (PEG)-associated DAs, amphipathic lipid Das, phospholipid DAs, neutral/non-cationic/cationic lipid DAs, etc., are provided in US20200325182. CEPESCs can comprise any suitable number of DAs of any suitable composition. In aspects, CEPESCs comprise 2+, 3+, or more DAs associated with SMGAOA of the NAMs of a CEPESC. In aspects, CEPESCs comprise a single type of DA, which can comprise several components of different origins/compositions or a single type of origin. In aspects, CEPESCs comprise solid nanoparticle(s). In aspects, CEPESCs comprise liposome DA(s), or both. In aspects, CEPESCs comprise VLP DAs. In aspects, CEPESCs comprise polymeric DAs (e.g., Poly(lactide-co-glycolide) (PLGA), Poly(lactic acid) (PLA), or chitosan polymers). In aspects, DAs comprise inorganic nanoparticles (e.g., gold nanoparticles silica nanoparticles, or CaPNP(s). In aspects, DAs PC, GCO, SCO or CO of CaPNP(s). DAs also can comprise, primarily comprise, GCO, or CO dendrimers, small unilamellar vesicles (SUVs) or large unilamellar vesicles (LUVs), collagen, chitosan, hyaluronic acid, etc. See US20200325182.

PDAs can comprise calcium compounds, such as for example calcium sulfates, or calcium phosphates (e.g., calcium phosphate, such as for example amorphous calcium phosphate, as primary particle materials, coating agents, or both. In aspects, DAs of CEPESCs PCGCOSCO or CO calcium phosphate particles having greater than 75% amorphous content, e.g., greater than about 80%, ≥˜85%, ≥˜90%, or even greater, e.g., ≥˜95% amorphous content), crystalline or poorly crystalline apatitic calcium phosphate (e.g., a calcium phosphate comprising a synthetic material not necessarily restricted to 1 calcium phosphate phase), dicalcium phosphate dihydrate, tricalcium phosphate, tetracalcium phosphate, monetite, monocalcium phosphate monohydrate, octacalcium phosphates, hydroxyapatites, or carbonated or otherwise substituted/modified versions of such calcium phosphates). Aspects relating to calcium-based DAs adaptable to CEPESCs are described in US20200325182 and references cited therein (included references).

In aspects, CEPESCs comprise CaPNP nanoparticles. In aspects, the only DAs/TFAs in the CEPESC are the CaPNPs or materials associated directly with the CaPNPs (e.g., functionalizing agents). In aspects, CEPESCs comprise functionalized CaPNP(s). Functionalizing agents associated with CaPNP(s) comprise lipids, polycations, polyanions, citrates, etc. (e.g., polyethyleneimine, poly-lysine, etc.) (see, e.g., U.S. Pat. No. 8,309,134).

In aspects, DAs/TFAs are biodegradable, resorbable, or both. Examples of such particles and other DAs/TFAs are in US20200325182.

In aspects, TFA(s) comprises calcium phosphate nanoparticles (CaPNPs). In aspects, CaPNP(s) are multi-layer CaPNP(s). In aspects, the composition comprises a multi-layer CaPNP structure comprising a CaPNP core and at least one CaPNP outer layer that encompasses NAM(s).

In aspects, most, generally all, substantially all, or all of the DAs/PDAs of a CEPESC are sized such that the composition and accompanying NAM(s) can be taken up by ICs, e.g., macrophages, DCs, NKCs, T-cells, or combination(s). Also, a delivery system can have an average size or a maximum size such that the CEPESC can be delivered by an intramuscular injection using standard sized injection needle(s).

In aspects, CEPESC(s) comprise DA(s) less than 2 microns in size. In aspects, CEPESC(s) comprise, primarily comprise, generally comprise, or only comprise nanoparticle-sized DA(s). Nanoparticle DA(s) are made of pharmaceutically suitable materials that enhance cellular uptake of NAM(s) and that on average, in maximum diameter, or both, are less than 1 micron in size (or that are generally, substantially only, or only less than 1 microns in size). In aspects PDAs PCGCOSCO or CO PDAs of less than 0.7 microns, less than 0.5 microns, or less than 0.4 microns in size. In aspects, PDAs are on average, generally, substantially only, or only, less than 0.35 microns or 0.25 microns in size (e.g., 5-350 nm, such as 50-300 nm, e.g., 100-250 nm, 150-225 nm, or 180-215 nm). In aspects, DAs, such as PDAs are less than about 0.15 microns, ≤˜0.1 microns, ≤˜0.05 microns, less than 0.025 microns, or less than 0.01 microns in average size or maximum size. According to embodiments, nanoparticles of a composition have an average/maximum diameter of less than 300 nm (“diameter” as used herein being used to describe the largest dimension of a nanoparticle used for or used as part of a delivery mechanism even if the nanoparticle is not exactly spherical), such as for example ≤˜250 nm in diameter, ≤˜200 nm, ≤˜150 nm ≤˜100 nm, ≤˜50 nm, or ≤˜ 25 nm in diameter, e.g., ˜10⁻²⁰⁰ nm, ˜25 nm-175 nm, ˜50 nm-150 nm, ˜75 nm-125 nm. In aspects, a nanoparticle has an average size of less than 10 nm, such as about 10 nm, about 8 nm, about 6 nm, about 4 nm, or about 2 nm. In aspects, the nanoparticle is sized to be capable of being DOS phagocytized by ICs, such as ITIC(s), e.g., macrophage(s), in TR(s).

In aspects, NAM(s) is/are conjugated to PDA(s). In aspects, NAM(s) is/are bound to most, generally all, or all of the surface of such PDA(s) or areas intended to be associated with NAM(s) (NAM-associated area(s) that make up less than all of the PDA. In aspects, PDA(s) comprise a layered design. See, e.g., US20200325182. PDA(s) can have any suitable shape(s). In aspects, PDA(s) are generally spherical. In aspects, SMGAOA PDA(s) are non-spherical shape, e.g., disc-like (e.g. somewhat circular and flat) or needle-like shape. In aspects, PDAs have a smooth surface or a rough surface. In aspects, PDA(s) have a rough surface that DOS induces IR(s). In aspects, PDAs are porous. In aspects, porous PDA(s) have a specific minimum, average, or maximum porosity. In aspects, the porosity of PDA(s) DOS enhances resorption of the PDA(s) (see, e.g., WO20000015194). In aspects, PDAs are substantially or completely bioabsorbed to nondetectable levels within 2 months, within 1 month, or less, e.g., 1-24 days, or 1-14 days, 1-10 days, or 1-8 days.

In aspects, PDA(s) DOS induce uptake of associated NAM(s) into cells (e.g., target cell(s)), release of NAM(s) into the cytoplasm, uptake of NAM(s) into the nucleus, protection/stability of the NAM(s), expression of the NAM(s), and induction of IR(s). In cases, CEPESCs do not DOS induce formation of granuloma(s). impair quality of tissue (e.g., muscle), or both. In aspects, CEPESCs are DOS transfected in APCs. In aspects, CEPESC(s) are shelf-stable for periods under typical storage conditions, e.g., at FDA standard RT/ambient temperatures (e.g., 10⁻⁵⁰ or 10⁻⁴⁰ degrees C. & 40-80% relative humidity). In cases, CEPESCs do not require cold chain storage over periods of time (1 day, 1 week, 1 month, etc.) to be/remain effective.

In aspects, PDA(s) comprise material(s) or composition(s) that effectively target particular cells, such as IC(s), e.g., ITIC(s), e.g., DCs. See, e.g., Cruz U et al. J Control Release. 2014; 192:209-218; US 20130142864, WO2005018610, and Saluja S S et al. Int J Nanomedicine. 2014; 9:5231-5246. In aspects, DAs/PDAs have adjuvant properties (DOS enhancing IR(s) induced by antigen(s), inducing non-specific IR(s), or both). E.g., in aspects, PDAs comprise, generally consist of, or consist of calcium phosphate nanoparticles that independently DOS induce IR(s).

PCA(s) and NAM(s) of CEPESCs can be present in any suitable concentration/relationship. E.g., density of PDAs, such as CaPNPs is, in aspects, between ˜2 g/cm³ and ˜4 g/cm³, e.g., about 2.2, 2.6, 2.8, 3, 3.25, 3.5, or 3.6 and 3.8, 4, 4.25, 4.5, or 5 g/cm³. In aspects, most, generally all, or substantially all PDAs in a formulation do not DOS agglomerate. In aspects, about 50% or more (e.g., 60%, 75%, 90% or more) of PDAs remain suspended after 20 hours, 40 hours, 72 hours, 96 hours, or 120 hours or more. In aspects, NAM(s) are present in a concentration about 1 μg to about 10 mg (e.g., about 5 μg to about 10 mg, about 5 μg to about 5 mg, about 1 mg to about 5 mg, about 1 μg to about 2 mg, about 1 μg to about 1 mg, about 1 μg to about 500 μg, ˜1 μg to ˜100 μg, about 1 μg to about 50 μg, or ˜1 μg to ˜10 μg, e.g., ˜5-100 μg, e.g., ˜5-75, 2-50, 2-40, or 2.5-25 μg. In aspects, NAM(s) is/are present in a volume of about 0.25 mL, about 0.5 mL, 0.75 mL, about 1 mL, or about 2 ML (e.g., 0.1-3, 0.2-2.6, 0.25-2.5 mL). In aspects, viral vectors comprise at least about 1×10³ viral vector particles in a volume of about 1 mL (e.g., ≥1×10³ to about 1×10⁸ particles in about 1 mL). In aspects, viral vectors are present in an amount of ≥1×10⁶, about 1×10⁷, about 1×10⁸, about 1×10⁹, or more particles/mL). In aspects, CEPESCs comprise NAM(s) and PDA(s) in NAM/PDA ratio of about 1:1 to about 1:40, e.g., about 1:2 to about 1:20, e.g., about 1:3 to about 1:12, about 1:2 to about 1:12, about 1:3 to about 1:9, about 1:2 to about 1:8, about 1:3 to about 1:6, about 1:1 to ˜1:5, about 1:2 to ˜1:5, or about 1:3 to about 1:5. E.g., CEPESCs can comprise about 0.5-500 micrograms NAM and about 0.5-10,000 micrograms PDA, such as about 1-5,000 micrograms PDA, e.g., about 1.5-3000 micrograms PDA, about 1.75-3000 micrograms PDA, or about 2-2000 micrograms (mcg/pg) PDA, e.g., CaPNP.

PDAs such as calcium phosphate nanoparticles (CaPNP(s)) can be functionalized with substances including lipids, polycations, polyanions, or citrate. In certain aspects, a functionalized nanoparticle can be charged. In aspects, PDAs can incorporate resorption factor(s) DOS impacting the rate at which the nanoparticles are absorbed. PDAs can include targeting/attraction factors, e.g., interleukin-1, lymphotoxin, or calcitonin. PDAs can be further functionalized with anions, cations, and polymers. See US20200325182.

PDAs/CEPESCs can have any suitable charge characteristics. In aspects, a PDA(s)/CEPESCs comprise a zeta potential of between about −50 to about 50 millivolts, e.g., about −70 to about 70 millivolts, e.g., about −70-20 millivolts, or ˜−70-0 millivolts, 0-70 millivolts, ˜30 to ˜30 millivolts, etc.

Compositions can contain excipient(s) in addition to NAM(s) and any optionally present DA(s)/TFA(s). “Excipients” here means any intended, pharmaceutically acceptable component of a CEPESC that is not (1) any NAM(s) comprising EPES(s), (2) another active pharmaceutical ingredient (API); (3) a vector or vector component; (4) a DA/TFA; and (5) an adjuvant (a substance that induces non-Ag specific IR(s) or generally enhances IR(s)). Excipients can either be classified as functional excipients, which impart/exert detectable and specific functions to/on the CEPESC (e.g., preservative, stabilizer, antioxidant, surfactant, chelating agent, isotonicifier, anesthetic, or buffer) and nonfunctional excipients, that are inert carriers or bulking agents (e.g., materials classified as diluents, carriers, and the like, such as water, solutions, etc.). In aspects, CEPESC(s) comprise excipient(s), PDA(s), or both that provide modified release characteristics, such as delayed release, sustained release, or both. In aspects, CEPESC(s) lack any delayed release characteristic(s)/component(s). NAM(s) of CEPESC(s) typically are isolated, except for any intended conjugated or otherwise associated PDA(s), prior to formulation. Compositions can include carrier(s) for delivery, e.g., injection, such as water for injection (WFI), PBS, aqueous dextrose solutions, glycerol solutions, and other suitable sterile solution(s). Excipients can provide pH adjusting and isotonicity/buffering characteristics. Compositions typically are sterile, pyrogen free, and free of undesirable particulates/impurities (e.g., comprise less than 2.5, ≤1, ≤0.5, or ≤˜0.1% thereof). Compositions typically are non-toxic, non-carcinogenic, non-teratogenic, non-genotoxic, or non-immunogenic and non-antigenic (except for components intended to induce IR(s)). Aspects of excipients, compositions, etc., are found in US20200325182.

Kits represent another aspect. “Kits” can comprise any suitable combination of CEPESC components, such as NAM(s), separate NAM compositions for combination, PDA(s) that can be mixed with/reacted with NAM(s), excipients, CCC(s), or combinations. Kits can include instructions, labeling, etc. Kits can also include indicators of purity, stability, etc., and assay(s) for demonstrating potency, purity, or effectiveness (e.g., ELISA components, instructions for performing FACS, qPCR components, etc.). Kits can include indicator(s) of identity, such as RFID tags, and the like. Kits also can include containers for storing kit component(s), mixtures made in processing/making CEPESC(s), and the like. Components can be in dried or liquid form (e.g., NAM(s) can be present in lyophilized, spray dried, or other dried forms, which are an aspects). Containers can include sealed containers such as ampule or sachet indicating the quantity of component(s) (e.g., water for injection, buffers, and the like). In aspects, kit(s) or packaged composition(s) comprise two or more different types of NAM(s) or two or more different types of CEPESC(s) (e.g., a NAV CEPESC and a viral vector CEPESC or a CEPESC intended for priming IR(s) and a second CEPESC intended for boosting IR(s)). Kit(s) and packaged composition(s) can comprise single-use component(s), reusable component(s), or both. Kit(s) can include and packaged composition(s) can include delivery system component(s)/device(s), e.g., bottles, test tubes, vials, iv bags, pierceable vials, syringes, needles, dispensing pens, biolistic system(s), and the like, or systems for promoting delivery such as for performing electrophoresis. Kits and packaged compositions(s) can include materials used in administration such as alcohol or other wipes, bandages, labels, anesthetics, and the like. Additional aspects of kits are provided in US20200325182.

Cells comprising EPES/CEPES NAM(s) and methods of producing NAM(s)/constructs/vectors are additional aspects. Host cells (e.g., HeLa, BHK, MDCK, HEK 293, K8 cells, SW-13 cells, MCF7 cells, CV1/EBNA cells, PERC6 cells, NTH-3T3 cells, MRC-5 fibroblast, & WI38 cells), recombinant animals, and recombinant plants, comprising NS(s) are described in US20200325182.

PPTs expressed from CEPESCs, compositions comprising such PPTs, and the use of such PPTs or compositions, also reflect aspects. In aspects, a CEPESC EP PPT is further derivatized prior to use. A PPT “derivative,” which will be understood as a biomolecule modified by chemical modification, such as replacement with a synthetic amino acid or conjugated amino acid, as is described above. Derivatives are described in US20200325182.

In aspects, CEPESCs comprise additional API(s), adjuvant(s), or both. Such CEPESCs can be characterized as combination compositions (CCs) and such additional components can be characterized as CC components (CCCs). In aspects, a composition is intended for prevention or treatment of cancer and CCCs comprise anti-cancer therapeutics, anti-cancer prophylactic agents/vaccines, or both. In aspects, a composition is intended for prevention or treatment of a pathogenic DCA, such as a virus. Numerous examples of each are described in, e.g., US20200325182. CCCs can include ICs (e.g., DCs or other APCs (including treated cells such as pulsed DCs), autologous ICs/cells, killed cells, sera, or modified/synthetic cells, such as CAR-T cells). In aspects, CCCs comprise CIs, e.g., an anti-PD-1, anti-CTLA4, anti-KIR, anti-CRACC, or anti-PD-L1 Ab. In aspects, anti-cancer compositions include chemotherapeutic agents. In aspects, CCCs include cytokine(s) or NAMs that express cytokines (this principle is applicable to other aspects). CCCs also can include immunomodulators (including non-peptidic IMs such as imiquimod and resiquimod or CpG oligo- and di-nucleotides).

In aspects, CCCs or AACs comprise one or more vaccines, e.g., PPT vaccines, inactivated virus vaccines, DNA vaccines, RNA vaccines, etc. In aspects, CCCs induce a predominantly or generally only Th1-type response.

In aspects, CCCs/AACs are adjuvant(s). Adjuvant(s) induce non-Ag-specific IR(s) or enhance other IR(s) in TR(s). Adjuvants may be selected from any of the classes (1) mineral salts, e.g., aluminum hydroxide and aluminum or calcium phosphate gels; (2) emulsions including: oil emulsions and surfactant based formulations, e.g., microfluidised detergent stabilised oil-in-water emulsion, purified saponin, oil-in-water emulsion, stabilised water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensoactive compounds; (9) carbohydrates; or combinations thereof. Cytokine(s) can exhibit adjuvant activity and be considered adjuvants. Examples of adjuvants and other CCCs/AACs are provided in US20200325182.

II. Methods of Inducing Immune Response(s)

Methods of using CEPESCs and other compositions described here to induce IR(s) or CE(s) and, in aspects, prevent or treat diseases associated with DCAs in TR(s), e.g., pathogen infections or cancers, are embodiments. Methods can be applied in any suitable TR, including human patients and non-human subjects, such as swine, cows, dogs, cats, chickens, horses, and the like (e.g., companion animals and livestock animals). In aspects, CEPESCs are used as therapeutic or prophylactic agents. In aspects, CEPESCs are delivered one or more times to TR(s). CEPESCs can be delivered/administered to TRs by any suitable technique(s), into any suitable tissue(s), any suitable number of time(s), and in any suitable amount(s). In aspects, CEPESCs are delivered in a single dose, in several divided dosages, or staggered dosages. In aspects, CEPESCs are administered daily or sequentially. In aspects, CEPESCs are continuously infused. In aspects, CEPESCs are delivered via bolus injection. Different CEPESCs, CCCs, dosage amounts, number of dosages, etc., can be applied also based on CEs, AEs (e.g., cytokine syndrome effects), etc. In aspects, CEPESC(s) are used to stimulate cells subsequently infused into TR(s).

In aspects, CEPESCs are delivered to TRs by intramuscular, subcutaneous, intradermal, intravenous, inhalation, insufflation, oral, nasal, rectal, parenteral, sublingual, paracanceral, transdermal, transmucosal (e.g., (trans)buccal, (intra)nasal, and (trans)rectal), intravesical, intrapulmonary, intraventricular, intraduodenal, intra-tumoral, intragastrical, intrathecal, intra-arterial, or intrabronchial administration. In aspects, CEPESCs are delivered by mucosal administration. In aspects, CEPESCs are delivered by injection (e.g., i.m. or s.c. injection). In aspects, CEPESCs are delivered intradermally, e.g., by biolostic/gene gun delivery methods (SFE Haynes et al, J Biotechnology 44: 37-42 (1996) & U.S. Pat. Nos. 5,846,796; 6,010,478; 5,865,796; & 5,584,807).

In aspects, delivery of CEPESCs into target cell(s) is facilitated by application of electroporation. Other methods applicable to delivery of CEPESCs comprise protoplast fusion, cationic liposome-mediated transfection; tungsten particle-facilitated microparticle bombardment; and strontium phosphate DNA co-precipitation. See US20200325182. In embodiments, compositions are administered to muscle, either skeletal muscle or cardiac muscle, or to lung tissue. Such methods are exemplified in, e.g., Wheeler, C. J., et al., Proc. Natl. Acad. Sci. USA 93:11454-11459 (1996). In cases, compositions are delivered through intramuscular (i.m.), intradermal (i.d.), subcutaneous (s.c.), or intrapulmonary routes. Other suitable routes include intratracheal, transdermal, intraocular, intranasal, inhalation, intracavity, intravenous (i.v.), intraductal (e.g., into the pancreas) and intraparenchymal (i.e., into any tissue) administration. In aspects, CEPESCs are delivered via transdermal (e.g., percutaneous) and transmucosal administration (i.e., into or through skin or mucosal tissue). In aspects, CEPESCs are delivered by ≥2 methods (e.g., by two of i.m., s.c., and mucosal delivery routes). Delivery of NAMs to interstitial spaces of tissues of an individual is described in, e.g., US20200325182. Administration means for CEPESCs can include needle injection, catheter infusion, biolistic injectors, etc. In aspects, CEPESCs can be delivered by transdermal administration (e.g., aided by iontophoresis, electroporation, or use of a PDA, such as a tat-conjugated dendrimer). Epidermal administration can also be employed in some cases. In aspects, CEPESCs are formulated for administration to mucosa, e.g., via the nasal passages.

In aspects, methods comprise administering a CEPESC to a TR one single time. In aspects, a CEPESC is administered to a TR at two different times or 2 different CEPESCs are administered on different times. In aspects, CEPESC(s) are administered three times (e.g., a single CEPESC is administered at 3 different times, 3 different CEPESCs are administered at different times, or methods comprise some mixture of repeat administration and administration of different CEPESC(s)). In aspects, CEPESCs are administered four times to TR(s). In aspects, CEPESC(s) are administered more than four times to TR(s). In aspects in which multiple administrations of CEPESC(s) are performed, such CEPESC(s) can be administered at regular intervals, e.g., daily, weekly, every two weeks, every three weeks, every month, every 2 months, every quarter, semi-annually, annually, etc. Each possibility represents a separate embodiment of the methods disclosed herein. To exemplify, a dose of CEPESCs can be delivered to TR(s) every 1-2 weeks, every 2-3 weeks, every 3-4 weeks, every 4⁻⁵ weeks, every 6-7 weeks, every 7-8 weeks, every 9-10 weeks, or every 1-24, 1-18, 1-12, 1-9, 1-6, 1-4, 3-4, 4-5, 6-7, 7-8, 9-10, 1-3, 2-3, or 1-2 months in order to achieve the intended elicitation of IR(s)/CE(s). In aspects, repeat administrations (booster doses) of CEPESCs are applied to TR(s) immediately following the first course of treatment or after an interval of days, weeks or months to induce intended IR(s)/CE(s). In one embodiment, a subject is administered a booster dose every 1-2 weeks, every 2-3 weeks, every 3-4 weeks, every 4⁻⁵ weeks, every 6-7 weeks, every 7-8 weeks, or every 9-10 weeks in order to achieve the intended anti-tumor response. In one embodiment, a subject is administered a booster dose every 1-2 months, every 2-3 months, every 3-4 months, every 4⁻⁵ months, every 6-7 months, every 7-8 months, or every 9-10 months in order to achieve the intended elicitation of an immune response targeted at the subject's disease or condition. E.g., CEPESCs can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1-10, 2-10, 1-5, 2-4, 1-4, 1-3, or 2-3 times over a period of 2 years, 1.5 years, 12 months, 9 months, 6 months, or 3 months (such administrations separated by 0.5-24 months, e.g., 1-12 months, 1-6 months, or different periods falling within any such range). The number of total applications of CEPESC(s) can be, e.g., 1, 2, 3, 4, 5, 6, 7, 8, 9, or 10 times. In aspects, delivery of multiple CEPESCs; multiple doses of CEPESCs, ≥2 CEPESC(s)+ CCC(s); or application of AAC(s) and delivery of CESPESC(s) comprises step(s) of monitoring efficacy (e.g., against standard(s)), monitoring AE(s) (e.g., cytokine syndrome, immunogenic reactions, and the like), or both, and adjusting the timing of delivery, amount of delivery, frequency of delivery, dosage amount(s), or selecting second or more subsequent CCC/CEPESC to deliver or AAC to apply. E.g., In aspects, methods comprise performing a method that DOS stimulates DCs, NKCs, or both, optionally with other ITICs or ICs in a population of TRs (e.g., as determined by clinical study or studies) in a TR and thereafter delivering CEPESC(s) to the TR. In one case, a composition comprising an effective amount of an EAT-2 PPT or EAT-2-ES NAM is delivered to a TR and after either a period of time that normally is sufficient for IC stimulation or after the detection of stimulation in the TR CEPESC(s) are administered (e.g., a CEPESC comprising gDAgFP(s)). CEPESCs can be delivered in any suitable dosage(s) delivered any suitable number of time(s). In aspects, each dosage of CEPESC/NAM(s) ranges from ˜0.001 to 30 mg/kg TR body wt, ˜0.01 to 25 mg/kg body weight, about 0.1 to 20 mg/kg body weight, or from about 1 to 10 mg/kg, 2 to 9 mg/kg, 3 to 8 mg/kg, 4-7 mg/kg, or 5-6 mg/kg body wt.

In multiple CEPESC methods or combination methods, CEPESCs or CEPESC(s) & CCC(s)/AAC(s) can be administered simultaneously, for example in a combined unit dose or separately in a specified time interval, e.g., in an interval of minutes, hours, days or weeks. Such agents may be administered in any order, or as 1+ preparations that includes 2+ agents. In aspects, at least one administration of one of the agents, e.g., a “first agent,” may be made within minutes, one, two, three, or four hours, or even within one or two days of the other agent, e.g., a “second agent.” In some embodiments, combinations can achieve synergistic results, e.g., greater than additive results, e.g., at least 25, 50, 75, 100, 200, 300, 400, or 500% greater than additive results. In aspects, multiple different CEPESCs or CEPESCs and AACs are administered/applied to TR(s). E.g., CEPESCs or CEPESC(s) and AAC(s) can be administered/applied 2-12, 2-10, 2-8, 2-6, 2-5, or 2-3 times to TR(s). Typically, CEPESC(s) are applied 2, 3, 4, or 5 times to TR(s). In aspects, AAC(s) and CEPESC(s) are administered/applied simultaneously, or substantially simultaneously (e.g., within the same 30 minutes, 20 minutes, 15 minutes, or 5 minutes). In aspects, AAC(s) and CEPESCs are administered/applied at different times, e.g., within 1-8 hours, 1-2 hours, 1-3 hours, or 1-4 hours, or are separated by 1-12 days, e.g., 1-8 days, 1-7 days, 1⁻⁵ days, 1-4 days, 1-3 days, or 1-2 days, and in aspects such applications are separated by 1-12 weeks, 2-12 weeks, 3-12 weeks, 2-18 months, 2-6 months, etc.

Methods of the invention can be applied to any suitable individual target recipient (TR) (aka, “hosts” or “subjects”) or population(s) of TR(s). TR(s) can be any vertebrate that is infectable by aHVs. In aspects, TR(s) are mammals. In aspects, TR(s) are non-mammalian vertebrates, e.g., birds. In aspects, TR(s) are humans (sometimes also referred to as “patients”). In aspects, TR(s) are non-human animals (NHAs). In aspects, NHAs are livestock/farm animals (e.g., cows, cows, sheep, chickens, goats, geese, bison, ducks, turkeys, and the like), sport animals, companion animals/pets (such as cats, dogs and horses), wild animals (e.g., in methods in which efforts are made to prevent transmission of viruses or other pathogen(s) to human(s) or other animals to preserve populations), zoological animals (e.g., primates), and animals used in laboratory studies (e.g., rodents such as mice, rats, and the like, rabbits, etc.). In aspects, TR(s) are ungulates. In aspects, TR(s) are even-toed ungulates (order Artiodactyl) (e.g., cows, sheep, goats, or pigs).

In aspects, TR(s) are NHA(s) that do not express HVEM or a known HVEM analog. In aspects, such TR(s) include horses or pigs/swine (for which HVEM homologs have not yet been identified and may not be present) or cows (for which no HVEM homolog has been identified and which are expected to not possess a HVEM homolog). In aspects, OSMGAOA gDP(s) delivered to such TR(s) lack any HVEMBD or comprise a HVEMBD with significantly reduced affinity for known HVEM gDR(s). In aspects, CEPs expressed in such methods comprise 1+ NGDPCIs, such as a PD-L1 trap or an anti-PD-L1 Ab. In aspects, SMGAOA gDP(s) in CEPs delivered to such NHA(s) bind N1 or N2.

In aspects, TR(s) are NHAs known to express HVEM (e.g., dogs, mice, African Green Monkey, and guinea pigs). In aspects, OSMGAOA gDP(s) in CEPs expressed in such TR(s) comprise HVEMBD(s). In aspects, SMGAOA of such gDP(s) exhibit CI properties in such TR(s). In aspects, SMGAOA of such gDP(s) exhibit enhanced HVEM binding with respect to WT gDP(s), e.g., the WT gDP of the virus that infects the species of the TR.

In aspects, TRs are not immunologically suppressed TRs (ISTRs). In aspects, TRs are not ISTRs that undergoing a condition associated with DOS checkpoint inhibition to DCAs. For example, TRs that have well-developed cancers often have significant checkpoint inhibition. In such TRs although endogenous CAg(s) are present, ICs are either not active or not capable of inducing effective CE(s) due to the checkpoint inhibition. In such TR(s), particularly in HVEM-expressing TR(s) delivery of gDP(s) can induce IR(s)/CE(s) by, in significant part, mostly, or generally through relieving BTLA/HVEM checkpoint inhibition. Methods involving such checkpoint inhibition-blocking gDP(s) are an aspect of the invention. However, delivering CEPESCs to TR(s) that do not exhibit such a form of immunosuppression is another aspects, as the ability to induce effective IR(s)/CE(s) in TR(s) that do not contain such pre-built up blocked IR(s) is a meaningfully different physiological context in which to induce IR(s)/CE(s) and to treat or prevent disease.

In aspects, CEPESC(s) are delivered to TR(s) that have been treated with a “leaky vaccine” or TR(s) for which the standard of care only affords a leaky vaccine option (e.g., pigs with respect to PCV or PRRSV or humans or other animals with respect to influenza). aspects relating to leaky vaccines are described elsewhere. In aspects, the TR(s) are NHA(s) and the CEPESC is administered to a defined population, such as a herd associated with an area, ranch, farm, or other facility/location. In aspects, delivery of CEPESC(s) DOS reduces leaky vaccine effects, such as spread of the condition through the population, through neighboring populations, spread to new members of the population, and the like. In aspects, CEPESCs are delivered to TR(s) for which the available standard of care produces leaky vaccine effects. The inventors have discovered that the CEPESCs and methods of this disclosure can unexpectedly significantly reduce or eliminate leaky vaccine effects and provide a meaningfully different alternative in prevention or treatment of DCA-associated diseases, e.g., pathogen-associated diseases, over currently marketed “leaky vaccine” vaccine products and therapeutics. In aspects, such TR(s) are horses and the method is applied to horses previously/currently treated with an EHV-1 or EHV-4 leaky vaccine or is applied prophylactically to horses to keep the horses from developing EHV-1 or EHV-4, which currently would be “leaky vaccine” DCAs. The leaky vaccine status of EHVs is described in, e.g., Allen G P et al. “Equid herpesvirus-1 (EHV-1) and ˜4 (EHV-4) infections.” In: Coetzer, J A W and Tustin, R C (Eds.), INFECTIOUS DISEASES OF LIVESTOCK. 2nd Edn. Oxford Press: Cape Town; 2004. pp. 829-859.

Delivering EA(s) of CEPESC(s) to TR(s) DOS induces IR(s), typically causing DOS CE(s), and often resulting in DOS evidence of treatment or prevention of DCA-associated disease(s)/condition(s). IR(s) include, e.g., DOS T and/or B cell responses, i.e., cellular and/or humoral immune responses, e.g., cytotoxic T cell responses, innate immune response, and innate trained IRs (ITIRs) (e.g., DC or NKC response(s)). Humoral immunity involves plasma cells (activated B cells) and memory B cells, production of Ag-specific Abs by such B cells, & typically resulting neutralization or opsonization of DCAs. Cellular immunity involves the production of effector TC, mTCs, & NKCs, including CD4+T & CD8+ TCs, responsible for direct (cell-to-cell) or indirect (cytokine-mediated) destruction of infected cells. IR(s) include DOS “adaptive” IR(s), “innate” IR(s), and “innate trained” IR(s). Such aspects of the immune system are known. Briefly “adaptative immunity” means IR(s) carried out by hematopoietic T cells and B cells derived from the lymphoid lineage. APCs, particularly DCs, recognize DCA(s), process Ag(s), and present Ag(s) via MHC to naive T cells (Th0), Ag-specific TCs are activated leading to expansion into effector TCs (Th1, Th2, and Th17 cells), which prime or implement humoral immunity, pro-inflammatory cytokine secretion, and activation of other ICs. Long half-life T cells become mTCs. “Innate immunity” comprises hematopoietic cells including mast cells, neutrophils, and eosinophils derived from myeloid lineage, and involves non-specific IR(s), such as non-specific DCA-associated cell phagocytosis (e.g., in response to immunostimulatory signals activated neutrophils express Fc and complement receptors allowing increased phagocytosis and activated macrophages secrete proinflammatory cytokines/chemokines such as MIP-10). “Innate trained immunity” (aka “trained immunity” or “innate immune memory”) refers to the ability of cells typically of hematopoietic lineage e.g., macrophages, monocytes, DCs, & NKCs, to exhibit DOS enhanced IR(s) in reencountering DCAs, resulting in enhanced inflammatory response(s) and cytotoxic response(s). In aspects, IR(s) comprise DOS memory IR(s). In aspects, memory IR(s) resulting from EA(s) of CEPESC(s) are significantly greater than those obtained with comparable DNA vaccines, PPT vaccines, or even constructs of the Wistar Art. E.g., IR(s) arising from EA(s) of CEPESCs can be increased by about 20-500%, e.g., about 33-300%, e.g., about 50-250%, or about 75% to about 200% (e.g., about 20-200%). E.g., IR(s) can be increased by at least about 60%, 70%, 85%, 90%, 95%, 97%, 110%, 120%, 125%, or 130%. In aspects, IR(s) increased by at least about 1.5-fold, 2.0-fold, 2.5-fold, 3.0-fold, 4.0-fold, 5.0-fold, 7.0-fold, 8.0-fold, or ≥˜10.0-fold. In aspects, delivery of EA(s) of CEPESCs can result in, e.g., an at least about 2-fold (200%) increase in antigen presentation, such as an ≥˜5-fold increase in antigen presentation, ≥˜10-fold increase in antigen presentation, ≥˜20-fold increase in antigen presentation, and at least about 30-fold increase in antigen presentation, an at least about 50-fold increase in antigen presentation, an at least about 100-fold increase in antigen presentation, or more (such as an about 2-about 100, about 10-about 100, about 20-about 100, about 2-80, about 5-80, about 10⁻⁸⁰, about 2-50, or about 5-50 increase in antigen presentation) (measuring Ag presentation is exemplified in Mahnke K, et al. J Cell Biol. 2000; 151(3):673-684).

In aspects, CEPESCs induce DOS IR(S) in bystander ICs. In aspects, such IR(s) comprise DOS IR(s) in bystander DCs. In aspects, such IR(s) comprise DOS IR(s) in bystander TCs. Aspects of bystander immunity and ICs are described in, e.g., US20200325182. IRs can comprise IC phenotype skewing, expansion, maintenance, differentiation, dedifferentiation, survival, proliferation, cytotoxicity, persistence, and/or cell recall/memory, thereby improving the therapeutic potential of the immune cells. IRs can comprise DOS increases in IC proliferation, cytotoxicity, or persistence. In aspects, IR(s) comprise an increased number or relative ratio of naive T cells (Tn), stem cell memory T cells (Tscm), and/or central memory T cells (Tcm), and/or improved cell proliferation, cytotoxicity, cell recall, and/or persistence in comparison to the T cells without the same treatment. In some embodiments, the number of Tn, Tscm, and/or Tcm is increased by at least 10%, 20%, 30%, 40%, 50%, 60%, 70%, 80%, 90%, or more, or increased by at least 2, 3, 4, 5, 10, 15, or 20-fold, or more, compared to the number of Tn, Tscm, and/or Tcm in the cell population without the same treatment. In aspects, the population or subpopulation of NK cells contacted with one or more of said modulating agents comprises an increased number or relative ratio of adaptive (or memory) NK cells, and/or improved cell proliferation, cytotoxicity, cell recall, and/or persistence in comparison to the NK cells without the same treatment. In aspects, the number of adaptive NK cells is increased by at least 10%, 20%, 30%, 40%, 50%, 60%, 70%, 80%, 90%, or more, or increased by at least 2, 3, 4, 5, 10, 15, or 20-fold, or more, compared to the number of adaptive NK cells in the cell population without the same treatment. In aspects, EA(s) of CEPESC(s) results in IR(s) comprising DOS increased number or activity of activated TCs (CD134+, CD137+, and FOXP3+); increased number or activity of activated NKCs (e.g., NKp46+ NKCs), increased eosinophil counts, improved Teff to Treg ratio, increased active phenotype monocytes (CD16+ and CD68+), or increased total monocyte counts.

In aspects, IR(s) are induced in cells that are not classified as ICs, but which are capable of IR(s), such as fibroblasts. In aspects, CEPESCs induce IR(s) in cells of or arising from myeloid progenitors or lymphoid progenitors. In aspects, IR(s) are induced in non-IC endothelial cells.

In aspects, CEPESCs induce DOS IR(s) in adaptive ICs, innate ICs, or innate trained ICs. In aspects, CEPESCs induce humoral IR(s), cellular IR(s) (e.g., cytotoxic IR(s) or cytokine-expression IR(s)), or both. In aspects, CEPESCs induce IR(s) in APCs, e.g., DCs. In aspects CEPESCs induce IR(s) in T, NK and NKT cells. In aspects, CEPESCs induce IRs in BCs, TCs, and DCs. In aspects, CEPESCs induce DOS IRs in, i.a., BCs, TCs, DCs, and NKCs. In aspects, IR(s) in TCs include both CD4 TCs and CD8 TCs. In aspects, such IR(s) comprise an increase in the number of such cell(s) in TR(s) or an increase in the relative ratio of such cells (e.g., an increase in the number or ration of stem cell memory TCs or central memory TCs).

In aspects, IR(s) comprise adaptive IC IR(s). In aspects, such IR(s) comprise DOS TC IR(s), BC IRI(s), or both. In cases, BC IR(s) comprise DOS increased Ab production and other BC IR(s) such as opsonization. E.g., in aspects, CEPESCs result in an increase in TC frequency of at least 2×, at least 2.5×, at least 3×, at least 3.5×, or more over baseline, a suitable period after initial administration or after 2 or 3 administrations of CEPESCs (e.g., 2-16, 2-12, or 2-10 weeks after initial/boost administration).

CEPESCs can induce TC IRs in several type of T cells at various developmental stages, including but not limited to, CD4+/CD8+ double positive T cells, CD4+ helper T cells (e.g., Th1 and Th2 cells), CD8+ T cells (e.g., cytotoxic T cells), stem cell memory T cells (Tscm), peripheral blood mononuclear cells (PBMCs), peripheral blood leukocyte (PBL)-associated TCs, tumor infiltrating lymphocytes (TIL) TCs, memory T cells, naive T cells, regulatory T cells, gamma delta T cells (γδ T cells), as well as Th3, Th17, Th9, or Tfh cells. Additional types of memory T cells include cells such as central memory T cells (Tcm cells), effector memory T cells (Tem cells and TEMRA cells). In aspects, TCs are isolated/modified TCs, e.g., T cells modified to express a T cell receptor (TCR) or a chimeric antigen receptor (CAR). In aspects, CEPESCs DOS enhance the ratio of T effector cells (e.g., T effector cells specific to Ag(s) in CEPs) to regulatory T cells (Tregs), e.g., in an organ of a TR (e.g., the spleen), in tumor(s), or in the TR overall.

In aspects, CEPESCs DOS enhance CD8 TC IR(s). In aspects, CD8 TC IR(s) comprise DOS Th1-cytokine expression, such as DOS IFN-γ. In aspects IR(s) comprise DOS CD8 TC production of granzymes or perforins. In aspects, IR(s) comprise DOS TC killing of DCA-associated cells. In aspects, CD8 TC IR(s) comprise DOS TC expression of TNF-α, and IL-2. In aspects, IR(s) comprise detectable increase in CD8 TCs or specific CD TC(s) (e.g., CD8 TCs specific for Ag(s) in CEPs). In aspects, IR(s) comprise an increased proportion of CD8 TCs among TCs or as compared to DCA-associated cells in a TR. In aspects, the number of Ag-specific CD8 TCs, the amount of Th1 cytokines expressed in TRs, or both, in response to EA(s) of CEPESC(s) are enhanced by 1.5×, 2×, 2.5×, 2.75×, 3×, 3.25×, 3.5×, 4×, 4.5×, or 5×. In aspects, IR(s) comprise other DOS enhancements of CD8 TC IRs, which are known (SFE Cox M A, Zajac A J. Shaping successful and unsuccessful CD8 T cell responses following infection. J Biomed Biotechnol. 2010; 2010:159152).

In aspects, delivery of EA(s) of CEPESC(s) induces DOS CD4 TC IR(s). In aspects, CD4 TC IR(s) comprise Th1 or Th2 CD4 IR(s) (e.g., expression of IFN-γ and expression of IL-4, IL-5, and IL-13, respectively). In aspects, CD4 TC(s) comprise DOS upregulation of ligands, such as CD80 and CD86, on DCs. In aspects, CEPESCs comprise known non-DCA-associated (universal) CD4 epitope Ag(s), such as PADRE epitopes or natural tetanus sequences. In aspects, universal or other CD4 epitopes are fused to CD8+ T epitopes. In aspects, such CEPs also include other known or predicted Ag-specific CD4 TCEs. Such aspects are described elsewhere. In aspects, CEPs further comprise CD40 ligand PPTs, which can enhance TC IR(s) and DC IR(s).

In aspects, CEPESC(s) induce DOS CD4 and CD8 TCs. In aspects, the CD4 and CD8 TC IR(s) are balanced in a manner that DOS enhances CD8 mTCs in TRs as compared to in the absence of CD4 IRs or significantly lower CD4 IRs. In aspects, IR(s) comprise a DOS increase in the number of CD8 T cells phenotypically evidencing they were primed and maintained in the presence of CD4 TCs, e.g., by expression of markers CD62^{lo/hi}, CD122^{hi}, and CD127^{hi} (versus CD62L^{lo}, CD122^{lo}, and CD127^{lo}. Thus, an enhanced ratio of the former to the latter is an aspect, as is DOS enhanced CD8 TC cytokine expression, or both. In aspects, CD4 TC IR(s) comprise DOS expression of IL-2 and IL-21, upregulation of B lymphocyte-induced maturation protein 1. In aspects, CD4 TC IR(s) comprise DOS increases in mTC populations.

In aspects, delivering EA(s) of CEPESC(s) induce innate trained immune cell (ITIC) IR(s). In aspects, ITIC IR(s) include DOS increased population, activation, or both of NKC(s) (e.g., granzyme B⁺ NK cells), monocytes (e.g., HLA-DR⁺ monocytes), macrophages, NKCs, DCs (e.g., CD8α+ DCs), NKT cells (e.g., Type I NKT cells), or combinations. In aspects, such IR(s) comprise increases in concentration(s) of such cells in tissues/organs of a TR, increased numbers of SMGAOA of such cells, increased activation of OSMGAOA of such cells, or combinations. In aspects, ITIC IR(s) comprise DOS enhanced production of IL-6, TNF-α, IL-1β, IL-18, IL-12, L-1b GM-CSF, IL-23, or combinations. In aspects, such IR(s) comprise enhanced IFNg production by NKCs, enhanced production of IL-12 by DCs, or both. In aspects, IR(s) comprise DOS enhanced production of IL-2, IFNg, TNF-α, IL-4, or combinations. In aspects, DC(s) primarily, generally, or only promote Th1 CD4 TC IR(s). In aspects primarily, generally, or only promote Th2 CD4 TC IR(s). In aspects, CEPESCs DOS induce DCs to promote both Th1 and Th2 TC IR(s). In aspects, CEPESCs comprise IL-12 PPTs and DOS induce Th1-associated DC IR(s). In aspects, IR(s) comprise DOS polarization of ITIC(s) or other IC(s). In aspects, IR(s) comprise repolarization of macrophages. In aspects, IR(s) comprise DOS increases in population, activity, or both of NKCs that are CD3- and CD56+, expressing and have at least one of CD57+, NKG2C and CD57, and optionally, CD 16, but exhibit DOS low expression (or no detectable expression of) PLZF, 1=SYK, FceRy, FcsRy, TIGIT, lPD1, CD7, or CD161, and further may exhibit DOS high levels of LILRB1, CD45RO, or CD45RA. In aspects, IR(s) comprise DOS enhanced proliferation or activation of NKCs exhibit DOS enhanced NKG2C or CD57, or CD57, CD16, NKG2C, CD57, NKG2D, NCR ligands, NKp30, NKp40, NKp46, NKG2A, or DNAM⁻¹. In aspects, IR(s) comprise DOS NKC expression of perforin or granzyme. Other various aspects of ITIC IR(s) associated with CEPESC delivery are described elsewhere.

In aspects, CEPESCs induce DOS IR(s) in non-immune cells. In aspects, such non-IC IR(s) are in addition to DOS IR(s) in IC(s). Examples of non-IC(s) that can exhibit IR(s) include mesenchymal stem cells which can comprise Toll-Like Receptors (TLRs) and exhibit IR(s) comprising production of pro-inflammatory cytokines IL-8, MCP-1, and IL-6; hematopoietic stem cells, in which IR(s) comprise production of pro-inflammatory cytokine IL-1β, production of GM-CSF; epithelial stem cells which express TLRs and exhibit IR(s) comprising DOS production of immunomodulator factors and antimicrobial peptides; intestinal stromal cells, microglial cells, and fibroblasts (in which IR(s) comprise production of antimicrobial peptides, cytokines, chemokines, and growth factors). CEPESCs can induce DOS IR(s) in OSMGAOA of such cells. PMCs are provided in, e.g., Hamada A, et al. Front Microbiol. 2019; 9:3225.

In aspects, CEPESCs result in DOS induction/enhancement of IR(s) in TCs, BCs, and ITICs. In aspects, such IR(s) in ITIC(s) comprise IR(s) in DCs, NKCs, or both. In aspects, such TC IR(s) comprise DOS CD8 and CD4 IR(s). In aspects, such IR(s) also comprise DOS induction of adaptive memory IR(s) and innate trained memory IR(s). In cases, compositions/methods cause DOS induction of immune memory IR(s). In aspects, delivery of EA(s) of CEPESC(s) induces memory IR(s) that are DOS improved as compared to corresponding PPT Ag(s), DNA vaccines encoding such Ag(s), or even Wistar Art constructs. In aspects, such IR(s) comprise a DOS increase in the number of ICs that exhibit a memory phenotype. In aspects, such IR(s) comprise a DOS faster IR(s), more extensive IR(s), or both, when the TR is re-challenged with a DCA or similar DCA. In aspects, memory IR(s) comprise adaptive memory IR(s) (e.g., enhanced numbers of memory T cells). In aspects, memory IR(s) comprise innate trained memory IR(s) (e.g., enhanced memory NKC IRs, enhanced memory DC IRs, or other enhanced ITIC IRs, such as memory macrophage IRs). In aspects, memory IR(s) comprise both adaptive memory IR(s) and ITIC memory IR(s). In aspects, memory IR(s) include non-IC memory IR(s). In aspects, memory ITIC(s) exhibit epigenetic or transcriptional reprogramming change(s) that reflect a memory immunity state (e.g., histone modifications). Such aspects of ITIC memory adaptable to such aspects are described in Hajishengallis G, et al. Adv Exp Med Biol. 2019; 1197:11-26. In aspects, memory IR(s) comprise DOS enhancement in memory BC IR(s), such as faster or enhanced Ab production to re-challenge with a DCA. In aspects, memory IR(s) are greater than initial response(s) (e.g., anti-DCA Ab concentration is greater in a memory BC IR). In aspects, such greater memory IR(s) is/are at least 5×, at least 10×, at least 20×, at least 40×, at least 50×, at least 75×, at least 90×, at least 100×, at least 200×, at least 500×, at least 750×, or even about 1000×initial IR(s), in aspects being maintained after other signs of IR subside. In aspects, memory IRs comprise increases of types of memory ICs of at least 5%, at least 10%, or more, in aspects after initial IRs subside. In aspects, memory IR(s) comprise DOS enhanced population of memory phenotype IC(s), even after initial IR(s) have subsided. In aspects, challenge of CEPESC vaccinated TRs with antigen(s), DNA vaccine, or the like results in a DOS mTC response within about 5 days of such challenge, optionally followed by a mBC response maximizing around 1 month after such challenge. In aspects, memory BC IR(s) comprise production of Abs with higher affinity for Ag(s) than initial IR. In aspects, memory IR(s) comprise DOS enhanced levels of effector memory T cells, central memory TCs, resident TCs, or combinations. In aspects, memory IR(s) comprise memory IR(s) in gamma delta TCs. In aspects, memory IR(s) comprise enhanced CTL IR(s) as compared to initial CTL IR(s). In aspects, memory IR(s) are exhibited in non-ICs. In aspects, such memory IR(s) comprise accelerated wound healing. In aspects, EA(s) of CEPESC(s) DOS enhance NKCs with memory markers, such as DOS enhanced CXCR6 expression, FcγR (including CD16) expression, Ly49H expression, but lacking/lower expression of intracellular γ-signaling chain and KLRG1⁻. In aspects, memory IR(s) include DOS increases in number of TCs with memory phenotypes/markers (e.g., CD127^{hi}KLRG-1^{lo}CD62L^{hi}Bcl-2^{hi}). In aspects, memory IRs induced by EA(s) of CEPESC(s) comprise multiple-facets of memory IR(s), such as combinations of SMOA of memory TCs, memory BCs, memory DCs, memory NKCs. Aspects of memory IR(s) that can be generated by EA(s) of CEPESC(s) and related PMCs are in, e.g., US20200325182. In vitro IR(s) induced by EA(s) of CEPESC(s) can be measured by any suitable technique. Methods include flow cytometry, target cell lysis assays (e.g., chromium release assay), the use of tetramers, etc. (see US20200325182).

In aspects, CE(s) associated with delivery of EA(s) of CEPESC(s) comprise reducing the incidence of a DCA-associated disease (e.g., a pathogenic disease or cancer) in TR(s); ameliorating symptoms of a disease; increasing survival or chances of survival (e.g., as determined by significant results in clinical study(ies)); stabilizing/halting reducing or minimizing progression of a disease; inducing, expediting or enhancing a state of remission or recovery; enhancing the efficacy or protective effects of CCCs/AACs; decreasing the number or frequency of relapse episodes; increasing latency between symptomatic episodes; reducing the severity, extent, or duration of episodes/disease/symptoms; reducing the number of symptoms or ameliorating symptoms; or combinations.

In aspects, delivery of CEPESCs are AW DOS reduced AE(s) as compared to corresponding PPT Ag compositions, DNA vaccines, or even Wistar Art constructs. In aspects, Grade 3 AEs (severe AE(s)), Grade 4 AEs (life threatening/disabling AE(s)), or both, or serious grade 3/4 AEs-SAEs) associated with CEPESC(s) are less than 25%, less than 20%, less than 15%, less than 12%, less than 10%, ≤˜8%, or less than 5% in TR(s) (e.g., as determined by 1-4, e.g., 2-3 clinical studies). (SFE Smit et al. Journal of Clinical Oncology 2017 35:15 suppl, 2544-2544 for grading guidance).

In aspects, CEPESCs are delivered to TR(s) prior to occurrence, emergence, or detection of a DCA-associated disease (DCAAD). In cases, TR(s) lack(s) any history of autoimmune disease or cancer prior to initial CEPESC delivery. In aspects, TR(s) lack(s) any history of pathogen infection prior to treatment with a pathogen Ag-expressing CEPESC. In cases, EA(s) of CEPESC(s) are delivered therapeutically, e.g., to a subject with a preexisting condition, e.g., a subject infected with a pathogen, has a cancer, or both.

In aspects, CEPESCs DOS reduce cytokine expression-associated AEs (CEAAEs) in TR(s) (generally or compared to competitive product(s)) and methods of delivering CEPESCs can comprise steps to reduce risk of CEAAE(s), including delivering CEPESC(s) incorporating such features. Examples of such cytokine-AE-reducing features (CARFs) associated with CEPESCs include deimmunization of OSMGASAOA EP(s) of CEPs, e.g., by GSRVs, elimination of non-DCA associated BCEs; or combinations; selection of Ag(s) to primarily, generally, or at least substantially direct TC IRs, e.g., to Th1 TC IRs or CD4 Th1 TC IRs; and combinations. Methods directed to reduce CEAAE risk(s) comprise assessing TR(s) for such risk(s), monitoring TR(s) for the development of such conditions or indicators of risk factor(s) for such conditions, and changing dosing regimens, medicament(s), applying AACs, or combinations thereof in response to incurring conditions associated with CEAEE(s) or risk of CEAEE development. In aspects, such CEPESCs, methods, or combination of both ACB DOS reduced cytokine-related AEs than treatment with compositions lacking present CARF(s), methods lacking CEAEE-risk reduction step(s) or lacking any employed combinations. In aspects, such reduced risks comprise reduce occurrence of Vaccine-Associated Enhanced Respiratory Disease (“VAERD”) or conditions thereof (e.g., DOS dysregulation of proinflammatory or anti-inflammatory cytokine(s), e.g., DOS increased IL-1b levels or IL-2 levels, DOS decreased IFNa or IL-10 levels, and DOS associated AEs, e.g., DOS increased rate, duration, or severity of pneumonia, anorexia, lethargy, coughing or other respiratory disease(s), dyspnea, fever, or combinations). PMCs related to VAERD are described in Rajão D S et al. J Gen Virol. 2016; 97(7):1489-1499; Gauger P C et al. Viral Immunol. 2013; 26(5):314-321; Gauger P C et al. Virology. 2014; 471-473:93-104; and Guager, P. C. 2012. Iowa State University Graduate Thesis. In aspects, such methods are associated with the treatment or prevention of influenza, RSV, dengue viruses, coronaviruses, Ross River virus (RRV), human Metapneumovirus (hMPV), immunodeficiency viruses (such as HIV, SIV, or FIV), Equine Infectious Anemia virus (EIAV); measles virus; West Nile (WNV); Japanese Encephalitis virus (JEV), Lactate Dehydrogenase Elevating virus (LDEV), 64 Rabies virus (RV), Aleutian Disease virus (ADV), PRRSV, PCV, or ASFV or other pathogenic infections such as Mycobacterium spp., Listeria monocytogenes, Coxiella burnetii and Salmonella spp. & Leishmania spp. Other conditions similar to VAERD that can be DOS reduced by such CEPESCs and methods comprise enhanced pulmonary disease (EPD) of HMPV, enhanced respiratory disease (ERD) of RSV, atypical measles (ATM) of MV, Dengue hemorrhagic fever (DHF) or Dengue shock syndrome (DSS) of dengue virus infection, and the like. In aspects, CEPESCs exhibit DOS enhanced efficacy, DOS reduced AEs, or both in conditions CB sub-neutralizing Ab IRs, e.g., RSV, influenza, or measles virus.

In aspects, CEPESCs are delivered to TR(s) that have failed to be treated by other treatment(s) (e.g., antivirals or antibiotics) or exhibited incomplete protection from previously administered vaccines. In aspects, CEPESCs are delivered to TRs to treat a latent infection, such as a latent viral or bacterial infection, such as a MRSA or Clostridium infection. In aspects, EA(s) of CEPESC(s) DOS enhance the efficacy of AAC(s) or CCC(s), e.g., antibiotics or antivirals for therapeutic use or other vaccine(s). In aspects, pathogen(s) treated by EA(s) of CEPESC(s) comprise those pathogen(s) described elsewhere. In aspects, delivery of EA(s) of CEPESC(s) DOS reduces spread of pathogenic DCA(s) in a population or from population-to-population or species-to-species (e.g., from dogs to humans, pigs to humans, etc.). In aspects, CEPESC(s) provide “sterilizing immunity” against DCA(s) in DOS TR(s). Examples of such pathogen(s) include IV in various TR(s) (treated/prevented by, e.g., CEPESCs comprising H3/H1 Ag(s), or H3N8, H3N2, H1N1, H5N1, H3N1, or H1N2 Ag(s) such as NP, HA (e.g., HA stalk), or M1 Ag(s)), PCV, PRRSV, ASFV, & various forms of COV in humans or NHA(s)).

In aspects, CEPESCs comprise PRRSV Ag(s). In aspects, PRRSV CEPESCs induce different or enhanced anti-PRRSV IR(s) as compared to Porcilis PRRS from Merck, Ingelvac PRRSFLEX EU from Boehringer Ingelheim, Amervac-PRRS from Hypra, Pyrsvac-183 from Syva, Fostera PRRS from Zoetis, Ingelvac PRRS MLV/Ingelvac, or PRRSATP from Boehringer Ingelheim or even regarding to a Wistar Art constructs lacking feature(s) of the present constructs (e.g., enhanced nectin-1 binding, no-HVEMBD, soluble gD status, incorporation of gDSS, incorporation of PTPS(s), deimmunized Ag(s), inclusion of ITICITMs, such as ITICSTAPs, such as EAT-2 or EAT-2+ SAP, association with CaPNPs, and expression from NAM(s) comprising EEI(s), etc.). PMCs related to PRRSV are disclosed in Li B et al. Emerg Infect Dis. 2009; 15(12):2032-2035.

In aspects, delivering EA(s) of CEPESC(s) DOS enhances efficacy of IR(s), duration of IR(s), or both regarding pathogenic DCAs, e.g., ASFV.

In aspect(s), such results are obtained with 3 administrations or less or 2 administrations or less. In aspects, anti-ASFV CEPs comprise ASFV p32, p54 or p72 Ags or other ASFV Ag(s).

In aspects, delivery of such CEPESCs results in DOS enhanced DC, NKC, CTL, CD4+, and TCR-γδ T-cell responses in TR(s) as compared to use of non-CRA/PCRA Ag(s). In aspects, CEPESC(s) provide protective effects against DCA challenge or re-challenge independent of the number of exposure(s) over the lifetime of TR(s). In aspects, CEPESC(s) are administered as a treatment when a latent pathogen is detected to emerge from latency. E.g., CEPESCs comprising Leishmania parasite Ag(s) to induce CE(s) (e.g., treatment of cutaneous leishmaniasis skin sores, visceral leishmaniasis internal organ damage, or both). In aspects, CEPESCs are administered to both human and NHA in close proximity for the same DCA, e.g., in methods companion animals and humans living or working in proximity can be treated with compositions expressing Ag(s), such as anti-leishmaniasis Ag(s), anti-coronavirus Ag(s) or anti-influenza Ag(s), or humans and NHA livestock animals can receive CEPESCs comprising anti-PCV Ag(s), anti-PRSV Ag(s), and the like.

In aspects, EA(s) of CEPESC(s) are delivered to treat or prevent cancer. In aspect(s), CEPESC(s) are delivered to TR(s) that exhibit precancer conditions. In cases, compositions are administered where cancer progression or physical indicator(s), imaging indicator(s), gene expression indicator(s), immunohistochemistry indicator(s), or immunodiagnostic indicator(s) of cancer(s) is/are detected. In aspects, delivery of CEPESC(s) reduces one or more aspect(s) of cancer progression. In cases, such aspects of cancer progression that are DOS reduce(d) include (1) rate of increase of precancer or cancer cells or markers thereof; (2) number or size of cancerous growths (lesions, tumors, and the like); (3) probability, timing, or degree of the next phase of cancer progression; (4) reduction of cancer metastasis or invasiveness; or (5) combinations. In aspects, treatment of cancer by such methods DOS results in reduction in the rate of tumor growth, cancer spread, or even reduction in tumor number, average tumor size, etc. In aspects, CE(s) related to such aspects(s) comprise DOS prolonged survival, reduced risk of near-term fatality (e.g., in 1⁻⁵ years), reduction of symptoms associated with cancer (e.g., cancer-associated pain), improvement in quality of life, etc. In aspects, CE(s) comprise DOS reducing the size of an established tumor or lesion in the subject (e.g., reducing size of a tumor or average size of tumor(s) by at least 20%, at least 30%, at least 50%, e.g., about 60-100%, about 75-100%, or about 85-100%). In aspects, CEPESCs DOS enhance survival rate(s), survival time(s), or both, alone or AW CCEPM(s) or CCEPC(s) (e.g., increasing average survival time by least at least 3, 4, 6, 8, 9, 12, 18, 24, 30, 36, 42, 48, 54, or 60 months in TR(s) diagnosed with the relevant cancer). In aspects, CEPESC(s) are used to treat a cancer is a relapsed or refractory cancer. In aspect(s), CEPESC(s) are delivered to an NHA TR that is associated with susceptibility to certain cancer(s) (e.g., golden retrievers/lymphoma, gray horses/melanoma, and other examples described elsewhere). In aspects, CEPESCs are delivered to treat bladder cancer/TCC. In aspects, such methods are performed on humans or dogs. In aspects, CEPESC(s) comprising CAg(s) are delivered to treat lymphoma(s), e.g., in humans or in dogs. In aspects, such lymphomas are diagnosed as drug-resistant lymphoma(s) or the TR is a subject diagnosed as being of risk for developing drug-resistant lymphoma(s). Relevant PMCs, including target cancers, CCCs, etc., are provided in US20070014788 and US20200325182.

In aspects, CEPESC(s) are used to treat a cancer associated with immunologically “hot” tumors (AW relatively high amount(s) of effector IC infiltration, e.g., in a melanoma, lung cancer, head and neck cancer, etc.). In aspects, CEPESC(s) are delivered to TR(s) with “cold” tumors. In aspects, IR(s) AW delivery of CEPESC(s) include DOS initiation of TC anti-tumor IR(s), migration of IC(s) to tumor(s) (e.g., TCs, DCs, NKCs, or combinations), increased numbers of CD8 TCs (or higher ratio of CTLs/naive CD8 TCs); and the like. In aspects, IR(s) AW CEPESC(s) comprise DOS maturation of DCs (e.g., in response to CAgES CEPESC(s) or other CEPESC(s) described elsewhere). In aspects, such methods are AAW other anti-cancer therapeutic(s), e.g., chemotherapy CCEPC(s) (e.g., doxorubicin) or radiation CCEPM(s). In aspects, anti-cancer methods comprise AAW anti-cancer antibodies, such as checkpoint inhibitor Abs (e.g., anti-CTLA4, anti-PD-L1, anti-PD-1, anti-IDO, anti-IDO1, anti-CD200, anti-CD137, anti-KIR2D); cytokine(s) (e.g., IL-2 or IFNa), and the like (SFE Christofi T et al. Cancers (Base1). 2019; 11(10):1472. Other possible combination methods and CCCs, e.g., CAR-T cells, cancer-associated gene silencing agent(s), anti-cancer Abs, chemotherapeutic(s) (e.g., anti-angiogenic agents), radiotherapy, anti-cancer surgical procedures, and the like, are described in, e.g., US20200325182 and WO2017053823.

### ILLUSTRATIVE APPLICATIONS (EXAMPLES)

To even further exemplify and illuminate aspects of the invention, the following description of illustrative applications of particular aspects of the invention are provided. These Examples are meant to exemplify particular facets of the invention but should not be used to limit its scope whatsoever.

### I. GENERAL METHODS

Methods described herein are known to those of skill in the art, but exemplary applications of such methods or brief descriptions thereof are provided in the following passages or references cited therein.

Examples provided herein comprise the isolation and use of peripheral blood mononuclear cells (PBMCs/PBMC), isolation of immune cells from PMBCs, and flow cytometry or other types of analysis of such cells. PBMC isolation methods, methods for obtaining immune system cells from PBMC, flow cytometry, and other analytical methods are described in US20200325182).

Methods of T cell epitope (TCE) identification employed in this analysis can include techniques described in, e.g., Stevenson, P. G., and Doherty, P. C. 1998. Cell-mediated immune response to influenza virus. In Nicholson, K. G., Webster, R. G., and Hay, A. J., eds., Textbook of Influenza, pp. 278-287. Blackwell Science, Oxford and Klausman, PCV-induced T cell clonal deletion in thymus, Emerging Microbes and Infections (2015) 4, e15.

### II. EXAMPLE 1—PCV CONSTRUCTS

In this Example, the test subjects are pigs and antigenic sequences encoding antigenic sequences known or predicted to induce immune responses against porcine circovirus type 2 (aka, PCV2/PCV-2) are used. PCV2 is a small DNA virus with a circular genome of approximately 1700 bp, encoding four Open Reading Frames (ORF1, ORF2, ORF3, and ORF4). T cell epitope analysis of these sequences is performed using methods described in this Section of the disclosure, which may be optionally supplemented by other methods, examples of which are provided in the Detailed Description Section of the disclosure.

Porcine dendritic cells, T cells, and B cells are isolated from porcine PBMCs. Human embryonic kidney cells (HEK293 cells), Vero cells, and canine thymus cells are used as controls. Synthetic peptide panels from PCV2 ORF expression products (including whole ORF expression products) are generated to query PBMC from PCV2 infected pigs to identify potential CRAs. These synthetic peptides are used in ELI methods employing sets of synthetic peptides representing parts of the PCV2 genome.

To perform ELI, anels of synthetic overlapping peptides representing ORFs 1,2,3 and 4 are used to query PBMC from naturally infected pigs to identify T cell targets. PCV2 and PCV3 have very small genomes so ELI is feasible for CRA screening. In some cases, CRAs are modified to generate putative editopes; e.g., putative editopes can be generated by preparing nucleotide sequences encoding antigenic variants edited for de-glycosylation, a removal method known to enhance immunogenicity to PCV and other viruses.

Any clinically relevant antigens identified through the ELI step are then sequenced, nucleic acids encoding the CRA(s) are prepared or isolated from PCV2, and CRA-containing construct(s) are generated in a similar manner to the two specific constructs described below in this Example. It is anticipated that one or more polyepitope-encoding DNA sequences will be generated from the ELI screen and expression of PCV2 ORFs or portions thereof.

The antigen-encoding sequences identified in the ELI screen are cloned into suitable plasmid vectors, in association with one or more gD-domain-encoding sequences to express a gD-antigen fusion protein and/or independently. In gD-antigen fusion protein-encoding constructs, the antigen-encoding sequences are positioned between nucleotide sequences encoding (1) a first gD sequence comprising residues 23-244/267 of HSV-1 gD (gD1 seq 1) and (2) a second gD amino acid sequence that comprises residues 245/268-340/392 of HSV-1 gD-1 (gD1 seq 2). A sequence encoding SEQ ID NO: 1 (a polyubiquitin chain) is optionally positioned upstream of gD-1 sequence 1 in the fusion protein coding nucleotide sequence. Also, the fusion protein can include a T2A cleavage site, such as a single T2A site downstream of gD1 seq 2. In a possible variation or extension of this plan, one or more sequences encoding variants of an above-described gD1 seq 1 sequence in which residues associated with HVEM binding and that do not impact binding to Nectin-1 and homologous receptors are removed and also used in separate constructs or replace gD1 seq 1 in the experimental plan.

In addition to sequences encoding CRAs identified in the above-referenced step, a sequence encoding the glycosylation site removal editope, ORF2A143-145, also is prepared, for insertion into plasmid vectors.

Such plasmid vectors will likely include an expression-enhancing intron, such as a CMV Intron A sequence, and a strong constitutive promoter, such as a CMV IE promoter. The plasmids will further likely comprise a reporter gene (e.g., a GFP sequence) and a non-antibiotic resistance system, such as a triclosan selection system described elsewhere herein. Cloning of plasmid containing cells can then be performed on triclosan media. The method can also comprise confirming reporter gene expression in culture to ensure proper levels of expression are occurring in vitro, in vivo, or both.

Isolated DNA plasmids are mixed with calcium phosphate nanoparticles using methods described in the Detailed Description of the Invention to form DNA-CaPNP complexes prior to vaccination of test subjects. Such vaccination is administered intranasally. Constructs to be tested in healthy animals (which will be subsequently assessed for PCV2 protection or challenged with PCV2) or PCV2-infected animals can include the following coding sequences: (a) gD1 seq 1-PCV2 CRA-gD1 seq 2 (e.g., SEQ ID NO: 1-gD seq 1-PCV2 CRA-gD seq 2-T2A SCS); (b) gD1 seq 1-ORF2A143-145-gD1 seq 2; (c) ORF2A143-145; and (d) PCV2 CRA(s).

In some cases, nucleotide sequences encoding one or more known or putative B cell epitopes from PCV2 ORF2 (which encodes the PCV2 capsid protein) are also (1) incorporated directly or indirectly (via cleavage site or linker) to the fusion protein-coding sequence, (2) separately expressed from the fusion protein-encoding nucleotide sequence-containing DNA plasmid (e.g., as a bicistronic plasmid), or encoded in a separate DNA plasmid that is administered with or in association with the fusion-protein-encoding sequence containing DNA plasmid. Examples of relevant putative epitopes are described in, e.g., Guo L, Lu Y, Huang L, Wei Y, Liu C. Identification of a new antigen epitope in the nuclear localization signal region of porcine circovirus type 2 capsid protein. Intervirology. 2011; 54(3):156-163 and Shuai J, Wei W, Li X, et al. Genetic characterization of porcine circovirus type 2 (PCV2) from pigs in high-seroprevalence areas in southeastern China. Virus Genes. 2007; 35(3):619-627, as well as in Chinese Patent Applications CN110423269A and CN110407919A. Alternatively, endogenous humoral responses to selected putative vaccine antigens in chronically infected pigs can be used to identify antigens that result in a sufficient humoral response by collecting serum and evaluating by virus neutralization against selected PCV strains e.g., PCV2a/b or PCV2d. Alternatively, ELISA can be performed using these sera and purchased or synthetic antigens (e.g., ORF2 at a minimum).

In addition, nucleotide sequences encoding a PD-L1 antagonist (e.g., a PD-L1 trap protein as described in the Detailed Description or a PD-L1 sequence obtained based on the disclosure of Richmond O, Cecere T E, Erdogan E, et al. PD-L1 expression is increased in monocyte derived dendritic cells in response to porcine circovirus type 2 and porcine reproductive and respiratory syndrome virus infections. Vet Immunol Immunopathol. 2015; 168(1-2):24-29) and an EAT-2 polypeptide (e.g., human, murine or porcine EAT-2, also as described in the Detailed Description) are obtained and either cloned into the same DNA plasmid vector as the above-referenced antigen-encoding sequences or into a separate expressible CaPNP-complexed DNA plasmid vector that it is co-administered with the above-described antigen sequence-encoding DNA plasmids (in at least some cases), such that the test subjects receive the following sequences in administered DNA plasmid(s): (a) gD1 seq 1: PCV2 CRA gD1 seq 2; (b) gD1 seq 1: PCV2 CRA: gD1 seq 2+ EAT-2; (c) gD1 seq 1: PCV2 CRA: gD1 seq 2+PD-L1 antagonist (PD-L1a); (d) gDi sequence 1: PCV2 CRA: gDi sequence 2+PD-L1a+EA; (e) EAT-2; (f) PCV2 CRA; (g) PCV2 CRA+ EAT-2; (h) PCV2 CRA+PD-L1a; (i) PCV2 CRA+PD-L1a+ EAT-2; (j) gDi seq 1: ORF2A143-145: gDi seq 2; (k) gDi seq 1: ORF2A143-145: gDi seq 2+ EAT-2; (1) gDi seq 1: ORF2A143-145: gDi seq 2+PD-L1a; (m) gDi seq 1: ORF2A143-145: gDi seq 2+PD-L1a+ EAT-2; (n) ORF2A143-145; (o) ORF2A143-145+ EAT-2; (p) ORF2A143-145+PD-L1a+ BCEs; and (q) ORF2A143-145+PD-L1a+ EAT-2+ BCEs

Alternative versions of constructs that can be generated and tested from identified T cell antigen-encoding sequences are: (i) gD-singleORF (fragment or native); (ii) gD-multiORF (fragments of more than one PCV—ORF; (iii) gD-Editope (e.g., native antigen or fragment with glycosylation site(s) deleted); (iv) singleORF (fragment or native); (v) multiORF (fragments of more than one ORF) or (vi) GSRAgV/editope (e.g., native Ag or fragment with glycosylation site(s) deleted).

Methods can further include generation of mixtures of two or more of these types of construct-containing plasmids either in the first instance or in further iterations of the experimental method.

Endotoxin-free gigapreps are prepared for clinical studies (e.g., using GenElute™ HP Endotoxin-Free Plasmid Megaprep Kit—Millipore Sigma, St. Louis, MO, USA) comprising 7-10 mg of each candidate (an amount sufficient for pilot safety/challenge study) & formulated appropriately.

Each of the above-described treatments are provided to a group of at least 5 pigs per group. The plasmid compositions are delivered by mucosal administration of 1-2 mL of a formulation for vaccination comprising 1-200 micrograms of plasmid. Single vaccination will be employed in this study (but repeat administration can be used, e.g., one or two doses administered every 2-8 weeks). A vaccination trial is expected to include a safety phase to assess safety of the constructs for a suitable period, such as 7 days. Viral challenge is expected to take place at three to six weeks after administration. Viral challenge may be repeated with different stains of PCV-2, in order to identify constructs that are protective against a number of PCV types. Candidate strains for the challenge study can include some or all of the following—
1. PCV2d isolate JX535296, GenBank accession numbers JX679498 and JQ653449; Opriessnig 2014 J Gen Virol (2014; 95(Pt 11):2495-2503);
2. PCV2 strain GDYX, GenBank accession number JX519293; Chen et al 2012 J Virol (86(22):12457-12458); and
3. PCV2 Isolate D3276/5/16HU; (GenBank accession number: MG833033; Palya et al 2018 Virol J (15(1):185).

In a possible variation of the above-described method, PCV3 antigenic sequences are also included in the test constructs (e.g., where a broader PCV/Circovirus vaccine is sought or to employ similar principles for the development of a PCV3 vaccine comprising such a construct but with one or more PCV3 CRAs). Candidate strains that also or additionally can be used for such a PCV3 or combined PCV vaccine study include:
1. PCV3/CN/Hubei-618/2016 deposited at GenBank under the accession number KY354039; Fan et. al. Genome Announc. 2017 (5(15): e00100-17);
2. GenBank Accession Number KX458235; Palinski et. al. J. Virol. 2016 (91(1); PUBMED 27795441);
3. GenBank Accession Number KY996344; Kwon et. al. Vet. Microbiol. 2017 (207, 178-180 (2017) PUBMED 28757021); and
4. GenBank Accession No. MG014362; Fux, et. al. Virol J. 2018, 15: 25.).

PBMC are harvested at−7, 0, 7, 14, 21, 42 and optionally 56 days. PBMC will be analyzed by flow cytometry for CD4+, CD8+, NK cell, dendritic cell, gamma-delta T cell and regulatory T cell responses to vaccine antigens, or alternatively analyzed by analytical methods capable of producing similar or equivalent data, such as ELISpot analysis. Serum is collected at days 14, 21, and 42 and is used to measure antibody responses.

A third, challenge phase can further be conducted, with timing, strain selection and sample collection protocols to be determined. The performance of the vaccine candidates will be compared to at least one commercial PCV vaccine.

It is expected that several of the plasmids/constructs described above will result in a measurable and often significant B cell and T cell response to PCV, and that at least some of the constructs/plasmids will result in significant B cell and T cell responses to more than one type of PCV.

### III. Example 2—Prrsv Constructs

In this example, nucleotide sequences encoding gD1 seq 1 and/or gD1 seq 2 and an intervening PRRSV antigenic sequence are prepared and inserted into a DNA plasmid, which may comprise sequences encoding a ubiquitin (e.g., SEQ ID NO: 1) (typically positioned upstream of the antigen (for example, if gD is located at the C terminus) or gD seq 1), a T2A cleavage site (typically positioned downstream of gD1 seq 2), or both, and optionally including a CMV Intron A sequence and a strong constitutive promoter, such as CMV IE. The DNA plasmids are associated with CaPNP particles and formulations are administered to pigs using treatment and analytical approaches described in Example 1 (e.g., flow cytometric or ELISpot analysis of PBMC drawn and ELISA analysis of sera collected on the days indicated in Example 1). The three antigenic sequences tested both on their own and in the context of the gD1 seq 1-gD1 seq 2 fusion protein include (1) a native PRRSV ORF3 (SEQ ID NO:11). A nucleotide sequence encoding SEQ ID NO: 11 will be modified to generate a variant antigenic sequence in which one or more of the native putative glycosylation sites will be removed through N to D residue substitutions as reflected in SEQ ID NO: 12. A NS encoding a truncated version of SEQ ID NO:12 (limited to AAs 1-100 thereof) (SEQ ID NO: 13) will also be generated and inserted into either a gD1 seq 1: gD1 seq 2 construct or a non-gD construct. See also US20200325182. As described in Example 1, these constructs can further be combined with sequences encoding either PD-L1, PD-1 extracellular binding domain, PD-L1 extracellular binding domain, or EAT-2.

It is expected that one or more of the constructs will result in a significant level of B cell and T cell response against PRRSV.

### IV. Example 3—Asfv Cra Screening

This Example provides an overview of a screening method to identify clinically relevant antigens (CRAs) in African Swine Fever Virus (ASFV), to incorporate nucleotide sequences encoding such CRAs into a gD-antigen fusion protein-encoding construct, such as a gD1 seq 1/gD1 seq 2 construct as described in Example 1, which is in turn incorporated into a suitable plasmid DNA vector delivery system and administered to pigs in a trial to assess the suitability of the CRAs in the context of a gDAgFP expression system.

Antigens that elicit CTL responses and are expressed during the early stage of ASFV infection will be identified, fused to a gD sequence and incorporated into a DNA vaccine. Because of ASF handling restrictions, the goal is achieved in several steps, beginning with identification of viral antigens that are present at very early stages of infection and that elicit a CTL response to vaccination of healthy pigs. Fusion of these viral antigens to gD sequences and expression optionally with a checkpoint inhibitor (e.g., a PD-L1 antagonist), especially in the context of a co-expressed innate adaptive immunity immunomodulator, such as EAT-2, will boost the adaptive response by enhancing viral antigen T cell recognition, T cell proliferation and T cell memory to elicit a rapid and effective T cell response at the earliest stages of ASFV primary infection upon viral entry into host cells.

Viral particles carry antigens that are introduced into infected cells early, even though expressed at the late, post-replication stage of viral infection. Therefore, late, immediate early, early and intermediate ORFs will be included in the screen. To stimulate an innate NK cell response to vaccination, a separate plasmid will be constructed that encodes an EAT-2 polypeptide to in turn enhance the adaptive processes of APC maturation, proliferation and T cell effector and memory responses to vaccine antigens. Pools of antigens will be formulated in a CaPNP delivery system, as described above. All plasmids will be amplified via an antibiotic resistance gene-free triclosan selection system.

Safety and immunogenicity of the designed CaPNP-formulated plasmid pool vaccine will be assessed in an animal trial: Pigs will receive a combined intranasal/intramuscular prime/boost vaccination at 4 and 6 weeks of age. On a weekly basis, blood will be collected to isolate and store serum and PBMC. Serum will be used in future neutralizing antibody studies. PBMC will be used to analyze the plasmid pool-induced systemic cellular immune response: Upon in vitro re-stimulation with the vaccine antigen pool, the proliferation, cytokine and perforin production, memory cell generation and homing pattern of CD4+ T cells, CTLs, TCR-γδ T cells, and NK cells by multi-color flow cytometry will be studied. At the day of the highest immune response, the response to the four individual antigen pools will be tested. Antigens presented as gD fusions are expected to generate robust CTL responses to all or almost all of the antigens in vaccinated pigs. Including NK cell biomarkers in the analysis is intended to measure effects of innate immune stimulation by the EAT-2-expressing second, co-administered DNA plasmid. This will be the first comprehensive flow cytometry evaluation of the T- and NK cell response to such a combination of a gD-antigen fusion protein and innate adaptive immunity immunomodulator.

Genomes of more than 39 strains of ASFV have been sequenced and are available at GenBank. Based on published work and database analyses, all early and immediate early genes independent of their biological function and intermediate and late genes critical for replication and spread will be identified. It is expected that at least 50-60 highly conserved genes will be selected based on what is known about ASFV biology. Amino acid sequences for each gene selected for vaccination will be aligned and compared among all strains for which sequences are available. This will allow identifying amino acid substitutions, if any, that are naturally occurring and whether regions or domains where variation is observed have any potential T cell epitopes. If significant variation is observed for a given gene, a sequence that is most likely to yield a T-cell response will be selected. Established gene synthesis and molecular biology techniques are readily available in laboratory manuals, on the internet, and from vendors (PCR, gene synthesis, restriction digestion, agarose electrophoresis, ligation, E. coli transformation, selection of resistant colonies, miniprep DNA preparations, sequencing with primers, and megaprep DNA preparation) and commercially available reagents (enzymes, reagents, kits for cloning and DNA preparations from NEB, ThermoFisher, IDT, and Qiagen). Full-length nucleotide sequences of selected genes (Genescript) and clone into plasmid vector pMBF116 or a similar vector containing sequences for a CMV IE promoter—CMV Intron A—gD-expressing sequences for gD1 seq 1 (or a variant thereof with reduced HVEM binding as described above) and/or gD1 seq 2—and suitable reporter elements/tags (e.g., an epitope tag, a GFP reporter gene, or both) will be synthesized. The plasmid also carries the FabI gene for triclosan non-antibiotic selection and plasmid amplification. The ASFV genes will have 11 amino acids of HSV epitope tag (QPELAPEDPED (SEQ ID NO: 119)) at the C-terminus. Optional additional features such as a 2A cleavage site, a ubiquitin PTPS-encoding sequence, or both can also be added to the construct in this or later iterations of the experiment.

All plasmids made will be confirmed by DNA sequencing (Functional Biosciences, Madison, WI, USA). Endotoxin-free DNA preparations will then be made (Qiagen EndoFree Plasmid Kit). Optionally, In-Cell Western Assay is also used to confirm expression in which cells in 96-well plates are transfected, fixed to the plate and immunostained with a fluorescently tagged antibody (epitope tag) and scanned with an automated fluorescence microscope.

Equal amounts of at least 50-60 plasmids will be pooled (25 μg/plasmid) and formulated with CaPNP (Southwest Research Institute, San Antonio TX) in PBS. DNA loading will be precisely quantified. For some experimental groups, the pool will be further mixed with 25 μg an EAT-2-expressing plasmid before formulation.

Vaccine safety and immunogenicity of the designed vaccine will be evaluated in a 42 day, optionally 56-day, animal trial. After a three-day acclimatization phase, pigs will receive a combination of an intranasal/intramuscular (IN/IM) prime (day 0) and boost vaccination (day 14) with the selected pools of plasmid constructs (e.g., ASFV putative antigen only, gD seq 1+ ASFV putative CRA, or EAT-2+ gD: ASFV putative CRA fusion protein). At study termination, pigs will be sacrificed, and tracheobronchial lymph nodes collected for assessing the lung-regional immune response. Safety will be assessed by clinical monitoring; immunogenicity will be evaluated by weekly blood collections for isolation and storage of serum and PBMC. Sera will be used for neutralizing antibody analyses. PBMC will be used for in vitro restimulation assays to determine the vaccine-induced NK- and T-cell IR(s).

The following groups can be included: Group A will receive a MOCK vaccination (unformulated CaPNP only) (4 pigs); group B will receive EAT-2-expressing plasmid only (4 pigs); group C will receive gDAgFPES plasmids including AgES(s) from the synthetic gene library (8 pigs, 25 μg per plasmid); and group D will receive both, the synthetic library gDFP-expressing plasmid & EAT-2-expressing plasmid (8 pigs, 25 μg per plasmid).

To determine the T-cell response to the plasmid pool-encoded antigens, PBMC and lymphocytes isolated from tracheobronchial lymph nodes will be in vitro restimulated with a peptide pool representing the antigens used for vaccination. Activation/proliferation, cytotoxicity, cytokine production, differentiation, and homing pattern of T-cell subsets will be analyzed via multi-color flow cytometry. Activation/proliferation will be assessed by staining the cells with a proliferation dye (CellTrace™ Violet Cell Proliferation Kit, ThermoFisher, Waltham, MA) prior to a four-day cultivation period. Cytokine production will be determined using cytokine-specific antibodies (IFN-γ, TNF-α, and/or IL-2) after a 24-hour stimulation including a Golgi-inhibitor for the last four hours of culture to ensure cytokine accumulation within the cells. NK-cell and CTL cytotoxicity will be assessed by a CD107 degranulation assay following the procedures described by Mair et al. 2013. Proliferation, cytotoxicity, and cytokine stainings will be combined with antibody stainings against markers to distinguish different T-cell subsets (CD3, CD4, CD8α, TCR-γ8) as well as their differentiation status (CD45) and homing pattern (CCR7).

Only healthy piglets will be enrolled. Piglets will be randomly assigned to groups using the GraphPad online randomizer tool. Studies are planned with appropriate controls; and results will be analyzed via valid and appropriate statistical methodologies. The animal trial will be performed under BSL-2 conditions with approved Biological Use Authorizations and IACUC approval. The parameters to be measured to reflect effect of the treatments encompass different aspects of safety and immunogenicity. All in vitro studies will be performed in a BSL-2 certified laboratory. Scientific and experimental rigor are maintained in the laboratory. In vitro protocols are well established.

Data will be analyzed for normal distribution by Kolmogorov-Smirnov analysis. Non-normally distributed data will be analyzed by Mann-Whitney test. Normally distributed data will be analyzed using a repeated-measures 2-way ANOVA with time and vaccination as the 2 factors. Necropsy data will be analyzed by 2-way ANOVA with in vitro re-stimulation and vaccination as the 2 factors. Post hoc multiple comparisons will use the Dunnett's test. Differences will be defined significant (*) for P<0.05.

It is anticipated that the designed vaccine, especially in the context of EAT-2 co-expression, will induce a profound NK and T-cell response, especially in CTLs. This response will be a combination of proliferation, cytotoxicity, cytokine production and the development of tissue-homing memory cells (T effector memory cells, TEM).

PBMC isolated during the proposed study can further be used for a phase 2 leg of this project. Phase 2 will determine: i) the individual antigens or epitopes (e.g., a particular protein or epitope) within larger antigen units (e.g., an ORF or a large fragment of an ORF) that generated the detected immune responses; ii) confirmation of the immunogenicity of these antigens by in vitro re-stimulation with ASFV-infected cells; iii) the effects of gD and EAT-2 targeting/enhancement components of the system; iv) generated serum neutralizing antibody levels; and v) a challenge study using a combination of individual antigens that elicited CTL and/or NKC IRs.

### V. Example 4—Asfv Plasmid Expression Vector

This Example demonstrates configurations of DNA plasmid vectors encoding ASFV antigens that can be used in trials such as those described in Example 3, and, if proven efficacious, for vaccination or treatment applications.

Plasmids are constructed to contain unique BamHI and NheI restriction enzyme sites before a human polyubiquitin sequence (e.g., SEQ ID NO: 1) and a NotI site after the ubiquitin-encoding sequence. An ASFV CP204L-encoding sequence (encoding an ASFV p30 protein) is inserted after the NotI site followed by 2A peptide cleavage sequence. A GFP reporter gene-encoding sequence is also incorporated, followed by unique restriction enzyme sites XhoI, PmeI, EcoRI, and ApaI. The incorporation of multiple internal restriction sites and 3′ sites provide the plasmid vector with the ability to incorporate genes in a variety of locations. For example, by use of select sites, cloning without fusion can be performed (clone at BamHINheI to XhoIPmeIEcoRIApaI).; cloning to result in an N-terminal Ubiquitin fusion and no GFP (clone at NotI to XhoIPmeIEcoRIApaI) can be performed; cloning to obtain a C-terminal cleavable GFP fusion (clone at BamHINheI to AgeI) can be performed; cloning to achieve an N-terminal Ubiquitin and C-terminal GFP fusion (clone at NotI to AgeI) can be performed; and/or cloning to replace GFP with any other marker (clone at AgeI to XhoIPmeIEcoRIApaI) can be performed.

The plasmid can be tested in experiments such as those described in Example 3 or other experiments in cell lines and pigs by detecting CP204L and GFP protein expression, B-cell response, T-cell response by western blotting, ELISpot, or flow cytometry, with suitable negative controls. In one variation, the CVM IE promoter is replaced with or also contains a CAG promoter. Plasmid expression in swine can be subjected to testing prior to larger scale trials by performing small-scale (e.g., 1-3 pig trials), each pig inoculated with endotoxin-free prepped plasmids as described above under different test conditions (e.g., with CMV or CAG promoters). Examples of a sequence overview of key elements of such plasmid constructs include (1) pMBF116-CMVp-Ub-CP204L-T2A-GFP-TcnR sequence (6153 bp (SEQ ID NO: 2)) (which includes a subsequence encoding ASFV CP204L antigen, GenBank Access No. YP_009704045.1 (encoding SEQ ID NO: 23), starting at nt 1905, and (2) pMBF117-CMVp-Ub-CP204L-T2A-GFP-TcnR sequence: 6223 bp (SEQ ID NO: 3) (including a subsequence encoding ASFV CP204L antigen, GenBank Accession No. YP_009704045.1 (encoding SEQ ID NO: 23), starting at nt 1905 and a GFP sequence) (see US20200325182 for more details on the structure of these plasmids). It is expected that such plasmid vectors will result in a robust immune response in pigs and will be useful as or in generating constructs that are protective against ASFV when utilized in association with the principles described in the present disclosure.

### VI. Example 5—Canine Bladder Cancer

This Example demonstrates an approach to generating constructs for testing and demonstrating how constructs and compositions generated according to the principles and techniques provided in this disclosure can be used to demonstrate efficacy against canine bladder cancer in a clinical trial. The study comprises vaccine design, testing the vaccine for safety and immunogenicity in healthy dogs, and a clinical safety and efficacy study in dogs with naturally occurring bladder cancer. Diagnostic tests will be used to classify T cell tumor type in each patient to correlate efficacy of the vaccine combination therapy with T cell inflamed and the more difficult-to-treat T cell non inflamed tumors. A successful outcome of this study will be an immunotherapeutic checkpoint inhibitor vaccine whose efficacy can be correlated with tumor classification and response to treatment. Phase 2 will be the design and conduct of a pivotal safety and efficacy study in dogs.

Sequences encoding one or more, typically five (5) or more (e.g., eight or more, ten or more, or even twelve or more, such as 13 antigens as exemplified below) anti-tumor antigens known to be T cell targets in bladder cancer are obtained and fused with sequences encoding gD1 seq 1 and gD1 seq 2 to generate a gD: antigen fusion protein-encoding construct. The different antigen sequences can optionally be separated by linker sequences (e.g., any of the mid-sized linker sequences described in the Detailed Description or more simple linkers such as Ala-Ala linkers), cleavage sites, or both. Both DNA plasmid vectors and adenoviral vectors comprising the constructs can be generated for application such as gD-antigen fusion protein-encoding construct, such as a gD1 seq 1/gD1 seq 2 construct as described in Example 1, which is in turn incorporated into a suitable plasmid DNA vector delivery system and administered to dogs in a trial to assess the suitability of the CRAs in the context of a gD-antigen fusion protein expression system.

Alternatively, gD-antigen fusions can be cloned into adenovirus (“Ad”) C₆₈ vector derived from a chimpanzee and deleted for the E1 gene that is essential for virus replication, has no known virulence features associated with this adenovirus, and is not normally found in dogs, such that dogs are expected to not exhibit pre-existing immunity to such vectors. It will be understood that Ad vectors having similar properties (little pre-existing immunity, replication deficiency, etc.) could be used as an alternative in this type of application. As adenoviral vectors can sometimes exhibit adjuvant effects and efficient expression of constructs they can be used as an alternative to or plasmid DNA vectors in combination with plasmid DNA vectors. Plasmids generated in the study will also include “shuttle plasmids” used to incorporate constructs within Ad vectors. Antigen expression from the vectors will be assessed in human kidney HEK293 and dog thymus Cf2Th cells. Once expression is confirmed, recombinant Ad vectors can be purified using known techniques. As a further step, initial T cell immunogenicity of the AdC68 gDmulti-antigen-expressing vector can be confirmed in mouse studies as described in the Wistar Art. In some cases, EAT-2 sequence is added to the construct to enhance immunological response or a separate vector comprising an EAT-2-expressing sequence is co-administered. In some cases, a separate vector comprising a sequence encoding only FAP is also administered. In some cases, a separate vector comprising a sequence encoding a CRACC protein, such as a CRACC-Fc protein (CRACC protein fused to the Fc domain of IgG) is also administered. CRACC is an IAII that upregulates innate immunity-NK cell and DC that in turn strengthens the adaptive immune response to vaccine antigens (T and B cell) and that also exhibits checkpoint inhibition effects. Sequences for a PTPS, such as SEQ ID NO: 1 and EEI(s), such as a CMV Intron A sequence, also can be incorporated if desired, either in initial constructs or in subsequently tested constructs.

Five dogs for each treatment group will be provided. Plasmids will be administered subcutaneously at a dosage of 10⁻¹⁰⁰ μg. In cases, plasmids expressing a FAP antigen are separately co-administered with the gD-antigen-expressing construct, typically at the same dosage and by the same route. In other cases, a plasmid comprising a sequence encoding a CRACC IAII sequence also will be administered to a similar group of dogs. Thus, e.g., groups can be administered the gD-polyTAA-expressing plasmid vector alone; the gD-polyTAA vector +a FAP only-expressing plasmid, or a combination of all of the gD-polyTAA vector, the FAP only-expressing vector, and a CRACC-expressing vector. Other exemplary groups will include a combination of gD-polyTAA Ad68+ CRACC-Fc Ad68 (exhibiting a dual checkpoint inhibitor therapy—also referred to as a “doublet therapy”); gD: polyTAA Ad68+ CRACC-Fc Ad68+ FAP Ad68; or gD: polyTAA Ad68+ FAP Ad68+ EAT-2 Ad68. In cases, priming, boosting, or both with CaPNP-associated plasmids comprising corresponding constructs is performed.

PBMCs will be collected at appropriate timepoints post vaccination. Intracellular cytokine staining of PBMCs by ELISpot assays and flow cytometry analysis for production of interferon-gamma in response to the tumor-specific antigens present in the vaccine constructs will be performed. When possible, biopsy samples will be categorized by tumor type by genome expression profiling with the objective of correlating outcome to T cell inflamed or T cell noninflamed TME. T-cell-inflamed and non-T-cell-inflamed bladder tumors can be distinguished by immune gene expression profiling. See, e.g., Sweis R F, Spranger S, Bao R, et al. Molecular Drivers of the Non-T-cell-Inflamed Tumor Microenvironment in Urothelial Bladder Cancer. Cancer Immunol Res. 2016; 4(7):563-568, for relevant principles/methods and further methods provided in the Detailed Description.

From the results of these experiments, one or more constructs will be selected and advanced to scale up for pivotal studies in dogs.

### VII. Example 6—Human Bladder Cancer

Coordinated transcriptional changes in canine and human bladder cancer, including gene functions, pathways, and cytogenetic regions were highly similar at functional and pathway levels (see, Ramsay 2017). A comparison study of canine vs human TCC gene expression patterns revealed ≥43,000 genes expressed by both species, 436 were unique to TCC and common to both species (Dhawan et al., PLoS One. 2015; 10(9)).

Upon successful completion of a pivotal study of one or more of the constructs described in Example 5, human clinical safety and then efficacy studies will be performed using the Ad68 or plasmid DNA constructs comprising the vaccine successfully demonstrated in the canine model. It is expected that similar efficacy will be demonstrated in humans given relevance of the canine model of bladder cancer to the human condition.

### VIII. Example 7—Canine Influenza CRA Screening

This Example exemplifies use of the compositions and methods of the invention provided in this disclosure to provide an effective, innovative cross-protective vaccine for canine influenza (CIV). The objective of this effort is to develop a vaccine that is able to induce significant B cell and T cell responses to both H3N2 and H3N8 CIV. It is expected that effective treatment methods using constructs developed according to the methods in this Example (and generally in this disclosure) will result in a vaccine that elicits an effective immune response within seven to fourteen days of an initial (and possibly single/only) administration in terms of a significant reduction in viral shedding, reduction in viremia, and/or reduction in severity of CIV-related symptoms (e.g., cough, runny nose, fever, lethargy, eye discharge, and reduced appetite).

Expression Library Immunization studies will be conducted in vitro with infected cells and in healthy dogs. B and T cell responses to vaccine antigens in each of the pooled study groups will be evaluated and confirmed using ELISA, Flow Cytometry, and/or ELISpot. The results of this work will lead to the identification of clinically relevant antigens (CRAs) relevant to CIV. Nucleotide sequences encoding CRAs or variants of such CRAs (e.g., deglycosylation variants) will be generated and inserted into gD-antigen fusion protein constructs (e.g., gD1 seq 1 and gD1 seq 2 constructs, in which CIV CRA-encoding sequences will be inserted).

Also, a number of known CIV antigens can be used to generate putative gD-antigen fusion protein-encoding nucleic acid constructs. Candidate CIV antigens for use in such testing methods include: (a) Hemagglutinin A surface antigen or portions thereof (e.g., the conserved stalk—H3N2 HA stalk domain); (b) Ion channel protein M2 or portions thereof (e.g., domain M2 or domain M2e); (c) Nucleoprotein (NP); (d) M1 (matrix protein 1); (d) Neuraminidase (N1, N2); or (e) combinations of any/all thereof.

Also, putative antigens expected to be associated with “unnatural immunity” (Scorza 2016; Nabel et al. 2010, supra) (i.e., to induce antibody and cellular immunity to viral antigens that are not naturally or effectively recognized by the immune system but that can provide protection when presented to the immune system in a vaccine) are selected and related sequences incorporated into selected, suitable delivery systems.

Adenoviral vectors and/or DNA plasmids (associated with CaPNPs) will be generated comprising these sequences, EAT-2, and these gD-antigen fusion protein-encoding sequences plus EAT-2. These plasmids (and suitable control plasmids) will be first used in suitable safety/immunogenicity studies then in an immunization/challenge study in healthy dogs. An exemplary set of challenge study plasmid constructs could include: (a) Control; (b) hEAT-2; (c) hEAT-2+ gD1 seq 1: CRA: gD1 seq 2; and (d) gD1 seq 1: CRA: gD1 seq 2.

A variety of CRAs are expected to be identified by ELI, such that the number of CRA-encoding constructs is likely to be ≥1 in instances.

AOA, known Ag(s) expected to be effective in expression constructs of the invention can be directly generated and similarly tested. Candidate CIV antigens that can be include in such test constructs can include: The CIV HA stalk domain (HA-SD); CIV NP; CIV M1; and CIV M2.

For example, adenoviral vectors and/or DNA plasmids expressing HA Stalk Domain (HA-SD) fused to one or more of NP, M1, and M2 proteins can be tested in the following combinations and analyzed for B and T cell responses: (1) AdC68-HA-SD/NP/M1/M2; (2) AdC68-gD-HA-SD/NP/M1/M2; (3) CaPNP-gD-HA-SD/NP/M1/M2; (4) gD-HA-SD/NP/M1/M2; and Nobivac® Canine Flu Bivalent Vaccine (as a control/comparator).

All plasmids will be confirmed by DNA sequencing before transfecting into HEK293 and Cf2Th cells. Expression of individual proteins will be confirmed by western blotting using commercially available antigen-specific monoclonal or polyclonal antibodies.

Adenovirus shuttle plasmid and DNA vaccine plasmids with multi-gene co-expression using 2A cleavage sequences can be generated and analysis of constructs for antigen expression performed using methods discussed above. Plasmids can be used for viral vector delivery and plasmids are suitable for DNA vaccination with nanoparticles at the same time because the cloning strategy is similar. In vitro studies and vaccination with plasmid DNA delivered in nanoparticles and in a replication-defective adenoviral vector can be formed to conduct direct comparison of immune responses to the two different delivery systems. Adenoviral vector constructs with and without gD allow for evaluation of gD responses DNA sequencing before transfection of HEK293 and Cf2Th cells and detection of transgene proteins by western blot analysis will confirm that all plasmids are suitable for virus vector preparation and incorporation into particles. gD fusion antigens will be separated by cleavable spacers so that some antigens will be released into the circulation for exposure to B cells. Antigen protein expression will be analyzed with Western blots. If antigens are not expressed or not properly cleaved, the positions of the NP, M1, and M2 genes in relation to each other will be reordered to optimize expression of all 4 proteins.

E1/E3 deleted simian adenovirus Adc68, HuAd5 or other adenoviral strain not endemic in dogs can be used for adenoviral vector delivery systems. The well-established procedure of homologous recombination to insert shuttle plasmids into the backbone adenoviral plasmid will be used. HEK293 cells expressing HAd5 E1 gene products will be used for generation of replication deficient Ad-CIV101 recombinant virus. The steps involved are: 1) transfect the shuttle plasmid into the 293A Cell Line to produce an adenoviral stock, 2) amplify the adenoviral stock on 10×T75 flasks, 3) purify the virus, 4) titer the virus, 5) use the amplified adenoviral stock to transduce HEK293 and Cf2Th cells and analyze by western bot of transient expression of antigens, and 6) use purified and expression-confirmed virus for mouse immunogenicity studies. DNA plasmids will be generated and formulated with CaPNP before testing (e.g., CaPNP-gD-HA-SD/M1/M2).

The safety and immunogenicity of each of the candidate vaccines and commercial standard(s) will be assessed by administration to healthy dogs (e.g., 5 dogs per group), receiving 10 to 100 micrograms of plasmid DNA via mucosal or subcutaneous administration of CaPNP-gD-HA-SD/NP/M1/M2 or naked DNA gD-HA-SD/NP/M1/M2, optionally in combination with an EAT-2 expressing sequence or nucleic acid. Nucleic acid compositions will be delivered by mucosal or subcutaneous administration and 1 mL Nobivac® Canine Flu Bivalent Vaccine (killed H3N2 & H3N8 virus plus alum adjuvant; a conventional vaccine comparator) also is administered per product instructions.

Blood will be collected from treated animals before vaccination and at 24, 48 and 168 hours (7 days), 14, 21 and 42 days. Assays performed after vaccination can include CBC, plasma chemistry: baseline and at 24, 48, and 168 hours (7 days). Health inspection by (a) veterinarian(s), including rectal temperature, can be performed at baseline, 1-, 3-, 6-, and 12-hours post-treatment; once daily thereafter; and clinical observations on days when a physical exam is conducted (Days 2 & 7.)

PBMC sampling from each group of 5 dogs can be taken at baseline, and 7, 14, 21 and 42 days after vaccination. Blood volume taken will be 10⁻¹⁵ ml. Blood collection procedures for T cell assays are conducted as follows: For each bleed/each dog, use a 2 BD Vacutainer® CPT™ Mononuclear Cell Preparation Tube—Sodium Heparin (16 ×125 mm/8 mL) from BD Bioscience) to collect 10⁻¹⁵ ml blood and process according to manufacturers' instructions. Because there can be a circadian rhythm to some immune responses, blood samples are be collected at approximately the same hour each sampling day throughout the study.

B cell responses in dogs can be assessed through virus neutralization and ELISA. For example, antibodies to NP, M2, or H3 (polyclonal) can be purchased currently, or it is possible to generate polyclonal antibodies, for example to HA stalk. Enzyme-linked immunosorbent assay (ELISA) can be performed for antibodies to CIV antigens using sera from vaccinated and naive mice/dogs on plates coated with purified CIV antigens.

Intracellular cytokine staining can be performed as briefly described here: PBMCs will be tested by ELISpot assays for production of interferon-gamma in response to the influenza antigen-specific peptides present in the vaccine. CIV antigen-specific CD8+ and CD4+ T cell analyses will be conducted on selected cryopreserved PBMCs. All samples from each dog will be analyzed in parallel. Cells will be stained with a live cell stain, antibodies to CD3, CD4 and CD8 and intracellular interferon-gamma and TNF-alpha. A goal of this experiment is to determine if frequencies of T cells to influenza-associated antigens increase after vaccination. It is expected that days 7-14 will show peak IRs, while week 6 responses are expected to represent steady state IRs.

The Phase I study will be followed by advancing one or more CIV antigen constructs to a Phase I challenge study in dogs. Through application of such methods, one or more candidates for pivotal trials and further development into approved treatments for CIV are expected to be developed.

### IX. Example 8—EHV Treatment

This Example outlines the application of principles, compositions, and methods of this invention to develop candidate vaccines leading to an effective vaccine that elicits effective, long-lasting protective immunity to equine herpes virus (EHV). It is specifically expected that identified effective vaccines developed by such methods will make significant improvements in terms of EHV-related myeloencephalopathy and abortion rates and both prevent infection and reactivation from latency.

The EHV-1 genome consists of 80 ORFs (Paillot R. et al. 2008) that separate into four categories (Allen et al 2004):
1. nucleocapsid and tegument structural proteins;
2. transmembrane/envelope glycoproteins;
3. regulatory/transcription activation proteins;
4. DNA replication, viral packaging, morphogenesis, and egress proteins.

Putative CRAs can be identified by Expression Library Immunization (ELI) as discussed above and/or mass spectrometry (MS) using known methods. Alternatively, known EHV candidate antigens to be directly tested in methods similar to those described in the preceding Examples can include: (a) IE (ICP4, ORF 64 intermediate early) protein; (b) ORF-12 tegument protein α-TIF; (c) ICP0; (d) gB; (e) gD; (f) gH; (g) gL; (h) gK; (i) gC; (j) gE; (k) gG; (l) gI; (m) gM; (n) sp300; (o) gE/gI; (p) gM; (q) gp300 (gp2); (r) UL27 (gB); (s) UL29 (major DNA binding protein); (t) UL39 (ribonucleoside reductase R1); (u) ICP4 (IE protein); (v) ICP0 (ubiquitin E3 ligase); (w) ICP22 (intermediate early infectious cell protein 22); (x) ICP27 (intermediate early infectious cell protein 27); (y) VP22 (tegument); (z) VP26 (small capsid protein); (aa) TK (thymidine kinase); and (bb) ICP10 (intermediate early infectious cell protein 10).

In one variant, antigens will be selected to elicit “unnatural immunity” (Scorza 2016; Nabel et al. 2010), that is, to induce antibody and cellular immunity to viral antigens that are not naturally or effectively recognized by the immune system, but are capable of providing effective, broad-spectrum protection when presented to the immune system.

Confirmation of antigen expression from constructs will be performed in human kidney HEK293 and equine monocytes or dendritic cells. All plasmids will be confirmed by DNA sequencing before transfecting into HEK293 and equine cells. Expression of individual proteins will be confirmed by western blotting using antigen-specific monoclonal or polyclonal antibodies. PBMC from naturally EHV infected horses can be used to probe CTL recognition of cultured cells transfected with antigen-bearing constructs. Endotoxin-free plasmid megapreps will be prepared and plasmids formulated with at least one vaccine candidate for pilot testing. Naïve horses will be vaccinated subcutaneously or mucosally with constructs to evaluate safety and CTL response to the vaccine candidates. This step will confirm safety and characterize the cellular immune response to vaccine antigens by ELISpot and flow cytometry. T cell responses to vaccine antigens in vitro using PBMC from naturally infected horses will also be analyzed. The results of these studies will be used to select candidates for phase 2 challenge studies and to develop effective medicines.

### X. Example 9—Coronavirus Constructs

This Example demonstrates the application of similar approaches to those described in the preceding Examples to Coronavirus SARS COV2.

Vaccine compositions comprising gD-antigen fusion protein constructs comprising SARS COV2 antigens are prepared comprising either a single plasmid (comprising both gD-antigen fusion protein-encoding sequences and an EAT-2 polypeptide coding sequence) or two plasmids (one plasmid containing gD: SARS COV2 antigen-encoding sequence and the other plasmid comprising an EAT-2-coding sequence). Constructs generated for performing these experiments will either include (1) constructs encoding only gD sequences that do not effectively bind HVEM or (2) constructs that separately contain both HVEM-binding and non-effective-HVEM-binding gD sequences. SARS COV2 antigens may only be contained in the C-terminus of the gD-antigen fusion protein, internally to two gD domains, or both. EAT-2 sequences will comprise human EAT-2 (hEAT-2) or a functional fragment thereof or murine EAT-2 (mEAT-2) or a function fragment thereof or both types of sequences will be included among different constructs.

SARS COV2 antigens contained in the fusion protein(s) expressed by constructs used in the study will include SARS COV2 surface glycoprotein (S), Nucleocapsid (N), Membrane glycoprotein (M) sequences, or combinations thereof. Variants can be generated and tested (either as the only test candidates or in comparison to other candidates) by substitutions introduced in one, several, or all of the N—X—S sequences or N-X-T sequences present in such antigen amino acid sequences. An example of a source of candidate sequences and virus that can be used in testing of vaccine candidate constructs is provided at NCBI Reference Sequence: NC_045512.2 (severe acute respiratory syndrome coronavirus 2 isolate Wuhan-Hu-1, complete genome).

An example of a SARS COV2 Ag, a COV2 S sequence (SEQ ID NO: 14) (Accession YP_009724390.1) includes several potential sites for making GSRV(s) (e.g., AARs 17-19, 61-63, 74-76, 122-124, 149-151, 165-167, 234-236, 282-284, 331-333, 343-345, 603-605, 616-618, 657-659, 709-711, etc. that are modified in SARS COV2 S AgV(s).

Another example of a SARS COV2 Ag, a SARS COV2 N sequence (SEQ ID NO: 15) (Accession YP_009724397.2 nucleocapsid phosphoprotein) similarly includes several possible GSRV site(s) at residues 47-49, 77-79, 192-194, 196-198, and 269-271. NS(s) encoding such GSRV(s) are generated.

Still a further example of a SARS Ag, a COV2 M sequence (SEQ ID NO: 16) (Accession YP_009724393.1 membrane glycoprotein) includes a possible GSRV site at residues 5-7.

Using the above-described elements, constructs comprising SEQ ID Nos 15-17 or GSRV(s) of any or all thereof will be developed and evaluated first in nonclinical settings and then in human clinical trials.

### Listing of Exemplary Aspects of the Invention

The following is a non-limiting list of exemplary aspects of the invention, intended to further illustrate embodiments in a summary form.

In aspects, the invention provides CEPESCs/compositions comprising EA(s) of NAM(s) comprising NS(s) comprising (1) AgES(s); and (2) one or more of (a) gDPES(s); (b) ICITMES(s) (e.g., ITICITMES(s)); ICSTAPES(s) (e.g., ITICSTAPES(s)); or CPSTAPES(s) (e.g., EAT-2 PPT ES(s)); (c) ITS(s) (e.g., PTPS(s), such as polyUB(s)); (d) EEI(s) (e.g., a CMV Intron A EEI); the NS(s) optionally associated with TFA(s), such as CaPNP(s) and the NS(s) optionally being in NAVs, such as plasmid(s) or RNA(s).

In aspects, Ag(s) in the EP(s) of composition(s) comprise multiple Ag(s), optionally expressed in PE(s)/FP(s), FP(s) optionally comprising SCS(s), MSL(s), FL(s), MSFL(s), etc.; Ag(s) optionally comprising CD4TCE(s) and CD8TCE(s) (which in aspects result in DOS CD4 TC IR(s), CD8 TC IR(s), and BC IR(s)); Ag(s) optionally comprising unnatural immunity Ag(s), AgV(s) from which decoy epitope(s) are removed, subdominant epitope(s), cryptic epitope(s), etc.; TCE(s) associated with flanking sequence(s); other editope(s); or Ag(s) optionally comprising DIV(s) (which may comprise GSRV(s)); or any combination thereof. In cases, Ag(s) comprising the BCE(s) are AgV(s) comprising a reduced number of BCEs with respect to its WTC. In aspects, EP(s) comprise DIV(s) and the CEPESC produces DOS reduced cytokine storm adverse event(s) (CSAE(s)) or VAERD in TR(s). Ag(s) can comprise dominant epitope(s), subdominant epitope(s), or both. Ag(s) can be anti-cancer Ag(s) (CAg(s)), anti-viral Ag(s), or other DCA-associated Ag(s).

In cases, compositions also comprise gDPES(s) encoding gDP(s) that (i) lack a gD TMD, (ii) exhibit DOS reduced affinity for HVEM than HSV-1 gD, or (iii) both (i) and (ii), and further optionally comprise a gDSS. In aspects, gDP(s) comprise gDAgFP(s). In cases, Ag(s) are positioned downstream of gDS(s) in gDAgFP(s).

In cases, compositions also comprise ES(s) encoding PCIs. In aspects, gDS(s) have CI functions. In aspects, EP(s) include non-gD PCI(s) (e.g., a PCT that modulates a CD112R or PD-1 checkpoint pathway). In aspects, PCI(s) are non-Ab PPT(s), such as multimeric trap PPT(s).

In cases, compositions also comprise EP(s) encoding non-antibiotic selection marker(s) (NASM(s)), e.g., a triclosan resistance marker.

In cases, compositions also comprise targeting sequence(s), e.g., non-gD immune cell receptor targeting sequence(s), such as DEC-205-TS(s). In aspects, compositions also include NS(s) encoding endosome TS(s), lysosome TS(s), ERTPS(s), etc., or PTPS(s), such as polyUb(s).

In cases, NS(s) encoding any one or more of the above-described EP(s) are contained in ≥2 NAM(s) in the composition (e.g., in aspects 1 NAM encodes a gDPAgFP and ≥1 other NAM encodes (a) Ag(s), (b) EAT-2 PPT(s), (c) NGDPCI(s), or (d) a combination of any or all thereof).

In aspect, NS(s) further encode anti-DCA/pro-IR cytokine(s).

In aspects, EP(s) include MgD(s), e.g., gDP(s) with enhanced nectin-1 binding over HSV-1 gD; no detectable HVEM binding; or both.

In cases, gDP(s) comprise sequence(s) that are RVRHRSIOI to SEQ ID NOs: 78, 65, 66, 68, any of SEQ ID NOs:70-74, SEQ ID NOs:79 or 82, or a suitable combination thereof. In cases, gDP(s) do not comprise any sequence that exhibits more than about 60% or 65% amino acid sequence identity to SEQ ID NOs: 77, 76, 75, or 81. In aspects, gDP(s) lack any functional gD TMD. In cases, gDAgFP(s) comprise (a) a first gDS (gDS1) that is RVRHRSIOI to AAs 23-244 of HSV-1 gD & (b) a second gDS (gDS2) that is RVRHRSIOI to AAs 244-340 of HSV-1 gD, and (c) Ag(s) positioned between gDS1 and gDS2; Ag(s) positioned downstream of gDS2; or both.

In cases, gDP(s) comprise gDS(s) that detectably bind HVEM under typical gD:HVEM binding conditions. In cases, gDP(s) do not bind HVEM. In cases, gDP(s) comprise a gDSS, a gD PFD, or both.

In cases, Ag(s) comprise, primarily comprise, GCO, SCO, consist essentially of, or consist of ASFV Ag(s) (e.g., 8-50 AARs of any one of SEQ ID NOs:23, 237, 27, 28; 238-423; an ASFV CP204 L ORF EP; an ASFV p32/p30 protein; ASFV ORF CP530R; ASFV ORF E183 L, ASFV ORF EP402R, ASFV ORF B646; ASFV p32, p54, pp62 or p72 proteins; or FF(s) or FV(s) of any thereof.

In cases, Ag(s) comprise, primarily comprise, is generally comprised of, is substantially comprised of, or is comprised of (CPCGCOSCOOCO) PCV Ag(s) (e.g., a PCV ORF1 sequence or an antigenic variant thereof; a PCV ORF2 sequence or a FV thereof; etc., such as Ag(s) of PCV ORFs 1 and 3, PCV ORFs 1 and 4, PCV ORFs 2 and 3, PCV ORFs 2 and 4, or PCV ORFs 3 and 4). In aspects, PCV Ag(s) comprise EP(s) of (a) a PCV ORF1, (b) a PCV ORF2, and (c) a PCR ORF3, a PCV ORF4, FFs or FVs of any thereof, or a combination thereof. In aspects, PCV Ag(s) comprise expression products of ≥1 of PCV ORF5, ORF6, ORF7, ORF8, ORF10, ORF11, and ORF12. In aspects, PCV Ag(s) comprise ≥8, ≥12, or ≥20 AAs of or that is RVRHROSI to any one or more of SEQ ID NOs: 24, 25, 143-154, 654-666. In aspects, PCV Ag(s) comprise ≥1 GSRAgV(s) (e.g., a PCV ORF2A143-145 sequence). In aspects, PCV Ag(s) comprise PCV-2d Ag(s), PCV-3 Ag(s), or both. In aspects, PCV Ag(s) comprise at least 8 AAs of SEQ ID NO:155, SEQ ID NO:156, or both. In aspects, EP(s) comprise PCV-3 Ag(s) encoded by a portion of SEQ ID NO: 157. In aspects, PCV-3 Ag(s) comprise GSRAgV(s), e.g., at least 8 AAs of SEQ ID NO:439, comprising residues 16-18 thereof; at least 8 AAs of SEQ ID NO:440 comprising residues 124-126 thereof; or a portion of SEQ ID NO:441 comprising AAs 16-126 thereof.

In aspects, Ag(s) CPCGCOSCOOCO PRRSV Ag(s), such as PRRSV ORF3 Ag(s) (e.g., PRRSV GP3 sequence(s)), PRRSV ORF1a Ag(s) (e.g., PRRSV nsp7 sequence(s); PRRSV ORF7 Ag(s) (e.g., PRRSV protein N sequence(s)). In aspects, PRRSV Ag(s) comprise PRRSV envelope glycoprotein GP2 (ORF2) Ag(s); envelope glycoprotein GP4 (ORF4) Ag(s); nucleocapsid protein ORF7 Ag(s); PRRSV ORF1a′ Ag(s), PRRSV ORF1b Ag(s), PRRSV ORF2a Ag(s), PRRSV ORF2b Ag(s), PRRSV ORF4 Ag(s), PRRSV ORF5 Ag(s), PRRSV ORF5a Ag(s), or PRRSV ORF6 Ag(s), etc. In aspects, Ag(s) include AAs 1-100 of a PRRSV ORF3 sequence; PRRSV NSP1α sequence(s) or NSP1β sequence(s); PRRSV NSP2 sequence(s); PRRSV NSP3 sequence(s); PRRSV NSP4 sequence(s), NSP5 sequence(s), NSP7a sequence(s), NSP7β sequence(s), NSP8 sequence(s), NSP9 sequence(s), NSP10 sequence(s), NSP11 sequence(s), NSP12 sequence(s), or a combination thereof. In cases, PRRSV Ag(s) comprise 1+ of SEQ ID NOs: 120-132 or FF/FV(s) thereof; ≥8, ≥12, ≥20 AAs of SEQ ID NO:11 or a FV thereof; or both. In aspects, PRRSV Ag(s) comprise GSRAgV(s). In aspects, PRRSV Ag(s) comprise ≥8, ≥12, ≥20 AAs of SEQ ID NOs:12 or 13 or a FV thereof. In aspects, PRRSV Ag(s) include a sequence of or VRHROSI to a sequence of 8-40 AARSs of one or more of SEQ ID NOs: 424-438.

In aspects, Ag(s) comprise, primarily comprise, GCO, SCO, or consist of (CPCGCOSCOOCO) EHV Ag(s). In aspects, EHV Ag(s) comprise EHV1 Ag(s). In aspects, EHV Ag(s) comprise EHV immediate early gene ICP4 epitope(s), EHV Tegument Protein α-TIF epitope(s) (e.g., ICP0, ICP22, and ICP2 epitopes); or both. In cases, EHV Ag(s) comprise sequence(s) of at least 8 AAs of one of SEQ ID NOs: 158-237. In cases, EHV Ag(s) comprise GSRAgV(s) comprising GSRV(s) in ≥1 of SEQ ID NOs: 159-160, 162-164, 166, 168-175, 178-181, 185, 187, 189-190, 194, 196, 198-200, 202-203, 206-207, 210214, 218, 221, 224-225, 227-231, 233, and 234. In facets, EHV Ag(s) include ≥8 AAs of one of SEQ ID NOs: 442-449.

In aspects, Ag(s) CPCGCOSCOOCO) coronavirus (COV) Ag(s). In aspects, COV Ag(s) CPCGCOSCO or consist of SARS-COV2 Ag(s) (e.g., SARS-COV2 S protein Ag(s) or HR or SI FV(s) thereof). In cases, COV Ag(s) comprise ≥8, ≥12, or 20 AAs of SEQ ID NO: 14 or a FV thereof. In cases, COV Ag(s) comprise ≥8, ≥12, ≥20 AAs a sequence according to SEQ ID NO: 20 or SEQ ID NO:744. In aspects, COV Ag(s) comprise GSRV(s) (e.g., a substitution in a glycosylation site of SEQ ID NO: 14). In aspects, COV Ag(s) comprise a sequence of a SARS-COV2 N protein or a FV thereof. In aspects, COV Ag(s) comprise ≥8, ≥12, or ≥20 AAs of SEQ ID NO: 15 or a FV thereof. In aspects, COV Ag(s) comprise sequence(s) of 8, ≥12, or ≥20 AAs of the formula of SEQ ID NO: 21. In aspects, COV Ag(s) comprise ≥8, ≥12, or 20 AAs of SEQ ID NO: 21 and comprise ≥1 substitution of a corresponding Asn residue in SEQ ID NO: 15. In aspects, COV Ag(s) include SARS-COV2 M protein sequence(s) or FV(s). In aspects, COV Ag(s) comprise 8, ≥12, or ≥20 AAs of SEQ ID NO: 16 or a HR or SI FV thereof. In cases, COV Ag(s) comprise 8, 12, or 20 AAs of SEQ ID NO: 22 or a HR or SI FV thereof. In facets, COV Ag(s) include SARS-COV2 E sequence(s) or FV(s) thereof.

In aspects, cancer Ag(s) in EP(s) PCGCOSCO or CO transitional cell carcinoma Ag(s), bladder cancer Ag(s), or both. In aspects, cancer Ag(s) comprise B-RAF Ag(s). In aspects, cancer Ag(s) comprise ≥8, ≥12, or ≥20 AAs of one of SEQ ID NOs: 450-453 or an FV. In cases, cancer Ag(s) comprise MAGE-3 Ag(s) (e.g., ≥8, ≥12, or ≥20 AAs of SEQ ID NO:454 or a FV thereof). In cases, cancer Ag(s) comprise NY-ESO-1 Ag(s) (e.g., ≥8, ≥12, or ≥≥20 AAs of SEQ ID NO:455 or an FV). In cases, cancer Ag(s) include Her-2 Ag(s) (e.g., ≥8, ≥12, or ≥20 AAs of SEQ ID NO:456 or a FV).

In aspects, any of the above-described composition(s) is a dried composition that is reconstitute-able and shelf stable for a period of at least one year at room temperature (or ordinary environmental temperatures) and typical humidity conditions.

In aspects, the invention provides a composition according to any one or more of the preceding paragraphs of this section and means for administering the composition to a subject.

In cases, the invention provides methods of inducing DOS IR(s) in TR(s) comprising delivering EA(s) of CEPESC(s) according to any one or more of the above-provided paragraphs to the TR(s) ≥1 times (e.g., ≥2 times). In aspects, the method treats or prevents a DCA-associated disease. In aspects, the method treats or prevents a pathogen that is a leaky vaccine associated pathogen. In aspects, TR(s) is/are non-HVEM-expressing TR(s) (e.g., swine). In aspects, methods include monitoring the TR(s) for (i) inducement of CSAE(s) in response to delivery of the 1^{st} composition, (ii) inducement of VAERD in response to delivery of the 1^{st} composition, (iii) inducement of an immunogenic adverse reaction to the ₁St composition, (iv) development of immunological tolerance to Ag(s) of the 1^{st} composition, or (v) a combination of any or all of (i)-(iv); and (c) if any of the conditions in (b) is detected (or detected in an amount meeting standard(s)) adjusting the treatment regimen by changing the frequency of CEPESC delivery, dosage of CEPESC(s), composition of subsequently delivered CEPESC(s), or delivery of other compounds/performance of other step(s).

In another aspect, the invention provides methods of developing medicaments comprising (a) identifying PCRA(s), (b) delivering the PCRA(s) to IC(s) of TR(s), (c) assessing IR(s) induced by the PCRA(s), (d) selecting CRA(s) that induce IR(s) at or above a standard in the ICs; (e) obtaining NSs encoding the CRA(s); and (f) developing a construct comprising the CRAES(s) and optionally other components of the compositions described above (e.g., PTPS(s), EEI(s), gDSES(s), PCI encoding sequence(s), and the like). Additional similar and related aspects are described in US20200325182.

In another aspect, the invention provides composition(s) such as those described in this section, wherein such composition(s) comprise only naturally occurring sequences.

In another aspect, the invention provides composition(s) such as those described in this section, wherein such composition(s) do not comprise one or more, e.g., collections of, non-native T-cell epitope(s) (epitope(s) that do not correspond to epitope(s) of a target pathogen or cancer/DCA, such as synthetic epitope(s), non-natural variations/mutations of epitope(s), etc.) or non-natural epitope(s) (epitope(s) not found endogenously/naturally (natively) in any target pathogen/DCA or related pathogen DCA). In another aspect, the invention provides composition(s) such as those described in this section or elsewhere herein, and methods of making the same, wherein such composition(s) are not generated using a process or method comprising epitope prediction or lack any epitope that is identified only through epitope prediction (rather than being natively present). In another aspect, the invention provides composition(s) such as those described in this section, wherein such composition(s) mostly, generally only, substantially only, or only comprise sequence(s) encoding antigen(s) that are characterizable as partial or complete contiguous sequences of a native or natural protein/antigen, such as antigen(s) that are naturally expressed in typical target DCA(s) or related DCA(s) thereto.

1. A composition suitable for pharmaceutical or veterinary use comprising:
(1) an effective amount of one or more DNA or RNA nucleic acid molecules, the one or more nucleic acid molecules comprising one or more nucleotide sequences that encode (a) one or more antigens, each of the one or more antigens being associated with a disease-causing agent and (b) one or more innate trained immunity immunomodulators, and
(2) an effective amount of either (a) calcium phosphate nanoparticles associated with the one or more nucleic acid molecules and that detectably enhance cellular uptake of the one or more nucleic acid molecules, immunogenicity of the composition, or both, or (b) a nonpathogenic bacterial vector that contains the one or more nucleic acid molecules and detectably promotes delivery of the one or more nucleic acid molecules.

2. The composition of claim 1, wherein the one or more innate trained immunity immunomodulators comprise one or more immunomodulatory signal transducing activator protein amino acid sequences.

3. The composition of claim 2, wherein the one or more immunomodulatory signal transducing activator protein amino acid sequences modulate a signaling lymphocyte activation molecule receptor.

4. The composition of claim 3, wherein the one or more immunomodulatory signal transducing activator protein amino acid sequences are Ewing's sarcoma-associated transcript-2 sequences.

5. The composition of claim 1, wherein the one or more nucleotide sequences encode at least one checkpoint inhibitor.

6. The composition of claim 5, wherein the at least one checkpoint inhibitor does not comprise an antibody, antibody fragment, or antibody fusion protein.

7. The composition of claim 2, wherein the one or more nucleotide sequences encode a fusion protein comprising at least one of the one or more antigens and one or more polyubiquitin sequences.

8. The composition of claim 7, wherein at least one of the one or more antigens comprises at least one glycosylation site removal variation.

9. The composition of claim 2, wherein at least one of the one or more antigens is expressed as a fusion protein comprising an immune cell receptor targeting sequence.

10. The composition of claim 2, wherein at least one of the one or more nucleic acid molecules comprises an expression-enhancing intron that is operatively linked to an antigen-encoding nucleotide sequence.

11. The composition of claim 2, wherein the one or more nucleotide sequences encode two or more antigens, a plurality of the two or more antigens being expressed in a fusion protein and the fusion protein comprising (a) one or more linkers that are mid-sized linkers, flexible linkers, or mid-sized and flexible linkers, wherein the one or more linkers link at least two antigens in the plurality of the two or more antigens and/or (b) one or more self-cleavage site(s) that are positioned between at least two antigens in the plurality of the two or more antigens.

12. The composition of claim 2, wherein the one or more nucleotide sequences comprise a sequence encoding a triclosan selection sequence.

13. The composition of claim 2, wherein the one or more antigens comprise one or more Porcine Circovirus antigens, Porcine Reproductive and Respiratory Syndrome Virus antigens, African Swine Fever Virus antigens, Porcine Coronavirus antigens, Porcine Epidemic Diarrhea Virus antigens, Porcine Rotavirus antigens, Porcine Sapovirus antigens, Transmissible Gastroenteritis Virus antigens, Porcine Deltavirus antigens, or a combination of any or all thereof.

14. The composition of claim 2, wherein the composition comprises at least two nucleic acid molecules, wherein at least one of the at least two nucleic acid molecules comprise at least one nucleotide sequence that is not contained in at least one other nucleic acid molecule in the composition, at least two of the at least two nucleic acid molecules in the composition comprise different nucleotide sequences that encode different expression products, at least one of the at least two nucleic acid molecules comprises a nucleotide sequence encoding one or more antigens, and at least one of the at least two nucleic acid molecules does not encode any antigens that induce immune responses against the disease-causing agent.

15. The composition of claim 2, wherein the one or more antigens comprise one or more coronavirus antigens from a coronavirus that infects a species that is known to express Herpes Virus Entry Mediator or a homolog thereof.

16. The composition of claim 2, wherein the one or more antigens comprise one or more cancer antigens.

17. The composition of claim 5, wherein the one or more antigens comprise one or more cancer antigens.

18. The composition of 14, wherein the one or more antigens comprise one or more cancer antigens.

19. The composition of claim 2, wherein the one or more antigens consist of antigens that are typically naturally expressed in a target disease-causing agent.

20. The composition of claim 14, wherein the one or more antigens consist of antigens that are typically naturally expressed in a target disease-causing agent.


SEQUENCE LISTING (755 sequences)

SEQ ID NO: 1 - protein, 75 aa, Homo sapiens
MQIFVKTLTGKTITLEVEPSDTIENVKAKIQDKEGIPPDQQRLIFAGKQLEDGRTLSDYNIQKESTLHLVLRLRG

SEQ ID NO: 2 - DNA, 6153 bp, synthetic construct
  misc_feature 1..6153: note = SYNTHETIC CONSTRUCT DERIVED FROM HUMAN THOUGHT
  source 1..6153: mol_type = other DNA
cctgcaggtcgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccaagcttggatccgctagcgccaccatgcagatcttcgtgaagaccctgaccggcaagaccatcaccctggaggtggagcccagtgacaccatcgagaatgtgaaggccaagatccaggataaggagggcattccccccgaccagcagaggctcatctttgcaggcaagcagctggaggatggccgcactctttctgattacaacatccagaaagagtccaccctccatctggttctgcgtctgaggggtgcggccgcagattttattttaaatatatccatgaaaatggaggtcatcttcaaaacggatttaagatcatcttcacaagttgtgtttcatgcgggtagcctgtataattggttttctgttgagattatcaatagcggtagaattgttacgaccgctataaaaacattgcttagtactgttaagtatgatattgtgaaatctgctcgtatatatgcagggcaagggtatactgaacatcaggctcaagaagaatggaatatgattctgcatgtgctgtttgaagaggagacggaatcctcagcatcttcggagaacattcatgaaaaaaatgataatgaaaccaatgaatgcacatcctcctttgaaacgttgtttgagcaagagccctcatcggaggtacctaaagactccaagctgtatatgcttgcacaaaagactgtgcaacatattgaacaatatggaaaggcacctgattttaacaaggttattagagcacataattttattcaaaccatttatggaacccctctaaaggaagaagaaaaagaggtggtaagactcatggttattaaacttttaaaaaaaataagcttttatctcacctacattaccggttccggcgagggcaggggaagtcttctaacatgcggggacgtggaggaaaatcccggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgaggtttaaacgaattctgcagatatccagcacagtggtcgaggggcccggagatctacgtatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgacagctcgactctagaattgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattaattaattatttcagttcgagttcgttcattgcagcaatgctgaaaccgccgtcaacgtggaccacttcaccggagataccggcagagagatcggagcacaggaatgccgcagagttacccacatcttcaatagtaacggtacggcgaatcggggtaacggcttcgcaatgagccagcattttgcggaagtctttgataccggaggccgccagagtacggatcggaccagcagagatggcgttaacacgcacaccttccggacccatcgcgttcgccatatagcgcacgttcgcttccagagacgcttttgccagacccataacgttgtagttcgggatagcgcgctcagcgccaaggtaggaaagggtcagcagggcagaacccggattcagcatggagcggcaagcttttgccattgcaacgaagctgtaggagctgatgtcgtgggcaattttgaagccttcacgggtaacggcgttaacatagtcaccatccagctgatcgccaggtgcaaaaccaatagagtgtacgaaaccgtcaaatttcggccaaactttccccagttcagcgaacatggtgtcgatgctggcatcttctgcaacatcgcactgcagaacgatgtcagaacccaattgagcggcaaattcttctacgcggcctttcagtttgtcgttctggtaggtgaatgccagttcagctccttcgcggtgcatcgcctgagcgataccgtaggcgatggatagtttgctggcaacgccggttaccagaatgcgcttaccggaaagaaaacccatagctttaatccttattgttgatgcttgttgtgcctgaaaatcaggcgattcgtcgttttagtaaacagtacgaacagataaacggttattataatcaacctggctgtgagtagctatagttgccaggtccgaccggagcaggctgcggcagggggggcgcttttcccctcaccctaaccctctccccagaggggcgaggggaccgtattgtgcaaatattgttaccccagcaacaaacaggctcatacagcccctaaccctttcatggcgatggctgggacggttcagaccttgccgaatattctccagcacaatattattgaagcatttatcagggttattgtcggcgcgcctcagagattttgagacacaacgtggctttccccccccccccggcatg

SEQ ID NO: 3 - DNA, 6223 bp, synthetic construct
  misc_feature 1..6223: note = SYNTHETIC CONSTRUCT DERIVED FROM HUMAN THOUGHT
  source 1..6223: mol_type = other DNA
cctgcaggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaaagcttggatccgctagcgccaccatgcagatcttcgtgaagaccctgaccggcaagaccatcaccctggaggtggagcccagtgacaccatcgagaatgtgaaggccaagatccaggataaggagggcattccccccgaccagcagaggctcatctttgcaggcaagcagctggaggatggccgcactctttctgattacaacatccagaaagagtccaccctccatctggttctgcgtctgaggggtgcggccgcagattttattttaaatatatccatgaaaatggaggtcatcttcaaaacggatttaagatcatcttcacaagttgtgtttcatgcgggtagcctgtataattggttttctgttgagattatcaatagcggtagaattgttacgaccgctataaaaacattgcttagtactgttaagtatgatattgtgaaatctgctcgtatatatgcagggcaagggtatactgaacatcaggctcaagaagaatggaatatgattctgcatgtgctgtttgaagaggagacggaatcctcagcatcttcggagaacattcatgaaaaaaatgataatgaaaccaatgaatgcacatcctcctttgaaacgttgtttgagcaagagccctcatcggaggtacctaaagactccaagctgtatatgcttgcacaaaagactgtgcaacatattgaacaatatggaaaggcacctgattttaacaaggttattagagcacataattttattcaaaccatttatggaacccctctaaaggaagaagaaaaagaggtggtaagactcatggttattaaacttttaaaaaaaataagcttttatctcacctacattaccggttccggcgagggcaggggaagtcttctaacatgcggggacgtggaggaaaatcccggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgaggtttaaacgaattctgcagatatccagcacagtggtcgaggggcccggagatctacgtatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgacagctcgactctagaattgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattaattaattatttcagttcgagttcgttcattgcagcaatgctgaaaccgccgtcaacgtggaccacttcaccggagataccggcagagagatcggagcacaggaatgccgcagagttacccacatcttcaatagtaacggtacggcgaatcggggtaacggcttcgcaatgagccagcattttgcggaagtctttgataccggaggccgccagagtacggatcggaccagcagagatggcgttaacacgcacaccttccggacccatcgcgttcgccatatagcgcacgttcgcttccagagacgcttttgccagacccataacgttgtagttcgggatagcgcgctcagcgccaaggtaggaaagggtcagcagggcagaacccggattcagcatggagcggcaagcttttgccattgcaacgaagctgtaggagctgatgtcgtgggcaattttgaagccttcacgggtaacggcgttaacatagtcaccatccagctgatcgccaggtgcaaaaccaatagagtgtacgaaaccgtcaaatttcggccaaactttccccagttcagcgaacatggtgtcgatgctggcatcttctgcaacatcgcactgcagaacgatgtcagaacccaattgagcggcaaattcttctacgcggcctttcagtttgtcgttctggtaggtgaatgccagttcagctccttcgcggtgcatcgcctgagcgataccgtaggcgatggatagtttgctggcaacgccggttaccagaatgcgcttaccggaaagaaaacccatagctttaatccttattgttgatgcttgttgtgcctgaaaatcaggcgattcgtcgttttagtaaacagtacgaacagataaacggttattataatcaacctggctgtgagtagctatagttgccaggtccgaccggagcaggctgcggcagggggggcgcttttcccctcaccctaaccctctccccagaggggcgaggggaccgtattgtgcaaatattgttaccccagcaacaaacaggctcatacagcccctaaccctttcatggcgatggctgggacggttcagaccttgccgaatattctccagcacaatattattgaagcatttatcagggttattgtcggcgcgcctcagagattttgagacacaacgtggctttccccccccccccggcatg

SEQ ID NO: 4 - protein, 287 aa, Sus scrofa
MRICSIFTFMAYCCLLEAFTITVPKDMYEVEYGSNVTLECRFPVDKQLNLLALVVYWEMKDKKIIQFVNGEEDLNVQHSSYSQRAQLLKDQLFLGKASLQITDVKLQDAGVYCCLISYGGADYKRITLKVNAPYRKINQRMSLDPVTSEHELTCQAEGYPEAEVIWTSSDYQILSGKTTITSSQREEKLFNVTSTLRVNATTNEIFYCTFRRLGPEENSTAVLVIPEPYVDPARKRTHWVILGALLLLIAVTAIFCLKRNVRMMDVEKCGSRDMKSEKQNDTQFEET

SEQ ID NO: 5 - DNA, 513 bp, Cytomegalovirus sp.
  source 1..513: mol_type = genomic DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 6 - DNA, 204 bp, Cytomegalovirus sp.
  source 1..204: mol_type = genomic DNA
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagct

SEQ ID NO: 7 - DNA, 304 bp, Cytomegalovirus sp.
  source 1..304: mol_type = genomic DNA
gcgttacataacttacggtaaatggcccgcctcgtgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccggccccctattgacgtcaatgacggtaaatggcccgcctggcattatgcccagtacatgaccttacgggactttcctacttggcagtacatctacgtattagtcatcgctattaccat

SEQ ID NO: 8 - DNA, 825 bp, Cytomegalovirus sp.
  source 1..825: mol_type = genomic DNA
cgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcag

SEQ ID NO: 9 - DNA, 956 bp, Cytomegalovirus sp.
  source 1..956: mol_type = genomic DNA
tcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcc

SEQ ID NO: 10 - DNA, 215 bp, synthetic construct
  misc_feature 1..215: note = SYNTHETIC CONSTRUCT DERIVED FROM HUMAN THOUGHT
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 11 - protein, 254 aa, Porcine reproductive and respiratory syndrome virus
MVNSCTFLHIFLCCSFLYSFCCAVVAGSNTTYCFWFPLVRGNFSFELTVNYTVCPPCLTRQAATEIYEPGRSLWCRIGYDRCGEDDHDELGFMIPPGLSSEGHLTSVYAWLAFLSFSYTAQFHPEIFGIGNVSRVYVDIKHQLICAEHDGQNTTLPRHDNISAVFQTYYQHQVDGGNWFHLEWLRPFFSSWLVLNVSWFLRRSPANHVSVRVLQILRPTPPQRQALLSSKTSVALGIATRPLRRFAKSLSAVRR

SEQ ID NO: 12 - protein, 254 aa, synthetic construct
  REGION 1..254: note = SYNTHETIC CONSTRUCT DERIVED FROM HUMAN THOUGHT
MVNSCTFLHIFLCCSFLYSFCCAVVAGSDTTYCFWFPLVRGDFSFELTVDYTVCPPCLTRQAATEIYEPGRSLWCRIGYDRCGEDDHDELGFMIPPGLSSEGHLTSVYAWLAFLSFSYTAQFHPEIFGIGDVSRVYVDIKHQLICAEHDGQDTTLPRHDDISAVFQTYYQHQVDGGNWFHLEWLRPFFSSWLVLDVSWFLRRSPANHVSVRVLQILRPTPPQRQALLSSKTSVALGIATRPLRRFAKSLSAVRR

SEQ ID NO: 13 - protein, 100 aa, synthetic construct
  REGION 1..100: note = SYNTHETIC CONSTRUCT DERIVED FROM HUMAN THOUGHT
MVNSCTFLHIFLCCSFLYSFCCAVVAGSDTTYCFWFPLVRGDFSFELTVDYTVCPPCLTRQAATEIYEPGRSLWCRIGYDRCGEDDHDELGFMIPPGLSS

SEQ ID NO: 14 - protein, 1273 aa, Severe acute respiratory syndrome-related coronavirus (SARS-CoV-2)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 419 aa, Severe acute respiratory syndrome-related coronavirus (SARS-CoV-2)
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 16 - protein, 222 aa, Severe acute respiratory syndrome-related coronavirus (SARS-CoV-2)
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 17 - protein, 132 aa, Homo sapiens
MDLPYYHGRLTKQDCETLLLKEGVDGNFLLRDSESIPGVLCLCVSFKNIVYTYRIFREKHGYYRIQTAEGSPKQVFPSLKELISKFEKPNQGMVVHLLKPIKRTSPSLRWRGLKLELETFVNSNSDYVDVLP

SEQ ID NO: 18 - DNA, 3977 bp, Mus musculus
  source 1..3977: mol_type = genomic DNA
agaatctaattatggttacctacatgagcactagtgggcggttattagagcatctgctatttgttggtggtaacaacatcaaagaccacgacactccttcccccagaaaccgttaactgccaatagtcccttggggaggggagggctccatgagcctcactcctacccatgatgaaatgttgaaggcccaagcctggcagagcttctgcagcaaaccatgaaatgtgtcatgatgggaagatgtcatttcacagcacacctccttgtctcctagctcctacattctttccaccccttcttccacagtgtttcctgagccctgatacagacgccttgcttaaagctgagtactccatcactgattctccggactttgaccagctgaggacaagagagctaagactaccagagtgctttctgttcatctttactgactgacgacctgaaaacgctatggctcgtttctcaacgtacatcatctttacctctgtcctctgtcagctaacagtcacagcagcttctggaactctgaagaaggtggccggtgcccttgatggatctgtgacattcactctgaatatcactgaaataaaggttgactatgttgtatggacgttcaacacattctttcttgccatggtaaaaaaagacggcgttacatcacaaagtagtaacaaagaaaggatagtctttccagatggactctactccatgaagctcagccaattgaagaagaatgactctggagcctaccgtgcagagatttacagtacatcgagtcaggcttccttaatccaggagtatgtgctgcatgtctacaagcatttgtcaaggcccaaggtcaccatagatcggcaaagcaacaagaatggcacctgcgtaatcaatctgacatgttccacggatcaggacggggagaatgtaacctacagctggaaagctgtggggcagggggacaatcagtttcatgatggtgccaccctctccatcgcctggagatcaggagagaaagaccaggccttaacatgcatggccaggaatccagtcagcaacagtttctcaacccccgtctttccccagaagctctgtgaagatgctgccacggatctaacttcactcaggggcatcctatacatcctgtgcttctcagcagtgctcatcctatttgctgtcttgctgactatttttcatactatgtggataaagaaaggaaaaggatgtgaggaagacaagaagagagtggacaggcaccaggaaatgcccgacttgtgccctcacttagaggagaacgcagactatgacacaatcccttacacggaaaaaagaagaccagaagaagatgcaccaaacacattttattccactgtgcagatccccaaagtggtaaagagtcccagctccctgcctgcaaagccactcgtgccaaggtcattaagctttgaaaatgttatctagatgacagcactccgtcctctccagaaaaaaacaaaacaaaacaaaactgcaaaacaaacaaaacctcaccattctgagcagaaatgaaaacttctgtcaaagactgaatgtaaatgtctccccgccagacccacatgtttgcatccagacagagaagttcaggctcaagggctttcagggtacaatgactcttggggactgggatacagcccattcagacattctgactaagaatctctctatgcccatcctgtctccggtaatgatgaatgaggctgtgctcatagacagtggactaatttgtctgatgaagaaatttttgacaaagcacagcatttggctgtggcacagccatgttcgctgtttttattcacatttatagctgtatgtaaataatctcaatcattcagttcccagataaaagacacataaccttttatatttattataagcctttatagactagagctgggcagatatctaccctctaagctatttttgcctacttccctatcaataaccccaagatataatttgccatgttccatctaggctgctcttactccaattggcccagcccttatggccatattttcatgattccccaacacaatggtgtcttctctctccaccgtctctcctcatgaacctcctgcctcagatcccaagcctgggaaccaaaacctcacctccctctcttctgccctgctataggctgccgacatctttattcacccagtagttttaaattaaggagcagggaaacatggtatcactttgtgtaggtgaggattctctagtcccagagggcaaccagaccttggggaccagtgacaagtattacaatacatagcaacagaccaaacctcagcagaactcaaaacaaagaaaaggaacaggaagatgggaaaagcatgcaggttgatagacagcatgaagggttttcaaatgggtttttcagcttcgactgtaatggttaaagggatgagcccagactgtgccctaggacaagagaaaggggacactgagagctaaaccctgtctttcaaaaaccctattcaaaaaagctttcatttaaaagaaagaatgcctgaacacagagtgtcccacatcgcacatcagactgtggaagtgtttctctgggttctcacggttctcagaggctgctggagccctgacccaagcaggtatggctccatcctggagtgacagcagaactcagcaatgtccacacgtgcagaatgtaaactctggggagccactgaagcttgcactaaggttccaggggaggaaaaccaggcaacgtgtatcgaggttgggctttctgaatggacctgccttgtaagcctgcataaaactatgaaggcaaagcctaacttgcagtttagactccaggatgctggagctgcctaaaacataaaggatgctggagctgcccagaacataaagcatctactaagaaaattttcaggcactgaaaacacccgagaaagaggccacgtgtggttttggttggaaggtcatggaaacaggcctcctcaaacccattggaacatacatgatgataccatgtacccaagatgccagatacggagcttcaaggcttatacctctctgctgagttttctcctatctttggctctttctttgccacgccccatccctcccttttggaatgtgaatgtttcctctgccattgtgcattgaaagtatgtgagttttctattttttaagagctcacaactaggactttgccttacatctcataaaagacattggacattgaactgttatggctagggactcttgaagttagatggaatgtgttttgcattgtgtgatgaccgtaggcatattgagccaagacctgtgagccaagacgggagtgttatggattgagtcgaaaagattcaaggttgcgggcttgtgtggttcaacacttggtgttatttaagggtgctgtagaccctatagaagatagagtctgtctgcagacggtgagtctccaggaggcaggcctagaaagtgacagtcactcctggctctgggcaagccttctgctccacctcaggcagaatcctgtccaccatgttatgaagaagcagacattgcaaactccctccaacacaggccaagcccctaaagccgccatgcctctgatgacaatggattataacctctagaactcaaaatcgaaagcaggcccttcccctgtaagttgctcctgccaggattttggcagtgtggtagctgataccacttccttccagaataagctattctcagcctctttggactctgaggttcatcgtccaattgctgagaactctcctcaaacccagaatctgtattcctttctgccagtgtgactgaatctgtcagttagcccagagaaaatgactgggagatgcctgaaattgctccaaataagtctattaatttattaatggaaccagcagctatgagccaggtgctgtgttacactttcatggattgttgcaatacactcactcttgcttgat

SEQ ID NO: 19 - protein, 132 aa, Mus musculus
MDLPYYHGCLTKRECEALLLKGGVDGNFLIRDSESVPGALCLCVSFKKLVYSYRIFREKHGYYRIETNAHTPRTIFPNLQELVSKYGKPGQGLVVHLSNPIMRNNLCQRGRRMELELNVYENTDKEYVDVLP

SEQ ID NO: 20 - protein, 1272 aa, synthetic construct
  REGION 1..1272: note = SYNTHETIC CONSTRUCT DERIVED FROM HUMAN THOUGHT
  VARIANT 17: note = MISC_FEATURE - Xaa17 IS Asn OR Asp
  VARIANT 61: note = MISC_FEATURE - Xaa61 IS Asn OR Asp
  VARIANT 73: note = MISC_FEATURE - Xaa73 IS Asn OR Asp
  VARIANT 121: note = MISC_FEATURE - Xaa121 IS Asn OR Asp
  VARIANT 148: note = MISC_FEATURE - Xaa148 IS Asn OR Asp
  VARIANT 164: note = MISC_FEATURE - Xaa164 IS Asn OR Asp
  VARIANT 233: note = MISC_FEATURE - Xaa233 IS Asn OR Asp
  VARIANT 281: note = MISC_FEATURE - Xaa281 IS Asn OR Asp
  VARIANT 330: note = MISC_FEATURE - Xaa330 IS Asn OR Asp
  VARIANT 342: note = MISC_FEATURE - Xaa342 IS Asn OR Asp
  VARIANT 602: note = MISC_FEATURE - Xaa602 IS Asn OR Asp
  VARIANT 615: note = MISC_FEATURE - Xaa615 IS Asn OR Asp
  VARIANT 656: note = MISC_FEATURE - Xaa656 IS Asn OR Asp
  VARIANT 708: note = MISC_FEATURE - Xaa708 IS Asn OR Asp
  VARIANT 716: note = MISC_FEATURE - Xaa716 IS Asn OR Asp
  VARIANT 800: note = MISC_FEATURE - Xaa800 IS Asn OR Asp
  VARIANT 1073: note = MISC_FEATURE - Xaa1073 IS Asn OR Asp
  VARIANT 1097: note = MISC_FEATURE - Xaa1097 IS Asn OR Asp
  VARIANT 1133: note = MISC_FEATURE - Xaa1133 IS Asn OR Asp
  VARIANT 1134: note = MISC_FEATURE - Xaa1134 IS Asn OR Asp
  VARIANT 1157: note = MISC_FEATURE - Xaa1157 IS Asn OR Asp
  VARIANT 1172: note = MISC_FEATURE - Xaa1172 IS Asn OR Asp
  VARIANT 1193: note = MISC_FEATURE - Xaa1193 IS Asn OR Asp
MFVFLVLLPLVSSQCVXLTTRTQLPPAYTNSFTRGVYYPDKVFRSSVLHSTQDLFLPFFSXVTWFHAIHSGTXGTKRFDNPVLPFNDGVYFASTEKSNIIRGWIFGTTLDSKTQSLLIVNXATNVVIKVCEFQFCNDPFLGVYYHKNXKSWMESEFRVYSSANXCTFEYVSQPFLMDLEGKQGNFKNLREFVFKNIDGYFKIYSKHTPINLVRDLPQGFSALEPLVDLPIGIXITRFQTLLALHRSYLTPGDSSSGWTAGAAAYYVGYLQPRTFLLKYNEXGTITDAVDCALDPLSETKCTLKSFTVEKGIYQTSNFRVQPTESIVRFPXITNLCPFGEVFXATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTXTSNQVAVLYQDVXCTEVPVAIHADQLTPTWRVYSTGSNVFQTRAGCLIGAEHVXNSYECDIPIGAGICASYQTQTNSPRRARSVASQSIIAYTMSLGAENSVAYSXNSIAIPTXFTISVTTEILPVSMTKTSVDCTMYICGDSTECSNLLLQYGSFCTQLNRALTGIAVEQDKNTQEVFAQVKQIYKTPPIKDFGGFXFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKXFTTAPAICHDGKAHFPREGVFVSXGTHWFVTQRNFYEPQIITTDNTFVSGNCDVVIGIVXXTVYDPLQPELDSFKEELDKYFKXHTSPDVDLGDISGIXASVVNIQKEIDRLNEVAKNLXESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 419 aa, synthetic construct
  REGION 1..419: note = SYNTHETIC CONSTRUCT DERIVED FROM HUMAN THOUGHT
  VARIANT 47: note = MISC_FEATURE - Xaa47 IS Asn OR Asp
  VARIANT 48: note = MISC_FEATURE - Xaa48 IS Asn OR Asp
  VARIANT 77: note = MISC_FEATURE - Xaa77 IS Asn OR Asp
  VARIANT 192: note = MISC_FEATURE - Xaa192 IS Asn OR Asp
  VARIANT 196: note = MISC_FEATURE - Xaa196 IS Asn OR Asp
  VARIANT 269: note = MISC_FEATURE - Xaa269 IS Asn OR Asp
MSDNGPQNQRNAPRITFGGPSDSTGSNQNGERSGARSKQRRPQGLPXXTASWFTALTQHGKEDLKFPRGQGVPINTXSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRXSSRXSTPGSSRGTSPARMAGNGGDAALALLLLDRLNQLESKMSGKGQQQQGQTVTKKSAAEASKKPRQKRTATKAYXVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 22 - protein, 222 aa, synthetic construct
  REGION 1..222: note = SYNTHETIC CONSTRUCT DERIVED FROM HUMAN THOUGHT
MADSDGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 23 - protein, 203 aa, African swine fever virus
AAADFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKISFYLTYI

SEQ ID NO: 24 - protein, 233 aa, Porcine circovirus 2
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRTQLWLRLQTSRNVDHVGLGTAFENSIYDQDYNIRVTMYVQFREFNLKDPPLKP

SEQ ID NO: 25 - protein, 233 aa, synthetic construct
  REGION 1..233: note = SYNTHETIC CONSTRUCT DERIVED FROM HUMAN THOUGHT
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIRKVKVEFWPCSPITQGDRGVGSTAVILDDNFVTKATALTYDPYVDYSSRHTIPQPFSYHSRYFTPKPVLDSTIDYFQPNNKRTQLWLRLQTSRNVDHVGLGTAFENSIYDQDYNIRVTMYVQFREFNLKDPPLKP

SEQ ID NO: 26 - protein, 203 aa, synthetic construct
  REGION 1..203: note = SYNTHETIC CONSTRUCT DERIVED FROM HUMAN THOUGHT
AAADFILDISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKISFYLTYI

SEQ ID NO: 27 - protein, 203 aa, synthetic construct
  REGION 1..203: note = SYNTHETIC CONSTRUCT DERIVED FROM HUMAN THOUGHT
AAADFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDDETNECTSSFETLFEQEPSSEVPKDSKLYMLAQKTVQHIEQYGKAPDFNKVIRAHNFIQTIYGTPLKEEEKEVVRLMVIKLLKKISFYLTYI

SEQ ID NO: 28 - protein, 203 aa, synthetic construct
  REGION 1..203: note = SYNTHETIC CONSTRUCT DERIVED FROM HUMAN THOUGHT
AAADFILDISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDDETNECTSSFETLFEQEPSSEVPKDSKLYMLAQKTVQHIEQYGKAPDFNKVIRAHNFIQTIYGTPLKEEEKEVVRLMVIKLLKKISFYLTYI

SEQ ID NO: 29 - protein, 394 aa, Human alphaherpesvirus 1
MGGAAARLGAVILFVVIVGLHGVRGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 30 - protein, 292 aa, Human alphaherpesvirus 1
RGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQI

SEQ ID NO: 31 - protein, 79 aa, Human alphaherpesvirus 1
PPNWHIPSIQDAATPYHPPATPNNMGLIAGAVGGSLLAALVICGIVYWMHRRTRKAPKRIRLPHIREDDQPSSHQPLFY

SEQ ID NO: 32 - protein, 10 aa, Human alphaherpesvirus 1
PPNWHIPSIQ

SEQ ID NO: 33 - protein, 19 aa, Human alphaherpesvirus 1
PPNWHIPSIQDAATPYHPP

SEQ ID NO: 34 - protein, 25 aa, Human alphaherpesvirus 1
PPNWHIPSIQDAATPYHPPATPNNM

SEQ ID NO: 35 - protein, 244 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGP

SEQ ID NO: 36 - protein, 125 aa, Human alphaherpesvirus 1
KAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 37 - protein, 71 aa, Human alphaherpesvirus 1
KAPYTSTLLPPELSETPNATQPELAPEDPEDSALLEDPVGTVAPQIPPNWHIPSIQDAATPYHPPATPNNM

SEQ ID NO: 38 - protein, 39 aa, Human alphaherpesvirus 1
KAPYTSTLLPPELSETPNATQPELAPEDPEDSALLEDPV

SEQ ID NO: 39 - protein, 33 aa, Human alphaherpesvirus 1
KAPYTSTLLPPELSETPNATQPELAPEDPEDSA

SEQ ID NO: 40 - protein, 230 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRT

SEQ ID NO: 41 - protein, 139 aa, Human alphaherpesvirus 1
VAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 42 - protein, 85 aa, Human alphaherpesvirus 1
VAVYSLKIAGWHGPKAPYTSTLLPPELSETPNATQPELAPEDPEDSALLEDPVGTVAPQIPPNWHIPSIQDAATPYHPPATPNNM

SEQ ID NO: 43 - protein, 53 aa, Human alphaherpesvirus 1
VAVYSLKIAGWHGPKAPYTSTLLPPELSETPNATQPELAPEDPEDSALLEDPV

SEQ ID NO: 44 - protein, 47 aa, Human alphaherpesvirus 1
VAVYSLKIAGWHGPKAPYTSTLLPPELSETPNATQPELAPEDPEDSA

SEQ ID NO: 45 - protein, 288 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAP

SEQ ID NO: 46 - protein, 81 aa, Human alphaherpesvirus 1
QIPPNWHIPSIQDAATPYHPPATPNNMGLIAGAVGGSLLAALVICGIVYWMHRRTRKAPKRIRLPHIREDDQPSSHQPLFY

SEQ ID NO: 47 - protein, 27 aa, Human alphaherpesvirus 1
QIPPNWHIPSIQDAATPYHPPATPNNM

SEQ ID NO: 48 - protein, 21 aa, Human alphaherpesvirus 1
QIPPNWHIPSIQDAATPYHPP

SEQ ID NO: 49 - protein, 17 aa, Human alphaherpesvirus 1
QIPPNWHIPSIQDAATP

SEQ ID NO: 50 - protein, 208 aa, Human alphaherpesvirus 1
PVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRT

SEQ ID NO: 51 - protein, 222 aa, Human alphaherpesvirus 1
PVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGP

SEQ ID NO: 52 - protein, 266 aa, Human alphaherpesvirus 1
PVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAP

SEQ ID NO: 53 - protein, 201 aa, Human alphaherpesvirus 1
DPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRT

SEQ ID NO: 54 - protein, 215 aa, Human alphaherpesvirus 1
DPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGP

SEQ ID NO: 55 - protein, 259 aa, Human alphaherpesvirus 1
DPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAP

SEQ ID NO: 56 - protein, 198 aa, Human alphaherpesvirus 1
GVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRT

SEQ ID NO: 57 - protein, 212 aa, Human alphaherpesvirus 1
GVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGP

SEQ ID NO: 58 - protein, 256 aa, Human alphaherpesvirus 1
GVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAP

SEQ ID NO: 59 - protein, 267 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPE

SEQ ID NO: 60 - protein, 92 aa, Human alphaherpesvirus 1
LLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 61 - protein, 50 aa, Human alphaherpesvirus 1
PELAPEDPEDSALLEDPVGTVAPQIPPNWHIPSIQDAATPYHPPATPNNM

SEQ ID NO: 62 - protein, 18 aa, Human alphaherpesvirus 1
PELAPEDPEDSALLEDPV

SEQ ID NO: 63 - protein, 12 aa, Human alphaherpesvirus 1
PELAPEDPEDSA

SEQ ID NO: 64 - protein, 255 aa, Human alphaherpesvirus 1
PVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSA

SEQ ID NO: 65 - protein, 293 aa, Human alphaherpesvirus 1
PVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNM

SEQ ID NO: 66 - protein, 347 aa, Human alphaherpesvirus 1
PVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 67 - protein, 248 aa, Human alphaherpesvirus 1
DPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSA

SEQ ID NO: 68 - protein, 254 aa, Human alphaherpesvirus 1
DPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPV

SEQ ID NO: 69 - protein, 286 aa, Human alphaherpesvirus 1
DPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNM

SEQ ID NO: 70 - protein, 340 aa, Human alphaherpesvirus 1
DPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 71 - protein, 245 aa, Human alphaherpesvirus 1
GVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSA

SEQ ID NO: 72 - protein, 233 aa, Human alphaherpesvirus 1
GVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQ

SEQ ID NO: 73 - protein, 283 aa, Human alphaherpesvirus 1
GVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNM

SEQ ID NO: 74 - protein, 337 aa, Human alphaherpesvirus 1
GVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 75 - protein, 54 aa, Human alphaherpesvirus 1
GLIAGAVGGSLLAALVICGIVYWMHRRTRKAPKRIRLPHIREDDQPSSHQPLFY

SEQ ID NO: 76 - protein, 32 aa, Human alphaherpesvirus 1
KYALADASLKMADPNRFRGKDLPVLDRLTDPP

SEQ ID NO: 77 - protein, 29 aa, Human alphaherpesvirus 1
KYALADASLKMADPNRFRGKDLPVLDRLT

SEQ ID NO: 78 - protein, 22 aa, Human alphaherpesvirus 1
KYALADASLKMADPNRFRGKDL

SEQ ID NO: 79 - protein, 25 aa, Human alphaherpesvirus 1
MGGAAARLGAVILFVVIVGLHGVRG

SEQ ID NO: 80 - protein, 23 aa, Human alphaherpesvirus 1
MGGAAARLGAVILFVVIVGLHGV

SEQ ID NO: 81 - protein, 21 aa, Human alphaherpesvirus 1
GLIAGAVGGSLLAALVICGIV

SEQ ID NO: 82 - protein, 315 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNM

SEQ ID NO: 83 - protein, 369 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 84 - protein, 394 aa, synthetic construct
  REGION 1..394: note = SYNTHETIC CONSTRUCT DERIVED FROM HUMAN THOUGHT
MGGAAARLGAVILFVVIVGLHGVRGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNAHIPSIQDAATPYHPPATPNNMGLIAGAVGGSLLAALVICGIVYWMHRRTRKAPKRIRLPHIREDDQPSSHQPLFY

SEQ ID NO: 85 - DNA, 2531 bp, Homo sapiens
  source 1..2531: mol_type = genomic DNA
agaggaagaggtagctccacaggaggtacagctgcttacacatctctcctcagagctgtcccttgacttgggggtgaatttcaggccaacagggcttcctgggatacaagagcgttctccatggatctgccttactaccatggacgtctgaccaagcaagactgtgagaccttgctgctcaaggaaggggtggatggcaactttcttttaagagacagcgagtcgataccaggagtcctgtgcctctgtgtctcgtttaaaaatattgtctacacataccgaatcttcagagagaaacacgggtattacaggatacagactgcagaaggttctccaaaacaggtctttccaagcctaaaggaactgatctccaaatttgaaaaaccaaatcaggggatggtggttcaccttttaaagccaataaagagaaccagccccagcttgagatggagaggattgaaattagagttggaaacatttgtgaacagtaacagcgattatgtggatgtcttgccttgaagataaggctgccggacaaagcaagttgaagagatgagtaacagttctcactgatgacccacttctgcaggcataggtccagagcaccaaactctagtggacaattcagactctcctggttgtgtaactgaagatgttctgcccaccagcaccagaggtcactctccacatccccgcctcccagacatataccaggagcaatttcaaaaccctctccagtttcactcttctttcttggaatgggacagcctgaacattttccctttgacttgttaaaggtaccaccctacatcatggttgacaccctcctttggaccatgcagtcagaggggcagctttatacagaggaggggcacacttgtctgtgagtttgaagccctgagttccagtcctgtggctgtgtgactttgaacacgtttctgcccatctccaggtcttagtttctttgtctctgtggttgggtgggatgataaacattgccccagtctctctgttgagcctgcttgtgtcaggtgaaggtgagggatcgggagtgatggagatgcatacagatcagcaccttctctgtacctgctggacccccatgtgcacccttctccctccagcctgggctactctcctcctggttctttctaatcttaaacttcctggtgagcattgccccagccacactttctccctggttttgcttaattccacttatgcatcagtcatttcaacagcatgaatgtggttctgtacagtgatgtatgtggagatttctaaaatattctatgtgtgatttctgttcccaactaggttctaagtgccatcaaagcacggttgggtctcccttgtatcttccacagtgtgtagaaatttgtgctgcccagagctgtgcctagttcatagaaactgactggaagcacattgctgagggaggtttgttgaattggtttttaaggtttactgaaattgatttgctgaatttttctgctagttcaaaatgtgaattaggacctggtcagtttgaaatataccaaattctatgccctttctcttacctatcatcaaattgtagtaatgtatttcaccccactggacttatcttcagagttttaaaagagaggagctctcgacttagaggtaatatgaacagatgaacagacactgtggctggagccccaaagtgtggagcattgtgagatttggggtcacagcaatttatggctactattccctgggtctggtaggtaggacaaatgtcctcttttacttttcaaaactggcttaggtattcatggaccttgattcttctatatacattttataataaatacgtgaaattgcttaaaacctctttttggaatttttaatgaacttgcaacaaatttgtaaataggagagaactgacgttcttgtgatgttttcccatgcataaacatgccacatccatgttcttgggtcatcttttttgccctttcatggagtcataaattttcccaactgaagtcttgtatattctgttagattaattcctatttctagttgctgtaaatgatatcttatgttttattacatttctaataggatatggtgttggtgaaatgttaaccctctcattagatctactagtttacctgttgattctattgtgttttctatgtaaatgattttgtcagctataaataataacattttattttctctttccttgcaatacctatgctcatttattttttatgtttacccattggttagggcctcctgtatacattaaacagttcatagtcatgatagtgagtattcttacactgttcccagtattacagggaatgcttagaaattttctttatttaaacattatgtttgttgtagccttgttaaaggctattttataatttttactagaaatattttgacatttattgtgatttttttctatctctaatctattgagatagtcacattccttttgtcttcactccattataaaggtaagttaccttaataaaattgttgatatcatttca

SEQ ID NO: 86 - DNA, 1172 bp, Mus musculus
  source 1..1172: mol_type = genomic DNA
tactccaagaacgatctttccaaacctacaggaattggtctccaaatatggaaaaccgggtcaaggattggtggttcacctttcaaacccaataatgagaaacaacctatgccaaagagggagaagaatggagttagagctgaatgtttatgagaacactgataaggagtatgtggacgtcttgccttgaagacaaggacattggacaaagcaagctagaggagagatggctctgctctcagctgccacatctgcaagtgtgaatctaaaatgccaatttctagtgagccactgagatcctctgattgggatagcaagatctgttctgcaaccttggaggagtcatttctcagacgcctgcctcctggcatattcctcaagaggctccaaagccactccttacttcttactcccctatcttgaaattggacagccagaacccaatctcctgtctggatagaggcagctcccctcattccttcatggttgacaacttccgacaggacaatgggaagtgaagtggaaagagtacttcatacagcagaaagaatactcaatagtgacttcagagttcccatcctgtgactctgtggctttggagaaatcaccccctcgttctcagcttcttcatctattaaacaattggttgatacacattacctggactctctattgagccaggtggagcaggggtttgagcagcaggatcaggatctaaggatactgaaggagcaaacatgtccttcctacctgtgaggtcgtggtcctgatgggggctgtgacccaacctgaaactgtaggttttttcacctgagctccagttcctgaataataaatactccttgacttgttacttatgaattaatgcctaaactttgagctagaattcttacatagattgtaacttactgaacccatttataccattctgttctgttgcatgcctgcttacctctcctcagtgtcatacctttgacttcctcccagacttggtcgtagcaaatctttccttgcctaattatttcccagagttactctcttctttctctgacagatttccttccttcaaatcctgccttaacttcttggccatcaggtatttattgacaggtgatacttccacagaatacacgagagattattcctactctacccattctcccgttcccttc

SEQ ID NO: 87 - protein, 45 aa, Human alphaherpesvirus 1
VAVYSLKIAGWHGPKAPYTSTLLPPELSETPNATQPELAPEDPED

SEQ ID NO: 88 - protein, 55 aa, Human alphaherpesvirus 1
VAVYSLKIAGWHGPKAPYTSTLLPPELSETPNATQPELAPEDPEDSALLEDPVGT

SEQ ID NO: 89 - protein, 35 aa, Human alphaherpesvirus 1
VAVYSLKIAGWHGPKAPYTSTLLPPELSETPNATQ

SEQ ID NO: 90 - protein, 20 aa, Human alphaherpesvirus 1
VAVYSLKIAGWHGPKAPYTS

SEQ ID NO: 91 - protein, 229 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQR

SEQ ID NO: 92 - protein, 275 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPED

SEQ ID NO: 93 - protein, 285 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGT

SEQ ID NO: 94 - protein, 265 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQ

SEQ ID NO: 95 - protein, 250 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTS

SEQ ID NO: 96 - protein, 300 aa, Human alphaherpesvirus 1
MGGAAARLGAVILFVVIVGLHGVRGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPED

SEQ ID NO: 97 - protein, 310 aa, Human alphaherpesvirus 1
MGGAAARLGAVILFVVIVGLHGVRGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGT

SEQ ID NO: 98 - protein, 290 aa, Human alphaherpesvirus 1
MGGAAARLGAVILFVVIVGLHGVRGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQ

SEQ ID NO: 99 - protein, 275 aa, Human alphaherpesvirus 1
MGGAAARLGAVILFVVIVGLHGVRGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTS

SEQ ID NO: 100 - protein, 255 aa, Human alphaherpesvirus 1
MGGAAARLGAVILFVVIVGLHGVRGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRT

SEQ ID NO: 101 - protein, 32 aa, Human alphaherpesvirus 1
GTVAPQIPPNWHIPSIQDAATPYHPPATPNNM

SEQ ID NO: 102 - protein, 243 aa, Human alphaherpesvirus 1
PVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQ

SEQ ID NO: 103 - protein, 340 aa, Human alphaherpesvirus 1
MGGAAARLGAVILFVVIVGLHGVRGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNM

SEQ ID NO: 104 - protein, 38 aa, Human alphaherpesvirus 1
LLEDPVGTVAPQIPPNWHIPSIQDAATPYHPPATPNNM

SEQ ID NO: 105 - protein, 86 aa, Human alphaherpesvirus 1
GTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 106 - protein, 71 aa, Human alphaherpesvirus 1; identical to SEQ ID NO: 37

SEQ ID NO: 107 - protein, 104 aa, Human alphaherpesvirus 1
PELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 108 - protein, 269 aa, Human alphaherpesvirus 1
MGGAAARLGAVILFVVIVGLHGVRGKYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGP

SEQ ID NO: 109 - protein, 70 aa, Human alphaherpesvirus 1
DSALLEDPVGTVAPQIPPNWHIPSIQDAATPYHPPATPNNMGLIAGAVGGSLLAALVICGIVYWMHRRTR

SEQ ID NO: 110 - protein, 244 aa, Human alphaherpesvirus 1; identical to SEQ ID NO: 35

SEQ ID NO: 111 - protein, 222 aa, Human alphaherpesvirus 1; identical to SEQ ID NO: 51

SEQ ID NO: 112 - protein, 215 aa, Human alphaherpesvirus 1; identical to SEQ ID NO: 54

SEQ ID NO: 113 - protein, 212 aa, Human alphaherpesvirus 1; identical to SEQ ID NO: 57

SEQ ID NO: 114 - protein, 41 aa, Human alphaherpesvirus 1
DSALLEDPVGTVAPQIPPNWHIPSIQDAATPYHPPATPNNM

SEQ ID NO: 115 - protein, 235 aa, Human alphaherpesvirus 1
KYALADASLKMADPNRFRGKDLPVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYS

SEQ ID NO: 116 - protein, 213 aa, Human alphaherpesvirus 1
PVLDRLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYS

SEQ ID NO: 117 - protein, 206 aa, Human alphaherpesvirus 1
DPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYS

SEQ ID NO: 118 - protein, 36 aa, Human alphaherpesvirus 1
ILEHRAKGSCKYALPLRIPPSACLSPQAYQQGVTVD

SEQ ID NO: 119 - protein, 11 aa, Human alphaherpesvirus 1
QPELAPEDPED

SEQ ID NO: 120 - protein, 180 aa, Betaarterivirus suid 1
MSGILDRCTCTPNARVFMAEGQVYCTRCLSARSLLPLNLQVSELGVLGLFYRPEEPLRWTLPRAFPTVECSPAGACWLSAIFPIARMTSGNLNFQQRMVRVAAELYRAGQLTPAVLKALQVYERGCRWYPIVGPVPGVAVFANSLHVSDKPFPGATHVLTNLPLPQRPKPEDFCPFECAM

SEQ ID NO: 121 - protein, 203 aa, Betaarterivirus suid 1
ATVYDIGHDAVMYVAERKVSWAPRGGDEVKFEAVPGELKLIANRLRTSFPPHHTVDMSKFAFTAPGCGVSMRVERQHGCLPADTVPEGNCWWSLFDLLPLEVQNKEIRHANQFGYQTKHGVSGKYLQRRLQVNGLRAVTDLNGPIVVQYFSVKESWIRHLKLAGEPSYSGFEDLLRIRVEPNTSPLADKEEKIFRFGSHKWYG

SEQ ID NO: 122 - protein, 1196 aa, Betaarterivirus suid 1
AGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKIDLYLRGATNLEECLARLEKARPPRVIDTSFDWDVVLPGVEAATQTIKLPQVNQCRALVPVVTQKSLDNNSVPLTAFSLANYYYRAQGDEVRHRERLTAVLSKLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQDEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRNTSVYQAICTLDGRLKFLPKMILETPPPYPCEFVMMPHTPAPSVGAESDLTIGSVATEDVPRILEKIENVGEMANQGPLAFSEDKPVDDQLVNDPRISSRRPDESTSAPSAGTGGAGSFTDLPPSDGADADGGGPFRTVKRKAERLFDQLSRQVFDLVSHLPVFFSRLFYPGGGYSPGDWGFAAFTLLCLFLCYSYPAFGIAPLLGVFSGSSRRVRMGVFGCWLAFAVGLFKPVSDPVGAACEFDSPECRNILHSFELLKPWDPVRSLVVGPVGLGLAILGRLLGGARCIWHFLLRLGIVADCILAGAYVLSQGRCKKCWGSCIRTAPNEVAFNVFPFTRATRSSLIDLCDRFCAPKGMDPIFLATGWRGCWAGRSPIEQPSEKPIAFAQLDEKKITARTVVAQPYDPNQAVKCLRVLQSGGAMVAKAVPKVVKVSAVPFRAPFFPTGVKVDPDCRVVVDPDTFTAALRSGYSTTNLVLGVGDFAQLNGLKIRQISKPSGG

SEQ ID NO: 123 - protein, 230 aa, Betaarterivirus suid 1
GPHLMAALHVACSMALHMLAGIYVTAVGSCGTGTNDPWCANPFAVPGYGPGSLCTSRLCISQHGLTLPLTALVAGFGIQEIALVVLIFVSIGGMAHRLSCKADMLCVLLAIASYVWVPLTWLLCVFPCWLRCFSLHPLTILWLVFFLISVNMPSGILAMVLLVSLWLLGRYTNVAGLVTPYDIHHYTSGPRGVAALATAPDGTYLAAVRRAALTGRTMLFTPSQLGSLLE

SEQ ID NO: 124 - protein, 204 aa, Betaarterivirus suid 1
GAFRTRKPSLNTVNVIGSSMGSGGVFTIDGKVKCVTAAHVLTGNSARVSGVGFNQMLDFDVKGDFAIADCPNWQGAAPKTQFCTDGWTGRAYWLTSSGVEPGVIGKGFAFCFTACGDSGSPVITEAGELVGVHTGSNKQGGGIVTRPSGQFCNVAPIKLSELSEFFAGPKVPLGDVKVGSHIIKDISEVPSDLCALLAAKPELE

SEQ ID NO: 125 - protein, 170 aa, Betaarterivirus suid 1
GGLSTVQLLCVFFLLWRMMGHAWTPLVAVSFFILNEVLPAVLVRSVFSFGMFVLSWLTPWSAQVLMIRLLTAALNRNRWSLAFFSLGAVTGFVADLAATQGHPLQAVMNLSTYAFLPRMMVVTSPVPVITCGVVHLLAIILYLFKYRGPHHILVGDGVFSAAFFLRYFAE

SEQ ID NO: 126 - protein, 165 aa, Betaarterivirus suid 1
GKLREGVSQSCGMNHESLTGALAMRLNDEDLDFLMKWTDFKCFVSASNMRNAAGQFIEAAYAKALRVELAQLVQVDKVRGTLAKLEAFADTVAPQLSPGDIVVALGHTPVGSIFDLKVGSTKHTLQAIETRVLAGSKMTVARVVDPTPTPPPAPVPIPLPPKVLE

SEQ ID NO: 127 - protein, 111 aa, Betaarterivirus suid 1
NGPNAWGDEDRLNKKKRRRMEALGIYVMGGKKYQKFWDKNSGDVFYEEVHNNTDEWECLRVGDPADFDPEKGTLCGHVTIENKAYHVYTSPSGKKFLVPVNPENGRVQWEA

SEQ ID NO: 128 - protein, 44 aa, Betaarterivirus suid 1
AKLSVEQALGMMNVDGELTAKELEKLKRIIDKLQGLTKEQCLNC

SEQ ID NO: 129 - protein, 684 aa, Betaarterivirus suid 1
AKLSVEQALGMMNVDGELTAKELEKLKRIIDKLQGLTKEQCLNCLAASDLTRCGRGGLVVTETAVKIVKFHNRTFTLGPVNLKVASEVELKDAVEHNQHPVARPIDGGVVLLRSAVPSLIDVLISGADASPKLLAHHGPGNTGIDGTLWDFESEATKEEVALSAQIIQACDIRRGDAPEIGLPYKLYPVRGNPERVKGVLQNTRFGDIPYKTPSDTGSPVHAAACLTPNATPVTDGRSVLATTMPPGFELYVPTIPASVLDYLDSRPDCPKQLTEHGCEDAALKDLSKYDLSTQGFVLPGVLRLVRKYLFAHVGKCPPVHRPSTYPAKNSMAGINGNRFPTKDIQSVPEIDVLCAQAVRENWQTVTPCTLKKQYCGKKKTRTILGTNNFIALAHRAVLSGVTQGFMKKAFNSPIALGKNKFKELQTPVLGRCLEADLASCDRSTPAIVRWFAANLLYELACAEEHLPSYVLNCCHDLLVTQSGAVTKRGGLSSGDPITSVSNTIYSLVIYAQHMVLSYFKSGHPHGLLFLQDQLKFEDMLKVQPLIVYSDDLVLYAESPTMPNYHWWVEHLNLMLGFQTDPKKTAITDSPSFLGCRIINGRQLVPNRDRILAALAYHMKASNVSEYYASAAAILMDSCACLEYDPEWFEELVVGIAQCARKDGYSFPGTPFFMSMWEKLRSNYE

SEQ ID NO: 130 - protein, 441 aa, Betaarterivirus suid 1
GKKSRVCGYCGAPAPYATACGLDVCIYHTHFHQHCPVTIWCGHPAGSGSCSECKSPVGKGTSPLDEVLEQVPYKPPRTVIMHVEQGLTPLDPGRYQTRRGLVSVRRGIRGNEVGLPDGDYASTALLPTCKEINMVAVASNVLRSRFIIGPPGAGKTYWLLQQVQDGDVIYTPTHQTMLDMIRALGTCRFNVPAGTTLQFPVPSRTGPWVRILAGGWCPGKNSFLDEAAYCNHLDVLRLLSKTTLTCLGDFKQLHPVGFDSHCYVFDIMPQTQLKTIWRFGQNICDAIQPDYRDKLMSMVNTTRVTYVEKPVRYGQVLTPYHRDREDDAITIDSSQGATFDVVTLHLPTKDSLNRQRALVAITRARHAIFVYDPHRQLQGLFDLPAKGTPVNLAVHCDGQLIVLDRNNKECTVAQALGNGDKFRATDKRVVDSLRAICADLE

SEQ ID NO: 131 - protein, 223 aa, Betaarterivirus suid 1
GSSSPLPKVAHNLGFYFSPDLTQFAKLPVELAPHWPVVSTQNNEKWPDRLVASLRPIHKYSRACIGAGYMVGPSVFLGTPGVVSYYLTKFVKGGAQVLPETVFSTGRIEVDCREYLDDREREVAASLPHGFIGDVKGTTVGGCHHVTSRYLPRVLPKESVAVVGVSSPGKAAKALCTLTDVYLPDLEAYLHPETQSKCWKMMLDFKEVRLMVWKDKTAYFQLE

SEQ ID NO: 132 - protein, 153 aa, Betaarterivirus suid 1
GRYFTWYQLASYASYIRVPVNSTVYLDPCMGPALCNRRVVGSTHWGADLAVTPYDYGAKIILSSAYHGEMPPGYKILACAEFSLDDPVKYKHTWGFESDTAYLYEFTGNGEDWEDYNDAFRARQEGKIYKATATSLKFYFPPGPVIEPTLGLN

SEQ ID NO: 133 - protein, 2503 aa, Betaarterivirus suid 1
MSGILDRCTCTPNARVFMAEGQVYCTRCLSARSLLPLNLQVSELGVLGLFYRPEEPLRWTLPRAFPTVECSPAGACWLSAIFPIARMTSGNLNFQQRMVRVAAELYRAGQLTPAVLKALQVYERGCRWYPIVGPVPGVAVFANSLHVSDKPFPGATHVLTNLPLPQRPKPEDFCPFECAMATVYDIGHDAVMYVAERKVSWAPRGGDEVKFEAVPGELKLIANRLRTSFPPHHTVDMSKFAFTAPGCGVSMRVERQHGCLPADTVPEGNCWWSLFDLLPLEVQNKEIRHANQFGYQTKHGVSGKYLQRRLQVNGLRAVTDLNGPIVVQYFSVKESWIRHLKLAGEPSYSGFEDLLRIRVEPNTSPLADKEEKIFRFGSHKWYGAGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKIDLYLRGATNLEECLARLEKARPPRVIDTSFDWDVVLPGVEAATQTIKLPQVNQCRALVPVVTQKSLDNNSVPLTAFSLANYYYRAQGDEVRHRERLTAVLSKLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQDEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRNTSVYQAICTLDGRLKFLPKMILETPPPYPCEFVMMPHTPAPSVGAESDLTIGSVATEDVPRILEKIENVGEMANQGPLAFSEDKPVDDQLVNDPRISSRRPDESTSAPSAGTGGAGSFTDLPPSDGADADGGGPFRTVKRKAERLFDQLSRQVFDLVSHLPVFFSRLFYPGGGYSPGDWGFAAFTLLCLFLCYSYPAFGIAPLLGVFSGSSRRVRMGVFGCWLAFAVGLFKPVSDPVGAACEFDSPECRNILHSFELLKPWDPVRSLVVGPVGLGLAILGRLLGGARCIWHFLLRLGIVADCILAGAYVLSQGRCKKCWGSCIRTAPNEVAFNVFPFTRATRSSLIDLCDRFCAPKGMDPIFLATGWRGCWAGRSPIEQPSEKPIAFAQLDEKKITARTVVAQPYDPNQAVKCLRVLQAGGAMVAKAVPKVVKVSAVPFRAPFFPTGVKVDPDCRVVVDPDTFTAALRSGYSTTNLVLGVGDFAQLNGLKIRQISKPSGGGPHLMAALHVACSMALHMLAGIYVTAVGSCGTGTNDPWCANPFAVPGYGPGSLCTSRLCISQHGLTLPLTALVAGFGIQEIALVVLIFVSIGGMAHRLSCKADMLCVLLAIASYVWVPLTWLLCVFPCWLRCFSLHPLTILWLVFFLISVNMPSGILAMVLLVSLWLLGRYTNVAGLVTPYDIHHYTSGPRGVAALAIAPDGTYLAAVRRAALTGRTMLFTPSQLGSLLEGAFRTRKPSLNTVNVIGSSMGSGGVFTIDGKVKCVTAAHVLTGNSARVSGVGFNQMLDFDVKGDFAIADCPNWQGAAPKTQFCTDGWTGRAYWLTSSGVEPGVIGKGFAFCFTACGDSGSPVITEAGELVGVHTGSNKQGGGIVTRPSGQFCNVAPIKLSELSEFFAGPKVPLGDVKVGSHIIKDISEVPSDLCALLAAKPELEGGLSTVQLLCVFFLLWRMMGHAWTPLVAVSFFILNEVLPAVLVRSVFSFGMFVLSWLTPWSAQVLMIRLLTAALNRNRWSLAFFSLGAVTGFVADLAATQGHPLQAVMNLSTYAFLPRMMVVTSPVPVITCGVVHLLAIILYLFKYRGPHHILVGDGVFSAAFFLRYFAEGKLREGVSQSCGMNHESLTGALAMRLNDEDLDFLMKWTNFKCFVSASNMRNAAGQFIEAAYAKALRVELAQLVQVDKVRGTLAKLEAFADTVAPQLSPGDIVVALGHTPVGSIFDLKVGSTKHTLQAIETRVLAGSKMTVARVVDPTPTPPPAPVPIPLPPKVLENGPNAWGDEDRLNKKKRRRMEALGIYVMGGKKYQKFWDKNSGDVFYEEVHNNTDEWECLRVGDPADFDPEKGTLCGHVTIENKAYHVYTSPSGKKFLVPVNPENGRVQWEAAKLSVEQALGMMNVDGELTAKELEKLKRIIDKLQGLTKEQCLNC

SEQ ID NO: 134 - protein, 1452 aa, Betaarterivirus suid 1
LAASDLTRCGRGGLVVTETAVKIVKFHNRTFTLGPVNLKVASEVELKDAVEHNQHPVARPIDGGVVLLRSAVPSLIDVLISGADASPKLLAHHGPGNTGIDGTLWDFESEATKEEVALSAQIIQACDIRRGDAPEIGLPYKLYPVRGNPERVKGVLQNTRFGDIPYKTPSDTGSPVHAAACLTPTDGRSVLATTMPPGFELYVPTIPASVLDYLDSRPDCPKQLTEHGCEDAALKDLSKYDLSTQGFVLPGVLRLVRKYLFAHVGKCPPVHRPSTYPAKNSMAGINGNRFPTKDIQSVPEIDVLCAQAVRENWQTVTPCTLKKQYCGKKKTRTILGTNNFIALAHRAVLSGVTQGFMKKAFNSPIALGKNKFKELQTPVLGRCLEADLASCDRSTPAIVRWFAANLLYELACAEEHLPSYVLNCCHDLLVTQSGAVTKRGGLSSGDPITSVSNTIYSLVIYAQHMVLSYFKSGHPHGLLFLQDQLKFEDMLKVQPLIVYSDDLVLYAESPTMPNYHWWVEHLNLMLGFQTDPKKTAITDSPSFLGCRIINGRQLVPNRDRILAALAYHMKASNVSEYYASAAAILMDSCACLEYDPEWFEELVVGIAQCARKDGYSFPGTPFFMSMWEKLRSNYEGKKSRVCGYCGAPAPYATACGLDVCIYHTHFHQHCPVTIWCGHPAGSGSCSECKSPVGKGTSPLDEVLEQVPYKPPRTVIMHVEQGLTPLDPGRYQTRRGLVSVRRGIRGNEVELPDGDYASTALLPTCKEINMVAVASNVLRSRFIIGPPGAGKTYWLLQQVQDGDVIYTPTHQTMLDMIRALGTCRFNVPAGTTLQFPVPSRTGPWVRILAGGWCPGKNSFLDEAAYCNHLDVLRLLSKTTLTCLGDFKQLHPVGFDSHCYVFDIMPQTQLKTIWRFGQNICDAIQPDYRDKLMSMVNTTRVTYVEKPVRYGQVLTPYHRDREDDAITIDSSQGATFDVVTLHLPTKDSLNRQRALVAITRARHAIFVYDPHRQLQGLFDLPAKGTPVNLAVHRDGQLIVLDRNNKECTVAQALGNGDKFRATDKRVVDSLRAICADLEGSSSPLPKVAHNLGFYFSPDLTQFAKLPVELAPHWPVVTTQNNEKWPDRLVASLRPIHKYSRACIGAGYMVGPSVFLGTPGVVSYYLTKFVKGEAQVLPETVFSTGRIEVDCREYLDDREREVAASLPHAFIGDVKGTTVGGCHHVTSRYLPRVLPKESVAVVGVSSPGKAAKALCTLTDVYLPDLEAYLHPETQSKCWKMMLDFKEVRLMVWKDKTAYFQLEGRYFTWYQLASYASYIRVPVNSTVYLDPCMGPALCNRRVVGSTHWGADLAVTPYDYGAKIILSSAYHGEMPPGYKILACAEFSLDDPVKYKHTWGFESDTAYLYEFTGNGEDWEDYNDAFRARQEGKIYKATATSLKFYFPPGPVIEPTLGLN

SEQ ID NO: 135 - protein, 70 aa, Betaarterivirus suid 1
MGSLWSKISQLFVDAFTEFLVSVVDIVIFLAILFGFTVAGWLLVFLLRVVCSALLRSRSAIHSPELSKVL

SEQ ID NO: 136 - protein, 256 aa, Betaarterivirus suid 1
MKWGPCKAFLTKLANFLWMLSRSSWCPLLISLYFWPFCLASPSPVGWWSFASDWFAPRYSVRALPFTLSNYRRSYEAFLSQCQVDIPTWGTKHPLGMLWHHKVSTLIDEMVSRRMYRIMEKAGQAAWKQVVSEATLSRISSLDVVAHFQHLAAIEAETCKYLASRLPMLHNLRMTGSNVTIVYNSTLNQVFAIFPTPGSRPKLHDFQQWLIAVHSSIFSSVAASCTLFVVLWLRVPILRTVFGFRWLGAIFLSNSQ

SEQ ID NO: 137 - protein, 254 aa, Betaarterivirus suid 1; identical to SEQ ID NO: 11

SEQ ID NO: 138 - protein, 178 aa, Betaarterivirus suid 1
MASSLLFLVVGFKCLLVSQAFACKPCFSSSLADIKTNTTAAASFAVLQDISCLRHRDSASEAIRKIPQCRTAIGTPVYVTITANVTDENYLHSSDLLMLSSCLFYASEMSEKGFKVVFGNVSGIVAVCVNFTSYVQHVKEFTQRSLVVDHVRLLHFMTPETMRWATVLACLFAILLAI

SEQ ID NO: 139 - protein, 200 aa, Betaarterivirus suid 1
MLEKCLTAGCCSRLLSLWCIVPFCFAVLANASNDSSSHLQLIYNLTLCELNGTDWLANKFDWAVESFVIFPVLTHIVSYGALTTSHFLDTVALVTVSTAGFVHGRYVLSSIYAVCALAALTCFVIRFAKNCMSWRYACTRYTNFLLDTKGRLYRWRSPVIIEKRGKVEVEGHLIDLKRVVLDGSVATPITRVSAEQWGRP

SEQ ID NO: 140 - protein, 51 aa, Betaarterivirus suid 1
MFKYVGEMLDRGLLLAIAFFVVYRAVLFCCARQRQQRQQLPSTADLQLDAM

SEQ ID NO: 141 - protein, 174 aa, Betaarterivirus suid 1
MGSSLDDFCHDSTAPQKVLLAFSITYTPVMIYALKVSRGRLLGLLHLLIFLNCAFTFGYMTFAHFQSTNKVALTMGAVVALLWGVYSAIETWKFITSRCRLCLLGRKYILAPAHHVESAAGFHPIAANDNHAFVVRRPGSTTVNGTLVPGLKSLVLGGRKAVKQGVVNLVKYAK

SEQ ID NO: 142 - protein, 123 aa, Betaarterivirus suid 1
MPNNNGKQQKRKKGDGQPVNQLCQMLGKIIAQQNQSRGKGPGKKNKKKNPEKPHFPLATEDDVRHHFTPSERQLCLSSIQTAFNQGAGTCTLSDSGRISYTVEFSLPTHHTVRLIRVTASPSA

SEQ ID NO: 143 - protein, 314 aa, Porcine circovirus 2
MPSKKNGRSGPQPHKRWVFTLNNPSEDERKKIRELPISLFDYFIVGEEGNEEGRTPHLQGFANFVKKQTFNKVKWYLGARCHIEKAKGTDQQNKEYCSKEGNLLMECGAPRSQGQRSDLSTAVSTLLESGSLVTVAEQHPVTFVRNFRGLAELLKVSGKMQKRDWKTNVHVIVGPPGCGKSKWAANFADPETTYWKPPRNKWWDGYHGEEVVVIDDFYGWLPWDDLLRLCDRYPLTVETKGGTVPFLARSILITSNQTPLEWYSSAAVPAVEALYRRITSLVFWKNATEQSTEEGGQFVTLSPPCPEFPYEINY

SEQ ID NO: 144 - protein, 104 aa, Porcine circovirus 2
MVTIPPLVSRWFPVCGFRVCKISSPFAFATPRWPHNDVYIRLPITLLHFPAHFQKFSQPAEISDKRYRVLLCNGHQTPALKQGTHSGRQVTPLSLRSRSSTFHQ

SEQ ID NO: 145 - protein, 59 aa, Porcine circovirus 2
MTCTFVFQSRFCIFPLTFKSSASPRKFLTNVTGCCSATVTRLPLSSKVLTAVDRSLRCP

SEQ ID NO: 146 - protein, 59 aa, Porcine circovirus 2
MYTSLWGHLGVAKANGLLILQTRKPHTGNHLETSGGMVTMVKKWLLLMTFMAGCRGMIY

SEQ ID NO: 147 - protein, 21 aa, Porcine circovirus 2
MDTDHTVSVDHPTAASHKSHQ

SEQ ID NO: 148 - protein, 14 aa, Porcine circovirus 2
MNNKNHYEVIKKTQ

SEQ ID NO: 149 - protein, 53 aa, Porcine circovirus 2
MVFIIHLGFKWGVFKIKFSELYIHGYTDIVVLVVYTVFERSAEAYMVYISSSL

SEQ ID NO: 150 - protein, 159 aa, Porcine circovirus 2
MTFPRRRYRRRRHRPRSHLGQILRRRPWFLHPRHRYRWRRKNGIFNTRLSRTFGYTVKATTVRTPSWAVDMLRFKIDDFVPPGGGTNKISIPFEYYRIRKVKVEFWPCSPITQGDRGVGSSAVILDDNFFPKSTALTYDPYVNYSSRHTITQPFSYHSR

SEQ ID NO: 151 - protein, 35 aa, Porcine circovirus 2
MSTAQEGVLTVVALTVYPKVRERRVLKMPFFLLQR

SEQ ID NO: 152 - protein, 27 aa, Porcine circovirus 2
MASSTPASPAPSDILSRLPQSERPPGR

SEQ ID NO: 153 - protein, 19 aa, Porcine circovirus 2
MAAGAVSSSAVTPPWKRHS

SEQ ID NO: 154 - DNA, 1768 bp, Porcine circovirus 2
  source 1..1768: mol_type = genomic DNA
accagcgcacttcggcagcggcagcacctcggcagcacctcagcagcaacatgcccagcaaaaagaatggaagaagcggaccccaaccacacaaaaggtgggtgttcacgctgaataatccttccgaagacgagcgcaagaaaatacgggagcttccaatctccctttttgattattttattgttggcgaggagggtaatgaggaaggacgaacaccccacctccaggggttcgctaattttgtgaagaagcaaacatttaataaagtgaaatggtatttgggtgcccgctgccacatcgagaaagccaaaggaactgatcagcagaataaagaatattgcagtaaagaaggcaacttactgatggaatgtggagctcctagatctcaaggacaacggagtgacctgtctaccgctgtgagtaccttgcttgagagcgggagtctggtgaccgttgcagagcagcaccctgtaacgtttgtcagaaatttccgcgggctggctgaacttttgaaagtgagcgggaaaatgcagaagcgtgattggaagacgaatgtacacgtcattgtggggccacctgggtgtggcaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcggtatccattgactgtagagactaaaggtggaactgtaccttttttggcccgcagtattctgattaccagcaatcagaccccgttggaatggtactcctcggctgctgtcccagctgtagaagctctctatcggaggattacttccttggtattttggaagaatgctacagaacagtccacggaggaagggggccagttcgttaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatatactgttttcgaacgcagtgccgaggcctacatggtctacatttccagtagtttgtagtctcagccagagttgatttcttttgttattgggctggaagtaatcgattgtcgaatcaaggacaggtttgggtgtgaattaacgggagtggtaggaaaagggttgggttatggtatggcgggaggagtagtttacataggggtcataggttagggctgtgctctttgggaaaaagttatcatctagaataacagcactggatccaactcccctgtcaccctgggtgattggggagcagggccagaattcaaccttaacctttcttattctgtagtattcaaagggtatagagattttgttggtcccccctcccgggggaacaaagtcgtctattttaaatctcagcatgtctaccgcccaggagggcgttctgactgtggtagccttgacagtatatccgaaggtgcgggagaggcgggtgttgaagatgccatttttccttctccagcggtaacggtggcgggggtggaggaaccaggggcggcggcggaggatctggccaagatggctgcgggggcggtgtcttcgtctgcggtaacgcctccttggaaacgtcatagctgaaaacgaaagaagtgcgctgtaagtatt

SEQ ID NO: 155 - protein, 296 aa, Porcine circovirus 3
VRRESPKHRWCFTINNWTPTEWESIVECGGSIARYLIIGKEVGKSGTPHLQGYVNFKNKRRLSSVKRLPGFGRAHLEPARGSHKEASEYCKKEGDYLEIGEDSSSGTRSDLQAAARILTETSGNLTEVAEKMPAVFIRYGRGLRDFCGVMGLGKPRDFKTEVYVFIGPPGCGKTREACADAAARELQLYFKPRGPWWDGYNGEGAVILDDFYGWVPFDELLRIGDRYPLRVPVKGGFVNFVAKVLYITSNVVPEEWYSSENIRGKLEALFRRFTKVVFWGEGGVKKDMETVYPINY

SEQ ID NO: 156 - protein, 214 aa, Porcine circovirus 3
MRHRAIFRRRPRPRRRRRHRRRYARRRLFIRRPTAGTYYTKKYSTMNVISVGTPQNNKPWHANHFITRLNEWETAISFEYYKILKMKVTLSPVISPAQQTKTMFGHTAIDLDGAWTTNTWLQDDPYAESSTRKVMTSKKKHSRYFTPKPILAGTTSAHPGQSLFFFSRPTPWLNTYDPTVQWGALLWSIYVPEKTGMTDFYGTKEVWIRYKSVL

SEQ ID NO: 157 - protein, 2000 aa, Porcine circovirus 3
TAGTATTACCCGGCACCTCGGAACCCGGATCCACGGAGGTCTGTAGGGAGAAAAAGTGGTATCCCATTATGGATGCTCCGCACCGTGTGAGTGGATATACCGGGCAGTGGATGATGAAGCGGCCTCGTGTTTTGATGCCGCAGGACGGGGACTGGATAACTGAGTTTTTGTGGTGCTACGAGTGTCCTGAAGATAAGGACTTTTATTGTCATCCTATTCTAGGTCCGGAGGGAAAGCCCGAAACACAGGTGGTGTTTTACGATAAACAACTGGACCCCGACCGAGTGGGAATCTATTGTGGAGTGTGGAGGCAGTATAGCGAGATACCTTATTATCGGCAAAGAGGTTGGAAAAAGCGGTACCCCACACTTGCAAGGGTACGTGAATTTCAAGAACAAAAGGCGACTCAGCTCGGTGAAGCGCTTACCCGGATTTGGTCGGGCCCATCTGGAGCCGGCGAGGGGGAGCCACAAAGAGGCCAGCGAGTACTGCAAGAAAGAGGGGGATTACCTCGAGATTGGCGAAGATTCCTCTTCGGGTACCAGATCGGATCTTCAAGCAGCAGCTCGGATTCTGACGGAGACGTCGGGAAATCTGACTGAAGTTGCGGAGAAGATGCCTGCAGTATTTATACGCTATGGGCGGGGTTTGCGTGATTTTTGCGGGGTGATGGGGTTGGGTAAACCGCGTGATTTTAAAACTGAAGTTTATGTTTTTATTGGTCCTCCAGGATGCGGGAAAACGCGGGAAGCTTGTGCGGATGCGGCTGCGCGGGAATTGCAGTTGTATTTCAAGCCACGGGGGCCTTGGTGGGATGGTTATAATGGGGAGGGTGCTGTTATTCTGGATGATTTTTATGGGTGGGTTCCATTTGATGAATTGCTGAGAATTGGGGACAGGTACCCTCTGAGGGTTCCTGTTAAGGGTGGGTTTGTTAATTTTGTGGCTAAGGTATTATATATTACTAGTAATGTTGTACCGGAGGAGTGGTATTCATCGGAGAATATTCGTGGAAAGTTGGAGGCCTTGTTTAGGAGGTTCACTAAGGTTGTTTTTTGGGGGGAGGGGGGGGTAAAGAAAGACATGGAGACAGTGTATCCAATAAACTATTGATTTTATTTGCACTTGTGTACAATTATTGCGTTCGGGAGGGGGTATTTATTGGGTGGGTGGGTGGGCAGCCCCCTAGCCACGGCTTGTCGCCCCCACCGAAGCATGTGGGGGATGGGGTCCCCACATGCGAGGGCGTTTACCTGTGCCCGCACCCGAAGCGCAGCGGGAGCGCGCGCGAGGGGACACGGCTTGTCGCCACCGGAGGGGTCAGATTTATATTTATTTTCACTTAGAGAACGGACTTGTAACGAATCCAAACTTCTTTGGTGCCGTAGAAGTCTGTCATTCCAGTTTTTTCCGGGACATAAATGCTCCAAAGTAGTGCTCCCCATTGAACGGTGGGGTCATATGTGTTGAGCCATGGGGTGGGTCTGGAGAAAAAGAAGAGGCTTTGTCCTGGGTGAGCGCTGGTAGTTCCCGCCAGAATTGGTTTGGGGGTGAAGTAACGGCTGTGTTTTTTTTTAGAAGTCATAACTTTACGAGTGGAACTTTCCGCATAAGGGTCGTCTTGGAGCCAAGTGTTTGTGGTCCAGGCGCCGTCTAGATCTATGGCTGTGTGCCCGAACATAGTTTTTGTTTGCTGAGCTGGAGAAATTACAGGGCTGAGTGTAACTTTCATCTTTAGTATTTTGTAATATTCAAAGCTAATTGCAGTTTCCCATTCGTTTAGGCGGGTAATGAAGTGGTTGGCGTGCCAGGGCTTATTATTCTGAGGGGTTCCAACGGAAATGACGTTCATGGTGGAGTATTTCTTTGTGTAGTATGTGCCAGCTGTGGGCCTCCTAATGAATAGTCTTCTTCTGGCATAGCGCCTTCTGTGGCGTCGGCGTCTCCTTGGGCGGGGTCTTCTTCTGAATATAGCTCTGTGTCTCATTTTGGTGCCGGGC

SEQ ID NO: 158 - protein, 202 aa, Equid alphaherpesvirus 1
MRPEGVSRGRASSVSISMCPPPPNGARRASLGCAPPLNSRPVCCAPSSVSLSSSSSRRSMPSLGSSRSSSLPSTGSLRSITRDPERLPSRPPSYTAINPECLLERGAERPRAWTASVMTAPPSYSEALCQAPPAYELVPELSYHPTQDPRGVYSSRSDPHQTSRRRQNPICIFIIVVATMLLILGLLLTITLSSLTNGKKEK

SEQ ID NO: 159 - protein, 205 aa, Equid alphaherpesvirus 1
MDPAWRRRIHVSNPTTINVEPRPRRESLRRQLLSQLFFKKMSQLFKKKRQTRPSTVWYGRVAFPPSAWQGPARVSPYGYESDSENEEYTRISSATSSNVLTDSPTTTQDDPTGRCTDAYAESPAVLHLDDVTRNHRGAAALPSEVYLDGFCDQHKDPSGRMGPIKRRLYSKTFCRKFAAINIILLMLQILVLIGIVYRGFGKECM

SEQ ID NO: 160 - protein, 257 aa, Equid alphaherpesvirus 1
MGACCSSRRNRSPSLAALAEETEVVLRCLAGRVVDLPGGDEVRIAPDVGRPGQNFGYFKFPGPSRFAYVKFIGRAYALGSGRKFLLYLSRNFQVFGYEDGTGLHMLAKSLHDFLKFKGLSDRDLVVVDSVALTSQLRPLTLPIRSTSDVETLVAEEATTNYTSTENLLGQTQSSTHRPLGVPLSNVKTMGVPPTKPSSQRPRGKGGRPPARLKSIREETVSGMARAREECNSPSEHDRLTSEMTDCDSDSSVSSVFF

SEQ ID NO: 161 - protein, 200 aa, Equid alphaherpesvirus 1
MLPANRAEHSSDAEPRDIGSHGRNHGGCYSSDCKLGLLVDISNVVSPLPLDLTWSSWETTSQPARSRSYLNTRTYTIRACCDLQTRLHAFFIGVFEKRDPEKQILLPDLTNFKCILNNPRIMQELATEHSVCSAPFSAATQYDCDEDGEESTINGLCFHCHCKTPFSLECWQAANSAQAKILSVARGITSAKERQRENKR

SEQ ID NO: 162 - protein, 470 aa, Equid alphaherpesvirus 1
MALSSVSSCEPMEDEMSIMGSDTEDNFTGGDTCAEATRGLVNKSAFVPTQTVGTVSALRNVVGDPPKSVVVSFSASPQRAQPSNPKSERPAFGHGRRNRRRPFRRNNWKQQQRGWEKPEPENVPARQSAGSWPKRSSLPVHMRLGQRGGDSSSADSGHGGAGPSDRWRFKTRTQSVARVHRNRRRGNANHGSNTPGRSAGDRLNAAAASSIADVCRRVTSSRIGEMFHGARETLTTPVKNGGFRAENSSPWAPVLGFGSDQFNPEARRITWDTLVEHGVNLYKLFEVRSHAAEAARSLRDAVMRGENLLEALASADETLSWCKMIVTKNLPMRTRDPIISSSVALLDNLRLKLEPFMRCYLSSSGSPTLAELCDHQRLSDVACVPTFMFVMLARIARAVGSGAETVSRDALGPDGRVLADYVPGACLAGTLEAIDAHKRRCKADTCSLVSAYTLVPVYLHGKYFYCNQIF

SEQ ID NO: 163 - protein, 343 aa, Equid alphaherpesvirus 1
MLLGGRTAYLSVLGLITAYAAFTIWYTLTAQLHNPCVYATVSIDSKDGIAAKWEVYNSTIVYAYPENGAKRFSDGLSGFDYVCRENWVNESKLDVLKNMKELHDKVRIVVGTRNCRAYLWSVQLQMITGAWLIYIAFLCLRQERRLLGPFRNQNEFLSPTGYTFNYATYTLATTVLKTHYTKFALLLCEASLRRVALSRTFKRDPIGFLCEHSAALALIGLEVGTHFVARLLVVGTVTLVHTPCSQIYPIYLKLASWGFVVAVTIVEIVAIIYEKPPKTGSSANPPTPATHGVKGLCTSCCSTVLANLCGKLVYLLLVIGAVSILLHYEQRIQIGLLGESFSS

SEQ ID NO: 164 - protein, 1081 aa, Equid alphaherpesvirus 1
MAQRNPEPTIRVLYATDSCVITYSLMLLTGQESSEGVYAISYDWSSELDDLFGRQPRAPNTDAGDGWSSTEQSDADQLATALLQRKPSVSFCLLSGMVGGASDEPQDRVRPMFVCVFSTWTGARALAMTLSHGHPLSSNTLLQALTEEATFLLHNDLILALAITTENVTARSGRTAAAAKYDPQRGSVKAAVIGHSTGRSGLTSVYIHHENKVLTAFRRLYCNNNTTPFWFASKFGPGEREIVLATRYYLFQAIRCSRSGGTYDLQAVKDFIRTYNVPAAPNPTGLDLTHLTSFSLLSKFCCQSWYSRGPCALALPRYVDLRIQADVAEVSALEGFIAADRQGLRVSDREFITYIYLAHFESFNRKQLYDHLLAVSIADPGDIDRITSTSSLKRGTIEKFFAQVRIQLNIRDYIAHNVNPRVVCLPASIGSQYAQDKTYTPSSTTMSTGSAPLGVCDTSTPILKLLDRVESSLAGRGWIQTIVSPNKPQSVHSTPPLDQSRGDELSPGVSSQCGISRRLLHIASSPPVNGRALPLEVLFGQKGVPGPAPVYRVALPSKRQAFAVIANDRWETTTQNLARPGGSKQAYEGGFALAGFGEIDDCSLAWRDLQLTRTTSGVCRTALASSNASAQMYINRNEIFNSSLAVSNIILDVDFGIKRRVPLGMLHLAMRGFRAGIITTLSLIFSDATVQWDSYPCYFYKTSCPPQLVRALHRGEPSSFPDYVDGVEECYMESDFIDDYAAMEEYTDGPMDDYEMMMVDNECPQAACDNTPPNKEGGKTPLQRLDDTDACECTEKMGFRVTVPVPPPYLLAGPEALRGLARIIQQAVVLERTFTESMCSVLRDFSFIDTGVYSHGRSLRLPFFCKVGDSGEVYGGLYPFYVIPPKCNDVDEFVAQHSNPANFHFHAAPRHPTITHVITDLGGDYVSFFERKVARNREAIMTKRATLESLLSSANVSIKSHEAVEAFVVDVVLGEVVSHLATHFPDQSGEYQTVGVHTVVTKPDWILMQINRSGNAYRSQGFSCLRAKHLRSARGLARTFLSISADVHGRLCASISQQCFATKCGNNKMCTIFTLEVDRAK

SEQ ID NO: 165 - protein, 245 aa, Equid alphaherpesvirus 1
MFKWLMSSLCGIRNSTSPEVYEPIIGGQNPATMLRLQSALAAVNALLPATLTIEDVISSADNTRRLVKAQTLARTYQACQHNIECLSRHRASSDNPNLNAVVTTHMINAKRLSDTCLAALMHLYLSVGAVDATTDTMVDHAIRMTAENSVVMADVAVLEKTLGLDPQATVRAQDLLALNSGVLNSVNAVAEMTDPTDDVEFTQSVHSPLLPRQLSTTEVVGVPSPVKSNLKSKHKPKRKASLVAV

SEQ ID NO: 166 - protein, 326 aa, Equid alphaherpesvirus 1
MASVTNLVDSIVVVECGERWRARAEAAGRLLVLINNHTVELSGEHGSAGEFYSVLTDVGVRVACSSGYAIVLTQISGLLPVEPEPGNFSNVTFPENSAKYYTAYGIVDSGYRGVVKAVQFAPGINTSVPPGQMSLGLVLVKLARKSIHVTSIGSTRDGRTSEANLFYDYFAPKRVEDAGYDISAPEDATIDPDESHFVDLPIVFANSNPAVTPCIFGRSSMNRRGLIVLPTRWVAGRTCCFFILNVNKYPVSITKGQRVAQLLLTEDIDDALIPPTVNYDNPFPTYSPSESTKAPQSPVLWKFTTDFDREAPSSLRADGGFGSTGL

SEQ ID NO: 167 - protein, 100 aa, Equid alphaherpesvirus 1
MLSTRFVTLAILACLLVVLGLARGAGGDPGVKQRIDVAREEERRDFWHAACSGHGFPITTPSTAAILFYVSLLAVGVAVACQAYRAVLRIVTLEMLQHLH

SEQ ID NO: 168 - protein, 304 aa, Equid alphaherpesvirus 1
MSDTWRRRRSGCNDANATEELVYSTVRSDHRQRRPSRGTFVMRENDLYDKQSVSKENDLYESASPNDDKVYTRRGMSTAAHYRDSEHIYETCEGDEFYDACEYSLIGGGKLSTSNGRQSPAKAQPPPRGAAAAPPPRVPTRPPTRAAATSTTPRQQDCAPKQRASPGVNSIKSGKGLAFSGTPKTPKSQWYGATHLFNKNVFCAAVSRVAAAHASDAASALWDLNPPKTNEDLDRFLKAAAIRILVCEGAQLLEVANSTMESTPDGYAAAGPNGYDRRPRTASRRRSLKCKPPADDFFDDTNSG

SEQ ID NO: 169 - protein, 479 aa, Equid alphaherpesvirus 1
MCLLHISLPYLSCALLPGWYFDARPAASIVMFAAAEENDDPYPGKSGYNDTCELMDMDGAVASFDEGMLSAIESVYSIPTKKRLALPPPKAASPGALYQRLQGELGFPEGQTLLSAMEKWNEDMFSALPGHVDLYTEIALLSTSVDEVVRAGLDSLPTPSHYSPEVDLNAHGDEPFPEVPALEDDLEIYVISAQRFYLSELRTREEHYARLLRGYCVALLHYLYGSAKRQLRGSGSDASLMHKFKQVVRDRYYREAANLARLLYLHLYVSVTREVSWRLHASQVINQGVFVSLHYFWAQRRKFECLFHPVLFNHGVVILENDPLEFHDLQRINYRRRELGLPLIRAGLIEEENSPLEAEPLFSGKLPRTIGFLTHQIRTKMEAYSDAHPATPLFPLAEHSYSKRIGGRLSYGTTTEAMMDPPSPSAVLPGDPVPPLTVGVRQTAATLAIPSNLTLQSMETDGLDYSSMTGDELNQMFDI

SEQ ID NO: 170 - protein, 871 aa, Equid alphaherpesvirus 1
MDQHHGARGGAPIRRPRRSIESRSHPFRATGNTQRTYSTPRLSYRDGLSGRTASRDPQEQASNQDESSNPSTSNAQQSTSFWGYLRRVFSDDVPAQPQAPRPRADFAPPAGEESSSEEEEEEGPAQAPLDEEDQLMYADQYSVGDSSDENDEEEDPRLGSDYPTSAESSEYHDHGEMVAGAGAESESETDIDAEEEEEDDEDDEDDMEVIRDESYRLPRTWLDKSIRLMDEALAQSSELSKAITKSTRSLYDSQFAPGGRGYTQTATPSRRLVQLSRAGMYDSDKIVMTGDYMEVDDDPDSAYQSWVRAIRHPLAMNPSWEETISNHTNPSFSTDIDYDIDELIEKNLARTPPVFEGLLDSAEFFYKLPMLYTYATITQDEAYEERLAWSNTQALHGHEQSSWQALLVYYSRGGMYVSPTQEPRGIWRRALKQAMALQLKMCVLGLSDVVTKQNATHHHAAVTFLVDALLRTARNCYLASRLLVFAWERRRETGAKRPAEPLIALSGVTLLQPLPPEVSELLEQRTFDIGLRTPNSAVFRAFFGSLVYWAELRLALRDPASINCRYVGFHLQTSEIYLLARAHSASPGYTKEELVAMEAILTLATLMLEVALQWVHVACAQLLSENDTIKAFRRVSASIPHALAPLGSIRLHDAEFEVLSNPDVMVARDETALSQALFLGYFSVRTALTACMRDYSHEADGGSKETVTGVFLGVGLILQRLAGHLNFLLNCLAGAALYGGQKINIHSLTLPRYSLLADVMAPMLQRQSLVDFWRARDNMLEDLEITPRPGPPTQGKRVVVEMPLPSDDLPDMTPGASVNNGAGLGRMVDMAKQLQHYRETIIGEEATSSVGKRGLIRAGVGVAALRGRRRK

SEQ ID NO: 171 - protein, 747 aa, Equid alphaherpesvirus 1
MDGGGSSSWTHVSKNLIERRAVKGCLLPTPSDVMDAAVMALKDVTENIVGQQLFSVDRTNALSVIHTNEVPESIIATAIARDTSRDYLREYEGAAKCNLAATDLSHDEMWEVVIKRYWRYLRESSGAEVVDRGAVGQATQSVLSVLLLQSTFGKKRLSKNPFKHKGPNVGYKSNLEDLRSAFTKIEKYMYYMRPNDPMTKSEDTELRLHELLAYVTTCYRWLLWFMDLTDAKVLRNIDKGPVITHGPRESRPPDELVRRHLKSGPAISAGTGVALTLSTATADALIVLLRMSVSWTSHSWKSNTHGVTGAIVAAVELVTLIHHHLQYIINTVFAGYVCWLDGGVENSYLNSALRSQGRFDHFVGKLVPIMATLSWANMEKGTVMWFKYALAKSIVCHGSPTQHYLTVLESIASKRTGACPPQGSTFGRNPSGFPGQFCCPPQGPLPAPPNSKTRGTFRRCRPGSLRSSRQLPTSPPSNIVSPRTNPAIEGSTAAKNVQGAETIQVRSSGEFNDCIWYINGAYPHQRSDSSSSDNSTCSSTETQYITLPSTPSPTGDVVYTNPLLGPDEEVDASPQPVDPMSDYSAPKNPDYMRPRSTLVEEVWQLRDSDYTPYMRPSRAGRSRVRVEDQTLEPSSPAGCNPPANSPENDSDDAAVDSPPISPEVVYGTFRPRAKCVYDQYGLTALAALSASRAKARRTRPGPTQPDVCRERDEESAEPRHDGFIRRTMSTTGPPRKHPDQTERVSSL

SEQ ID NO: 172 - protein, 219 aa, Equid alphaherpesvirus 1
MEDYKLLQLETATVDAQAPPLPTKTVPVFAPPLSTPPQPNELVYTKRRRTKRKAKCRCLFFTMGMFALGVLMTTAILVSTFILTVPIGALRTAPCPAETFGLGDECVRPVLLNASSNTRNISGVGAVCEEYSEMAASNGTAGLIMSLLDCLNVGDSESVMNKLNLDDTQLAYCNVPSFAECYTKGFGVCYAARPLSPLGELIYKARQALRLDHIIPFPR

SEQ ID NO: 173 - protein, 468 aa, Equid alphaherpesvirus 1
MWLPNLVRFVAVAYLICAGAILTYASGASASSSQSTPATPTHTTPNLTTAHGAGSDNTTNANGTESTHSHETTITCTKSLISVPYYKSVDMNCTTSVGVNYSEYRLEIYLNQRTPFSGTPPGDEENYINHNATKDQTLLLFSTAERKKSRRGGQLGVIPDRLPKRQLFNLPLHTEGGTKFPLTIKSVDWRTAGIYVWSLYAKNGTLVNSTSVTVSTYNAPLLDLSVHPSLKGENYRATCVVASYFPHSSVKLRWYKNAREVDFTKYVTNASSVWVDGLITRISTVSIPVDPEEEYTPSLRCSIDWYRDEVSFARIAKAGTPSVFVAPTVSVSVEDGDAVCTAKCVPSTGVFVSWSVNDHLPGVPSQDMTTGVCPSHSGLVNMQSRRPLSEENGEREYSCIIEGYPDGLPMFSDTVVYDASPIVEDRPVLTSIIAVTCGAAALALVVLITAVCFYCSKPSQAPYKKSDF

SEQ ID NO: 174 - protein, 401 aa, Equid alphaherpesvirus 1
MMYQPDREPGEDSCLVLSSSSVQRCTGSQRGCMPCTWAASKAFVGIGLQACVLTSSILHIDLLTRNSTCLILMIISMYVLSLIRVPISKMETIVTVCRSIQALATLVAASVWVAGSAVKKEHLLIVVTVCILFVFIAGTQISLFYVICSANGTGTHFRASLLAIIGGCVLGVSVKLVELKDVPIGIGIAIAIIASCQDFGLALRDTCHYRIGRYACMRTFTDLGRGINYRWVTDVEAVPKIEEVAEEKVSLFKFFKEMPGVIFSPAVGTHATPIIWIVLRLVYGISNVWQTPAYVVFCLTVGHVSAMLLEQLVIRVNYTAEASSGIHSTAHAVCMVLAAFGYGVAAPLSLAFTVSGGILGALYLRKRATGARRLAATHISRWLIVCVYVAAGLCYATIITH

SEQ ID NO: 175 - protein, 405 aa, Equid alphaherpesvirus 1
MALPRAMRPGGSHPNNFLFNTLPVIDNPVERQRAMAVFERESLRDAFEMLTPIAPSLKNAFLIFNEDGLLIHTSVGGEQVYIPIQTNNMESYSWKKAPPAVFLANVDGRRGLLDAFKAKTQTNVSKVVFEIENYSPSRILTQTVFSARDQTEEDTEMGSDAEGATQTVSSRLVKHEFNNYALMLPTRQPDVSMSLSKAQLNKILGVCKQAGDPITFQCLFDDTLQVRSGDRQVVFSVDYQHADNCGVESSSSLLEKMPMKTKKSAPEPIRGISGRRLFTLVLDEDTNFKQLIQKLKLKNAGAVLNFFLDPDSIPMIGLSTKQPFSVMMFFMCSYPTQPCQVGFSPAAFSSTPMGAGVKRRASEEEESDQPPKKLFPDGKLFKSNFVLLMDKTGAKIPCPEQPMHF

SEQ ID NO: 176 - protein, 497 aa, Equid alphaherpesvirus 1
MGLFGLLKYAYSNRLVKHDAITTPPGIMTPIAIDLWNVMYTLMEKFDQERNFPLDGAAVTARCFFSLLRLLLKRSYYPIFVSDRGIYGDGRVKQGAKAIVSQTMSSYGGSGRLSSACFTGDEHDTEFQEDPEENDVSVPPQDTCPPTEISAGYVEPERKCEHSSTRWSALDGAPRLSYRLCVNLIRHLGYPYVNACNLEADDVCANLYHTNTVAQIYTTDTDLILMGCDIILDIMPLFPPTLRCCDVLMDLGVTYDEFLTEFVRCHTDLHEPQTLASVQSVISSLHSPPDEDEGADMPQTPSGHSWRCPNERRVISWRRQDDHDYDSSTEDSDQSDSSEEEEECPAGKGFGYRENPAVETCKRRTRPRRSAEASGRILHLKYTSRYPPIMESAPRALVRMAPPKTRHEVLERKFVKHVVSMLTPERRGSLSIMRRLPITQEPSNFSLVHDTLKNLVSEHEIARELANMFWNHIPTPTDYNTVLVNYWDDCGHRRQWS

SEQ ID NO: 177 - protein, 321 aa, Equid alphaherpesvirus 1
MSIENSKEAALTAELSLAGAFFYTPECPDIEHLRSLSVANRWLDTDLPISDDLKDVAKLTPAEREFYRFLFAFLSAADDLVNLNLGDLSALFTQKDILHYYIEQESIEVTHSRVYSAIQLMLFGNDAAARARYVASIIGDAAIGRKVAWLQAKVRECGSVAEKYILMILIEGLFFASSFASIAYLRTHNLFVVTCQSNDLISRDEAIHTRASCCIYNNYLGGFEKPEPKRIYELFSEAVNIECEFLLSHAPQYSHLLDIGAIISYVRYSADRLLGEIGLSPLFNAPKPSPSFPLAFMTVEKHTNFFERRSTAYSGTLINDL

SEQ ID NO: 178 - protein, 790 aa, Equid alphaherpesvirus 1
MALNFLQSDCPLAIIQDVISRVDAISDYGYANELSTTLPPRPSRSQVLEYITRVVDTLKPRCRVDERLYVVCGELVHLRIRTRNVEDLKYWLNSTEIALNEIVEKDILDHLDFIQRTLHAFESSEYRELCALGLQSALKYEEMYLAKMRGGRIESMGQFFLRLATTATHYTMEEPAMARVLVSGEVGWTYIFKAYFTALAGQVLIPATPIMLFGGRDCGSLASCYLLNPRVTDMNSAMLALMEEAGPILCNRGGIGLSLQRFNTPPKEGCSRGVMALLKLIDSMTMAINSDGERPTGVCVYFEPWHADIRAILNMRGMLARDETVRCDNIFACMWTPDLFFDRYQRYLDGESGVMWTLFDDTASHLCHMYGKEFEEEYERLEQCGFGVDSIPIQDMAFIIVRSAVMTGSPFLMFKDACNKHYHFDLRRKGAIMGSNLCTEIIQHADETQNGVCNLASINLPKCLAIPPPHTAGVPYFDFAALGRAAATATIFVNSMMRAGTYPTVKSQRGVDENRSLGLGIQGLHTAFLMLDLDMASPEARQLNKQIAERLLLNSMKASATLCRLGMKPFKGFEDSKYSLGELPFDSYPGVTLANRNAWRRLRTEIKQHGLYNSQFVAYMPTVSSSQVTESSEGFSPVYTNLFSKVTATGEVLRPNLLLMRTIRSIFPRECARLQALSTLEMAQWSVVGAFGDLPVGHPLSKFKTAFEYDQRTLIDMCADRAPFVDQSQSMSLFITEPADGKLPASKIMSLLVHAYKRGLKTGMYYCKIKKATNNGVFVGGDLVCTSCSL

SEQ ID NO: 179 - protein, 465 aa, Equid alphaherpesvirus 1
MNLGGNRFVQIGNGMSNIMYTDANGAVRWEQISPPAGFPQQQRGRGRGHVAFGLPNTLDWLPGFVQATPNSITISNMGGIQISSAGVITAAINSEQNSWMLSSFNPSLKLTRQVTLTDFCDPTAERPGLPIIRLRHHLDAIGSSPSSTPPGRNPQELDEAWAALSELSVSGRSDTTGLRPSLLSLTFLVASRSGEYSDKAAAEAVRAHVLANYRDRRTEQRLDRFGEYLQAMVRTHVFPHKHMTVFGGLISHVIQDKLASLTAVAGGVQEGARTNNSAVPRSSVYVPACAFLDVDHELKLGDASAKFVYLIFVYSQRLRREGVRVYVAVSKFDEVAFEDAVSFLFHKARTESAIRGTEGADAPEPHPNAALPLQELSSSRCEPRCPPSRLNNREFTNALYQWAPDLRGRPNRTSCMYAAYIRLGAIASDSPRTTRRSERFGSVDMPVVWLENVRWDPQDWVECSY

SEQ ID NO: 180 - protein, 1020 aa, Equid alphaherpesvirus 1
MAREHGSMRALVNSLAGLLGETDTEVPSLEPAMLMVLKSSISEFFLSTDTVSVDEAAELFPRLQFLACRAYAASHTPDAAMLAENLAGLVLWRIHQNWTDREMEAVDQMFVLLEIMNGESGVYMLSNNNLRISAKYGPSNMHLIVSTWLDTFRNVMSVAAKSTPDSLFNSKRMESIEEFSKPLVHAKFNLIYDMPFVQEGLRIVAKKINWILPFGLMVKGYKDMSMAPLTRALFLLSLVDSYFPKGTATEGSMKALTAYFRELVRTIDNSAFVPITEVNATPRTAYEVRVSSAIVHQNPYVTDTKAGMVAERVRTDAEILTSGALLSSGALSAHATAVAKLLSSNEPDDVSSRARARVAEHASNTWETIQASTTPTQVVEALVTAGFTSTHCGILERVVVDYFTRLRSTANSGPGRNDSLDYAQQVVGCVAIVGGVVFRLLLSYGFGLDYIRDYTTTISTLEPVYNELLSALGLADKGVEQTLKRSMAPRPYMNYISAARAALDDELLIVEKRTTGPGTHSAARESLLTWFDFRARDRWGVRIPDRDTTSTQVLAPITASLYSDDDLIAAASKLSFDALDAPPTQIIDDPSFAPYMLATVVLDAFNAILTSRFSADSVSQALRVLSWARDYGAGSIANVDGYRTKLTAIIASVSPFLQKDAPTPTMAHANNLEALLGELHSVVVAAIALIPERARMPVPERPSVKTSTFLAGLFLTAVYKRLETLVGHTAELTNNILGTASGIVSSIVTLNRFFNCRIMPVMGHYAVLIYPQSAQSAPFGRWRLVDVVDAVGSIYNEVSDLRADLRADVVTLKGDITSAAEALQECEALAVKTEGTRFGKLFNSLLTRHTQLARAQRGLAIRAGKLLGGSEAPGLKHVNTFLQRWGAISVMYQKATSGSTPEVNITSLANTLRHVWDEVQQERKATPPSRKFSNRDLGLAVERLMGGYPEVLDDDSNSTALTPKFNVDSWNSVNMDALRKRVTMPANIDSIRGNDSLATREYLKKEDLLAEIDAIFNNTK

SEQ ID NO: 181 - protein, 3421 aa, Equid alphaherpesvirus 1
MAQTLVPANKAGGAQADVVVIGYRNQYDSQLGEGSHVSCLRSSLSFLRLIFTHGIDFALTADSIDGVLVEGRAWTVAGSKSGEAPCMVSIVELPNKITYANSANALCCVFSRLYGDSGFYMHPGDGFQSTQIPARQFFDGVWKSRSESFALITIGAIGLAVYRHGDVAYVFDPHGHGSVTEAFVVRVLARDVYAYLTGYAATDPESDWAGALVFFVTCGPTESEPGFLISATSLLYGISETYLSDEQYVERSVATSHPGISTPPPLTDVAVGAVSEAWQYQELENGAATLDADMEGVAPAAAQVRASVIRQPTEKRVSLPKRRRPPWTPPTSSENLTTSGNTHTVAGRPSQKVRNATANVQNPTTGNGSAWAEALNDGGVDNASRPGQAVGAAGTLQNPAPGDALAMETTQASEEALRTRRVFRLSGEDEAPYDLGDAVGVLSAEINELATRAEELDVLSSTCVDSTVWVTRPHNSPDMDILEQFITMIFNRLLSFLVENGARTRTDSPSVIAGLFPGVLAAIPTQSAVVNLLQATGMALSDVASYKSILNMVSNEDSPVGELAVIKLELVASEVIKSTQKLVARVEELERDVTSGSVNPLGLYTYLTERLVAEMTKHGGDLFAREPKPGAVSLTERIGSLFRKARTREARATRTNASLARDLNAIEAAVHAAHDKFDAIEIKPADPSDTTNMDELAKSLDLSAVPTRVAKVIKKVESMVSDSIREYFLRGVQYSARAIAMDKTSGARFQVASAAVSNLERMLDSLPNFEKSLNSVVASAGIQGPPPAQISGSRKATLLGNLLRAGQNLTTDNALGAWAALLSEAHTEGHIERRELEAVIKEITSINDHAAKKASVEADMERFRVLSAAVDQATSDMYNSNPHALDTIIRGAEEMIRQAKVVEAHFDSGRISREAASRVGVRKREVETLANSARQRAAEISAARDEIYSRLQSLLLPLAGFVGLRAAPGVLEQLAKDAQRSTSEELRNLMHEAPKQVVSTVHSHLWSLFGQFREALEHPNSTTSSALAGVGPAFAIVVRSLLDPNKQRESVEFFITHADALADTVGAVEANPNSELAVAHAVNSIAAAIQTVSVGGRTITEFAFLVPMLERYQSRLTIVRETQRLATAQRAVAASVSAAAEVTTKLRAVAVPGVQEDVLKAAIAAAKHVSSEVTAAATAAERELARLDSKALSVAQVARAHQDLQKQTAVAKQRVGEIEEVLANLNKQQRELQDRAVHDRWKSDLLAALDKIETKSSFDVSELSRLRDLGAARGYDSREFAKRAEQALAANARAVIAVLDNVFKFNPYAPVNSKKETNPTISMLYNISWWDDFTLAAPILNTLFAGVDVEELMSLMRISTGMITFASTNGGRPKYNEAVNSLSSDMLKVPQLAKYVDFYGKWYTEFNAEMDVLSKLRADVLQAVGVRSGEISRALEEVTYVRNAEVAEKVLADGVKLYIPSDALIAKAVKYLEEFNQKRFAGSAFEEAIATTIRQDLSTAREAATQAEAARSEAMHRATHILREVVEAAKAADRDASANLANLKNLLRLTPPPQSVAAALDKATSSDDIVTQAALLLGTVESTPELDIKAVEWLQQARSIIDSHPLTTKIDGKGPMDPYAERIEKLHTLRGELDELRRQLTATEVSWDEAWGNFSRAVPRADVSMDGFVDAHQRARTLQASMGVISEMRADNKYGRLPPKVIGAIESKFAERHKNLETFNDTSTVLQTAITQFDSLVQQIPPEMEYDVLRSLLASFDQLAAVLPKWVGAEYAAYRSLLLMRIGLYDEYQKIAGIAAAGSRPHLEAVEYRSAVEDANLRRASRVSSLMGDKDVILSLREAKSSIDTAFPQVLLDAKGVPVEYRVCYRAVGDKLAAMLCGKLGVSMRPAMPSDPIVESSSVSGINVTHDILQLRFGLEKAYHSGFSTFARFVRHKRADWSPTEPAQAAAEIYAAVLATTLTREYGATWHRIRFMASSGLFVASPDSVCDTQGGRGKKSNNIVHLTLSDVVLSAMLRNSMHLVNFMRLDLTRQHEYMARTITPVLTKSLLSDILINTLVPTDTSTQWRSLPLAGDLEDLAQGMLFSIRMSDWKQNSFSTTSLLDVWMRSPGESGRAAAAKIASAIPGNPLATFTVLARMCIPPNALASLWEALQPEAFSQQNLSYDDVVTSRLDIASTVQTSVAVDPEMKSVDTKSRKQLYTTTGTSTTFTLAGSAPSAVKEVSALDVATCALMFGAPVVIAMETPEMFSEASGMSFCLKIFDSRPGATDHEIIQAVSSDLSSWGTSLLALDPNAIENACLTTQLEILSGLVASKLLAPAPPCLIVLDPSMRVIKVLWESESPPNDLVITLAEDEIIAELPYLNADDDLLPPMNPDDPIYTRVISGTNIPTATTEGSLFADQQLEFLRPESNPFPFASHDSSQSLDVPSSPSSGSDKYEEDPTGIVYDAPVDDMSDMAMNKAKAWQEWLEDGFAEDDYRELSNAMPAPPKTTPVVESKQKSDSVDRAPTLPPKAAPLPPSDASAIMSGKPVFKYTPGNKSAVPPSVPAPPTLPPAPPLPQSTSKAASGPPPTLPPAPPLPQSTSKAASGPPPTLPPAPPLPQSTSKAASGPPPTLPPAPPLPQSTSKAASGATQSDSGKTLTLDVPKTQSKDKVVPVPPTDKPSTTTPAALKQSDASKPPTAAIQHQQKLGTPVTPKDSGDKPTDNASAPVGVSPVTPDGTPGAKPPPKDAPPVDDTKQPVRKSLPSQVRGGRPYIRPSLGPFKFTGPPGYTIPVHGLPPSDSNVTQSTKEPPKPAVETPAAAPAKSAAAPAAAPAKSAAAPAAAPAKSAAAPAAAPAKSAAAPAAAPAKSAAAPAAAPAKDQTKSAAEVPKPAKDQAKDQAKDQAKDQAKDQAKDQAKSTTGQKLAKDPKSDGLTDDVALEIVPEKTPLPDDSPIGAVPENTPLPDDSPIGSPDLSASKNSHTTDAVSSDRFSVACKVPLPDSPEDDFYSYAVDVPLPDSPTDDPSSGRSDARAPTVGGVASIHRKSDSRNNRQSDAWRRAFADTLHGRPRNRSATKPCKSAPYKVPHAISYTKIPSVPNDQSGLAGKPCSEEPKRPTGRDTPVGSWNVSPSQAPADIPTAIPQNQNTSESPRTTSLKSPTRTVQSSMPADDIDELAEYDLQIARAVPVTKHPQPPPANQTPPPQEPPAPIDDRKNIRPPLSEEEIIAFLINMDDDDAGNASGPVDLHSVQAPKLPKQSKPTTNQFVPLDWWTETEPVVDADSLDLSPKQQRLFSWESTRDLLNINVRDRVYEEESDDEYTVSWDQHLVPAVSPTSVSSYSSDTVTDSYTDINDPRSVVCPLDGNAQNNVREFLDTHSSRVRVVPADELLSRRYFRSTSLSAMALLIAACRTIVRRLRATRRVLTDINRSLLLDLKQIRVLLG

SEQ ID NO: 182 - protein, 119 aa, Equid alphaherpesvirus 1
MAADKQQQQAPVAFNPADPPNIKAANFKDMLPVDVITILNQNIDELDYTKYTEDEISEGLKQLFMGTARTMVSLRQRHLKSLVRRSDMFAQNDASTWARPNIGLKRTFPPRFMQPISED

SEQ ID NO: 183 - protein, 275 aa, Equid alphaherpesvirus 1
MDSYNYRDFAVGGGLLQRIRLVVSGSLHCGESDATLNDPKHLPARCVFQFSGPDNNSVTFPIEYVLRLMKNWARSQCDPYIRIQNTGVSVLFQGFFFAPPNAPMASITSEHNNVILKSTHTTGLALSGIERVKRGGGLDLRPLQAMMQISCFTRMPVVQLSFRFMGPEDASRTQRLLERATSFGAMELHQKRTVDSCDRSNGIVSPREHRECRERQKRRPTPKRCASEVFASLASISSAFASERVKRRPVRIAAAILAFVFVAVILAIATKGRLF

SEQ ID NO: 184 - protein, 162 aa, Equid alphaherpesvirus 1
MYGRSNAPGCQPPVRAHPPSSPMTSRLQLAAMEQNASDAALSGMQQARSRQASPKRLRDLIPSSDVESMSLNALDDAYGHHDFSVWFEDLMPPEMELLMPTTDAKLNYLSFTRRLASSVFHSPDNRMPGGACDHTSALDARKERFATIINKFLDLHQILHDA

SEQ ID NO: 185 - protein, 620 aa, Equid alphaherpesvirus 1
MAASCNLDVIGEDGGCALTGGWQPGAFERPYMGFDARLLSTNSSLCSELIFSAHLMQISPTPQPREQVDVCEDPDNDPPEPSCAQFVDAVADSLALDKLCLICRTIDLYRRQFGLSPQWIADYAMLCTKTLAAPPCAVATVVAAFEFVYLMDKHYLRRGKTTLVGAFARRVLTLVDIQRHFFLHVCFRTDGGVPRCAASGTAPAATAMAGLGMADKVQYSNYSFLVQSSTRAMLLTVADVPSGDDGALQAVPHGRHGAGRPADGGGGVFGPKQQSTVAALMSWKECAKMIDCSGSERRRPGATMTCCERARADDDEYERQLLSTENTYLGSADNQAEGGNDTHLKWGYADLTLLLLSQSSTWEASEKTSLASQSRRACVEEYWASHRTVLARDTAPRFARFVDADAVPDTATGPVLATTLKHVRSRGRTCAECVLCNLILTREHWLALRRFKRDVISYSSNNANLFDCISPVLSALSDANSEPLAGDCGVGGGGTCPEDSGRFLELMHAAGTEAIYKHLFCDPMCALVELQTNPSVLFSPIGPPPEPDEIELQKARLASENWFSGRVCAGLWALAFTFKTYQIFTPKPTACAAFIKDAGLLLRRHNLPLISLEHTLCNYV

SEQ ID NO: 186 - protein, 326 aa, Equid alphaherpesvirus 1
MFDGRSDIYDSTSFAAELDDLYSCRSTGRENGRRSRVSTRGVHRDRCGSAAKRRSTKRRCELVARERDRYSLYLDYMASHPSDEISAVRELVVPLIKTTSITLPFDLNQTVADNCLSLSGMGYYLGIGGCCPTCTVSGEPRLHRADRAALILAYVQQLNNIYEYRGFLASVLAAAAQGDQAGVAASEGVQAERLLENVLAQPELFFAYHVLRDGGIQNVRVLFYRDLSVSGYMMYAVFPTKSVHLHYRLIDRLLAACPGYKIIAHVWQTAFVLVVRRDEGQQTDMDIPTVSAGDIYCKMCDLSFDGELLLEYKKLYAVFDDFLPPV

SEQ ID NO: 187 - protein, 1220 aa, Equid alphaherpesvirus 1
MAAREQANSVRRSGFFNPFIGKRPFFRPGSGQTAETERPRPPQHSYCTEVGSFKFIAPRCLDEEAPADQRRGVHVGTLERPPKVYCDGSEYDVLNFASGGCWPRRIRVWNGQDFRGDGFNPRFERFHVYDIVETSESASHDDPSRFAELSRPSGSVVTLLGMSECGKRVAVHVYGVRHYFYMAKAEVDSACGITTEAELVRAMVDCAHSSALSAALGNGNGGKQSGGSGGGWWGGKHVSADCFKVETVCHTTLYYFGSKPALYYRVSASSSRLGGFICDNFHPEITKFEGSVDVTTRLLLDNENFTSFGWYRLRPGTHGERVQLRPVERHVTSSDVEINCTPDNLEPIPDEAAWPDYKLMCFDIECKAGTGNEMAFPVATNQEDLVIQISCLLYSLATQNHEHTLLFSLGSCDISEEYSFACVQRGEPRPTVLEFDSEYELLVAFLTFLKQYSPEFATGYNIVNFDWAYIVNKVTSVYNIKLDGYGKFNKGGLFKVWDIATNHFQKKSKVKINGLISLDMYSVATEKLKLPSYKLDAVVGDVLGEHKIDLPYKEIPSYYAGGPDRRGVIGEYCIQDSRLVGKLFFKYLPHLELSAVAKLARITLTRVIFDGQQIRVYTCLLKLARERNFILPDNRRRFDSQADAASETSELAMDSQSHAFDSTDEPDGVDGTPDAAGSGATSENGGGKPGVGRAVGYQGAKVLDPVSGFHVDPVVVFDFASLYPSIIQAHNLCFTTLALDEVDLAGLQPSVDYSTFEVGDQKLFFVHAHIRESLLGILLRDWLAMRKAVRARIPTSTPEEAVLLDKQQSAIKVICNSVYGFTGVANGLLPCLRIAATVTTIGRDMLLKTRDYVHSRWATRELLEDNFPGAIGFRNHKPYSVRVIYGDTDSVFIKFVGLTYEGVSELGDAMSRQISADLFRAPIKLECEKTFQRLLLITKKKYIGVINGGKMLMKGVDLVRKNNCSFINLYARHLVDLLLYDEDVATAAAEVTDVPPAEWVGRPLPSGFDKFGRVLVEAYNRITAPNLDVREFVMTAELSRSPESYTNKRLPHLTVYFKLAMRNEELPSVKERIPYVIVAQTEAAEREAGVVNSMRGTAQNPVVTKTARPQPKRKLLVSDLAEDPTYVSENDVPLNTDYYFSHLLGTISVTFKALFGNDVRTTENLLKRFIPETPHKTPTKTQALLERAGFEKLTPFTPEEESRRILHTVFCTLEAAPHQS

SEQ ID NO: 188 - protein, 1209 aa, Equid alphaherpesvirus 1
MESAPKTVSLPVSPLGYVYARQKASLQTGTVSLTAARSVDSDLAVLPVIRGLTVEQTFTTNVAVVAGSKTTGLGGTGITLKLTPSHFNPNAFVFYGGSVIGASSNAPNLTRACEAARRRFGFSAFSSPPVENAVETSGEEICASLNLSPETTALYLVVTESFKEMVYVCNTFLHYGGTSTVTIDGQDAMKIPIYPVQLYMPDVNRLASEPFNAKHRSIGDEFVYSRPFFNSDLCRLLHGYVLGPAAVALRVRNLDGVARGAAHLALDENHEGSVLPQDVTFTLFDSTQGNAGKGSGRAQRQGDGSGSKNSASSGIERRLASVMAADTALSVDSIMGAGIYDTELPSVEDWPVLSSGDDTESLEALGAYAARLSGLVGAMVFSANSVLYMTEVDDGGPADGKDGSNPSYHRFYLIAAPYVAGNPQTDKDGRVLPHTADQQAAPINGSNQEFSLDYLALACGFCPQILARLLFYLERCDAGTFGGRNETDALRYLANTLESDVPCGLCNQATRPACAHTTLHRLRQRLPRFGAPVRAPIGIFGTMNSAYSDCDVLGNYASYGALKRPNDNEAPKSIMQDTYRATMERLVNELEQAKLIDKETLAQASPCSAPTSVVHDQASFIGLLSNIKDTIEGAAEQFMRTLVEARDFKIREGLADANHTMSISLDPYSSSFCPVTSFLARRTVFAVLQDLVLSQCHCLFYGQSVEGRNFRNQFQPVLRRRFLDMLNGGFITAKTVTVTVSDSGVLAPDLTRPASEPPTKDYDGDMARVSMEVLRDLRVKNRVLFSNGGANMSEAARARVAGMASAYRRPDKGSNILNGAVGFLVKQYHGVLFPRGHPPGIDTPNPQWFWTLLQRNQMPARLLSKEDIETITAIKRFSDEYSAINFINLTPNNIGELAQFYFANLVLKYCDHSQYFINGLTAIVVGSRRPRDPAAVLAWIDRTINGAADVEPAAQEVLQRLGSNPAAWTGTFTSTNMVRYVMDQRPMVVIGLSISKYNGSAGNNRVFQAGNWNGLNGGKNVCPLMAFDRTRRFVLACPRVGFTCEAGGFGTGVRENTLSEQVRGIVSEGGPMVQTAVFAAVLHALGARTQHLAVDDWIGLVDDEFLAASLDALNATVVDQFGEWSVEAAQELVKNMEAQTTAGAVAAGEGAFDFGACVGDTPQQSTSAFNGGLAMAAAPAGQKRSLPDDILFDMGAPPEKKSGLTFDML

SEQ ID NO: 189 - protein, 775 aa, Equid alphaherpesvirus 1
MEQDDAPAAMGSAQARQRLLAIFGQVQAYIFQVEMLKRCDPSALLPLVGSLKLNALTIRMLRRKLGGALIEQAQHQQTPLACALTMALEYAEVEGERVLRAVDDVNLAGPEGFFRATMRLDEPCEYHVRVHLDTYGGPIDAEVQFLHDAENFLKQLNYCHLITGFEAGLDALESVARFLTRTVGSGIVVPPELCDPTHPCSVCFEELCVTANQGEAVHRRLLECTCDHITRQMAVRVANIDIARHLPHALSVASERRAAAEAALRALEARRVQGHNGKSAGTEDPTQQVASRLLESHHVFKPASRCLYAVSELKFWLASTKHGDMGQPRAIDTFTENLETLDKQEKFFHLQAATVELALFGRTLDHFDRLFADQLLGLDVIDGMLVGSCAVSPDDHIEALIKACYTHHMSAPLLQRLTDPDTSNREALKQLLGRIGVDTDDGAGELGDALDVDLDNLGGAPPVNSTPCGEDALCRTVSEERPWDKLLERATADASQRRRMYAERLSKRSIASLGRCVREQRRELEKTLRVNVYGEVLLHTYVSSYNGFCARRGFCAAVSRAGTIIDNRSSTSAFDSHQFMKAALLRHPIDQSLMPSITHKFFELINGPVFDNAGHNFAQPPNTALYYSVENVGLLPHLKEELARFMITAAKGDWSISEFQRFYCFEGVTGVTATQRLAWKYIGELILAAAVFSSVFHCGEVRLLRADRTYPDSSGAQRCVSGIYITYEASCPLVAVLSAAPHGAIGAETVVIYDSDVFSLLYAVLQQLAPGSGAN

SEQ ID NO: 190 - protein, 980 aa, Equid alphaherpesvirus 1
MSSGCRSVGGSTWGNWRGDGGDLRQRRVLSPVCSAPAAGSWIGSQLGNVGNLLATPHPLGKPASSRVGTIVLACLLLFGSCVVRAVPTTPSPPTSTPTSMSTHSHGTVDPTLLPTETPDPLRLAVRESGILAEDGDFYTCPPPTGSTVVRIEPPRTCPKFDLGRNFTEGIAVIFKENIAPYKFRANVYYKDIVVTRVWKGYSHTSLSDRYNDRVPVSVEEIFGLIDSKGKCSSKAEYLRDNIMHHAYHDDEDEVELDLVPSKFATPGARAWQTTNDTTSYVGWMPWRHYTSTSVNCIVEEVEARSVYPYDSFALSTGDIVYASPFYGLRAAARIEHNSYAQERFRQVEGYRPRDLDSKLQAEEPVTKNFITTPHVTVSWNWTEKKVEACTLTKWKEVDELVRDEFRGSYRFTIRSISSTFISNTTQFKLESAPLTECVSKEAKEAIDSIYKKQYESTHVFSGDVEYYLARGGFLIAFRPMLSNELARLYLNELVRSNRTYDLKNLLNPNANNNNNTTRRRRSLLSVPEPQPTQDGVHREQILHRLHKRAVEATAGTDSSNVTAKQLELIKTTSSIEFAMLQFAYDHIQSHVNEMLSRIATAWCTLQNKERTLWNEMVKINPSAIVSATLDERVAARVLGDVIAITHCAKIEGNVYLQNSMRSMDSNTCYSRPPVTFTITKNANNRGSIEGQLGEENEIFTERKLIEPCALNQKRYFKFGKEYVYYENYTFVRKVPPTEIEVISTYVELNLTLLEDREFLPLEVYTRAELEDTGLLDYSEIQRRNQLHALRFYDIDSVVNVDNTAVIMQGIASFFKGLGKVGEAVGTLVLGAAGAVVSTVSGIASFLNNPFGGLAIGLLVIAGLVAAFFAYRYVMQIRSNPMKALYPITTKALKNKAKTSYGQNEEDDGSDFDEAKLEEAREMIKYMSMVSALEKQEKKAIKKNSGVGLIASNVSKLALRRRGPKYTRLQQNDTMENEKMV

SEQ ID NO: 191 - protein, 160 aa, Equid alphaherpesvirus 1
MDSPRGISTATGDAHAEAAVSPAAEIQIKTEAPDVDGPEATTECLDHTYTQQTSGGDGLDAIDTDDLLEMVLTSENTESEPGIPFALRGNFICCRDDNCRACRELPFRPSVIGFSRDPHVSMALDMTSGNWAYVPRVFPDTPTAPWMANYCIPDLDEHAD

SEQ ID NO: 192 - protein, 646 aa, Equid alphaherpesvirus 1
MDAYTVDGNAVSLPIYVAGYIALYDMGDGGELTLTRETVAAALPPASRLPINIDHRNGCVVGEVLSIVDDARGPFFLGIINCPQLGAVLATAAGPDFFGELSEGLSEQERLLYLVSNYLPSASLSSRRLGPDEEPDETLFAHVSLCVIGRRVGTIVTYDATPENAVAPFKRLSPSSREELLITAREAQSRLGDAATWHLSEDTLTRVLLSTAVNNMLLRNRWNLVARRRREAGIEGHTYLQASASFGITNGCNKADFCGAELVDTCGYKSGEKVHGAPYSRVTLGAKAFTSSSPNALPSSDNDKGGIGERTQKHISAMASSNPQTLSAAGAPLVSGDYILVPAAQYNQLVVGQHTSHPPINAGPAPVTHAVPSQYIPPAYNSLMPPSMYQAPPYWSVPHSANLEAQITALVGALAADRKATKGSDPHVIQGSQCSPPLSPQQERRYARKRRHDWDATTRDDLEGIYYPGERSPRPGERRAGRPSTTIADLMGAVSSLQQEVSQLRAIQTVTAQPQAAPAGLYKPIPAVPPQYSQYQYIQPQHAVSAIVAPQLPGIPSQPTQAVLAPQVPAGEAPGSAKVVAASTAPQQAEQARAAPQQFEAVTSAAVLPVTQPQASSQTVDASASTGLEFGRDDADIFVSQMMSAR

SEQ ID NO: 193 - protein, 329 aa, Equid alphaherpesvirus 1
MASSNPQTLSAAGAPLVSGDYILVPAAQYNQLVVGQHTSHPPINAGPAPVTHAVPSQYIPPAYNSLMPPSMYQAPPYWSVPHSANLEAQITALVGALAADRKATKGSDPHVIQGSQCSPPLSPQQERRYARKRRHDWDATTRDDLEGIYYPGERSPRPGERRAGRPSTTIADLMGAVSSLQQEVSQLRAIQTVTAQPQAAPAGLYKPIPAVPPQYSQYQYIQPQHAVSAIVAPQLPGIPSQPTQAVLAPQVPAGEAPGSAKVVAASTAPQQAEQARAAPQQFEAVTSAAVLPVTQPQASSQTVDASASTGLEFGRDDADIFVSQMMSAR

SEQ ID NO: 194 - protein, 587 aa, Equid alphaherpesvirus 1
MAEYVNYVLGSLYVSDTATSTIPTDVRNFIAPPFPLNFWSGPTFTVSSNTRADPLKLVAARHRAAAAAIDTLEAQSQYGVANVDALIRPLERQVAKVADALAALEDAARAAESADAATPQVNASEADQRPDNIGQSLSEIQIAKNDVPMEFDTNLAVDLLATVFVSRAAGGSNGVVFGTWYRALQDRLVTERPVATRSIDYRDGRMSKTFMTTAVVSLQSCGRLYIGNRPYSAFEAAVLCLHLAHRAVNSNYTYPTSFSGLIEQLPVYIEAFSTALGDGTLGKVGYEFNGARLPKNQFHVPGGGGRYERGALNGHGVLETLIRLKVLPAIPGSLGTTSTAVGPELDADQTAYIDDVNKAAAAFLVRAQNLFLTEDQTLLRSTINTITALLLLRRLLWNGNVYTDRLRNNFQLGAIVPNLAVSQRDARGASGGDAAAMVSRSGNNNFTFLCERYVSPIYIANREVELTQLFPGLAALCLDAQTVARDQPQHRAVNVSTGRNQTNLTRLIGIELENRRRTAPVPINEVLAAHDAVALQYERGLGLLMQKPRLRASLEETRRLGQFNVASDYDLLYFVCLGYIPSLTSAM

SEQ ID NO: 195 - protein, 272 aa, Equid alphaherpesvirus 1
MKRRQRLTARSRLRAGIRCHNRFYNAMVQDLASAKRNGVYGERLAPLFSELVPAETLKTALGVSLAFEVNLGQRRPDCVCTVQFGKGSDAKGVCILIELKTCRFSKNMNTASKNLQRKGGMRQLHDSCRLLARTLPPGSGEIVLAPVLVFVAQRGMRVLRVTRLSPQVVYSNAAVLSCTISRLAEYAPPVSAKSTRRRCVAKGTKAKAFSTKAAAEPVPSITPAQPSAAAAVVSLFPAAVPANTTNAAAVHQPVAVSHVNPLAWAASLFSPK

SEQ ID NO: 196 - protein, 352 aa, Equid alphaherpesvirus 1
MAARVPSGEARRSASGAPVRRQVTIVRIYLDGVYGIGKSTTGRVMASAASGGSPTLYFPEPMAYWRTLFEADVISGIYDTQNRKQQGDLAADDAASITAHYQSRFTTPYLILHDHTFGLFGGDSLQRGTRPDLTVVFDRHPVASAVCFPAARYLIGDMSMCALIAMVATLPREPQGGNIVVTTLNVDEHVRRLRTRARIGEQIDMKLIATLRNVYSMLANTSNFLRSGRVWRDGWGELPLSCETYKHRATQMDAFQERESPELSDTLFAMFKTPELLDDRGVILEVHAWALDALMLKLRNLSVFCADLSGTPRQCAATVESLIPLMSSTLSDSESASSLERAARTFNAEMGV

SEQ ID NO: 197 - protein, 848 aa, Equid alphaherpesvirus 1
MLQPYRKMLIFAVVTVAFAMAVWSTPVPATPSGVGNATWANNSFNITRYDKITMGQVYSNTSNSPIFFVVISERNFRIVNTPLGASVFWIPKGAMNPPQHQPCVANGPEPGDPRGPCVNSTVSLLFNENVEPFLMSKNLLEFEVLPDTYITGWTFERSKTATTKSNPVGVVLSPPRGSPSANTTIRDDGGPKKPLSIIDEYTTLVADLQNFTMTLTYISPFAAVWPIEAFQTGITVMGCDTTQVVAYLGHGFMGLQISSVNNPPLEMIVVPNDVSARILNRRPSRLRLEPPGPHAGPIYKVYVLSDGNFYLGHGMSRISREVAAYPEESLDYRYHLSLANLDTLAMLAELSSGKSTDVSYYMYRIVARLAVATFSLAEVIRLSDYMLLQEAIDVDMNLRLIVPLVMKYAAGGAADSSYTSSDVAMDQFDVAQSQIEKIVSDINVEAELRKPMYEHRSLLRSVYAYSRKPLPNAVALADRLILAMYKEAIKDRITWNSTMREVLFFAVGAAAGSHVILTDEPEPGAPAHKDASLFLSLNRNILLLCTAMCTASHAVSAGLKLEEVMAGLVAGGVQFSLLEVFSPCMASTRFDLAEEEHVLDLLSVIPPRLYTDLNTGFEDDGTTIHSYGRSANGILNSRIAYNFDAVSVFTPELASCSTKLPKVLVVLPIFTNRSYVITRTAPSIGLTYSLDGVNIAKPIVISYITYGNCEVSRATIKSGYLDNPGHTQTCVYCGSVFMRYMVSGAIMDLIYIDDKEVELQLVAGENSTIPAFNPKLYTPSMNALLMFPNGTVTLMSAFASYSSFKVPSTYLWASIGGLLLAILILYIIIKMLCGGVTNDGYKLLLSYE

SEQ ID NO: 198 - protein, 530 aa, Equid alphaherpesvirus 1
MDFKYSDTVIHNGVVFYITDGGHRVYFLYGGCLLSVPRPHATAESGEIAKFGLTLRGLTHNDRVVANYVRSELNRTGRHESAPSSEEDVFVDRLEVLAQGAQAFGRDICGSFDLEVYDPYLAECMVSLKVTSGLIVSTGRDIPQDGMLHLYTVPTITNASSGFIYTPNIACFTLVQAYLTELPPELETLISGLFDRIPVARPPLRDESGGHSRTDIIVTSPRAVKTMAIGGTTRCSKRPLRKTVVSDFVQVRLIPKPCSIWDSASRVASGASLQSLQLLFKIADEIILIEEPWPGLDEHLNQARSTIVDAILAVYGNEGKLRFFGGKLTQQGVTTLQRFVLCQFILGKWNLINCYAALEQLAESYIGAVPEARDPLPDPHLVADAVNEIIRESGILGELCEIIVRYTQPTDPVNGSGSEVVELEARLLAEFAANATRVELGLSSYDEVRNMEARIASVLNKLYAKDGIGGAAQVACRILGSGLPVAIVLNVSSITAFDGLDLSRKGAYYLYYLLSERLKRGGVTVHVSRK

SEQ ID NO: 199 - protein, 239 aa, Equid alphaherpesvirus 1
MPQVLMGNTRLHAPLEDGIPLIENDENSSQNEVDLYDYVSMSSYGGDNDFLISSAGGNITPENRPSFSAHVVLFAISALVIKPVCCFIFLNHYVITGSYDFAVAGGVCTVLYYMRLALTAWFMFRNIQSDMLPLNVWQQFVIGCMALGRTVAFMVVSYTTLFIRSELFFSMLAPNAGREYITPIIAHKLMPLISVRSAVCLVIISTAVYAADAICDTIGFTLPRMWMCILMRSSSVKRS

SEQ ID NO: 200 - protein, 1376 aa, Equid alphaherpesvirus 1
MDRRSEAFKIPVPEVIPAGQILSTIEVSSHRTLFDFFKQIRSDDNGLYAAQFDVLLGTYCNTLTLVRFLELGLSVSCVCTKFPELNYVNDGTIQFEVQQPMIARDGPHPVDQPTHTYMMKHIEQRSLSAAFAIAAEALGLIGGTTLDGTQISSSLRVRAIQQLARNVQTVLDSFERGTADQLLRVLLEKAPPLTLLAPLQIYRDEGRLASRVNRAVLVSELKRRVIEDTFFLTKHERNRKELVVARLAELVNCTAPSVAVTRMTHSDTKGRPVDGVVVTTAGVRQRLLQGILTLEDMAADVPVTYGEMMITGTNLVTALVMGKAVRNLDDVAHHLLGMQRDQVRANEKLIKDYEDVPSTARVRADLVLVGDRLVFLEALEKRVYQATNVPYPLVGNLDLTFIIPLGIFKPATDRYSRHAGSFTPTPGQPDPRTYPPQTVYFFNKDGNLVQLSFDSAAGTVCHSSFLDVDSVLVAIRREPHELHCAFGAYVTLPPAGTLLDQMRRFFERWHMLMPARPRWTAEALMTIDQLLSPGNANLRLELHPAFDFFVAPADVVIPGPFDMPNVMPTVMAMPRLINGNIPLPLCPVEFRDSRGFELSVDRHRLNPATVLAVRGAFRDANYPMVFYILEAVIHGSERTFCALARLIIQCIVSYWRNTHQVAFVNNFYMIMYINAYLGNGELPEECTAIYRDLLEHVQALRRLVAEYTVPGEAVGGQGHDALNNVLLDPALLPPLIWDCDPILHRADMGRARAQELWVDGVDYAAIPWVEMAEVNFGNTGGHLVHNRPIRGENKRNPIVPHHDPEWSVLSKIYYYAVVPAFSRGNCCTMGVRYDRVYPLVQTVVIPDLGAEEIAPTSPSDPRHPLNPRHLVPNTLNILFHNARVAVDTDALLLLQEVVTNMAERTTPVLATAAPDAGTATAVTQEMRTFDGTLHHGILMMAYQRNDETLLEGTFFYPAPVNALFACPEHLGALPGLNAEVLEAARDVPPVPHFFGGNYYATVRQPVAQHAVQSRADENTLTYALMAGYFKLGPIALSHQFATGFHPGFAFTVVRQDRFLTENILFAEKASESYFMGQLQVNRHEAVGGVNFVLTQPRANVDLGVGFTAAYAAAALRTPVTDMGNLPQNLYLTRGTIPMLDGDADAYLRRVVNTGNRLGPQGPRPIFGQLMPATPAGVAHGQAAVCEFIVTPVSADLNYFRRPCNPRGRSAGPVYACDGEADAVDVMYDHTQGDPAYPSRATVNPWASQRNSYGDRLYNGKYNLNGASPVYSPCFKFFTPTEVEAKGRNMTQLIADVGASVAPSTSNTEIQFKRPHGSTDLVEDPCSLFQEAYPLLSSTDTALLRTPHIGEIGADEGHFAQYLIRDESPLKGCFPRI

SEQ ID NO: 201 - protein, 314 aa, Equid alphaherpesvirus 1
MASAAFEIDILLPSDLSPADLSALQKCEGKLVFLTALRRRVMLSSVTLSSYYVNGAPPDTLSLMAAFRRRFPAIIQRVLPNKMIAAALGVAPLPPGAFIQNTGPFDLCNGDSVCALPPILDVEDKLRLGSVGEEILFPLTVPLAQARELIARLVARAVQALTPNAQAQRGAEVMFYNGRKYNVTPDLRHRDAVNGVARSLVLNMIFAMNEGSLVLLSLIPNLLTLGTQDGFVNAIIQMGSATREVGQLVHQQPVPQPQDGARRFCVYDALMSWISVASRLGDVVGGKPLVRICTFEGQATISRGEKAPVIQTLL

SEQ ID NO: 202 - protein, 734 aa, Equid alphaherpesvirus 1
MFGRVLGRETVQYFEALRREVQARRGAKNRAAEAQNGGEDDAKTAFLNFAIPTPQRHQTVVPGVGTLHDCCETAQIFASVARRLLFRSLSKWQSGEARERLDPASVEAYVDPKVRQALKTISFVEYSDDEARSCRNAYYSIMNTFDALRSSDAFHQVASFVARFSRLVDTSFNGADLDGDGQQASKRARVDVPTYGKQRGTLELFQKMILMHATYFIAAVILGDHADRIGAFLKMVFNTPEFSDATIRHFRQRATVFLVPRRHGKTWFLVPLIALALATFKGIKIGYTAHIRKATEPVFDEIGARLRQWFGNSPVDHVKGENISFSFPDGSKSTIVFASSHNTNGIRGQDFNLLFVDEANFIRPEAVQTIIGFLNQTNCKIIFVSSTNTGKASTSFLYNLKGAADDLLNVVTYICDEHMERVKAHTNATACSCYILNKPVFITMDGAMRNTAELFLPDSFMQEIIGGGNVSGAHRDEPVFTKTAQDRFLLYRPSTVANQDIMSSDLYVYVDPAFTTNAMASGTGVAVVGRYRSNWVVFGMEHFFLSALTGSSAELIARCVAQCLAQVFAIHKRPFDSVRVAVEGNSSQDAAVAIATNIQLELNTLRRADVVPMPGAVLFYHCTPHGSSVAYPFFLLQKQKTGAFDHFIKAFNSGSVLASQELVSNTVRLQTDPVEYLLTQMKNLTEVVTGTSETRVFTGKRNGASDDMLVALVMAVYLSSLPPTSDAFSSLPAQ

SEQ ID NO: 203 - protein, 706 aa, Equid alphaherpesvirus 1
MDAHIANETKHLLVHGNSKTRALVHIIVPDACLKKAGVDPVKLSDRHRASPSAAPVFRVFAQTRYHATGECSLWRTVFAGYVPSGAIVSALVPTVPADHPRLFQSTPDSGGLFVSLEIECDADGRFDAFTLVALRVDIADDPRTTEVLFTYDELLPPGTRYGADSKRVALLCRQFVAYVNSHPTVSQSAVTAASHIEAAVAEDVKSASGPQVSYGARIDPAEYLFSGGGFDNHQALARLEDDDKEIMSLIRRASEVIAKRNPVRVLSNPEVNGDAHRRQCVASGLRQGARGAHASDSHARVGFNSSIHDATALLLGLEPPDSGRFVNSGPQRHLPPQGPRSPASRDCQSGMLDDVLLLTPENSNPLTPLDWLDVGHAAVAGGDTPRDVWRRRPISLVARKHYGTCETFVVVSYENSTAWGGRRARDEHLAGSINPPVMQACVAAGVDHPRNLPPETRGELIAKFPMLTVPLGDTPPPVAAFDAAAELALIDHFRGACVSALLKAISERLRAEPRMSQLIEYDIPNNNRDCIISVAQRAPELLEAVALAIQNVTVTEFCNSALMLSALSHLNILSGNKRGRLPYHRSWLPSLAGGADAFLFDYYSSGGEVVKVSPVPLAILVTATRTGQHSCRFARGAPDSSSKTYERYLPGECYAYICVGLNRSFEALVVLPGGFACRASAARKLAWPAHLVEPILERYCWTIPSH

SEQ ID NO: 204 - protein, 370 aa, Equid alphaherpesvirus 1
MMAAASDSCLSLWEGSASSPNRQLTPEAVNCLTEALTEDVAVLRLIRSDPRVKIFMAVSVLTPRLARFAPPPPKLTHTAKCAVIMIYLTRPKALALQPKQFHMLVTFNKASVYSLVVRVKTKPFPVGTQRFRAVFQDPEFIGLPSDIPDPAAENIPTEINDRLDVSNFATPAQPPKDKYDCCVLAPGVWWSNANKAIYFLQMDVALLALCPAGWKARGLGIILGRLLNHQEGCATCRFTEHSDPLNATADSVATPESCLCWAPCLWRKAHQRELTVEGDRYLFRVLFMDAVERVRLTGLRRSPKITANLADLVVGIGPHGQQIPVNNAGWKLVALDADISRLIVCGCYALRYICPPTNSKHQPSSPDEYA

SEQ ID NO: 205 - protein, 317 aa, Equid alphaherpesvirus 1
MSFSARSRRQRLQLEEAYQREMIFKMHTLDLVREGVNKRSPAFVRAFTSAKEASLDLDRYMQAHSRVGRVEQNARALAQRVEAQAAVGEILDRHRRFLHPDFIDNFDSREDSIVEREERLGDVLSDINCDGGGGEVGDPQEWLGHEDEALLMRWMLEEAPRVSTRIAADPHSPRSTCPAPRKAPEDARCGARKPGEVNNYTPSAQPRSQETTVDHLASPDEGTRLGDRTRDLEHHSTAPMRTHPNVLASERRRLGVVHQREKSSESQESATRSKAIVGQEDQKWLGGIPPLSDEELQVDMGIPTMNGPIYPDYHRTA

SEQ ID NO: 206 - protein, 594 aa, Equid alphaherpesvirus 1
MARSRRRSSVDEMDVGGSATSEYENCGGPSFSPLNLSRPKKSTRGRSLRSAQAWGGKQLHPERSTPLARNDCGPSSKPRRRHEVGRSNKGLGASLDRTDEDTSQCPRIRASAIRCGASTRKIVRITGECDAQQGDSRPGRSEMAGWHSPPKRRRTPSRHGNSDNERSHLPRLSSHGVVRVGGRPLTQTPLQKTIILQPKLVRKVFMPTFTVNPEMHYRRVALGEIPKFGGAGSYGEVQIFKQTGLAIKTASSRSCFEHELAVSLLTGECSLRAQASLGIGGIICLMAFSLPSKQMVFPAYDADLNAYGYRLSRSGPPSVLVTESIERAFIGLGRALVYLNTSCGLTHLDVKGGNIFVNHSHFVISDCVIGDLSLMTLNTNSMAMRAEFEIDTGEEEIKTLRLPRSASQMTFSFVIGHGLNQPISVIADFINNSGLAKSTGPIKHDVGLTIDLYALGQALLELLLVGCISPCLSVPILRTATYYYYSNKLSVDYALDLLAYRCSLYPALFPTTPLTTIYGIPWDQVEGVFESIAGAHHREAFRAHLERYRLTHRRLFASIRIPSAFTGVLELVSLLCHANEKARLSIPLLWTPRP

SEQ ID NO: 207 - protein, 565 aa, Equid alphaherpesvirus 1
MDSSPVTYSGEPPYKLRRLSPSYPYVSKLRERCASKIETLSEGSARDSLEEEDVSEAMATGAFLATRLYLPSVLPQRITTLTFLDHFKKSRPLPNSDKRLNPIFYRLAYIRDLVGEMELEGIVERGTASRLLGASSPAGFVAGTYTHARDLSKTMSLASVRDAVLAIEAQTRDQSESQLWALLRRGLATASTMKWGALGPQYHPQWCEVSTNAKGIPNNPALQFGQTNERTARSLISALYVARSEAATPDLLVDPGCGQCFVFDESASVPGDAYACGLLMDARTGVVGASLDMLVCDRDPSGVLSPHSTQTTLDFFEIKCRAKYLFDPDLFSPVATAYANLLKHRTAVCLRKFLRSIKNPAVEYFAPTSVPGATEALITCNSSWKPREVNETNRRCGDFDRDHIALNLDASSDVWLFSEPDLESETITPARWDTGELALSVPVFANPRHPNFKQILVQAYVLSGHFPDHQLRPFLVTFIGRHRKRCEEGKTFTICDRPEGSPYNLNEVVHSSCAIPILLFVTPVIVDREGCWEDIEIESLTAFNKTADAIWDSDSPADVSEPTSS

SEQ ID NO: 208 - protein, 74 aa, Equid alphaherpesvirus 1
MGQRLSCGCFRTDQLVTHSGEVVSLNADTFEEFSMEEFDIPPPPPLPKPVFKQPGPYKIPARSQRCPSKRRDPY

SEQ ID NO: 209 - protein, 450 aa, Equid alphaherpesvirus 1
MARRGAAVAEEPLLPSSGIVGIGPIEGINWRTWLVQVFCFALTTSVLFITLVTASLPQTGYPCFYGSLVDYTQKNHSVVDGVWMRQIAGGVAPTLFLETTSLVAFLYYTTLVLVAISFYLIISAVLVRRYARGKECTAVAGCTRPTTTLIASHVTLVLGTLATWLLQVVILLLSHKQAVLGAAVYVVHFVSLVFFCMSFSGLGTASAQYSSNLRILKTNLPALHKMAGPGRAVMTNLGMGMLGISLPILSLMLGIILANSFHITLWQTVTVAVGVFVALGLMFLIIVELIVSHYVHVLVGPALAVLVASSTLAVATHSYFVHFHAMVSVQAPNLATASKAIVGIMAVISIIMLVVRLVRAIMFHKKRNTEFYGRVKTVSSKARRYANKVRGPRRNPQPLNVAESRGMLLAEDSETDAEEPIYDVVSEEFETEYYDDPQRVPERSHRREYR

SEQ ID NO: 210 - protein, 887 aa, Equid alphaherpesvirus 1
MPSIGPIPTIPDEGSRGSSATAAPRRAMASYRDTTLGGRAEGVAFSAVEDSYTSSVSLARMLYGGDLEEWVRHTRPGVSLEIQSRAPVRFPPPNNPSSRRVTVVRAPMGSGKTTALLKWLGEALDAPDISALVVSCRRSFTRTLAKRFNDAELPGFATYFTSTDYTMAGEPFRRLLVQIESLHRVDDNLLNNYDILVLDEVMSTIGQLYSPTMVHLNKVDALLTRLLKTCPRVIAMDATANAQLVDFLASARGERSVHVIINSFAAPGFSQRDGTLLRTLGTDVLRAALGFVLVDDENGTKVMETDSRPISARLREVNSAGFFGRLMDRLVAGRNVCVFSSTVSFSEIVARFCSQFTDSILVLNSLRPSEDVAFWGGVRVLIYTTVVTVGLSFDTAHFHSMFAYVKPMSHGPDMVSVYQSLGRVRELIHNELLVYVDSSGARAEPIFTPMLLNHVVSRQGGWPAEFSQVTDALCCQFKARCGPAYRTASTRGLALFVRFKYKHFFERCTLASVGDSINILYTLLESNQMRVAIEGCQFPLTAAGFCDFLQDLRLDAYAARKEIKQLRGPGGIAATPTEVFENDDVAVFIQKYLRPGVAHDEILALLVELNSPIVREQFVNVAVLGACLRLPAALESPEVFAGVYKHYASGVVPVISDAGALESVSITPDVNVLARWDLYKSCTRHARDLAWDPSRGGSGLDMSEDFITNTLSADYNRFQSLLVEIAKCNVTPLEMLAAGAVRGVTTALSGRPKSRVPLSKGEHAVSLFKVLWEDVFGAKLAKSTQTFPGGVRVKNLRKDEIVALLESVNVNHSECKTHRELYALLMCNRKLFAGPRYKLRAPKWSRNLCFLELDNTGTCKTPLDAALADLAPSAWPQVYGAVDFDAL

SEQ ID NO: 211 - protein, 716 aa, Equid alphaherpesvirus 1
MLCRRGSDYTAEFCHVPVSGELLKRGARDASLVTPARVASAAQTAAVPGCWPLAPLGNAMLWKSVYGGITAALKRAVGSFAFYQPLVLGINTQTGLLVTLRPAASAGEGGGDHVSPRAAIVNVSVEVDLDPAGIEASAASSTGSSLARARLCTLRDGYFLSKRDIALEVEIATKEVSFYRKYDSVQQPANKRRGDMADLFVVHERTLLLGGCKRMGVKVLLPRTFDCLVASSQSVSGLAAMALYKQWHATLFSVELPDTVVQIFAYLGPELNPCGEEVDYCCFVGFPGLPTLKASSSTTEAVRDAMAAYRLSDGLWPALGMSAFHFLAPWDPEDRWPGESEAKRVEGAVHRLQLGTEDDWGAGRVSCILESDAVMQGPWFAKFDFSAFFPTLYLLLFPANERLAEVVRLRARGQHPTLKLALVSFFGGLQHINPVAYRSIIALSNGISKRLEHEVNQRGFAICTYVKDGFWGAAGNLPSDSVSYADALVYAEELRSAAQKAALGHVSEMGFSLPEGVHLNLRLEGLFTDAISWSTHCYWLYNRFTKMEDFVGFPAKSGAGRAAKASLSALLPLVAAVCDSSDMSTLHQSVRGACEQLVAGAFAERNNPQFWSTRTGIESSTLLPPAVYRNGSLLDRDCGQREIVLTRKHDCESPSPVPWTLFPPPLVLGRIDCMVYLTSIFKTYLSMLNRAISASCDADESMNVDFPISDYAFLFT

SEQ ID NO: 212 - protein, 303 aa, Equid alphaherpesvirus 1
MSATMRAEIDPLAHSATVDEMVEAITKGDDSVTTIVDDLVWHATPRFVTEVREVPGLPPSFTTTSVTDMRVDASSEKLMLTLDGQEGSEISCETYMRSCLDLPAFKGFSLFVVTPLEDRVNVLGVAPVILSHRLVLYRPTDLIDFTLCIIQMYLENCSTKRATSSLFVQIECILRNISKTITPLLKMRRLMYIGATWTLNTLMCVTNHNPFDQARVLPNYMMAKMLLGQKSNTPAILDAIYSAGYRQTLSKRPITPCPSGVLRCNKAHLNAPLCTKVVADTLYSWWTATDEKPVPESIYVLYD

SEQ ID NO: 213 - protein, 753 aa, Equid alphaherpesvirus 1
MSADSIEPKQKRLRYADANKGRKVERKNTPRFEATTGLQSPGEEEQISADGGWVLIHPTPKTMLFKEILMGELGYTEGQGVYNAIRSTEAAIRQIQTTILTNTLNATRYEDLAKDWQTHLDSRGVSAEEIAATYGMYSEGEAVRVAEQIFATWHRTLQMSLLDFVRSITACFSASEPDGTASFAKYIDWIACLGLIPLQRLKRAPGATVHPKLWRKLPTDVPSLESCVDERDLAGKLYVANSLLREGLEAVVELARCTASVAIMDYDRVNIFYHYTRREVVAIDSTTGKRGECLVLWQPIWKDGSVLFDSPLQRICGEVCNCHALREHAKLCQLLNTVPVKILVGRKKDEAQGPGWASKAVDKLMGEGEELHSSSAASRLVKLIVNMKSMRHIGDITETVRSYLNETSTNLLSGAQVDTSLPGFGQSGKTKQGGNMPVQEAFRTSVINGINGMLEGYVNNLFKTIEDLRTGNSGLLDQLRDRESEITHLREQLLRVSQAAADGSTQPGASSAALPGSGAKSGAGGLGHEVIDIRNLMGDDGYVANSFQSRYIPAYTADMERLSRLWDQELLRCFKMNRITNNQGQEMSVSYSNSSISLLLAPYFFSILRARHLGFLITHQEAYRSEEELCVAVFKKTRLEAYLTELSTLFVARVRNSIAALNSTKRDLPVNDNEAQSDEEQLGKPSDERYYEGRDRSASPQRDRGRNGRGYHKRRRFSNNYRRRSGLARDSSIRDRSQRGSRPTPLLHDHVGH

SEQ ID NO: 214 - protein, 881 aa, Equid alphaherpesvirus 1
MESADILPGSRGTVDRRCEGSEEKITPPRPVEDFNPQLFPNEVYLNFTSMHGIQPVVARIRELSRKTVSAAMVPPLEWFERLPRLETPLDIEPLHLPFSVYLISGNAGSGKSTCIQTLNETMDCVITGATRVAAQNVYTKLSSAFATRHINTIFQEFGFRGNHVQAQLGKYQYSCSSSPPPIEELQKRDIVYYWEVLVDITRRLFESTASRGEFENIRALERLLGRAPGSLTRLAFCTNGSLPAFTRTNIVIIDEAGLLGRHLLTVVVYCWWMLNAAYKSPQYAEGKVPVIVCVGSPTQTDSLESRFEHKNLKCHVRSSENVLTHIITNRTIREYVSLSTNWAIFINNKRCQEYEFGELMKVLEYGLPITEEHMRLVDTFVVPEAYINNPANLPGWTRLYSSHKEVSAYMAKLHAHLKVSGERQFVVFTLPAYTFVKTAAFDEYKKITQQPSLSLDKWLAANASRVSNYSQSRDQDAGKTQCEYYSEHGVVVARTDVTYVLNSQVSVTTRMRKFVFGFSGTFETFDAVLKDDAFIKTQGETSVEYAYRFLSTLLFSGMINFYNFLKRPGLDEGRVREAYRRMAALTAKLIPGASVLESACDNPSGAPLNFRGLTDPPGFTGGTTNDWDDDNDVVFAALNEGAIDMLYCNYEFVRPETTQEVYSQFLMLKTMFVGRYSIFMDLFGGDFESSPFDTFVDNISYKGCEIFVGSMRGGVSSIALQTDSYTLMGYTSAPVYPFVEELARRKLHEGIAELFGAMNMPRMVLRDQHGFMSVLNVNLSEFVESVDDVELDMATAVDYGLSSKLAMTIARSQGLSLDKVAICFPRNNLRINSVYVAMSRTVSSRFLRMNLNPLRERHERDTVISEHILAALRDRDVQIVY

SEQ ID NO: 215 - protein, 225 aa, Equid alphaherpesvirus 1
METCSPPVTFITYALYGIKTSPAWTLPNFEQVICSCDWGYRLIAVGAESKCDVTPQGSFVIQHGASITALVLDCGVEFCSYAFTHAENTRVPLTTEDGSVLVVPFCGWVCVGRDRCLRSMSGGVLTISWDTSQTAYISVAVYRPPTLQCHALDCTRAETTVCSTAAITDASESDPLYADQEGDQTQDQDGGHDFLETILMESDLYGTNGASALLEPCFPCLSNND

SEQ ID NO: 216 - protein, 179 aa, Equid alphaherpesvirus 1
MDVFGRGRAATADDYRRFLERNSRAAAKLAAATTPTHTASSRQQPEVSARPRHRSLSSRRNSVPHYDPHAGAGGDGQLALSERPSGTLGALAMAAQRRKSIGRPDTPVNAAMGSALRSSQRPRGDVRNPRGEGQTQRGGSRGEANHAQRRQSVTQSTAARQTQPHQGRPRPRRNTLRHM

SEQ ID NO: 217 - protein, 212 aa, Equid alphaherpesvirus 1
MESALTVLSGWGWPVEVVTGPVADHLTEMRPPAPTGTTCTTTSTPTPLCVPDLSVESIKGLAPDGENANYVGFDTMFMVSSIDELGRRQLTDTIRKDLRVTLAKFTIACTKTSSFSSASSTRRRKRHCPSSERVMRSNKSLQMFVLCRRAHAKQIRDQLQSVIQARKPRKYYTRSSDGRTHPVVPVYIYEFSAVDKVYLHRDNVIEADAQAK

SEQ ID NO: 218 - protein, 312 aa, Equid alphaherpesvirus 1
MSSACDHETEASHVNIPETTPEENGSNSSTPTSEIGPACVVSPAPGETGAPPPKRRRPCGLPQGVALINTSVSTHPLFTTSCQSSWEDVEREFNIAPSWRPILEREMQQPYVRLLLNEYKLRCASEEVFPPKEDIFAWTRFSPPEKVRVVIVGQDPYHAPGQAHGLAFSVRKGVPVPPSLRNIYSAVQKSYPSFRHPMHGFLERWAEQGVLLINTTLTVARGKPGSHATLGWHRLVRAVIDRLCTTSQGLVFMLWGAHAQKSCSPNRQHHLVLTYGHPSPLSRVNFRDCPHFLEANAYLTKTGRKPVDWQIE

SEQ ID NO: 219 - protein, 218 aa, Equid alphaherpesvirus 1
MYQILIGCVWQKSPYINQCTEFQPPLSFVTPERMRRFMRCWARLELVYMLAWIVTTKLVKATRLDFTWGPGEPKRILEASCGSGPIMKGQLFTSPNIKNLLNRTTGIMVKAHCNPPEAILWVDTPPKPVWVNPFAVVQGLAEDVTNGNMPQDFKEKLLFALDDSLSQSQSSPDEILGPPPLGCFTGPFFLSPPKSKDIAEGLKDSCIPASYYANLQKT

SEQ ID NO: 220 - protein, 218 aa, Equid alphaherpesvirus 1; identical to SEQ ID NO: 219

SEQ ID NO: 221 - protein, 1487 aa, Equid alphaherpesvirus 1
MASQRSDFAPDLYDFIESNDFGEDPLIRAASAAEEGFTQPAAPDLLYGSQNMFGVDDAPLSTPAVVIPPPSPTPEPRGGKAKRSPSAAGSGGPPTPAAAAQPASPAPSPAPGLAAMLKMVHSSVAPGNGRRATGSSSPGGGDAADPVALDSDTETCPGSPQPEFPSSASPGGGSPAPRVRSISISSSSSSSSSMDEDDQADGAGASSSSSSSSDDSDSDEGGEEETPRPRHSQNAAKTPSAAGSPGPSSGGDRPAAGAATPKSCRSGAASPGAPAPAPASAPAPSRPGGGLLPPGARILEYLEGVREANLAKTLERPEPPAGMASPPGRSPHRLPKDQRPKSALAGASKRKRANPRPIPQTQTQAPAEEAPQTAVWDLLDMNSSQATGAAAAAASAPAAASCAPGVYQREPLLTPSGDPWPGSDPPPMGRVRYGGTGDSRDGLWDDPEIVLAASRYAEAQAPVPVFVPEMGDSTKQYNALVRMVFESREAMSWLQNSKLSGQDQNLAQFCQKFIHAPRGHGSFITGSVANPLPHIGDAMAAGNALWALPHAAASVAMSRRYDRTQKSFILQSLRRAYADMAYPRDEAGRPDSLAAVAGYPAQAAAAAASQQQPEAPAPSVRVREAYTRVCAALGPRRKAAAAAAAPGTRAPRPSAFRLRELGDACVLACQAVFEALLRLRGGASAVPGLDPSEIPSPACPPEALCSNPAGLETAALSLYELRDLVERARLLGDSDPTHRLGSDELRLAVRAVLVVARTVAPLVRYNAEGARARASAWTVTQAVFSIPSLVGGMLGEAVSLLAPPTRSQQPSSSSPGGEPFSGSAAAEGSLQTLPPLWPTVPGKQSATVPSSHSQSPQHSQSGGGAGATTATCCRATQTNARSRGQQHQPQKARSPQAAASPAHLSQEAMPGSSSDDRAIHGRPRGKSGKRRSEPLEPAAQAGASASFSSSARGYDPSGPVDSPPAPKRRVATPGHQAPRALGPMPAEGPDRRGGFRRVPRGDCHTPRPSDAACAAYCPPELVAELIDNQLFPEAWRPALTFDPQALATIAARCSGPPARDGARFGELAASGPLRRRAAWMHQIPDPEDVKVVVLYSPLQDEDLLGGLPASRPGGSRREPLWSDLKGGLSALLAALGNRILTKRSHAWAGNWTGAPDVSALNAQGVLLLSTGDLAFTGCVEYLCLRLGSARRKLLVLDAVSTEDWPQDGPAISQYHIYMRAALTPRVACAVRWPRERHLSRAVLTSSTLFGPGLFARAEAAFARLYPDSAPLRLCRSSNVAYTVDTRAGERTRVPLAPREYRQRVLPDYDGCKDMRAQAEGLGFHDPDFEEGAAQSHRAANRWGLGAWLRPVYLACGRRGAGAVEPSELLIPELLSEFCRVALLEPDAEAEPLVLPITEAPRRRAPRVDWEPGFGSRSTSVLHMGATELCLPEPDDELEIDGAGDVELVVEHPGPSPGVAQALRRAPIKIEVVSDDEDGGDWCNPYLS

SEQ ID NO: 222 - protein, 293 aa, Equid alphaherpesvirus 1
MPHGQPCGACDGSCRMAQRGTPSTSPLIPSLTPSPPAGDPSPRSSQRIDAVRVPARLPGGSDHPEYGMPLSPRALRPYLARGPGAFCAPPWRPDVNRLAGDVNRLFRGISTSSIHVTEDSRTLRRALLDFYAMGYTHTRPTLECWQSLLQLLPEQSFPLRATLRALNSEDRYEQRFLEPPSDPPNTLFGEECDVSGDESPSEEEEEDEASGESSVSEFSPEEETASSEYDSFSDVGEDDSSCTGKWSSSESESDSESDAPTNNHHPTTRASAAKKRRKRQPPKGERPTKSARR

SEQ ID NO: 223 - protein, 236 aa, Equid alphaherpesvirus 1
MDGAYGHVHNGSPMAVDGEESGAGTGTGAGADGLYPTSTDTAAHAVSLPRSVGDFAAVVRAVSAEAADALRSGAGPPAEAWPRVYRMFCDMFGRYAASPMPVFHSADPLRRAVGRYLVDLGAAPVETHAELSGRMLFCAYWCCLGHAFACSRPQMYERACARFFETRLGIGETPPADAERYWAALLNMAGAEPELFPRHAAAAAYLRARGRKLPLQLPSAHRTAKTVAVTGQSINF

SEQ ID NO: 224 - protein, 272 aa, Equid alphaherpesvirus 1
MNSDMMTAATAGTEVFRCALARRRNANPPHLVLAPTFAAAAAGGAANSSGEEAPRGERKHLFNPFGCMLGRSYFRRCREEMNEGYFAKVPTGYFPVAPSEVPCRVPVEGVVAGEVLSYSALPLPKIEKRFYKQLNDGTFVRLPFLYPEVYYEGEEEPADERYYIRADAADASSADPSTLPEEAFAKVPPAIAEGITNWQGPKRIPIPSERYVMKLGFEYQLHVTEDAFQEVNTSFMRLDLQSSPDPHPRGARQPRSRAHVSAENPEDTPVAV

SEQ ID NO: 225 - protein, 418 aa, Equid alphaherpesvirus 1
MGVVLITVVTVVDRHKALPNSSIDVDGHLWEFLSRQCFVLASEPLGIPIVVRSADLYRFSSSLLTLPKACRPIVRTRGATAIALDRNGVVYHEDRMGVSIEWLSVLSGYNHLNSSLIINQPYHLWVLGAADLCKPVFDLIPGPKRMVYAEIADEFHKSWQPPFVCGKLFETIPWTTVEHNHPLKLRAAGGEDTVVGECGFSKHSSNSLVRPPTVKRVIYAVVDPARLREIPAPGRPLPRRRPSEGGDARPEAALARSRARSVHGRRRDAAPPRGPAGARRPPGGRRDVDGTPTLGSVRPDIHTLKGRGLSPVPHLALWVEQWFLALQKPRLYTHHRLALHNIPSILKGEKREDTFEDNRRDELRHDDSRHRRHRGLPLRARSPPQCQPAPPRLGPHLRRSRGRGGRQLQRRGGAERRA

SEQ ID NO: 226 - protein, 382 aa, Equid alphaherpesvirus 1
MENKQCDHLTDWFSTTSDASESMDTTPPLPPPTPSVDPSYSGAAADEDLYSDISEGDLEYSDCDSASESDEDDDDCLIPSKEKAREVAASFGYTVIKTLTPGSEGRVMVATKDGQPEPVVLKIGQKGTTLIEAMMLRNVNHPSVIQMKDTLVSGAITCMVLPHYSSDLYTFLTKESRRIPIDQALIIEKQILEGLRYLHAQRIIHRDVKTENIFINSVDQVCIADFGAAQFPVVEPADLGLAGTVETNAPEVLARAKYNSKADIWSAGIVLFEMLAYPSTLFEDPPSTPEEYVKSCHSQLLKIISTLKINPEEFPRDPGSRLVRGYIEYSRLERKPYTRYPCFQRVNLHIDGEFLVHKMLAFNAAMRPSAEELLSYPMFAQL

SEQ ID NO: 227 - protein, 411 aa, Equid alphaherpesvirus 1
MLTVLAALSLLSLLTSATGRLAPDELCYAEPRRTGSPPNTQPERPPVIFEPPTIAIKAESKGCELILLDPPIDVSYRREDKVNASIAWFFDFGACRMPIAYREYYGCIGNAVPSPETCDAYSFTLIRTEGIVEFTIVNMSLLFQPGIYDSGNFIYSVLLDYHIFTGRVTLEVEKDTNYPCGMIHGLTAYGNINVDETMDNASPHPRAVGCFPEPIDNEAWANVTFTELGIPDPNSFLDDEGDYPNISDCHSWESYTYPNTLRQATGPQTLLVGAVGLRILAQAWKFVGDETYDTIRAEAKNLETHVPSSAAESSLENQSTQEESNSPEVAHLRSVNSDDSTHTGGASNGIQDCDSQLKTVYACLALIGLGTCAMIGLIVYICVLRSKLSSRNFSRAQNVKHRNYQRLEYVA

SEQ ID NO: 228 - protein, 797 aa, Equid alphaherpesvirus 1
MGFIYARKLLLCMAVSIYAIGSTTTTETTTSSSSTSGSGQSTSSGTTNSSSSPTTSPPTTSSSPPTSTHTSSPSSTSTQSSSTAATSSSAPSTASSTTSIPTSTSTETTTTTPTASTTTPTTTTAAPTTAATTTAVTTAASTSAETTTATATATSTPTTTTPTSTTTTTATTTVPTTASTTTDTTTAATTTAATTTAATTTAATTTAATTTAATTTAATTTAATTSSATTAATTTAATTTAATTTAATTTAATTTAATTTGSPTSGSTSTTGASTSTPSASTATSATPTSTSTSAAATTSTPTPTSAATSAESTTEAPTSTPTTDTTTPSEATTATTSPESTTVSASTTSATTTAFTTESHTSPDSSTGSTSTAEPSSTFTLTPSTATPSTDQFTGSSASTESDSTDSSTVPTTGTESITESSSTTEASTNLGSSTYESTEALETPDGNTTSGNTTPSPSPRTPSFADTQQTPDNGVSTQHTTINDHTTANAQKHAGHHRGRAGGRRGSPQGGSHTTPHPDRLTPSPDDTYDDDTNHPNGRNNSIEIVPQLPPDRPIIELGVATLRKNFMEASCTVETNSGLAIFWKIGNASVDAFNRGTTHTRLMRNGVPVYALVSTLRVPWLNVIPLTKITCAACPTNLVAGDGVDLNSCTTKSTTIPCPGQQRTHIFFSAKGDRAVCITSELVSQPTITWSVGSDRLRNDGFSQTWYGIQPGVCGILRSEVRIHRTTWRFGSTSKDYLCEVSASDSKTSDYKVLPNAHSTSNFALVAATTLTVTILCLLCCLYCMLTRPRASVY

SEQ ID NO: 229 - protein, 452 aa, Equid alphaherpesvirus 1
MPAVLLVLYVNPPPSVCILTQKLSLGLYNQWWRVCRSVPPPWYVFFNKRSMSTFKLMMDGRLVFAMAIAILSVVLSCGTCEKAKRAVRGRQDRPKEFPPPRYNYTILTRYNATALASPFINDQVKNVDLRIVTATRPCEMIALIAKTNIDSILKELAAAQKTYSARLTWFKIMPTCATPIHDVSYMKCNPKLSFAMCDERSDILWQASLITMAAETDDELGLVLAAPAHSASGLYRRVIEIDGRRIYTDFSVTIPSERCPIAFEQNFGNPDRCKTPEQYSRGEVFTRRFLGEFNFPQGEHMTWLKFWFVYDGGNLPVQFYEAQAFARPVPPDNHPGFDSVESEITQNKTDPKPGQADPKPNQPFKWPSIKHLAPRLDEVDEVIEPVTKPPKTSKSNSTFVGISVGLGIAGLVLVGVILYVCLRRKKELKKSAQNGLTRLRSTFKDVKYTQLP

SEQ ID NO: 230 - protein, 424 aa, Equid alphaherpesvirus 1
MAKLTGMFSAAILLSMAICSTAIIYRGEHMSMYLNASSEFAVYPTDQSLVLVGHLLFLDGQRLPTTNYSGLIELIHYNYSSVCYTVIQTISYESCPRVANNAFRSCLHKTSKHYHDYFRVNASVETNVLLNITKPQPTDSGAYILRVKLDHAPTADVFGVSAFVYDLKSKTVPDPMPTTQTVEPTTSYVSTPTYDYTDDVTTETESTSTSTQQAMTSTQTPSATWGTQLTTELPTNETVVIGQEALLCHWFQPSTRVPTLYLHLLGRTGNLPEDVLLVEDSEFLRTTSPAHRPSASPADGDDFKQTNSTSLKARNKIVAMVVIPTACVLMLLLVVVGAIINGAVRKHLLSCASRRIYRSGQGGASAAERRRLTCGPTLAASSESLADDTTSSPPTPKPSKKTKLETDPLMEQLNRKLEAIKEES

SEQ ID NO: 231 - protein, 550 aa, Equid alphaherpesvirus 1
MELLAASRACIFFGLVTVLDAWGVQQVELSEGAWAMIDGRDVLTPTNTTTRVTKAWTFLETPPGCAGDISVKKVCVSHSLCEDNIIIGKHCNLLTGEHGIALAEFNVVNGSLRRTDDVYFVNGTVFPILAETRSVLQIHRATPSIAGVYTLHVSIDGMMKHSVVLLTVKKPPKQPQPRLRVKTPPPVTVPQVPVKTHTDFVVHGYHSRVYADGESFELSVNLESHIVEPSFSAEIQWYYMNTSSSSCDLFRVFETCIFHPTAMACLHPEQHTCSFTSPIRATKILHRVYGNCSDHGNSWPSRCHSTLLGNRLYFIQPAQNRVDLLFKDTPASATGLYVFVLLYNGHPEAWTYTLLSTANHFMNVLTDVTRPRLGEHFYTDLGHKIITPHPSVATTEELGAWTRHYLAFLLVIICTCAALLVALVVWGCILYIRSNRKPYEVLNPFETVYTSVPSNDPSDEVLVFERLASDSDDSFDSDSDEELEYPPPPKPAPQLPPYQFVDGGDAPSGRSGFKVWFRDTPEASPVPLHKPTLQGPDYSRVASKLKSILK

SEQ ID NO: 232 - protein, 130 aa, Equid alphaherpesvirus 1
MSSNSDNTECFGGVNYAEGMRKRKRNPVRNSTFQEYLDARNARYPRSGSTSDSDEDYTTRSKYESDVSEFKKMMDLETLPPPKAEPQAQKAEPDAAKEEPVSTTSYILNEWVAPMIGHFLAMCMYELLFK

SEQ ID NO: 233 - protein, 219 aa, Equid alphaherpesvirus 1
MEKAEAAAVVIPLSVSNPSYRGSGMSDQEVSEEQSAGDAWVSAAMAAAEAVAAAATSTGIDNTNDYTYTAASENGDPGFTLGDNTYGPNGAASGCPSPPSPEVVGLEMVVVSSLAPEIAAAVPADTIFASAAAPATRVDDGNAPLLGPGQAQDYDSESGCYYSESDNETASMFIRRVGRRQARRHRRRRVALTVAGVILVVVLCAISGIVGAFLARVFP

SEQ ID NO: 234 - protein, 272 aa, Equid alphaherpesvirus 1; identical to SEQ ID NO: 224

SEQ ID NO: 235 - protein, 236 aa, Equid alphaherpesvirus 1; identical to SEQ ID NO: 223

SEQ ID NO: 236 - protein, 293 aa, Equid alphaherpesvirus 1; identical to SEQ ID NO: 222

SEQ ID NO: 237 - protein, 1487 aa, Equid alphaherpesvirus 1; identical to SEQ ID NO: 221

SEQ ID NO: 238 - protein, 360 aa, African swine fever virus
MPSTLQALTKKVLATQPVLKDDYCILERCGLWWHEAPITIYHTCIDEQILIKTASFKHGLTLNVALMKAVQENNHGLIELFTEWGADISFGLVTVNMECTRTYAKVRCEEKALSENKILEIFYNVQYVKTSSNIILCHELLSDNPLFLNNAQLKLRIFGELDTLSINFTLDNISFNEMLTRYWYSMAILYKLTEAIQYFYQRYSHFKDWRLICGVAYNNVFDLHEIYNKEKTNIDIDEMMQLACMYDCNYTTIYYCFMLGADINRAMITSVMNFCEGNLFLCIDLGADAFEESMEIASQTNNWILINILLFKNYSPDSSLLSIKTTDPEKINALLDEEKYKSKNMLIYEESLFHIYGVNI

SEQ ID NO: 239 - protein, 362 aa, African swine fever virus
MSTPISLQALAKKILATQHISKEHYFILKYCGLWWHGAPIMLSTNEDNQLMIKSAIFKDGLELNLALMKTVQENNYDLIELFTEWGADINSSLVTVNTEHTWNFCRELGAKILNEMDIVQIFYKIHRIKTSSNIILCHKLLSNNPLFQNIEELKVIICCFLEKISINFILNEITFNEMLTRYWYSIAILCKLTEAIQYFYQRYSHFKDWRLICGLSFNNVSDLHEMYHIKKVDMNIDEMIYLACMRDSNFLTIYYCFVLGADINRAMVTSVKNFYMNNLFFCIDLGANAFEESLELAKQKNHDILVEILSFKDYYSSNVSLLSLKTTDPEKINALLKNYRSKNIMRYKKLCPEIIRWARFII

SEQ ID NO: 240 - protein, 189 aa, African swine fever virus
MRSSKKINNKKNMFNIKMTISTLLIALIILLIIILVVFLYYKKQQPPKKVCKVDKDCGSGEHCVRGSCSSLSCLDAVKMDKRNIKIDSKISSCEFTPNFYRFTDTAADEQQEFGKTRHPIKITPSPSESHSPQEVCEKYCSWGTDDCTGWEYVGDEKEGTCYVYNNPHHPVLKYGKDHIIALPRNHKHA

SEQ ID NO: 241 - protein, 81 aa, African swine fever virus
MDTSLKNNDGALEADNKNYQDYRAEPDKTNDVLDVTKYNSMVDCCHKNYSTFTSEWYINERKYNDVPEGPKKAVVHRCTIL

SEQ ID NO: 242 - protein, 52 aa, African swine fever virus
MDDTLPKQMTPTDTSSSKEEQAHCNNKTLEKQPKNINDNKCTDSQNTESSKV

SEQ ID NO: 243 - protein, 356 aa, African swine fever virus
MQPSTLQALAKRALATQHVSKDDYYILERYGLWWHEAPISMYIDDDNQIMIRTLCFKEGIRLNTALVLAVKENNDDLIMLFTEWGANINYGLLFINNEHTRNLCRKLGAKEELETSEILRFFFETKCKITSSNVILCHELFSNNPFLQNVNMVDLRMIIYWELKDLTTNSMLNEISFSEMLTKYWYGIAVKYNLKEAIQYFCQEYRHFDEWRLICALSFNNVFDLHEICNTTKIHMSINKMMELACMRDNNFLTIYYCFALGANANRAMLISVKNFCIENMFFCMDLGANVIEHSKTLADIYGYSIIVNILSLKIYKANPILLSKETNPEKINTLLKNYYSKNMLAYDICCIDNYL

SEQ ID NO: 244 - protein, 42 aa, African swine fever virus
VANALLASACKVDGCMFILLKKIKLSGKHKDQPVVNICSSIF

SEQ ID NO: 245 - protein, 214 aa, African swine fever virus
MLGLQIFTLLSIPTLLYTYELELLDLTRTPPEKELEYWCTYANHCRFCWDCQDGICKNKVFENHSPILENDYIANCSVFRHNEFCTYYVTSIKPHEVYRTECPQHNHEWHEAVIRKWQKLLTYGFYLVGCVLVANYIRKRSLQTIIYLMVLLVIFFLLSQLMLYRELEAKKHKIGSIPPERELEHWCTHGKYCDFCWDCQNGICRNKVFKMIPP

SEQ ID NO: 246 - protein, 37 aa, African swine fever virus
MATCEQVAARQQIAVYQQIAVYQQQLIISKCCLWVSQ

SEQ ID NO: 247 - protein, 104 aa, African swine fever virus
MRFFSYLGLLLAGLASLQGFSTDNPLEEELRYWCQYVKNCRFCWACQDGLCKNKVLKDMPPVQEHSYPMEHCMIHRQCKYIRDGPIFQAECTMQTCDAIHLLNA

SEQ ID NO: 248 - protein, 124 aa, African swine fever virus
MLVIFLEILGLLANQVLGLPTQAGGHLRSTDNPPQEELGYWCTYMENCKFCWECAHGICKNKVNKSMPLIVENSYLTSCEVSRWYNQCTYGEGNGHYHVMDCSDPVPHNRPHQLLMKIYEKDDL

SEQ ID NO: 249 - protein, 51 aa, African swine fever virus
MYMVPHIKKNKRNYLILAVIFFQLATSQVTKELHCSFILRKRLNFGQKNHI

SEQ ID NO: 250 - protein, 124 aa, African swine fever virus
MLVVFLGILGLLANQVVGLPTQAGGHLRSTDNPPQEELGYWCTYMERCKFCWECAHGICKNKVNNSMPLIIENSYLTTCEVSRWYNQCTYGEGNGHYHVMDCSDPVPHNRPYQLLMKIYEKKDL

SEQ ID NO: 251 - protein, 205 aa, African swine fever virus
MLVIFLGILGLLASQVSSQLVGQLRPTEEPPEEELEYWCAYMESCQFCWDCQDGTCINKIDGSVIYKNEYVKSCLVSRWLDKCMYDLDKGIYHTMNCNQVLGLPNQPAGQLHPTDNPPQEELEYWCTYTENCKFCWNCQNGLCEGKLENTTILENEYVQSCIVSRWLNKCMYDLGQGIHHVMACSEPKPWNPYKILKREWKENNS

SEQ ID NO: 252 - protein, 137 aa, African swine fever virus
MLVIILGIIGLLASSNLVSSSTSTRVGGHLPLTFEPPENELGYWCTYVESCRFCWDCEDGICTSRVWGNNSTSIIENDYVKYCEVSRWGDLCRYDVEEHIYHSMNCSDPKPWNPYKIARKEWKKNEHPRKDLKKDEF

SEQ ID NO: 253 - protein, 127 aa, African swine fever virus
VATIKVLILVLLGVVVLQAAPIRKLEDLLPTRYPPEHELVYWCTYANQCDFCWECVHGICRNRIQTDWPVIHQNDWIINCTVSRWNGICSYYEGPKNHTDHQMDCANPTSHTYPHREYMKIYERDDL

SEQ ID NO: 254 - protein, 124 aa, African swine fever virus
MVRLFRNPIKCLFYRRSRKIQEKKLRKSLKKLNFYHPPEDCCQIYRLLENVPGGTYFITENMTNDLIMVVKDSVDKKIKSIKLYLHGSYIKIHQHYYINIYMYLMRYTQIYKYPLICFNKYYNI

SEQ ID NO: 255 - protein, 47 aa, African swine fever virus
MICNYKFVFFSYFATGNIKWWPVHLFNKKILTNLLLSLSLTYPVTIL

SEQ ID NO: 256 - protein, 290 aa, African swine fever virus
MKVIVFLLVLAVMQPVIQSQSFPGTGELPMTRRPPKRELEYWCTYAKSCDFCWNCRHGVCKNKVFEKHPLIKKNDYIQICRVSRYNERCSYFTDSRIRRFHIMSCTNPTYYDWFDELMQIKEDRVIDTENIKHTCLCMIATIALISYVRKQYSRMRMQAATRLLIFLGFYVLLGILMTNIIMNLPLSTDNPMQMRRPPERDLKFWCTYAKHCDFCWTCKDGMCKNKVFSDHPIITQNDYIVNCTVSRWHDRCMYEAHFRIHYQHNMNCSQPKDLEWFIELKRHVINQDDL

SEQ ID NO: 257 - protein, 63 aa, African swine fever virus
MAATYKLQLMGCNTATYRLHLIDRDSKGMKTLPSIQNLSFNPDNVSVYEKKFFLLMYEFLYES

SEQ ID NO: 258 - protein, 119 aa, African swine fever virus
MKLLALLCILIWLSQPGLNRPLSIFYMKQNLPRTYTPPIRELEYWCTYGKHCDFCWECRNGICKNKVWDDMPLIKQNDYISQCSIARYFDRCMYFIKPKSPYIHYMDCFQPTAYKGFSH

SEQ ID NO: 259 - protein, 116 aa, African swine fever virus
MKVLGLLLGYSVLILAHELPELPRTQHPPKEELPYWCTYVKNCDFCWDCQNGICKNKITNESNSMNSIVNCIVNRNSWGCFYEISVKMPNHHNMECSHPRPYTGNEIFMEKWGGGG

SEQ ID NO: 260 - protein, 119 aa, African swine fever virus
MKVFLGLLLGYSTILILTYQSPTTPWCFYEISLKIPNHHSMKCCSYPRLYEHEMFMEKWRDKNWPIIIRYYCFYLVFSFVFAGCVAFAICKNLRLSTTMKLLMLLSILVLLLSQPILNN

SEQ ID NO: 261 - protein, 274 aa, African swine fever virus
MKVLLGLLLGYSVLILAHELPDLPRTQHPPKSELSYWCTYVPQCDFCWDCQDGICKNKITESRFIDSNHSIVNCRVFRDSKTQSCLYEISSKMPNHFSMECLHPRPYTGNEIFMQTWGGGGGDHQQLSIKQYCLYFIIGIAYTGCFVCALCKNLRLSTTMKLFVLLSILVWLAQPVLNRPLSIFYTKQILPRTYTPPMRELEYWCTYGKHCDFCWDCKNGICKNKVLDDMPLIVQNDYISKCSITRFIDRCMYFIEPKIPYIHYMNCSLPTYFS

SEQ ID NO: 262 - protein, 387 aa, African swine fever virus
MNSLQVLTKKVLIETKAFSNYHEDDIFILQQLGLWWENGPIGFCKQCKMVTGGSMSCSDVDSYELDRALVKAVKENQTDLIKLFVLWGADINFGIMCAKTKQTKDLCIQLGANPEFLDVGLYNMFVYLVKRKKVLLAIEIYYDNILILDSFNSNDFHLLIDFVYNRFILYLDEKEEEMTRNTLVLKFWYKFAIDFNLTKPIHYLSKKFPHLDLWRLQTAIYLGNIDEVHHAYFQENIRLGLNVMMFLACARPGNKLGIYYCFALGADLDRALERLISFNSINREINRKIRGEKRLCIEGSYLSNVYFCIGLGANPYTKKIQEIIKQKHSNIMILLFSKKKILSPHSVLQNKILDPSDVHKMISTYKNTESFYPFSSLAVKLIQQANI

SEQ ID NO: 263 - protein, 42 aa, African swine fever virus
MFDLSSILIRGGGVLIALTLLWIIEYNEYFIDAKHFNSSSEA

SEQ ID NO: 264 - protein, 38 aa, African swine fever virus
MFIIHTNIMNFFCNIKLFLYHFRSIHKKNILKLFNFIE

SEQ ID NO: 265 - protein, 375 aa, African swine fever virus
MNSLQVLTKKVLIENKAFSNYHEDDIFILQQLGLWWENGPIGFCKQCKMVTSGSMSCSDVDSYELDRALVRAVKKNQTDLIKLFVLWGANINYGIICAKTERTKVLCIQLGADPKFLDVGLYNMFVDLIKQQKVLLAIDIYYDNISILDSFDSHDFYVLIDFIYNRFILNLDEKEKMIKNTYVLKFWFKIAIEFNLIKPIRFLSKKFPHLDYWRLKTAVYLGNVDEIHHAYFQENIRLDPNDMMSLACMYPQNKLGIYYCFALGANINTALETLIGFINHEVNREITFFSNYGIWSNVHFCISLGANPYTKKIQETLLRQEKNVMMKLLFKKGLLSPHSILHKKILEPSEVRKIISTYEYTETFHSFSSLRDNLR

SEQ ID NO: 266 - protein, 41 aa, African swine fever virus
MFNISSILIQGSVMILILLLWIINENFTDAGGNNFNHTVLM

SEQ ID NO: 267 - protein, 37 aa, African swine fever virus
MLKCLKIIVHQCCKFDQNLISHKKGTHQHCSFKFHDV

SEQ ID NO: 268 - protein, 44 aa, African swine fever virus
MFDSLLPTITGGGGGSLIALALLWFADYYVEFIEARLDSNITAV

SEQ ID NO: 269 - protein, 38 aa, African swine fever virus
MIKNRCIAQYSFGTSLEMIQATMITMYNSIVIFFFCNV

SEQ ID NO: 270 - protein, 69 aa, African swine fever virus
MLLYIVIIVACIISKLVPNEYWAIHLFFIIMIFMVYMYEKLDIHQKSQFWNYTMSGLSGHNVQVTCKCY

SEQ ID NO: 271 - protein, 268 aa, African swine fever virus
MVSLTTCCLKNIVNQHAYVENTVLLYHLGLRWNCKTLYQCTQCNGVNYTNSHSDQCKNKDLFLIKVIVKKNLAVARTLLSWGASPEYARLFCRNTEEEQALNVQHVADVPSSKILERLTMSYKGNDEQLLITFYLLNLSTNFSTNLREQVRFKIVSYIICDLAIHQTFKIFYAKNYSLSTLYCIFLAIYYKLYTALRKMVKIYPGLKSFAYLTGFMFDDETVMETYNSTDDEISECKNRIITIKGYYGNIHCRSDIDHMYAFSQNNFW

SEQ ID NO: 272 - protein, 330 aa, African swine fever virus
MQSLFNIALKALTLKNHVEFLKRDKEVLTRLGLCCKNYDLIHKCSECGNICPNGQQHGTCININYLLIYAVKRDNYMLAYRLLCWGANEKFAHYFRRPLPNLKPLLPKKELTPKDIKQLAYEHFHSDSELITVFEVFRKSRNINDCLEFFYKKNIEFEIYFARLYVYSKTFYRKSWYWFCIFMAVKHSMEHALKKITKTYIPTFYNKTTLPLVLFLSACFYENVEWMKNFFYKANKKIQQKMLSYGMEWAATHGKVRTFVCCYTLGGTASLKMYQKAYQNERYMIMALCSYLGNIQINNPWDSLNPYMMVQNKEKFLPLKFSEETQYFYI

SEQ ID NO: 273 - protein, 319 aa, African swine fever virus
MLSLQTLAKKAVAKQSVPEEYHYILKYCGLWWQNKPISLCHYCNYVILSSTPFKGELLHLDVALIMAIKENNYDVIRLFTEWGANIYYGLTCARTEQTQELCRKLGAKDSLNNKEIFTGLMRHKTSNNIILCHEIFDKNPMLEALNVQEMGEEIHRELKFFIFYILDNVPMNVFVKYCYAIAVKYKLKRAISFFYQTYGHLSMWRLMCAIYFNNVFDLHEIYEQKIVHMDIDKMMQLACMKDYNFLTIYYCFVLGGDIDQAITATQWHHQTNNLYFCKDLKDLKQNTLTARPLLLPNITDPKKIYTMLKNYLPTSSNSL

SEQ ID NO: 274 - protein, 350 aa, African swine fever virus
MVLSLQTLTKKVLASQYPAKCHPHFLKCCGLWWHNGPIMYHQKKIWTPYFKNGTNLNAALVKAVEENNHDLIELFTEWGANINYGLLSVNTEHTRDLCRQLGAKEQLNDQEILRFFYTLKRDLTSSNIIFCHEVFSNNPILDTINRFEVKGMIYEQLEGLMVETDILSEMFTKYWYAMAIECNLKEAICYFYQRYAHLHRWRLMCALFYNNVFELHEVYEKERIRIDMNEMLKWACRKNYNYLTIYYCCVVLGADINQAMFHSIQFYNIGNIFFCIDLGANAFEEGKTLAHQKDNSFIASMLSLNCYNMNDSLSLKETDPEVIKRMLKDYHSKNLSIAHKHYINDGFNDI

SEQ ID NO: 275 - protein, 345 aa, African swine fever virus
MFPSLQSFAKKVLARQQVSIEYHVILERCGLWWYKAPISLDCKHMLIKLPSFADGLDLNTALMLATKENNYQLIKLFTEWGADINYGLICANTPPVREFCWELGAKYRVDKKKIMHMFFKLIHPGTTSSNIILCLKLFNDNPFPAYVIIREIRSSIYWKLKRLVEDTDILSNMSDGDMLTIYCFIVALQDNLREAISYVYQHFKYLNTWWLICALCFNKLFDLHNLYEKEKIRMDVDEMMRMACTKDNNFLTIYYCFLLGANINSAMLACVRFYNMDNLFFCIDLGADAFEEAKALAEQRNYYLISHHLSLDIYSADSSLLTLKEADPNKIYHLLKNYKLKSMLA

SEQ ID NO: 276 - protein, 353 aa, African swine fever virus
MLPSLQSLTKKVLAGQCIPVDQYHVLKCCGLWWHNGPIMLHIRRNKLFIRSTCFSQGIELNIGLMKAVKENNHDLIKLFTEWGADINYGMICALTENTRDLCKELGAKEYLEREYILKIFFDTTRDKTSSNIIFCHEVFSNNPNLRIIDNLDLRGEIMWELRGLMEITFMLDHDDSFSTVLTKYWYAIAVDYDLKDAIRYFYQKYPRLHRWRLMCALFYNNVFDLHELYEIERVRMDIDEMMHIACIQDYSYSAIYYCFIMGANINQAMLVSIQNYNLGNLFFCIDLGADAFEEGKALAEQKENYLIAHALSLKHYNPVISLLSIVTDPEKINCMLKNYHSINMGIFLDYEQR

SEQ ID NO: 277 - protein, 531 aa, African swine fever virus
MFSLQNLCRKTLPNRKLPEFFDEYILQLLGLYWENHGTIQRAGNNCVLIQQHTLIPVNEALRTAASEENYEIVSLLLAWEGNLYYAIIGALEGNRHDLIRKYDDQIKDHHEILPFIDDPVIFHKCHIMRQCFFDCILYQAVKYSKFRVLLYFKHRLEDDLPFTHLLIEKACKDHNYEVIKWIYENLHIYNMIDTFECAIAHKDLHLYCLGYRFIYNRIVPDKYHHLDIRMLSSLQLLHKVAAKGYLDFILETLKYDHNKDNINIILTQAATYNHRKILIYFIPQSTHAQIEQCLLVAIKAKSSRKTLNLLLSHLNLSINLIKKISHYVATYNSTNIIGILSMRRKKKIYLDIILTKFVKKAIFNKFVVRCMDTFSINPERILKIAARINRMMLVKKISEHVWKNHAVRLKYLKHAVHTMKHKDGKNRLMNFIYDRCYYHMQGEEIFSLARFYAIHHAPKLFDVFYDCCILDTIRFKSLLLDCSHIIGKNAHDATNINIVNKYIGNLFVMGVLSKKEILQDYPSIYSKQYMP

SEQ ID NO: 278 - protein, 350 aa, African swine fever virus
MLPSLQSLTKKVLAGQCVPTNQHYLLKCYDLWWHDAPITFDHNLRLIKSAGIKEGLNLNTALVKAVRENNYNLIKLFAEWGADINYGLVSVNTEHTWDLCRELGAKETLNEEEILQIFIDLKFHKTSSNIILCHEVFSNNPILQKVNNIKMRIEIFWELRELIVKTDLLNNEFSLSTLLLKYWYAIAIRYNLKEAIQYFYQKYTHLNTWRLTCALCFNNVFDLHEAYEKDKIHMDIEEMMRIACIKDHNLSTMYYCYVLGANINQAMLSSIQYYNIENMFFCIDLGADVFEEGTTALGEGYELIKNILSLKIYSPATTPLPKSTDPEIIDHALKNYVSKNMMIFLTYDLR

SEQ ID NO: 279 - protein, 353 aa, African swine fever virus
MSLPLSLQTLVKKTIASQCLSIDEHCILKYCGLWWHDAPLKLCMDRGRIQIKSGFLGEDIDLRVALIIAVKENNYSLIKLFTEWGANINYGLLSINTKHIRELCRQLGAKETLEDNDIFRIFTRIMHNKTSGSIILCHEIFMNNPILENKFVIQLRGLIYKRLWGLIEIKETDELNGLLVKYWYAKAVQYDCKDAICFLDEKYTDLNEWRLKCLLYYNKIYELHEMYHKENIQIDVHDMICLASTKDNNPLTIYYCYALGGNINQAMLTSVQYYNIGNIFFCIDLGGNAFEEGRAIAEQKGYNFLSHSLALDIYSSDASLPLNLKDPEEISSLLKDYKSKNLSIIWEYSHNIL

SEQ ID NO: 280 - protein, 357 aa, African swine fever virus
MLSLQTLAKKVVACNYLSSDYDYTLQRFGLWWDLGPIHLCNNCKQVFSYKHLQCFSEDDLCLEAALVKAVKSDNLELIRLFVDWGANPEYGLIRVPAVYLKRLCAELGGLTPVSEPRLLEILKEVARLKSCAGVLLGYDMFCHNPLLETVTRTTLDTVTYTCSNIPLTGDTAHHLLTKFWFALALRHNFTKAIHYFYKRHKNHLYWRVACSLYFNNIFDIHELCREKEICISPNLMMKFACLREKNYAAIYYCHRLGASLDYGMNLSIYNNNTLNMFFCIDLGAADFDRAQLIAHKAYMYNLSNIFLVKQLFSRDVTLVLDVTEPQEIYDMLKTYTSKNLKRAEEYLTAHPEIIVID

SEQ ID NO: 281 - protein, 526 aa, African swine fever virus
MFSLQDLCRKHLFILPDVFGEHVLQRLGLYWRCHGSLQRIGDDHILIRRDLILSTNEALRMAGEEGNNEVVKLLLLWKGNLHYAVIGALQGDQYDLIHKYENQIGDFHFILPLIQDANTFEKCHALERFCGVSCLLKHATKYNMLPILQKYQEELSMRAYLHETLFELACLWQRYDVLKWIEQTIHVYDLKIMFNIAISKRDLTMYSLGYIFLFDRGNTEATLLTQHLKKTAAKGLLHFVLETLKYGGNIDTVLTQAVKYNHRKLLDYFLRQLPRKHIEKLLLLAVQEKASKKTLNLLLSHLNYSVKRIKKLPRYVIEYESTLVIKILLKKRVNLIDAMLEKMVRYFSATKVRTIMDELSISPERVIKMAIQKMRTDIVIHTSYVWEDDLERLTRLKNMVYTIKYEHGKKMLIKVMHGIYKNLLYGEREKVMFYLAKLYVAQNAATQFRDICKDCYKLDVARFKPRFKQLILDCLEIITKKSCYSILEILEKHIISLFTMKVMTEEEKNLCLEILYKVIHYKTIQC

SEQ ID NO: 282 - protein, 280 aa, African swine fever virus
MSSSLQELCRKKLPDCILPEFFDDYVLQLLGLHWQDHGSLQRIEKNQILVQQEPIHINEALKVAASEGNYEIVELLLSWEADPRYAVVGALESKYYDLVYKYYDQVKDCHDILPLIQNPETFERCHELNSTCSLKCLFKHAVINDMLPILQKYTDYLDRWEYCSQMLFELACSKKKYEMVVWIEGVLGVGKVTSLFTIAISNRDLQLYSLGYSIILENLYSCGQDPKFLLNHFLRDVSIKGLLPFVIKTIEYGGSKEIAITLAKKYQHKHILKYFETWES

SEQ ID NO: 283 - protein, 40 aa, African swine fever virus
MFPTDVLKRKHLFTKKNINFTGILMYDKTVVHWFEISKTI

SEQ ID NO: 284 - protein, 506 aa, African swine fever virus
MFSLQDICRKHLFQLPDAFDEYILQALGLYWEKHGSLQRIRKDAVFVQRNIVLSTNEALRIAASEGNERVIKLLLSWEGNFHYVIIGALEGDQYDLIHKYDSQIKDYHMILSLIQNANTFEKCHQLSNSNMWCLIQNAIKYNMLPILQKHRNILTHEGENQELFEMACEEQKYDIVLWIGQTLMLNEPEFIFDIAFERIDFSLLTMGYSLLFDNKMSSIDIHDEEDLTSLPTEHLEKAATKGCFFFMLETLKHGGNVNMAVLSKAVEYNHRKILDHFIRRQKCLSREEIENLLLTAITNCASIKTLNLLLSYLNYSVKNIIGKIVQHVIKDGDYTIILLLKKKKINLVEPVLTGFIDYYYSYCFIKHFIQEFAIRPEKLIKMAARKGKLNMIIEFLNEKYVHKDDLGTIFKYLKTLVCTMKHKKGKETLIVLIHKIYQDIHLETKEKFKLLRFYVMHDATIQFLSMCKDCFNLAGFKPFVLECLDIAIKKNYPDMIQYIEILSKSE

SEQ ID NO: 285 - protein, 498 aa, African swine fever virus
MFSLQEICRKNIYFLPDWLGEHVIQRLGLYWEKHGSLQRIGDNYVLIQQDLIIPINEALRMAGEEGNDEVVQLLLLWEGNIHYAIIGALESDHYSLIRKLYDQIEDCHDILPLIQDPKLFEKCHELDKSCNILCLVLHAVKNDMLCILQEYKMHLSGEDIQVVFETACRSQKNDIVSWMGQNIAIYNPEVIFDIAFDKMNVSLLSIGYTLLFNHHINNTNENINSLLTQHLEWAAGMGLLHFMLETLKYGGDVTIIVLSEAVKYDHRKILDYFLRRKNLYQEDLEELLLLAIRADCSKKTLNLLLSYLNYSINNIRKKILQCVKEYETTVIIKILRKRKINLIEPILADFIGYHSYTYMVDFMREFSIHPEKMIKMAARESREDLIIKFSKKVCKEPKDRLHYLKSLVYTMRHKEGKQLLIYTIHNLYKACHLESKEMFNLARFYARHNAVIQFKSICHDLSKLNINIKNLLLECLGIAIKKNYFQLIKTIETDMRYE

SEQ ID NO: 286 - protein, 525 aa, African swine fever virus
MFSLQDLCRKNIFFLPNDFSKHTLQWLGLYWKEHGSVHRAEKDSIMIQNELVLSINDALQLAGEEGDTDVVQLLLLWEGNLHYAIIGALKTEKYNLICEYHSQIQDWHILLPMIQDPETFEKCHDLSLGCDFICLLQHAVKYNMLSILVKYKEDLLNARIRHRIQSLFVLACENRRIEIIDWIGQNLPIPEPDAIFSIAVATRDLELFSLGYKIIFDYMQRQGIIQLTNGVRMVVLNRHISMAIDNGLLPFVLETLKHGGNIHRALSYAVTHNRRKILDYLIRQKNIAPNTIERLLYLAVKNQSSRKTLNLLLSYINYKVKNVKKLVEHVVNEKSTLVLKILLEKKENLVDAVLTRLVKHSTYFQVREFIQEFSISPEKFIKIAVREKKNVLIEAISEDIWENPTERITYLKQIVHTIKYESGRRFLVDIIHSIYQSYSLKHEDILKLATFYVKHNAITHFKDLCKYLWLNRGTESKKLFLECLEIADEKEFPDIKSIVSEYINYLFTAGAITKEEIMQAYDALE

SEQ ID NO: 287 - protein, 527 aa, African swine fever virus
MFSLQDLCRKNTFFLPSDFSKHTLHLLGLYWKGHGSIQRIKNDGVLIEHDLTLSINEALILAGEEGNNEVVKLLLLWEGNLHYAIIGALRTENYNLVCEYHSQIQDWHVLLPLIQDPETFEKCHDLSLECDLSCLLQHAVKYNMLSILVKYKEDLLNVLFRQQIQGLFILACENRKLEILTWMGQNLPIPDPEPIFSIAVVTKDLEMFSLGYKIVFEYMENQGLHLTQVVRMVMLNHHFGMVINKGLLPFVLEILNYGGNVNRALSYAVTQNKRKILDHVVRQKNIPHKTIERMLHLAVKKHAPRKTLNLLLSYINYKVKNVKKLLEHVVKYNSTLVIRLLLEKKKNLLDATLTRYVKDSTYFQVKEFMQDFSISPEKFIKIAVREKRNVLIKGISEDIWENPAERIRNLKQIVCTIKYESGRQFLINIIHTIYQSYSLKPEEILKLATFYVKHNATTHFKDLCKYLWLNRRTESKKLFLECLEIADKKEFPDIKSIVSEYINYLFTAGAITKEEIMQAYALEYAMY

SEQ ID NO: 288 - protein, 506 aa, African swine fever virus
MFSLQDLCRKNLFLPLEPLGKHVVQRLGLYWEGHGSVKRVGDCFICVDQIWMLSIHKAIQIAASEGNENIVKLFLLWKGSLQYAIIGALEGRQYDLIQKYYNQIGDCHQILPLIQDPEIYERCHELNVTCTFQCLFQHAIRDNMLPIFQKYGEDLNGNRRMVQLLYEMACRLQNYDIIKWIGSNLHVYNLEAIFSIAFVRKDLTLYSLGYMLLLGRMSTEDRNFISIITRHLEYASKKGLFDFVLESLKYGGQVDTVLFQAVKYNHRKILAHFIHEIPRETVEKLILHAVESRASRKTFNLLLSSINYCVNPFVKKLLHAVVKHKYMLIIKLLLERPKKKINLVDAALFKLVKYSTYTEIVKYMGEFSVDPKRVVKMAARLMRVDLIKKISNDAWEDKLERIKHLKQMVNTMNHRNGKNLLMYNIHNITGYTYLNTKEAFNLTRFYAVHNATCLFKEMCKSCFVHDKIQLRELLEDCLHIANRHDYIQIAETADECIKYIDLITFK

SEQ ID NO: 289 - protein, 542 aa, African swine fever virus
MFSLQELCRKNIYILPYPLAKHVLQQLGLYWKGHGSLQRIGDDHVLLQQDLIFSINEALRMAGEEGNNEVVKLLLLWEGNLHYAIIGALEGDRYDLIHKYYDQIGDCHKILPLIQDPQIFEKCHELSNSCNIRCLLEHAVKHDMLSILQKHKEQIRLHMALTQILFELACHERKNDIIRWIGYSLHIHHLETIFDVAFAHKNLSLYVLGYELLMHKVNTEAAYIELPNLLSYHLRTAAAGGLLNFMLETIKHGGYLDKTVLSAAIRYKHRKIVAHFIHQVPRKTVKKLLLYAVQARAPKKTLNLLLSSLNYSVHTITKQLVHNVVIYSSTLIVKLLLMRRKNKLNLVDAVLARLVKYSTYTDIVQFMGEFSVSPERVIKMAARESRTFLIEMISKAAWGNHPQTLIHHLKQLTNTMKPQSGKDHIIYTIHYIYLNSNMLVAEEEKNIFKLAKFYANHNAVNRFKQICEDYYILDARFKTLILECFEIAVQKNYPRIANIVDDYIRFLFYRGNITEEEIREAYSLKDAEVYVDLKWLQQGEMV

SEQ ID NO: 290 - protein, 224 aa, African swine fever virus
MFPKINTIDPYISLRLFEVKPKYVGYSSIDARNQSFAIHDIKNYEKFSNAGLFYTSPTEITCYCCGMKFCNWLYEKHPLQVHAFWSRNCGFMRATLGIIGLKKMIDSYNDYYNNEVFVKHQNRVYTHKRLEDMGFSKPFMRFILANAFIPPYRKYIHKIILNERYFTFKFAAHLLSFHKVNLDNQTTYCMTCGIEPIKKDENFCNACKTLNYKHYKTLNFSVKL

SEQ ID NO: 291 - protein, 50 aa, African swine fever virus
MSGFMNSLRKCIGNINSHLEGFMRTYLLRIIRKIKPTTQPSIEDDHYKCL

SEQ ID NO: 292 - protein, 104 aa, African swine fever virus
MSTKKKPTITKQELYSLVAADTQLNKALIERIFTSQQKIIQNALKHNQEVIIPPGIKFTVVTVKAKPARQGHNPATGEPIQIKAKPEHKAVKIRALKPVHDMLN

SEQ ID NO: 293 - protein, 236 aa, African swine fever virus
MRGILIAIEGINGVGKSTQAMKLKETLECMDYNAICIHFPNPDTTTGDLILQVLNKTIEMSSEQLHKLFTKHRSEFIAEIAVLLKLNYIVIVDRYIWSGLVYAQADGITIETKNTFKPDYTFFLSSKKPLNEKPLTLQRLFETKEKQETIFTNFTIIMDDVPKNRFCIIPATLNKEIIHMIILTKTLKVFDNNSCLNYIKMYDDKYLNVQDLNLFDFDWQKYIEDNNDKEEYDFIV

SEQ ID NO: 294 - protein, 60 aa, African swine fever virus
MHSNVSFNFIACVLFPTPLIPSMAMSIPRMINKRKKRIQFLTFLTNLFLYNIVQHCISGI

SEQ ID NO: 295 - protein, 158 aa, African swine fever virus
MNKKIIVMMALLHKEKLIECIYHELENGGTILLLTKNIVVSEISYIGNTYKYFTFNDNHDLISKEDLKGATSKNIAKMIYNWIIKNPQNNKIWSGEPRTQIYFENDLYHTNYNHKCIKDFWNVSTSVGPHIFNDRSIWCTKCTSFYPFTNIMSPNIFQ

SEQ ID NO: 296 - protein, 289 aa, African swine fever virus
MNKIFLNITETINMVLIEFLTGFFYLYGKRLFSISKVMDMICLDYYTIIPAPLAMMLAARLKNYDLMKRLHEWEISIDYALLVVDDVPSIDYCLSLGARSPTRAQKRELLRDNTFNPVYKYLMNCSGFPTKREKNIPCDVQCERLQKNIIKELVFNCSVLLEMVLHTEREYAYALHCAAKHNQLPILMYCWQQSTDAESILLKTCCSDKNINCFNYCILYGGAQNLDAAMVEAAKHDARMLINYCVMLGGRSLNEAKETAAMFGHIECAQHCFKLQSYVVDTSNTDDTD

SEQ ID NO: 297 - protein, 226 aa, African swine fever virus
MFPEREIENLFVKWIKKHIRNGNLTLFEEFFKTDPWIVNRCDKNGSSVFMWICIYGRIDFLKFLFEQESYPGEIINPHRRDKDGNSALHYLAEKKNHLILEGVLGYFGKNGTRICLPNFNGMTPVMKAAIRGRSLNMLSLIKFGADPTQKDYHRGFTAWDWAVFTGNMELVKSLNHDYQNLSTCISLFTSWMFSTGGLRKSPKLLLLAVNIMSMKNFLNRIQSSCV

SEQ ID NO: 298 - protein, 858 aa, African swine fever virus
MCAGFYVAVHPWLEAQSLHKVGHTGNLAARLHDGSYTTCFTDEWKYCFTLETSTKKDAQKIEAGVLYCAQFFRVKNKELVCLLPEKIKQLAEDVANCLDISYTLCDSPAYEMNDSTIVVEPSLPSDPLISKEKLRHLVITPVEDEHFADDVLFFSTDETRTAIEDRLYQKEAANMGYQELRRSGRAILQMACRCGKTRVAYLILSNYLQGKVLYLVPGLSLLRQTLEKLYQYGISLKNVLLVGSDQTRIVLNHDNIEMTTNPVFIAKRIREAPSLLVIATYQSSTLLVDDFDLIISDECHRICGEWETRPFTHVLLNFKKGHRLFLTATPRYDTPLSMKNRELFGGVAFRYYLREGIEAGYVNDFELQMVAAPKLAHQPSTKEETTKQIIVKQIIMALAYLKTNIPAPKMLVFTRDIKQAKELYAALVDQGVYALIAHSTLPRQVILKTFTEFCSSKEPVILLNCRLFQEGVEVPELNAVFFAAPRHSPRDIIQSICRPLNKQVQKPHATIFLPLEVNTENVCLDRFSSIIPFADALASEDPRFYEHLLNPSEVAYPINWIGAHGSVSELLQLARHAIRYGTQGKIDRLTRSERLPWKAAFAELKRTVEICCRYPKINDGFHFGGATLRFDTWYKWVIKSYLQYKNKEPSSLEPYQVSDLESLQDWTTRGVGGPYPWEESMAFLETWLAQNKGELVAIDIHQGGWIGLDATPMERLSGVLTTVSQRDGRSYGKNKKLRPKKGFMIPPQQAQDLDRIFGKHNLKWRKDRVNGFLKEDEHGNYTGEPTCIQEAYRTFKEYVKTNPEYIEKYWPGYAKGKHKHQELPHIWEKGLAPPRYKAFKDGNKQLIQRSPKKKDIKN

SEQ ID NO: 299 - protein, 179 aa, African swine fever virus
MEGEELIYHNIINEILVGYIKYYINDISEHELSPYQQQIKKILTYYDECLNKQVTITFSLTSVQEIKTQFTGVVTELFKDLINWGRICGFIVFSAKMAKYCKDANNHLESTVITTAYNFMKHNLLPWMISHGGQEEFLAFSLHSDMYSVIFNIKYFLSKFCNHMFFRSCVQLLRNCNLI

SEQ ID NO: 300 - protein, 137 aa, African swine fever virus
MEAVLTKLDQEEKKALQNFHRCAWEETKNIINDFLEIPEERCTYKFNSYTKKMELLFTPEFHTAWHEVPECREFILNFLRLISGHRVVLKGPTFVFTKEIKNLGIPSTINVDFQANIENMDDLQKGNLIGKMNIKEG

SEQ ID NO: 301 - protein, 317 aa, African swine fever virus
MVETQMDKLGFLLNHIGKQVTTKVLSNAHITQTMKEIILENHSVDGGAAKNVSKGKSSPKEKKHWTEFESWEQLSKSKRSFKEYWAERNEIVNTLLLNWDNVRGAIKKFLDDDREWCGRINMINGVPEIVEIIPSPYRAGENIYFGSEAMMPADIYSRVANKPAMFVFHTHPNLGSCCGGMPSICDISTTLRYLLMGWTAGHLIISSNQVGMLTVDKRIIVDLWANENPRWLMAQKILDIFMMLTSRRSLVNPWTLRDLKKILQDYGIEYIIFPSNDFFIYEDERLLMFSKKWTNFFTLHELLDDLETIETKASSTT

SEQ ID NO: 302 - protein, 334 aa, African swine fever virus
MLIFISNMEELLIENSQRFTIFPIQHPECWNWYKKLESLTWTAQEVDMCKDIDDWEAMPKPQREFYKQILAFFVVADEIVIENLLTNFMREIKVKEVLYFYTMQAAQECVHSEAYSIQVKTLIPDEKEQQRIFSGIEKHPIIKKMAQWVRQWMDPDRNTLGERLVGFAAVEGILFQNHFVAIQFLKEQNIMPGLVSYNEFISRDEGMHCSFACFLISNYVYNIPEEKIIHKILKEAVELVDEFINYAFDKARGRVPGFSKEMLFQYIRYFTDNLCFMMQCKSIYKVGNPFPQMTKFFLNEVEKTNFFELRPTQYQNCVKDDAFAFKLFLNDDDF

SEQ ID NO: 303 - protein, 778 aa, African swine fever virus
METFFIETLASDVYGKALNVDLDRLSQAQVKYTLQELISYCSALTILHYDYSTLAARLSVYQLHQSTASSFSKAVRLQAAQSCSRLSPQFVDVVYKYKAIFDSYIDYSRDYKLSLLGIETMKNSYLLKNKDGVIMERPQDAYMRVAIMIYGMGRVVNMKMILLTYDLLSQHVITHASPTMFNAGTKKPQLSSCFLLNVNDNLENLYDMVKTAGIISGGGGGIGLCLSGIRAKNSFISGSGLKSNGIQNYIVLQNASQCYANQGGLRPGAYAVYLELWHQDIFTFLQMPRLKGQMAEQRLNAPNLKYGLWVPDLFMEILEDQIHNRGDGKWYLFSPDQAPNLHKVFDLERSQHENAHREFKKLYYQYVAEKRYTGVTTAKEIIKEWFKTVVQVGNPYIGFKDAINRKSNLSHVGTITNSNLCIEVTIPCWEGDKAEQGVCNLAAVNLAAFIRENGYDYRGLIEASGNVTENLDNIIDNGYYPTEATRRSNMRHRPIGIGVFGLADVFASLKMKFGSPEAIAMDEAIHAALYYGAMRRSIELAKEKGSHPSFPGSAASKGLLQPDLWVRCGDLVSSWEERVAQTTQGVLTPKRWSQLRLAAMQGLRNGYVTALMPTATSSNSTGKNECFEPFTSNLYTRRTLSGEFIVLNKYLIDDLKEINLWTEAIQQQLLNAGGSIQHILDIPAEIRDRYKTSREMNQKILTKHAAARNPFVSQSMSLNYYFYEPELSQVLTVLVLGWKKGLTTGSYYCHFSPGAGTQKKIIRNSEKACNADCEACLL

SEQ ID NO: 304 - protein, 165 aa, African swine fever virus
MVNPNKRIMNKKSKQASISSILNFFFFYIMEYFVAVDNETSLGVFTSIEQCEETMKQYPGLHYVVFKYMCPADAENTDVVYLIPSLTLHTPMFVDHCPNRTKQARHVLKKINLVFEEESIENWKVSVNTVFPHVHNRLSAPKLSIDEANEAVEKFLIQAGRLMSL

SEQ ID NO: 305 - protein, 1048 aa, African swine fever virus
MQETFKFLRCNSQGEAVEDKYSLETLKNHFVVRDEYNNLFRVFSSRDDFWEWEAAQPFEQKCFHEVVFGFLPQRLKFDIDFPVNKSYSDDNVDDNDNVEDNVYDILDMIINVIMDVFYETYSLPYNINLTREQILLTDSIGLNKKRELKYSFHIILYTYSVLNNNEAKVFTSKVLENLPKHVYPFVDPQVNKSIQNFRIIGSHKKGSMRVKMFNEELAEVFETSTTTKKSDTLIATPFETTCLPCIFTNVKETTPSSCDTIQQSELEEVLKFAGTLCKNHCFLRVHKNLVLFKRTSPSYCEICKRMHDKDNTLILRVTGNKVYQHCRHDNKHSLLMGSLSGTNNFVETYVDQVMTKSIEVHESILFEELPDTQKHIYDESSMREYERVPTLVVKAQMKIGKTVQLRNYLQKYYGNNSISKQQTIRFVTFRQIFSKNIQSRLPNFTLYSEVTGDLDSYERVIIQVESLFRLTSTAEPVDLLILDEVESIFNQFNSGLHKYFAPSFAIFMWMLETANYVICLDANLGNRTYNILQRFRGDVPIFFHWNQYKRAQHDTYYFTSSRETWLNNLLKDLLEDKKIVIPTNSLMEARLLQSFIQKKFPEKKIGFYSSKSTAHERESHFNNVSYYWGLVDILIYTPTISAGVSYEDKRFDVLYGFFNNMSCDVETCCQMLGRVRELKSKCYKICLQGKQNYYPETIEDIEMFTLQKRDTLFQTISNHQLSFTYSKETGRPIYYKTPYYHLWLETMRIQHLSKNHFITRFINQIADTGAKVFILTGEKLETVKQYTSIKMEIKHQDYVNIASAETIDANKALQIKQNLKEGITVDQQDLFAYEKYKLLEFYAWHGHKITPKFVEQYNSFMTKQNYTGRVQISRGKTVYESLTMLQTQELNFHQWAMQHAEHHDLQFNYSFQSHMYAIMLLTKCGFKCVQDPNILTNEQLMTKLVDEFVQYDLSAISFEFKLKKPNKTDPQTILKFINKVLGLRYGLKIHHNKGNYYIKNTKAGSLIPFVRQQIKQSPCVVSNLLPITETSSAKEETSPIKEETFTET

SEQ ID NO: 306 - protein, 205 aa, African swine fever virus
MVEPREQFFQDLLSAVDQQMDTVKNDIKDIMKEKTSFMVSFENFIERYDTMEKNIQDLQNKYEEMAANLMTVMTDTKIQLGAIIAQLEILMINGTPLPAKKTTIKEAMPLPSSNTNNEQTSPPASGKTSETPKKNPTNAMFFTRSEWASSNTFREKFLTPEIQAILDEQFANKTGIERLHAEGLYMWRTQFSDEQKKMVKEMMKK

SEQ ID NO: 307 - protein, 78 aa, African swine fever virus
MPTKAGTKSTANKKTTKGSSKSGSSRGHTGKTHASSSMHSGMLYKDMVNIARSRGIPIYQNGSRLTKSELEKKIKRSK

SEQ ID NO: 308 - protein, 196 aa, African swine fever virus
MNIIRKLKPGTISLVLGPMFAGKTTFLIHCIYMLERLEKKVVFIKSTKNTRDKTIKTHSGIQLRPKQCKIIESTQLSDVGSLTDIHAVVVDEAHFFDDLITCRTWAEEEKIIILAGLNASFEQKMFPPIVRIFPYCSWVKYIGRTCMKCNQHNACFNVRKNADKTLILAGGSELYVTCCNNCLKNTFIKQLQPIKY

SEQ ID NO: 309 - protein, 145 aa, African swine fever virus
MDHYLKKLQDIYTKLEGHPFLFSPSKTNEKEFITLLNQALASTQLYRSIQQLFLTMYKLDPIGFINYIKTSKQEYLCLLINPKLVTKFLKITSFKIYINFRLKTFYISPNKYNNFYTAPSEEKTNHLLKEEKTWAKIVEEGGEES

SEQ ID NO: 310 - protein, 422 aa, African swine fever virus
MYTHVDVVGIAEASAALYVQKDRDRYLDVLTTIENFIYQHKCIITGESAHLLFLKKNIYLYEFYSNNVAEHSKALATLLYKLDPEYLTRYTVLITKIPNHWYVINVDQREFVRLYAIPAVKQHLPIPILPFYCTSALTQQELFCLGPELQLIQIYSKLCNPNFVEEWPTLLDYEKSMRMLFLEQFPQRLEMTGGKKEEKEKHESIIKKIILEMVSTRQRIVVGGYIQKNLYNHVLKNRNRLQLITSLNIYEEKDIIQQFCDSNGLKIKIRINNPLLPTNPELRRLTIYFNHNNDDDQSYLIVDMYNTGSYELVPTNQINTLDGSFLIGTPFVQARFLLVEIWVLMLIAQQTKKDTKKIIQFFINQYEMLMNSPWPSMEALFPSSSKRYLGNYVDPNALIKWAQLKLKRIPPFYPGKPDEESC

SEQ ID NO: 311 - protein, 1242 aa, African swine fever virus
MEPLRPQITYGPIETVDNEELTEADMLSFISAAVNSTGLIGYNIKSFDDLMDNGIPQIVKQMFNVDITYKDQRDHTEIDKLRESVQIQFNFTDVNIERPQHRNYSQGNKINLLPNKARLCGLSYSGPVNLAAEVILTAHYSNGRQEVKRASIPPFQVSTFPIMRGSNRCHTHHLSKTAKKEIGEDPNEPGGYFIARGGEWVVDLLENIRFNTLHIHYHTMQQGNNEIIRGEFISQPGGAFENSSQIIIRYMTTGAITIEINSTKFSKLRIPWYLIFRMFGMTGDDSIIEQVVFDLESNSLVNTFMIEILEKSIHVLDPIFQPVQHELNREKIIQFLSEKVSKFVSNPSAYKSDENAVQYLNERQLTILDKILLPHMGQTADTRVRKLRFLGLLIHKILLVIMNVFPPTDRDSYRTKRVHGSGVSLAKAFKAIFNTSVIAPIINGFKELLKQTAFEELTQRNIIEAFSAALSKNTASDLNRSMEQSIISGNKTIMVRQRPIVNRVSTQSLERKNLLNTISALRTVNTHNTTNASKQTERADMMRRVHASYPGYICVAQSADTGEKVGMSKQLAITANVCTAGEVLSLKQRLLSDPAIQQLADVSNKDIVRKGLARVFINGEWIGCCTNAFELAQRYRMLRREGKVVHPHTTIYWDSMVDEVEFWLDVGRLTRPLLIVDNNIEKYNQACYKAAEARKKGDKDWEKHKIPFIQNTRFTPQMAKDILAGTLTLEDLVAQGICEFITPEEAENCLVAFSIIELRKHKHDVTRRFTHVDVPQAILGLAALVSPYANCTQPARVTYETNQGRQTGGWYCFSWPYRVDMNRFFQFYNEMPLVKTIAHNYVIPNGLNTIVAYMIYGGYNQEDSVIVSQSFIDRGGFAGTFYREEKVELESDIESFGKPDPLITKNLKPGANYEKLVDGFVPVGTVVKKGDIIIGKVAKIRGEKDELNKYIDRSVMYGFDEPAVVDAVMRPHGPNDEIFGLMRLRYERNLNIGDKMSSRSGNKGIAALALPTSDMPFTEDGLQPDLIVNPHSHPSRMTNGQMIETTVGLANALQGVVTDGTAFLPINVQLLSERLAQEGLRFNGCQKMFNGQTGEYFDAAIFIGPTYHQRLQKFVLDDRYAVASYGPTDALTGQPLDGKRSHGGLRLGEMEHWVLTAQGAMQTIIEKSHDDSDGCISYICRNCGEPAIYNASHPIYKCMNCDVQADIGMVDSRRSSIVFQHEMRAANVNITSVLSPRVFQPA

SEQ ID NO: 312 - protein, 84 aa, African swine fever virus
MPYARDITKFITATEPEVGLPLLALQRSKSIIGVILLVISLLFIFIGIIILSVSSGHTTAASIFIVLSLILGGGGFFLIYKDNS

SEQ ID NO: 313 - protein, 418 aa, African swine fever virus
MSNYYYYYGGGRYDWLKTVEPTNFLKIGLPYQAHPLHLQHQATTPPSILEKFKRADILLNEVKAEMDPLMLQPETEKKLFQILSSIDMFKGLRKKVEFTYNAQIVTNAWLKMYELLNTMNFNNTSQAFCNCELPGGFISAINHFNYTMMHYPTFNWVASSLYPSSETDALEDHYGLYQCNPDNWLMQSPLLKKNIDYNNGDVTIASNVKNLALRATQRLTPIHLYTADGGINVGHDYNKQEELNLKLHFGQALTGLLSLSKGGNMILKHYTLNHAFTLSLICVFSHFFEELYITKPTSSRPTNSETYIVGKNRLRLFTPKEEQVLLKRLEFFNDTPLVDLSLYQNLLESVYFAVETIHLKQQIEFLNFGMKCYRHFYNKIKLLNDYLAPKKKIFQDRWRVLNKLYVLEKKHKLKLCAS

SEQ ID NO: 314 - protein, 152 aa, African swine fever virus
MYSILIACLVLLLCLVIYVGHRADHARKYLEGMWHGDPVFLKQSGLQSFYLYIQPDHTCFFSIVNKNGEKLMETKIPCTITNKIYMFFKPIFEFHVVMEDIHSYFPKQFNFLLDSTEGKLILENNHVIYAVLYKDNFATALGKTVEKYITQN

SEQ ID NO: 315 - protein, 158 aa, African swine fever virus
MFSNKKYIGLINKKEGLKKKIDDYSILIIGILIGTNILSLIINIIGEINKPICYQNDDKIFYCPKDWVGYNNVCYYFGNEEKNYNNASNYCKQLNSTLTNNNTILVNLTKTLNLTKTYNHESNYWVNYSLIKNESVLLRDSGYYKKQKHVSLLYICSK

SEQ ID NO: 316 - protein, 360 aa, African swine fever virus
MIILIFLIFSNIVLSIDYWVSFNKTIILDSNITNDNNDINGVSWNFFNNSFNTLATCGKAGNFCECSNYSTSIYNITNNCSLTIFPHNDVFDTTYQVVWNQIINYTIKLLTPATPPNITYNCTNFLITCKKNNGTNTNIYLNINDTFVKYTNESILEYNWNNSNINNFTATCIINNTISTSNETTLINCTYLTLSSNYFYTFFKLYYIPLSIIIGITISILLISIITFLSLRKRKKHVEEIESPPPESNEEEQCQHDDTTSIHEPSPREPLLPKPYSRYQYNTPIYYMRPSTQPLNPFPLPKPCPPPKPCPPPKPCPPPKPCPSAESYSPPKPLPSIPLLPNIPPLSTQNISLIHVDRII

SEQ ID NO: 317 - protein, 369 aa, African swine fever virus
MYFLVADHREHHVIPFLKTDFHHMHQNPIQKNQALLEIKQLFTGDYLICKSPSTILACIERKTYKDFAASLKDGRYKNRQKMLSLREQTNCQLYFFVEGPAFPNPQKKINHVAYASIITAMTHLMVRDHIFVIQTKNEAHSSQKLVQLFYAFSKEMVCVVPTSLTPTDEELCIKLWSSLSGISGVIGKILANTCSVAHLVHGKLSSQNIDQLKTPSNRPFPKKVKRMLISISKGNKELEIKLLSGVPNIGKKLAAEILKDHALLFFLNQPVECLANIQIVQKTRTIKLGMKRAEAIHYFLNWCGSAHVTDDSQNITEASRSTMQVATQSAAIQPAATQPLHEVSDDASSDASSPVGYQTLSKEMLLNTA

SEQ ID NO: 318 - protein, 1249 aa, African swine fever virus
MEEVITIAQIVHRGTDILSLNNEEIEALVDEIYSTLKGSNDIKNIRLIDFLFTLKDFVNHVRAEQSKLPDLSMPIEAYIRQLLVDPDVVPIVSEKKKELRVRPSTRKEIFLINGTHLAVPAEAPIEIYGLKLRLKTFSPQCFMRMAEIGSFSPETLGYVASGANLTNFIRVFMKCVDQETWKKNGEGVVVTTKENIIQFTHQYIELYKFLRSGGHSWLINRLAEEMVHRKLDREDQGSHISNIVETEEIEPEENIKRVIFFLKELSTMYSVSPVFTSGYMPLLYDLYRAGYLEVLWNPVEQKFLQHAEQREKEQMILQQVDMKLTEVITQARQYFKIMEEKIGRVQSDAIREILTMEGKVDDPNSILQEVIKACGKQEAELITTEYLNIKKQWELQEKNACAHLKLVKQLRSGLQYAELLKVLESIRVLYKEKNNTTNWNLCKACGFKLLCPHVDMLIQLQAAEASYDTMRTKLMKFSGINKEKENNQGLIYSYFCKICGEELAHFIQEDRTADVGIIGDLNSKLRVFIWQETMKACTFIHFGKLVDVKQFANIAVNVCLPLVYSIENIKKEEDYDPLTQLYAVIYIYAYILNLIYSSQKNKEFLTITIHGMKADSSLNAYVTFLLEKMMQQYSGIINQLSEITDQWIANNFREAFKKIIHQNGLQGLSVQDDTKVLLTEILLDPMYDYAATVARIDGSIPMHKPRTPKEAEYEFKTVIGRTPAELLSQKEFYDKIYTSKYRPDFTQLTRLNDIYFQEESLRVWWGGRDEEKTSTLIYLRAYELFLKYLQNAPNFNSELAEFKTYENAYGEQKALLAQQGFYNIFDPNTGRADQRTRLFEYKRLPISTLYDERGLPHKWTIYVYKAVDSSQKPAEIEVTRKDVIKKIDNHYALADLRCSVCHVLQHEVGQLNIKKVQTALKASLEFNTFYAFYESRCPKGGLHDFQDKKCVKCGLFTYIIYDHLSQPELVHDYYNNYKDQYDKEKMSIRSIQIKKDMTTPSTETQPKPPQEPWTFDYGKIIKTAKILDISPAVIEAIGAMEGRSYADIREGQGAPPPPTSMDDPRLMAVDSAVRIFLYNYNCLRHVSTFNKPPIHVERLVKHLSYEEKEDLEKVLPNVVNEYHTTFKHLRVTDPASALLYSIEFLCISFLTLYEIKEPSWVVNIVREFALTELNTIIQSEKLLSKPGAFNFMIFGEDFVCSGEDSSMDDISAYSSPGLFGEDIIDRLDDPFSIEDVDISLDVLDNLAPQ

SEQ ID NO: 319 - protein, 448 aa, African swine fever virus
MSNESFPETLENLLSMLQTKQQNAIQSEVIEWLHSFCETFHLKIHCHKQFIPSGEKKRAKIPAQETQGNTQPSHHVYRVVLSRAQPVKAQESLLTTMCNGLVLDANTWTCLAIPPPAPFQQATRQVQHFYRNNFYEVVPIQDGTLLTIYHWDDPEYGPSWCLASTHGYDVSNYCWIGDKTFAELVYELLQQHSTCDVTLEKNKTRGTRLFFDNLNPDYCYTIGIRHHNLQPLIYDPQNIWAIQSTNLKTLKTVYPEYYGYIGIPGIQSQVPELPQYDLPYLIRSYKTAMNQAKNAIKNGKKDKGYFNYGYLLISRAPAITKSTSNVLLKSPLLVFLQKSVYQKKHNISNSQRLEFIILQNYLMQHFRDHFIALFPQYISYYTKYQNMLNMIIHSIATKDKDHPFAGAVVKKVLEDIENAENIIDHTTIQNYAHQSKYAMLYLSIISHF

SEQ ID NO: 320 - protein, 129 aa, African swine fever virus
MEHPSTNYTPEQQHEKLKHYVLIPKHLWSYIKYGTHVRYYTTQNVFRVGGFVLQNPYEAVIKNEVKTAIRLQNSFNTKAKGHVTWAVPYDNISKLYAKPDAIMLTIQENVEKALHALNQNVLTLASKIR

SEQ ID NO: 321 - protein, 79 aa, African swine fever virus
MDQEQLFDKLYSLNLQLTAKNDQKKENRFFIRSGKKIQRIQMMMFITVYDINQKQKKRYGLRGCNLNLKATVLPLHKRI

SEQ ID NO: 322 - protein, 717 aa, African swine fever virus
MTKLAQWMFEQYVKDLNLKNRGSPSFRKWLTLQPSLLRYSGVMRANAFDILKYGYPMQQSGYTVATLEIHFKNIRSSFANIYWNRDSEEPEYVCCCATYQSHDGEYRYRFVWYQPFIEAYNAIEAALDPLETIILNLIAARDLDFVVHIFPYNKGHEDYLASTQLILKIFIATLLMDILRIKDNTLDVHLNSDYIIVMERLWPHIKDAIEHFFEAHKDLLGYLIAFRNGGNFAGSLRPSCGQKIVPLTIREVLQMNDINLAVWREVFIMQECSDLVINGIAPCFPIFNTWTYLQGINQIFFENTSLQEKFKKDFIARELSKEIIKGQKTLNDKEFKKLSLHQIQYMESFLLMSDVAIMITTEYVGYTLQSLPGIISRSSYLSPIVKNILMDEDSFMSLLFDLCYGAYVLHKKENVIHADLHLNNMTYYHFNPTSFTDRNKPGKYTLKVKNPVIAFITGPKVETETYVFKHIDGFGCIIDFSRAIMGPNHAIKLERQYGLAFVNTFYRNQSEHILKVLRYYFPEMLTNRENEIQGVILSNFNFFFNSITAIDFYAIARNLRSMLSLDYLHTSEVKRNVEISQTFLDTCQFLEEKAVEFLFKNLHTVLSGKPVEKTAGDVLLPIVFKKFLYPNIPKNILRSFTVIDVYNYNNIKRYSGKAIQTFPPWAQTKEILTHAEGRTFEDIFPRGELVFKKAYAENNHLDKILQRIREQLANENL

SEQ ID NO: 323 - protein, 105 aa, African swine fever virus
MKICKACSSCMVRTYVDGNIIFRCSCGESVQGDSQNLLVSSKVYHTGEMEDKYKIFIKNAPFDPTNCQIKKDCPNCHLDYLTQICIGSQKIIILVCRCGYMSNRG

SEQ ID NO: 324 - protein, 257 aa, African swine fever virus
MYSVCDVVRDAVAQSHLCACPNDKLPQCKGVTKAPPKCSVFHVAKLQDTKFKWKYTLDPLKAQKLSQIDKDIEKDAITLKLIYGIELSPEDLEWWKMQRCLINKKTGAKGGQFANKYLERQDLELLGYSPTPIIGGDFMFTALPDKVLRTIPVAWDRFLNPAMMIFFLIILLCVILGIFYVLVRNTLRRKQKSKQHQMEIKRFIKEKEQDPYIHTSFESWPADPNKEWKDLIPVYEAQGYCMADYRKKLGMPPGPNC

SEQ ID NO: 325 - protein, 475 aa, African swine fever virus
MTSLLKTDFNVSKYRLIAQKREANAVEIEAALEVVREFIIKKKLILYGGIAIDYALHLKGSSIYPEGERPDFDMFSPNHVEDAYELADILYEKGFKQVGTVRAIHVQTMRVRTDFVWVADLSYMPPNIFNTIPTLTYKNLKIIHPDYQRAGLHLAFCFPFDNPPREDVFSRFKKDLQRYNLIEKYYPIPVVPVKSTYESKTFSIPFKQVAIHGFAAYALLYQTLNELRITCKVPEWKTEFPQPSYSYHKNDKNITLTVDMPKAYPALVLATYNPEEVIKEMGLHLTEICEPYMDYSPPIFKTNDIHFFSTMFKELAISIIQDNLIVVSPQYLLLYFLYGAFATPADKSLFLFYYNATLWILEKADSLLNIIQKQTSPEEFTRFANTSPFVLTTRVLSCSQERCTFSPAYRISLANDVQQSQLPLPKTHFLSNSLPDVSTLPYNYYPGKGKDRPTNFSYEKNLLFNIGGKCTPSAM

SEQ ID NO: 326 - protein, 315 aa, African swine fever virus
MDALLKEIEKLSQPSLQKENNDVCDLCFMQMKKISNYQLLCEECGQLKDWFEPEYNEKFTVYSRLKIVGANSSYHQRDLDKANSSDYSSLQFHHILEELKSLNVKYMDAGQKPFPIQVLKETAHSYNQVQQHRVIRSITKLQILASILRSICLKLNIACTVADAARFTQLNTKGISRGMDLLRSLFVDNKITLNVDLNPIDSFINSTYSALQIKQIHQELQEENVYNLKEIVKSFILYADEKNIGVDLNRRTVVIATMYNVLRRAYYPIEIDTVVYQCKIRKNTITRALKMYEDYYSHFKSLYEQYHLNAAKKLI

SEQ ID NO: 327 - protein, 147 aa, African swine fever virus
MADNDNEDLIMDDLVEEYVETEEENLVDSEEESEDKDEIVESPSICEGFVQASSQTLVIIPDNERITSNVLTTFEATRLVAVRAQQLAINGSTMLKKKYSSPIDIAKQELFNRKIPLLVMRCIKVTPEGQKIVEIWNPREMGIPLLD

SEQ ID NO: 328 - protein, 62 aa, African swine fever virus
MNWGSISSGTPGLFVESIRNTPSVVKINVIFLKVISNTAVSVFWRDRRIRFESDWLNSYFQK

SEQ ID NO: 329 - protein, 962 aa, African swine fever virus
MREESWEDHDTIQLTAQRKYLAEVQALETLLTRELSVFLTEPGSKKTNIINRITGKTYALPSTELLRLYEHLEQCRKQGALMYFLERQGTYSGLMLDYDLKLNTNAVPPLEPPALSRLCHRIFVHIKNSSVLPEGSHKIHFFFTLKPEVVQGKYGFHVLIPGLKLAASTKKSIIGSLQHDATVQKILHEQGVTNPESCLDPHSASVPSLLYGSSKLNHKPYQLKTGFELVFDSSDPDYIPIHQIKNLESYNLVSELSLTNEQGSLVRPVYCAADIAAEKEEEIPTEDHSLSILMLHDPEARYLHKILNLLPPEYYVEYPLWSNVVFALANTSANYRPLAEWFSQKCPEKWNTGGKEKLEKLWNDASHHTEKKITKRSIMYWAHKHAPQQYKEIVEQGYFSILAEYVYSYNGMLEHYMIAKVIYAMMGNKFVVDVDSNGKYVWFEFVLPGQPMNQGEIWKWRKEVNPDELHIYISENFSRVMDRITEHIKYHLSQPHESNILNYYKKLLKAFERSKSKIFNDSFKKGVIRQAEFLFRQRSFIQTLDTNPHLLGVGNGVLSIETIPAKLINHFHEHPIHQYTHICYVPFNPENPWTKLLLNALQDIIPELDARLWIMFYLSTAIFRGLKEALMLLWLGGGCNGKTFLMRLVAMVLGDHYASKLNISLLTSCRETAEKPNSAFMRLKGRGYGYFEETNKSEVLNTSRLKEMVNPGDVTARELNQKQESFQMTATMVAASNYNFIIDTTDHGTWRRLRHYRSKVKFCHNPDPSNPYEKKEDPRFIHEYIMDPDCQNAFFSILVYFWEKLQKEYNGQIKKVFCPTIESETEAYRKSQDTLHRFITERVVESPSAETVYNLSEVVTAYAEWYNTNINVKRHIALELSQELENSVLEKYLQWSPNKTRILKGCRILHKFETLQPGESYIGVSTAGTLLNTPICEPKNKWWEWSPNPSAPPEKEASAPTP

SEQ ID NO: 330 - protein, 52 aa, African swine fever virus
MKKKDAYEQAKAYNILNVAFLLPVMALPKDMLLRIFPKELVQTLLSQEGQRD

SEQ ID NO: 331 - protein, 962 aa, African swine fever virus
MGKPTLLEPGHLYNVPAEHKNDVPIHYIITWIKQRLPEFGGAIPTSLADRVLIIKSRTGSGKSTALPVHVFRILRNENTHSFQKYLGRSVICTQPRVLTAVTLAKDIGASTHYPDMILGQTVGYQTKPLTEKPNRGLIYATAGVLLAQLHTMTDDEIASRYAFMIIDEAHERALGIDLMLMYIKSMLQRMLQRGSIGALRIPFVILTSATIDTHKYSTYFGIGKENIILVEGRQYGVETHWPLYNTNNYIKTACETALTIHKENIHDRPTEADILIFMPGMAEIRFLSMLLNNANMDLAKEKLPLMLILPIDSEAIAQENEAYLGLKAEIKNLWVKNPLTAKVEKPLRRVIVSTVVAETGLTIETLKYVIDPGWNRSIETYYPEWAGGLITRPAAQSRIEQRKGRVGRVFPGHFYPLYTKHVFEQIPVQQYPEIITEGPGAIFLSIVVETIKKNKEGVFKAEEIDMLDPPPTDALASAIERAIVAGLLTRGEKGLQLTQLGDIASRFSFLSIEEARMCFSGYFWQAAISDIATILAVVSVADKKLTNLLDSKQRNGAMLAEAVLAGIPPFLQNIDNAYTNIHLLLADDLLEGLFIFEGFQHAIVYFINNKVNNVAKHLREWCEKKMLKYSSMVQILARREDILNELAIVGLNPFHQWQNRLASANAETFLKRVCTLKQCMYEAYRLNCFCYDEHRLLYTGRNGIHFSYHDAVIKNPSCIVTPRIMLSPVSKQYMEWRLEPSFVSVLDGFVNVDINFLLPRQEIPNILGGVENEEEEPPLPIQVFLHKYVKTHFHFSGKSFKELKMKPSQMIKFPETTLINMIPDIPKNVVQTYLEINVCHRYSFKRLIYCETFYTDMDDVQHENSVELIGLPMAAHHLTINDFNKLYHLLKPDGFLIMYDLHQGQEAFWLHSLQDALGHHTIRRDMDFHTIPEWETIFKECGFTPIFSKQPSEHELFIVFKK

SEQ ID NO: 332 - protein, 119 aa, African swine fever virus
MLHWGPKYWRSLHLYAIFFSDAPSWKEKYEAIQWILNFIESLPCTRCQHHAFSYLTKNPLTLNNSEDFQYWTFAFHNNVNNRLNKKIISWSEYKNIYEQSILKTIEYGKTDFIGAWSSL

SEQ ID NO: 333 - protein, 318 aa, African swine fever virus
MLHLIYISIIVVLIIILISYTRKPKYFRITAPRSVALFHGIHPLNPKNYKTFSEEFETILNNAIEDGDFKGQLTEPCSYALRGGKYIRPIILMEIVRACQLQHSFGAPIYPAEAALAAEYFHVASLIIDDMPSFDNDVKRRNKDTVWARFGVAKAQMSALALTMQGFQNICRQIDWIKEHCPRFPDPNQLGALLCTFVSHSLNSAGSGQLVDTPEKTIPFFKIAFIMGWVLGTGSVEDIGMIERAAHCFGHAFQLADDIKDHDTDTGWNYAKIHGKQKTFDDVAQSLQECKKILHGKKIFTSIWNEIFQKVINVALGT

SEQ ID NO: 334 - protein, 438 aa, African swine fever virus
MYHDYASKLLADYRSDPPLWESDLPRHNRYSDNILNSRYCGNKNGAAPVYNEYTNSPEKAEKGLQLSDLRNFSFMLNPQHKNIGYGDAQDLEPYSSIPKNKLFNHFKNHRPAFSTHTENLIRRNVVRTEKKTFPQVASLKGTQKNCLTQPSSLPSLKNPKNSSVPSTRFSEHTKFFSYEDLPKLRTKGTIKHEQHLGDQMPGQHYNGYIPHKDVYNILCLAHNLPASVEKGIAGRGIPLGNPHVKPNIEQELIKSTSTYTDVPMLGPLPPKDSQHGREYQEFSANRHMLQVSNILHSVFANHSIKPQILEDIPVLNAQLTSIKPVSPFLNKAYQTHYMENIVTLVPRFKSIANYSSPIPNYSKRNSGQAEYFDTSKQTISRHNNYIPKYTGGIGDSKLDSTFPKDFNASSVPLTSAEKDHSLRGDNSACCISSISPSL

SEQ ID NO: 335 - protein, 164 aa, African swine fever virus
MNVDFIAGINNLGEKIYTCEPFKTSFQNPFIVALIITAVVLVVFFAICNPPVDKKRKTKTAIYVYICIVALLFLHYYVLNHQLNDIYNKSNMDVIVSSIHDKYKGGDEIIPPISPPSVSNELEEDQPKKIPAGPKPADSKPVSLPDSKPLVPLQEVIMPSQYNN

SEQ ID NO: 336 - protein, 475 aa, African swine fever virus
MDQEESHVISIFETLGAYFINIFYNFLYKNALYKKHSIVTEYQYQVKGYILGVKQNKKLYEKMLDSFYKYFCNITQINSKTLNFSNFVTTIVDSFIPKEYSQSISLEKKESILELLLCDYISNLGTFITTEKMLPFIIKNRKENYHKVTKEMQDYSLTFLLKKRMELYNKFLRKQAYVEPETELEETYARLSSYNRSLLHQIEELTSENKSLLADLSTLRKKYEKRQSEYRRLVQLLYQQIQRSSTSKSSYPLTKFIETLPSEHFSNEEYQKETPADQKEVVEMELLRKQELLTSQELTSKSPNNYPVPHSRTIVSKPLDNYPVPRSRTTTKIDFDNSLQNQELHTKNGFSEKDIVEFGQDKPEEENILAIDQDKPEEETILAIKQDISEEDNIFAIDQDKPEFNQDTPEFKEAVLDIKENILEEENQDEPIVQNPFLENFWKPEQKTFNQSGLFEESSNFSNDWSGGDVTLNFS

SEQ ID NO: 337 - protein, 354 aa, African swine fever virus
MALTTHSGKLIPELQFKAHHFIDKTTVLYGPSKTGKTVYVKHIMKILQPHIEQILVVAPSEPSNRSYEGFVHPTLIHYRLWLADKQKKNDNKGAERFLEAIWQRQTMMSSIYSRVNNIDMLKTLYHKLPIDIQQKENKNIAKVECLKAEQTDQKKEEKITSLYQQLLKKIIIQNIHMYKNLCLTEDEKFTLNYINLNPRLLLILDDCAAELHPLFTKEIFKKFFYQNRHCFISMIICCQDDTDLPANLRKNAFVSIFTNASICMSNFSRQSNRYSKQDKEYVEEISHIVFKGYRKLVYIREDEHRQHFYHSTVPLPTAFSFGSKALLKLCKAVYSKEVVIDKSNPYWSKFRLNF

SEQ ID NO: 338 - protein, 530 aa, African swine fever virus
MAEFNIDELLKNVLEDPSTEISEETLKQLYQRTNPYKQFKNDSRVAFCSFTNLREQYIRRLIMTSFIGYVFKALQEWMPSYSKPTHTTKTLLSELITLVDTLKQETNDVPSESVVNTILSIADSCKTQTQKSKEAKTTIDSFLREHFVFDPNLHAQSAYTCADTNVDTCASMCADTNVDTCASMCADTNVDTCASTCTSTEYTDLADPERIPLHIMQKTLNVPNELQADIDAITQTPQGYRAAAHILQNIELHQSIKHMLENPRAFKPILFNTKITRYLSQHIPPQDTFYKWNYYIEDNYEELRAATESIYPEKPDLEFAFIIYDVVDSSNQQKVDEFYYKYKDQIFSEVSSIQLGNWTLLGSFKANRERYNYFNQNNEIIKRILDRHEEDLKIGKEILRNTIYHKKAKNIQETGPDAPGLSIYNSTFHTDSGIKGLLSFKELKNLEKASGNIKKAREYDFIDDCEEKIKQLLSKENLTPDEESELIKTKKQLDNALEMLNVPDDTIRVDMWVNNNNKLEKEILYTKAEL

SEQ ID NO: 339 - protein, 385 aa, African swine fever virus
MDEIINKYQAVEKLFKEIQQGLAAYDQYKTLISEMMHYNNHIKQEYFNFLMIISPYLIRAHSGETLRNKVNNEIKRLILVENINTKISKTLVSVNFLLQKKLSTDGVKTKNMWCTNNPMLQVKTAHNLFKQLCDTQSKTQWVQTLKYKECKYCHTDMVFNTTQFGLQCPNCGCIQELMGTIFDETHFYNHDGQKAKSGIFNPNRHYRFWIEHILGRNPEQELGTKQDPCGTKVLQQLKKIIKRDNKCIALLTVENIRKMLKEINRTDLNNCVSLILRKLTGVGPPQISESILLRGEYIFTEAIKIREKVCKKGRINRNYYPYYIYKIFDAILPPNDTTNRRILQYIHLQGNDTLANNDSEWESICMELPEIKWKPTDRTHCVHFF

SEQ ID NO: 340 - protein, 646 aa, African swine fever virus
MASGGAFCLIANDGKADKIILAQDLLNSRISNIKNVNKSYGKPDPEPTLSQIEETHLVHFNAHFKPYVPVGFEYNKVRPHTGTPTLGNKLTFGIPQYGDFFHDMVGHHILGACHSSWQDAPIQGTSQMGAHGQLQTFPRNGYDWDNQTPLEGAVYTLVDPFGRPIVPGTKNAYRNLVYYCEYPGERLYENVRFDVNGNSLDEYSSDVTTLVRKFCIPGDKMTGYKHLVGQEVSVEGTSGPLLCNIHDLHKPHQSKPILTDENDTQRTCSHTNPKFLSQHFPENSHNIQTAGKQDITPITDATYLDIRRNVHYSCNGPQTPKYYQPPLALWIKLRFWFNENVNLAIPSVSIPFGERFITIKLASQKDLVNEFPGLFVRQSRFIAGRPSRRNIRFKPWFIPGVINEISLTNNELYINNLFVTPEIHNLFVKRVRFSLIRVHKTQVTHTNNNHHDEKLMSALKWPIEYMFIGLKPTWNISDQNPHQHRDWHKFGHVVNAIMQPTHHAEISFQDRDTALPDACSSISDISPVTYPITLPIIKNISVTAHGINLIDKFPSKFCSSYIPFHYGGNAIKTPDDPGAMMITFALKPREEYQPSGHINVSRAREFYISWDTDYVGSITTADLVVSASAINFLLLQNGSAVLRYST

SEQ ID NO: 341 - protein, 125 aa, African swine fever virus
MAVYAKDLDNNKELNQKLINDQLKIIDTLLLAEKKNFLVYELPAPFDFSSGDPLASQRDIYYAIIKSLEERGFTVKICMKGDRALLFITWKKIQSIEINKKEEYLRMHFIQDEEKAFYCKFLESR

SEQ ID NO: 342 - protein, 115 aa, African swine fever virus
MGYTIQLDKDGDYCWDEDPTHHDPYMQANATSHVATSYATTSHAATPHAAAHHTFHEPFIKLNLTDKNIFNGLGFILIVIFIYLLLITLQQMLTRHIYNTVQHCVKAHLDSKNLQ

SEQ ID NO: 343 - protein, 412 aa, African swine fever virus
MEDTTFLEGANLAGITTLMNNLHINEQANLEELEKQVMGKQQSFPTDHFDEELNGLAKSLGINFNDPEFSLDSPHSIISKKPSGRGGDKVHGGIRRDSVCTDSICSDSVCSGSIRSGSIRSGSIRNGSIRSGSVRDGSIRNGSVRSGKTRRGLACNSSSRNDRGYSLSTHRKKYAESEASQKTAISKRDRKNHYAESEYSEKSIKPSTKQVDRLINHLRSNGDPNSFYKKDHDYERKTKLVKLEKINMLLTYLGNEQISTDDIKIPTIDSSMQEIDDVIEMLTLRNVGIRYSSIAEEILIGLARGLEIVFDGTREIPFLNYRPDYTGLHNTFMIKLFKMRYETSQVVGNLVQNMSPLSKICLELGPSLLLYPALIRTKHKASEDLYNLLQKGPEDPFTAYNEIHETLKKNNK

SEQ ID NO: 344 - protein, 175 aa, African swine fever virus
METNCPNILYLSGITIEECLQSKKTATDTLNTNDDEAEVEKKLPSVFTTVSKWVTHSSFKCWTCHLYFKTVPKFVPTYMRENERGEIEMGVLGNFCSFSCAASYVDVHYTEPKRWEARELLNMLYRFFTSQWISYIKPAPSYTMRKEYGGKLSEEAFISELHTLEESISSKHIFI

SEQ ID NO: 345 - protein, 263 aa, African swine fever virus
MEDETELCFRSNKVTRLEMFVCTYGGKITSLACSHMELIKMLQIAEPVKALNCNFGHQCLPGYESLIKTPKKTKNMLRRPRKTEGDGTCFNSAIEASILFKDKMYKLKCFPSTGEIQVPGVIFPDFEDGKNIIQQWVDFLQHQPIEKKIQIIEFKTIMINFKFQINPVSPRVIIHLKKFAALLEHIPTPYPIREIKPPLEDSKVSAKFMVSPGKKVRINVFLKGKINILGCNTKESAETIYTFLKDLISVHWQEILCVLPVPD

SEQ ID NO: 346 - protein, 66 aa, African swine fever virus
MDIKRALILFLLFLVVLSNAFVDYIISNFNHAVTCRKPTYFGIVLQGIFLVILFSIVDYLINENIL

SEQ ID NO: 347 - protein, 1340 aa, African swine fever virus
MDFQNDFLTNPLRVTLYNPVENEYTKTFIFLGSVPANVLQACRKDLQRTPKDKEILQNFYGEDWEKKLSQYVVGGDSDDLDEFEKLFVEDRGEETNVMMPEIETMYSEYSIFPEDTFKDIREKIYVATGIPPYRQHIFFFQNNALQVTYRLLLSGSGVALDIRDYKKEFQQVGGVNIDASMESQKDELYVEALDSFQLIKNIHHIFVADLNTLVAPMRRQISIAIEDNYQFDLLYYGLIMKYWPLLSPDAFKLLVQSPLQMEKQYPALSPSLTSLKKRLLLEQKLINFTYARAQQVIAKYEGNRLTRGTLAVTSAMIKISPLVNIQINVRNVFDLFPATPDIPQLVVFFYSKTGPTVVSKHHITSTEPEKFSNKTFRVPTIILIRFINKKAFILTIQNNGHYFIESNWSENERHDFNSVVSTLNNFINPIIHTINDMGPAAFPRGGSLPLPSNEDIQISISSMSVSTFWPYTLSSKGFTELKSRWREYEQAGIISVRGLQQTGVYNFLFKKGIYSYDPHEIERMIIISSGPGRKMDINVALLQNTYAYLFDTNVAARWETIYGGRNIRIYHRVTDIKIEMFNITQEEFNYLWVYLFVFLDNLITGPDKILVNKLSQLHDKQQGKGASQLRALQEQDPDLYDLRKYDTQATVYSVLCQHPRPPVIYSEAEVKSMPPAKRKELVKYWNFTEGVPAYYSCPHPDYPHLSLLEGRHPLNYCLPCCQKTKALLGTKRFYINNTCLTKHTFVEQDLEDLNTQTSRHTLSYGKKIPVNRIAFLPHQIADELFLNTIKEPDIFCIVGVEQTMLGISNAGLFYSLARILDLAPKALAIEIAKAANTPQYYILGNGAGNMFSSGAELANLILQTFVEQKNQLLQWDTTWQDIFLDLVAICYDLHCVFFKDKQGDIGFEVSPSTIQKILSPSKKIAIIFDTDEGIYPMAITQQKRFLKNSEAQYIFTEDDPVMEVIQSMSEFMCKDNWWDIHDVKNIPGYTVGKKLINRHNFCYALLIDSDTDRPIYFPIRLSSYIHDDIPIDFDLRPTQIASFEETWKFITLFNKQYKQYEIVPSAVLQNIKKEFVGFLSEGKTGLYFYYAPTQTLPATLEKLPIATLTIDPRDIDQAILYPLEEPYPQQNKANKAFYINHLYKFLLIEFFDVLYGLQSNSTRKHIENLFQKTDFQKITSVTEFYTKLSDFVDLNDIHTIKHILETTDAEHALKVLQKNIFNFDYTLLSPLQSYTYDELCQHLKKLLTPRIEFYEDIETIDRGLINIYTSCQYSTLNQPQCKKKRLRIPVNHFENYIHILAADILNPLKHSTLLLTGLGVIDDLQFILRPQEIISVKNKF

SEQ ID NO: 348 - protein, 1206 aa, African swine fever virus
MISIMDRSEIVARENPVITQRVTNLLQTNAPLLFMPIDIHEVRYGAYTLFMYGSLENGYKAEVRIENIPVFFDVQIEFNDTNQLFLKSLLTAENIVYERLETLTQRPVMGYREKEKEFAPYIRIFFKSLYERRKAITYLNNMGYNTAADDTTCYYRMVSRELKLPLTSWIQLQHYSYEPRGLVHRFSVTPEDLVSYQNDGPTDHSIVMAYDIETYSPVKGTVPDPNQANDVVFMICMRIFWIHSTEPLASTCITMAPCKKSSEWTTILCSSEKNLLLSFAEQFSRWAPDICTGFNDSRYDWPFIVEKSMQHGILEEIFNKMSLFWHQKLDTILKCYYVKEKRVKISAEKSIISSFLHTPGCLPIDVRNMCMQLYPKAEKTSLKAFLENCGLDSKVDLPYHLMWKYYETRDSEKIADVAYYCIIDAQRCQDLLVRHNVIPDRREVGILSYTSLYDCIYYAGGHKVCNMLIAYAIHDEYGRIACSTIARGKREHGKYPGAFVIDPVKGLEQDKPTTGLDFASLYPSLIMAYNFSPEKFVASRDEANSLMAKGESLHYVSFHFNNRLVEGWFVRHNNVPDKMGLYPKVLIDLLNKRTALKQELKKLGEKKECIHESHPGFKELQFRHAMVDAKQKALKIFMNTFYGEAGNNLSPFFLLPLAGGVTSSGQYNLKLVYNFVINKGYGIKYGDTDSLYITCPDSLYTEVTDAYLNSQKTIKHYEQLCHEKVLLSMKAMSTLCAEVNEYLRQDNGTSYLRMAYEEVLFPVCFTGKKKYYGIAHVNTPNFNTKELFIRGIDIIKQGQTKLTKTIGTRIMEESMKLRRPEDHRPPLIEIVKTVLKDAVVNMKQWNFEDFIQTDAWRPDKDNKAVQIFMSRMHARREQLKKHGAAASQFAEPEPGERFSYVIVEKQVQFDIQGHRTDSSRKGDKMEYVSEAKAKNLPIDILFYINNYVLGLCARFINENEEFQPPDNVSNKDEYAQRRAKSYLQKFVQSIHPKDKSVIKQGNVHRQCYKYIHQEIKKKIGIFADLYKEFFNNTTNPIESFIQSTQFMIQYFDGEQKVNHSMKKMVEQHATASNRAGKPAGNPAGNALMRAIFTQLITEEKKIVQALYNKGDAIHDLLTYIINNINYKIATFQTKQMLTFEFSSTHVELLLKLNKTWLILAGIHVAKKHLQAFLDSYNNESPSRTFIQQAIEEECGSIKPSCYDFIS

SEQ ID NO: 349 - protein, 122 aa, African swine fever virus
MPSTGTLVIIFAIVLILCIMLLFFYKTVEAGKSGVLPPPIPPPTPPPKKKYDHNEYMEKTDLEPEVKKNHRKWANEAEHLISSSVKGLENLDETAFLANHKGHGFRTFDHAKSLFKEFLKKY

SEQ ID NO: 350 - protein, 2476 aa, African swine fever virus
MGNRGSSTSSRPLPSSEANIYAKLQDHIQRQTRPFSGGGYFNGGGDKNPVQHIKDYHIDSVSSKAKLRIIEGIIRAIAKIGFKVDTKQPIEDILKDIKKQLPDPRAGSTFVKNAEKQETVCKMIADAINQEFIDLGQDKLIDTTEGAASICRQIVLYINSLTHGLRAEYLDVHGSIENTLENIKLLNDAIKQLHERMVTEVTKAAPNEEVINAVTMIEAVYRRLLNEQNLQINILTNFIDNILTPTQKELDKLQTDEVDIIKLLNDTNSVLGTKNFGKVLSYTLCNLGIAASVANKINKALQKVGLKVEQYLQSKNWAEFDKELDLKRFSGLVSAENIAEFEKAVNLLRQTFNERHKILENSCAKKGGDEEKTPLDRRIEAQRLDRKHILMEFLNKSTQAYNDFLENVKKIGIKLVKEIALTPNITRLRDALSRINDMGTIALDLSLIGFYTNAAAREERETFLTQLTLVKNVLEEISKTDPNFKNLYDSCSRLLQIIDFYTDIVQKKYGGEEDCECTRVGGAALTVEELGLSKAARSQVDLNQAINTFMYYYYVAQIYSNLTHNKQEFQSYEENYATILGDAIAGRLMQLDTEKNARINSPAVDLARGHVGPNPGGAQEVDWKATVSAIELEYDVKRRFYRALEGLDLYLKNITKTFVNNIDSIQTVQQMLDGVRIIGRWFTETTGDTLAQVFESFPTSTGNDSNVFTDNAPAGHYYEKVAAEIQQGRSVGTLRPVRASQAKNIRDLIGRSLSNFQALKNIINAFARIGDMLGGEELRQMVPMSPLQIYKTLLEYLQHSALSVGLKNLNQSEIGGQRMALAQTAEEAAQRVYLSTVRVNDALSTRWETEDVFFTFMLKSMAAKIFIVLGIYDMFERPEPVYKLIPTRMILGGADELEPEVIPEAAELYFRLPRLAEFYQKLFSFRDENVQISMLPELEGIFSGLIRIIFMRPIELINIGDYSETEIRQLIKEINVIYQHFNLEYGEQEATKKALIHFVNEINRRFGVITRTEWEKFQRIVQEARTMNDFGMMNQTNYSILPDEDGYTQSSQLLPSDRFISPSTQPTPKWRPALYNIDSVDVQTGMLQPNSQWDLVQKFRKQLSEMFEDPSLQQELGKISYQELIRQAINELKKEHTDKIQIVSKLIQGSESLADTDVNKIFLFHETVITGLNLLSAIYVLLNNFRNNIKGLDLDTIQKSIIEWLRETQAANVNRANLIDWLGRKHGAISEIRNPGLVIKEINMRLSMVYPDPTTEAAAAAQDRNLTTETLFAWIVPYVGIPAGGGVRPEQELAARYLVDNQRIMQLLLTNIFEMTSSFNKMVQVRFPETSTAQVHLDFTGLISLIDSLMADTKYFLDLLRPHIDKNIIQYYENRSNPGSFYWLEEHLIDKLIKPPTDAGGRPLPGGELGLEGVNQIINKTYTLLTKPYNVLQLRGGAQRRDAANIQINNNPQSSERFEQYGRVFSRLVFYDALENNSGLRVEQVALGDFRLSNLIRTNNAQEENTLSYWDNIALRTYANVNDAANNLRRYRLYGSDYGIQNNRSMMMVFNQLIASYITRFYDAPSGKIYLNLINAFANGNFSQAVMEMGYAHPDLARNNNVFGHRGDPTEQSVLLLSLGLILQRLIKDTNRQGLSQHLISTLTEIPIYLKENYRANLPLFNKMFNILISQGELLKQFIQYTNVQLARPNLTALLGANNDSVIYYNNNNVPATGLSVGQAALRGIGGVFRPNVTLMPLGDAQNNTSDVVRKRLVAVIDGIIRGSHTLADSAMEVLHELTDHPIYLETEEHFIQNYMSRYNKEPLMPFSLSLYYLHDLRIENNEVYDPLLYPNLESGSPEFKLLYGTRKLLGNDPVQLSDMPGVQLIMKNYNETVVAREQITPTRFEHFYTHAIQALRFIINIRSFKTVMMYNENTFGGVNLISENRDDKPIITAGIGMNAVYSLRKTLQDVISFVESSYQEEQINHIHKIVSPKGQTRTLGSNRERERIFNLFDMNIIPINVNALMRSIPLANIYNYDYSFEEIACLMYGISAEKVRSLDTTAPQPDVAEVLNIPNRPPINTREFMLKLLINPYVSVSITQYGNELLSKGNAGYMSRIFRGDNALNMGRPKFLSDQIFNKVLFGSLYPTQFDYDEAGPSLAAGIQRGRERWGHPMSIYINQALHEIVRTIRLAETVRGLRNVIDRNQIIGELNAFRTQLEDTRREVNNLIQTPEIQNNPTPEIIAAIQNWVQQYRGQITNLIDLIGNAGQANSMINLIQNITPQTAGAQLTALFNIRGLPAPPPRQALQNDIEAMQWFMTMVINHPPVLIAPFMLLVNNLKEFLNTLERYVYKTPRWLGPGTARIAQPPVGMAPGINMRHHTSYTENSVLTYITEQNREEGPWSIVKQVGVGIQKPTLVQIGKDRFDTRLIRNLIFITNIQRLLRLRLNLELSQFRNVLVSPDHIINPSITEYGFSITGPSETFSDKQYDSDIRIL

SEQ ID NO: 351 - protein, 201 aa, African swine fever virus
MDFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKISFYLTYI

SEQ ID NO: 352 - protein, 530 aa, African swine fever virus
MPSNMKQFCKISVWLQQHDPDLLEIINNLCMLGNLSAAKYKHGVTFIYPKQAKIRDEIKKHAYSNDPSQAIKTLESLILPFYIPTPAEFTGEIGSYTGVKLEVEKTEANKVILKNGEAVLVPAADFKPFPDRRLAVWIMESGSMPLEGPPYKRKKEGGGNDPPVPKHISPYTPRTRIAIEVEKAFDDCMRQNWCSVNNPYLAKSVSLLSFLSLNHPTEFIKVLPLIDFDPLVTFYLLLEPYKTHGDDFLIPETILFGPTGWNGTDLYQSAMLEFKKFFTQITRQTFMDIADSATKEVDVPICYSDPETVHSYANHVRTEILHHNAVNKVTTPNLVVQAYNELEQTNTIRHYGPIFPESTINALRFWKKLWQDEQRFVIHGLHRTLMDQPTYETSEFAEIVRNLRFSRPGNNYINELNITSPAMYGDKHTTGDIAPNDRFAMLVAFINSTDFLYTAIPEEKVGGNETQTSSLTDLVPTRLHSFLNHNLSKLKILNRAQQTVRNILSNDCLNQLKHYVKHTGKNEILKLLQE

SEQ ID NO: 353 - protein, 80 aa, African swine fever virus
MLIPVVCFTCGFPIGTYAAIFDKARTEYIKTKMGGTLPQNIPLDASLQIELKDLITALGIPMRVCCRTHLITTLDYRKYY

SEQ ID NO: 354 - protein, 307 aa, African swine fever virus
MTTHIFHADDLLQALQQAKAEKNFSSVFSLDWDKLRTAKRNTTVKYVTVNVIVKGKKAPLMFNFQNEKHVGTIPPSTDEEVIRMNAENPKFLVKKRDRDPCLQFNKYKISPPLEDDGLTVKKNEQGEEIYPGDEEKSKLFQIIELLEEAFEDAVQKGPEAMKTKHVIKLIQRKISNSAVKNADKPLPNPIARIRIKINPATSILTPILLDKNKPITLQNGKTSFEELKDEDGVKANPDNIHKLIESHSIHDGIINARSICISNMGISFPLCLEMGVVKVFEKNNGIDVNSIYGSDDISTLVNQIAIA

SEQ ID NO: 355 - protein, 174 aa, African swine fever virus
MLTLIQGKKIVNDLRSRLAFEYNGQLIKILSKNIVAVGSLRREEKMLNDVDLLIIVPEKKLLKHVLPNIRIKDLSFSVKVCGERKCVLFIEWKKNTYQLDLFTALAEEKPYAVLHFTGPVSYLIRIRAALKKKNYKLNQYGLFKNQTLVPLKITTEKELIKELGFTYRIPKKRL

SEQ ID NO: 356 - protein, 61 aa, African swine fever virus
MALDGSSGGGSNVETLLIVAIIVVIMAIMLYYFWWMPRQQKKCSKAEECTCNNGSCSLKTS

SEQ ID NO: 357 - protein, 1460 aa, African swine fever virus
MKFYYVFSKNMEAGYAEIAAVQFNIAGDNDHKRQGVMEVTISNLFEGTLPAEGGIYDARMGTTDHHYKCITCSHQRKQCMGHPGILQMHAPVLQPLFIAEIRRWLRVICLNCGAPIVDLKRYEHLIRPKRLIEAASSQTEGKQCYVCKAVHPKIVKDSEDYFTFWADQQGKIDKLYPQIIREIFSRVTYDTVVKLGRSKNSHPEKLVLKAIQIPPISIRPGIRLGIGSGPQSFHDINNVIQYLVRKNLLIPKDLQIVRGQKIPLNIDRNLQTIQQLYYNFLLDSVSTTATQGGTGKRGIVMGARPAPSIMRRLPRKEGRIRKSLLGSQVWSISRSTICGNSDLHLDEVGYPISFARTLQVAETVQHYNINRLMPYFLNGKRQYPGCSRVYKQITQSVHDIEGLKQDFRLEVGDILYRDVVTGDVAFFNRQPSLERSSIGVHRIVVLENPKISTFQMNVSACAWYNADFDGDQMNLWVPWSVMSRVEAELLCSVRNWFISTKSSGPVNGQVQDSTVGSFLLTRTNTPMGKNVMNKLHAMGLFQTTQTDPPCFANYSPTDLLDGKSVVSMLLRQTPINYQRAPTWYSEVYAPYMHYNKQDISTQIRNGELIEGVLDKKAVGAGSSGGIYHLISRRYGPQQALKMIFATQQLALNYVRNAGFTVSTADMLLTPEAHQEVQEIINELLLESEEINNRLLHGDIMPPIGLTTHDFYEKLQLNALKFPDRILKPIMNSINPETNGLFQMVATGAKGSNPNMIHIMAGIGQIEINTQRIQPQFSFGRTLVYYPRFALEAQAYGFICNSYIAGLTSPEFIFGEMNGRFDLINKALSTSSTGYANRKAIFGLQSCIVDYYRRVSIDTRLVQQLYGEDGLDARQLETVRFETIMLSDQELEDKFKYTGIQSPLFEEEFSRLKKDRDKYRQIFLNVENFNFSQLLTDVRQVPVNVASIVKNILLSSTSGVLPFDEKSILQKYAMVKTFCKNLPYVFINNIQERLQTPIPVYLKRAASLMRMLIRIELATVKTLNITCEQMSAILDLIRLQYTQSLINYGEAVGILAAQSVSEPLTQYMLDSHHRSVAGGTNKSGIVRPQEIFSAKPVEAEQSSEMLLRLKNPEVETNKTYAQEIANSIELITFERLILQWHLLYETYSSTKKNVMYPDFASDVEWMTDFLENHPLLQPPEDIANWCIRLELNKTTMILKSISLESIINSLRAKHPNTYIMHSVENTASGIPIIIRIYLRESAFRRSTNTRMATDEKIAVNVVDKLLNSTIRGIPGIKNANVVKLMRHRVDAQGKLVRLDNIYAIKTNGTNIFGAMLDDNIDPYTIVSSSIGDTMELYGIEAARQKIISEIRTVMGDKGPNHRHLLMYADLMTRTGQVTSLEKAGLNAREPSNVLLRMALSSPVQVLTDAAVDSAVNPIYGIAAPTLMGSVPRIGTMYSDIIMDEKYITENYKSVDSLIDML

SEQ ID NO: 358 - protein, 419 aa, African swine fever virus
MLNQFPGQYSNNIFCFPPIESETKSGKKASWIICVQVVQHNTIIPITDEMFSTDVKDAVAEIFTKFFVEEGAVRISKMTRVTEGKNLGKKNATTVVHQAFKDALSKYNRHARQKRGAHTNRGIIPPMLVKYFNIIPKTFFEEETDPIVQRKRNGVRAVACQQGDGSILLYSRTKKEFLGLDNIKKELKQLYLFIDVRVYLDGELYLHRKPLQWIAGQANAKTDSSELHFYVFDCFWSDQLQMPSNKRQQLLTNIFKQKEDLTFIHQVENFSVKNVDEALRLKAQFIKEGYEGAIVRNANGPYEPGYNNYHSAHLAKLKPLLDAEFILVDYTQGKKGKDLGAILWVCELPNKKRFVVTPKHLTYADRYALFQKLTPALFKKHLYGKELTVEYAELSPKTGIPLQARAVGFREPINVLEII

SEQ ID NO: 359 - protein, 868 aa, African swine fever virus
MASLDNLVARYQRCFNDQSLKNSTIELEIRFQQINFLLFKTVYEALVAQEIPSTISHSIRCIKKVHHENHCREKILPSENLYFKKQPLMFFKFSEPASLGCKVSLAIEQPIRKFILDSSILVRLKNRTTFRVSELWKIELTIVKQLMGSEVSAKLAAFKTLLFDTPEQQTTKNMMTLINPDDEYLYEIEIEYTGKPESLTAADVIKIKNTVLTLISPNHLMLTAYHQAIEFIASHILSSEILLARIKSGKWGLKRLLPQVKSMTKADYMKFYPPVGYYVTDKADGIRGIAVIQDTQIYVVADQLYSLGTTGIEPLKPTILDGEFMPEKKEFYGFDVIMYEGNLLTQQGFETRIESLSKGIKVLQAFNIKAEMKPFISLTSADPNVLLKNFESIFKKKTRPYSIDGIILVEPGNSYLNTNTFKWKPTWDNTLDFLVRKCPESLNVPEYAPKKGFSLHLLFVGISGELFKKLALNWCPGYTKLFPVTQRNQNYFPVQFQPSDFPLAFLYYHPDTSSFSNIDGKVLEMRCLKREINHVSWEIVKIREDRQQDLKTGGYFGNDFKTAELTWLNYMDPFSFEELAKGPSGMYFAGAKTGIYRAQTALISFIKQEIIQKISHQSWVIDLGIGKGQDLGRYLDAGIRHLVGIDKDQTALAELVYRKFSHATTRQHKHATNIYVLHQDLAEPAKEISEKVHQIYGFPKEGASSIVSNLFIHYLMKNTQQVENLAVLCHKLLQPGGMVWFTTMLGEQVLELLHENRIELNEVWEARENEVVKFAIKRLFKEDILQETGQEIGVLLPFSNGDFYNEYLVNTAFLIKIFKHHGFSLVQKQSFKDWIPEFQNFSKSLYKILTEADKTWTSLFGFICLRKN

SEQ ID NO: 360 - protein, 250 aa, African swine fever virus
MDTAMQLKTSIGLITCRMNTQNNQIETILVQKRYSLAFSEFIHCHYSINANQGHLIKMFNNMTINERLLVKTLDFDRMWYHIWIETPVYELYHKKYQKFRKNWLLPDNGKKLISLINQAKGSGTLLWEIPKGKPKEDESDLTCAIREFEEETGITREYYQILPEFKKSMSYFDGKTEYKHIYFLAMLCKSLEEPNMNLSLQYENRIAEISKISWQNMEAVRFISKRQSLNLEPIIGPAFNFIKNYLRYKH

SEQ ID NO: 361 - protein, 129 aa, African swine fever virus
MDINPLLYLQAFNNDATTFNTQGHILEQQSDSAYFDTFANAMQAYLDTKQGGNDEEGTIILMDDEDFNDSESLEDFLQMLSEEELNDGFSSDDEPEEHVILTEDNQGEPSETPQATFDITEFIKTDDED

SEQ ID NO: 362 - protein, 79 aa, African swine fever virus
MNKTIEYQKEFLKENNQLLSIPVKKNILKEILQNDEQDTIITNCITKEVSINLDLIKNPKVLYSIYIMVVEYLKSINIA

SEQ ID NO: 363 - protein, 339 aa, African swine fever virus
MIDQKIFETTLNIDDPTNFCTNVEAHLLKELENIYVGKCFKNSFILNITGVIQRSPCFIMRTNNSGRGYMHVRFSAVVSYLNAFDLIAAVKIIKNDSNIILGESLLTEPVTIVIPSSESQNNVAEVGQIVPVQLANSSVYYIPGRQQASATGSIFIPKHTFSVYHVQEELTQEQALNLTKLVNIIEMLLESRSKKDFKQICFFEKLYYTYSISSDEILDLKIWKGPKGKEMSRLKPCNVLSFLYDALKNKNSSLGFWARPPNLLKSSPLAYQQDQNSFNATELPIICSAEVMFVTLLKEIINYLQFINDLCDTFNNEQLIKRHENIWMLIEQRKIGHDF

SEQ ID NO: 364 - protein, 1133 aa, African swine fever virus
MAYPELDAADFLQQLARRKEFKSLISPPVDQKELIRDLRAHFVQIGGPGCEKGGRAFFPCDPYASPFPSIKGLQLHNAQLFVQNFQNPNTPYSRLLLNWQTGTGKSIAAIAIARQFMNHYMNFIENAPWIFVVGFTRAIIQTEMLRRPELGFVSYKEVAELHRLLHIAKQSGSTTSVESRHLNGFVSTLKRRLTDRNRGGFFQFYGYKEFASKLFNITSKGEEKNFDVLSLFHRSDEAEDTLNENDISQFVQKISEAETNGLIRVNQKIMEQLRGGLLIADEIHNVYNIQERNNYGIALQYVLDAFPPHQAPRAVFMSATPVTGSVMEYVDLLNLLVPRHELPNGQPLQRQQLFDSSGHSVKWKKDALALVERLSTGRVSFLLDTNTNFYPERIFAGKMLSYKDETLPYLHFIECPMSEYQLETLKQLGPDPKISSNAYSIYDMVFPNPKFSKQTEPKAYGLFNSTETPTALSMASTDWLLENGVQIIEPSRRAPFNVSGSFLSLQPPTHISGLAFYSGKYTQMMKDILSIIRQGRGKILIYHNRVRMSGVLILQEILQSNGILNEVSSPVGTTRCSICAAIRDEHTHSDHQFIPVRFTILHSEIEPAVRERSLALFNASSNLEGHQLRILIGSKVIVEGLNFQAVRYEMIMSLPLDIPRLIQVFGRVVRKNSHMELPPSERNVTIYLYVSTTPDGGPELAKYAQKLKEYILIQEGDKALRKHAIDGFTNQIKIDKPMLESLPLSPSITPANVGATVLNTFEAYGYGEQEVKTISNIIISLFMARPVWTYSELWKAVSTPKLIQGITIDNKLFSEDNFALALISLCYSKNQCKELWIQNRLCTIMHVPAKPEHLYVAAVLNHKKEPVLDIETYIRDFQPPAMHSIRITKYLEHSQTKEPFQVLYEKFQKDFQDEPMEQVLIHYPASFHYTMLEALIIDNLAGMGALVEVYKKFFIAFSKKDIQPFPDIFKIISHVPGDDNTLVGYATEDSVRLITSREDKTWHEIPLYMLNINVKRKENDIVIGYMESKGKALKFKIRPPIQVLKKNEITDIRMLNRGAVCETRGREEQQKIADQLGISLNLTKISAIKLCLLIRNNLLQKEMEARNQPNGMQDGIRWFYLFNDKMPSLVHTS

SEQ ID NO: 365 - protein, 117 aa, African swine fever virus
MDTETSPLLSHNLSTREGIKQSTQGLLAHTIAKYPGTTAILLGILILLIIILIIVAIVYYNRTIDCKSSIPKPPPSYYVQQPEPHHHFPVFFRKRKNSTSLQSHIPSDEQLAELAHS

SEQ ID NO: 366 - protein, 205 aa, African swine fever virus
MAMQKLFTYIYEFIEYRKMVLLEEKVPYDKFVQMVLNTGFFRINAETLNHGIVSVFIFGANGKYVHHGGDMRTLLTNTLNEKKHYEELILIVDKPVLSKKNILDIIVEQRAANPTIVINIYPYHLFCINIPKVSAIPKHKLITQEEAQEFLGREYLQPQDLMQISASDPPVVWLGGRPGDFVQIERPSETAMHAVVIRFITKSKI

SEQ ID NO: 367 - protein, 345 aa, African swine fever virus
METFVRLFKDSPQQRSDAWHAIRRTQVGGSDLASVLGLNPYKSYYITLAEKANLFKKNLNRAACSWGTLFERVSKDLLELFCQTTVIGDNIHIDGTYLGYPGHSNSPDGFCHLTLGYTQQSWEIKTIFNNVRYEATKRIPVLVEIKSPFNRKIKNSVPSYYMPQIQSGLALSPPISMGIYVEAMFRVCGIHQLGSNNETNTDIHPPESMLPLAWGIITICSTQEHTEAPQDFGTLDAETFRQLLETLYQKDQYTIHYSMPYETACPEMPNVVGYFGWKVFIFQIIPVMKHPQFLKDKYPIIQQFLRDLHTIKASPSPMEMYEKICCSEESALSTEDIDNFTDMLT

SEQ ID NO: 368 - protein, 183 aa, African swine fever virus
MSVVVGGVEYSLNNWARYEIKRRAAELESVNYYPHCEYIMPEDIVVSILGSKPNCPFLEALKRFHDFLKKRRIIFKGEYLVIPWMGAQDVADMIHHVENRINLDHLEDLAHMLKLITYHRSFDTCINQAFEHLYAFKFPDANIETHELKHIRQLEKKMYGYILRLEKLQTVLTFYIEFLLKQV

SEQ ID NO: 369 - protein, 273 aa, African swine fever virus
MSILEKITSSPSECAEHLTNKDSCLSKKIQKELTSFLEKKETLGCDSESCVITHPAVKAYAQQKGLDLSKELETRFKAPGPRNNTGLLTNFNIDETLQRWAIKYTKFFNCPFSMMDFERVHYKFNQVDMVKVYKGEELQYVEGKVVKRPCNTFGCVLNTDFSTGTGKHWVAIFVDMRGDCWSIEYFNSTGNSPPGPVIRWMERVKQQLLKIHHTVKTLAVTNIRHQRSQTECGPYSLFYIRARLDNVSYAHFISARITDEDMYKFRTHLFRIA

SEQ ID NO: 370 - protein, 1192 aa, African swine fever virus
MEAFEISDFKEHAKKKSMWAGALNKVTISGLMGVFTEDEDLMALPIHRDHCPALLKIFDEIIVNATDHERACHNKTKKVTYIKISFDKGVFSCENDGPGIPIAKHEQASLIAKRDVYVPEVASCHFLAGTNINKAKDCIKGGTNGVGLKLAMVHSQWAILTTADGAQKYVQHINQRLDIIEPPTITPSREMFTRIELMPVYQELGYAEPLSETEQADLSAWIYLRACQCAAYVGKGTTIYYNDKPCRTGSVMALAKMYTLLSAPNSTIHTATIKADAKPYSLHPLQVAAVVSPKFKKFEHVSVINGVNCVKGEHVTFLKKTINEMVVKKFQQTIKDKNRKTTLRDSCSNIFIVIVGSIPGIEWTGQRKDELSIAENVFKTHYSIPSSFLTSMTKSIVDILLQSISKKDNHKQVDVDKYTRARNAGGKRAQDCMLLAAEGDSALSLLRTGLTLGKSNPSGPSFDFCGMISLGGVIMNACKKVTNITTDSGETIMVRNEQLTNNKVLQGIVQVLGLDFNCHYKTQEERAKLRYGCIVACVDQDLDGCGKILGLLLAYFHLFWPQLIIHGFVKRLLTPLIRVYEKGKTMPVEFYYEQEFDAWAKKQTSLANHTVKYYKGLAAHDTHEVKSMFKHFDNMVYTFTLDDSAKELFHIYFGGESELRKRELCTGVVPLTETQTQSIHSVRRIPCSLHLQVDTKAYKLDAIERQIPNFLDGMTRARRKILAGGVKCFASNNRERKVFQFGGYVADHMFYHHGDMSLNTSIIKAAQYYPGSSHLYPVFIGIGSFGSRHLGGKDAGSPRYISVQLASEFIKTMFPAEDSWLLPYVFEDGQRAEPEYYVPVLPLAIMEYGANPSEGWKYTTWARQLEDILALVRAYVDKDNPKHELLHYAIKHKITILPLRPSNYNFKGHLKRFGQYYYSYGTYVISEQRNIITITELPLRVPTVAYIESIKKSSNRMTFIEEIIDYSSSETIEILVKLKPNSLNRIVEEFKETEEQDSIENFLRLRNCLHSHLNFVKPKGGIIEFNTYYEILYAWLPYRRELYQKRLMREHAVLKLRIIMETAIVRYINESAELNLSHYEDEKEASRILSEHGFPPLNHTLIISPEFASIEELNQKALQGCYTYILSLQARELLIAAKTRRVEKIKKMQARLDKVEQLLQESPFPGASVWLEEIDAVEKAIIKGRNTQWKFH

SEQ ID NO: 371 - protein, 359 aa, African swine fever virus
MEKIFQNVEIKPFLIDFSNLFIKNAAKKLFQLEEQLPLVPVNVVMDFKGISRAAVHGLSRVLQDEIPNYMLDIKPGGYKIEDSTDLFMTEQFIRNRINFIPIYAKNETLVFALRSLNNSCEVKTIYSRDLIQVAGPKLKYPIFNPTFEIGFLQPGKSLIIEDIYIKKGIGRKHAAFNLAVKTHFSHLDIEQYPTDKKEYMALSGYKQSSMTSDPRHHRLGLCFPAVPLPHINQAVRTYLKNACRIIIGRIQSIQKIYENFEEPQPELVLFSMDEEKTKAIITIKDETHTIGNLLKTYIYEMIPDISFVGYQCVPHKQEMVLTIIHKASQEDLITLLEKSIQNIIQTFQILEKNVDELIA

SEQ ID NO: 372 - protein, 171 aa, African swine fever virus
MVVYDLLVSLSKESIDVLRFVEANLAAFNQQYIFFNIQRKNSITTPLLITPQQEKISQIVEFLMDEYNKNNRRPSGPPREQPMHPLLPYQQSSDEQPMMPYQQPPGNDDQPYEQIYHKKHASQQVNTELNDYYQHILALGDEDKGMDSMLKLPEKAKRDSDDEDDMFSIKN

SEQ ID NO: 373 - protein, 124 aa, African swine fever virus
MNLEYVQVVQKFNQVLLELTKKVCTVVGGSKPTYWYHHIRRVCSECPSMPMSMIGPYLNVYKAQILTRDKNFFMNFDPAHNEYTFIIQKLKEAARNMPEDELEQYWVKLLFLLKSYIKCKPFIN

SEQ ID NO: 374 - protein, 339 aa, African swine fever virus
MAGRVKIKQKELIDSTVKNKNVMNLFHEIIGSKGNINFSVVWPKFKKIKQSVYDYISTLSVLEKANVMQNFEADKKLLELFVQKLWAAYEGYFKYPEIEKYEVEGQVNFNLVPQCVLEKFSQLYRIRINSELVTLILNSCAFMSKYNDYILKKDPYILTITPGLCFSPIPNFEDLNFKHLYNSDKNSQHDKEFIMFILYKLYTAALGVYNAISIPDIDVEDLENIILSSVSQIKKQIPRCKDAFNKIESSVHLLRKNFNTYYSDYVGSGYNPTIIMEQYIKDISQDSKNISPRISYQFRTIIKYYRDMIATRHQTMDPQVLNLVKHVEKKLDMLDREKN

SEQ ID NO: 375 - protein, 108 aa, African swine fever virus
MVNLFPVFTLIVIITILITTRELSTTMLIVSLVTDYIIINTQYTEQQHENNTFFMPQKNSFNESYNKDKKSNIHIPYQWLAPELKEAESKYWWGNYDPHSEPVLAGAS

SEQ ID NO: 376 - protein, 233 aa, African swine fever virus
MILIASPFSLAHLEYLHTWHVTIKNIAQQHGLDIKVAIVVSTSHLNNFLPISGALNIECITFPSCGIKEIDLLWARIKLFQHYCAIGARLLWLVSADIRPPVSAWPAIADSLKKGADAVVIPYPSRWNNLIPTVIKEIVVHQKKCLVAVDARHLDTDTQIVGAGMGCIVLTLKALMVRLSIGKQPVKILWPDLHGTAEGIPLEGVEVGWFLNAYAHKLNIRCLGADHIAQHLT

SEQ ID NO: 377 - protein, 241 aa, African swine fever virus
MAANIIATRAVPKMASKKEHQYCLLDSQEKRHGHYPFSFELKPYGQTGANIIGVQGSLTHVIKMTVFPFMIPFPLQKTHIDDFIGGRIYLFFKELDMQAVSDVNGMQYHFEFKVVPVSPNQVELLPVNNKYKFTYAIPVVQYLTPIFYDLSGPLDFPLDTLSVHVDILSNHIQLPIQNHNLTTGDRVFISGYKHLQTIELCKNNKIFIKNIPPLSSEKIKLYILKNRIRIPLYFKSLKTSK

SEQ ID NO: 378 - protein, 298 aa, African swine fever virus
MSRPEQQLKKMLKNPQAQYAFYPTAKVERISTTQHMYFIATRPMFEGGRNNVFLGHQVGQPIIFKYVSKKEIPGNEVIVLKALQDTPGVIKLIEYTENAMYHILIIEYIPNSVDLLHYHYFKKLEETEAKKIIFQLILIIQNIYEKGFIHGDIKDENLIIDINQKIIKVIDFGSAVRLDETRPQYNMFGTWEYVCPEFYYYGYYYQLPLTVWTIGMVAVNLFRFRAENFYLNDILKRENYIPENISETGKQFITECLTINENKRLSFKSLVSHPWFKGLKKEIQPISELGVDYKNVIT

SEQ ID NO: 379 - protein, 706 aa, African swine fever virus
MSCVHNNTSFPVQIEAYLKEVYEKYKELQESKDTSLTARFARALKYYQFLIYTAFSDPKFGIGQGENTRGLLIYHQMGMGKTILSLSLAISLSHIYNPILIAPKSLHSNFQQSLLKLIKLLYPETTDHSKELQKISRRFRFVSLDAYNMGQQIIKAGGSLNGCLLIVDEAHNLFRGIINSANDKTNARQLYNNIMQAKNIRILFLTGTPCSKDPFEMVPCFNMLSGRILLPLHYERFYTAYVNKTTNSPLNADKLLNRLVGMISYAGNQNELNKLFPTELPLIIEKVEMSPEQYRQYLLARDVENAEKHASSGMYEKINAAALCLPGSEQESGSSYYVRSRMISIFASEMLTVKEDEKLSEAVQQLPKEAFTENSSPKIVRMLKNIKTSPGPVLIYSQFVELGLHVVARFLEIEGYQCLQPLKVLEEGHNTILLHKDGKDLMVKNFAEDGPTHTLVLSSKITRFTLITGKILSKERDMIQQLWNSPLNIHGEVIKILLVSKTGAEGLDLKYGRQVHILEPYWDKAREDQVKARIIRIGSHDALPPEEKTVQPFLYIAVANQKMFYSIPEGSQEQKTIDERFHERGLEKSHLNSAFRDLLKRAAIECAFNGESGCLMCQPTNALLFHENFERDLRLPNPCQPLVKAEVKAYSISYEGKQFFYQKNKDVGLGYTFYEYNPIIKAYIEIKPSNPLYIKLIKHVQAGTTA

SEQ ID NO: 380 - protein, 509 aa, African swine fever virus
MEAIISFAGIGINYKKLQSKLQHDFGRVLKALTVTARALPGQPKHIAIRQETAFTLQGEYIYFPILLQKQFEMFNMVYTTRPVSLRALPCVETEFPLFNYQQEMVDKIHKKLLSPYGRFYLHLNTGLGKTRIAISIIQKLLYPTLVIVPTKAIQIQWIDELTLLLPHLRVAAYNNAACKKKDMTSKEYDVIVGIINTLRKKPEQFFEPFGLVVLDEAHELHSPENYKIFWKIQLSRILGLSATPLDRPDGMDKIIIHHLGQPQRTVSPTTTFSGYVREIEYQGHPDFVSPVYINEKVSAIATIDKLLQDPSRIQLVVNEAKRLYSLHTAEPHKWGTDEPYGIIIFVEFRKLLEIFYQALSKEFKDVQIIVPEVALLCGGVSNTALSQAHSASIILLTYGYGRRGISFKHMTSIIMATPRRNNMEQILGRITRQGSDEKKVRIVVDIKDTLSPLSSQVYDRHRIYKKKGYPIFKCSASYQQPYSSNEVLIWDPYNESCLACTTTPPSPSK

SEQ ID NO: 381 - protein, 383 aa, African swine fever virus
MASILTLDGLYAEVPKFLPEALREGCAGKNPLSFYIQQILNLMGCDGNEYHVLFTSSSEEANTHMIMAAVRRHLLRTQQRPHVIIGAAEPPSVTECVKALAQEKRCVYTIIPLKNFEIDPVAVYDAIQSNTCLACISGTNAVVKTFNKLQDISNVLKGIPLHSEVSDLVYQGCIQQNPPADSFSINSLYGFLGVGVLGMKKKVMQGLGPLIFGGGLRGGSPNIPGIHAMYKTLTQQRPSMKKNKYNTYAVHENFKKHQHVYLPIGGVSAEDTSAENISTKDMPVEGPKGLPGYILFSVGRRAEELQKKIFTKFNIKVGRVVDLQEILFRIKIPQKYWETLLFIQLRDNLTKEDIKRVMVVLMHLDTITPRGSLPPPSHSSSFS

SEQ ID NO: 382 - protein, 184 aa, African swine fever virus
MKTFITCTSVKNYFRQHLKTNQRISSELISYVCTILNHICHQYLQNPQAQEEEWFALIKELPIIKDGLSKEERFFSSGVKHFLHEYKITPENQEKFQKMLNAITEQLMSRLCKVFSIMIQRQGFLKTQTLMYSHLFTILSILMVADNLYGEQDPTEFFSLIIEQTKTIKKKKKSGSEEEESHEE

SEQ ID NO: 383 - protein, 184 aa, African swine fever virus
MDSEFFQPVYPRHYGECLSPVTTPSFFSTHMYTILIAIVVLVIIIIVLIYLFSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASQTMSAIENLRQRNTYTHKDLENSL

SEQ ID NO: 384 - protein, 423 aa, African swine fever virus
MLWRNEITEFMDQLSKYSQEILKTFKQLRPSEYKQYNEFLTQVTPLLQKTPEKIPELVDHIFNYLDNVEKICELLVNASSIIISSKIREQVKHGMSFSYKADLDSLADILSQKQYVLMHLSKNIAAEYFNTCLNQGKSKLDLKAASVFYSSRSRTASSAELYRKMLYAYGSPQEINYYTEKARNKTLDVEESDSMAIIERTARHNLSLMHPLEAMGLTFGATNTDADPEDLKDKTVINLTLPQATESITYHLKSLMQLKKVSTASGLNTNILKAFDNIISTPVKKNKMASKLAPGMDVVFTSDNGKTFFTKNILSKNMLAGPKERVFAYNNLISNLNNSCFIQNHNDFLRQQDSWPFYDAHNFTNKFLMQPIFSGQTRPRLQGAMEAAHVETHLTAFLQSIQPSRPQDPSVLASPKLSALILN

SEQ ID NO: 385 - protein, 301 aa, African swine fever virus
MSEDIRRGPGRPPKKRVVPNFERKGILEKPVRPQSRLEFSYDNPLIFKNLFIYFKNLKSKNILVRCTPTEITFFSRDQSQASFVIATIDGKNVNHYYASDVFWLGINRELVEKMFNSIDRSFLKITIVHRYDKPETLFFIFTDFDIDKECTYQITVSEPELDMDLIEMEKSISEERLKNYPLRWEFTSKQLKKTFSDLSNYTELVTIEKLGGDTPLHLYFQKFNSISYHEMYKSSNKINLTSTIPKSQVFQINVKIAHIKSLASAMVTDKIRILCEENGNLIFQSEMDALMLNTITLNTTI

SEQ ID NO: 386 - protein, 146 aa, African swine fever virus
MGGTTDFVLSITIVLVILIIIAFIWYNFTGWSPFKYSKGNTVTFKTPDESSIAYMRFRNCVFTFTDPKGSLHSIDVTEVLNNMAKGFRDAQNPPSSFTLGGHCQAPLNAFSFVLPGVNDRATVATADEAKKWENCDATLTGLQRII

SEQ ID NO: 387 - protein, 199 aa, African swine fever virus
MSCMPVSTKCNDIWVDFSCTGPSISELQKKEPKAWAAILRSHTNQQTAEDDNIIGSICDKQGLCSKDEYAYSQYCACVNSGTLWAECAFAPCNGNKNAYKTTEQRNILTNKQCPSGLTICQNIAEYGGSGNISDLYQNFNCNSVINTFLINVMNHPFLTLILIILILIIIYRLMSSSGGKHNDDKLPPPSLIFSNLNNF

SEQ ID NO: 388 - protein, 165 aa, African swine fever virus
MATNFFIQPITEEAEAYYPPSVITNKRKDLGVDVYCCSDLVLQPGLNIVRLHIKVACEHMGKKCGFKIMARSSMCTHERLLILANGIGLIDPGYVGELMLKIINLGDTPVQIWAKECLVQLVAQGDHVPDHINILKRNQIFPLFAPTPRGEGRFGSTGEAGIMRT

SEQ ID NO: 389 - protein, 248 aa, African swine fever virus
MGGSTSKNSFKNTTNIISNSIFNQMQSCISMLDGKNYIGVFGDGNILNHVFQDLNLSLNTSCVQKHVNEENFITNLSNQITQNLKDQEVALTQWMDAGTHDQKTDIEENIKVNLTTTLIQNCVSSLSGMNVLVVKGNGNIVENATQKQSQQIISNCLQGSKQAIDTTTGITNTVNQYSHYTSKNFFDFIADAISAVFKNIMVAAVVIVLIIVGFIAVFYFLHSRHRHEEEEEAEPLISNKVLKNAAVS

SEQ ID NO: 390 - protein, 122 aa, African swine fever virus
MADFNSPIQYLKEDSRDRTSIGSLEYDENADTMIPSFAAGLEEFEPIPDYDPTTSTSLYSQLTHNMEKIAEEEDSNFLHDTREFTSLVPDEADNKPEDDEESGAKPKKKKHLFPKLSSHKSK

SEQ ID NO: 391 - protein, 296 aa, African swine fever virus
MFGAFVSHRLWSDSGCTTTCITNSIANYVAFGEQIGFPFKSAQVFIAGPRKAVINIQEDDKVELLKMIVKHNLWVVAHGTYLDVPWSRKSAFVTHFIQQELLICKEVGIKGLVLHLGAVEPELIMEGLKKIKPVEGVVIYLETPHNKHHTYKYSTIEQIKELFLRIRNTRLKQIGLCIDTAHIWSSGVNISSYNDAGQWLRSLENIHSVIPPSHIMFHLNDAATECGSGIDRHASLFEGMIWKSYSHKIKQSGLYCFVEYVTRHQCPAILERNLGSSMQLQTALTAEFTTLKSLLK

SEQ ID NO: 392 - protein, 111 aa, African swine fever virus
MSFSECPLVISACKKFLQKRITIENEALINALITALAQTSTLNDLCLLPIQTYLLSYKNAFEWIHFVCIAITTILDNKYNWKDCTVDINYIFLHVTYIYNIKTKEYLDYCS

SEQ ID NO: 393 - protein, 50 aa, African swine fever virus
MHISIITRYTLKYIYMHLTHNHILFTYIMRIYLIKHNHMLFTTHVYTYIM

SEQ ID NO: 394 - protein, 279 aa, African swine fever virus
MLLVLIDVDGFMGQLYNENGTQTILIPREVVIFYWEKNTPSKILQLFFHGGIDPIFEKINQRSFSFQSRHIHHFTLDESPLPNSIALPTDTLQAFKAGKKMIFQHLVKITKDHEQILLLHKGGPEGEWIRSFNIPNATVQNLNDLCCPSVEKLVLKKRDYISSSIGCPKHIQGSNHCPVFECHVLFKWIQENTSIVQGVLERPSLPYEKADLFIEHRINMVDNHPFKKDSIKQNQKKRTGSQRNLSNMGYMLTMASLARFIINTASFNKCIYPLSQDIR

SEQ ID NO: 395 - protein, 226 aa, African swine fever virus
MKMETFLVCLFHNADGLHQQIQEILYLLRMHIYETNLYLKQELSRLIYPNRQLSFVLLMPLSLLRNWDDIEYLTDVVDDKQTLHYAANLLTNYVLHLSMFQKLTKPYFLLAVKRVSEKLNKKQRHSFYEVLVTSETLNNYENLSKNILNTLMFAVRYVFKPTPNYSEILAELEKKNKIHHIIFNMVITDFAQIREQQMDKHLCETNNELRQECKETIFDLKVVGNV

SEQ ID NO: 396 - protein, 243 aa, African swine fever virus
MKMHIARDSIVFLLNKYLQNTILTNKIEQECFLQADTPKKYLQYIKPFLINCMTKNITTDLVMKDSKRLEPYIILEMRDIIQMMFFRTLQKHMFFKEHTDLCTEYAQKIEASCYHYTYQQQEKTFLEEYSTRCGTINHIINCEKKSHQQQDNDALNKLISGELKPEAIGSMTFAELCPSAALKEKTEITLRSQQKVAEKTSQLYKCPNCKQRMCTYREVQTRALDEPSTIFCTCKKCGHEFIG

SEQ ID NO: 397 - protein, 72 aa, African swine fever virus
METQKLISMVKEALEKYQYPLTAKNIKVVIQKEHNVVLPTGSINSILYSNSELFEKIDKTNTIYPPLWIRKN

SEQ ID NO: 398 - protein, 329 aa, African swine fever virus
MLRVFIFFVFLGSGLTGRIKPQVTCKYFISENNTWYKYNVTILNSSIVLPAYNTIPSNAAGISCTCHDIDYLQKNNISIHYNTSILKTFQDIRIIRCGMKNISEIAGGFGKELKFLDLRYNDLQVIDYNILRKLIRSNTPTYLYYNNLMCGKRNCPLYYFLLKQEQTYLKRLPQFFLRRINFSNNNTFLYHFLSCGNKPGHEFLEYQTKYCRTKFPEINITVNQLIAKKNTERYKSCYPLVFISILCSCISFLFLFICLLRSICKKYSCTKQDKSSHNYIPLIPSYTFSLKKHRHPETAVVEDHTTSANSPIVYIPTTEEKKVSCSRRK

SEQ ID NO: 399 - protein, 212 aa, African swine fever virus
MVSRFLIAEYRHLIENPSENFKISVNEKDMTEWDVILRGPPDTFYEGGLFKAKIAFPPEYPYAPPRLTFTSEMWHPNIYSDGKLCISILHGDNAEEQGMTWSPAQKIDTILLSVISLLNEPNPDSPANVDAAKSYRKLLYKEDLESYPMEVKRTVKKSLDECSPEDIEYFKNAASNVPPIPSDAYEDECEEMEDDTYILTYDDEDEEEEDDE

SEQ ID NO: 400 - protein, 66 aa, African swine fever virus
MWKVNDQGFLNISVTGTKFNLIAITGKLGFYTDPPSHLIIMPLKFFPVHKFSKNEPNKKQKRFIYF

SEQ ID NO: 401 - protein, 27 aa, African swine fever virus
MYEIILAIIIILLTIIIFIFIKHPLNV

SEQ ID NO: 402 - protein, 202 aa, African swine fever virus
MLFRYLVWLFRFIEVKNVVSISLLVIGSNYLTTAISNNTSTTISPTTTSSNYLMTAISNNTSTTISPTTTSSNYLMTAIPNIISDKEDDIHFSTDKTVFDRLSPITLYRAIRSTLNDTSTKTMTDHILTRPYRPTTVIFHSDTPQPVKNATQGNIVKKIYRQVLTFFIQPNPLFPCFKNHEVFLNLANILNTILCIILIKNV

SEQ ID NO: 403 - protein, 238 aa, African swine fever virus
MARGQNIRKRTFSDMDTPSDKNIGIHTNSLPKNNLYRRILFKGKISNYSISKDSLAKDHSSNHSISKNGLIGKKRPAPLDISFQNMNSSISSSTQKKTRILDEEIKDQSLSNENDRDSPVIVDITLKPSYMSKTSRITEIIHKMKELNMNRIEDGSSFNKKRSEHDDKNILLHTMEMEEEDCEIEEDIAIDSPYLNTSLSEDDTDSIVGTDYSEEEKETISETESSSDDESYSLYDSF

SEQ ID NO: 404 - protein, 309 aa, African swine fever virus
MLSLQTIAKMAVATNTYSKYHYPILKVFGLWWKNNTLNGPIKICNHCNNIMVGEYPMCYNHGMSLDIALIRAVKERNISLVQLFTEWGGNIDYGALCANTPSMQRLCKSLGAKPPKGRMYMDALIHLSDTLNDNDLIRGYEIFDDNSVLDCVNLIRLKIMLTLKARIPLMEQLDQIALKQLLQRYWYAMAVQHNLTIAIHYFDNHIPNIKPFSLRCALYFNDPFKIHDACRTVNMDPNEMMNIACQQDLNFQSIYYCYLLGADINQAMLMSLKYGHLSNMWFCIDLGADAFKEAGALAEKKIKECYNTY

SEQ ID NO: 405 - protein, 48 aa, African swine fever virus
VLQHILGLNIFKRELIPPCKDPDPYQIQILLKNYILKNVSTVFTYYCQ

SEQ ID NO: 406 - protein, 542 aa, African swine fever virus
MFSLQKKALQHIYMTPENASQLTKDLLQHLGLYWNGPIVKMDTVIHLHNNIFSNRSVLKYALAKQANISIIETLVLWVEPEYALAQALKHNRKDVLECIFSYHLTTPKYHHIMHLTSSQELFEFFHLFICKSKNYNARMECLLYAATLYNFPNILEKNREYIIRHSIGNSLFAIACKERHIHLIAWFVTAGVLDTYDDSTLFNTAFRLGDYSLLEVACDLPIIYPDYLIISMMQTAIQKNYFRFFKKLLTHFNIYRPIIITDAAYYNRRKILLLLLNQNVFNNFAILCALSAAIKGHASKKTLNLLISQLDSQMTVIDSVYYSIIKYNNIDCILLLMQIKTFRLETLVSIAVHGDNIDIIAACKAFLPKDTLYHLVLKMAIISRNHKLFKLYTEKENPMYIFTTMKAIISNLVNYTVVQAVAIEYLRKFHQEKQLPIVPLLMVLAEHNYITKFKKACYAANMSDQKVKRALIKCLFIASQKNYCQIFKYCFGSLLKVLSKHERVKFFNSVVFAKKLASYYDHQNMIHLIDSLIERFRYLIKD

SEQ ID NO: 407 - protein, 141 aa, African swine fever virus
MGNKESKYLEMCSEEAWLNIPNIFKCIFIRKLFYNKWLKYQEKKLKKSLKLLSFYHPKKDFVGIRDMLQMAPGGSYFITDNMTEEFLMLVVKHPEDGSAEFTKLCLKGSCIVIDGYYYDNLHIFISETPDIYKYPLIRYDR

SEQ ID NO: 408 - protein, 102 aa, African swine fever virus
MGNHPIKQDMKTCDYYHGEKKLKYMRRMLYNEPLGTYAVSSLFGCDMIVLTWNCTISGRTLHRRIHTRFGQYYHNNCYYTKIDDIIGDYPDTFYRPLYRYKP

SEQ ID NO: 409 - protein, 103 aa, African swine fever virus
MGNRLIKKDLKKCEYYYGEQQNLKQIWRLLFNEPLGTYVVSSFLKKNYVVISFSCPTNTRIMHLRINICYDLYCINGEYYEKIDDFIRLYPHIFYRPLYRYKS

SEQ ID NO: 410 - protein, 45 aa, African swine fever virus
MYFQQTRSILIKNDAVFILNLGLKFKEVFINYNHGNFFSNCLCKK

SEQ ID NO: 411 - protein, 96 aa, African swine fever virus
MGTFSVTASAKSDDAVCKYLEEPIDENYRNILRNEHVKKNLNEALNRHITTYNPVVDWCNNYSTFSSQDFDEYKIYIHSDLMDGRPRPKKTWCVIM

SEQ ID NO: 412 - protein, 170 aa, African swine fever virus
MFYPVVQVLIGIILVIILILGFYHLKHKPPKKKCKTDTDCKDKGHHCVRGTCTDKSCLEAVKQDIKDIKLDPTIRSCDYAPGFYRFNATTADLQSPFGKTRIDLGRIWTTWSKEDECCQSLCLQHKGCIGWEFDEVSLGGTGECYYYLNPHLALKNSNNNTVMGIARNVL

SEQ ID NO: 413 - protein, 93 aa, African swine fever virus
MDFFLLKKIFFFVDNRMLEPMLVMAPIPLVLIFLYSYFKIKLHKLITIALFLGCLFFILRDFCFPPMLWTQLHNITSSINILGNKSFQVQCNK

SEQ ID NO: 414 - protein, 237 aa, African swine fever virus
MLEIVLATLLGDLQRLRVLTPQQRAVAFFRANTKELEDFLCSDGQSEEVLSGPLLNRLLEPSGPLDILTGYHLFRQNPKAGQLRGLEVKMLERLYDANIYNILSRLRPEKVRNKAIELYWVFRAIHICHAPLVLDIVRYEEPDFAELAFICAAYFGEPQVMYLLYKYMPLTRAVLTDAIRISLESNNQVGICYAYLMGGSLKGLVSAPLRKRLRAKLRSQRKKKDVLSPHDFLLLLQ

SEQ ID NO: 415 - protein, 70 aa, African swine fever virus
MGRRRKKRTNDAKHVHFATAVEVWEADDIERKGPWEQVAVDRFRFQRRIASVEELLSAVLLRQKKLLEQQ

SEQ ID NO: 416 - protein, 96 aa, African swine fever virus
MSTHDCSLKEKPVDMNDISEKSVVVDNAPEKPAGANHIPEKSAREMTSSEWIAEYWKGIKRGNDVPCCCPRKMTSADKKFSVFGKGSLMRSIQKNN

SEQ ID NO: 417 - protein, 43 aa, African swine fever virus
MDIWYLKYVIAYILLLTMLVIGLIYRIIVLIYRSIQAQKKCCL

SEQ ID NO: 418 - protein, 43 aa, African swine fever virus
MVAFKNIKKTLSFRQQQIVCRRPQTIFRVFCTIKYFFWSGLLL

SEQ ID NO: 419 - protein, 356 aa, African swine fever virus
MSTPLSLQTLVKKVLATQHISKEHYFILKYCGLWWHEAPITICIDEDSQILIKSASFKEGLSLDIALMKVVQENNHDLIELFTKWGADINSSLVTVNTEYTRNLCQKLGAKEALNERDILQIFYKTRHLKTSSNIILYNELFSNNLLFQNIERLSLIVYRGLKNLSINFILDDISFSEMLTRYWYSMAILYNLTEAIQYFYQRYRHFKDWRLICGLSFNNLSDLHEVYNLEKTDIDIDEMMKLTCSTYDGNYSTIYYCFMLGADINRAMLTSVINFHIGNLFLCIDLGADAFEDSMELAKQKNNNILVEILSFKNYYSSNTSLLSIKTTDPEKINALLDEEKYESKNMLMYEELSH

SEQ ID NO: 420 - protein, 64 aa, African swine fever virus
MIKKKNYFFGQHLFVRIVDKSAGNNNIYNFFLGEKNNYVTSYIPIGDLSPSESSVIKNNFVSMR

SEQ ID NO: 421 - protein, 48 aa, African swine fever virus
MNIYLVWFLYILLGNLILAVIYCVIDEVVCDNIHIKKNVAAPEMPRRF

SEQ ID NO: 422 - protein, 54 aa, African swine fever virus
MSLWPPQKKVFTVGFITGGVTPVMVNFVWPAAQPQKKINYNRKKKYFRPRRFKK

SEQ ID NO: 423 - DNA, 32767 bp, African swine fever virus
  source 1..32767: mol_type = genomic DNA
gaatataccatattattgctattgccatcaatgagaatgccacgtaggcataggtcatcctatggccggaccaatccatggctgcacttaaaaatatcaaaaaagtttaagttttgggccggcgttaaaatttaaaccttttctggttgatctttagccatgtatagctgcgatgtttggtgccttatctacatgctattggcattcctgatattcgcactaaagtgctatgttacaaccgtcttatgcgtgatttttatccaccttattggccgaagggccgccttgtatttcctgttaggtggtttggccgtattctactggtggcaagcagctatcaataaaatttaatggctctcacttaagatccttgctgtaagcgggcgtttacatactttgatcaagaaaaaaattatttttggacccccccccagttttatcaaaaatcatataataaagtggcgacaatcaacatattaatcaaccacagcattttatgatgtgttaatcaacatataccatattaatcaaccacagcattttatgatgcgtcaatcaacatattattacggagagcgtcaatcaatataatattgagaacagcgacttgataccgtgtatggtggtggcggcggcatgttgtttgtaacagcatttttcatcattcgaagcttacaaaagatatgtataagatagcatattaatgttattaacagtaatatcaataaggcgtagctatagatcttcactttggtagaccaataatccatggttgcgcttaaaaataccaaaaaaaacattaagttttggagggtaagattggtttttcaccattggtaaagattattattctaaatgtttaccccatagatgtgaaacaatgattcttcatatattaacatattttttgacttatacttttcttcatctagtaaggcgttaattttttccggatctgtcgtttttattgataaaagagaagagtctggactgtaatttttaaataataagatatttattaatatccaattattcgtttggctcgctatttccatgctctcttcgaaagcatcagctcctaaatctatacaaaggaataagttaccttcacaaaaattcattaccgaggtaatcattgcccgattaatgtcagcccccaacataaaacaataatatatagttgtataattacaatcatacatacaggccaactgcatcatttcatcaatgtctatatttgtcttctctttgttataaatttcatgaaggtcaaagacgttgttataagcaaccccacatattaaccgccaatctttaaaatgactatatcgttgataaaaatattggatggcttcagtaagcttatatagtatcgccatactataccaatacctagttagcatttcgttgaatgaaatattatccaatgtaaagttaattgataatgtatctagttcaccaaaaattcttaatttcagttgagcattatttaggaaaaggggattatcagataataattcatggcatagaataatattactgctagttttaacatactgtacattataaaatatttctaaaattttattttcactcaaagctttttcctcgcacctaactttggcataggtcctggtgcactccatattgacagtaaccaacccaaagctgatgtctgcaccccattcggtaaacagctctattaaaccatgattgttttcctgtacagccttcattaatgcaacatttaatgttaaaccatgtttaaaacttgctgtttttattaatatttgttcatctatacaagtatgataaatcgtaattggggcttcatgccaccacaaaccacaacgctctaaaatacaataatcatcttttaacacaggctgtgtagctagtacttttttagtaagtgcttgtaaagtagatggcatcttctatctgcaaaataattatttccgaaaaaaaaatcaaattaaaatactaaattctatttttttttttaataaagcctgtaaattatataataaatctcgcccaccgtattatttccggacacaactttttatacctcattatatttttagatctatagttttttaacaaggcattaattttttctggatctgtcgtttttaaagataaaagagagacgtttgaactataataatctttaaatgataatatttctactaatatatcatgattcttttgttttgctaattctaagctctcttcgaaagcattagctcctaaatctatacaaaagaacaagttattcatataaaagttttttaccgaggtaaccattgcccgattgatgtcagcccccaatacaaaacaatagtaaatggttaaaaaattgctatctctcatacaggccagatatatcatttcatcaatattcatatcaaccttttttatatgatacatttcatgaagatcagacacgttattaaaagaaagcccacatattagccgccaatctttaaaatgactatatcgttgataaaaatattggatggcttcagtaagcttacatagtatcgctatactataccaatatctagttagcatttcgttgaatgttatttcattcaatataaagttgatcgatatcttctctagaaaacaacaaattattacttttaattcctctatattctggaaaaggggattattagataacaatttatggcataaaataatattactactagttttaatacgatgtattttataaaatatttgtacaatatccatttcattcaaaatttttgcgcctaactcccggcagaaattccaagtatgctccgtattgacagtgactaagctagagttgatgtctgcaccccattcagtaaacaactctattagatcatagttgttttcctgcacagttttcattaatgcgagatttaactctaaaccatctttaaaaattgctgattttatcatcaattgattatcctcattagtagaaagcataattggagctccatgccaccacaaaccacaatatttcaaaataaagtagtgttctttagatatgtgctgtgtggccagtatttttttagcaagagcctgcagagaaattggagtagacatatttttttttgcaaaatggtttaagtttttcaagaatacagattggataaattaggttgttgacttagttacaggaggtattaaatattatgtagacataaaaatgagatcctccaaaaaaataaacaacaaaaaaaatatgtttaatattaaaatgacaatttctacattgcttattgctcttattatactacttattattattttagtagtgtttttatactataagaaacaacaaccaccgaaaaaggtctgtaaagtagataaagattgtggtagtggagagcattgtgttcgtggatcatgtagctcattgagctgcttagatgccgtaaaaatggacaaacgaaatattaagatagattctaagatttcctcatgcgaattcactcccaatttttaccgttttacggatactgctgctgatgagcagcaagaatttggaaaaacacggcatcctataaaaataactccatctccaagtgaatcccatagcccccaagaggtgtgtgaaaaatattgttcatggggaaccgatgactgtacaggttgggaatatgttggtgatgaaaaggagggaacatgttatgtatataataatccacatcacccggttcttaaatatggtaaggatcacatcatagccttacctagaaatcataaacatgcataaataaatacattaggctcatcgtatctttttaaaatccataaatattcgtttgatatatgctgaaatttttataaaaaaaataactatttcctataaatcatctagaaatagtcctcgttttgatcggtttatatcttataatattgtgcatcgatgcacaactgctttttttggtccttctggaacatcattatattttctttcattaatataccattcagatgtaaacgttgaataatttttatggcaacaatctaccattgaattatatttagtaacatctaatacatcgtttgttttatcaggctcagctctataatcttgataatttttgttatcagcttctaaagctccatcattatttttcaaagaagtatccataattatgtttggtaaaaatactttaagttttaatgtgatatttaaaatggttgttatataaatttaccgcttacaggtaatctttattcagtgtcataaactatacttttgatgattcagtattttgtgaatcagtacatttattatcattaatatttttaggctgtttttccaatgttttattgttgcaatgagcctgctcctcctttgacgaggaagtgtctgttggagtcatctgtttaggaagagtatcatccatatctattatgaagaaaatatataaatattgatatacaatcaaaaatatttttgatcacgtctttgttatctatcgatattgttgataacgtcttgaataacctacatcatttttttacataaaaaaatagatataatttttattatatctcaattattttaaagataattatcaatacagcaaatatcataagctaacatatttttcgaataatagttttttagtaaagtattaatcttttcaggattggtttcttttgataataagataggattcgctttataaatttttaaagataatatattcacaatgatagaataaccgtatatatctgctaatgtcttactgtgttcaataacattagcccctaaatccatacaaaagaacatattttcaatacaaaagttttttaccgagattaacattgctcgattagcgttggctcccaatgcaaaacagtagtaaatggtcaaaaaattattatcgcgcatacaggccagctccatcattttattaatactcatatgaattttcgttgtgttacatatttcatgaaggtcaaacacattgttgaaagaaagtgcacaaattaatcgccattcatcaaaatgcctgtattcttgacaaaaatattgaatagcttctttaagattatattttaccgctatgccataccaatatttggttagcatctcactaaatgagatctcatttaacatagaatttgttgttaaatccttcaactcccaataaatgatcatccttaaatccaccatgtttacattttgtaaaaaagggttattagaaaataattcatgacacaaaatgacattactacttgttattttacactttgtttcaaagaaaaatcgtaaaatttcacttgtctcaagctcttctttagctcccaattttcggcataggtttcgagtatgctcgttattaataaaaagtaacccataattaatatttgcaccccattcagtaaacaacatgattagatcatcattgttttccttaactgccaataccaatgcagtattaagccttataccctctttaaagcataatgtccttatcattatttgattatcatcatctatatacattgagataggagcttcatgccaccataaaccataacgctctaaaatataataatcatctttagatacgtgttgcgtggccaatgcccttttagcaagtgcttgtaaagtcgatggctgcatgtttattctgttaaaaaaaatcaaattatcgggtaaacataaggatcaacccgtagttaatatttgcagtagtattttttaacaatgaattataataaaaaaataattcattactatctattataaaacccatctttaactttaaagaagaactagatcatcttttttttgttgtgtcagaacttcttcaatttattacccacattttatctaaaaaaataaaaactacatcatatcttgtttcttcatcaaattatcataccatttatagggtgtaggttgggaacattccatcatgtggtaatcagggtatttatatattttttgatagtaacatctatttggcagatgtattgtccaacaatcatgtctaataaaatcattttcacctatgggggaatcatcttaaaaaccttattcctacagattccattttgacagtcccagcaaaagtcacaatattttccatgagtacaccaatgttcaagctctctttcgggaggaatgctgccaattttatgttttttagcttctaactctctgtacaacatcagttgggaaagcagaaagaagattaccaggagaaccattaaatatataatagtctgcaaactacgtttgcgaatgtaatttgcaactaaaacacaacccacaaggtaaaatccataagttaataacttttgccattttcgtatgacagcctcgtgccattcatggttgtgttgtgggcattctgttcggtaaacttcatgaggctttatagaagttacatagtaggtacagaattcattgtgacgaaaaacactgcagttagctatgtagtcattttcaagaatgggagaatggttttcaaagaccttattcttacagatgccatcttgacagtcccaacagaacctacaatgatttgcataggtgcaccagtattcaagctccttttcaggaggggttcttgttagatccaggagctctagctcatatgtataaagaagagttggaatggatagtaaagtaaatatttgcagaccaagcatggctacttgtgaacaagtggctgctcgtcaacaaatagctgtttatcagcaaatagctgtttatcagcaacaactaattatcagcaaatgctgcttgtgggtaagccaataaataggccatacccttgaaaggagaattcagtttgataaaaaaaataacgagttttctaataacccggtcaagcatttaataaatgaatagcatcacacgtctgcatcgtgcattctgcctggaaaatgggcccatctctaatatatttacactgacggtgaatcatacagtgttccatgggatagctatgctcctgtacaggaggcatatcttttagaactttattcttacaaagaccatcttgacaagcccagcaaaaccgacaatttttcacatattgacaccagtatctaagctcctcttccaggggattgtcggtcgaaaacccctgtagactagctaggccagctagcagcaagccgaggtaactaaagaacctcattgtagtgttatattacgaaaaaacatgttaaaatttggaaaaaaaagccctttttatagatctggaaaaaaattttcacaaatctaattaaaagccttacagatcatccttttcataaattttcattaacaattggtgggggcggttgtgaggtactggatcagaacaatccataacatggtaatgtccatttccttcaccatatgtacactggttataccagcgagaaacctcacaagatgtcaaataactgttctcaacaatcaatggcatgctcttattcaccttgttcttgcaaattccatgtgcacattcccagcaaaacttgcagttttccatgtaagtacaccagtatccaagttcttcttgtggaggattatccgttgaacgaagatgccctcctgcctgagtaggtagtcctaagacctgattggccagcaggccaagaatttccaagaagatcaccaacattgctacggctggctgaacagctggcagatagctagctaattagcaaaccaagtgactcgccctctctactcttaatatgagaatttaagattcggtccggcttttttcccatgttttacagggaaaaggtatttttagcctatgaatgtacatggttccgcacattaaaaaaaataaaagaaattatttaatattggctgttattttctttcaactagcaacaagccaggtaactaaagaacttcattgtagttttatattacggaaaaggttaaattttggacaaaaaaatcatatctaattaaaaatcctcacagatctttcttttcataaattttcattaacaattggtaggggcggttgtgaggtactggatcagaacaatccataacatggtaatgcccatttccttcaccatatgtacactggttataccagcgagaaacctcacatgttgtcaagtagctgttttcaataatcaatggcatgctattattcaccttgttcttgcaaattccatgtgcacattcccagcaaaacttgcacctttccatgtaagtgcaccagtatccaagttcttcttgtggaggattatccgttgaacgaagatgccctcctgcctgagtaggtagtcctacgacctgattggccagcaggccaagaattcccaagaagactaccaacattgctacggctggctgaacagctggcagatagctagctaattagcaaaccaagtgactcaccctctctactcttaatatgagaatttaagatccggtccgacatttttccgatattttacaagaaaaagatatttttagctacaaatacacttcatatatccctaaaaaaacaaaaatttatttaattttaactattattttctttccactctctctttaagattttgtaaggattccagggctttggttcagaacaggccattacatggtgaatcccctgtcctagatcatacatacatttatttagccagcgggaaactatacatgattgcacatactcattttcaagaattgttgtattctccaatttgccctcacaaaggccattttgacaattccagcaaaacttgcagttttctgtataagtgcaccagtattcaagttcttcttgtggaggattatccgttggatgaagttgtccagctggttgattaggtagccctaagacctggttgcaattcatggtatggtagatacccttatctaaatcatacatacatttatccagccaacgggaaaccagacatgatttcacatactcattcttgtaaattactgacccatctattttgtttatacaagtgccgtcttggcagtcccagcaaaattggcaactttccatgtaggcacaccagtattcgagttcttcctctggaggctcctctgttggacgaagttgtccaacgagctgacttgaaacctggctggccagaaggccaagaattcccaagaagatcaccaacattgctacggctggctgaacagctgactgaatagctagccaattagcaatccactgtacttttcataagatcatttaagattcggtcggcattttttcaatagtttgctaggaaaaaatttttaattttatagattcacactacttcattctcatgcttaggaaaaaaacaaactaaatcttacaatgtatctggatctaatgagaagctagaattcatcttttttcaaatcctttctgggatgttcattctttttccactccttccttgcaattttataaggattccagggctttgggtcagaacagttcatgctatggtaaatgtgctcctccacatcatatctacataggtcaccccagcgggaaacctcacaatattttacatagtcattctcaataatacttgtggagttgtttccccaaaccctgctggtacaaatcccatcttcacaatcccagcagaaccgacagctttccacataagtgcaccagtatccaagttcattctctgggggttcaaatgttagaggaagatgtccacctacccgagtagaagtggaggatgaaaccaggttgctactggccagcaggccaataattcccaggataatcaccagcattgtgctcaaccagcaacggctagcaacgactagcaactgactagcaatagctagaaatggctagcaatcagtagtagctaacgctctactctttataagaaaatttaaaattcgatcagatttttttagaattgagaatgagtaaaacgcttatattctttttctagctagaaaaaataagctagtttaagataggatttcccttactaacggtttaatttttagcaaaggtataggtaaaatacacttgtacttagctgcaaaaaaataagcttatggcgtataagccgccataagtttatttaattaaaatgttaaactctgtgataagactggaatcttaggcaggtttgatgtggagaacagcatgaaatacaagagtgcctgttacacgaataagttctctcaaaccggggatggtcatactcacatctatgaaatcctggtctaggagattcatttgatgcatgatggccgcacccacacttatgagacactgaagaactaaagggtttaattttgatctgaatggtactatataggatgatggcaatccatatcaagattagagcaatcaaaatcacctcctcaagaagcatgatgtagccttaaatcttagactgctttaaaccttaggccctcactatctttaatgaaggagtttaaattttgatccctttttcaagacccatttagaagaaaaaataaagtttatatcaatctaattcataagtcatctctttcataaatcttcatgtattctctatgtggataagtatgggatgttggatttgcgcagtccatttgatgatctgtatggtttttgggtccttcataataactacatataccattccagcgggaaaccgtgcaatttataatccagtcattttgatgaataactggccaatctgtttgaatcctgtttcggcagataccgtggacgcattcccagcaaaagtcacattggtttgcgtaagtgcaccaataaactagctcatgttcaggaggataacgggttggtagtaaatcttctaatttacgtataggagcggcttgaaggacaaccacccccagtagtactagaatcagtacctttatagtggccaccctacactagacctctaagttgaagacaaagaactaaaatttagagccgtttaattactactaataattatattttttattgtctacaataggattctattaaaaaataatgatttttaccaagaaatatttttataaaaaattaatatattttgtaataaactttatttccaatgactgttaaaataaggaaactatccttagttagtcgaggaagatggttaggttatttcgcaatccgataaaatgtttattttatcgtaggtctcgtaaaatccaggaaaaaaaattacggaagagtttaaaaaagctaaatttttaccaccctccagaagattgttgtcaaatatatcgtttgctagaaaatgttcctggaggaacttactttattacagaaaatatgacgaatgatttaattatggtcgtaaaggattcggtggataaaaaaattaaaagcattaaattatatcttcatggaagttatattaagattcatcagcactattatattaatatttatatgtatcttatgagatatacccaaatttataaatatcccttaatttgttttaacaaatattataacatctaagtaaatattcttggaatggattttcttatagaatggttacaggatatgtcagcgacaggcttaataacaaatttgttaatatttttttgttaaataaatgaacaggccaccatttaatattacccgttgcaaaataagaaaaaaaaacaaacttatagttacaaatcatcttgattaatcacatgtcgttttaactcaatgaaccattctaaatctttgggttgtgaacaattcatgttatgttgatagtgtatcctaaagtgagcttcatacatacaccggtcatgccaccgggaaactgtacaattaacaatataatcattttgcgtaataatagggtggtcactaaacactttatttttacacattccatctttacaggtccagcagaagtcacagtgttttgcataggtgcaccagaacttgagatccctttcaggaggcctacgcatttgcatcggattatctgtggaaagaggtaggttcattattatgttcgtcatcaaaattcctaaaagaacatagaagccaagaaagataagcagtcttgtagcggcttgcattcgcattcgtgagtattgtttgcgaacatagcttatgagagcaatggtagctatcatacaaagacaagtatgtttgatattctcagtgtcaatgaccctatcctcctttatttgcattaactcatcaaaccaatcataatatgtgggatttgtacagctcatgatgtgaaagcggcgtatcctagagtctgtaaagtagctacatctttcattatagcgagaaaccctacatatttgtatgtaatcattttttttgatgagagggtgtttttcaaaaaccttatttttacaaaccccgtgtcgacaattccagcagaagtcacacgattttgcataggtgcaccaatactcaagctctctctttggaggtctccgggtcattggtaactctcctgttcctggaaaagattggctttgaatgaccggctgcatgaccgccagtaccaaaaggaacacaatcaccttcatggctgcaacttataagttgcaacttatgggttgcaatactgcaacgtataggttgcaccttatagatcgcgactcaaaaggtatgaaaaccttaccctcaatacagaatttaagttttaatcctgataatgtatctgtttatgaaaaaaaattttttttactcatgtatgaattcttatacgaatcataatatgtaggctgagaataataattcatatacggtgttgcgggctcaataaaaattttgttaccacaaaaaataaatgctggatttttaagatatatatctattaatgactaaaccctttatacgctgtaggctgaaaacaatccatataatgaatatacggtgatttgggtttaataaaatacatacaacggtcaaaatagcgggcaatactacattgactaatataatcattttgtttaataagaggcatatcatcccacactttatttttacaaataccgttcctacattcccagcagaaatcacagtgttttccatacgtgcaccagtattcaagctctcttataggaggcgtataagtccttggtaaattttgtttcatataaaagatggaaaggggtcgatttaaacccggctgagatagccaaatcaaaatacataaaagagcaagtagtttcatagtggtatttagatgtaaatttttatagtatgcaaatacaatgtaacctacaaatacaatactaaatacaaggtaaaaacaacaatgtcttataatgattggccaataatcaccccccccccccccatttttccatgaatatttcatttcctgtatagggtctaggatgtgaacactccatgttatgatgattaggcattttaactgatatttcataaaaacacccccaggaattgcgattaactatacagtttacaatcgaattcatcgaattagactcatttgttatcttatttttacaaatgccattttgacaatcccagcagaagtcacaattctttacatacgtacaccaatatggaagctcctccttaggaggatgctgggttcttggtaattctggtaattcatgtgcaagaatgaggactgagtagcccaacaaaagtcctagaaccttcatgttgtgtccaaatggcacctgtcattttaaaaaagatttaaattttgctaccgcaaaaaaaatccagtatgtatttttttaatacatataattattgaagtcttataagataaagccgagaacactatattttgtatagatgatgtatccggtattcaaactctcttataagtacatgtaggaaatggtcaattattcaagattggctgagataacaacaaaaccaaaatactcaaaagcataagtaatttcatggttgtactcagtcgtagatttttgcagatcgcaaatgcaacgcaaccagcaaatacaaagctaaatacaaggtaaaaacaataataccttataatgattggccaattcttatccctccatttttccatgaacatttcatgttcataaagtctaggatacgaacaacatttcatgctatgatgattaggtattttaagtgatatttcataaaaacaccacggggttgttggtgattgataggtaagaataaggatggttgaataacctagtaaaagtcctagaaaaaccttcatattgcgttcataccacagatgttatttaaaaaaaatataaattttacagtatgtgatatacacataccacaaaaatgttcttatattaactaaaatatgtgggcagagagcaattcatataatgaatatatggtattttaggctcaataaagtacatacaacgatcaataaaacgggtaatactacatttactgatgtaatcattttgaacaataagaggcatatcatccaaaaccttatttttacaaataccattcttacaatcccagcagaaatcacagtgttttccatacgtacaccaatattcaagttctctcataggaggcgtataggtccttggtaaaatttgtttcgtataaaagatggaaaggggtcgatttaaaactggctgtgctaaccaaaccaaaatactcaaaagaacgaaaagtttcatggttgtactcagacgcagattcttacaaagcgcacatacaaagcagcctgtatatgcaataccaatgatgaaatagagacagtattgctttatagataattgttgatggtcaccccccccccccccccatgtttgcatgaatatttcatttcctgtatagggtctaggatgtaaacattccatgctaaagtgattaggcattttagatgaaatttcatataaacaggattgagtcttggaatcacggaaaactctacagtttacaatagaatgattggagtcaatgaaacgagattccgttatcttatttttgcaaatgccatcttgacagtcccaacagaaatcgcattgtggtacatacgtacaccaatatgaaagctcactcttgggaggatgctgggttcttggtaagtctggtaattcatgtgcgagaatgaggactgagtagcccaacaaaagtcccagaagaaccttcatgttgcgtctaaatgacacctgcacttacaaaaaaaaatttaaattttgaatataacacaaaaaaaccaccttaaaatttcttatattatttcttggatctgccccgacgtcatacaatgtattaaaattatagaccaatcatctttttgtatataggctaatcatctttatatatagattttagatgtttgcttgttgtatcaacttaactgctagcgaagaaaatggataaaaactttctgtatttttataggttgaaatcattttatgcacatcgctaggatctaatattttattttgaagaaccgaatgtgggcttaaaatttttttcttagaaaaaagtagaatcataatattgctatgtttttgtttaatgatttcttgtatcttttttgtatacgggttggcacccaaacctatacaaaaatatacattactcaaataactaccttctatacataatcttttttccccacgtattttcctatttatttccctatttatggaattaaaggatatcaatctctctaaggcacggtcaaggtctgcgcctaaggcaaaacaataatatatacctaatttattcccagggcgtgcacaggcaagaaacatcatgacgtttagccctaaacgtatattttcctgaaaatacgcatgatgaacttcatcaatattacctaagtatatggccgtttgtaaacgccaaagatctaaatgaggaaattttttactaagataatgaataggttttgtgagattaaaatctatggcgaacttataccaaaattttaatacaagtgtatttctcgtcatttcttcttctttttcatctaaatataagataaaacgattgtaaacaaagtctatcaataggtgaaaatcattgctattaaagctgtcgagaatcaaaatattgtcataataaatttcgatcgccagtaaaaccttttttcgtttgacgagataaacaaacatattatacaaccctacatctaaaaattctggattggctcctagttggatacacaggtctttagtctgcttcgttttggcacacatgatgccaaaattaatatcagcaccccataaaacaaataacttgattagatcagtctggttttccttcacagcttttactaaggctctgtcaagctcatagctgtcgacatcagagcatgacatagagccaccggttaccattttacattgcttacaaaaacctatgggtccgttttcccaccatagtccaagctgttgtagaataaaaatatcatcctcatgataatttgaaaaagccttggtttctatcaagactttttttgtaagaacctgtaaagagttcatcgtattattatgaataacaggagtaaacgtaatcaattataaaagtgattttttcgaaaaaaactttagatggttgaaaatgataatgtacatgttcatacaaaaaatagatgcagtgatgtctaaaatcaaaatttaattttctatgtaaaaagtacagacttacttatttgggttaaattgtttattttaaactttaattaaccgtttgagttagcgatgtttgatttatcttccatactcatccggggggggggggtccttatagctctgacattattgtggattattgaatataatgaatacttcatagatgctaaacattttaatagtagttctgaggcttaattgtactctataaatttataaaaactttttgatcaaaatttaatttcttataaaaagagtacagacgtcgcttgtttaagcttcatcatgtttcattcattactttctacaattacggggggggggagtcccctcatagctttagtattgctatggtttactaattattatgtagaatttatagaagcatatgtacctgaaagtatacctactctataaaattaaataatttcagtatattttttttatgaatagaacggaaatgatataaaaataatttaatattgcaaaaaaaattcataatgttggtatgtattataaacataatagcatgtgtaatttataaactgactcctctatataattattagatgaggtaccaacctacttatgatatgccgatgatagatattgtatactataaaacaaaattattttaaatgtattcatggatacattataacatttttaccgcaaattgtctctcagcgaagaaaatgaatgaaacgtttctgtatattcataggttgaaattattttacgcacttcactaggttctaatattttcttatgaagtattgaatgggggcttaaaagtcctttcttaaaaagaagtttcatcataacattcttttcttgtctaagaagagtttcttgtattttttttgtataaggattggcacccaaacttatacaaaaatgtacattactccaaataccataatttgaaaagaaagttatttccctatttacttcatgattaatgaaacctatcaacgtctctaaggccgtattgatatttgcgcctaaggcaaaacaatagtatatacccaatttattttgagggtacatacaagcaagcgacatcatgtcatttggatctaaacgtatattttcctgaaaatatgcatgatggatttcatcaacattacctaagtatacagccgtttttaaacgccaataatctaggtgaggaaatttcttactaagaaaacgaataggttttataagattaaactctatggcgatcttaaaccaaaattttaatacatatgtattttttatcattttttctttttcatctaaatttaagataaaacgattgtaaataaagtctatcaacacgtaaaaatcatggctatcaaaactgtcgagaatcgaaatattgtcataataaatatctatagctaataagaccttttgttgtttaattagatcaacaaacatattatacaaccctacatctaaaaattttggatcagctcctagttgaatacacagaactttcgtcctttccgtcttggcacatatgatgccataattaatgttggcaccccataaaacaaataacttgattagatcagtctggtttttcttcacagccctcaccaaggctctgtcaagctcatagctgtcaacatcagaacatgacatagagccactggttaccattttacattgtttacaaaaacctatgggtccgttttcccaccataatccaagctgctgtaaaataaaaatatcatcctcatgataatttgaaaaagccttgttttctatcaagactttttttgtaagaacctgtaaagaattcatcgtattatcatgaatgaaagcagtaaatgtaatcaattataaaattgacttattgaagagaaatgttaaatgagtgaaatcggtgtttatgatgatgtacatgatcatacgaagaaacacgttcactggtgtccatgatcaaaatttaatgttttacgtaaaaagtacagatgttaactgtttagtttaaacataaatttaacctttagtttaaaccctagttaatgatgtttaatatttcttctatactcattcagggaagtgtaatgattctaatactgttgttatggattattaatgaaaactttacagatgctggagggaataattttaatcatactgttttaatgtagctatataagctttcatcaaaatttaattttttttataaaaatacacgaattaaactaaagtctaaactttagtttgactatttgagttaatgatgcttaacttatcttccatgcttatcaagggggggtcctaatagttttgatactattgttgtggattgttgaatataataaatactttatagatgctgaaatgtttgaaaataatagtacatcaatgttgtaagtttgatcaaaatttaatttctcataaaaaaggtacacatcaacattgctcatttaagtttcatgatgtttgattcattacttcctacaattactgggggggggggggggtctttaatagctttagcattgttatggtttgctgactattatgtagaattcatagaagcacgtttagatagtaatatcactgcagtgtagattatgaaatacatactaaactaatttcagtatattttttttgttcatataagttaaggtacaaaaatgattaaacattgcaaaaaaagaaaatcacaatgctattatacatagtgatcatagtggcttgtatcatttctaaactagttccaaatgaatattgggcaatacatctattttttatcattatgatttttatggtatatatgtatgaaaagttagatatacatcaaaaatctcagttctggaattataccatgtcaggcttatctggacataacgtacaggtaacatgtaagtgttactaaatactatgaagtatctattttttttgttgtaaaaaaaagaacttgatagtattttttaaaaaataaaataattaattgtacgtcaacttccttattttattctttaaaaataactcgtaagtattatttatctattttttgaaaaaatagatgtaatcggtttcatcatttaggtgtgtatttctttttagcatctatcaagaattcattgtttagtgatatgaaaacaatgaatgatcattatcttctatttaacaaccacctaaataaatgaacgtctttttcatcttaactgattaccaaaagttattttgcgaaaaggcatacatatgatcaatatcagacctacaatgaatatttccataatatccctttattgtaataattctatttttgcattccgatatctcatcatctgtgctattatatgtttccataactgtttcatcatcaaacataaatcctgttaaataggcaaaagactttaatcccggatagatttttaccattttcctgagagccgtgtatagcttgtaataaatggccaaaaatatgcaataaagcgtagaaagagagtaatttttggcataaaagattttgaaggtttgatgaatggctaaatcgcatataatataagatacgattttaaagcgcacctgttcacgcagatttgttgaaaaattcgtggaaagatttaacaaataaaaggttattaatagttgctcatcattccccttatacgacatcgtcagacgctctaatattttactactaggcacatctgccacatgttgaacatttaaagcctgttcttcttctgtgttacggcaaaagagccgtgcgtattcaggtgaagctccccaggataacaacgtccttgctacggctaaattttttttgacgatgacttttatcagaaataagtctttatttttgcattgatcactatgcgaatttgtatagttgacgccgttgcattgagtacattgatataatgttttacaattccagcgtagccctaaatggtataaaagaactgtattttcgacataagcatgctgattaacgatgtttttgagacaacacgtcgttaaggacaccatattgtctccaatttgttagataaaagtctttactaaaaaaatagatttttagttttaacaatcgagattttattatttggatgcatcatcaaaaagatttataagtataagaggttgtataagaaaaaaatgatgttatactatttatgttaaaatttaatttatcatataaaaagtacagatttaatcagttggttaaactatttagttaattaaactaaatagtttaaccatttagtcagactacttggttagcaatgtttgagctttcttccattcttatccgggggggggggtcctaatcgttctaatactattgtggatagttgaatataatgaagactttatagatgctataatgatgaattctagtatgcctgtataaaataattaacctttttgatcaaaatttaatttttttataaaaagctacagagtagtgttttattaaacgtggcttatttaaaagttacacaatgttaaaatctctacttactttaattctttgtggggttttattaactttatccatattatggcttactacttaccatgtagaacttatagaggcaatagatgatttctacgactgaaatatagaatagtccattttctatttgtaaaataatgatttatattctttcctaaaaatgatactttatatggtttgaaaacaaatattaacaacttgatttttttttctataaataaactataaatgaaaatagtaaaactcatagagtcttataagtgaacatcttcataatgttactcaaacgttggactattaaaaaatattccgtgtgcattattgcttttaatcagtatgattactttatacgaagccgctattaaaacgcttatcacacaccgaaaacaaattttaaaacaccccgatagccgtgaaattttactagctttggggttgtactgggataaaactcatattcttgttaaatgtcgtgaatgtgggaatatgagtcttaccggaaaacacagtacaaaatgtattaacattaattgtctacttattcttgccataaaaaaaagaataagcgtattgttgataccttgataggaatgggcgcggatgtaacatatatacatcttttaaagaataagataaaactgtcatacaaccagctgtctatgcttaaaagcaactcgcagatttcattgaaggagcttcatgctatatgctatcttttatatggtcggcttcccaaaaaaattaaacaagggatgcgactgtgtaaaacaatggcgggactatgtggtgaacttttatgtgcatttttagctccgtaaatgataatatgtatttaaaacaaacagatattaccaaaatatattctatgtacataatatctgggaaattatttttttttctcatacccttaaatataaaaatattgggtttcttcactaaactttagaggtaaaaatttttctttgttttgcaccatcatgtatgggtttaggctgtcccagggattgtttatttgaatatttcctaaataggaacacaacgccatgatcatatatctttcattctggtaagctttttgatacatcttcaaagatgccgtacctccgagtgtgtaacagcaaacaaacgtccgtacttttccatgggtcgcagcccattccattccgtagctcagcatcttttgctgtatttttttattcgctttataaaaaaagtttttcatccattccacgttctcataaaaacaggcacttaaaaagagcactaggggtagtgtagtcttattatagaatgtaggaatgtatgttttagttatttttttcaacgcgtgttccatactatgttttaccgccataaaaatacaaaaccaataccaactttttctataaaaggttttgctgtacacatataaacgagcaaaatatatttcaaactctatattctttttataaaaaaactcgagacagtcgtttatgttacgactttttctaaatacctcaaaaacagtaattaattcactgtcgctgtggaaatgttcgtaagctaactgtttaatgtctttaggggtcaattctttttttgggagcagtggtttgagattcggcaaaggtcgtctaaagtagtgagcgaacttttcattcgctccccaacacaaaagccgataagccagcatgtagttatcacgttttaccgcgtaaataagcaaatagtttatattgatacatgtaccatgttgctgcccgtttggacatatgttgccgcattctgaacacttatgaatgagatcatagttcttacaacataaccccaaacgggttagtacttctttgtcacgttttaaaaactcgacatgattctttaatgttaatgctttgagcgcaatgttaaataaactctgcattttattaaaatgaggttagtatcatgttttagtataaaatttagcggctgtttacataatgctaaataaacttaacgttcctactaaaccaaaaaaaatcaaattgactaagtcatagagaatttgacgatgttggtaggtaattttttaacatggtatatatttttttagggtcggttatattaggtaataaaagaggacgtgccgttaaagtattttgcttaagatcctttagatccttacaaaaatatagattgttcgtctgatgatgccactgtgttgcagtgatggcttgatcaatatcacctcccaagacaaaacagtagtatatcgttaaaaagttgtaatctttcatacaagccaactgcatcattttatcgatgtccatatgaacgatcttttgctcgtatatttcatgaaggtcaaatacattgttgaagtaaatggcgcacatgagtcgccacatactaaggtgcccatatgtttgatagaaaaaggagatagctcttttaagcttatattttactgctatggcatagcagtatttaacgaatacgttcatgggtacattatctaagatataaaatatgaaaaactttaactctcgatgaatctcttcccccatttcctgtacatttagagcttccaacataggatttttatcaaatatttcatgacataaaataatgttattgctcgttttatgacgcattaaaccggtgaaaatttccttattatttaaactatctttagctcctaactttcgacacagctcctgagtttgttccgtcctagcacaggtcagcccataataaatgtttgctccccactcggtgaacagccttattacgtcatagttattttcttttatggccatgattaatgccacatcaagatgaagaagttcccccttaaagggggttgagcttaaaataacgtaattacagtagtgacataagctaatgggcttgttttgccaccataagccacaatattttaaaatataatgatactcctcaggcacgctctgtttggccacagcctttttggccagggtttgcaaggagagcatgataacttcttgaaaaaaaaactcaaattaagttcctacttttttaaaatattagtatggacagatctaccatcatatgaaggaattctttcatcgttaaacactgaagagataatactttcatcgtatagagaatatcatgtcaatccatatattgaatgttatatatcattaaacccatcattaatatagtgtttatgtgctatggacaggttttttgaatgataatcttttaacatacgttttataacttcgggatcagtttcttttaaagataaagaatcattcatgttataacaatttaatgataacatgctggcaatgaacgagttgtctttttgatgcgctagagtctttccctcctcaaaggcattggcgcctaagtctatacaaaagaatatgtttccgatattatagaactgaatagaatgaaacatggcctgattgatatcagcccctaagacgacgcaacagtaataaatcgttaaatagttatagttcttgcgacaggcccactttagcatttcattcatgtctatgcgaatcctctccttttcgtacacttcgtgaagttcaaacacattattgtaaaaaagggcgcacataagccgccaccgatgtagatgagcatatctctgataaaaatagcaaatcgcctccttaaggttacattctattgccatcgcgtaccaatatttagtaaacatctcgcttaatatatcggtttctaccattaatccctccagttgttcataaatcattccctttacttcaaaacgatttatggtatctaaaatgggattattagaaaatacctcatggcagaaaatgatgttactgctagttagatcacgtttcaatgtgtaaaaaaatcgtaaaatttcctggtcatttaactgttctttggcacctagctgcctgcacaggtctcgggtgtgctccgtgttgacagaaagcaaaccgtagttgatgtttgcaccccactcggtgaacaattctattagatcgtgattgttttcctccacagctttcaccaaggccgcgttaagatttgtgccgttcttaaaatacggcgtccatattttcttttgatgatacatgatagggccattatgccaccatagaccgcagcacttcaaaaaatgaggatggcatttggccggatactggctggccagcacctttttggtgagagtctgcagagagaggaccatatttcttttttttgaaaaaatcaaattaaaaaaatcatgcttgtttagcatacatgtaatattgttataattacgttataattacgttataattacgttataactatattataacaatggtataacaatggtataacaatgttataacaatgttataacgatgtatcattgatgtcatcattcaactaggccaacatactttttaatttatagttttttaatagatgatatattttgttaggatctgcttcttttaacgttaatagcgaggagtctgcactataaatgtctaatgataaatgatgagatatcaaatagtaattccgttgctctgctagggcctttgcctcttcaaaggcgtcggctcccagatctatacaaaagaacaagttatccatattataaaatcgtacgcaggcaagcatagctgaattaatattagctcctaagagaaaacaataatatatggttaaaaaattgttatcttttgtgcaggccatccgcatcatttcatccacgtccatgcggatcttttccttttcatacaaattatgtaggtcaaacagcttattaaaacaaagagcacagattaaccaccacgtatttagatacttaaaatgttggtaaacataagaaatggcctccctaagattatcctgcaatgccactataaaacagtatatcgttaacatatcaccatccgacatattacttaatatgtcggtgtcttctactaaccttttcaacttccaatatatggatgaccttatttcccttataatgacataggctggaaagggattatcattaaaaagtttaagacataagataatattactgctagtagtgccagggtgtattaatttaaagaacatgtgcataatcttctttttatccacgcggtacttggctcctaattcccagcaaaattctcgaacaggcggcgtattggcgcaaattaacccatagttgatgtctgcgccccattctgtaaacagttttattaactgatagttgttttcctttgtagccaacattagtgccgtattaaggtccaagccgtctgcaaagcttggcagctttatcagcatatgtttgcaatcaagggaaattggggccttataccaccatagtccgcagcgttctaagataacatggtactcaatagatacttgctgtctggctagtacctttttggcgaaggattgtaaggaaggaaacatcctgtttctttttttttaaaaatcaattatctttgttcataatcaagaaaaatccccatatttattgagtgataattttttaacatgcaatttattttttcagggtccgtaacgatcgacaacagagaaataaccggattgtaatgctttaatgataaggcatgggctatcagataattttccttttgttctgccaaagctttgccctcctcaaaggcatcggcacccaggtctatacaaaagaacaggtttccaagattatagttttgtatggaaacaagcatggcttgattgatgttggctcccatgataaaacagtagtaaatggccgaatagctataatcttggatgcaggctatgtgcatcatttcatcaatatccatgcggaccctttctatttcgtacagctcgtgaaggtcgaacacgttgttgtaaaaaagggcgcacatgagccgccacctatgtagacgcgggtatttctggtaaaagtagcggatagcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgctactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaaacaggttgaccttataaataatttattgcgtcgaatatgaagcataatggggccattatgccaccacaggccacaacacttcaggacatgatattgatctaccggtatacactgcccggccagtactttcttcgtgagggattgcagggaaggcaacatgcctttccatcctttgacggaaatcaaattatctactaataactatcagtgtttatattaagtatttagatattatcccgggctggatacgtagtatcgctattcacatgtacttccaactctagccggagcctgcagggtcatttatttttaatattgattcttttttgtatttaatcatttagagaaggtcatcataggagccagatgttctctctccagaacttatgtcgaaaaacattacctaaccgtaaacttcctgaattttttgacgaatatatattacaactgctgggattatactgggaaaaccatggaactattcaacgagcaggaaacaactgtgtgcttatacagcaacataccctcattcccgtaaatgaagccctgagaacagcagcatctgaagaaaattatgagatcgtgagccttttattagcgtgggaggggaacctttactatgctattataggggctctagagggcaaccgccacgacttaattcgtaaatatgatgaccaaatcaaggaccatcatgaaattctgccattcattgacgatccagtcatatttcacaaatgccatatcatgcggcaatgcttttttgattgtattttatatcaagctgtaaaatatagtaagtttcgcgttcttctttactttaaacatagattagaggatgatttgcccttcactcatttacttattgaaaaggcatgtaaagatcataattatgaagttattaaatggatatatgaaaacctacatatctacaatatgatagatacctttgaatgtgctattgcccataaggatctacatctatattgtttggggtatagatttatatataacagaatcgtacccgataagtatcatcatttagatattcgcatgctttcaagcctacaactcctacataaggtggcagccaaaggatacttagattttatcctagaaaccttaaagtatgatcataataaagataatataaatattattctaacacaagctgcaacctataaccatagaaaaattttaatctatttcattcctcaatcaacccacgcacagatagaacaatgtttactagtggcgataaaagcaaaatcttccaggaaaaccttgaacttactactgtctcacctaaacctttccatcaacctcatcaaaaaaataagccattatgttgccacttacaattcaacaaatataataggcattctgagtatgcggcggaaaaagaagatatatttagatatcatattgacaaaatttgtaaaaaaagctatttttaataagtttgtcgttcgatgtatggatacattttctataaacccggaaagaatccttaaaatagccgcgcgaataaataggatgatgttagtgaaaaaaatatctgaacatgtttggaaaaatcatgcggttagacttaaataccttaaacatgcggtacacacgatgaagcataaagatgggaaaaatagactcatgaactttatctatgatcgctgttattaccatatgcaaggggaagaaatctttagcctcgcaagattttatgcaatccatcatgcaccaaagttgtttgacgttttttatgattgttgtatcctagatacgatacgattcaaaagccttcttttagattgttcacatatcataggtaaaaacgctcatgatgctaccaatatcaacatcgtgaacaagtatatcggcaacctgtttgttatgggagttcttagcaaaaaagaaatcttacaggactatccatccatttattctaaacaatacatgccttagtttattttttttgcggccgaaacattattcttaccctagaaaacgcttatagtcatcttaaatcataggtaaggaagatcatcatattttttgaaacgtaattttttaacgcatgatctatgatttcagggtccgtgcttttaggcaacggggtggtggccggactataaatctttagggataaaatgttctttataagctcatacccttcccctaaagctgtagtaccctcttcgaaaacatcagcccccagatctatacaaaagaacatgttttctatattatagtactgtattgagctaagcatggcttgattgatgttggcgcccaggacatagcagtagtacatggttgaaaggttgtggtctttgatgcaggcgatccgcatcatctcttctatgtccatatggatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttgagtagtaatgtactgagcgaaaactcattatttagcagatcggtttttactattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtttccttggcacctagctctcgacagaggtcccaggtgtgctccgtgttgacagataccagcccgtagttgatgtccgccccccactctgcaaacagttttataaggttgtagttgttttcccttacagccttcactaacgccgtatttaggtttaagccctctttaatacctgctgattttatgagccttaggttatgatcaaacgtgatcggagcatcatgccaccataggtcataacactttaaaagataatgttggttcgtgggcacgcattgtccagccaacacctttttggtcagagattgcagggaaggcaacatgtctcttcatcttttaaaaaaaaatcaaattaattagccgaataaatttttctttcgagggctttttaaaagagctctttaagagctctttaagagctttttaagagattaaaaaattattcttgctggcattctgccaagtatgcggcattcctatcatctatagtatattatgagaatattcccaaatgatggataagttttttgatttataatcttttaataaactgcttatttcttcggggtcctttaagtttagtggcaaggaagcatctgagctgtaaatatccaaagccaaactatggctcagaaaattataacctttttgttccgctatggcacgaccctcttcaaaggcattaccacccaaatctatacagaaaaatatattaccgatgttataatattgtactgaagtaagcatagcttggttgatgttgccccccagcgcgtaacagtaatatattgttaatggattgttatccttggtagaagccagacatatcatgtcatggacgtctatttggatgttttccttgtggtacatctcatgaagctcatatattttgttataatacaggagacattttaatcgccattcattaagatccgtatatttctcatctagaaaacaaatggcgtccttacaatcgtattgtactgctttggcgtaccaatacttcactagtaaaccatttaactcgtccgtttcttttatttctatgagcccccatagtcttttataaattaagccccttaattgtataacaaatttgttttctaaaataggattattcataaaaatttcatggcacaaaataatactgccgctggttttattgtgcattatcctggtaaaaatacggaaaatatcgttgtcctctagagtttctttggcgcctagctgtctacacaactctcggatgtgcttcgtattgatagaaagcaaaccatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacaatatttcaaaatgcagtgttcatctattgacaaacactggctggctatcgtctttttgacgagggtctgcagagagagcggcaacgacatgtttctttttcaccaaaaaaaatcaaatgttctcgtctttaaaggttaattcatgttcttaaaatgttcatttcatgatagtgattaataatatggtttaataacgctagaaggcttgtttataagacagtcataagcagtctataagacagtctataagcagtctataagacagtctatgacttagtctataactataatttctggatgggctgtaagatactcttcggctcgtttcagattttttgaagtatatgtctttagcatatcatatatttcctggggttcggttacatctaataccaaggtcacatcacggctgaaaagctgctttactaagaaaatgttgctcaagttatacatataagctttgtgcgcaatgagttgtgccctatcaaaatcggcagcccccaaatcaatacagaaaaacatgtttaaagtattattgttatagatagaaagattcatgccataatcgagactagcccccaacctatgacagtaataaatggccgcgtaatttttttcccgcaagcaagcaaatttcatcatcagattagggctgatgcaaatctctttttcacgacacaactcgtgtatgtcaaaaatgttattaaaataaaggctacaagctacccgccaatagaggtgatttttatgccttttatagaaatagtgaatagcctttgtaaaattatgtcgtaatgccagggcaaaccaaaactttgttaataggtggtgcgccgtatcccccgtcaacggaatgtttgaacaggtgtacgtaactgtgtctaaagtggttctagttacggtttccaagagtggattatgacaaaacatgtcataacccagcagaactcctgcacaggattttagcctggccacttcttttaaaatttccagaagacggggttcggatacaggcgttaagcctcccagttccgcacacagccgctttagatacacggcaggaacacgtataagcccatattcaggatttgcgccccaatccacaaataaacgtataagttcaagattatcgctcttcacggcctttactagcgccgcttcgagacaaagatcatcctcagaaaaacactgtaaatgttta

SEQ ID NO: 424 - protein, 254 aa, synthetic construct
  REGION 1..254: note = SYNTHETIC CONSTRUCT DERIVED FROM HUMAN THOUGHT
  VARIANT 29: note = MISC_FEATURE - Xaa29 IS Asp OR Asn
  REGION 29..195: note = MISC_FEATURE - AT LEAST ONE OF Xaa29, Xaa42, Xaa50, Xaa131, Xaa152, Xaa160, AND Xaa195 IS Asp
  VARIANT 42: note = MISC_FEATURE - Xaa42 IS Asp OR Asn
  VARIANT 50: note = MISC_FEATURE - Xaa50 IS Asp OR Asn
  VARIANT 131: note = MISC_FEATURE - Xaa131 IS Asp OR Asn
  VARIANT 152: note = MISC_FEATURE - Xaa152 IS Asp OR Asn
  VARIANT 160: note = MISC_FEATURE - Xaa160 IS Asp OR Asn
  VARIANT 195: note = MISC_FEATURE - Xaa195 IS Asp OR Asn
MVNSCTFLHIFLCCSFLYSFCCAVVAGSXTTYCFWFPLVRGXFSFELTVXYTVCPPCLTRQAATEIYEPGRSLWCRIGYDRCGEDDHDELGFMIPPGLSSEGHLTSVYAWLAFLSFSYTAQFHPEIFGIGXVSRVYVDIKHQLICAEHDGQXTTLPRHDXISAVFQTYYQHQVDGGNWFHLEWLRPFFSSWLVLXVSWFLRRSPANHVSVRVLQILRPTPPQRQALLSSKTSVALGIATRPLRRFAKSLSAVRR

SEQ ID NO: 425 - protein, 203 aa, synthetic construct
  REGION 1..203: note = SYNTHETIC CONSTRUCT DERIVED FROM HUMAN THOUGHT
ATVYDIGHDAVMYVAERKVSWAPRGGDEVKFEAVPGELKLIANRLRTSFPPHHTVDMSKFAFTAPGCGVSMRVERQHGCLPADTVPEGNCWWSLFDLLPLEVQNKEIRHANQFGYQTKHGVSGKYLQRRLQVNGLRAVTDLNGPIVVQYFSVKESWIRHLKLAGEPSYSGFEDLLRIRVEPDTSPLADKEEKIFRFGSHKWYG

SEQ ID NO: 426 - protein, 470 aa, synthetic construct
  REGION 1..470: note = SYNTHETIC CONSTRUCT DERIVED FROM HUMAN THOUGHT
  VARIANT 14: note = MISC_FEATURE - Xaa14 IS Asn OR Asp
  REGION 14..468: note = MISC_FEATURE - AT LEAST ONE OF Xaa14, Xaa96, AND Xaa468 IS Asp
  VARIANT 96: note = MISC_FEATURE - Xaa96 IS Asn OR Asp
  VARIANT 468: note = MISC_FEATURE - Xaa468 IS Asn OR Asp
SLCQVIEDCCCSQXKTNRVTPEEVAAKIDLYLRGATNLEECLARLEKARPPRVIDTSFDWDVVLPGVEAATQTIKLPQVNQCRALVPVVTQKSLDXNSVPLTAFSLANYYYRAQGDEVRHRERLTAVLSKLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQDEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRXTS

SEQ ID NO: 427 - protein, 111 aa, synthetic construct
  REGION 1..111: note = SYNTHETIC CONSTRUCT DERIVED FROM HUMAN THOUGHT
NGPNAWGDEDRLNKKKRRRMEALGIYVMGGKKYQKFWDKNSGDVFYEEVHDNTDEWECLRVGDPADFDPEKGTLCGHVTIENKAYHVYTSPSGKKFLVPVNPENGRVQWEA

SEQ ID NO: 428 - protein, 553 aa, synthetic construct
  REGION 1..553: note = SYNTHETIC CONSTRUCT DERIVED FROM HUMAN THOUGHT
  VARIANT 1: note = MISC_FEATURE - Xaa1 IS Asp OR Asn
  REGION 1..551: note = MISC_FEATURE - AT LEAST ONE OF Xaa1, Xaa158, AND Xa551 IS Asp
  VARIANT 158: note = MISC_FEATURE - Xaa158 IS Asp OR Asn
  VARIANT 551: note = MISC_FEATURE - Xaa551 IS Asp OR Asn
XRTFTLGPVNLKVASEVELKDAVEHNQHPVARPIDGGVVLLRSAVPSLIDVLISGADASPKLLAHHGPGNTGIDGTLWDFESEATKEEVALSAQIIQACDIRRGDAPEIGLPYKLYPVRGNPERVKGVLQNTRFGDIPYKTPSDTGSPVHAAACLTPXATPVTDGRSVLATTMPPGFELYVPTIPASVLDYLDSRPDCPKQLTEHGCEDAALKDLSKYDLSTQGFVLPGVLRLVRKYLFAHVGKCPPVHRPSTYPAKNSMAGINGNRFPTKDIQSVPEIDVLCAQAVRENWQTVTPCTLKKQYCGKKKTRTILGTNNFIALAHRAVLSGVTQGFMKKAFNSPIALGKNKFKELQTPVLGRCLEADLASCDRSTPAIVRWFAANLLYELACAEEHLPSYVLNCCHDLLVTQSGAVTKRGGLSSGDPITSVSNTIYSLVIYAQHMVLSYFKSGHPHGLLFLQDQLKFEDMLKVQPLIVYSDDLVLYAESPTMPNYHWWVEHLNLMLGFQTDPKKTAITDSPSFLGCRIINGRQLVPNRDRILAALAYHMKASXVS

SEQ ID NO: 429 - protein, 441 aa, synthetic construct
  REGION 1..441: note = SYNTHETIC CONSTRUCT BASED ON HUMAN THOUGHT
GKKSRVCGYCGAPAPYATACGLDVCIYHTHFHQHCPVTIWCGHPAGSGSCSECKSPVGKGTSPLDEVLEQVPYKPPRTVIMHVEQGLTPLDPGRYQTRRGLVSVRRGIRGNEVGLPDGDYASTALLPTCKEINMVAVASNVLRSRFIIGPPGAGKTYWLLQQVQDGDVIYTPTHQTMLDMIRALGTCRFNVPAGTTLQFPVPSRTGPWVRILAGGWCPGKNSFLDEAAYCNHLDVLRLLSKTTLTCLGDFKQLHPVGFDSHCYVFDIMPQTQLKTIWRFGQNICDAIQPDYRDKLMSMVDTTRVTYVEKPVRYGQVLTPYHRDREDDAITIDSSQGATFDVVTLHLPTKDSLNRQRALVAITRARHAIFVYDPHRQLQGLFDLPAKGTPVNLAVHCDGQLIVLDRNNKECTVAQALGNGDKFRATDKRVVDSLRAICADLE

SEQ ID NO: 430 - protein, 153 aa, synthetic construct
  REGION 1..153: note = SYNTHETIC CONSTRUCT DERIVED FROM HUMAN THOUGHT
GRYFTWYQLASYASYIRVPVDSTVYLDPCMGPALCNRRVVGSTHWGADLAVTPYDYGAKIILSSAYHGEMPPGYKILACAEFSLDDPVKYKHTWGFESDTAYLYEFTGNGEDWEDYNDAFRARQEGKIYKATATSLKFYFPPGPVIEPTLGLN

SEQ ID NO: 431 - protein, 2040 aa, synthetic construct
  REGION 1..2040: note = SYNTHETIC CONSTRUCT DERIVED FROM HUMAN THOUGHT
  VARIANT 1: note = MISC_FEATURE - Xaa1 IS Asp OR Asn
  REGION 1..2038: note = MISC_FEATURE - AT LEAST ONE OF Xaa1, Xaa264, Xaa346, Xaa718, Xaa1761, AND Xaa2038 IS Asp
  VARIANT 264: note = MISC_FEATURE - Xaa264 IS Asp OR Asn
  VARIANT 346: note = MISC_FEATURE - Xaa346 IS Asp OR Asn
  VARIANT 718: note = MISC_FEATURE - Xaa718 IS Asp OR Asn
  VARIANT 1761: note = MISC_FEATURE - Xaa1761 IS Asp OR Asn
  VARIANT 2038: note = MISC_FEATURE - Xaa2038 IS Asp OR Asn
XTSPLADKEEKIFRFGSHKWYGAGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQXKTNRVTPEEVAAKIDLYLRGATNLEECLARLEKARPPRVIDTSFDWDVVLPGVEAATQTIKLPQVNQCRALVPVVTQKSLDXNSVPLTAFSLANYYYRAQGDEVRHRERLTAVLSKLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQDEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRXTSVYQAICTLDGRLKFLPKMILETPPPYPCEFVMMPHTPAPSVGAESDLTIGSVATEDVPRILEKIENVGEMANQGPLAFSEDKPVDDQLVNDPRISSRRPDESTSAPSAGTGGAGSFTDLPPSDGADADGGGPFRTVKRKAERLFDQLSRQVFDLVSHLPVFFSRLFYPGGGYSPGDWGFAAFTLLCLFLCYSYPAFGIAPLLGVFSGSSRRVRMGVFGCWLAFAVGLFKPVSDPVGAACEFDSPECRNILHSFELLKPWDPVRSLVVGPVGLGLAILGRLLGGARCIWHFLLRLGIVADCILAGAYVLSQGRCKKCWGSCIRTAPNEVAFNVFPFTRATRSSLIDLCDRFCAPKGMDPIFLATGWRGCWAGRSPIEQPSEKPIAFAQLDEKKITARTVVAQPYDPNQAVKCLRVLQAGGAMVAKAVPKVVKVSAVPFRAPFFPTGVKVDPDCRVVVDPDTFTAALRSGYSTTNLVLGVGDFAQLNGLKIRQISKPSGGGPHLMAALHVACSMALHMLAGIYVTAVGSCGTGTNDPWCANPFAVPGYGPGSLCTSRLCISQHGLTLPLTALVAGFGIQEIALVVLIFVSIGGMAHRLSCKADMLCVLLAIASYVWVPLTWLLCVFPCWLRCFSLHPLTILWLVFFLISVNMPSGILAMVLLVSLWLLGRYTNVAGLVTPYDIHHYTSGPRGVAALAIAPDGTYLAAVRRAALTGRTMLFTPSQLGSLLEGAFRTRKPSLNTVNVIGSSMGSGGVFTIDGKVKCVTAAHVLTGNSARVSGVGFNQMLDFDVKGDFAIADCPNWQGAAPKTQFCTDGWTGRAYWLTSSGVEPGVIGKGFAFCFTACGDSGSPVITEAGELVGVHTGSNKQGGGIVTRPSGQFCNVAPIKLSELSEFFAGPKVPLGDVKVGSHIIKDISEVPSDLCALLAAKPELEGGLSTVQLLCVFFLLWRMMGHAWTPLVAVSFFILNEVLPAVLVRSVFSFGMFVLSWLTPWSAQVLMIRLLTAALNRNRWSLAFFSLGAVTGFVADLAATQGHPLQAVMXLSTYAFLPRMMVVTSPVPVITCGVVHLLAIILYLFKYRGPHHILVGDGVFSAAFFLRYFAEGKLREGVSQSCGMNHESLTGALAMRLNDEDLDFLMKWTNFKCFVSASNMRNAAGQFIEAAYAKALRVELAQLVQVDKVRGTLAKLEAFADTVAPQLSPGDIVVALGHTPVGSIFDLKVGSTKHTLQAIETRVLAGSKMTVARVVDPTPTPPPAPVPIPLPPKVLENGPNAWGDEDRLNKKKRRRMEALGIYVMGGKKYQKFWDKNSGDVFYEEVHXNT

SEQ ID NO: 432 - protein, 1295 aa, synthetic construct
  REGION 1..1295: note = SYNTHETIC CONSTRUCT DERIVED FROM HUMAN THOUGHT
  VARIANT 1: note = MISC_FEATURE - Xaa1 IS Asp OR Asn
  REGION 1..1293: note = MISC_FEATURE - AT LEAST ONE OF Xaa1, Xaa556, Xaa908, AND Xaa1293 IS Asp
  VARIANT 556: note = MISC_FEATURE - Xaa556 IS Asp OR Asn
  VARIANT 908: note = MISC_FEATURE - Xaa908 IS Asp OR Asn
  VARIANT 1293: note = MISC_FEATURE - Xaa1293 IS Asp OR Asn
XRTFTLGPVNLKVASEVELKDAVEHNQHPVARPIDGGVVLLRSAVPSLIDVLISGADASPKLLAHHGPGNTGIDGTLWDFESEATKEEVALSAQIIQACDIRRGDAPEIGLPYKLYPVRGNPERVKGVLQNTRFGDIPYKTPSDTGSPVHAAACLTPTDGRSVLATTMPPGFELYVPTIPASVLDYLDSRPDCPKQLTEHGCEDAALKDLSKYDLSTQGFVLPGVLRLVRKYLFAHVGKCPPVHRPSTYPAKNSMAGINGNRFPTKDIQSVPEIDVLCAQAVRENWQTVTPCTLKKQYCGKKKTRTILGTNNFIALAHRAVLSGVTQGFMKKAFNSPIALGKNKFKELQTPVLGRCLEADLASCDRSTPAIVRWFAANLLYELACAEEHLPSYVLNCCHDLLVTQSGAVTKRGGLSSGDPITSVSNTIYSLVIYAQHMVLSYFKSGHPHGLLFLQDQLKFEDMLKVQPLIVYSDDLVLYAESPTMPNYHWWVEHLNLMLGFQTDPKKTAITDSPSFLGCRIINGRQLVPNRDRILAALAYHMKASXVSEYYASAAAILMDSCACLEYDPEWFEELVVGIAQCARKDGYSFPGTPFFMSMWEKLRSNYEGKKSRVCGYCGAPAPYATACGLDVCIYHTHFHQHCPVTIWCGHPAGSGSCSECKSPVGKGTSPLDEVLEQVPYKPPRTVIMHVEQGLTPLDPGRYQTRRGLVSVRRGIRGNEVELPDGDYASTALLPTCKEINMVAVASNVLRSRFIIGPPGAGKTYWLLQQVQDGDVIYTPTHQTMLDMIRALGTCRFNVPAGTTLQFPVPSRTGPWVRILAGGWCPGKNSFLDEAAYCNHLDVLRLLSKTTLTCLGDFKQLHPVGFDSHCYVFDIMPQTQLKTIWRFGQNICDAIQPDYRDKLMSMVXTTRVTYVEKPVRYGQVLTPYHRDREDDAITIDSSQGATFDVVTLHLPTKDSLNRQRALVAITRARHAIFVYDPHRQLQGLFDLPAKGTPVNLAVHRDGQLIVLDRNNKECTVAQALGNGDKFRATDKRVVDSLRAICADLEGSSSPLPKVAHNLGFYFSPDLTQFAKLPVELAPHWPVVTTQNNEKWPDRLVASLRPIHKYSRACIGAGYMVGPSVFLGTPGVVSYYLTKFVKGEAQVLPETVFSTGRIEVDCREYLDDREREVAASLPHAFIGDVKGTTVGGCHHVTSRYLPRVLPKESVAVVGVSSPGKAAKALCTLTDVYLPDLEAYLHPETQSKCWKMMLDFKEVRLMVWKDKTAYFQLEGRYFTWYQLASYASYIRVPVXST

SEQ ID NO: 433 - protein, 116 aa, synthetic construct
  REGION 1..116: note = SYNTHETIC CONSTRUCT DERIVED FROM HUMAN THOUGHT
  VARIANT 38: note = MISC_FEATURE - Xaa38 IS Asp OR Asn
  REGION 38..44: note = MISC_FEATURE - AT LEAST ONE OF Xaa38 AND Xaa44 IS Asp
  VARIANT 44: note = MISC_FEATURE - Xaa44 IS Asp OR Asn
SLDVVAHFQHLAAIEAETCKYLASRLPMLHNLRMTGSXVTIVYXSTLNQVFAIFPTPGSRPKLHDFQQWLIAVHSSIFSSVAASCTLFVVLWLRVPILRTVFGFRWLGAIFLSNSQ

SEQ ID NO: 434 - protein, 254 aa, synthetic construct; identical to SEQ ID NO: 424
  REGION 1..254: note = SYNTHETIC CONSTRUCT DERIVED FROM HUMAN THOUGHT
  VARIANT 29: note = MISC_FEATURE - Xaa29 IS Asp OR Asn
  REGION 29..195: note = MISC_FEATURE - AT LEAST ONE OF Xaa29, Xaa42, Xaa50, Xaa131, Xaa152, Xaa160, AND Xaa195 IS Asp
  VARIANT 42: note = MISC_FEATURE - Xaa42 IS Asp OR Asn
  VARIANT 50: note = MISC_FEATURE - Xaa50 IS Asp OR Asn
  VARIANT 131: note = MISC_FEATURE - Xaa131 IS Asp OR Asn
  VARIANT 152: note = MISC_FEATURE - Xaa152 IS Asp OR Asn
  VARIANT 160: note = MISC_FEATURE - Xaa160 IS Asp OR Asn
  VARIANT 195: note = MISC_FEATURE - Xaa195 IS Asp OR Asn

SEQ ID NO: 435 - protein, 178 aa, synthetic construct
  REGION 1..178: note = SYNTHETIC CONSTRUCT DERIVED FROM HUMAN THOUGHT
  VARIANT 37: note = MISC_FEATURE - Xaa37 IS Asp OR Asn
  REGION 37..130: note = MISC_FEATURE - AT LEAST ONE OF Xaa37, Xaa84, Xaa120, AND Xaa130 IS Asp
  VARIANT 84: note = MISC_FEATURE - Xaa84 IS Asp OR Asn
  VARIANT 120: note = MISC_FEATURE - Xaa120 IS Asp OR Asn
  VARIANT 130: note = MISC_FEATURE - Xaa130 IS Asp OR Asn
MASSLLFLVVGFKCLLVSQAFACKPCFSSSLADIKTXTTAAASFAVLQDISCLRHRDSASEAIRKIPQCRTAIGTPVYVTITAXVTDENYLHSSDLLMLSSCLFYASEMSEKGFKVVFGXVSGIVAVCVXFTSYVQHVKEFTQRSLVVDHVRLLHFMTPETMRWATVLACLFAILLAI

SEQ ID NO: 436 - protein, 70 aa, synthetic construct
  REGION 1..70: note = SYNTHETIC CONSTRUCT DERIVED FROM HUMAN THOUGHT
  VARIANT 30: note = MISC_FEATURE - Xaa30 IS Asp OR Asn
  REGION 30..51: note = MISC_FEATURE - AT LEAST ONE OF Xaa30, Xaa33, Xaa44, AND Xaa51 IS Asp
  VARIANT 33: note = MISC_FEATURE - Xaa33 IS Asp OR Asn
  VARIANT 44: note = MISC_FEATURE - Xaa44 IS Asp OR Asn
  VARIANT 51: note = MISC_FEATURE - Xaa51 IS Asp OR Asn
MLEKCLTAGCCSRLLSLWCIVPFCFAVLAXASXDSSSHLQLIYXLTLCELXGTDWLANKFDWAVESFVIF

SEQ ID NO: 437 - protein, 174 aa, synthetic construct
  REGION 1..174: note = SYNTHETIC CONSTRUCT DERIVED FROM HUMAN THOUGHT
MGSSLDDFCHDSTAPQKVLLAFSITYTPVMIYALKVSRGRLLGLLHLLIFLNCAFTFGYMTFAHFQSTNKVALTMGAVVALLWGVYSAIETWKFITSRCRLCLLGRKYILAPAHHVESAAGFHPIAANDNHAFVVRRPGSTTVDGTLVPGLKSLVLGGRKAVKQGVVNLVKYAK

SEQ ID NO: 438 - protein, 123 aa, synthetic construct
  REGION 1..123: note = SYNTHETIC CONSTRUCT DERIVED FROM HUMAN THOUGHT
MPNNNGKQQKRKKGDGQPVNQLCQMLGKIIAQQDQSRGKGPGKKNKKKNPEKPHFPLATEDDVRHHFTPSERQLCLSSIQTAFNQGAGTCTLSDSGRISYTVEFSLPTHHTVRLIRVTASPSA

SEQ ID NO: 439 - protein, 296 aa, synthetic construct
  REGION 1..296: note = SYNTHETIC CONSTRUCT DERIVED FROM HUMAN THOUGHT
VRRESPKHRWCFTINDWTPTEWESIVECGGSIARYLIIGKEVGKSGTPHLQGYVNFKNKRRLSSVKRLPGFGRAHLEPARGSHKEASEYCKKEGDYLEIGEDSSSGTRSDLQAAARILTETSGNLTEVAEKMPAVFIRYGRGLRDFCGVMGLGKPRDFKTEVYVFIGPPGCGKTREACADAAARELQLYFKPRGPWWDGYNGEGAVILDDFYGWVPFDELLRIGDRYPLRVPVKGGFVNFVAKVLYITSNVVPEEWYSSENIRGKLEALFRRFTKVVFWGEGGVKKDMETVYPINY

SEQ ID NO: 440 - protein, 296 aa, synthetic construct
  REGION 1..296: note = SYNTHETIC CONSTRUCT DERIVED FROM HUMAN THOUGHT
VRRESPKHRWCFTINNWTPTEWESIVECGGSIARYLIIGKEVGKSGTPHLQGYVNFKNKRRLSSVKRLPGFGRAHLEPARGSHKEASEYCKKEGDYLEIGEDSSSGTRSDLQAAARILTETSGDLTEVAEKMPAVFIRYGRGLRDFCGVMGLGKPRDFKTEVYVFIGPPGCGKTREACADAAARELQLYFKPRGPWWDGYNGEGAVILDDFYGWVPFDELLRIGDRYPLRVPVKGGFVNFVAKVLYITSNVVPEEWYSSENIRGKLEALFRRFTKVVFWGEGGVKKDMETVYPINY

SEQ ID NO: 441 - protein, 296 aa, synthetic construct; identical to SEQ ID NO: 440
  REGION 1..296: note = SYNTHETIC CONSTRUCT DERIVED FROM HUMAN THOUGHT

SEQ ID NO: 442 - protein, 245 aa, synthetic construct
  REGION 1..245: note = SYNTHETIC CONSTRUCT DERIVED FROM HUMAN THOUGHT
MFKWLMSSLCGIRDSTSPEVYEPIIGGQNPATMLRLQSALAAVNALLPATLTIEDVISSADNTRRLVKAQTLARTYQACQHNIECLSRHRASSDNPNLNAVVTTHMINAKRLSDTCLAALMHLYLSVGAVDATTDTMVDHAIRMTAENSVVMADVAVLEKTLGLDPQATVRAQDLLALNSGVLNSVNAVAEMTDPTDDVEFTQSVHSPLLPRQLSTTEVVGVPSPVKSNLKSKHKPKRKASLVAV

SEQ ID NO: 443 - protein, 497 aa, synthetic construct
  REGION 1..497: note = SYNTHETIC CONSTRUCT DERIVED FROM HUMAN THOUGHT
MGLFGLLKYAYSNRLVKHDAITTPPGIMTPIAIDLWNVMYTLMEKFDQERNFPLDGAAVTARCFFSLLRLLLKRSYYPIFVSDRGIYGDGRVKQGAKAIVSQTMSSYGGSGRLSSACFTGDEHDTEFQEDPEENDVSVPPQDTCPPTEISAGYVEPERKCEHSSTRWSALDGAPRLSYRLCVNLIRHLGYPYVNACNLEADDVCANLYHTNTVAQIYTTDTDLILMGCDIILDIMPLFPPTLRCCDVLMDLGVTYDEFLTEFVRCHTDLHEPQTLASVQSVISSLHSPPDEDEGADMPQTPSGHSWRCPNERRVISWRRQDDHDYDSSTEDSDQSDSSEEEEECPAGKGFGYRENPAVETCKRRTRPRRSAEASGRILHLKYTSRYPPIMESAPRALVRMAPPKTRHEVLERKFVKHVVSMLTPERRGSLSIMRRLPITQEPSDFSLVHDTLKNLVSEHEIARELANMFWNHIPTPTDYNTVLVNYWDDCGHRRQWS

SEQ ID NO: 444 - protein, 275 aa, synthetic construct
  REGION 1..275: note = SYNTHETIC CONSTRUCT DERIVED FROM HUMAN THOUGHT
MDSYNYRDFAVGGGLLQRIRLVVSGSLHCGESDATLNDPKHLPARCVFQFSGPDDNSVTFPIEYVLRLMKNWARSQCDPYIRIQNTGVSVLFQGFFFAPPNAPMASITSEHNNVILKSTHTTGLALSGIERVKRGGGLDLRPLQAMMQISCFTRMPVVQLSFRFMGPEDASRTQRLLERATSFGAMELHQKRTVDSCDRSNGIVSPREHRECRERQKRRPTPKRCASEVFASLASISSAFASERVKRRPVRIAAAILAFVFVAVILAIATKGRLF

SEQ ID NO: 445 - protein, 162 aa, synthetic construct
  REGION 1..162: note = SYNTHETIC CONSTRUCT DERIVED FROM HUMAN THOUGHT
MYGRSNAPGCQPPVRAHPPSSPMTSRLQLAAMEQDASDAALSGMQQARSRQASPKRLRDLIPSSDVESMSLNALDDAYGHHDFSVWFEDLMPPEMELLMPTTDAKLNYLSFTRRLASSVFHSPDNRMPGGACDHTSALDARKERFATIINKFLDLHQILHDA

SEQ ID NO: 446 - protein, 326 aa, synthetic construct
  REGION 1..326: note = SYNTHETIC CONSTRUCT DERIVED FROM HUMAN THOUGHT
MFDGRSDIYDSTSFAAELDDLYSCRSTGRENGRRSRVSTRGVHRDRCGSAAKRRSTKRRCELVARERDRYSLYLDYMASHPSDEISAVRELVVPLIKTTSITLPFDLDQTVADNCLSLSGMGYYLGIGGCCPTCTVSGEPRLHRADRAALILAYVQQLNNIYEYRGFLASVLAAAAQGDQAGVAASEGVQAERLLENVLAQPELFFAYHVLRDGGIQNVRVLFYRDLSVSGYMMYAVFPTKSVHLHYRLIDRLLAACPGYKIIAHVWQTAFVLVVRRDEGQQTDMDIPTVSAGDIYCKMCDLSFDGELLLEYKKLYAVFDDFLPPV

SEQ ID NO: 447 - protein, 272 aa, synthetic construct
  REGION 1..272: note = SYNTHETIC CONSTRUCT DERIVED FROM HUMAN THOUGHT
MKRRQRLTARSRLRAGIRCHNRFYNAMVQDLASAKRNGVYGERLAPLFSELVPAETLKTALGVSLAFEVNLGQRRPDCVCTVQFGKGSDAKGVCILIELKTCRFSKNMNTASKNLQRKGGMRQLHDSCRLLARTLPPGSGEIVLAPVLVFVAQRGMRVLRVTRLSPQVVYSNAAVLSCTISRLAEYAPPVSAKSTRRRCVAKGTKAKAFSTKAAAEPVPSITPAQPSAAAAVVSLFPAAVPADTTNAAAVHQPVAVSHVNPLAWAASLFSPK

SEQ ID NO: 448 - protein, 370 aa, synthetic construct
  REGION 1..370: note = SYNTHETIC CONSTRUCT DERIVED FROM HUMAN THOUGHT
MMAAASDSCLSLWEGSASSPNRQLTPEAVNCLTEALTEDVAVLRLIRSDPRVKIFMAVSVLTPRLARFAPPPPKLTHTAKCAVIMIYLTRPKALALQPKQFHMLVTFNKASVYSLVVRVKTKPFPVGTQRFRAVFQDPEFIGLPSDIPDPAAENIPTEINDRLDVSNFATPAQPPKDKYDCCVLAPGVWWSNANKAIYFLQMDVALLALCPAGWKARGLGIILGRLLNHQEGCATCRFTEHSDPLDATADSVATPESCLCWAPCLWRKAHQRELTVEGDRYLFRVLFMDAVERVRLTGLRRSPKITANLADLVVGIGPHGQQIPVNNAGWKLVALDADISRLIVCGCYALRYICPPTNSKHQPSSPDEYA

SEQ ID NO: 449 - protein, 450 aa, synthetic construct
  REGION 1..450: note = SYNTHETIC CONSTRUCT DERIVED FROM HUMAN THOUGHT
MARRGAAVAEEPLLPSSGIVGIGPIEGINWRTWLVQVFCFALTTSVLFITLVTASLPQTGYPCFYGSLVDYTQKDHSVVDGVWMRQIAGGVAPTLFLETTSLVAFLYYTTLVLVAISFYLIISAVLVRRYARGKECTAVAGCTRPTTTLIASHVTLVLGTLATWLLQVVILLLSHKQAVLGAAVYVVHFVSLVFFCMSFSGLGTASAQYSSNLRILKTNLPALHKMAGPGRAVMTNLGMGMLGISLPILSLMLGIILANSFHITLWQTVTVAVGVFVALGLMFLIIVELIVSHYVHVLVGPALAVLVASSTLAVATHSYFVHFHAMVSVQAPNLATASKAIVGIMAVISIIMLVVRLVRAIMFHKKRNTEFYGRVKTVSSKARRYANKVRGPRRNPQPLNVAESRGMLLAEDSETDAEEPIYDVVSEEFETEYYDDPQRVPERSHRREYR

SEQ ID NO: 450 - protein, 130 aa, synthetic construct
  REGION 1..130: note = SYNTHETIC CONSTRUCT DERIVED FROM HUMAN THOUGHT
MSSNSDNTECFGGVNYAEGMRKRKRNPVRDSTFQEYLDARNARYPRSGSTSDSDEDYTTRSKYESDVSEFKKMMDLETLPPPKAEPQAQKAEPDAAKEEPVSTTSYILNEWVAPMIGHFLAMCMYELLFK

SEQ ID NO: 451 - protein, 617 aa, synthetic construct
  REGION 1..617: note = SYNTHETIC CONSTRUCT DERIVED FROM HUMAN THOUGHT
MLDFHGISLTCAAVASSKVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASIAETALTSGSSPSAPPSDSPGPPILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATEKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 452 - protein, 763 aa, Canis lupus
MAALSGGGGAEQGQALFNGDMEPEAGAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDISRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASIAETALTSGSSPSAPPSDSPGPPILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 453 - protein, 749 aa, Canis lupus
QALFNGDMEPEAGAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDISRSNPKSPQKPIVRVFLPNKQRTVVPARCGVTVRDSLKKALMMRGLIPECCAVYRIQDGEKKPIGWDTDISWLTGEELHVEVLENVPLTTRNFVRKTFFTLAFCDFCRKLLFQGFRCQTCGYKFHQRCSTEVPLMCVNYDQLDLLFVSKFFEHHPIPQEEASIAETALTSGSSPSAPPSDSPGPPILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 454 - protein, 766 aa, Homo sapiens [sequence omitted]

SEQ ID NO: 455 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 456 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 457 - protein, 1259 aa, Canis lupus
MELAAWCRWGLLLALLPSGAAGTQVCTGTDMKLRLPASPETHLDMLRHLYQGCQVVQGNLELTYLPANASLSFLQDIQEVQGYVLIAHSQVRQIPLQRLRIVRGTQLFEDNYALAVLDNGDPLEGGIPAPGAAQGGLRELQLRSLTEILKGGVLIQRSPQLCHQDTILWKDVFHKNNQLALTLIDTNRFSACPPCSPACKDAHCWGASSGDCQSLTRTVCAGGCARCKGPQPTDCCHEQCAAGCTGPKHSDCLACLHFNHSGICELHCPALVTYNTDTFESMPNPEGRYTFGASCVTSCPYNYLSTDVGSCTLVCPLNNQEVTAEDGTQRCEKCSKPCARVCYGLGMEHLREVRAVTSANIQEFAGCKKIFGSLAFLPESFDGDPASNTAPLQPEQLRVFEALEEITGYLYISAWPDSLPNLSVFQNLRVIRGRVLHDGAYSLTLQGLGISWLGLRSLRELGSGLALIHRNARLCFVHTVPWDQLFRNPHQALLHSANRPEEECVGEGLACYPCAHGHCWGPGPTQCVNCSQFLRGQECVEECRVLQGLPREYVKDRYCLPCHSECQPQNGSVTCFGSEADQCVACAHYKDPPFCVARCPSGVKPDLSFMPIWKFADEEGTCQPCPINCTHSCADLDEKGCPAEQRASPVTSIIAAVVGILLAVVVGLVLGILIKRRRQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVREHRGRLGSQDLLNWCVQIAKGMSYLEDVRLVHRDLAARNVLVKSPNHVKITDFGLARLLDIDETEYHADGGKVPIKWMALESIPPRRFTHQSDVWSYGVTVWELMTFGAKPYDGIPAREIPDLLEKGERLPQPPICTIDVYMIMVKCWMIDSECRPRFRELVAEFSRMARDPQRFVVIQNEDLGPASPLDSTFYRSLLEDDDMGDLVDAEEYLVPQQGFFCPEPTPGAGGTAHRRHRSSSTRNGGGELTLGLEPSEEEPPKSPLAPSEGAGSDVFDGDLGMGAAKGLQSLPSQDPSPLQRYSEDPTVPLPPETDGKVAPLTCSPQPEYVNQPEVWPQPPLALEGPLPPSRPAGATLERPKTLSPKTLSPGKNGVVKDVFAFGSAVENPEYLAPRGRAAPQPHPPPAFSPAFDNLYYWDQDPSERGSPPSTFEGTPTAENPEYLGLDVPV

SEQ ID NO: 458 - protein, 13 aa, synthetic construct
  REGION 1..13: note = SYNTHETIC CONSTRUCT DERIVED FROM HUMAN THOUGHT
GCCGCCACCATGG

SEQ ID NO: 459 - protein, 5 aa, synthetic construct
  REGION 1..5: note = SYNTHETIC CONSTRUCT DERIVED FROM HUMAN THOUGHT
GGGGS

SEQ ID NO: 460 - protein, 4 aa, synthetic construct
  REGION 1..4: note = SYNTHETIC CONSTRUCT DERIVED FROM HUMAN THOUGHT
GGGS

SEQ ID NO: 461 - protein, 4 aa, synthetic construct
  REGION 1..4: note = SYNTHETIC CONSTRUCT DERIVED FROM HUMAN THOUGHT
SGGG

SEQ ID NO: 462 - protein, 11 aa, synthetic construct
  REGION 1..11: note = SYNTHETIC CONSTRUCT DERIVED FROM HUMAN THOUGHT
GSGGGTGGGSG

SEQ ID NO: 463 - protein, 6 aa, synthetic construct
  REGION 1..6: note = SYNTHETIC CONSTRUCT DERIVED FROM HUMAN THOUGHT
GSGSGS

SEQ ID NO: 464 - protein, 10 aa, synthetic construct
  REGION 1..10: note = SYNTHETIC CONSTRUCT DERIVED FROM HUMAN THOUGHT
GSGSGSGSGS

SEQ ID NO: 465 - protein, 8 aa, synthetic construct
  REGION 1..8: note = SYNTHETIC CONSTRUCT DERIVED FROM HUMAN THOUGHT
GGGSGGGS

SEQ ID NO: 466 - protein, 10 aa, synthetic construct
  REGION 1..10: note = SYNTHETIC CONSTRUCT DERIVED FROM HUMAN THOUGHT
GGGGSGGGGS

SEQ ID NO: 467 - protein, 13 aa, synthetic construct
  REGION 1..13: note = SYNTHETIC CONSTRUCT DERIVED FROM HUMAN THOUGHT
GGGSGGGGSGGGS

SEQ ID NO: 468 - protein, 4 aa, synthetic construct
  REGION 1..4: note = SYNTHETIC CONSTRUCT DERIVED FROM HUMAN THOUGHT
GGSG

SEQ ID NO: 469 - protein, 8 aa, synthetic construct
  REGION 1..8: note = SYNTHETIC CONSTRUCT DERIVED FROM HUMAN THOUGHT
GGSGGGSG

SEQ ID NO: 470 - protein, 12 aa, synthetic construct
  REGION 1..12: note = SYNTHETIC CONSTRUCT DERIVED FROM HUMAN THOUGHT
GGSGGGSGGGSG

SEQ ID NO: 471 - protein, 6 aa, synthetic construct
  REGION 1..6: note = SYNTHETIC CONSTRUCT DERIVED FROM HUMAN THOUGHT
GGSGGS

SEQ ID NO: 472 - protein, 5 aa, synthetic construct
  REGION 1..5: note = SYNTHETIC CONSTRUCT DERIVED FROM HUMAN THOUGHT
SGGGG

SEQ ID NO: 473 - protein, 12 aa, synthetic construct
  REGION 1..12: note = SYNTHETIC CONSTRUCT DERIVED FROM HUMAN THOUGHT
SGGGGSGGGGIG

SEQ ID NO: 474 - protein, 10 aa, synthetic construct
  REGION 1..10: note = SYNTHETIC CONSTRUCT DERIVED FROM HUMAN THOUGHT
SGGGGSGGGG

SEQ ID NO: 475 - protein, 4 aa, synthetic construct
  REGION 1..4: note = SYNTHETIC CONSTRUCT DERIVED FROM HUMAN THOUGHT
AAAA

SEQ ID NO: 476 - protein, 5 aa, synthetic construct
  REGION 1..5: note = SYNTHETIC CONSTRUCT DERIVED FROM HUMAN THOUGHT
EAAAK

SEQ ID NO: 477 - protein, 12 aa, synthetic construct
  REGION 1..12: note = SYNTHETIC CONSTRUCT DERIVED FROM HUMAN THOUGHT
AEAAAKEAAAKA

SEQ ID NO: 478 - protein, 13 aa, synthetic construct
  REGION 1..13: note = SYNTHETIC CONSTRUCT DERIVED FROM HUMAN THOUGHT
EAEAAAKEAAAKA

SEQ ID NO: 479 - protein, 7 aa, synthetic construct
  REGION 1..7: note = SYNTHETIC CONSTRUCT DERIVED FROM HUMAN THOUGHT
EPKSCDK

SEQ ID NO: 480 - protein, 7 aa, synthetic construct
  REGION 1..7: note = SYNTHETIC CONSTRUCT DERIVED FROM HUMAN THOUGHT
IEGRDMD

SEQ ID NO: 481 - protein, 5 aa, synthetic construct
  REGION 1..5: note = SYNTHETIC CONSTRUCT DERIVED FROM HUMAN THOUGHT
PAPAP

SEQ ID NO: 482 - protein, 23 aa, synthetic construct
  REGION 1..23: note = SYNTHETIC CONSTRUCT DERIVED FROM HUMAN THOUGHT
ITPNGSIPNDKPFQNVNKITYGA

SEQ ID NO: 483 - protein, 4 aa, synthetic construct
  REGION 1..4: note = SYNTHETIC CONSTRUCT DERIVED FROM HUMAN THOUGHT
GFLG

SEQ ID NO: 484 - protein, 4 aa, synthetic construct
  REGION 1..4: note = SYNTHETIC CONSTRUCT DERIVED FROM HUMAN THOUGHT
ALAL

SEQ ID NO: 485 - protein, 9 aa, synthetic construct
  REGION 1..9: note = SYNTHETIC CONSTRUCT DERIVED FROM HUMAN THOUGHT
VDGGSGGGS

SEQ ID NO: 486 - protein, 16 aa, synthetic construct
  REGION 1..16: note = SYNTHETIC CONSTRUCT DERIVED FROM HUMAN THOUGHT
GGGGSLVPRGSGGGGS

SEQ ID NO: 487 - protein, 12 aa, synthetic construct
  REGION 1..12: note = SYNTHETIC CONSTRUCT DERIVED FROM HUMAN THOUGHT
SGGGGSGGGGIG

SEQ ID NO: 488 - protein, 4 aa, synthetic construct
  REGION 1..4: note = SYNTHETIC CONSTRUCT DERIVED FROM HUMAN THOUGHT
RVKR

SEQ ID NO: 489 - protein, 4 aa, synthetic construct
  REGION 1..4: note = SYNTHETIC CONSTRUCT DERIVED FROM HUMAN THOUGHT
VRVV

SEQ ID NO: 490 - protein, 25 aa, synthetic construct
  REGION 1..25: note = SYNTHETIC CONSTRUCT DERIVED FROM HUMAN THOUGHT
SSVSPTTSVHPTPTSVPPTPTKSSP

SEQ ID NO: 491 - protein, 25 aa, synthetic construct
  REGION 1..25: note = SYNTHETIC CONSTRUCT DERIVED FROM HUMAN THOUGHT
PTSTPADSSTITPTATPTATPTIKG

SEQ ID NO: 492 - protein, 25 aa, synthetic construct
  REGION 1..25: note = SYNTHETIC CONSTRUCT DERIVED FROM HUMAN THOUGHT
TVTPTATATPSAIVTTITPTATTKP

SEQ ID NO: 493 - protein, 25 aa, synthetic construct
  REGION 1..25: note = SYNTHETIC CONSTRUCT DERIVED FROM HUMAN THOUGHT
TNGSITVAATAPTVTPTVNATPSAA

SEQ ID NO: 494 - protein, 17 aa, Homo sapiens
VSQTSKLTRAETVFPDV

SEQ ID NO: 495 - protein, 6 aa, Homo sapiens
PLGLWA

SEQ ID NO: 496 - protein, 10 aa, Homo sapiens
EDVVCCSMSY

SEQ ID NO: 497 - protein, 8 aa, Homo sapiens
GGIEGRGS

SEQ ID NO: 498 - protein, 10 aa, Homo sapiens
TRHRQPRGWE

SEQ ID NO: 499 - protein, 10 aa, Homo sapiens
AGNRVRRSVG

SEQ ID NO: 500 - protein, 9 aa, Homo sapiens
RRRRRRRRR

SEQ ID NO: 501 - protein, 4 aa, Homo sapiens
GFLG

SEQ ID NO: 502 - protein, 4 aa, Homo sapiens
IEGR

SEQ ID NO: 503 - protein, 6 aa, Homo sapiens
LVPRGS

SEQ ID NO: 504 - protein, 5 aa, Homo sapiens
VIAGR

SEQ ID NO: 505 - protein, 19 aa, Porcine teschovirus-1
ATNFSLLKQAGDVEENPGP

SEQ ID NO: 506 - protein, 20 aa, Equine rhinitis A virus
QCTNYALLKLAGDVESNPGP

SEQ ID NO: 507 - protein, 22 aa, Foot-and-mouth disease virus
VKQTLNFDLLKLAGDVESNPGP

SEQ ID NO: 508 - protein, 18 aa, Thosea asigna virus
EGRGSLLTCGDVEENPGP

SEQ ID NO: 509 - protein, 5 aa, Homo sapiens
KKKRK

SEQ ID NO: 510 - protein, 26 aa, Homo sapiens
MLRTSSLFTRRVQPSLFRNILRLQST

SEQ ID NO: 511 - protein, 4 aa, Homo sapiens
KDEL

SEQ ID NO: 512-513 - skipped (2 entries)

SEQ ID NO: 514 - protein, 5 aa, Homo sapiens
KFERQ

SEQ ID NO: 515 - skipped

SEQ ID NO: 516 - protein, 4 aa, Homo sapiens
YQRL

SEQ ID NO: 517 - protein, 47 aa, synthetic construct
  REGION 1..47: note = SYNTHETIC CONSTRUCT DERIVED FROM HUMAN THOUGHT
LEIRAAFLRQRNTALRTEVAELEQEVQRLENEVSQYETRYGPLGGGK

SEQ ID NO: 518 - protein, 47 aa, synthetic construct
  REGION 1..47: note = SYNTHETIC CONSTRUCT DERIVED FROM HUMAN THOUGHT
LEIEAAFLERENTALETRVAELRQRVQRLRNRVSQYRTRYGPLGGGK

SEQ ID NO: 519 - protein, 33 aa, synthetic construct
  REGION 1..33: note = SYNTHETIC CONSTRUCT DERIVED FROM HUMAN THOUGHT
RMKQIEDKIEEILSKIYHIENEIARIKKLIGER

SEQ ID NO: 520 - protein, 32 aa, synthetic construct
  REGION 1..32: note = SYNTHETIC CONSTRUCT DERIVED FROM HUMAN THOUGHT
LSSIEKKQEEQTSWLIWISNELTLIRNELAQS

SEQ ID NO: 521 - protein, 34 aa, synthetic construct
  REGION 1..34: note = SYNTHETIC CONSTRUCT DERIVED FROM HUMAN THOUGHT
MKQIEDKIEEILSKIYHIENEIARIKKLIGERAG

SEQ ID NO: 522 - protein, 31 aa, synthetic construct
  REGION 1..31: note = SYNTHETIC CONSTRUCT DERIVED FROM HUMAN THOUGHT
LSSIEKKLEEITSQLIQISNELTLIRNELAQ

SEQ ID NO: 523 - protein, 31 aa, synthetic construct
  REGION 1..31: note = SYNTHETIC CONSTRUCT DERIVED FROM HUMAN THOUGHT
LSSIEKKLEEITSQLQQIRNELTLIRNELAQ

SEQ ID NO: 524 - protein, 31 aa, synthetic construct
  REGION 1..31: note = SYNTHETIC CONSTRUCT DERIVED FROM HUMAN THOUGHT
LSSLEKKLEELTSQLIQLRNELTLLRNELAQ

SEQ ID NO: 525 - protein, 31 aa, synthetic construct
  REGION 1..31: note = SYNTHETIC CONSTRUCT DERIVED FROM HUMAN THOUGHT
ISSLEKKIEELTSQIQQLRNEITLLRNEIAQ

SEQ ID NO: 526 - protein, 47 aa, synthetic construct; identical to SEQ ID NO: 518
  REGION 1..47: note = SYNTHETIC CONSTRUCT DERIVED FROM HUMAN THOUGHT

SEQ ID NO: 527 - protein, 64 aa, synthetic construct
  REGION 1..64: note = SYNTHETIC CONSTRUCT DERIVED FROM HUMAN THOUGHT
VTAFSNMDDMLQKAHLVIEGTFIYLRDSTEFFIRVRDGWKKLQLGELIPIPADSPPPPALSSNP

SEQ ID NO: 528 - protein, 31 aa, synthetic construct
  REGION 1..31: note = SYNTHETIC CONSTRUCT DERIVED FROM HUMAN THOUGHT
LKSVENRLAVVENQLKTVIEELKTVKDLLSN

SEQ ID NO: 529 - protein, 31 aa, synthetic construct
  REGION 1..31: note = SYNTHETIC CONSTRUCT DERIVED FROM HUMAN THOUGHT
LARIEEKLKTIKAQLSEIASTLNMIREQLAQ

SEQ ID NO: 530 - protein, 31 aa, synthetic construct
  REGION 1..31: note = SYNTHETIC CONSTRUCT DERIVED FROM HUMAN THOUGHT
VSRLEEKVKTLKSQVTELASTVSLLREQVAQ

SEQ ID NO: 531 - protein, 30 aa, synthetic construct
  REGION 1..30: note = SYNTHETIC CONSTRUCT DERIVED FROM HUMAN THOUGHT
IQSEKKIEDISSLIGQIQSEITLIRNEIAQ

SEQ ID NO: 532 - protein, 31 aa, synthetic construct
  REGION 1..31: note = SYNTHETIC CONSTRUCT DERIVED FROM HUMAN THOUGHT
LMSLEKKLEELTQTLMQLQNELSMLKNELAQ

SEQ ID NO: 533 - protein, 157 aa, Homo sapiens
VRSSSRTPSDKPVAHVVANPQAEGQLQWLNRRANALLANGVELRDNQLVVPSEGLYLIYSQVLFKGQGCPSTHVLLTHTISRIAVSSQTKVNLLSAIKSPCQRETPEGAEAKPWYEPIYLGGVFQLEKGDRLSAEINRPDYLDFAESGQVYFGIIAL

SEQ ID NO: 534 - protein, 40 aa, synthetic construct
  REGION 1..40: note = SYNTHETIC CONSTRUCT DERIVED FROM HUMAN THOUGHT
SHGFPPEVEEQDDGTLPMSCAQESGMDRHPAACASARINV

SEQ ID NO: 535 - protein, 17 aa, synthetic construct
  REGION 1..17: note = SYNTHETIC CONSTRUCT DERIVED FROM HUMAN THOUGHT
HNPSERLPLAQVSAHPW

SEQ ID NO: 536 - protein, 17 aa, synthetic construct
  REGION 1..17: note = SYNTHETIC CONSTRUCT DERIVED FROM HUMAN THOUGHT
LCSKLRNLLHDNELSDL

SEQ ID NO: 537 - protein, 17 aa, synthetic construct
  REGION 1..17: note = SYNTHETIC CONSTRUCT DERIVED FROM HUMAN THOUGHT
AWAHSRAALDRLEKLLR

SEQ ID NO: 538 - protein, 17 aa, synthetic construct
  REGION 1..17: note = SYNTHETIC CONSTRUCT DERIVED FROM HUMAN THOUGHT
FIFMRRSSLLSRSSSGY

SEQ ID NO: 539 - protein, 17 aa, synthetic construct
  REGION 1..17: note = SYNTHETIC CONSTRUCT DERIVED FROM HUMAN THOUGHT
GPFATRSPLFIFMRRSS

SEQ ID NO: 540 - protein, 17 aa, synthetic construct
  REGION 1..17: note = SYNTHETIC CONSTRUCT DERIVED FROM HUMAN THOUGHT
SSVPQRSTLAELKSKGK

SEQ ID NO: 541 - protein, 15 aa, synthetic construct
  REGION 1..15: note = SYNTHETIC CONSTRUCT DERIVED FROM HUMAN THOUGHT
QQPRTRAALAVLKSG

SEQ ID NO: 542 - protein, 17 aa, synthetic construct
  REGION 1..17: note = SYNTHETIC CONSTRUCT DERIVED FROM HUMAN THOUGHT
PGLRPRTALGDIGNKVS

SEQ ID NO: 543 - protein, 17 aa, synthetic construct
  REGION 1..17: note = SYNTHETIC CONSTRUCT DERIVED FROM HUMAN THOUGHT
GALKKRSAFEDLTNASQ

SEQ ID NO: 544 - protein, 17 aa, synthetic construct
  REGION 1..17: note = SYNTHETIC CONSTRUCT DERIVED FROM HUMAN THOUGHT
LPLSPRKRLGDDNLCNT

SEQ ID NO: 545 - protein, 17 aa, synthetic construct
  REGION 1..17: note = SYNTHETIC CONSTRUCT DERIVED FROM HUMAN THOUGHT
SKVLGRSPLTILQDDNS

SEQ ID NO: 546 - protein, 17 aa, synthetic construct
  REGION 1..17: note = SYNTHETIC CONSTRUCT DERIVED FROM HUMAN THOUGHT
GPRRGRDELGGGRRPGT

SEQ ID NO: 547 - protein, 17 aa, synthetic construct
  REGION 1..17: note = SYNTHETIC CONSTRUCT DERIVED FROM HUMAN THOUGHT
TKIAHRKSLSQKENRHK

SEQ ID NO: 548 - protein, 17 aa, synthetic construct
  REGION 1..17: note = SYNTHETIC CONSTRUCT DERIVED FROM HUMAN THOUGHT
VTCLLRKRLPVTNEMVH

SEQ ID NO: 549 - protein, 17 aa, synthetic construct
  REGION 1..17: note = SYNTHETIC CONSTRUCT DERIVED FROM HUMAN THOUGHT
LEIRDRMWLKITIANAV

SEQ ID NO: 550 - protein, 17 aa, synthetic construct
  REGION 1..17: note = SYNTHETIC CONSTRUCT DERIVED FROM HUMAN THOUGHT
VANSYRGTLKRQPAFDT

SEQ ID NO: 551 - protein, 17 aa, synthetic construct
  REGION 1..17: note = SYNTHETIC CONSTRUCT DERIVED FROM HUMAN THOUGHT
RQFQRRLPLRAVNLRAG

SEQ ID NO: 552 - protein, 17 aa, synthetic construct
  REGION 1..17: note = SYNTHETIC CONSTRUCT DERIVED FROM HUMAN THOUGHT
DKKRPRRSLSISKNKKK

SEQ ID NO: 553 - protein, 17 aa, synthetic construct
  REGION 1..17: note = SYNTHETIC CONSTRUCT DERIVED FROM HUMAN THOUGHT
EYVMFRTPLASVQKSAA

SEQ ID NO: 554 - protein, 16 aa, synthetic construct
  REGION 1..16: note = SYNTHETIC CONSTRUCT DERIVED FROM HUMAN THOUGHT
EYVMFRTPLASVQKSA

SEQ ID NO: 555 - protein, 17 aa, synthetic construct
  REGION 1..17: note = SYNTHETIC CONSTRUCT DERIVED FROM HUMAN THOUGHT
NSSVPRRTLKMIQPSAS

SEQ ID NO: 556 - protein, 17 aa, synthetic construct
  REGION 1..17: note = SYNTHETIC CONSTRUCT DERIVED FROM HUMAN THOUGHT
FVLQLRPELQDALISII

SEQ ID NO: 557 - protein, 17 aa, synthetic construct
  REGION 1..17: note = SYNTHETIC CONSTRUCT DERIVED FROM HUMAN THOUGHT
EHAAFRFALSQLTEPPK

SEQ ID NO: 558 - protein, 17 aa, synthetic construct
  REGION 1..17: note = SYNTHETIC CONSTRUCT DERIVED FROM HUMAN THOUGHT
EVWQRRGSLEGAAAAAE

SEQ ID NO: 559 - protein, 17 aa, synthetic construct
  REGION 1..17: note = SYNTHETIC CONSTRUCT DERIVED FROM HUMAN THOUGHT
ENFALKTPLKEGNTNCY

SEQ ID NO: 560 - protein, 17 aa, synthetic construct
  REGION 1..17: note = SYNTHETIC CONSTRUCT DERIVED FROM HUMAN THOUGHT
EVSKLRCQLAKKKQSET

SEQ ID NO: 561 - protein, 17 aa, synthetic construct
  REGION 1..17: note = SYNTHETIC CONSTRUCT DERIVED FROM HUMAN THOUGHT
WFQNRRMKLKKMNRENR

SEQ ID NO: 562 - protein, 17 aa, synthetic construct
  REGION 1..17: note = SYNTHETIC CONSTRUCT DERIVED FROM HUMAN THOUGHT
APFEQRASLNPRAEHLE

SEQ ID NO: 563 - protein, 17 aa, synthetic construct
  REGION 1..17: note = SYNTHETIC CONSTRUCT DERIVED FROM HUMAN THOUGHT
SLLKERKFLSLASNPEL

SEQ ID NO: 564 - protein, 17 aa, synthetic construct
  REGION 1..17: note = SYNTHETIC CONSTRUCT DERIVED FROM HUMAN THOUGHT
LDPSNRKPLTVLNKGLE

SEQ ID NO: 565 - protein, 17 aa, synthetic construct
  REGION 1..17: note = SYNTHETIC CONSTRUCT DERIVED FROM HUMAN THOUGHT
LPKATRKALGTVNRATE

SEQ ID NO: 566 - protein, 17 aa, synthetic construct
  REGION 1..17: note = SYNTHETIC CONSTRUCT DERIVED FROM HUMAN THOUGHT
VGGEKRLCLPQVLNSVL

SEQ ID NO: 567 - protein, 14 aa, synthetic construct
  REGION 1..14: note = SYNTHETIC CONSTRUCT DERIVED FROM HUMAN THOUGHT
MHRKHLQEIPDLSS

SEQ ID NO: 568 - protein, 13 aa, synthetic construct
  REGION 1..13: note = SYNTHETIC CONSTRUCT DERIVED FROM HUMAN THOUGHT
GGEAQKENGIYSV

SEQ ID NO: 569 - protein, 13 aa, synthetic construct
  REGION 1..13: note = SYNTHETIC SEQUENCE DERIVED FROM HUMAN THOUGHT
HILEDKENVVAKQ

SEQ ID NO: 570 - protein, 13 aa, synthetic construct
  REGION 1..13: note = SYNTHETIC CONSTRUCT DERIVED FROM HUMAN THOUGHT
FEDGNKENYGLPQ

SEQ ID NO: 571 - protein, 13 aa, synthetic construct
  REGION 1..13: note = SYNTHETIC CONSTRUCT DERIVED FROM HUMAN THOUGHT
PMPRAKENELQAG

SEQ ID NO: 572 - protein, 13 aa, synthetic construct
  REGION 1..13: note = SYNTHETIC CONSTRUCT DERIVED FROM HUMAN THOUGHT
EWELSKENVQPLR

SEQ ID NO: 573 - protein, 13 aa, synthetic construct
  REGION 1..13: note = SYNTHETIC CONSTRUCT DERIVED FROM HUMAN THOUGHT
SFLLSKENQPENS

SEQ ID NO: 574 - protein, 13 aa, synthetic construct
  REGION 1..13: note = SYNTHETIC CONSTRUCT DERIVED FROM HUMAN THOUGHT
DPDENKENEAFEF

SEQ ID NO: 575 - protein, 13 aa, synthetic construct
  REGION 1..13: note = SYNTHETIC CONSTRUCT DERIVED FROM HUMAN THOUGHT
SSGEDKENDGFVF

SEQ ID NO: 576 - protein, 13 aa, synthetic construct
  REGION 1..13: note = SYNTHETIC CONSTRUCT DERIVED FROM HUMAN THOUGHT
SSSANKENDNGNL

SEQ ID NO: 577 - protein, 13 aa, synthetic construct
  REGION 1..13: note = SYNTHETIC CONSTRUCT DERIVED FROM HUMAN THOUGHT
PKQGKKENGPPHS

SEQ ID NO: 578 - protein, 13 aa, synthetic construct
  REGION 1..13: note = SYNTHETIC CONSTRUCT DERIVED FROM HUMAN THOUGHT
ENSSQKENVFKCD

SEQ ID NO: 579 - protein, 13 aa, synthetic construct
  REGION 1..13: note = SYNTHETIC CONSTRUCT DERIVED FROM HUMAN THOUGHT
QEMEEKENCHLEQ

SEQ ID NO: 580 - protein, 13 aa, synthetic construct
  REGION 1..13: note = SYNTHETIC CONSTRUCT DERIVED FROM HUMAN THOUGHT
DVILLKENSTAFE

SEQ ID NO: 581 - protein, 13 aa, synthetic construct
  REGION 1..13: note = SYNTHETIC CONSTRUCT DERIVED FROM HUMAN THOUGHT
SHGKDKENRGINT

SEQ ID NO: 582 - protein, 13 aa, synthetic construct
  REGION 1..13: note = SYNTHETIC CONSTRUCT DERIVED FROM HUMAN THOUGHT
QKAEEKENHCPTM

SEQ ID NO: 583 - protein, 13 aa, synthetic construct
  REGION 1..13: note = SYNTHETIC CONSTRUCT DERIVED FROM HUMAN THOUGHT
FSGESKENIMRSE

SEQ ID NO: 584 - protein, 13 aa, synthetic construct
  REGION 1..13: note = SYNTHETIC CONSTRUCT DERIVED FROM HUMAN THOUGHT
LTLALKENSRLQK

SEQ ID NO: 585 - protein, 13 aa, synthetic construct
  REGION 1..13: note = SYNTHETIC CONSTRUCT DERIVED FROM HUMAN THOUGHT
PWGQSKENNYLNQ

SEQ ID NO: 586 - protein, 13 aa, synthetic construct
  REGION 1..13: note = SYNTHETIC CONSTRUCT DERIVED FROM HUMAN THOUGHT
PKDSEKENQIPEE

SEQ ID NO: 587 - protein, 13 aa, synthetic construct
  REGION 1..13: note = SYNTHETIC CONSTRUCT DERIVED FROM HUMAN THOUGHT
ILHFAKENDFKAF

SEQ ID NO: 588 - protein, 13 aa, synthetic construct
  REGION 1..13: note = SYNTHETIC CONSTRUCT DERIVED FROM HUMAN THOUGHT
TQHPEKENEGDIT

SEQ ID NO: 589 - protein, 13 aa, synthetic construct
  REGION 1..13: note = SYNTHETIC CONSTRUCT DERIVED FROM HUMAN THOUGHT
AGPDNKENCPVPG

SEQ ID NO: 590 - protein, 16 aa, synthetic construct
  REGION 1..16: note = SYNTHETIC CONSTRUCT DERIVED FROM HUMAN THOUGHT
SLSSAFHVFEDGNKEN

SEQ ID NO: 591 - protein, 16 aa, synthetic construct
  REGION 1..16: note = SYNTHETIC CONSTRUCT DERIVED FROM HUMAN THOUGHT
GPSVPFSIFDEFLLSE

SEQ ID NO: 592 - protein, 16 aa, synthetic construct
  REGION 1..16: note = SYNTHETIC CONSTRUCT DERIVED FROM HUMAN THOUGHT
QNNSRITVFDENADEA

SEQ ID NO: 593 - protein, 16 aa, synthetic construct
  REGION 1..16: note = SYNTHETIC CONSTRUCT DERIVED FROM HUMAN THOUGHT
AVLPSFTPYVEETARQ

SEQ ID NO: 594 - protein, 16 aa, synthetic construct
  REGION 1..16: note = SYNTHETIC CONSTRUCT DERIVED FROM HUMAN THOUGHT
SKQPAFTIHVDEAEKE

SEQ ID NO: 595 - protein, 7 aa, synthetic construct
  REGION 1..7: note = SYNTHETIC CONSTRUCT DERIVED FROM HUMAN THOUGHT
ISKGNLR

SEQ ID NO: 596 - protein, 7 aa, synthetic construct
  REGION 1..7: note = SYNTHETIC CONSTRUCT DERIVED FROM HUMAN THOUGHT
RQILGMR

SEQ ID NO: 597 - protein, 12 aa, synthetic construct
  REGION 1..12: note = SYNTHETIC CONSTRUCT DERIVED FROM HUMAN THOUGHT
GTKKSKKNLRRL

SEQ ID NO: 598 - protein, 4 aa, synthetic construct
  REGION 1..4: note = SYNTHETIC CONSTRUCT DERIVED FROM HUMAN THOUGHT
HDEF

SEQ ID NO: 599 - protein, 4 aa, synthetic construct
  REGION 1..4: note = SYNTHETIC CONSTRUCT DERIVED FROM HUMAN THOUGHT
HDEL

SEQ ID NO: 600 - protein, 4 aa, synthetic construct
  REGION 1..4: note = SYNTHETIC CONSTRUCT DERIVED FROM HUMAN THOUGHT
RDEF

SEQ ID NO: 601 - protein, 4 aa, synthetic construct
  REGION 1..4: note = SYNTHETIC CONSTRUCT DERIVED FROM HUMAN THOUGHT
RDEL

SEQ ID NO: 602 - protein, 4 aa, synthetic construct
  REGION 1..4: note = SYNTHETIC CONSTRUCT DERIVED FROM HUMAN THOUGHT
WDEL

SEQ ID NO: 603 - protein, 4 aa, synthetic construct
  REGION 1..4: note = SYNTHETIC CONSTRUCT DERIVED FROM HUMAN THOUGHT
YDEL

SEQ ID NO: 604 - protein, 4 aa, synthetic construct
  REGION 1..4: note = SYNTHETIC CONSTRUCT DERIVED FROM HUMAN THOUGHT
HEEF

SEQ ID NO: 605 - protein, 4 aa, synthetic construct
  REGION 1..4: note = SYNTHETIC CONSTRUCT DERIVED FROM HUMAN THOUGHT
HEEL

SEQ ID NO: 606 - protein, 4 aa, synthetic construct
  REGION 1..4: note = SYNTHETIC CONSTRUCT DERIVED FROM HUMAN THOUGHT
KEEL

SEQ ID NO: 607 - protein, 4 aa, synthetic construct
  REGION 1..4: note = SYNTHETIC CONSTRUCT DERIVED FROM HUMAN THOUGHT
REEL

SEQ ID NO: 608 - protein, 4 aa, synthetic construct
  REGION 1..4: note = SYNTHETIC CONSTRUCT DERIVED FROM HUMAN THOUGHT
KAEL

SEQ ID NO: 609 - protein, 4 aa, synthetic construct
  REGION 1..4: note = SYNTHETIC CONSTRUCT DERIVED FROM HUMAN THOUGHT
KCEL

SEQ ID NO: 610 - protein, 4 aa, synthetic construct
  REGION 1..4: note = SYNTHETIC CONSTRUCT DERIVED FROM HUMAN THOUGHT
KFEL

SEQ ID NO: 611 - protein, 4 aa, synthetic construct
  REGION 1..4: note = SYNTHETIC CONSTRUCT DERIVED FROM HUMAN THOUGHT
KGEL

SEQ ID NO: 612 - protein, 4 aa, synthetic construct
  REGION 1..4: note = SYNTHETIC CONSTRUCT DERIVED FROM HUMAN THOUGHT
KHEL

SEQ ID NO: 613 - protein, 4 aa, synthetic construct
  REGION 1..4: note = SYNTHETIC CONSTRUCT DERIVED FROM HUMAN THOUGHT
KLEL

SEQ ID NO: 614 - protein, 4 aa, synthetic construct
  REGION 1..4: note = SYNTHETIC CONSTRUCT DERIVED FROM HUMAN THOUGHT
KNEL

SEQ ID NO: 615 - protein, 4 aa, synthetic construct
  REGION 1..4: note = SYNTHETIC CONSTRUCT DERIVED FROM HUMAN THOUGHT
KQEL

SEQ ID NO: 616 - protein, 4 aa, synthetic construct
  REGION 1..4: note = SYNTHETIC CONSTRUCT DERIVED FROM HUMAN THOUGHT
KREL

SEQ ID NO: 617 - protein, 4 aa, synthetic construct
  REGION 1..4: note = SYNTHETIC CONSTRUCT DERIVED FROM HUMAN THOUGHT
KSEL

SEQ ID NO: 618 - protein, 4 aa, synthetic construct
  REGION 1..4: note = SYNTHETIC CONSTRUCT DERIVED FROM HUMAN THOUGHT
KVEL

SEQ ID NO: 619 - protein, 4 aa, synthetic construct
  REGION 1..4: note = SYNTHETIC CONSTRUCT DERIVED FROM HUMAN THOUGHT
KWEL

SEQ ID NO: 620 - protein, 4 aa, synthetic construct
  REGION 1..4: note = SYNTHETIC CONSTRUCT DERIVED FROM HUMAN THOUGHT
KYEL

SEQ ID NO: 621 - protein, 4 aa, synthetic construct
  REGION 1..4: note = SYNTHETIC CONSTRUCT DERIVED FROM HUMAN THOUGHT
KEDL

SEQ ID NO: 622 - protein, 4 aa, synthetic construct
  REGION 1..4: note = SYNTHETIC CONSTRUCT DERIVED FROM HUMAN THOUGHT
KIEL

SEQ ID NO: 623 - protein, 4 aa, synthetic construct
  REGION 1..4: note = SYNTHETIC CONSTRUCT DERIVED FROM HUMAN THOUGHT
DKEL

SEQ ID NO: 624 - protein, 4 aa, synthetic construct
  REGION 1..4: note = SYNTHETIC CONSTRUCT DERIVED FROM HUMAN THOUGHT
FDEL

SEQ ID NO: 625 - protein, 4 aa, synthetic construct
  REGION 1..4: note = SYNTHETIC CONSTRUCT DERIVED FROM HUMAN THOUGHT
KDEF

SEQ ID NO: 626 - protein, 4 aa, synthetic construct
  REGION 1..4: note = SYNTHETIC CONSTRUCT DERIVED FROM HUMAN THOUGHT
KKEL

SEQ ID NO: 627 - protein, 4 aa, synthetic construct
  REGION 1..4: note = SYNTHETIC CONSTRUCT DERIVED FROM HUMAN THOUGHT
HADL

SEQ ID NO: 628 - protein, 4 aa, synthetic construct
  REGION 1..4: note = SYNTHETIC CONSTRUCT DERIVED FROM HUMAN THOUGHT
HAEL

SEQ ID NO: 629 - protein, 4 aa, synthetic construct
  REGION 1..4: note = SYNTHETIC CONSTRUCT DERIVED FROM HUMAN THOUGHT
HIEL

SEQ ID NO: 630 - protein, 4 aa, synthetic construct
  REGION 1..4: note = SYNTHETIC CONSTRUCT DERIVED FROM HUMAN THOUGHT
HNEL

SEQ ID NO: 631 - protein, 4 aa, synthetic construct
  REGION 1..4: note = SYNTHETIC CONSTRUCT DERIVED FROM HUMAN THOUGHT
HTEL

SEQ ID NO: 632 - protein, 4 aa, synthetic construct
  REGION 1..4: note = SYNTHETIC CONSTRUCT DERIVED FROM HUMAN THOUGHT
KTEL

SEQ ID NO: 633 - protein, 4 aa, synthetic construct
  REGION 1..4: note = SYNTHETIC CONSTRUCT DERIVED FROM HUMAN THOUGHT
HVEL

SEQ ID NO: 634 - protein, 4 aa, synthetic construct
  REGION 1..4: note = SYNTHETIC CONSTRUCT DERIVED FROM HUMAN THOUGHT
NDEL

SEQ ID NO: 635 - protein, 4 aa, synthetic construct
  REGION 1..4: note = SYNTHETIC CONSTRUCT DERIVED FROM HUMAN THOUGHT
QDEL

SEQ ID NO: 636 - protein, 4 aa, synthetic construct
  REGION 1..4: note = SYNTHETIC CONSTRUCT DERIVED FROM HUMAN THOUGHT
REDL

SEQ ID NO: 637 - protein, 4 aa, synthetic construct
  REGION 1..4: note = SYNTHETIC CONSTRUCT DERIVED FROM HUMAN THOUGHT
RNEL

SEQ ID NO: 638 - protein, 4 aa, synthetic construct
  REGION 1..4: note = SYNTHETIC CONSTRUCT DERIVED FROM HUMAN THOUGHT
RTDL

SEQ ID NO: 639 - protein, 4 aa, synthetic construct
  REGION 1..4: note = SYNTHETIC CONSTRUCT DERIVED FROM HUMAN THOUGHT
RTEL

SEQ ID NO: 640 - protein, 4 aa, synthetic construct
  REGION 1..4: note = SYNTHETIC CONSTRUCT DERIVED FROM HUMAN THOUGHT
SDEL

SEQ ID NO: 641 - protein, 4 aa, synthetic construct
  REGION 1..4: note = SYNTHETIC CONSTRUCT DERIVED FROM HUMAN THOUGHT
TDEL

SEQ ID NO: 642 - protein, 4 aa, synthetic construct
  REGION 1..4: note = SYNTHETIC CONSTRUCT DERIVED FROM HUMAN THOUGHT
SKEL

SEQ ID NO: 643 - DNA, 52 bp, synthetic construct
  misc_feature 1..52: note = SYNTHETIC CONSTRUCT DERIVED FROM HUMAN THOUGHT
  source 1..52: mol_type = other DNA
agctatgaggtacatgattttaggcttgctcgcccttgcggcagctgcaggc

SEQ ID NO: 644 - protein, 17 aa, Human mastadenovirus C
RYMILGLLALAAVCSAA

SEQ ID NO: 645 - protein, 18 aa, Human mastadenovirus C
MLLPVPLLLGLLGLAAAL

SEQ ID NO: 646 - protein, 15 aa, Bovine alphaherpesvirus 1
DDELGLIMAAPARLV

SEQ ID NO: 647 - protein, 15 aa, synthetic construct
  REGION 1..15: note = SYNTHETIC CONSTRUCT DERIVED FROM HUMAN THOUGHT
DDELGLPMAAPARLV

SEQ ID NO: 648 - protein, 15 aa, synthetic construct
  REGION 1..15: note = SYNTHETIC CONSTRUCT DERIVED FROM HUMAN THOUGHT
DDELGLGMAAPARLV

SEQ ID NO: 649 - protein, 15 aa, synthetic construct
  REGION 1..15: note = SYNTHETIC CONSTRUCT DERIVED FROM HUMAN THOUGHT
DDELGLDMAAPARLV

SEQ ID NO: 650 - protein, 15 aa, Mus musculus
QGLVVHLSNPIMRNN

SEQ ID NO: 651 - protein, 15 aa, synthetic construct
  REGION 1..15: note = SYNTHETIC CONSTRUCT DERIVED FROM HUMAN THOUGHT
QGLVDHLSNPIMRNN

SEQ ID NO: 652 - protein, 15 aa, synthetic construct
  REGION 1..15: note = SYNTHETIC CONSTRUCT DERIVED FROM HUMAN THOUGHT
QGLVVHLSNPDMRNN

SEQ ID NO: 653 - protein, 15 aa, synthetic construct
  REGION 1..15: note = SYNTHETIC CONSTRUCT DERIVED FROM HUMAN THOUGHT
QGLVVHDSNPIMRNN

SEQ ID NO: 654 - protein, 42 aa, Porcine circovirus 2
MWLGSASSILLAGHVAAEVLPRCCRCRSALVILTAHFFRFQL

SEQ ID NO: 655 - protein, 314 aa, synthetic construct
  REGION 1..314: note = SYNTHETIC CONSTRUCT DERIVED FROM HUMAN THOUGHT
MPSKKNGRSGPQPHKRWVFTLNNPSEDERKKIRELPISLFDYFIVGEEGNEEGRTPHLQGFANFVKKQTFNKVKWYLGARCYIEKAKGTDQQNKEYCSKEGNLLIECGAPRSQGQRSDLSTAVSTLLESGSLVTVAEQHPVTFVRNFRGLAELLKVSGKMQKRDWKTNVHVIVGPPGCGKSKWAANFADPETTYWKPPRNKWWDGYHGEEVVVIDDFYGWLPWDDLLRLCDRYPLTVETKGGTVPFLARSILITSNQTPLEWYSSTAVPAVEALYRRITSLVFWKNATEQSTEEGGQFVTLSPPCPEFPYEINY

SEQ ID NO: 656 - protein, 314 aa, synthetic construct
  REGION 1..314: note = SYNTHETIC CONSTRUCT DERIVED FROM HUMAN THOUGHT
MPSKKNGRSGPQPHKRWVFTLNNPSEDERKKIRELPISLFDYFIVGEEGNEEGRTPHLQGFANFTKKQTFNKVKWYLGARCYIEKAKGTDQQNKEYCSKEGNLLIECGAPRSQGQRSDLSTAVSTLLESGSLVTVAEQHPVTFVRNFRGLAELLKVSGKMQKRDWKTNVHVIVGPPGCGKSKWAANFADPETTYWKPPRNKWWDGYHGEEVVVIDDFYGWLPWDDLLRLCDRYPLTVETKGGTVPFLARSILITSNQTPLEWYSSTAVPAVEALYRRITSLVFWKDATEQSTEEGGQFVTLSPPCPEFPYEINY

SEQ ID NO: 657 - protein, 314 aa, synthetic construct
  REGION 1..314: note = SYNTHETIC CONSTRUCT DERIVED FROM HUMAN THOUGHT
MPSKKNGRSGPQPHKRWVFTLNNPSEDERKKIRELPISLFDYFIVGEEGNEEGRTPHLQGFANFTKKQTFNKVKWYLGARCYIEKAKGTDQQNKEYCSKEGNLLIECGAPRSQGQRSDLSTAVSTLLESGSLVTVAEQHPVTFVRNFRGLAELLKVSGKMQKRDWKTNVHVIVGPPGCGKSKWAANFADPETTYWKPPRNKWWDGYHGEEVVVIDDFYGWLPWDDLLRLCDRYPLTVETKGGTVPFLARSILITSDQTPLEWYSSTAVPAVEALYRRITSLVFWKDATEQSTEEGGQFVTLSPPCPEFPYEINY

SEQ ID NO: 658 - protein, 104 aa, Porcine circovirus 2
MVTIPPLVSRWFPVCGFRVCKISSPFAFTTPRWPHNDVYIGLPITLLHFPAHFQKFSQPAEISDKRYRVLLCNGHQTPALQQGTHSSRQVTPLSLRSRSSTFNK

SEQ ID NO: 659 - protein, 59 aa, Porcine circovirus 2
MTCTLVFQSRFCIFPLTFKSSASPRKFLTNVTGCCSATVTRLPLSNKVLTAVDRSLRCP

SEQ ID NO: 660 - protein, 60 aa, synthetic construct
  REGION 1..60: note = SYNTHETIC CONSTRUCT DERIVED FROM HUMAN THOUGHT
MTCTLVFQSRFCIFPLTFKSSASPRKFLTDVTGCCSATVTRLPLSNKVLTAVDRSLRCPS

SEQ ID NO: 661 - protein, 21 aa, Porcine circovirus 2
MDIDHTVSVDHPTAASHKSHQ

SEQ ID NO: 662 - protein, 14 aa, Porcine circovirus 2
MKNKNHYEVIKKTQ

SEQ ID NO: 663 - protein, 53 aa, Porcine circovirus 2
MVFIFHLGFKWGVFKIKFSELYIHGYTDIVVLVVYTVFERSAEAYVVHISRGL

SEQ ID NO: 664 - protein, 29 aa, Porcine circovirus 2
MASSTPASPAPSDILSRLPQSERPPGRWT

SEQ ID NO: 665 - protein, 19 aa, Porcine circovirus 2
MAAGAVSSSAVTPPWIRHS

SEQ ID NO: 666 - protein, 234 aa, synthetic construct
  REGION 1..234: note = SYNTHETIC CONSTRUCT DERIVED FROM HUMAN THOUGHT
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIRKVKVEFWPCSPITQGDRGVGSTAVILDDNFVTKATALTYDPYVDYSSRHTIPQPFSYHSRYFTPKPVLDSTIDYFQPNNKRTQLWLRLQTSRNVDHVGLGTAFENSIYDQDYNIRVTMYVQFREFNLKDPPLKPK

SEQ ID NO: 667 - protein, 235 aa, synthetic construct
  REGION 1..235: note = SYNTHETIC CONSTRUCT DERIVED FROM HUMAN THOUGHT
  VARIANT 8: note = MISC_FEATURE - Xaa8 IS Tyr OR Phe
  VARIANT 21: note = MISC_FEATURE - Xaa21 IS Gln, Leu, OR His
  VARIANT 47: note = MISC_FEATURE - Xaa47 IS Thr, Ala, OR Ser
  VARIANT 52: note = MISC_FEATURE - Xaa52 IS Thr OR Ser
  VARIANT 53: note = MISC_FEATURE - Xaa53 IS Phe OR Ile
  VARIANT 54: note = MISC_FEATURE - Xaa54 IS Gly OR Val
  VARIANT 57: note = MISC_FEATURE - Xaa57 IS Val OR Ile
  VARIANT 58: note = MISC_FEATURE - Xaa58 IS Lys OR Asn
  VARIANT 59: note = MISC_FEATURE - Xaa59 IS Ala OR Arg
  VARIANT 60: note = MISC_FEATURE - Xaa60 IS Thr OR Ser
  VARIANT 61: note = MISC_FEATURE - Xaa61 IS Thr OR Gln
  VARIANT 63: note = MISC_FEATURE - Xaa63 IS Arg, Ser, OR Thr
  VARIANT 75: note = MISC_FEATURE - Xaa75 IS Asp, Asn, OR Lys
  VARIANT 76: note = MISC_FEATURE - Xaa76 IS Ile OR Phe
  VARIANT 77: note = MISC_FEATURE - Xaa77 IS Asp OR Asn
  VARIANT 78: note = MISC_FEATURE - Xaa78 IS Asp OR Gln
  VARIANT 86: note = MISC_FEATURE - Xaa86 IS Thr OR Ser
  VARIANT 88: note = MISC_FEATURE - Xaa88 IS Lys OR Pro
  VARIANT 89: note = MISC_FEATURE - Xaa89 IS Ile, Arg, OR Leu
  VARIANT 90: note = MISC_FEATURE - Xaa90 IS Ser OR Thr
  VARIANT 106: note = MISC_FEATURE - Xaa106 IS Trp OR Arg
  VARIANT 107: note = Xaa107 IS Pro OR Ala
  VARIANT 108: note = MISC_FEATURE - Xaa108 IS Cys OR Arg
  VARIANT 126: note = MISC_FEATURE - Xaa126 IS Thr OR Ser
  VARIANT 143: note = MISC_FEATURE - Xaa143 IS Asp OR Asn
  VARIANT 189: note = misc_feature - Xaa189 can be any naturally occurring amino acid
  VARIANT 190: note = Xaa190 IS Ser, Ala, OR Thr
  VARIANT 191: note = MISC_FEATURE - Xaa191 IS Arg, Gly, OR Lys
  VARIANT 199: note = misc_feature - Xaa199 can be any naturally occurring amino acid
  VARIANT 200: note = MISC_FEATURE - Xaa200 IS Thr OR His
  VARIANT 202: note = misc_feature - Xaa202 can be any naturally occurring amino acid
  VARIANT 203: note = MISC_FEATURE - Xaa203 IS Glu OR Gln
  VARIANT 205: note = misc_feature - Xaa205 can be any naturally occurring amino acid
  VARIANT 206: note = Xaa206 IS Ile, Thr, OR Lys
  VARIANT 207: note = MISC_FEATURE - Xaa207 IS Tyr OR Asn
  VARIANT 208: note = MISC_FEATURE - Xaa208 IS Asp OR Ala
  VARIANT 209: note = misc_feature - Xaa209 can be any naturally occurring amino acid
  VARIANT 210: note = MISC_FEATURE - Xaa210 IS Asp, Glu, OR Ala
  VARIANT 212: note = Xaa212 can be any naturally occurring amino acid
  VARIANT 213: note = MISC_FEATURE - Xaa213 IS Ile OR Val
  VARIANT 214: note = misc_feature - Xaa214 can be any naturally occurring amino acid
  VARIANT 215: note = MISC_FEATURE - Xaa215 IS Ile OR Val
  VARIANT 231: note = misc_feature - Xaa231 can be any naturally occurring amino acid
  VARIANT 232: note = MISC_FEATURE - Xaa232 IS Lys OR Asn
  VARIANT 233: note = misc_feature - Xaa233 can be any naturally occurring amino acid
  VARIANT 234: note = MISC_FEATURE - Xaa234 IS Pro, Lys, OR IS ABSENT
  VARIANT 235: note = MISC_FEATURE - XAA235 IS Lys OR IS ABSENT
MTYPRRRXRRRRHRPRSHLGXILRRRPWLVHPRHRYRWRRKNGIFNXRLSRXXXYTXXXXXVXTPSWAVDMMRFXXXXFVPPGGGXNXXXIPFEYYRIRKVKVEFXXXSPITQGDRGVGSTAVILXDNFVTKATALTYDPYVXYSSRHTIPQPFSYHSRYFTPKPVLDSTIDYFQPNNKRNLWLRLQTXXNVDHVGLGXAFXNSXXXQXYNXRXTMYVQFREFNLKDPPLXXXXX

SEQ ID NO: 668 - protein, 17 aa, Porcine reproductive and respiratory syndrome virus
RRGDAPEIGLPYKLYPV

SEQ ID NO: 669 - protein, 12 aa, Porcine reproductive and respiratory syndrome virus
QAAAEAYEPGRS

SEQ ID NO: 670 - protein, 9 aa, synthetic construct
  REGION 1..9: note = SYNTHETIC CONSTRUCT DERIVED FROM HUMAN THOUGHT
SDQISLYAQ

SEQ ID NO: 671 - protein, 4 aa, synthetic construct
  REGION 1..4: note = SYNTHETIC CONSTRUCT DERIVED FROM HUMAN THOUGHT
KYKY

SEQ ID NO: 672 - protein, 20 aa, Influenza A
NAELLVLMENERTLDFHDSN

SEQ ID NO: 673 - protein, 36 aa, Influenza A
QDLEKYVEDTKIDLWSYNAELLVALENQHTIDLTDS

SEQ ID NO: 674 - protein, 15 aa, Influenza A
KGILGFVFTLTVPSE

SEQ ID NO: 675 - protein, 40 aa, Influenza A
KTGGPIYRRRDGKWVRELILYDKEEIRRIWRQANNGEDAT

SEQ ID NO: 676 - protein, 23 aa, Influenza A
RLIQNSLTIERMVLSAFDERRNK

SEQ ID NO: 677 - protein, 18 aa, Influenza A
GKWVRELILYDKEEIRRI

SEQ ID NO: 678 - protein, 9 aa, Influenza A
VSDGGPNLY

SEQ ID NO: 679 - protein, 10 aa, Influenza A
RMVLASTTAK

SEQ ID NO: 680 - protein, 10 aa, Influenza A
ALASCMGLIY

SEQ ID NO: 681 - protein, 9 aa, Influenza A
MMMGMFNML

SEQ ID NO: 682 - protein, 20 aa, Influenza A
TYVLSIIPSGPLKAEIAQRL

SEQ ID NO: 683 - protein, 9 aa, Influenza A
SIIPSGPLK

SEQ ID NO: 684 - protein, 9 aa, Influenza A
ASNENMETM

SEQ ID NO: 685 - protein, 10 aa, Influenza A
SSLENFRAYV

SEQ ID NO: 686 - protein, 9 aa, Influenza A
ASNENMDAM

SEQ ID NO: 687 - protein, 12 aa, Influenza A
SFYRNVVWLIKK

SEQ ID NO: 688 - protein, 10 aa, Influenza A
SSLENFRAYV

SEQ ID NO: 689 - protein, 10 aa, Influenza A
SSAENFRAYV

SEQ ID NO: 690 - protein, 10 aa, Influenza A
SSLENFAAYV

SEQ ID NO: 691 - protein, 9 aa, Influenza A
LYIWGVHHP

SEQ ID NO: 692 - protein, 15 aa, Suid alphaherpesvirus 1
PVLFGEPFRAVCVVR

SEQ ID NO: 693 - protein, 20 aa, Suid alphaherpesvirus 1
SVRFVEGFAVCDGLCVPPEA

SEQ ID NO: 694 - protein, 385 aa, Suid alphaherpesvirus 1
ADVDAVPAPTFPPPAYPYTESWQLTLTTVPSPFVGPADVYHTRPLEDPCGVVALISDPQVDRLLNEAVAHRRPTYRAHVAWYRIADGCAHLLYFIEYADCDPRQVFGRCRRRTTPMWWTPSADYMFPTEDELGLLMVAPGRFNEGQYRRLVSVDGVNILTDFMVALPEGQECPFARVDQHRTYKFGACWSDDSFKRGVDVMRFLTPFYQQPPHREVVNYWYRKNGRTLPRAHAAATPYAIDPARPSAGSPRPRPRPRPRPRPKPEPAPATPAPPDRLPEPATRDHAAGGRPTPRPPRPETPHRPFAPPAVVPSGWPQPAEPFQPRTPAAPGVSRHRSVIVGTGTAMGALLVGVCVYIFFRLRGAKGYRLLGGPADADELKAQPGP

SEQ ID NO: 695 - protein, 338 aa, Suid alphaherpesvirus 1
ADVDAVPAPTFPPPAYPYTESWQLTLTTVPSPFVGPADVYHTRPLEDPCGVVALISDPQVDRLLNEAVAHRRPTYRAHVAWYRIADGCAHLLYFIEYADCDPRQVFGRCRRRTTPMWWTPSADYMFPTEDELGLLMVAPGRFNEGQYRRLVSVDGVNILTDFMVALPEGQECPFARVDQHRTYKFGACWSDDSFKRGVDVMRFLTPFYQQPPHREVVNYWYRKNGRTLPRAHAAATPYAIDPARPSAGSPRPRPRPRPRPRPKPEPAPATPAPPDRLPEPATRDHAAGGRPTPRPPRPETPHRPFAPPAVVPSGWPQPAEPFQPRTPAAPGVSRHRSV

SEQ ID NO: 696 - protein, 284 aa, Suid alphaherpesvirus 1
ADVDAVPAPTFPPPAYPYTESWQLTLTTVPSPFVGPADVYHTRPLEDPCGVVALISDPQVDRLLNEAVAHRRPTYRAHVAWYRIADGCAHLLYFIEYADCDPRQVFGRCRRRTTPMWWTPSADYMFPTEDELGLLMVAPGRFNEGQYRRLVSVDGVNILTDFMVALPEGQECPFARVDQHRTYKFGACWSDDSFKRGVDVMRFLTPFYQQPPHREVVNYWYRKNGRTLPRAHAAATPYAIDPARPSAGSPRPRPRPRPRPRPKPEPAPATPAPPDRLPEPATRD

SEQ ID NO: 697 - protein, 278 aa, African swine fever virus
MQIFVKTLTGKTITLEVEPSDTIENVKAKIQDKEGIPPDQQRLIFAGKQLEDGRTLSDYNIQKESTLHLVLRLRGAAADFILNISMKMEVIFKTDLRSSSQVVFHAGSLYNWFSVEIINSGRIVTTAIKTLLSTVKYDIVKSARIYAGQGYTEHQAQEEWNMILHVLFEEETESSASSENIHEKNDNETNECTSSFETLFEQEPSSEVPKDSKLYMLAQKTVQHIEQYGKAPDFNKVIRAHNFIQTIYGTPLKEEEKEVVRLMVIKLLKKISFYLTYI

SEQ ID NO: 698 - protein, 386 aa, Equid alphaherpesvirus 1
TQKLSLGLYNQWWRVCRSVPPPWYVFFNKRSMSTFKLMMDGRLVFAMAIAILSVVLSCGTCEKAKRAVRGRQDRPKEFPPPRYNYTILTRYNATALASPFINDQVKNVDLRIVTATRPCEMIALIAKTNIDSILKELAAAQKTYSARLTWFKIMPTCATPIHDVSYMKCNPKLSFAMCDERSDILWQASLITMAAETDDELGLVLAAPAHSASGLYRRVIEIDGRRIYTDFSVTIPSERCPIAFEQNFGNPDRCKTPEQYSRGEVFTRRFLGEFNFPQGEHMTWLKFWFVYDGGNLPVQFYEAQAFARPVPPDNHPGFDSVESEITQNKTDPKPGQADPKPNQPFKWPSIKHLAPRLDEVDEVIEPVTKPPKTSKSNSTFVGISVG

SEQ ID NO: 699 - protein, 433 aa, Equid alphaherpesvirus 1
TQKLSLGLYNQWWRVCRSVPPPWYVFFNKRSMSTFKLMMDGRLVFAMAIAILSVVLSCGTCEKAKRAVRGRQDRPKEFPPPRYNYTILTRYNATALASPFINDQVKNVDLRIVTATRPCEMIALIAKTNIDSILKELAAAQKTYSARLTWFKIMPTCATPIHDVSYMKCNPKLSFAMCDERSDILWQASLITMAAETDDELGLVLAAPAHSASGLYRRVIEIDGRRIYTDFSVTIPSERCPIAFEQNFGNPDRCKTPEQYSRGEVFTRRFLGEFNFPQGEHMTWLKFWFVYDGGNLPVQFYEAQAFARPVPPDNHPGFDSVESEITQNKTDPKPGQADPKPNQPFKWPSIKHLAPRLDEVDEVIEPVTKPPKTSKSNSTFVGISVGLGIAGLVLVGVILYVCLRRKKELKKSAQNGLTRLRSTFKDVKYTQLP

SEQ ID NO: 700 - protein, 19 aa, Equid alphaherpesvirus 1
MPAVLLVLYVNPPPSVCIL

SEQ ID NO: 701 - protein, 16 aa, Severe acute respiratory syndrome-related coronavirus
NYNYKYRYLRGKLRPF

SEQ ID NO: 702 - protein, 15 aa, Severe acute respiratory syndrome-related coronavirus
PLQASLPFGWLVIGV

SEQ ID NO: 703 - protein, 10 aa, Severe acute respiratory syndrome-related coronavirus
TLACFVLAAV

SEQ ID NO: 704 - protein, 10 aa, Severe acute respiratory syndrome-related coronavirus
GYQPYRVVVL

SEQ ID NO: 705 - protein, 10 aa, Severe acute respiratory syndrome-related coronavirus
QPYRVVVLSF

SEQ ID NO: 706 - protein, 9 aa, Severe acute respiratory syndrome-related coronavirus
PYRVVVLSF

SEQ ID NO: 707 - protein, 15 aa, Severe acute respiratory syndrome-related coronavirus
KPSFYVYSRVKNLNS

SEQ ID NO: 708 - protein, 15 aa, Severe acute respiratory syndrome-related coronavirus
NIVNVSLVKPSFYVY

SEQ ID NO: 709 - protein, 9 aa, Severe acute respiratory syndrome-related coronavirus
VSDGGPNLY

SEQ ID NO: 710 - protein, 15 aa, Severe acute respiratory syndrome-related coronavirus
VIGAVILRGHLRIAG

SEQ ID NO: 711 - protein, 15 aa, Severe acute respiratory syndrome-related coronavirus
FNATRFASVYAWNRK

SEQ ID NO: 712 - protein, 15 aa, Severe acute respiratory syndrome-related coronavirus
ADSFVIRGDEVRQIA

SEQ ID NO: 713 - protein, 15 aa, Severe acute respiratory syndrome-related coronavirus
ISNCVADYSVLYNSA

SEQ ID NO: 714 - protein, 15 aa, Severe acute respiratory syndrome-related coronavirus
IWLGFIAGLIAIVMV

SEQ ID NO: 715 - protein, 15 aa, Severe acute respiratory syndrome-related coronavirus
FLHVTYVPAQEKNFT

SEQ ID NO: 716 - protein, 98 aa, synthetic construct
  REGION 1..98: note = SYNTHETIC CONSTRUCT DERIVED FROM HUMAN THOUGHT
MHGDTPTLHEYMLDLQPETTDLYCYEQLDDSSEEEDEIDGPAGQAEPDRAHYNIVTFCCKCDSTLRLCVQSTHVDIRTLEDLLMGTLGIVCPICSQKP

SEQ ID NO: 717 - protein, 9 aa, Homo sapiens
IYNEYIYDL

SEQ ID NO: 718 - protein, 9 aa, Homo sapiens
EYYELFVNI

SEQ ID NO: 719 - protein, 19 aa, synthetic construct
  REGION 1..19: note = SYNTHETIC CONSTRUCT DERIVED FROM HUMAN THOUGHT
  VARIANT 3: note = MISC_FEATURE - Xaa3 IS ANY AMINO ACID
  VARIANT 4: note = MISC_FEATURE - Xaa4 IS ANY AMINO ACID
  VARIANT 6: note = MISC_FEATURE - Xaa6 IS ANY AMINO ACID
  VARIANT 8: note = MISC_FEATURE - Xaa8 IS ANY AMINO ACID
  VARIANT 9: note = MISC_FEATURE - Xaa9 IS ANY AMINO ACID
  VARIANT 10: note = MISC_FEATURE - Xaa10 IS ANY AMINO ACID
  VARIANT 11: note = MISC_FEATURE - Xaa11 IS ANY AMINO ACID
  VARIANT 14: note = MISC_FEATURE - Xaa14 IS ANY AMINO ACID
  VARIANT 15: note = MISC_FEATURE - Xaa15 IS ANY AMINO ACID
MAXXNXFXXXXLPXXDRLT

SEQ ID NO: 720 - protein, 22 aa, synthetic construct
  REGION 1..22: note = SYNTHETIC CONSTRUCT DERIVED FROM HUMAN THOUGHT
  VARIANT 2: note = MISC_FEATURE - Xaa2 IS ANY AMINO ACID
  VARIANT 3: note = MISC_FEATURE - Xaa3 IS Asn OR Gly
  VARIANT 5: note = MISC_FEATURE - Xaa5 IS ANY AMINO ACID OR IS ABSENT
  VARIANT 6: note = MISC_FEATURE - Xaa6 IS ANY AMINO ACID OR IS ABSENT
  VARIANT 9: note = MISC_FEATURE - Xaa9 IS Ile or Leu
  VARIANT 10: note = MISC_FEATURE - Xaa10 IS Gln OR Glu
  VARIANT 11: note = MISC_FEATURE - Xaa11 IS ANY AMINO ACID
  VARIANT 12: note = MISC_FEATURE - Xaa12 IS ANY AMINO ACID
  VARIANT 13: note = MISC_FEATURE - Xaa13 IS Ala OR Thr
  VARIANT 14: note = MISC_FEATURE - Xaa14 IS ANY AMINO ACID
  VARIANT 16: note = MISC_FEATURE - Xaa16 IS ANY AMINO ACID
  VARIANT 17: note = MISC_FEATURE - Xaa17 IS ANY AMINO ACID
  VARIANT 18: note = MISC_FEATURE - Xaa18 IS ANY AMINO ACID
PXXWXXPSXXXXXXPXXXPATP

SEQ ID NO: 721 - protein, 29 aa, synthetic construct
  REGION 1..29: note = SYNTHETIC CONSTRUCT DERIVED FROM HUMAN THOUGHT
  VARIANT 2: note = MISC_FEATURE - Xaa2 IS ANY AMINO ACID
  VARIANT 3: note = MISC_FEATURE - Xaa3 IS ANY AMINO ACID
  VARIANT 4: note = MISC_FEATURE - Xaa4 IS ANY AMINO ACID
  VARIANT 5: note = misc_feature - Xaa5 can be any naturally occurring amino acid
  VARIANT 8: note = MISC_FEATURE - Xaa8 IS ANY AMINO ACID OR IS ABSENT
  VARIANT 9: note = MISC_FEATURE - Xaa9 IS ANY AMINO ACID
  VARIANT 10: note = MISC_FEATURE - Xaa10 IS ANY AMINO ACID
  VARIANT 11: note = MISC_FEATURE - Xaa11 IS Ile OR Val
  VARIANT 13: note = MISC_FEATURE - Xaa13 IS ANY AMINO ACID
  VARIANT 14: note = MISC_FEATURE - Xaa14 IS Asn OR Gly
  VARIANT 16: note = MISC_FEATURE - Xaa16 IS ANY AMINO ACID
  VARIANT 17: note = MISC_FEATURE - Xaa17 IS ANY AMINO ACID
  VARIANT 19: note = MISC_FEATURE - Xaa19 IS Ser OR Ala
  VARIANT 20: note = MISC_FEATURE - Xaa20 IS ANY AMINO ACID
  REGION 20..28: note = MISC_FEATURE - Xaa20-Xaa28 COMPRISES 1-4 Pro RESIDUES
  VARIANT 21: note = MISC_FEATURE - Xaa21 IS ANY AMINO ACID
  VARIANT 22: note = MISC_FEATURE - Xaa22 IS ANY AMINO ACID
  VARIANT 23: note = MISC_FEATURE - Xaa23 IS ANY AMINO ACID
  VARIANT 24: note = MISC_FEATURE - Xaa24 IS ANY AMINO ACID
  VARIANT 25: note = MISC_FEATURE - Xaa25 IS ANY AMINO ACID
  VARIANT 26: note = MISC_FEATURE - Xaa26 IS ANY AMINO ACID
  VARIANT 27: note = MISC_FEATURE - Xaa27 IS ANY AMINO ACID
  VARIANT 28: note = MISC_FEATURE - Xaa28 IS ANY AMINO ACID
PXXXXAPXXXXPXXWXXPXXXXXXXXXXP

SEQ ID NO: 722 - protein, 13 aa, synthetic construct
  REGION 1..13: note = SYNTHETIC CONSTRUCT DERIVED FROM HUMAN THOUGHT
QIPPNWHIPSIQD

SEQ ID NO: 723 - protein, 132 aa, synthetic construct
  REGION 1..132: note = SYNTHETIC CONSTRUCT DERIVED FROM HUMAN THOUGHT
  VARIANT 9: note = MISC_FEATURE - Xaa9 IS ANY AMINO ACID
  VARIANT 13: note = MISC_FEATURE - Xaa13 IS Gln OR Arg
  VARIANT 14: note = MISC FEATURE - Xaa14 IS Asp OR Glu
  VARIANT 17: note = MISC_FEATURE - Xaa17 IS Thr OR Ala
  VARIANT 22: note = MISC_FEATURE - Xaa22 IS ANY AMINO ACID
  VARIANT 30: note = MISC_FEATURE - Xaa30 IS Leu OR Ile
  VARIANT 36: note = MISC_FEATURE - Xaa36 IS Ile OR Val
  VARIANT 39: note = MISC_FEATURE - Xaa39 IS Val OR Ala
  VARIANT 48: note = MISC_FEATURE - Xaa48 IS Asn OR Lys
  VARIANT 49: note = MISC_FEATURE - Xaa49 IS Ile OR Leu
  VARIANT 52: note = MISC_FEATURE - Xaa52 IS Thr OR Ser
  VARIANT 68: note = MISC_FEATURE - Xaa68 IS ANY AMINO ACID
  VARIANT 69: note = MISC_FEATURE - Xaa69 IS ANY AMINO ACID
  VARIANT 70: note = MISC_FEATURE - Xaa70 IS ANY AMINO ACID
  VARIANT 71: note = MISC_FEATURE - Xaa71 IS Ser OR Thr
  SITE 73: note = MISC_FEATURE - Xaa73 IS Lys OR Arg
  VARIANT 74: note = MISC_FEATURE - Xaa74 IS ANY AMINO ACID
  VARIANT 75: note = MISC_FEATURE - Xaa75 IS Val OR Ile
  VARIANT 78: note = MISC_FEATURE - Xaa78 IS Ser OR Asn
  VARIANT 80: note = MISC_FEATURE - Xaa80 IS Lys OR Gln
  VARIANT 83
  VARIANT 86: note = MISC_FEATURE - Xaa86 IS Phe OR Tyr
  VARIANT 87: note = MISC_FEATURE - Xaa87 IS ANY AMINO ACID
  VARIANT 90: note = MISC_FEATURE - Xaa90 IS Asn OR Gly
  VARIANT 93: note = MISC_FEATURE - Xaa93 IS Met OR Leu
  VARIANT 98: note = MISC_FEATURE - Xaa98 IS ANY AMINO ACID
  VARIANT 99: note = MISC_FEATURE - Xaa99 IS Lys OR Asn
  VARIANT 102: note = MISC_FEATURE - Xaa102 IS ANY AMINO ACID
  VARIANT 104: note = MISC_FEATURE - Xaa104 IS Thr OR Asn
  VARIANT 105: note = MISC_FEATURE - Xaa105 IS Ser OR Asn
  VARIANT 106: note = MISC_FEATURE - Xaa106 IS ANY AMINO ACID
  VARIANT 107: note = MISC_FEATURE - Xaa107 IS ANY AMINO ACID
  VARIANT 108: note = MISC_FEATURE - Xaa108 IS ANY AMINO ACID
  VARIANT 110: note = MISC_FEATURE - Xaa110 IS ANY AMINO ACID
  VARIANT 113: note = MISC_FEATURE - Xaa113 IS Leu OR Met
  VARIANT 114: note = MISC_FEATURE - Xaa114 IS Lys OR Glu
  VARIANT 118: note = MISC_FEATURE - Xaa118 IS Glu OR Asn
  VARIANT 119: note = MISC_FEATURE - Xaa119 IS Thr OR Val
  VARIANT 120: note = MISC_FEATURE - Xaa120 IS Phe OR Tyr
  VARIANT 121: note = MISC_FEATURE - Xaa121 IS ANY AMINO ACID
  VARIANT 123: note = MISC_FEATURE - Xaa123 IS Ser OR Thr
  SITE 124: note = MISC_FEATURE - Xaa124 IS Asn OR Asp
  VARIANT 125: note = MISC_FEATURE - Xaa125 IS ANY AMINO ACID
  VARIANT 126: note = MISC_FEATURE - Xaa126 IS Asp OR Glu
MDLPYYHGXLTKXXCEXLLLKXGVDGNFLXRDSESXPGXLCLCVSFKXXVYXYRIFREKHGYYRIQTXXXXPXXXFPXLXELXSKXXKPXQGXVVHLXXPIXRXXXXXRXRGXXLELXXXXNXXXXYVDVLP

SEQ ID NO: 724 - protein, 296 aa, Porcine circovirus 3
VRRESPKHRWCFTINDWTPTEWESIVECGGSIARYLIIGKEVGKSGTPHLQGYVNFKNKRRLSSVKRLPGFGRAHLEPARGSHKEASEYCKKEGDYLEIGEDSSSGTRSDLQAAARILTETAGNLTEVAEKMPAVFIRYGRGLRDFCGVMGLGKPRDFKTEVYVFIGPPGCGKTREACADAAARELQLYFKPRGPWWDGYNGEGAVILDDFYGWVPFDELLRIGDRYPLRVPVKGGFVNFVAKVLYITSNVVPEEWYSSENIRGKLEALFRRFTKVVCWGEGGVKKDMETVYPINY

SEQ ID NO: 725 - protein, 296 aa, Porcine circovirus 3
VRRESPKHRWCFTINNWTPTEWESIVECGGSIARYLIIGKEVGKSGTPHLQGYVNFKNKRRLSSVKRLPGFGRAHLEPARGSHKEASEYCKKEGDYLEIGEDSSSGTRSDLQAAARILTETAGDLTEVAEKMPAVFIRYGRGLRDFCGVMGLGKPRDFKTEVYVFIGPPGCGKTREACADAAARELQLYFKPRGPWWDGYNGEGAVILDDFYGWVPFDELLRIGDRYPLRVPVKGGFVNFVAKVLYITSNVVPEEWYSSENIRGKLEALFRRFTKVVCWGEGGVKKDMETVYPINY

SEQ ID NO: 726 - protein, 296 aa, Porcine circovirus 3
VRRESPKHRWCFTINDWTPTEWESIVECGGSIARYLIIGKEVGKSGTPHLQGYVNFKNKRRLSSVKRLPGFGRAHLEPARGSHKEASEYCKKEGDYLEIGEDSSSGTRSDLQAAARILTETAGDLTEVAEKMPAVFIRYGRGLRDFCGVMGLGKPRDFKTEVYVFIGPPGCGKTREACADAAARELQLYFKPRGPWWDGYNGEGAVILDDFYGWVPFDELLRIGDRYPLRVPVKGGFVNFVAKVLYITSNVVPEEWYSSENIRGKLEALFRRFTKVVCWGEGGVKKDMETVYPINY

SEQ ID NO: 727 - protein, 296 aa, Porcine circovirus 3
VRRESPKHRWCFTINNWTPTEWESIVECGGSIARYLIIGKEVGKSGTPHLQGYVNFKNKRRLSSVKRLPGFGRAHLEPARGSHKEASEYCKKEGDYLEIGEDSSSGTRSDLQAAARILTETAGNLTEVAEKMPAVFIRYGRGLRDFCGVMGLGKPRDFKTEVYVFIGPPGCGKTREACADAAARELQLYFKPRGPWWDGYNGEGAVILDDFYGWVPFDELLRIGDRYPLRVPVKGGFVNFVAKVLYITSNVVPEEWYSSENIRGKLEALFRRFTKVVCWGEGGVKKDMETVYPINY

SEQ ID NO: 728 - protein, 214 aa, Porcine circovirus 3
MRHRAIFRRRPRPRRRRRHRRRYVRRKLFIRRPTAGTYYTKKYSTMNVISVGTPQNNKPWHANHFITRLNEWETAISFEYYKILKMKVTLSPVISPAQQTKTMFGHTAIDLDGAWTTNTWLQDDPYAESSTRKVMTSKKKHSRYFTPKPILAGTTSAHPGQSLFFFSRPTPWLNTYDPTVQWGALLWSIYVPEKTGMTDFYGTKEVWIRYKSVL

SEQ ID NO: 729 - protein, 10 aa, Human immunodeficiency virus 1
RKKRRQRRRR

SEQ ID NO: 730 - protein, 5 aa, synthetic construct
  REGION 1..5: note = SYNTHETIC CONSTRUCT DERIVED FROM HUMAN THOUGHT
GPGPG

SEQ ID NO: 731 - protein, 5 aa, synthetic construct
  REGION 1..5: note = SYNTHETIC CONSTRUCT DERIVED FROM HUMAN THOUGHT
YVPGP

SEQ ID NO: 732 - protein, 5 aa, Homo sapiens
YVPGP

SEQ ID NO: 733 - protein, 7 aa, Influenza A
KRRSNKS

SEQ ID NO: 734 - protein, 15 aa, African swine fever virus
SFLNLTKLYHHHSHY

SEQ ID NO: 735 - protein, 15 aa, African swine fever virus
KYNLNRKKSHYTDLL

SEQ ID NO: 736 - protein, 15 aa, African swine fever virus
NRKKSHYTDLLFICS

SEQ ID NO: 737 - protein, 15 aa, African swine fever virus
SPPPKPCPPPKPCPP

SEQ ID NO: 738 - protein, 15 aa, African swine fever virus
KPCPPPKPCPPPKPC

SEQ ID NO: 739 - protein, 18 aa, African swine fever virus
PEERCTYKFNSYTKKMEL

SEQ ID NO: 740 - protein, 19 aa, African swine fever virus
DQEEKKALQNKETKNLGIP

SEQ ID NO: 741 - protein, 9 aa, Homo sapiens
KQMNDARHG

SEQ ID NO: 742 - protein, 9 aa, Homo sapiens
LSKITEQEK

SEQ ID NO: 743 - protein, 9 aa, Homo sapiens
STRDPLSKI

SEQ ID NO: 744 - protein, 138 aa, synthetic construct
  REGION 1..138: note = Synthetic construct derived from human thought
  VARIANT 1: note = MISC_FEATURE - Xaa1 IS Val, Thr, OR Asn
  VARIANT 2: note = MISC_FEATURE - Xaa2 IS ANY AMINO ACID
  VARIANT 3: note = MISC_FEATURE - Xaa3 IS Tyr OR His
  VARIANT 4: note = MISC_FEATURE - Xaa4 IS Ala, Thr, Val, OR Leu
  VARIANT 5: note = MISC_FEATURE - Xaa5 IS Val, Thr, Arg, OR Ile
  VARIANT 6: note = MISC_FEATURE - Xaa6 IS ANY AMINO ACID
  VARIANT 7: note = MISC_FEATURE - Xaa7 IS ANY AMINO ACID
  VARIANT 8: note = MISC_FEATURE - Xaa8 IS ANY AMINO ACID OR IS ABSENT
  VARIANT 9: note = MISC_FEATURE - Xaa9 IS Arg, Ala, Asp, OR Glu
  VARIANT 10: note = MISC_FEATURE - Xaa10 IS ANY AMINO ACID
  VARIANT 12: note = MISC_FEATURE - Xaa12 IS Arg, Asp, Gly, OR Ser
  VARIANT 13: note = MISC_FEATURE - Xaa13 IS Ser, Met, Val, OR Phe
  VARIANT 14: note = MISC_FEATURE - Xaa14 IS Val, Leu, OR Ala
  VARIANT 15: note = MISC_FEATURE - Xaa15 IS ANY AMINO ACID
  VARIANT 17: note = MISC_FEATURE - Xaa17 IS Asn, Ile, OR Trp
  VARIANT 18: note = MISC_FEATURE - Xaa18 IS Ala, Ser, OR Pro
  VARIANT 19: note = MISC_FEATURE - Xaa19 IS ANY AMINO ACID
  VARIANT 20: note = MISC_FEATURE - Xaa20 IS Ser, Pro, Thr, OR Gly
  VARIANT 21: note = MISC_FEATURE - Xaa21 IS Glu, Gln, Asp, OR Asn
  VARIANT 22: note = MISC_FEATURE - Xaa22 IS Ala, Val, Pro, OR Ile
  VARIANT 23: note = MISC_FEATURE - Xaa23 IS ANY AMINO ACID
  VARIANT 24: note = MISC_FEATURE - Xaa24 IS ANY AMINO ACID
  VARIANT 25: note = MISC_FEATURE - Xaa25 IS Ile, Thr, Leu, Val, OR Met
  VARIANT 26: note = MISC_FEATURE - Xaa26 IS Val, Leu, OR Ile
  VARIANT 27: note = MISC_FEATURE - Xaa27 IS ANY AMINO ACID
  VARIANT 28: note = MISC_FEATURE - Xaa28 IS ANY AMINO ACID
  VARIANT 29: note = MISC_FEATURE - Xaa29 IS Ala, Leu, Gln, OR Ile
  VARIANT 30: note = MISC_FEATURE - Xaa30 IS ANY AMINO ACID OR IS ABSENT
  VARIANT 31: note = MISC_FEATURE - Xaa31 IS ANY AMINO ACID OR IS ABSENT
  VARIANT 32: note = MISC_FEATURE - Xaa32 IS ANY AMINO ACID OR IS ABSENT
  VARIANT 33: note = MISC_FEATURE - Xaa33 IS ANY AMINO ACID OR IS ABSENT
  VARIANT 34: note = MISC_FEATURE - Xaa34 IS ANY AMINO ACID OR IS ABSENT
  VARIANT 35: note = MISC_FEATURE - Xaa35 IS ANY AMINO ACID OR IS ABSENT
  VARIANT 36: note = MISC_FEATURE - Xaa36 IS Pro, Val, Ser, Leu, OR Gln
  VARIANT 37: note = MISC_FEATURE - Xaa37 IS ANY AMINO ACID OR IS ABSENT
  VARIANT 38: note = MISC_FEATURE - Xaa38 IS ANY AMINO ACID OR IS ABSENT
  VARIANT 39: note = MISC_FEATURE - Xaa39 IS ANY AMINO ACID OR IS ABSENT
  VARIANT 40: note = MISC_FEATURE - Xaa40 IS ANY AMINO ACID OR IS ABSENT
  VARIANT 41: note = MISC_FEATURE - Xaa41 IS ANY AMINO ACID OR IS ABSENT
  VARIANT 42: note = MISC_FEATURE - Xaa42 IS ANY AMINO ACID OR IS ABSENT
  VARIANT 43: note = MISC_FEATURE - Xaa43 IS ANY AMINO ACID OR IS ABSENT
  VARIANT 44: note = MISC_FEATURE - Xaa44 IS Tyr OR Phe
  VARIANT 45: note = MISC_FEATURE - Xaa45 IS Asn, Arg, Asp, OR Glu
  VARIANT 46: note = MISC_FEATURE - Xaa46 IS Leu OR Ala
  VARIANT 47: note = MISC_FEATURE - Xaa47 IS Thr, His, Leu, OR Ser
  VARIANT 48: note = MISC_FEATURE - Xaa48 IS Ile, Val, OR Leu
  VARIANT 49: note = MISC_FEATURE - Xaa49 IS Ala, Ile, Ser OR Val
  VARIANT 51: note = MISC_FEATURE - Xaa51 IS Phe OR Tyr
  VARIANT 52: note = MISC_FEATURE - Xaa52 IS Arg, Lys, OR Val
  VARIANT 53: note = misc_feature - Xaa53 can be any naturally occurring amino acid
  VARIANT 54: note = MISC_FEATURE - Xaa54 IS ANY AMINO ACID
  VARIANT 55: note = MISC_FEATURE - Xaa55 IS ANY AMINO ACID
  VARIANT 56: note = MISC_FEATURE - Xaa56 IS Asn, Gly, Asp, Ala, OR Met
  VARIANT 57: note = MISC_FEATURE - Xaa57 IS ANY AMINO ACID OR IS ABSENT
  VARIANT 58: note = MISC_FEATURE - Xaa58 IS ANY AMINO ACID OR IS ABSENT
  VARIANT 59: note = MISC_FEATURE - Xaa59 IS ANY AMINO ACID OR IS ABSENT
  VARIANT 60: note = MISC_FEATURE - Xaa60 IS ANY AMINO ACID OR IS ABSENT
  VARIANT 61: note = MISC_FEATURE - Xaa61 IS ANY AMINO ACID OR IS ABSENT
  VARIANT 62: note = MISC_FEATURE - Xaa62 IS ANY AMINO ACID OR IS ABSENT
  VARIANT 64: note = MISC_FEATURE - Xaa64 IS Ala, Gly, Glu, OR Thr OR IS ABSENT
  VARIANT 65: note = MISC_FEATURE - Xaa7 IS ANY AMINO ACID
  VARIANT 66: note = MISC_FEATURE - Xaa66 IS Pro OR Leu
  VARIANT 67: note = MISC_FEATURE - Xaa67 IS Ile, Leu, OR Met
  VARIANT 68: note = MISC_FEATURE - Xaa68 IS Thr, Tyr, Leu, OR Phe
  VARIANT 69: note = MISC_FEATURE - Xaa69 IS ANY AMINO ACID
  VARIANT 70: note = MISC_FEATURE - Xaa70 IS Met, Ile, Arg, OR Lys
  VARIANT 71: note = MISC_FEATURE - Xaa71 IS Glu, Asp, OR Gln
  VARIANT 72: note = MISC_FEATURE - Xaa72 IS Tyr, Phe, OR Met
  VARIANT 73: note = MISC_FEATURE - Xaa73 IS ANY AMINO ACID
  VARIANT 74: note = MISC_FEATURE - Xaa74 IS Glu, Asp, Asn, OR Leu
  VARIANT 76: note = MISC_FEATURE - Xaa76 IS ANY AMINO ACID
  VARIANT 77: note = MISC_FEATURE - Xaa77 IS Tyr, Pro, Thr, OR Asp
  VARIANT 78: note = MISC_FEATURE - Xaa78 IS Asn, Arg, Asp, Lys, Gln, OR Val
  VARIANT 79: note = MISC_FEATURE - Xaa79 IS Lys, Gln, Glu, OR Arg
  VARIANT 80: note = MISC_FEATURE - Xaa80 IS ANY AMINO ACID
  VARIANT 81: note = MISC_FEATURE - Xaa81 IS Leu OR Phe
  VARIANT 82: note = MISC_FEATURE - Xaa82 IS Gly OR Ser
  VARIANT 83: note = MISC_FEATURE - Xaa83 IS ANY AMINO ACID
  VARIANT 85: note = MISC_FEATURE - Xaa85 IS ANY AMINO ACID
  VARIANT 86: note = MISC_FEATURE - Xaa86 IS ANY AMINO ACID
  VARIANT 87: note = MISC_FEATURE - Xaa87 IS Arg, Lys, Ser, OR Gln
  VARIANT 88: note = MISC_FEATURE - Xaa88 IS Thr, Asn, OR Ser
  VARIANT 89: note = MISC_FEATURE - Xaa89 IS ANY AMINO ACID
  VARIANT 90: note = MISC_FEATURE - Xaa90 IS Pro, Gly, Ser, Asp, OR Gln
  VARIANT 91: note = MISC_FEATURE - Xaa91 IS Arg, Phe, Met, Trp, OR Tyr
  VARIANT 93: note = MISC_FEATURE - Xaa93 IS ANY AMINO ACID
  VARIANT 94: note = MISC_FEATURE - Xaa94 IS ANY AMINO ACID OR IS ABSENT
  VARIANT 95: note = MISC_FEATURE - Xaa95 IS ANY AMINO ACID
  VARIANT 96: note = MISC_FEATURE - Xaa96 IS Tyr, Leu, OR Ser
  VARIANT 97: note = MISC_FEATURE - Xaa97 IS Asp, Ala, Val, OR Thr
  VARIANT 98: note = MISC_FEATURE - Xaa98 IS ANY RESIDUE
  VARIANT 99: note = MISC_FEATURE - Xaa99 IS Phe, Tyr, Thr, OR Ser
  VARIANT 100: note = MISC_FEATURE - Xaa100 IS Ser, Ala, Met, OR Thr
  VARIANT 101: note = MISC_FEATURE - Xaa101 IS Ala, Tyr, Phe, OR Leu
  VARIANT 102: note = MISC_FEATURE - Xaa102 IS ANY RESIDUE
  VARIANT 103: note = MISC_FEATURE - Xaa103 IS Ser, Thr, OR Gly
  VARIANT 104: note = MISC_FEATURE - Xaa104 IS Glu, Asp, OR Arg
  VARIANT 105: note = MISC_FEATURE - Xaa105 IS Asp OR Asn
  VARIANT 106: note = MISC_FEATURE - Xaa106 IS Asn, Glu, OR Gly
  VARIANT 107: note = MISC_FEATURE - Xaa107 IS Leu OR Ala
  VARIANT 109: note = MISC_FEATURE - Xaa109 IS Phe OR Leu
  VARIANT 110: note = MISC_FEATURE - Xaa110 IS Leu, Ile, Val, OR Thr
  VARIANT 111: note = MISC_FEATURE - Xaa111 IS Met, Ile, Phe, OR Leu
  VARIANT 112: note = MISC_FEATURE - Xaa112 IS His, Ala, Val, Met, OR Phe
  VARIANT 113: note = MISC_FEATURE - Xaa113 IS Ala OR Ser
  VARIANT 115: note = MISC_FEATURE - Xaa115 IS Phe, Arg, Gln, Pro, OR Ala
  VARIANT 116: note = MISC_FEATURE - Xaa116 IS Glu, Leu, Phe, OR Ala
  VARIANT 117: note = MISC_FEATURE - Xaa117 IS Thr, Val, Asn, OR Leu
  VARIANT 118: note = MISC_FEATURE - Xaa118 IS ANY AMINO ACID
  VARIANT 120: note = MISC_FEATURE - Xaa120 IS Thr, Gln, OR Leu
  VARIANT 122: note = MISC_FEATURE - Xaa122 IS Leu, Arg, OR Thr
  VARIANT 123: note = MISC_FEATURE - Xaa123 IS Arg OR Leu
  VARIANT 124: note = MISC_FEATURE - Xaa124 IS Leu, Ala, Val, OR Thr
  VARIANT 125: note = MISC_FEATURE - Xaa125 IS Val, Leu, OR Ile
  VARIANT 126: note = MISC_FEATURE - Xaa126 IS ANY AMINO ACID
  VARIANT 127: note = MISC_FEATURE - Xaa127 IS Ile OR Val
  VARIANT 128: note = MISC_FEATURE - Xaa128 IS Asn, Asp, Glu, OR Gly
  VARIANT 129: note = MISC_FEATURE - Xaa129 IS Asp, Gly, Asn, OR Arg
  VARIANT 130: note = MISC_FEATURE - Xaa130 IS Trp, Thr, Val, Glu, Asp, Ser, OR Phe
  VARIANT 131: note = MISC_FEATURE - Xaa131 IS ANY AMINO ACID
  VARIANT 132: note = MISC_FEATURE - Xaa132 IS ANY AMINO ACID
  VARIANT 133: note = MISC_FEATURE - Xaa133 IS ANY AMINO ACID
  VARIANT 134: note = MISC_FEATURE - Xaa134 IS Thr, Ser, OR Ala
  VARIANT 135: note = MISC_FEATURE - Xaa135 IS Gln, Asp, OR Leu
  VARIANT 136: note = MISC_FEATURE - Xaa136 IS Phe, Ile, OR Val
  VARIANT 137: note = MISC_FEATURE - Xaa137 IS Ile, Met, Leu, OR Thr
  VARIANT 138: note = MISC_FEATURE - Xaa138 IS Leu, Val, OR Phe
XXXXXXXXXXCXXXXLXXXXXXXXXXXXXXXXXXXXXXXXXXXXXXXXXWXXXXXXXXXXXXCXXXXXXXXXXXCXXXXXXXXCXXXXXXXWXXXXXXXXXXXXXXXGXXXXXPXXXXGXYXXXXXXXXXXXXXXXXX

SEQ ID NO: 745 - protein, 138 aa, synthetic construct; identical to SEQ ID NO: 744
  REGION 1..138: note = Synthetic construct derived from human thought
  VARIANT 1: note = MISC_FEATURE - Xaa1 IS Val, Thr, OR Asn
  VARIANT 2: note = MISC_FEATURE - Xaa2 IS Tyr, Arg, OR Lys
  VARIANT 3: note = MISC_FEATURE - Xaa3 IS Tyr OR His
  VARIANT 4: note = MISC_FEATURE - Xaa4 IS Ala, Thr, Val, OR Leu
  VARIANT 5: note = MISC_FEATURE - Xaa5 IS Val, Thr, Arg, OR Ile
  VARIANT 6: note = MISC_FEATURE - Xaa6 IS Leu, Ser, Pro, Tyr, OR Arg
  VARIANT 7: note = MISC_FEATURE - Xaa7 IS Glu, Ala, Leu, Thr, Asp, Met, OR Val
  VARIANT 8: note = MISC_FEATURE - Xaa8 IS ANY AMINO ACID OR IS ABSENT
  VARIANT 9: note = MISC_FEATURE - Xaa9 IS Arg, Ala, Asp, OR Glu
  VARIANT 10: note = MISC_FEATURE - Xaa10 IS Ala, Pro, Asn, Ser, OR Gly
  VARIANT 12: note = MISC_FEATURE - Xaa12 IS Arg, Asp, Gly, OR Ser
  VARIANT 13: note = MISC_FEATURE - Xaa13 IS Ser, Met, Val, OR Phe
  VARIANT 14: note = MISC_FEATURE - Xaa14 IS Val, Leu, OR Ala
  VARIANT 15: note = MISC_FEATURE - Xaa15 IS Leu, Ala, Val, OR Glu
  VARIANT 17: note = MISC_FEATURE - Xaa17 IS Asn, Ile, OR Trp
  VARIANT 18: note = MISC_FEATURE - Xaa18 IS Ala, Ser, OR Pro
  VARIANT 19: note = MISC_FEATURE - Xaa19 IS Pro, Asp, Phe, Ile, Glu, OR Asn
  VARIANT 20: note = MISC_FEATURE - Xaa20 IS Ser, Pro, Thr, OR Gly
  VARIANT 21: note = MISC_FEATURE - Xaa21 IS Glu, Gln, Asp, OR Asn
  VARIANT 22: note = MISC_FEATURE - Xaa22 IS Ala, Val, Pro, OR Ile
  VARIANT 23: note = MISC_FEATURE - Xaa23 IS Pro, Gly, Asp, Val, Lys, OR Glu
  VARIANT 24: note = MISC_FEATURE - Xaa24 IS Gln, Arg, Tyr, Ala, Ser, OR Asp
  VARIANT 25: note = MISC_FEATURE - Xaa25 IS Ile, Thr, Leu, Val, OR Met
  VARIANT 26: note = MISC_FEATURE - Xaa26 IS Val, Leu, OR Ile
  VARIANT 27: note = MISC_FEATURE - Xaa27 IS Arg, Trp, Asn, Leu, OR Ser
  VARIANT 28: note = MISC_FEATURE - Xaa28 IS Gly, Glu, Ser, Thr, OR Ala
  VARIANT 29: note = MISC_FEATURE - Xaa29 IS Ala, Leu, Gln, OR Ile
  VARIANT 30: note = MISC_FEATURE - Xaa30 IS ANY AMINO ACID OR IS ABSENT
  VARIANT 31: note = MISC_FEATURE - Xaa31 IS ANY AMINO ACID OR IS ABSENT
  VARIANT 32: note = MISC_FEATURE - Xaa32 IS ANY AMINO ACID OR IS ABSENT
  VARIANT 33: note = MISC_FEATURE - Xaa33 IS ANY AMINO ACID OR IS ABSENT
  VARIANT 34: note = MISC_FEATURE - Xaa34 IS ANY AMINO ACID OR IS ABSENT
  VARIANT 35: note = MISC_FEATURE - Xaa35 IS ANY AMINO ACID OR IS ABSENT
  VARIANT 36: note = MISC_FEATURE - Xaa36 IS Pro, Val, Ser, Leu, OR Gln
  VARIANT 37: note = MISC_FEATURE - Xaa37 IS ANY AMINO ACID OR IS ABSENT
  VARIANT 38: note = MISC_FEATURE - Xaa38 IS ANY AMINO ACID OR IS ABSENT
  VARIANT 39: note = MISC_FEATURE - Xaa39 IS ANY AMINO ACID OR IS ABSENT
  VARIANT 40: note = MISC_FEATURE - Xaa40 IS ANY AMINO ACID OR IS ABSENT
  VARIANT 41: note = MISC_FEATURE - Xaa41 IS ANY AMINO ACID OR IS ABSENT
  VARIANT 42: note = MISC_FEATURE - Xaa42 IS ANY AMINO ACID OR IS ABSENT
  VARIANT 43: note = MISC_FEATURE - Xaa43 IS ANY AMINO ACID OR IS ABSENT
  VARIANT 44: note = MISC_FEATURE - Xaa44 IS Tyr OR Phe
  VARIANT 45: note = MISC_FEATURE - Xaa45 IS Asn, Arg, Asp, OR Glu
  VARIANT 46: note = MISC_FEATURE - Xaa46 IS Leu OR Ala
  VARIANT 47: note = MISC_FEATURE - Xaa47 IS Thr, His, Leu, OR Ser
  VARIANT 48: note = MISC_FEATURE - Xaa48 IS Ile, Val, OR Leu
  VARIANT 49: note = MISC_FEATURE - Xaa49 IS Ala, Ile, Ser, OR Val
  VARIANT 51: note = MISC_FEATURE - Xaa51 IS Phe OR Tyr
  VARIANT 52: note = MISC_FEATURE - Xaa52 IS Arg, Lys, OR Val
  VARIANT 53: note = MISC_FEATURE - Xaa53 IS Met, Ile, Thr, Lys, OR Val
  VARIANT 54: note = MISC_FEATURE - Xaa54 IS Gly, Glu, Ala, Ser, Thr, OR Ile
  VARIANT 55: note = MISC_FEATURE - Xaa55 IS Gly, Ser, Asp, Arg, Lys, Gln, OR Asp
  VARIANT 56: note = MISC_FEATURE - Xaa56 IS Asn, Gly, Asp, Ala, OR Met
  VARIANT 57: note = MISC_FEATURE - Xaa57 IS ANY AMINO ACID OR IS ABSENT
  VARIANT 58: note = MISC_FEATURE - Xaa58 IS ANY AMINO ACID OR IS ABSENT
  VARIANT 59: note = MISC_FEATURE - Xaa59 IS ANY AMINO ACID OR IS ABSENT
  VARIANT 60: note = MISC_FEATURE - Xaa60 IS ANY AMINO ACID OR IS ABSENT
  VARIANT 61: note = MISC_FEATURE - Xaa61 IS ANY AMINO ACID OR IS ABSENT
  VARIANT 62: note = MISC_FEATURE - Xaa62 IS ANY AMINO ACID OR IS ABSENT
  VARIANT 64: note = MISC_FEATURE - Xaa64 IS Ala, Gly, Glu, OR Thr, OR IS ABSENT
  VARIANT 65: note = MISC_FEATURE - Xaa65 IS Ile, Arg, His, Tyr, OR IS ABSENT
  VARIANT 66: note = MISC_FEATURE - Xaa66 IS Pro OR Leu
  VARIANT 67: note = MISC_FEATURE - Xaa67 IS Ile, Leu, OR Met
  VARIANT 68: note = MISC_FEATURE - Xaa68 IS Thr, Tyr, Leu, OR Phe
  VARIANT 69: note = MISC_FEATURE - Xaa69 IS Val, Tyr, Phe, Leu, OR Arg
  VARIANT 70: note = MISC_FEATURE - Xaa70 IS Met, Ile, Arg, OR Lys
  VARIANT 71: note = MISC_FEATURE - Xaa71 IS Glu, Asp, OR Gln
  VARIANT 72: note = MISC_FEATURE - Xaa72 IS Tyr, Phe, OR Met
  VARIANT 73: note = MISC_FEATURE - Xaa73 IS Thr, Ala, Tyr, Arg, OR Phe
  VARIANT 74: note = MISC_FEATURE - Xaa74 IS Glu, Asp, Asn, OR Leu
  VARIANT 76: note = MISC_FEATURE - Xaa76 IS Ser, Glu, Asp, Gln, OR Gly
  VARIANT 77: note = MISC_FEATURE - Xaa77 IS Tyr, Pro, Thr, OR Asp
  VARIANT 78: note = MISC_FEATURE - Xaa78 IS Asn, Arg, Asp, Lys, Gln, OR Val
  VARIANT 79: note = MISC_FEATURE - Xaa79 IS Lys, Gln, Glu, OR Arg
  VARIANT 80: note = MISC_FEATURE - Xaa80 IS Ser, His, Val, Pro, Glu, OR Leu
  VARIANT 81: note = MISC_FEATURE - Xaa81 IS Leu OR Phe
  VARIANT 82: note = MISC_FEATURE - Xaa82 IS Gly OR Ser
  VARIANT 83: note = MISC_FEATURE - Xaa83 IS Ala, Tyr, Arg, Thr, OR Glu
  VARIANT 85: note = MISC_FEATURE - Xaa85 IS Pro, Arg, Lys, Ser, Ala, OR Leu
  VARIANT 86: note = MISC_FEATURE - Xaa86 IS Ile, Tyr, Arg, Val, Leu, Met, OR Lys
  VARIANT 87: note = MISC_FEATURE - Xaa87 IS Arg, Lys, Ser, OR Gln
  VARIANT 88: note = MISC_FEATURE - Xaa88 IS Thr, Asn, OR Ser
  VARIANT 89: note = MISC_FEATURE - Xaa89 IS Gln, Pro, Thr, Leu, OR Ala
  VARIANT 90: note = MISC_FEATURE - Xaa90 IS Pro, Gly, Ser, Asp, OR Gln
  VARIANT 91: note = MISC_FEATURE - Xaa91 IS Arg, Phe, Met, Trp, OR Tyr
  VARIANT 93: note = MISC_FEATURE - Xaa93 IS Asn, Asp, Trp, Ser, Leu, Lys, OR Ala
  VARIANT 94: note = MISC_FEATURE - Xaa94 IS Ser, Thr, Arg, Glu, Pro, OR Val, OR IS ABSENT
  VARIANT 95: note = MISC_FEATURE - XAA95 is Tyr, Phe, Pro, Arg, Ser, OR Asp
  VARIANT 96: note = MISC_FEATURE - Xaa96 IS Tyr, Leu, OR Ser
  VARIANT 97: note = MISC_FEATURE - Xaa97 IS Asp, Ala, Val, OR Thr
  VARIANT 98: note = MISC_FEATURE - Xaa98 IS Ser, Gly, Met, Lys, OR Pro
  VARIANT 99: note = MISC_FEATURE - Xaa99 IS Phe, Tyr, Thr, OR Ser
  VARIANT 100: note = MISC_FEATURE - Xaa100 IS Ser, Ala, Met, OR Thr
  VARIANT 101: note = MISC_FEATURE - Xaa101 IS Ala, Tyr, Phe, OR Leu
  VARIANT 102: note = MISC_FEATURE - Xaa102 IS Val, Pro, Thr, Ile, OR Leu
  VARIANT 103: note = MISC_FEATURE - Xaa103 IS Ser, Thr, OR Gly
  VARIANT 104: note = MISC_FEATURE - Xaa104 IS Glu, Asp, OR Arg
  VARIANT 105: note = MISC_FEATURE - Xaa105 IS Asp OR Asn
  VARIANT 106: note = MISC_FEATURE - Xaa106 IS Asn, Glu, OR Gly
  VARIANT 107: note = MISC_FEATURE - Xaa107 IS Leu OR Ala
  VARIANT 109: note = MISC_FEATURE - Xaa109 IS Phe OR Leu
  VARIANT 110: note = MISC_FEATURE - Xaa110 IS Leu, Ile, Val, OR Thr
  VARIANT 111: note = MISC_FEATURE - Xaa111 IS Met, Ile, Phe, OR Leu
  VARIANT 112: note = MISC_FEATURE - Xaa112 IS His, Ala, Val, Met, OR Phe
  VARIANT 113: note = MISC_FEATURE - Xaa113 IS Ala OR Ser
  VARIANT 115: note = MISC_FEATURE - Xaa115 IS Phe, Arg, Gln, Pro, OR Ala
  VARIANT 116: note = MISC_FEATURE - Xaa116 IS Glu, Leu, Phe, OR Ala
  VARIANT 117: note = MISC_FEATURE - Xaa117 IS Thr, Val, Asn, OR Leu
  VARIANT 118: note = MISC_FEATURE - Xaa118 IS Ala, Glu, Ser, Asp, OR Gln
  VARIANT 120: note = MISC_FEATURE - Xaa120 IS Thr, Gln, OR Leu
  VARIANT 122: note = MISC_FEATURE - Xaa122 IS Leu, Arg, OR Thr
  VARIANT 123: note = MISC_FEATURE - Xaa123 IS Arg OR Leu
  VARIANT 124: note = MISC_FEATURE - Xaa124 IS Leu, Ala, Val, OR Thr
  VARIANT 125: note = MISC_FEATURE - Xaa125 IS Val, Leu, OR Ile
  VARIANT 126: note = MISC_FEATURE - Xaa126 IS Lys, Tyr, Ser, Ile, Val, Thr, OR Gln
  VARIANT 127: note = MISC_FEATURE - Xaa127 IS Ile OR Val
  VARIANT 128: note = MISC_FEATURE - Xaa128 IS Asn, Asp, Glu, OR Gly
  VARIANT 129: note = MISC_FEATURE - Xaa129 IS Asp, Gly, Asn, OR Arg
  VARIANT 130: note = MISC_FEATURE - Xaa130 IS Trp, Thr, Val, Glu, Asp, Ser, OR Phe
  VARIANT 131: note = MISC_FEATURE - Xaa131 IS Thr, Val, Asn, Pro, Phe, Met, OR Ala
  VARIANT 132: note = MISC_FEATURE - Xaa132 IS Glu, Ala, Ile, Thr, Phe, OR Gln
  VARIANT 133: note = MISC_FEATURE - Xaa133 IS Ile, Tyr, Leu, Val, Ser, OR Thr
  VARIANT 134: note = MISC_FEATURE - Xaa134 IS Thr, Ser, OR Ala
  VARIANT 135: note = MISC_FEATURE - Xaa135 IS Gln, Asp, OR Leu
  VARIANT 136: note = MISC_FEATURE - Xaa136 IS Phe, Ile, OR Val
  VARIANT 137: note = MISC_FEATURE - Xaa137 IS Ile, Met, Leu, OR Thr
  VARIANT 138: note = MISC_FEATURE - Xaa138 IS Leu, Val, OR Phe

SEQ ID NO: 746 - protein, 138 aa, synthetic construct
  REGION 1..138: note = Synthetic sequence derived from human thought.
  VARIANT 2: note = MISC_FEATURE - X2aa IS Tyr, Arg, OR Lys
  VARIANT 3: note = MISC_FEATURE - Xaa3 IS Tyr OR His
  VARIANT 4: note = MISC_FEATURE - Xaa4 IS Ala, Thr, Val, OR Leu
  VARIANT 5: note = MISC_FEATURE - Xaa5 IS Val, Thr, Arg, OR Ile
  VARIANT 6: note = MISC_FEATURE - Xaa6 IS Leu, Ser, Pro, Tyr, OR Arg
  VARIANT 7: note = MISC_FEATURE - Xaa7 IS Glu, Ala, Leu, Thr, Asp, Met, OR Val
  VARIANT 8: note = MISC_FEATURE - Xaa8 IS ANY AMINO ACID OR IS ABSENT
  VARIANT 9: note = MISC_FEATURE - Xaa9 IS Arg, Ala, Asp, OR Glu
  VARIANT 10: note = MISC_FEATURE - Xaa10 IS Ala, Pro, Asn, Ser, OR Gly
  VARIANT 12: note = MISC_FEATURE - Xaa12 IS Arg, Asp, Gly, OR Ser
  VARIANT 13: note = MISC_FEATURE - Xaa13 IS Ser, Met, Val, OR Phe
  VARIANT 14: note = MISC_FEATURE - Xaa14 IS Val, Leu, OR Ala
  VARIANT 15: note = MISC_FEATURE - Xaa15 IS Leu, Ala, Val, OR Glu
  VARIANT 17: note = MISC_FEATURE - Xaa17 IS Asn, Ile, OR Trp
  VARIANT 18: note = MISC_FEATURE - Xaa18 IS Ala, Ser, OR Pro
  VARIANT 19: note = MISC_FEATURE - Xaa19 IS Pro, Asp, Phe, Ile, Glu, OR Asn
  VARIANT 20: note = MISC_FEATURE - Xaa20 IS Ser, Pro, Thr, OR Gly
  VARIANT 21: note = MISC_FEATURE - Xaa21 IS Glu, Gln, Asp, OR Asn
  VARIANT 22: note = MISC_FEATURE - Xaa22 IS Ala, Val, Pro, OR Ile
  VARIANT 23: note = MISC_FEATURE - Xaa23 IS Pro, Gly, Asp, Val, Lys, OR Glu
  VARIANT 24: note = MISC_FEATURE - Xaa24 IS Gln, Arg, Tyr, Ala, Ser, OR Asp
  VARIANT 25: note = MISC_FEATURE - Xaa25 IS Ile, Thr, Leu, Val, OR Met
  VARIANT 26: note = MISC_FEATURE - Xaa26 IS Val, Leu, OR Ile
  VARIANT 27: note = MISC_FEATURE - Xaa27 IS Arg, Trp, Asn, Leu, OR Ser
  VARIANT 28: note = MISC_FEATURE - Xaa28 IS Arg, Glu, Ser, Thr, OR Ala
  VARIANT 29: note = MISC_FEATURE - Xaa29 IS Ala, Leu, Gln, OR Ile
  VARIANT 30: note = MISC_FEATURE - Xaa30 IS ANY AMINO ACID OR IS ABSENT
  VARIANT 31: note = MISC_FEATURE - Xaa31 IS ANY AMINO ACID OR IS ABSENT
  VARIANT 32: note = MISC_FEATURE - Xaa32 IS ANY AMINO ACID OR IS ABSENT
  VARIANT 33: note = MISC_FEATURE - Xaa33 IS ANY AMINO ACID OR IS ABSENT
  VARIANT 34: note = MISC_FEATURE - Xaa34 IS ANY AMINO ACID OR IS ABSENT
  VARIANT 35: note = MISC_FEATURE - Xaa35 IS ANY AMINO ACID OR IS ABSENT
  VARIANT 36: note = MISC_FEATURE - Xaa36 is Pro, Val, Ser, Leu, OR Gln
  VARIANT 37: note = MISC_FEATURE - Xaa37 IS ANY AMINO ACID OR IS ABSENT
  VARIANT 38: note = MISC_FEATURE - Xaa38 IS ANY AMINO ACID OR IS ABSENT
  VARIANT 39: note = MISC_FEATURE - Xaa39 IS ANY AMINO ACID OR IS ABSENT
  VARIANT 40: note = MISC_FEATURE - Xaa40 IS ANY AMINO ACID OR IS ABSENT
  VARIANT 41: note = MISC_FEATURE - Xaa41 IS ANY AMINO ACID OR IS ABSENT
  VARIANT 42: note = MISC_FEATURE - Xaa42 IS ANY AMINO ACID OR IS ABSENT
  VARIANT 43: note = MISC_FEATURE - Xaa43 IS ANY AMINO ACID OR IS ABSENT
  VARIANT 45: note = MISC_FEATURE - Xaa45 IS Asn, Arg, Asp, OR Glu
  VARIANT 46: note = MISC_FEATURE - Xaa46 IS Leu OR Ala
  VARIANT 47: note = MISC_FEATURE - Xaa47 IS Thr, His, Leu, OR Ser
  VARIANT 48: note = MISC_FEATURE - Xaa48 IS Ile, Val, OR Leu
  VARIANT 49: note = MISC_FEATURE - Xaa49 IS Ala, Ile, Ser, OR Val
  VARIANT 51: note = MISC_FEATURE - Xaa51 IS Phe OR Tyr
  VARIANT 52: note = MISC_FEATURE - Xaa52 IS Arg, Lys, OR Val
  VARIANT 53: note = MISC_FEATURE - Xaa53 IS Met, Ile, Thr, Leu, OR Val
  VARIANT 54: note = MISC_FEATURE - Xaa54 IS Gly, Glu, Ala, Ser, Thr, OR Ile
  VARIANT 55: note = MISC_FEATURE - Xaa55 IS Gly, Ser, Asp, Arg, Lys, Gln, OR Asp
  VARIANT 56: note = MISC_FEATURE - Xaa56 IS Asn, Arg, Asp, Ala, OR Met
  VARIANT 57: note = MISC_FEATURE - Xaa57 IS ANY AMINO ACID OR IS ABSENT
  VARIANT 58: note = MISC_FEATURE - Xaa58 IS ANY AMINO ACID OR IS ABSENT
  VARIANT 59: note = MISC_FEATURE - Xaa59 IS ANY AMINO ACID OR IS ABSENT
  VARIANT 60: note = MISC_FEATURE - Xaa60 IS ANY AMINO ACID OR IS ABSENT
  VARIANT 61: note = MISC_FEATURE - Xaa61 IS ANY AMINO ACID OR IS ABSENT
  VARIANT 62: note = MISC_FEATURE - Xaa62 IS ANY AMINO ACID OR IS ABSENT
  VARIANT 64: note = MISC_FEATURE - Xaa64 IS Ala, Gly, Glu, OR Thr, OR IS ABSENT
  VARIANT 65: note = MISC_FEATURE - Xaa65 IS Ile, Arg, His, Tyr, OR IS ABSENT
  VARIANT 66: note = MISC_FEATURE - X66 IS Pro OR Leu
  VARIANT 67: note = MISC_FEATURE - Xaa67 IS Ile, Leu, OR Met
  VARIANT 68: note = MISC_FEATURE - Xaa68 IS Thr, Tyr, Leu, OR Phe
  VARIANT 69: note = MISC_FEATURE - Xaa69 IS Val, Tyr, Phe, Leu, OR Arg
  VARIANT 70: note = MISC_FEATURE - Xaa70 IS Met, Ile, Arg, OR Lys
  VARIANT 73: note = MISC_FEATURE - Xaa73 IS Thr, Ala, Tyr, Arg, OR Phe
  VARIANT 74: note = MISC_FEATURE - Xaa74 IS Glu, Asp, Asn, OR Leu
  VARIANT 76: note = MISC_FEATURE - Xaa76 IS Ser, Glu, Asp, Gln, OR Gly
  VARIANT 77: note = MISC_FEATURE - Xaa77 IS Tyr, Pro, Thr, OR Asp
  VARIANT 78: note = MISC_FEATURE - Xaa78 IS Asn, Arg, Asp, Lys, Gln, OR Val
  VARIANT 79: note = MISC_FEATURE - Xaa79 IS Lys, Gln, Glu, OR Arg
  VARIANT 80: note = MISC_FEATURE - Xaa80 IS Ser, His, Val, Pro, Glu, OR Leu
  VARIANT 81: note = MISC_FEATURE - Xaa81 IS Leu OR Phe
  VARIANT 83: note = MISC_FEATURE - Xaa83 IS Ala, Tyr, Arg, Thr, OR Glu
  VARIANT 85: note = MISC_FEATURE - Xaa85 IS Pro, Arg, Lys, Ser, Ala, OR Leu
  VARIANT 86: note = MISC_FEATURE - Xaa86 IS Ile, Tyr, Arg, Val, Leu, Met, OR Lys
  VARIANT 87: note = MISC_FEATURE - Xaa87 IS Arg, Lys, Ser, OR Gln
  VARIANT 88: note = MISC_FEATURE - Xaa88 IS Thr, Asn, OR Ser
  VARIANT 89: note = MISC_FEATURE - Xaa89 IS Gln, Pro, Thr, Leu, OR Ala
  VARIANT 90: note = MISC_FEATURE - Xaa90 IS Pro, Gly, Ser, Asp, OR Gln
  VARIANT 91: note = MISC_FEATURE - Xaa91 IS Arg, Phe, Met, Trp, OR Tyr
  VARIANT 93: note = MISC_FEATURE - Xaa93 IS Asn, Asp, Trp, Ser, Leu, Lys, OR Ala
  VARIANT 94: note = MISC_FEATURE - Xaa94 IS Ser, Thr, Arg, Glu, Pro, OR Val, OR IS ABSENT
  VARIANT 95: note = MISC_FEATURE - Xaa95 IS Tyr, Phe, Pro, Arg, Ser, OR Asp
  VARIANT 96: note = MISC_FEATURE - Xaa96 IS Tyr, Leu, OR Ser
  VARIANT 97: note = MISC_FEATURE - Xaa97 IS Asp, Ala, Val, OR Thr
  VARIANT 98: note = MISC_FEATURE - Xaa98 IS Ser, Gly, Met, Lys, OR Pro
  VARIANT 99: note = MISC_FEATURE - Xaa99 IS Phe, Tyr, Thr, OR Ser
  VARIANT 100: note = MISC_FEATURE - Xaa100 IS Ser, Ala, Met, OR Thr
  VARIANT 101: note = MISC_FEATURE - Xaa101 IS Ala, Tyr, Phe, OR Leu
  VARIANT 102: note = MISC_FEATURE - Xaa102 IS Val, Pro, Thr, Ile, OR Leu
  VARIANT 103: note = MISC_FEATURE - Xaa103 IS Ser, Thr, OR Gly
  VARIANT 104: note = MISC_FEATURE - Xaa104 IS Glu, Asp, OR Arg
  VARIANT 106: note = MISC_FEATURE - Xaa106 IS Asn, Glu, OR Gly
  VARIANT 109: note = MISC_FEATURE - Xaa109 IS Phe OR Leu
  VARIANT 110: note = MISC_FEATURE - Xaa110 IS Leu, Ile, Val, OR Thr
  VARIANT 111: note = MISC_FEATURE - Xaa111 IS Met, Ile, Phe, OR Leu
  VARIANT 112: note = MISC_FEATURE - Xaa112 IS His, Ala, Val, Met, OR Phe
  VARIANT 113: note = MISC_FEATURE - Xaa113 IS Ala OR Ser
  VARIANT 115: note = MISC_FEATURE - Xaa115 IS Phe, Arg, Gln, Pro, OR Ala
  VARIANT 116: note = MISC_FEATURE - Xaa116 IS Glu, Leu, Phe, OR Ala
  VARIANT 117: note = MISC_FEATURE - Xaa117 IS Thr, Val, Asn, OR Leu
  VARIANT 118: note = MISC_FEATURE - Xaa118 IS Ala, Glu, Ser, Asp, OR Gln
  VARIANT 120: note = MISC_FEATURE - Xaa120 IS Thr, Gln, OR Leu
  VARIANT 122: note = MISC_FEATURE - Xaa122 IS Leu, Arg, OR Thr
  VARIANT 124: note = MISC_FEATURE - Xaa124 IS Leu, Ala, Val, OR Thr
  VARIANT 125: note = MISC_FEATURE - Xaa125 IS Val, Leu, OR Ile
  VARIANT 126: note = MISC_FEATURE - Xaa126 IS Lys, Tyr, Ser, Ile, Val, Thr, OR Gln
  VARIANT 127: note = MISC_FEATURE - Xaa127 IS Ile OR Val
  VARIANT 128: note = MISC_FEATURE - Xaa128 IS Asn, Asp, Glu, OR Gly
  VARIANT 129: note = MISC_FEATURE - Xaa129 IS Asp, Gly, Asn, OR Arg
  VARIANT 130: note = MISC_FEATURE - Xaa130 IS Trp, Thr, Val, Glu, Asp, Ser, OR Phe
  VARIANT 131: note = MISC_FEATURE - Xaa131 IS Thr, Val, Asn, Pro, Phe, Met, OR Ala
  VARIANT 132: note = MISC_FEATURE - Xaa132 IS Glu, Ala, Ile, Thr, Phe, OR Gln
  VARIANT 133: note = MISC_FEATURE - Xaa133 IS Ile, Tyr, Leu, Val, Ser, OR Thr
  VARIANT 134: note = MISC_FEATURE - Xaa134 IS Thr, Ser, OR Ala
  VARIANT 135: note = MISC_FEATURE - Xaa135 IS Gln, Asp, OR Leu
  VARIANT 136: note = MISC_FEATURE - Xaa136 IS Phe, Ile, OR Val
  VARIANT 137: note = MISC_FEATURE - Xaa137 IS Ile, Met, Leu, OR Thr
  VARIANT 138: note = MISC_FEATURE - Xaa138 IS Leu, Val, OR Phe
VXXXXXXXXXCXXXXLXXXXXXXXXXXXXXXXXXXXXXXXXXXYXXXXXWXXXXXXXXXXXXCXXXXXXXEYXXCXXXXXXGXCXXXXXXXWXXXXXXXXXXXXDXLGXXXXXPXXXXGXYXRXXXXXXXXXXXXXXX

SEQ ID NO: 747 - skipped

SEQ ID NO: 748 - protein, 28 aa, synthetic construct
  REGION 1..28: note = Synthetic construct derived from human thought.
  VARIANT 2: note = MISC_FEATURE - Xaa2 IS ANY AMINO ACID
  VARIANT 4: note = MISC_FEATURE - Xaa4 IS ANY AMINO ACID
  VARIANT 5: note = MISC_FEATURE - Xaa5 IS ANY AMINO ACID
  VARIANT 6: note = MISC_FEATURE - Xaa6 IS ANY AMINO ACID
  VARIANT 8: note = MISC_FEATURE - Xaa8 IS ANY AMINO ACID
  VARIANT 10: note = MISC_FEATURE - Xaa10 IS ANY AMINO ACID OR IS ABSENT
  VARIANT 12: note = MISC_FEATURE - Xaa12 IS ANY AMINO ACID OR IS ABSENT
  VARIANT 13: note = MISC_FEATURE - Xaa13 IS ANY AMINO ACID
  VARIANT 15: note = MISC_FEATURE - Xaa15 IS ANY AMINO ACID OR IS ABSENT
  VARIANT 16: note = MISC_FEATURE - Xaa16 IS ANY AMINO ACID OR IS ABSENT
  VARIANT 17: note = MISC_FEATURE - Xaa17 IS ANY AMINO ACID OR IS ABSENT
  VARIANT 18: note = MISC_FEATURE - Xaa18 IS ANY AMINO ACID OR IS ABSENT
  VARIANT 19: note = MISC_FEATURE - Xaa19 IS ANY AMINO ACID OR IS ABSENT
  VARIANT 20: note = MISC_FEATURE - Xaa20 IS ANY AMINO ACID
  VARIANT 21: note = MISC_FEATURE - Xaa21 IS ANY AMINO ACID
  VARIANT 22: note = MISC_FEATURE - Xaa22 IS ANY AMINO ACID
  VARIANT 23: note = MISC_FEATURE - Xaa23 IS ANY AMINO ACID
  VARIANT 25: note = MISC_FEATURE - Xaa25 IS ANY AMINO ACID
  VARIANT 26: note = MISC_FEATURE - Xaa26 IS ANY AMINO ACID OR IS ABSENT
  VARIANT 27: note = MISC_FEATURE - Xaa27 IS ANY AMINO ACID OR IS ABSENT
VXVXXXGXLXPXXFXXXXXXXXXVXXXY

SEQ ID NO: 749 - protein, 27 aa, synthetic construct
  REGION 1..27: note = Synthetic construct derived from human thought.
  VARIANT 10: note = MISC_FEATURE - Xaa10 IS ANY AMINO ACID OR IS ABSENT
  VARIANT 14: note = MISC_FEATURE - Xaa14 IS ANY AMINO ACID OR IS ABSENT
  VARIANT 15: note = MISC_FEATURE - Xaa15 IS ANY AMINO ACID OR IS ABSENT
  VARIANT 16: note = MISC_FEATURE - Xaa16 IS ANY AMINO ACID OR IS ABSENT
  VARIANT 17: note = MISC_FEATURE - Xaa17 IS ANY AMINO ACID OR IS ABSENT
  VARIANT 18: note = MISC_FEATURE - Xaa18 IS ANY AMINO ACID
  VARIANT 19: note = MISC_FEATURE - Xaa19 IS ANY AMINO ACID
  VARIANT 20: note = MISC_FEATURE - Xaa20 IS ANY AMINO ACID
  VARIANT 21: note = MISC_FEATURE - Xaa21 IS ANY AMINO ACID
  VARIANT 24: note = MISC_FEATURE - Xaa24 IS ANY AMINO ACID
  VARIANT 25: note = MISC_FEATURE - Xaa25 IS ANY AMINO ACID OR IS ABSENT
  VARIANT 26: note = MISC_FEATURE - Xaa26 IS ANY AMINO ACID OR IS ABSENT
VTVDSIGMLXPRFXXXXXXXXTVXXXY

SEQ ID NO: 750 - protein, 35 aa, synthetic construct
  REGION 1..35: note = Synthetic construct derived from human thought
  VARIANT 2: note = MISC_FEATURE - Xaa2 IS ANY AMINO ACID OR IS ABSENT
  VARIANT 3: note = MISC_FEATURE - Xaa3 IS ANY AMINO ACID
  VARIANT 4: note = MISC_FEATURE - Xaa4 IS ANY AMINO ACID
  VARIANT 5: note = MISC_FEATURE - Xaa5 IS ANY AMINO ACID
  VARIANT 6: note = MISC_FEATURE - Xaa6 IS ANY AMINO ACID
  VARIANT 7: note = MISC_FEATURE - Xaa7 IS ANY AMINO ACID
  VARIANT 18: note = MISC_FEATURE - Xaa18 IS ANY AMINO ACID OR IS ABSENT
  VARIANT 22: note = MISC_FEATURE - Xaa22 IS ANY AMINO ACID OR IS ABSENT
  VARIANT 23: note = MISC_FEATURE - Xaa23 IS ANY AMINO ACID OR IS ABSENT
  VARIANT 24: note = MISC_FEATURE - Xaa24 IS ANY AMINO ACID OR IS ABSENT
  VARIANT 25: note = MISC_FEATURE - Xaa25 IS ANY AMINO ACID OR IS ABSENT
  VARIANT 26: note = MISC_FEATURE - Xaa26 IS ANY AMINO ACID
  VARIANT 27: note = MISC_FEATURE - Xaa27 IS ANY AMINO ACID
  VARIANT 28: note = MISC_FEATURE - Xaa28 IS ANY AMINO ACID
  VARIANT 29: note = MISC_FEATURE - Xaa29 IS ANY AMINO ACID
  VARIANT 32: note = MISC_FEATURE - Xaa32 IS ANY AMINO ACID OR IS ABSENT
  VARIANT 33: note = MISC_FEATURE - Xaa33 IS ANY AMINO ACID OR IS ABSENT
  VARIANT 34: note = MISC_FEATURE - Xaa34 IS ANY AMINO ACID OR IS ABSENT
SXXXXXXGVTVDSIGMLXPRFXXXXXXXXTVXXXY

SEQ ID NO: 751 - protein, 38 aa, synthetic construct
  REGION 1..38: note = Synthetic construct derived from human thought.
  VARIANT 1: note = MISC_FEATURE - Xaa1 IS ANY AMINO ACID OR IS ABSENT
  VARIANT 2: note = MISC_FEATURE - Xaa2 IS ANY AMINO ACID OR IS ABSENT
  VARIANT 3: note = MISC_FEATURE - Xaa3 IS ANY AMINO ACID OR IS ABSENT
  VARIANT 4: note = MISC_FEATURE - Xaa4 IS ANY AMINO ACID OR IS ABSENT
  VARIANT 5: note = MISC_FEATURE - Xaa5 IS ANY AMINO ACID
  VARIANT 6: note = MISC_FEATURE - Xaa6 IS ANY AMINO ACID
  VARIANT 7: note = MISC_FEATURE - Xaa7 IS ANY AMINO ACID
  VARIANT 8: note = MISC_FEATURE - Xaa8 IS ANY AMINO ACID
  VARIANT 9: note = MISC_FEATURE - Xaa9 IS ANY AMINO ACID
  VARIANT 10: note = MISC_FEATURE - Xaa10 IS ANY AMINO ACID
  VARIANT 23: note = MISC_FEATURE - Xaa23 IS ANY AMINO ACID OR IS ABSENT
  VARIANT 24: note = MISC_FEATURE - Xaa24 IS ANY AMINO ACID OR IS ABSENT
  VARIANT 25: note = MISC_FEATURE - Xaa25 IS ANY AMINO ACID OR IS ABSENT
  VARIANT 26: note = MISC_FEATURE - Xaa26 IS ANY AMINO ACID OR IS ABSENT
  VARIANT 27: note = MISC_FEATURE - Xaa27 IS ANY AMINO ACID
  VARIANT 28: note = MISC_FEATURE - Xaa28 IS ANY AMINO ACID
  VARIANT 29: note = MISC_FEATURE - Xaa29 IS ANY AMINO ACID
  VARIANT 30: note = MISC_FEATURE - Xaa30 IS ANY AMINO ACID
  VARIANT 31: note = MISC_FEATURE - Xaa31 IS ANY AMINO ACID
  VARIANT 34: note = MISC_FEATURE - Xaa34 IS ANY AMINO ACID OR IS ABSENT
  VARIANT 35: note = MISC_FEATURE - Xaa35 IS ANY AMINO ACID OR IS ABSENT
  VARIANT 36: note = MISC_FEATURE - Xaa36 IS ANY AMINO ACID OR IS ABSENT
  VARIANT 37: note = MISC_FEATURE - Xaa37 IS ANY AMINO ACID OR IS ABSENT
XXXXXXXXXXVTVDSIGMLPRFXXXXXXXXXTVXXXXY

SEQ ID NO: 752 - skipped

SEQ ID NO: 753 - protein, 19 aa, synthetic construct
  REGION 1..19: note = Synthetic construct derived from human thought.
  VARIANT 2: note = MISC_FEATURE - Xaa2 IS ANY AMINO ACID
  VARIANT 4: note = MISC_FEATURE - Xaa4 IS ANY AMINO ACID
  VARIANT 6: note = MISC_FEATURE - Xaa6 IS ANY AMINO ACID
  VARIANT 8: note = MISC_FEATURE - Xaa8 IS ANY AMINO ACID OR IS ABSENT
  VARIANT 9: note = MISC_FEATURE - Xaa9 IS ANY AMINO ACID OR IS ABSENT
  VARIANT 10: note = MISC_FEATURE - Xaa10 IS ANY AMINO ACID
  VARIANT 11: note = MISC_FEATURE - Xaa11 IS ANY AMINO ACID
  VARIANT 12: note = MISC_FEATURE - Xaa12 IS ANY AMINO ACID
  VARIANT 13: note = MISC_FEATURE - Xaa13 IS ANY AMINO ACID
  VARIANT 14: note = MISC_FEATURE - Xaa14 IS ANY AMINO ACID
  VARIANT 15: note = MISC_FEATURE - Xaa15 IS ANY AMINO ACID
  VARIANT 16: note = MISC_FEATURE - Xaa16 IS ANY AMINO ACID
PXLXGXLXXXXXXXXXVYH

SEQ ID NO: 754 - protein, 37 aa, synthetic construct
  REGION 1..37: note = Synthetic construct derived from human thought.
  VARIANT 1: note = MISC_FEATURE - Xaa1 IS ANY AMINO ACID
  VARIANT 2: note = MISC_FEATURE - Xaa2 IS ANY AMINO ACID
  VARIANT 4: note = MISC_FEATURE - Xaa4 IS ANY AMINO ACID
  VARIANT 5: note = MISC_FEATURE - Xaa5 IS ANY AMINO ACID
  VARIANT 7: note = MISC_FEATURE - Xaa7 IS ANY AMINO ACID OR IS ABSENT
  VARIANT 8: note = MISC_FEATURE - Xaa8 IS ANY AMINO ACID OR IS ABSENT
  VARIANT 9: note = MISC_FEATURE - Xaa9 IS ANY AMINO ACID
  VARIANT 10: note = MISC_FEATURE - Xaa10 IS ANY AMINO ACID
  VARIANT 11: note = MISC_FEATURE - Xaa11 IS ANY AMINO ACID
  VARIANT 12: note = MISC_FEATURE - Xaa12 IS ANY AMINO ACID
  VARIANT 13: note = MISC_FEATURE - Xaa13 IS ANY AMINO ACID
  VARIANT 14: note = MISC_FEATURE - Xaa14 IS ANY AMINO ACID
  VARIANT 15: note = MISC_FEATURE - Xaa15 IS ANY AMINO ACID
  VARIANT 17: note = MISC_FEATURE - Xaa17 IS ANY AMINO ACID OR IS ABSENT
  VARIANT 18: note = MISC_FEATURE - Xaa18 IS ANY AMINO ACID OR IS ABSENT
  VARIANT 19: note = MISC_FEATURE - Xaa19 IS ANY AMINO ACID OR IS ABSENT
  VARIANT 20: note = MISC_FEATURE - Xaa20 IS ANY AMINO ACID OR IS ABSENT
  VARIANT 21: note = MISC_FEATURE - Xaa21 IS ANY AMINO ACID OR IS ABSENT
  VARIANT 22: note = MISC_FEATURE - Xaa22 IS ANY AMINO ACID OR IS ABSENT
  VARIANT 23: note = MISC_FEATURE - Xaa23 IS ANY AMINO ACID
  VARIANT 24: note = MISC_FEATURE - Xaa24 IS ANY AMINO ACID
  VARIANT 25: note = MISC_FEATURE - Xaa25 IS ANY AMINO ACID
  VARIANT 26: note = MISC_FEATURE - Xaa26 IS ANY AMINO ACID
  VARIANT 28: note = MISC_FEATURE - Xaa28 IS ANY AMINO ACID OR IS ABSENT
  VARIANT 30: note = MISC_FEATURE - Xaa30 IS ANY AMINO ACID
  VARIANT 33: note = MISC_FEATURE - Xaa33 IS ANY AMINO ACID
  VARIANT 34: note = MISC_FEATURE - Xaa34 IS ANY AMINO ACID
  VARIANT 36: note = MISC_FEATURE - Xaa36 IS ANY AMINO ACID
XXLXXLXXXXXXXXXVXXXXXXXXXXPXFXPPXXPXT

SEQ ID NO: 755 - protein, 17 aa, synthetic construct
  REGION 1..17: note = Synthetic construct derived from human thought
  VARIANT 2: note = MISC_FEATURE - Xaa2 IS ANY AMINO ACID
  VARIANT 4: note = MISC_FEATURE - Xaa4 IS ANY AMINO ACID
  VARIANT 6: note = MISC_FEATURE - Xaa6 IS ANY AMINO ACID
  VARIANT 7: note = MISC_FEATURE - Xaa7 IS ANY AMINO ACID
  VARIANT 8: note = MISC_FEATURE - Xaa8 IS ANY AMINO ACID
  VARIANT 9: note = MISC_FEATURE - Xaa9 IS ANY AMINO ACID
  VARIANT 10: note = MISC_FEATURE - Xaa10 IS ANY AMINO ACID
  VARIANT 11: note = MISC_FEATURE - Xaa11 IS ANY AMINO ACID
  VARIANT 12: note = MISC_FEATURE - Xaa12 IS ANY AMINO ACID
  VARIANT 13: note = MISC_FEATURE - Xaa7 IS ANY AMINO ACID
PXLXRXXXXXXXXRVYH